(19) United States
(12) Patent Application Publication
(10) Pub. No.: US 2025/0144291 A1
(43) Pub. Date: May 8, 2025

(54) ARTIFICIAL INTELLIGENCE BASED IMPLANTABLE DRUG DELIVERY SYSTEM

(71) Applicant: Manta Implant LLC, McLean, VA (US)

(72) Inventors: Raju S. DAVE, Gaithersburg, MD (US); Kenta ALTEN, Los Altos Hills, CA (US); Sanjay DALVI, Thane (West) (IN); Neil DAVEY, Gaithersburg, MD (US); Sonya DAVEY, Gaithersburg, MD (US); Eric FRISCHE, Addison, TX (US); Aadi GANNAVARAM, Potomac, MD (US); Tri Le, San Diego, CA (US); Ray RADAR, Springfield, VA (US); Xing SU, Menlo Park, CA (US); Sudhish SWAIN, Gaithersburg, MD (US); Sujay SWAIN, Gaithersburg, MD (US); Palamadai VENKATRAMAN, Edison, NJ (US); Himanshu VERMA, McLean, VA (US); Sachi VERMA, McLean, VA (US)

(73) Assignee: Manta Implant LLC, McLean, VA (US)

(21) Appl. No.: 18/781,451

(22) Filed: Jul. 23, 2024

Related U.S. Application Data
(60) Provisional application No. 63/515,130, filed on Jul. 23, 2023.
(60) Provisional application No. 63/617,057, filed on Jan. 2, 2024.
(60) Provisional application No. 63/617,754, filed on Jan. 4, 2024.
(60) Provisional application No. 63/566,519, filed on Mar. 18, 2024.
(60) Provisional application No. 63/574,330, filed on Apr. 4, 2024.

(51) Int. Cl. A61M 5/142; A61M 5/145; A61M 5/168; G16H 40/63

(52) U.S. Cl. CPC A61M 5/14276; A61M 5/14526; A61M 5/16804; G16H 40/63; A61M 2005/14513; A61M 2205/0283; A61M 2205/0294; A61M 2205/18; A61M 2205/3317; A61M 2205/3331; A61M 2205/3523; A61M 2205/50; A61M 2205/8206; A61M 2205/8243; A61M 2230/20; A61M 2250/00

(57) ABSTRACT

Embodiments relate to device, system and methods for personalised drug dosing via a device, the device comprising a first chamber comprising a drug reservoir unit; a second chamber comprising an electronic unit comprising a control component; a third chamber comprising a drug delivery unit and a flow control component controlled via the electronic unit; wherein the device is fully implanted in a subject during its intended operational modes, and wherein the device releases a body temperature stable drug formulation into the subject at a flow rate such that a variation in the flow rate is within ±15% by volume.

### INCORPORATION BY REFERENCE

This application claims priority from the U.S. Provisional Application No. 63/515,130 filed on Jul. 23, 2023, U.S. Provisional Application No. 63/617,057 filed on Jan. 2, 2024, U.S. Provisional Application No. 63/617,754 filed on Jan. 4, 2024, U.S. Provisional Application No. 63/566,519 filed on Mar. 18, 2024, and U.S. Provisional Application No. 63/574,330, filed Apr. 4, 2024, the content of which are incorporated herein by reference in their entirety.

### FIELD OF INVENTION

This disclosure relates to devices, systems, and methods for adjustable rate drug delivery via an implantable device wherein the flow rate of elution (i.e., release) of the drug from the device can be maintained substantially constant, increased or decreased with time, or even stopped momentarily.

### BACKGROUND

“Medication nonadherence for patients with chronic diseases is extremely common, affecting as many as 40% to 50% of patients who are prescribed medications for management of chronic conditions such as diabetes or hypertension. This nonadherence to prescribed treatment is thought to cause at least 100,000 preventable deaths and $100 billion in preventable medical costs per year. Despite this, the medical profession largely ignores medication nonadherence or sees it as a patient problem and not a physician or health system problem.” [Source: Kleinsinger F. The Unmet Challenge of Medication Nonadherence. Perm J. 2018; 22:18-033. doi: 10.7812/TPP/18-033. PMID: 30005722; PMCID: PMC6045499.]

“According to the World Health Organization (WHO), approximately 125,000 people with treatable ailments die each year in the United States because they do not take their medication properly. The WHO also reports that up to 25% of hospital admissions result from noncompliance.” [Source: Kim J, Combs K, Downs J, Tillman F. Medication Adherence: The Elephant in the Room. US Pharm. 2018; 43(1)30-34.]

“Not taking medication as prescribed can account for up to 50% of treatment failures. Studies back up the prevalence of patient noncompliance. A 2016 study found that a third of people living with kidney transplants don't take their anti-rejection medications.” [Source: Patzer, R, et al. Medication understanding, non-adherence, and clinical outcomes among adult kidney transplant recipients. Clinical Transplantation. 2016; 30(10):1294-1305. doi:10.1111/ctr.12821]

“An estimated 50% of people with cardiovascular (heart) disease and its major risk factors have poor adherence to prescribed medications. This failure can lead to additional health complications.” [Kronish I, Ye S. Adherence to Cardiovascular Medications: Lessons Learned and Future Directions. Prog Cardiovasc Dis. 2013; 55(6):590-600. doi:10.1016/j.pcad.2013.02.001]

Though thousands of effective biopharmaceuticals exist for a wide array of medical/clinical indications, adherence to drug administration remains a severe problem in fully treating these diseases. Given the difficulties in taking medication every day, or numerous times throughout the day, and the complexities in properly delivering a drug into the body, a small implantable device would be a convenient solution for a large number of patient populations. Such an implantable device would safely allow for the controlled release of a substance into the body, and this drug delivery platform could be used universally for a variety of conditions.

An important limitation in many existing drug delivery implants is that their drug release profiles are not well controlled. Though it may be required in certain cases, most drug release profiles in an implantable device are undesirable as the drug concentration decreases as a function of time as the drug runs out. The present disclosure overcomes these limitations and furthermore provides many other added benefits such as targeted delivery of the drug to a target (e.g., tissue or organ) in the body.

“Poor adherence to medication and prescribed health of medical related regimens is a recognized medical problem in the U.S. and abroad. At least a third of all medication-related hospital admissions are caused by poor medication adherence, and these events alone are estimated to cost $100 billion annually in the USA.” [PMID 18183470, J Gen intern Med. 2008 February; 23(2):216-8. Medication Adherence After Myocardial Infarction: A Long Way Left To Go. Choudhry N K, Winkelmayer W C.]

“One major drawback with implantable infusion pumps according to the prior art is that they cannot easily be optimized with regard to flowrate of the delivered drug and the volume of drug stored in the pump.” [WO2000066201A1 published on 2000 Nov. 9]

“These proposed IVR acceptance criteria for daily drug release are significantly wider than other drug delivery devices such as pen injectors and infusion devices. For example, injection products, such as pen injectors, conforming to the International Organization for Standardization (ISO) 11608-1 standard (Needle-Based Injection System for Medical Use—Requirements and Test Methods—Part 1: Needle-based Injection Systems) (International Organization for Standardization 2022) generally have a dose accuracy of no wider than ±5% of the intended target dose. In addition, infusion products such as on-body insulin pumps or large volume infusion pumps commonly allow for a variation of 5 to 15% of the specified infusion rate . . . [T]he ITCA 650 safety analyses along with the inconsistent device performance and the variable PK may indicate that the inconsistency in delivered dose is contributing to an unexpectedly unfavorable safety profile for a drug that otherwise has not been associated with these safety findings . . . benefit-risk assessment for the ITCA 650 drug-device combination product is unfavorable.[FDA Briefing Information; Endocrinologic and Metabolic Drugs Advisory Committee dated Sep. 21, 2023]

Considering the knowledge of persons skilled in the art, there is a long-felt need to address the shortcomings in the prior art and provide a system that is capable of implementing comprehensive strategies addressing issues related to clinical efficacy, toxicity, drug properties, and personalized drug dosing. It would be advantageous to have a system, method and device that considers at least some of the issues discussed above, as well as possibly other issues.

### SUMMARY

The present disclosure describes one or more aspects of methods and systems enabling the steady delivery of drugs in their most stable formulation for long-term treatment thereby improving tolerability by addressing concerns typically associated with medications.

An embodiment relates to a tamper-proof implantable device having from 3 mm to 6 mm diameter tubular form (preferably, between 4 mm to 5 mm diameter), wherein the implantable device is inserted subcutaneously and non-surgically by an injector device in a human body via an incision of width 6 mm or less by a doctor or a nurse practitioner in a doctor's office or in an outpatient clinic; the implantable device comprising a casing comprising a pump, a piston, a drug chamber comprising a drug, and an opening for release of the drug from the implantable device into a body of a subject; wherein the implantable device is configured to be located in a subcutaneous region within the body of the subject during delivery of the drug into the body of the subject; wherein the implantable device is configured to deliver multiple doses of the drug within the body of the subject with a dose-to-dose variation of ±25% or less by volume (preferably ±20% or less, more preferably ±15% or less and most preferably ±10% or less); wherein the implantable device further comprises power supply and electronics; and wherein the implantable device is configured to notify the subject in case of failure of the implantable device.

An embodiment relates to a system comprising an AI-based implantable drug delivery system; wherein the AI-based implantable delivery system comprises; an implantable device (described above) that serves as a drug delivery engine, dispensing a drug subcutaneously in a controlled and on-demand manner within a body of a subject; a body temperature stable drug formulation that ensures that the drug does not degrade within the body of the subject at a body temperature of the subject for at least 6 months (preferably, 1 year, more preferably 2 years, and most preferably 3 years); an implantable biosensor that provides real-time monitoring of a drug level and/or a health-related biomarker; and AI Integration that integrates and analyzes data to optimize drug delivery in real-time; wherein the subject could be a human being and the body temperature of the subject is in a range from 95° F. to 105° F.

In an embodiment, the drug could be contained in a bead, wherein, preferably, the drug and/or the beads comprise a targeting material or targeting molecule that binds to a certain organ, tissue, object, or a specific site within the body of the subject.

In an embodiment, the bead comprises a core and a shell, wherein preferably, the shell comprises a stimuli-responsive polymer, more preferably a stimuli-responsive biodegradable polymer, configured to break the shell, before or after implanting or attaching the implantable device in or on a body of a subject, when the beads are exposed to an external stimulus.

Preferably, the shell comprises a first material and a second material; wherein the first material comprises a metal-containing material or a first biodegradable material; wherein the second material comprises a second biodegradable material; wherein the first material is distributed in the second material; wherein the first material is configured to create openings in the second material; wherein the openings allow the drug to flow from within the beads to outside the beads.

Preferably, the metal-containing material is configured to form openings in the shell, before or after implanting or attaching the implantable device in or on the body of a subject, when the beads are exposed to an external stimulus.

Preferably, the metal-containing material comprises metallic particles. Preferably, metallic particles comprise iron-containing or manganese-containing particles, or an iron-containing or manganese-containing polymer.

Preferably, the first and second materials comprise polymers. Preferably, the first material comprises polylactic acid (PLA) or an iron-containing polymer and the second biodegradable material comprises poly ε-caprolactone (PCL). Preferably, the core comprises a drug and a polymer. Preferably, the core comprises an emulsion of the drug and the polymer.

Preferably, the desired flow rate of elution of the drug is substantially constant. Preferably, the desired flow rate of elution of the drug is substantially constant for a first period of time and substantially zero for a second period of time, or vice versa, or any combinations thereof. Preferably, the desired flow rate of elution of the drug is increasing or decreasing with time, or any combinations thereof.

The implantable device could further comprise a sensor configured to monitor a health parameter. In an embodiment, the sensor is configured to measure the concentration of the drug in the body of the subject. The implantable device could further comprise a side wall at or near a second end of the casing, wherein the casing is substantially tubular having at least the first end of the casing and the second end of the casing, and the second end of the casing is opposite the first end of the casing. The implantable device could further comprise a sonicator or a plate with holes therein, wherein the sonicator or the plate is located between the piston and the side wall. The implantable device could further comprise a first chamber between the semipermeable plug and the piston, a second chamber between the piston and the sonicator or the plate, and a third chamber between the second chamber and the side wall, wherein the first chamber comprises a salt solution, the second chamber comprises the beads, and the third chamber comprises a foam. Preferably, the sonicator is configured to sonicate and increase porosity of the foam. Preferably, some or all of the holes in the plate are filled with a phase-change material. Preferably, the side wall contains the sensor.

Another embodiment relates to an implantable device comprising a pump that is configured to produce a flow rate of elution of a drug from the implantable device to be maintained substantially constant, increased or decreased with time, or even stopped momentarily; beads comprising a core and a shell, the core being enclosed by the shell, the core comprising the drug, and the shell comprising a first material and a second material; wherein the first material is distributed in the second material; wherein the first material is configured to create openings in the shell; wherein the openings allow the drug to be released from the core to the exterior of the shell through the openings.

Preferably, the first material comprises a metal-containing material that forms the openings in the shell or a first biodegradable material that degrades over time, and the second material comprises a second biodegradable material. Preferably, the metal-containing material is configured to form openings in the shell, before or after implanting or attaching the implantable device in or on the body of a subject, when the shell is exposed to an external stimulus.

In an aspect, a device is described. The device comprises a first chamber comprising a drug reservoir unit and an osmotic unit; a second chamber comprising an electronic unit; a third chamber comprising a drug delivery unit; and an electric valve. The implantable device is fully implanted in a subject during operation. The implantable device releases a drug into the subject at a flow rate such that a variation in the flow rate is within plus-minus 15% by volume.

In an embodiment, the device is configured to deliver a dynamic volume of the drug into the body of the subject. In another embodiment, the device is further configured to mimic a pattern of repeated injections.

In another embodiment, the device is configured to deliver a drug into the body of a subject.

In another embodiment, the device is configured to deliver a dynamic volume of the drug in response to at least one of a current state, and a current health condition of the subject. In another embodiment, the device is configured to deliver a consistent volume of the drug in response to at least one of a current state, and a current health condition of the subject.

In another embodiment, the device is configured to deliver a constant volume of the drug into a body of the subject. In another embodiment, variability per shot of the drug by the device is ±10% by volume. In another embodiment, variability per shot of the drug by the device is ±5% by volume. In another embodiment, variability per shot of the drug by the device is ±15% by volume.

In another embodiment, the device further comprises: a cybersecurity module; a communication module; and an artificial intelligence module. In another embodiment, the cybersecurity module utilizes a risk-based mitigation technique to overcome cybersecurity attacks.

In another embodiment, the cybersecurity module utilizing the risk-based mitigation technique creates at least one of an implantable medical device (IMD) security impact matrix, and an IMD access requirements matrix. In another embodiment, the IMD security impact matrix comprises one or more IMD data types, one or more delivering commands, and one or more impact scores.

In another embodiment, the one or more IMD data types comprises one of a confidentiality data type; an integrity data type; and an availability data type.

In another embodiment, the one or more delivering commands comprises one of emergency, invoke, and reset. In another embodiment, the one or more impact scores comprises one of LOW, MEDIUM, and HIGH. In another embodiment, the cybersecurity module estimates impact for one or more delivering commands and the one or more IMD data types and assigns an impact score of the one or more impact scores. In another embodiment, the IMD access requirement matrix comprises one or more roles, one or more delivering commands, and one or more access privileges.

In another embodiment, the one or more roles comprises a patient, a prescribing physician, a maintenance physician and an emergency technician, wherein the one or more roles request access to the device. In another embodiment, the one or more delivering commands comprises emergency, invoke, and reset. In another embodiment, one or more access privileges comprises READ, and WRITE. In another embodiment, the cybersecurity module utilizes one or more authentication mechanisms. In another embodiment, the one or more authentication mechanisms comprises a password authentication mechanism, a device-to-device handshake authentication mechanism, and a cryptographic authentication mechanism. In another embodiment, the cybersecurity module utilizes one or more alert mechanisms. In another embodiment, the one or more alert mechanisms comprises an audible alert mechanism and an automatic device state trigger mechanism.

In another embodiment, the automatic device state trigger mechanism triggers one of a safe state, an emergency state and a reset state. In another embodiment, the device-to-device handshake authentication mechanism is adapted to establish authentication between the device and one or more external devices. In another embodiment, the cybersecurity module tailors one or more authentication mechanisms based on at least one of device size, cost, power, computational capability, and storage. In another embodiment, the cybersecurity module adjusts one or more security mechanisms to accommodate within the device. In another embodiment, the cybersecurity module receives an encrypted key from an external device for authentication and to establish communication between the device and the external device. In another embodiment, the communication module is configured to communicate with at least one of one or more sensors, one or more external devices, one or more servers, and one or more databases.

In another embodiment, the one or more sensors comprises a temperature sensor, a pH sensor, a flow sensor, a pressure sensor, an electrocardiography (ECG) sensor, a valve sensor, and a global positioning system (GPS). In another embodiment, the communication module fetches information from at least one of one or more sensors, one or more external devices, one or more servers, and one or more databases to the device. In another embodiment, the communication module fetches information of at least one of an inductive pulse and a magnetic pulse from a battery charge coil to the device. In another embodiment, the communication module is a wireless communication module. In another embodiment, the communication module communicates through one of a near field communication. In another embodiment, the communication module communicates through one of a far field communication. In another embodiment, the communication module communicates a notification to one or more external devices.

In another embodiment, the notification comprises emergency message, a current medical condition, a sensor reading, a precaution instruction, a medication update, and/or an alert message. In another embodiment, the communication module communicates the notification to one or more external devices at a scheduled time. In another embodiment, the communication module communicates the notification to the one or more external devices upon receiving a command from the artificial intelligence module. In another embodiment, the communication module communicates the notification to the one or more external devices upon detecting when one or more events occurred. In another embodiment, the one or more events comprises a request from the one or more external devices, and a mechanical trigger from the one or more external devices. In another embodiment, the mechanical trigger comprises one of an influence of a magnetic field and an inductance on the device. In another embodiment, the artificial intelligence module is embedded within the device to perform computation locally within the device. In another embodiment, the artificial intelligence module performs the computation in real-time within the device. In another embodiment, the artificial intelligence module performs the computation autonomously in real-time. In another embodiment, the artificial intelligence module communicates with one or more external servers in order to perform complex computation. In another embodiment, the artificial intelligence module communicates with one or more external databases to extract information and perform computation locally within the device. In another embodiment, the artificial intelligence module communicates with one or more sensors to obtain readings and perform computation locally within the device. In another embodiment, the communication module comprises a printed antenna. In another embodiment, the artificial intelligence module performs the computation locally within the device within a predefined time period. In another embodiment, the artificial intelligence module performs the computation locally within the device enables to transmit externally only the relevant data ensuring data privacy.

In another embodiment, the artificial intelligence module performs the computation locally within the device enables data reduction on one or more external servers and one or more external devices. In another embodiment, the electric valve comprises one of a piezoelectric valve, an electromagnetic valve, a shape memory alloy valve, a diaphragm valve, a solenoid valve, an electrostatic valve, and a micro-fluidic valve. In another embodiment, the drug delivery unit comprises a plurality of drug delivery orifices. In another embodiment, the electric valve comprises a cartwheel structure to distribute one port outlet to many outlets on periphery of the device. In another embodiment, the device comprises the osmotic unit comprising a semi permeable membrane, an osmotic solution, and a plate; wherein the osmotic unit is configured to move forward in a direction of the electric valve after the injection. In another embodiment, the semi permeable membrane is configured for inflow of a body fluid into the osmotic unit. In another embodiment, a force on a first side of a plunger of the valve due to initial osmotic pressure is less than a force on a second side of the plunger.

In another embodiment, the semipermeable membrane has a substantially constant permeability; wherein the permeability rate is independent of concentration difference across the semipermeable membrane. In another embodiment, the electronic unit comprises a power component and a control component. In another embodiment, the control component is configured to control a delivery of the drug based on at least one of a pre-programmed delivery protocol and a real-time command received. In another aspect, a device comprises a first chamber comprising a drug reservoir unit and an osmotic unit; a second chamber comprising an electronic unit; a third chamber comprising a drug delivery unit; and an electric valve. The implantable device is fully implanted in a human or animal body during operation. The implantable device releases a drug into the human or animal body at a desired flow rate. The implantable device has a built-in failure detection mechanism to detect a failure in the implantable device. In another aspect, a device comprises a first chamber comprising a drug reservoir unit and an osmotic unit; a second chamber comprising an electronic unit; a third chamber comprising a drug delivery unit; and an electric valve. The implantable device is fully implanted in a human or animal body during operation. The implantable device releases a drug into the human or animal body at a flow rate such that the flow rate produces repeated shots of the drug into the human or animal body. In another embodiment, the osmotic unit comprises a semipermeable membrane. In another embodiment, the osmotic unit further comprises a hydrogel. In another embodiment, the osmotic unit further comprises a salt solution. In another embodiment, the semipermeable membrane is made of at least one of a polymethylmethacrylate, a polyurethane, a polyamide, a polyether-polyamide copolymer, a thermoplastic co-polyester.

In another embodiment, the osmotic unit is configured to move forward in the direction of the electronic valve. In another embodiment, the drug reservoir unit comprises the drug. In another embodiment, the drug reservoir unit is sized to hold a predefined amount of the drug. In another embodiment, the predefined amount of the drug can be configured based on a drug type, an amount of a drug needed for each dosage, number of doses per day, and number of days before a refill. In another embodiment, the drug reservoir unit is refillable. In another embodiment, a volume of the drug delivered is adjustable based on a suggested dosing requirement. In another embodiment, the electronic unit is configured to provide an electromagnetic force to the electric valve to push the drug to the drug delivery unit. In another embodiment, the electronic unit comprises an insulation cap. In another embodiment, the electronic unit comprises a power component and a control component. In another embodiment, the power component comprises an energy source, and a power switch. In another embodiment, the control component comprises a timer and a microcontroller, wherein the microcontroller is programmed using a wired or wireless connection. In another embodiment, the microcontroller is designed to control the delivery of the drug based on a pre-programmed delivery protocol. In another embodiment, the microcontroller is configured to control the delivery of the drug based on a real-time command received. In another embodiment, the microcontroller is configured to be wirelessly programmed to release the drug inside the body of the subject upon activation by a wireless signal. In another embodiment, the real-time command is received by wireless communication.

In another embodiment, inner circumference of the drug reservoir unit comprises a zip tie surface. In another embodiment, the device further comprises a plurality of seals. In another embodiment, the plurality of seals comprises at least one of a cone seal, a fluid pressure assisted seal, a spring assisted seal, an O-ring seal, and a ridge seal. In another embodiment, the plurality of seals comprises one of a Teflon and nylon. In another embodiment, the control component of the device is connected to an artificial intelligence and machine learning system via wireless communication. In another embodiment, the artificial intelligence and machine learning system determines a fit of an implant in the body. In another embodiment, the artificial intelligence and machine learning system determines one or more of a prognosticator, an indicator, and a risk factor of postoperative performance of the device implanted. In another embodiment, the artificial intelligence and machine learning system is configured to generate an optimized treatment experience for the subject. In another embodiment, the device further comprises a sonicator. In another embodiment, a control of a flow pattern of repeated injections is configured via the sonicator. In another embodiment, the device is configured to provide at least one of a constant drug concentration, an increasing drug concentration, and a decreasing drug concentration. In another embodiment, the device provides a constant drug concentration for a predefined time, no drug release for some time, and repeats the constant drug concentration for the predefined time. In another embodiment, the device delivers a consistent drug level by producing a drug release profile that is programmed ahead of time. In another embodiment, the device delivers a consistent drug level by producing a drug release profile that is implemented in real-time.

In another embodiment, a desired flow rate of elution of the drug is such that a maximum limit and a minimum limit of a concentration of the drug in a blood serum of the subject are Cₘₐₓ and Cₘᵢₙ, respectively, wherein Cₘₐₓ and Cₘᵢₙ are predetermined maximum limit and minimum limit of the concentration of the drug. In another embodiment, the Cₘᵢₙ and the Cₘₐₓ are 80% to 125% of the desired concentration of the drug in the body of the subject. In another embodiment, the Cₘᵢₙ and the Cₘₐₓ are such that a 90% confidence interval of a peak concentration of the drug in the blood serum of the subject versus a reference is within 80% to 125% of a desired concentration of the reference in the blood serum of the subject, wherein the reference is a product approved by United States Food and Drug Administration.

In another embodiment, an enclosure of the energy source is biocompatible. In another embodiment, the energy source comprises a battery. In another embodiment, the battery is a onetime use battery. In another embodiment, the one-time use battery comprises at least one of a Lithium battery, a nuclear battery, a lead-acid battery, a dry cell battery, a nickel-cadmium battery, and a fuel cell. In another embodiment, the battery is a chargeable battery. In another embodiment, the chargeable battery is charged by radiofrequency (RF) charging. In another embodiment, the chargeable battery is charged by optical charging mechanism. In another embodiment, the chargeable battery is charged by ultrasonic transducers. In another embodiment, the chargeable battery is charged by inductive coupling. In another embodiment, the chargeable battery is powered by environmental harvesting-based cells. In another embodiment, the environmental harvesting-based cells comprises one of a biofuel cell, a thermoelectricity-based cell, a piezoelectricity-based cell, an electrostatics-based cell, and an electromagnetics-based cell.

In another embodiment, the electronic unit further comprises an electromagnet and an electronic circuitry. In another embodiment, the device comprises one or more primary sensors. In another embodiment, the one or more primary sensors comprise a concentration sensor to measure a drug concentration in the body of the subject. In another embodiment, the one or more primary sensors comprise a pressure sensor that is configured to measure a pressure in one or more chambers of the device. In another embodiment, the device comprises a biodegradable polymer. In another embodiment, the biodegradable polymer comprises at least one of a thermoplastic aliphatic polyester, a poly(amide), a poly(anhydride), a poly(phosphazene), a poly(dioxanone). In another embodiment, the thermoplastic aliphatic polyester comprises at least one of a poly(lactic acid), a poly (glycolic acid), poly(lactic-co-glycolic acid) and poly(caprolactone). In another embodiment, the device comprises an implantable grade plastic material. In another embodiment, the implantable grade plastic material comprises at least one of a polyethylene, a polypropylene, an acrylonitrile, a polycarbonate, a polyurethane, a Polyether ether ketone (PEEK) and a moldable composite.

In another embodiment, the moldable composite comprises at least one of a hydroxylapatite and a polylactic acid. In another embodiment, the device comprises an implantable grade metal. In another embodiment, the device comprises an implantable grade metal alloy. In another embodiment, the subject is one of a human and an animal. In another embodiment, delivery of the consistent drug level by the device comprises a desired dosage cycle. In another embodiment, the drug comprises a targeting material or a targeting molecule that binds to at least one of a certain organ, a tissue, object, and a specific site within the body of the subject. In another embodiment, the drug is enclosed in a bead. In another embodiment, the bead comprises a core and a shell with the core being enclosed by the shell and the bead contains the drug. In another embodiment, the bead comprises a targeting material or a targeting molecule that binds to at least one of a certain organ, a tissue, an object, and a specific site within the body of the subject. In another embodiment, the shell comprises a stimuli-responsive polymer configured to break the shell, before or after implanting or attaching the device in or on the body of the subject, when the bead is exposed to an external stimulus. In another embodiment, the shell comprises a first material and a second material. In another embodiment, the first material comprises at least one of a metal-containing material and a first biodegradable material. In another embodiment, the second material comprises a second biodegradable material. In another embodiment, the first material is distributed in the second material. In another embodiment, the first material is configured to create pores in the second material. In another embodiment, the pores allow the drug to flow from within the bead to outside the bead.

In another embodiment, the metal-containing material is configured to form pores in the shell, before or after implanting or attaching the device in or on the body of the subject, when the bead is exposed to an external stimulus. In another embodiment, the metal-containing material comprises metallic particles. In another embodiment, the metallic particles comprise at least one of an iron-containing particle, a manganese-containing particles, an iron-containing polymer, and a manganese-containing polymer. In another embodiment, the first and second materials comprise polymers. In another embodiment, the first material comprises polylactic acid (PLA) or an iron-containing polymer and the second material comprises poly E-caprolactone (PCL). In another embodiment, the bead further contains a polymer. In another embodiment, the desired flow rate of elution of the drug is substantially constant. In another embodiment, a desired flow rate of elution of the drug is substantially constant for a first period of time and substantially zero for a second period of time, or vice versa. In another embodiment, a desired flow rate of elution of the drug is increasing or decreasing with time. In another embodiment, the electronics unit further comprises a chip comprising a cyber security module. In another embodiment, the device is disposable. In another embodiment, the device is reusable.

In another embodiment, the communication module is configured for communicating with remote devices. In another embodiment, the communication module comprises a magnetic induction device. In another embodiment, the communication module comprises an infra-red device. In another embodiment, the communication module comprises a radiofrequency device. In another embodiment, the drug reservoir unit is refillable.

In another aspect, the system comprises: a device comprising a first chamber comprising a drug reservoir unit and an osmotic unit; a second chamber comprising an electronic unit; a third chamber comprising a drug delivery unit; an electric valve; one or more primary sensors. The implantable device is configured to deliver a drug into the body of a subject. The device is implantable into the body of the subject. The one or more primary sensors are configured to monitor a bioactivity. The control component adjusts a dose of the drug based on the bioactivity.

In another embodiment, bioactivity is a biochemical activity. In another embodiment, the biochemical activity comprises a change in a metabolite concentration. In another embodiment, the biochemical activity comprises a change in concentration of a biomarker. In another embodiment, the biomarker comprises one or more of a diagnostic biomarker, a prognostic biomarker, a predictive biomarker, a surrogate biomarker, a monitoring biomarker, a pharmacodynamic biomarker and a safety biomarker. In another embodiment, the bioactivity is a biophysical activity. In another embodiment, the biophysical activity comprises a change in heart rate. In another embodiment, the biophysical activity comprises a change in blood pressure. In another embodiment, the biophysical activity comprises a change in body temperature. In another embodiment, the biophysical activity comprises a change in respiratory rate. In another embodiment, the biophysical activity comprises a change in oxygen saturation.

In yet another aspect, a system is described herein. The system comprises: a device comprising a first chamber comprising a drug reservoir unit and an osmotic unit; a second chamber comprising an electronic unit; a third chamber comprising a drug delivery unit; an electric valve; and one or more primary sensors. The implantable device is configured to deliver a drug into the body of a subject. The device is implantable into the body of the subject. The one or more primary sensors are configured to monitor an anomaly in the device. The implantable device is fully implanted in a subject during operation, and wherein the implantable device releases a drug into the subject at a flow rate such that a variation in the flow rate is within plus or minus 15% by volume. In another embodiment, the anomaly comprises one or more occlusions, a mechanical error, an irregular fluid flow, a drug reservoir empty condition, and an electronic failure. In another embodiment, the one or more primary sensors comprise one or more of a flow condition sensor, an occlusion sensor, a volume sensor, a MEMS sensor, a force sensor, a contact sensor, a position sensor, a pressure sensor, a displacement sensor, an inlet valve sensor, an outlet valve sensor, and an actuator sensors. In another embodiment, the one or more primary sensors are responsive to one of contact, pressure, light, magnetism, strain, and density. In another embodiment, one or more primary sensors is connected to the electronic unit via a switch. In another embodiment, the electronic unit comprises an alarm connected to the sensor. In another embodiment, the alarm comprises an audible alarm. In another embodiment, the alarm comprises a visual alarm. In another embodiment, the control component further comprises a processor connected to the sensor. In another embodiment, the system further comprises an alarm connected to the processor. In another embodiment, the alarm comprises an audible alarm. In another embodiment, the alarm comprises a visual alarm. In another embodiment, the processor is programmed to activate the alarm upon receiving the threshold signal from the sensor. In another embodiment, the processor is programmed to activate the alarm upon receiving the threshold signal from the sensor for more than a predetermined period. In another embodiment, the processor is programmed to activate the alarm upon receiving the threshold signal from the sensor for less than a predetermined period. In another embodiment, the sensor provides an analog signal, and the processor includes an analog-to-digital converter for converting the analog signal of the sensor into a digital signal. In another embodiment, the processor is programmed to provide a signal indicative of the anomaly upon receiving the threshold signal from the sensor. In another embodiment, the processor is programmed to provide a signal indicative of the anomaly upon receiving the threshold signal from the sensor for more than a predetermined period. In another embodiment, the processor is programmed to provide a signal indicative of the anomaly upon receiving the threshold signal from the sensor for less than a predetermined period. In another embodiment, the processor is programmed to receive the threshold signal from the sensor when the device is primed. In another embodiment, the processor comprises a watchdog timer capability. In another embodiment, the electronic unit comprises an antenna. In another embodiment, the antenna is a multiple-input-multiple-output (MIMO) antenna. In another embodiment, the antenna is configured for high-data-rate telemetry and for sensing an inner condition of the body.

In yet another aspect, a system comprises: a device comprising a first chamber comprising a drug reservoir unit and an osmotic unit; a second chamber comprising an electronic unit; a third chamber comprising a drug delivery unit; and an electric valve; one or more secondary sensor configured to monitor a bioactivity. The control component is configured to adjust a dose of the drug based on bioactivity. The implantable device is fully implanted in a subject during operation, and wherein the implantable device releases the drug into the subject at a flow rate such that a variation in the flow rate is within plus or minus 15% by volume. In another embodiment, the secondary sensor is inserted in the body of the subject at a location elsewhere from the implantable device. In another embodiment, the secondary sensor is a wearable sensor. In another embodiment, bioactivity is a biochemical activity. In another embodiment, the biochemical activity comprises a change in a metabolite concentration. In another embodiment, the biochemical activity comprises a change in concentration of a biomarker. In another embodiment, the biomarker comprises one or more of a diagnostic biomarker, a prognostic biomarker, a predictive biomarker, a surrogate biomarker, a monitoring biomarker, a pharmacodynamic biomarker and a safety biomarker. In another embodiment, the bioactivity is a biophysical activity. In another embodiment, the biophysical activity comprises a change in heart rate. In another embodiment, the biophysical activity comprises a change in blood pressure. In another embodiment, the biophysical activity comprises a change in body temperature. In another embodiment, the biophysical activity comprises a change in respiratory rate. In another embodiment, the biophysical activity comprises a change in oxygen saturation. In another aspect, the system comprises: a device comprising a first chamber comprising a drug reservoir unit and an osmotic unit; a second chamber comprising an electronic unit; a third chamber comprising a drug delivery unit; an electric valve; and an external housing comprising external charging circuit for wirelessly recharging the battery. The implantable device is fully implanted in a subject during operation, and wherein the implantable device releases a drug into the subject at a flow rate such that a variation in the flow rate is within plus or minus 15% by volume.

In another embodiment, the device comprises a secondary coil capable of receiving energy from the external charging circuit. In another embodiment, the external charging circuit comprises a primary coil capable of inductively energizing the secondary coil when the housing is externally placed in proximity of the secondary coil with a first surface of the housing positioned closest to the secondary coil. In another embodiment, the first surface of the external housing being a thermally conductive surface.

In yet another aspect, a system is described herein. The system comprises: a device comprising a first chamber comprising a drug reservoir unit and an osmotic unit; a second chamber comprising an electronic unit; a third chamber comprising a drug delivery unit; and an electric valve; an external housing comprising a transceiver capable of transmitting one or more transmission signals. The transceiver communicates with the implantable device. The implantable device is fully implanted in a subject during operation. The implantable device releases a drug into the subject at a flow rate such that a variation in the flow rate is within plus or minus 15% by volume.

In another embodiment, the transceiver powers the implantable device. In another embodiment, the transceiver is configured to receive one or more transmission signals from the implantable device. In another embodiment, the transceiver is configured to calculate one or more information based on the one or more transmission signals. In another embodiment, the transceiver is configured to generate one or more of a measurement trend, an alert, and an alarm based on the calculated one or more information.

In an aspect, the system comprising: a device comprising a first chamber comprising a drug reservoir unit and an osmotic unit; a second chamber comprising an electronic unit; a third chamber comprising a drug delivery unit; an electric valve; one or more secondary sensor configured to monitor a bioactivity; and an external housing comprising a thermoregulator. The thermoregulator is capable of thermo-heating or cooling the implantable device to maintain the implantable device at body temperature even if the body temperature fluctuates. The implantable device is fully implanted in a subject during operation, and wherein the implantable device releases a drug into the subject at a flow rate such that a variation in the flow rate is within plus or minus 15% by volume.

In another embodiment, the device comprises a cyber security module. In another embodiment, the cyber security module further comprises an information security management module providing isolation between the implantable device and the external processor. In another embodiment, the information security management module is operable to: receive data from at least one of the implantable device, the external processor, the secondary sensor, and the database; exchange a security key at a start of communication between a communication module and the external processor; receive the security key from the external processor; authenticate an identity of the external processor by verifying the security key; analyze the security key for a potential cyber security threat; negotiate an encryption key between the communication module and the external processor; encrypt the data; and transmit the encrypted data to the external processor when no cyber security threat is detected. In another embodiment, the information security management module is operable to: exchange a security key at a start of communication between a communication module and the external processor; receive the security key from the external processor; authenticate an identity of the external processor by verifying the security key; analyze the security key for a potential cyber security threat; negotiate an encryption key between the system and the external processor; receive encrypted data; decrypt the encrypted data; perform an integrity check of the decrypted data; and transmit the decrypted data to at least one of one of the implantable device, the external processor, the secondary sensor, and the database through the communication module when no cyber security threat is detected. In another embodiment, the information security management module is configured to raise an alarm when the cyber security threat is detected. In another embodiment, the information security management module is configured to discard the encrypted data received if the integrity check of the encrypted data fails. In another embodiment, the information security management module is configured to check the integrity of the encrypted data by checking accuracy, consistency, and any possible data loss during communication through the communication module. In another embodiment, the information security management module is configured to perform asynchronous authentication and validation of the communication between the communication module and the external processor. In another embodiment, the cybersecurity module comprises a perimeter network configured to provide an extra layer of protection. In another embodiment, the perimeter network is configured to protect the device from cyber security threat by using a plurality of firewalls.

In another embodiment, the device further comprises an artificial intelligence module. In another embodiment, the device is connected to an artificial intelligence module via a wireless communication module. In another embodiment, the artificial intelligence module determines suitability of the device in the body. In another embodiment, the artificial intelligence module determines one or more of a prognosticator, an indicator, and a risk factor of postoperative performance of the device implanted. In another embodiment, the artificial intelligence and machine learning system is configured to generate an optimized treatment experience for the subject. In another embodiment, the device is refillable and wherein the electronic circuitry is configured to determine a refill information based on the data and cause a communication module to send the refill information to an external device. In another embodiment, the communication module is wireless. In another embodiment, the artificial intelligence module comprises a machine learning model, wherein the machine learning model is configured to be trained. In another embodiment, the machine learning model is a neural network model. In another embodiment, the neural network model is a recurrent neural network model. In another embodiment, the artificial intelligence module is configured to train at least based on one of a Random Forest algorithm, a Bayesian network algorithm, a Support vector machine algorithm, and a penalized logistic regression algorithm. In another embodiment, the artificial intelligence module is configured to train a prediction component of the system. In another embodiment, the device comprises an artificial intelligence engine configured for at least one of a real-time monitoring of a patient's health data, an adjustment in drug dosage, and identifying potential faults in the device. In another embodiment, the artificial intelligence engine is configured to monitor a current health condition of the patient in real-time and in response to the current health condition a drug dosage is altered dynamically in real-time for the patient. In another embodiment, the artificial intelligence engine is configured to reside in the device. In another embodiment, the artificial intelligence engine is configured to reside in a cloud network. In another embodiment, the artificial intelligence engine is configured to detect an anomaly in the device, wherein the anomaly comprises at least one of a sensor failure, a device failure, a severe reaction in the patient, a communication failure between the components of the device, a battery power drain, a high battery voltage, a low battery voltage, a transmission failure, a reception failure, and an internet connectivity failure.

In another embodiment, the artificial intelligence engine is configured to detect the sensor failure by monitoring data received from the sensors. In another embodiment, the artificial intelligence engine is configured to detect the sensor failure by monitoring at least one of a no data from the sensors, bad data from the sensors, and irrelevant data from the sensors. In another embodiment, the artificial intelligence engine is configured to monitor plurality of sensors associated with the device, wherein the plurality of sensors comprise plurality of first sensors of the implantable device, plurality of second sensors associated with an external device, plurality of third sensors affixed to the patient's body, plurality of fourth sensors implanted in the patient's body, plurality of fifth sensors that are in communication with the implantable device. In another embodiment, the artificial intelligence engine is configured to train an artificial intelligence model with data sets from the plurality of sensors for one or more scenarios. In another embodiment, the one or more scenarios comprise at least one of a diabetes, a high blood pressure, a low blood pressure, a brain tumor, a cancer. In another embodiment, the artificial intelligence model is trained via at least one of a supervised learning and an unsupervised learning. In another embodiment, the artificial intelligence engine is configured to send test signals to detect health of the system. In another embodiment, the artificial intelligence engine is configured for an adjustment in drug dosage.

In another embodiment, the adjustment in drug dosage is dynamic and is based on at least one of a current health condition of the patient, a current state of the patient, and demographics information of the patient; wherein the demographics information of the patient comprises at least one of a name, contact information, birthdate, age, gender and pronouns, allergies, languages spoken and preferred language, relationship status, previous medical history, ethnicity, race, occupation, and resuscitation status; and wherein the current state of the patient is at least one of a sleeping state, a resting state, an exercising state, a running state, a swimming state, an awake state. In another embodiment, the current state of the patient is determined based on one or more sensors. In another embodiment, the artificial intelligence engine is configured to consider feedback from a patient or a caregiver, wherein the feedback is taken via at least one of a user interface of a computing device, a sensor reading, a camera capturing the patient, and a video capturing the patient. In another embodiment, the adjustment in drug dosage is based on a profile of the patient. In another embodiment, the profile of the patient comprises at least one of a current medical record, a medication prescribed, a test report, and a historic medical data of the patient. In another embodiment, the artificial intelligence engine is configured to determine a boundary of the patient based on at least one of a cluster, a category, and a cohort.

In another embodiment, the artificial intelligence engine is configured to undergo training in at least one of a first cohort comprising at least one of a race, an ethnicity, a medical condition and a second cohort comprising at least one of a specific medical condition to the patient. In another embodiment, the artificial intelligence engine is configured to recommend a pharmaceutical product for the patient to treat a medical condition. In another embodiment, the artificial intelligence engine is configured to finetune an artificial intelligence model specifically for the patient. In another embodiment, the artificial intelligence engine is configured to train an artificial intelligence model via a supervised learning when the patient is using the device. In another embodiment, the artificial intelligence engine is configured to determine a current state of the patient, a current health condition of the patient, a medical condition of the patient and dynamically alter at least one of the drug and a dosage. In another embodiment, the artificial intelligence engine is configured to have a feedback loop, wherein at least of an input from the patient, a sensor data, a data from external device, a symptom occurring post dosage of the drug, are considered into the feedback loop. In another embodiment, the artificial intelligence engine is configured to consider time series data with a time stamp. In another embodiment, the artificial intelligence engine is trained based on different ranges of values obtained from different data sources and different datasets received from different types of devices. In another embodiment, the artificial intelligence engine is configured to adjust for device-to-device variations. In another embodiment, the artificial intelligence engine is configured to adjust for sensor drift. In another embodiment, the artificial intelligence engine is configured to determine a lag between the device data and the patient data. In another embodiment, the artificial intelligence engine is configured to adjust for a lag between the device data and the patient data, wherein the artificial intelligence engine is pretrained for an adjustment factor. In another embodiment, the adjustment factor depends on the current state of the user, wherein the current state is at least one of an exercising state, a rest state, a sleep state, a wake up state, an idle state. In another embodiment, the adjustment in drug dosage is an amount of the drug or a time of dosage of the drug. In another embodiment, the artificial intelligence engine is configured to detect drifting in a measured data of the patient in the wrong direction in a first spike as early as possible leading to reschedule and re-adjust the medication. In another embodiment, the device is coupled to a receiving unit and an external unit, wherein the external unit comprises sensors external to the device. In another embodiment, the artificial intelligence engine is configured to train on the fly with the real-time data of the patient, of the device and external sensor data. In another embodiment, the artificial intelligence engine is configured to provide one or more alerts, wherein the one or more alerts are further categorized into one of a high alert, a medium alert, and a low alert. In another embodiment, the artificial intelligence engine is configured to learn from a wrongly categorized alert via supervised training. In another embodiment, the artificial intelligence engine is configured to learn and minimize false positives and false negatives. In another embodiment, an operator monitors one or more alerts and their categorization for correctness and provides a correctly labeled for training an artificial intelligence model. In another embodiment, the artificial intelligence engine is configured to learn in unsupervised learning mode and classify the data into clusters and refine them over a period of time upon continuous monitoring. In another embodiment, the artificial intelligence engine is configured to take a voice input from a voice module of an audio device, wherein the voice input is configured as feedback of the patient to the device. In another embodiment, the audio device comprises a voice input module and a voice output module, wherein the voice output module is configured to ask one or more questions to collect feedback from the patient. In another embodiment, the artificial intelligence engine is configured to pre-train an artificial intelligence model, wherein the pre-training is based on various categories comprising one or more of kids, men, women, boys, girls, older people. In another embodiment, the artificial intelligence engine is configured to train an artificial intelligence model, wherein the training comprises one or more of supervised learning, an unsupervised learning, a deep network, and a large language model. In another embodiment, the artificial intelligence engine is configured to determine a correlation between one or more datasets. In another embodiment, the artificial intelligence engine is configured to consider the special medical condition of the patient, wherein the special medical condition is one or more of pregnancy, a heart condition, a health anomaly, a congenital health condition, a health disorder. In another embodiment, the artificial intelligence engine is configured to monitor the patient before the dosage of the drug and after the dosage of the drug. In another embodiment, the artificial intelligence engine is configured to consider a time series data. In another embodiment, the artificial intelligence engine is configured to function when a data point or data series is missing and no anomaly is detected. In another embodiment, the artificial intelligence engine is configured to customize the dosage of the drug to the patient based on a recommendation of a physician. In another embodiment, the artificial intelligence engine is configured to automatically choose the dosage of the drug. In another embodiment, the artificial intelligence engine is configured to at least one of a determination and learning of interactions of one or more drugs. In another embodiment, the artificial intelligence engine is configured to a dosage requirement based on a medical database through Natural Language Processing. In another embodiment, the artificial intelligence engine is configured for embedded intelligence when there is a disruption in the connection. In another embodiment, the artificial intelligence engine is configured to protect the data using a cybersecurity module of the device.

In another embodiment, the osmotic unit comprises an osmotic pump to provide substantially zero-order release rates of a desired therapeutic agent. In another embodiment, the osmotic unit comprises an osmotic pump to provide pseudo zero-order flux (flow) of the solvent through the semipermeable membrane. In another embodiment, a constant flux of the solvent in the osmotic unit is maintained by varying a permeability of a semipermeable membrane such as increasing the permeability of the semipermeable membrane with the decrease in the difference of concentration across the semipermeable membrane.

In another embodiment, the processor is programmed to sleep most of time once an initial initialization of at least one of peripherals and Timers and a Real Time Clock (RTC) is complete. In another embodiment, the processor is woken up only when at least one of timers and the RTC wakes up the processor, and a watchdog timer times out. In another embodiment, the timers and the RTC wakes up the processor when at least one of a timer counter reaches the predefined time of the drug delivery, at an instant time to read the sensor data, and when communication module is to be activated to receive command and data from an external monitoring device. In another embodiment, the processor is programmed to enable at least one of deliver the drug in a predefined time period and acquire sensor reading in the predefined time period. In another embodiment, the processor is programmed to open the electric valve for a predefined duration to deliver a predefined quantity of the drug. In another embodiment, the electric valve is a latching solenoid valve that remains open until an electrical charge of an opposite polarity is applied to close the electric valve. In another embodiment, the electronic unit supplies a predefined pulse of the electrical charge to open or close the electric valve. In another embodiment, the processor monitors an open state duration of the electric valve and determines a predefined value of amount of dosage delivered. In another embodiment, the processor determines the predefined value of the amount of dosage delivered based on determining a distance travelled by the piston. In another embodiment, the processor scans the sensor data, reads the sensors, analyses the data, and stores the data. In another embodiment, the processor dynamically adjusts the dose based on the data received from the sensors. In another embodiment, the processor switched back to sleep state upon completing the task. In another embodiment, the processor performs a task to recover from a fault condition when the sensor readings indicate pre fault or the fault condition. In another embodiment, the processor activates a communication module and sends an emergency message to an external monitoring device. In another embodiment, the processor activates the communication module and performs a scheduled transmission of the sensor readings and scans for at least one of a message, and a command from the external monitoring device.

In another embodiment, the processor is programmed with a predefined timer interval for a sensor reading, and a communication by taking into account battery consumption at different states. In another embodiment, a battery supplies power to the processor. In another embodiment, a battery supplies a predefined electric pulse as the power to the processor. In another embodiment, the device comprises a communication module. In another embodiment, the communication module is a wireless communication module. In another embodiment, the communication module utilizes a short-range communication for transmitting and receiving data from the device. In another embodiment, the short-range communication operates at radio frequency from 402 to 405 MHz. In another embodiment, the device comprises a microcontroller interfaced with a Medical Implant Communication System (MICS) band transceiver. In another embodiment, the device bi-directionally communicates with a base station outside the human body within a short range. In another embodiment, the base station comprises a transceiver and built-in intelligence engine to communicate data. In another embodiment, the device comprises a communication technology module that is adapted to exchange data over internet. In another embodiment, the base station comprises a global system for mobile communication (GSM) module that enables transfer of the data over a cellular network. In another embodiment, the MICS band transceiver communicates the data over MICS Radio Frequencies to a base station. In another embodiment, the communication is performed over a physical layer communication. In another embodiment, the communication module comprises a data communication protocol, error handling protocol, and data security measures. In another embodiment, the base station is a standalone system based on the microprocessor or a microcontroller. In another embodiment, the communication module comprises a backscatter communication module. In another embodiment, the backscatter communication module uses an incident radiofrequency (RF) signal to transmit data without a battery or power source. In another embodiment, the backscatter communication module employs passive reflection and modulation of an incoming RF signal and converts it into electricity adapted to be encoded for data communication.

In another embodiment, the device comprises a data source, and a non-magnetic resonant antenna for backscatter communication with an external communication system. In another embodiment, the non-magnetic resonant antenna is printed on a surface of the device. In another embodiment, the non-magnetic resonant antenna comprises resonance absence. In another embodiment, the external communication system is configured to transmit electromagnetic waves towards the device and to receive backscattered signals from the device. In another embodiment, the external communication system is a transmit antenna and a receive antenna. In another embodiment, the transmit antenna and the receive antenna is used separately to reduce a coupling between the transmit antenna and the receive antenna to avoid receiver saturation. In another embodiment, the receive antenna operates over a predefined dynamic range to receive weak backscattered signals. In another embodiment, the communication module is configured for performing one to one communication. In another embodiment, the communication module is configured for performing one to many communications and many to many communications via a mesh network communication without needing an external network. In another embodiment, the system is configured to monitor the device from a remote place. In another embodiment, the device comprises a telemetry transceiver that transmits data to an external device. In another embodiment, the device communicates with the external device using a network compatible wireless link which minimizes a current drain. In another embodiment, the device schedules a periodic interrogation of the data to minimize the current drain. In another embodiment, the external device comprises a smart link device. In another embodiment, the backscatter communication module indicates a frequency for efficient charging.

In another embodiment, an external transmitter of the device adjusts the frequency for wireless power transmission based on feedback from the backscatter communication module. In another embodiment, the system comprises a wireless power transfer module that uses one or more inductive links for battery charging. In another embodiment, the microcontroller is in the form of a die on a flexible circuit substrate form which has a low sleep current. In another embodiment, the microcontroller comprises an internal timer.

An embodiment relates to a device comprising: a first chamber comprising a drug reservoir unit; a second chamber comprising an electronic unit; a third chamber comprising a drug delivery unit; and a displacement unit comprising an electromagnetic actuator, wherein the electromagnetic actuator comprises a piston a solenoid and a spring; and wherein the device is configured to deliver a drug into a body of a subject; wherein the device is implantable into the body of the subject; and wherein the displacement unit is non retractable and is configured to move only in one direction.

Another embodiment relates to a device comprising: a first chamber comprising a drug reservoir unit; a second chamber comprising an electronic unit; a third chamber comprising a drug delivery unit; and a displacement unit comprising an electromagnetic actuator, the electromagnetic actuator comprising a piston a solenoid and a spring; and wherein the device is fully implanted in a human or animal body during operation, and wherein the device releases a drug into the human or animal body at a flow rate such that a variation in the flow rate is within plus or minus 15% by volume.

Yet another embodiment relates to a device comprising: a first chamber comprising a drug reservoir unit; a second chamber comprising an electronic unit; a third chamber comprising a drug delivery unit; and a displacement unit comprising an electromagnetic actuator, the electromagnetic actuator comprising a piston a solenoid and a spring; and wherein the device is fully implanted in a human or animal body during operation, and wherein the device releases a drug into the human or animal body at a flow rate such that the flow rate produces repeated shots of the drug into the human or animal body.

Yet another embodiment relates to a device comprising: a first chamber comprising a drug reservoir unit; a second chamber comprising an electronic unit; a third chamber comprising a drug delivery unit; and a displacement unit comprising an electromagnetic actuator, the electromagnetic actuator comprising a piston a solenoid and a spring; and wherein the device releases a drug into the human or animal body at a desired flow rate, and wherein the device has a built-in failure detection mechanism to detect a failure in the device.

Yet another embodiment relates to a device comprising: a first chamber comprising a drug reservoir unit; a second chamber comprising an electronic unit; a third chamber comprising a drug delivery unit; and a displacement unit comprising an electromagnetic actuator, the electromagnetic actuator comprising a piston a solenoid and a spring; and wherein the device is configured to deliver a drug into a body of a subject; and wherein, when the device is implanted in the body of the subject, utilizes one of a spring force, a solenoid force, and a combination of the spring force and the solenoid force to move the drug from the drug reservoir unit into the body of the subject.

Yet another embodiment relates to a device comprising: a first chamber comprising a drug reservoir unit; a second chamber comprising an electronic unit; a third chamber comprising a drug delivery unit; and a displacement unit comprising an electromagnetic actuator, the electromagnetic actuator comprising a piston a solenoid and a spring; and wherein the device is configured to deliver a drug into a body of a subject; wherein the device is implantable into the body of the subject; and wherein the displacement unit is non retractable and is configured to move only in one direction.

Yet another embodiment relates to a device comprising: a first chamber comprising a drug reservoir unit; a second chamber comprising an electronic unit; a third chamber comprising a drug delivery unit; and a displacement unit comprising an electromagnetic actuator, the electromagnetic actuator comprising a piston a solenoid and a spring; and wherein the device is configured to deliver a drug into a body of a subject; wherein the device is implantable into the body of the body of the subject; and wherein the device is configured to mimic a flow pattern of repeated injections to deliver a consistent volume of the drug during each injection.

An embodiment relates to a method of making a device comprising a first chamber comprising a drug reservoir unit; a second chamber comprising an electronic unit; a third chamber comprising a drug delivery unit; and a displacement unit comprising an electromagnetic actuator, the electromagnetic actuator comprising a piston a solenoid and a spring and the method comprising: analyzing a three-dimensional data of the device to be printed; generating at least two printing paths on each layer of the device to be printed; providing a polymeric material; supplying the polymeric material to a 3D printing device to dispense the polymeric material in a layer-by-layer manner to create the device; and printing, via a printing nozzle of the 3D printing device, the device on a printing platform of the 3D printing device using the printing paths generated on each layer of, so that at least two nozzles are respectively moved along the corresponding printing paths.

An embodiment relates to system comprising: an implantable device comprising a first chamber comprising a drug reservoir unit; a second chamber comprising an electronic unit comprising a control component, a third chamber comprising a drug delivery unit; a displacement unit comprising an electromagnetic actuator, the electromagnetic actuator comprising a piston a solenoid and a spring; and one or more primary sensors; and wherein the implantable device is configured to deliver a drug into a body of a subject; wherein the device is implantable into the body of the subject; wherein the one or more primary sensor is configured to monitor a bioactivity; and wherein the control component adjusts a dose of the drug based on the bioactivity.

Another embodiment relates a system comprising: an implantable device comprising a first chamber comprising a drug reservoir unit; a second chamber comprising an electronic unit comprising a control component, a third chamber comprising a drug delivery unit; a displacement unit comprising an electromagnetic actuator, the electromagnetic actuator comprising a piston a solenoid and a spring; and one or more primary sensors; and wherein the implantable device is configured to deliver a drug into a body of a subject; wherein the device is implantable into the body of the subject; wherein the one or more primary sensor is configured to monitor an anomaly in the device.

Yet another embodiment relates to a system comprising: a device comprising a first chamber comprising a drug reservoir unit; a second chamber comprising an electronic unit comprising a control component, a third chamber comprising a drug delivery unit; a displacement unit comprising an electromagnetic actuator, the electromagnetic actuator comprising a piston a solenoid and a spring; and one or more secondary sensors configured to monitor a bioactivity; and wherein the control component is configured to adjust a dose of the drug based on the bioactivity.

Yet another embodiment relates to a device system comprising: a device comprising a first chamber comprising a drug reservoir unit; a second chamber comprising an electronic unit comprising a battery, a third chamber comprising a drug delivery unit; a displacement unit comprising an electromagnetic actuator, the electromagnetic actuator comprising a piston a solenoid and a spring; and an external housing comprising an external charging circuit for wirelessly recharging the battery.

Yet another embodiment relates to a device system comprising: an implantable device comprising a first chamber comprising a drug reservoir unit; a second chamber comprising an electronic unit comprising a battery, a third chamber comprising a drug delivery unit; a displacement unit comprising an electromagnetic actuator, the electromagnetic actuator comprising a piston a solenoid and a spring; and an external housing comprising a transceiver capable of transmitting one or more transmission signals; and wherein the transceiver communicates with the implantable device.

Yet another embodiment relates to a device system comprising: an implantable device comprising a first chamber comprising a drug reservoir unit; a second chamber comprising an electronic unit comprising a battery, a third chamber comprising a drug delivery unit; a displacement unit comprising an electromagnetic actuator, the electromagnetic actuator comprising a piston a solenoid and a spring; one or more secondary sensors configured to monitor a bioactivity; and an external housing comprising a thermoregulator; and

According to an embodiment, it is a device comprising a first chamber comprising a drug reservoir unit; a second chamber comprising an electronic unit; a third chamber comprising a drug delivery unit and a pump; and wherein the device is configured to deliver a drug into body of a subject; wherein the device is implantable into the body of the subject; and wherein the device is configured to deliver the drug in a controlled manner.

According to an embodiment it is a system comprising a device comprising, a first chamber comprising a drug reservoir unit; a second chamber comprising an electronic unit comprising a control component; a third chamber comprising a drug delivery unit and a pump; one or more primary sensors; and wherein the device is configured to deliver a drug into body of a subject; wherein the device is implantable into the body of the subject; wherein the device is configured to deliver the drug in a controlled manner; wherein the one or more primary sensor is configured to monitor a bioactivity; and wherein the control component adjusts a dose of the drug based on the bioactivity.

According to an embodiment it is a system comprising: a device comprising, a first chamber comprising a drug reservoir unit; a second chamber comprising an electronic unit comprising a control component; a third chamber comprising a drug delivery unit and a pump; one or more primary sensors; and wherein the device is configured to deliver a drug into body of a subject; wherein the device is implantable into the body of the subject; wherein the device is configured to deliver the drug in a controlled manner; wherein the one or more primary sensor is configured to monitor an anomaly in the device.

According to an embodiment it is a system comprising: a device comprising, a first chamber comprising a drug reservoir unit; a second chamber comprising an electronic unit comprising a control component; a third chamber comprising a drug delivery unit and a pump; one or more secondary sensors configured to monitor a bioactivity; and wherein the control component is configured to adjust a dose of the drug based on the bioactivity.

According to an embodiment it is a system comprising: a device comprising, a first chamber comprising a drug reservoir unit; a second chamber comprising an electronic unit comprising a control component; a third chamber comprising a drug delivery unit and a pump; an external housing comprising an external charging circuit for wirelessly recharging the battery.

According to an embodiment it is a system comprising: a device comprising, a first chamber comprising a drug reservoir unit; a second chamber comprising an electronic unit comprising a control component; a third chamber comprising a drug delivery unit and a pump; an external housing comprising a transceiver capable of transmitting one or more transmission signals; and wherein the transceiver communicates with the device.

According to an embodiment it is a system comprising: a device comprising, a first chamber comprising a drug reservoir unit; a second chamber comprising an electronic unit comprising a control component; a third chamber comprising a drug delivery unit and a pump; one or more secondary sensor configured to monitor a bioactivity; and an external housing comprising a thermoregulator; and wherein the thermoregulator is capable of thermo-heating or cooling the implantable device to maintain the implantable device at body temperature even if the body temperature fluctuates.

According to an embodiment of the device comprising: a first chamber comprising a drug reservoir unit; a second chamber comprising an electronic unit; a third chamber comprising a drug delivery unit and a pump; and wherein the device is configured to deliver a drug into body of a subject; wherein the device is implantable into the body of the subject; and wherein the device is configured to deliver the drug in a controlled manner.

According to an embodiment it is a system comprising: a device comprising, a first chamber comprising a drug reservoir unit; a second chamber comprising an electronic unit comprising a control component; a third chamber comprising a drug delivery unit and a pump; and one or more sensors coupled with the device configured for generating real-time data; a piston position module, wherein the piston position module is configured to measure a current position of the piston in real-time; an artificial intelligence module integrated with the device configured for at least one of a real-time monitoring health data of a user, an adjustment in drug dosage, and identifying potential faults in the device one or more primary sensors; one or more external sensors configured to generate data related to one or more of a health parameter, and a current state of the user; and a communication module integrated with the device configured to at least one of receive and transmit a data; a control module coupled with the device with the device wherein the control module is configured to adjust the drug dosage via a closed loop control, wherein the closed loop control is based on the a desired state of the patient and the current state of the patient; a patient input module, wherein the patient input module is configured to take an input from the patient; and wherein the device is implantable into the body of the subject; and wherein the device is configured to deliver the drug in a controlled manner.

According to an embodiment, there is a device comprising: a first chamber comprising a drug reservoir unit and an osmotic unit; a second chamber comprising an electronic unit; a third chamber comprising a drug delivery unit; and an electric valve; and wherein the device is fully implanted in a subject during operation, and wherein the implantable device releases a drug into the subject at a flow rate such that a variation in the flow rate is within plus or minus 15% by volume. In some embodiments, the device is configured to deliver a dynamic volume of the drug into a body of the subject. In some embodiments, the device is further configured to mimic a pattern of repeated injections. In some embodiments, the device is configured to deliver a drug into the body of a subject. In some embodiments, the device is configured to deliver a dynamic volume of the drug in response to at least one of a current state, and a current health condition of the subject. In some embodiments, the device is configured to deliver a consistent volume of the drug in response to at least one of a current state, and a current health condition of the subject. In some embodiments, the device is configured to deliver a constant volume of the drug into a body of the subject. In some embodiments, variability per shot of the drug by the device is ±10% by volume. In some embodiments, variability per shot of the drug by the device is ±5% by volume. In some embodiments, variability per shot of the drug by the device is ±15% by volume. According to an embodiment, there is a device comprising: a first chamber comprising a drug reservoir unit; a second chamber comprising an electronic unit; a third chamber comprising a drug delivery unit and a pump; and wherein the device is configured to deliver a drug into body of a subject; wherein the device is implantable into the body of the subject; and wherein the device is configured to deliver the drug in a controlled manner. In some embodiments, the device is configured to deliver a consistent volume of the drug during an injection; and wherein the device is further configured to mimic a pattern of repeated injections. In some embodiments, the pump is a positive displacement pump, wherein a pump chamber is filled and then emptied by an action of the pump. In some embodiments, the pump is at least one of a piezoelectric pump, an electromagnetic pump, electro-strictive micropump, peristaltic micropump, a microfluidic pump, a micro diaphragm pump, and a valveless micropump. In some embodiments, the pump comprises a valve, wherein the valve is at least one of a non-active valve and an active valve.

According to an embodiment, there is a device comprising: a first chamber comprising a drug reservoir unit; a second chamber comprising an electronic unit; a third chamber comprising a drug delivery unit; and a displacement unit comprising an electromagnetic actuator, wherein the electromagnetic actuator comprises a piston a solenoid and a spring; and wherein the device is configured to deliver a drug into a body of a subject; wherein the device is implantable into the body of the subject; and wherein the displacement unit is non retractable and is configured to move only in one direction.

In some embodiments, the displacement unit is configured to move towards the drug delivery unit by a predefined distance to release a predefined volume of the drug in the body of the subject.

In some embodiments, the device comprises one or more entrance holes distributed radially across a space created between the displacement unit and the electronic unit due to movement of the displacement unit, and wherein the one or more entrance holes are configured for an inflow of interstitial body fluids into the device.

In some embodiments, inner surface of the drug reservoir unit comprises a zip tie surface that is configured to prevent the displacement unit from retracting and to move only in one direction.

In some embodiments, the device comprises one or more primary sensors wherein the one or more primary sensors comprise a concentration sensor to measure a drug concentration in the body of the subject.

In some embodiments, the device comprises one or more secondary sensors wherein the one or more primary sensors comprise a concentration sensor to measure a drug concentration in the body of the subject.

In some embodiments, the device comprises one or more primary sensors wherein the one or more primary sensors comprise a diseases marker sensor.

In some embodiments, the device comprises one or more secondary sensors wherein the one or more primary sensors comprise a diseases marker sensor.

An embodiment relates to a device. The device comprises a first chamber comprising a drug reservoir unit and an osmotic unit; a second chamber comprising an electronic unit; a third chamber comprising a drug delivery unit; a displacement unit comprising an electromagnetic actuator; and wherein the device is configured to deliver a drug into body of a subject; wherein the device is implantable into the body of the subject; and wherein the device is configured to mimic a flow pattern of repeated injections to deliver a constant volume of the drug during each injection of the repeated injections.

In an embodiment of the device, the osmotic unit comprises a semipermeable membrane. In another embodiment of the device, the osmotic unit further comprises a hydrogel. In yet another embodiment of the device, the osmotic unit further comprises a salt solution. In yet another embodiment of the device, the semipermeable membrane is made of at least one of a polymethylmethacrylate, a polyurethane, a polyamide, a polyether-polyamide copolymer, a thermoplastic co-polyester. In yet another embodiment of the device, the drug reservoir unit comprises a sliding plate. In yet another embodiment of the device, the sliding plate is configured to move forward in a direction of the displacement unit. In yet another embodiment of the device, the sliding plate is micro holed for flow of the drug. In yet another embodiment of the device, the drug reservoir unit comprises the drug. In yet another embodiment of the device, the drug reservoir unit is sized to hold a predefined amount of the drug. In yet another embodiment of the device, the predefined amount of the drug can be configured based on a drug type, an amount of a drug needed for each dosage, number of doses per day, and number of days before a refill. In yet another embodiment of the device, the drug reservoir unit is refillable. In yet another embodiment of the device, the displacement unit comprises a plate having holes for flow of the drug from the drug reservoir unit to the drug delivery unit. In yet another embodiment of the device, the displacement unit comprises a piston and a spring. In yet another embodiment of the device, the constant volume of the drug delivered is adjustable based on a suggested dosing requirement. In yet another embodiment of the device, the electronic unit is configured to provide an electromagnetic force to the displacement unit to push the drug to the drug delivery unit. In yet another embodiment of the device, the drug delivery unit comprises a valve and an orifice. In yet another embodiment of the device, the valve is a one-way valve. In yet another embodiment of the device, the one-way valve comprises one of an umbrella valve, a duckbill valve, and a flapper valve. In yet another embodiment of the device, the one-way valve is an umbrella valve. In yet another embodiment of the device, the orifice comprises a plurality of orifices. In yet another embodiment of the device, the plurality of orifices are radially distributed around the drug delivery unit. In yet another embodiment of the device, size, and number of the plurality of orifices are configured based on structure of the device, material of the device, quantity of the drug to be delivered, and the rate at which the drug is to be delivered. In yet another embodiment of the device, the electronic unit comprises an insulation cap. In yet another embodiment of the device, the electronic unit comprises a power component and a control component. In yet another embodiment of the device, the power component comprises an energy source, and a power switch. In yet another embodiment of the device, the control component comprises a timer and a microcontroller. In yet another embodiment of the device, the microcontroller is programmed using a wired or wireless connection. In yet another embodiment of the device, the microcontroller is designed to control the delivery of the drug based on a pre-programmed delivery protocol. In yet another embodiment of the device, the microcontroller is configured to control a delivery of the drug based on a real-time command received. In yet another embodiment of the device, the microcontroller is configured to be wirelessly programmed to release the drug inside the body of the subject upon activation by a wireless signal. In yet another embodiment of the device, the real-time command is received by wireless communication. In yet another embodiment of the device, inner circumference of the drug reservoir unit comprises a zip tie like surface. In yet another embodiment of the device, the spring is a compression spring. In yet another embodiment of the device, the chargeable battery is charged by inductive coupling. In yet another embodiment of the device, the chargeable battery is powered by environmental harvesting-based cells. In yet another embodiment of the device, the environmental harvesting-based cells comprises one of a biofuel cell, a thermoelectricity-based cell, a piezoelectricity-based cell, an electrostatics-based cell, and an electromagnetics-based cell. In yet another embodiment of the device, the electronic unit further comprises an electromagnet and electronic circuitry.

In yet another embodiment of the device, the device comprises one or more primary sensors. In yet another embodiment of the device, one or more primary sensors comprise a concentration sensor to measure a drug concentration in the body of the subject. In yet another embodiment of the device, the one or more primary sensors comprise a pressure sensor that is configured to measure a pressure in one or more chambers of the device. In yet another embodiment of the device, the device comprises a biodegradable polymer. In yet another embodiment of the device, the biodegradable polymer comprises at least one of a thermoplastic aliphatic polyester, a poly(amide), a poly(anhydride), a poly(phosphazene), a poly(dioxanone). In yet another embodiment of the device, the thermoplastic aliphatic polyester comprises at least one of a poly(lactic acid), a poly (glycolic acid), poly(lactic-co-glycolic acid) and poly(caprolactone). In yet another embodiment of the device, the device comprises an implantable grade plastic material. In yet another embodiment of the device, the implantable grade plastic material comprises at least one of a polyethylene, a polypropylene, an acrylonitrile, a polycarbonate, a polyurethane, a Polyether ether ketone (PEEK) and a moldable composite. In yet another embodiment of the device, the moldable composite comprises at least one of a hydroxylapatite and a polylactic acid. In yet another embodiment of the device, the device comprises an implantable grade metal. In yet another embodiment of the device, the device comprises an implantable grade metal alloy. In yet another embodiment, the subject is one of a human and an animal. In yet another embodiment of the device, delivery of the consistent drug level by the device comprises a desired dosage cycle. In yet another embodiment of the device, the drug comprises a targeting material or a targeting molecule that binds to at least one of a certain organ, a tissue, object, and a specific site within the body of the subject. In yet another embodiment of the device, the drug is enclosed in a bead. In yet another embodiment of the device, the bead comprises a core and a shell with the core being enclosed by the shell and the bead contains the drug. In yet another embodiment of the device, the bead comprises a targeting material or a targeting molecule that binds to at least one of a certain organ, a tissue, an object, and a specific site within the body of the subject. In yet another embodiment of the device, the shell comprises a stimuli-responsive polymer configured to break the shell, before or after implanting or attaching the device in or on the body of the subject, when the bead is exposed to an external stimulus. In yet another embodiment of the device, the shell comprises a first material and a second material. In yet another embodiment of the device, the first material comprises at least one of a metal-containing material and a first biodegradable material. In yet another embodiment of the device, the second material comprises a second biodegradable material. In yet another embodiment of the device, first material is distributed in the second material. In yet another embodiment of the device, the first material is configured to create pores in the second material. In yet another embodiment of the device, the pores allow the drug to flow from within the bead to outside the bead. In yet another embodiment of the device, the metal-containing material is configured to form pores in the shell, before or after implanting or attaching the device in or on the body of the subject, when the bead is exposed to an external stimulus. In yet another embodiment of the device, the metal-containing material comprises metallic particles. In yet another embodiment of the device, the metallic particles comprise at least one of an iron-containing particles a manganese-containing particle, an iron-containing polymer, and a manganese-containing polymer. In yet another embodiment of the device, the first and second materials comprise polymers. In yet another embodiment of the device, the first material comprises polylactic acid (PLA) or an iron-containing polymer and the second material comprises poly E-caprolactone (PCL). In yet another embodiment of the device, the bead further contains a polymer. In yet another embodiment of the device, the desired flow rate of elution of the drug is substantially constant. In yet another embodiment of the device, a desired flow rate of elution of the drug is substantially constant for a first period of time and substantially zero for a second period of time, or vice versa. In yet another embodiment of the device, a desired flow rate of elution of the drug is increasing or decreasing with time. In yet another embodiment of the device, the electronics unit further comprises a chip comprising a cyber security module. In yet another embodiment of the device, the device is disposable. In yet another embodiment of the device, the device is reusable. In yet another embodiment of the device, the device further comprises a communication element for communicating with remote devices. In yet another embodiment of the device, the communication element is a magnetic induction device. In yet another embodiment of the device, the communication element is an infra-red device. In yet another embodiment of the device, the communication element is a radiofrequency device. In yet another embodiment of the device, the drug reservoir unit is refillable. In yet another embodiment of the device, for every firing of the electromagnetic actuator, the displacement unit delivers the constant volume of drug via the drug delivery unit by moving a constant distance in only one direction.

Another embodiment relates to a device comprising: a first chamber comprising a drug reservoir unit and an osmotic unit; a second chamber comprising an electronic unit configured to provide an electromagnetic force to the displacement unit; and a third chamber comprising a drug delivery unit; a displacement unit comprising an electromagnetic actuator and a micro-holed plate for fluid flow, the electromagnetic actuator comprising a piston, and a spring, and wherein the device is configured to deliver a drug into body of a subject; and wherein, when the device is implanted in the body of the subject, a first maximum force exerted on the micro-holed plate for the fluid flow therein by a hydrostatic pressure within the drug reservoir unit is F1, a second maximum force exerted on the micro-holed plate by the spring is F2, and a maximum force exerted by the electromagnetic force is F3, wherein F1 is less than F2 and F2 is less than F3.

Another embodiment relates to a device comprising: a first chamber comprising a drug reservoir unit and an osmotic unit comprising a semipermeable membrane plate; a second chamber comprising an electronic unit; a third chamber comprising a drug delivery unit; a displacement unit comprising an electromagnetic actuator, the electromagnetic actuator comprising a piston a solenoid and a spring; and wherein the device is configured to deliver a drug into body of a subject; wherein the device is implantable into the body of the body of the subject; and wherein the semipermeable membrane plate is non retractable and moves only in one direction.

Another embodiment relates to a device comprising: an osmotic unit comprising a semipermeable membrane plate; a second chamber comprising an electronic unit; a third chamber comprising a drug delivery unit; a displacement unit comprising an electromagnetic actuator, the electromagnetic actuator comprising a piston a solenoid and a spring; and wherein the device is configured to deliver a drug into body of a subject; wherein the device is implantable into the body of the body of the subject; and wherein the piston is non retractable and moves only in one direction.

An embodiment relates to a device comprising a first chamber comprising an osmotic unit; a second chamber comprising a drug suspension, a third chamber comprising a drug delivery unit, and an ON/OFF flow switch, and a fourth chamber comprising an electronic unit comprising a control component; wherein the flow of drug via the ON/OFF switch is controlled via the electronic unit; wherein the device is fully implanted in a subject during operation, and wherein the implantable device releases a drug into the subject at a flow rate such that a variation in the flow rate is within ±15% by volume.

An embodiment relates a system, comprising: a device comprising a first chamber comprising an osmotic unit, a second chamber comprising a drug suspension, a third chamber comprising a drug delivery unit and an ON/OFF flow switch, a fourth chamber comprising an electronic unit comprising a control component, and one or more sensors configured to monitor a bioactivity, and a software-implemented module configured to determine a drug dose; wherein the flow of drug via the ON/OFF switch is controlled via the electronic unit.

An embodiment relates to a device comprising a back-end cap comprising a forward osmosis permeability membrane, a pressure sensor, and a conductivity sensor. An embodiment relates to a method of fabrication and integration of an integrated implantable device comprising an osmotic pump, a piston displacement measurement mechanism, and a suite of electromechanical actuator. An embodiment relates to a device comprising a front-end cap; the front-end comprising a ball relief valve and one of a stepper motor with helical shaft and a vibration motor, a battery and a control circuit electronics. An embodiment relates to a device comprising a tube comprising a piston comprising a back-end cap, a front-end cap, a battery, and a control circuit electronics wherein the device is operable to release a consistent amount of a liquid stored in a chamber of the tube. An embodiment relates to a device comprising a tube comprising a piston comprising a back-end cap, a front-end cap, a battery and a control circuit electronics wherein the device is operable to release a consistent amount of a liquid stored in a chamber of the tube, and wherein dosing accuracy of the device is within ±15% of a target dose. An embodiment relates to a method of manufacturing a device by comprising a cylindrical tube comprising a piston with a back-end cap and a front-end cap.

### BRIEF DESCRIPTION OF THE FIGURES

These and other aspects of the present disclosure will now be described in more detail, with reference to the appended drawings showing exemplary embodiments of the present disclosure, in which:

FIG. 1 shows a schematic of an implantable device according to one embodiment disclosed herein.

FIG. 2 (prior art) is a diagram showing how DUROS' implantable device works.

FIG. 3 shows a schematic of a substantially constant drug concentration profile with time according to one embodiment disclosed herein.

FIG. 4 shows a schematic of a plate containing holes, some, or all of which are filled with phase-change material (PCM).

FIG. 5 shows a schematic of a foam plug containing the plate of FIG. 4, further showing electrode 1 and electrode 2 across the foam plug.

FIG. 6 (prior art) is a schematic showing how MicroCHIPS® wireless implantable device works.

FIG. 7A shows an exterior view of the implantable device, according to one or more embodiments.

FIG. 7B shows the interior of the implantable device, according to one or more embodiments.

FIG. 7C shows an exploded view of the interior of the implantable device, according to one or more embodiments.

FIG. 7D shows sub-assemblies of the implantable device, according to one or more embodiments.

FIG. 7E shows a consistent amount of drug delivery per shot by the implantable device, according to one or more embodiments.

FIG. 7F shows movement of a piston driven by osmotic pressure, according to one or more embodiments.

FIG. 7G shows a schematic of fluid flow in the implantable device, according to one or more embodiments.

FIG. 7H shows a schematic of fluid flow via a valve in the implantable device, according to one or more embodiments.

FIG. 7I shows a solenoid valve with non-revered inlet port and outlet ports and FIG. 7J. shows a solenoid valve with reversed inlet port and outlet ports.

FIG. 8A illustrates a transparent side view of the implantable device where subassemblies of the implantable device are visible, according to one or more embodiments.

FIG. 8B illustrates example forces acting in the implantable device to deliver a consistent volume of drug per shot, according to one or more embodiments.

FIG. 8C illustrates a close view of the drug delivery chamber of the implantable device, according to one or more embodiments.

FIG. 8D and FIG. 8E illustrate a close view of the displacement unit of the implantable device, according to one or more embodiments.

FIG. 8F, FIG. 8G and FIG. 8H illustrate a close view of the example components of the electronic chamber of the implantable device, according to one or more embodiments.

FIG. 8I illustrates a process pumping action by the powered piston of the implantable device, according to one or more embodiments.

FIG. 8J illustrates forces acting in the implantable device, according to one or more embodiments.

FIG. 8K illustrates sample spring embodiments for the powered piston of the implantable device.

FIG. 8L illustrates an umbrella piston of the implantable device, according to one or more embodiments.

FIG. 8M illustrates sample valve embodiments for the powered piston of the implantable device.

FIG. 9A shows a schematic of an osmotic pump based implantable device comprising a positive displacement pump, according to one or more embodiments.

FIG. 9B shows a schematic of an implantable device comprising a positive displacement pump, according to one or more embodiments.

FIG. 9C is a cross-sectional side view of the micro-pump with the pump element in a non-pumping, liquid conducting position.

FIG. 9D illustrate how the voltage source and multiplexer of the switching circuit cooperate to generate a peristaltic deformation along the longitudinal axis of the pump element in order to pump fluid disposed in the pump body.

FIG. 9E is a cross-sectional side view of the micro-pump with the pump element in a non-pumping, liquid conducting position.

FIG. 9F illustrate how the voltage source and multiplexer of the switching circuit cooperate to generate a peristaltic deformation along the longitudinal axis of the pump element in order to pump fluid disposed in the pump body.

FIG. 9G is a perspective view of the control assembly of the micropump.

FIG. 9H shows a schematic diagram of a two-chamber pump according to one embodiment of the present invention.

FIG. 9I shows inlet, outlet and total flow rates for the pump with triangular actuation.

FIG. 9J shows a schematic diagram of a multi chamber pump according to an embodiment of the present invention having chambers operating in parallel.

FIG. 9K shows a schematic diagram of a multi chamber pump according to a further embodiment of the present invention having chambers operating in series.

FIG. 9L illustrate a micro diaphragm pump according to an embodiment of the present invention.

FIG. 9M illustrate a micro diaphragm pump according to an embodiment of the present invention.

FIG. 9N is a perspective view of a section of one embodiment of the present invention embodied in a double superimposed chamber valveless MEMS micropump.

FIG. 9O is a schematic showing the operation of the micropump according to an embodiment of the current invention.

FIG. 9P shows a pressure accumulator in a cutaway of piston, according to one or more embodiments.

FIG. 9Q shows a pressure accumulator as disk in osmotic unit, according to one or more embodiments.

FIG. 10A is a graphical representation of osmotic pressure exerted by various solutes at various concentrations.

FIG. 10B is a graphical representation of osmotic pressure exerted by sodium chloride salt at four concentrations at various temperatures.

FIG. 10C shows movement of a piston driven by osmotic pressure, according to one or more embodiments.

FIG. 10D shows a schematic of a substantially constant pressure profile with time according to one embodiment disclosed herein.

FIG. 10E is a schematic showing a chip comprising a solute reservoir assembly, according to one or more embodiments.

FIG. 10F illustrates sample seal embodiments for the powered piston of the implantable device.

FIG. 11A illustrates an electronic unit of an implantable device, according to one or more embodiments.

FIG. 11B illustrates an electronic unit of an implantable device, according to one or more embodiments.

FIG. 11C illustrates an electronic unit of an implantable device, according to one or more embodiments.

FIG. 11D illustrates an electronic control unit of an implantable device, according to one or more embodiments.

FIG. 12A illustrates a power switch of an implantable device, according to one or more embodiments.

FIG. 12B shows a schematic of the electronics to control the solenoid, according to one or more embodiments.

FIG. 12C illustrates automatic sleep mode flow of the processor of the implantable device when device is not active.

FIG. 12D illustrates the broad level program flow programmed within the processor of the device.

FIG. 13A shows an embodiment of the electronics and the driver circuit to control the solenoid.

FIG. 13B shows an embodiment of the electronics and the driver circuit to control the solenoid.

FIG. 13C shows an embodiment of a simplistic representation of the driver circuit to control the electrostrictive micro-pump.

FIG. 13D shows the embodiment of a simplistic representation of the control circuit for Micro Pump for microfluidic channel.

FIG. 13E shows the embodiment of a simplistic representation of the control circuit for Micro Diaphragm Pump.

FIG. 14A is a schematic view of an embodiment of the measuring device, according to one or more embodiments, and FIG. 14B illustrates an alternative embodiment of the device of the figure.

FIG. 14C is a longitudinal sectional view schematically showing the configuration of a distance measuring apparatus according to one or more embodiments.

FIG. 14D is a sectional view through a measuring head and a reflecting object and two auxiliary reflectors, to explain the method and the device and FIG. 14E is a pulse diagram for the course of a measurement.

FIG. 14F is a view showing the overall configuration of a high accuracy displacement measuring system according to the present invention, including a laser interferometer displacement measuring system and a driving system.

FIG. 14G is a view showing the flow of signals in an exemplary configuration of distortion error corrector means according to the present invention.

FIG. 14H is a depiction of an example Time of Flight (TOF) system according to an embodiment.

FIG. 14I, FIG. 14J, FIG. 14K, and FIG. 14L are examples of the TOF system in the implantable pump according to an embodiment.

FIG. 14M is a block diagram showing an embodiment of the distance measuring device of the present invention using sound waves.

FIG. 14N is a front sectional view showing a configuration of a distance measuring device (laser distance measuring device) according to an embodiment.

FIG. 14O is a perspective view showing the configuration of the distance measuring device.

FIG. 14P shows piston position measurement based on resistance, according to one or more embodiments.

FIG. 14Q shows current flow schematic for measurement of resistance according to one or more embodiments.

FIG. 14R shows various sensors for detecting the movement of an object according to an embodiment.

FIG. 15A and FIG. 15B depict an example implant delivery device for delivering implantable device according to an embodiment of the present disclosure.

FIG. 16A shows force in an implantable device to deliver a consistent volume of drug per shot, according to one or more embodiments.

FIG. 16B shows a transparent side view of the implantable device where subassemblies of the implantable device are visible, according to one or more embodiments.

FIG. 17A shows a front view of an implantable device, according to one or more embodiments.

FIG. 17B shows a perspective view of an implantable device, according to an embodiment.

FIG. 17C shows a cross-sectional view of an implantable device, according to an embodiment.

FIG. 17D shows a first perspective view of a permeable membrane plug of an implantable device, according to an embodiment.

FIG. 17E shows a second perspective view of a permeable membrane plug of an implantable device, according to an embodiment.

FIG. 17F shows a transparent side view of a permeable membrane plug of an implantable device, according to an embodiment.

FIG. 17G shows a top view of a 3D model for a drug reservoir chamber of an implantable device, according to an embodiment.

FIG. 17H shows a transparent side-view for the drug reservoir chamber according to an embodiment.

FIG. 17I shows a perspective view of a displacement unit of an implantable device, according to an embodiment.

FIG. 17J shows a transparent side view of a displacement unit of an implantable device, according to an embodiment.

FIG. 17K shows a front view of a micro holed plate of an implantable device, according to an embodiment.

FIG. 17L shows a transparent side view of an electronic chamber of an implantable device, according to an embodiment.

FIG. 17M shows a front view of an electronic chamber of an implantable device, according to an embodiment.

FIG. 17N shows a transparent side view of an electronic chamber of an implantable device, according to an embodiment.

FIG. 18A shows a cross-sectional view of a pump according to an embodiment.

FIG. 18B shows a cross-sectional view of a pump according to an embodiment.

FIG. 18C shows a front view of the implantable device, according to an embodiment.

FIG. 18D shows a transparent side view of an implantable device, according to an embodiment.

FIG. 18E shows a front view of an electronic chamber of an implantable device, according to an embodiment.

FIG. 18F shows a transparent side view of an electronic chamber of an implantable device, according to an embodiment.

FIG. 18G shows a cross-sectional view of an implantable device, according to an embodiment.

FIG. 18H shows a cross-sectional view of an implantable device, according to an embodiment.

FIG. 18I shows a cross-sectional view of an implantable device, according to an embodiment.

FIG. 19A shows a schematic of an implantable drug delivery device, according to one or more embodiments.

FIG. 19B shows a first end cap, FIG. 19C shows a 4 mm diameter tube with a Teflon piston, and

FIG. 19D shows a front-end cap of the device, according to one or more embodiments.

FIG. 19E shows schematic of a tube with a piston, according to one or more embodiments.

FIG. 19F shows a front-end Cap assembly depicting mechanical mechanism to control of a valve, according to one or more embodiments.

FIG. 19G shows a cross section through a micropump based ON/OFF switch, according to one or more embodiments.

FIG. 19H shows a top view of a fluidic substrate of the micropump, according to one or more embodiments.

FIG. 19I and FIG. 19J illustrate a micro-valve structure and an actuation method thereof, according to one or more embodiments.

FIG. 19K, FIG. 19L, and FIG. 19M show the arrangement of a plurality of diaphragms inside the ON/OFF flow switch, according to one or more embodiments.

FIG. 19N shows various illustrations of NEXIPAL® actuator arrangements to be used in the ON/OFF flow switch in ON state and in OFF state, according to one or more embodiments.

FIG. 19O shows the device comprising an electromagnetic switch, according to one or more embodiments.

FIG. 19P shows the electromagnetic switch in ON state and FIG. 19Q shows the electromagnetic switch 612a in OFF state.

FIG. 19R shows examples of piezo elements that can be used in the flow switch of the device for ON/OFF actuation, according to one or more embodiments.

FIG. 19S shows ON/OFF mechanism for a flow switch comprising a piezoelectric stack.

FIG. 19T shows ON/OFF mechanism for a flow switch comprising a piezoelectric bender.

FIG. 19U, FIG. 19V, FIG. 19W, FIG. 19X and FIG. 19Y illustrate the loading and release procedure of a straight Shape Memory Polymer (SMP) hollow member or tubing being press-fitted over the ball-ended coil, according to one or more embodiments.

FIG. 19Z shows ON/OFF mechanism for a flow switch comprising a Shape Memory Alloy (an SMA) actuator.

FIG. 19AA and FIG. 19AB show ON/OFF mechanism for a flow switch comprising an electrostatic actuator, according to one or more embodiments.

FIG. 19AC shows an example of a flow switch comprising a rotatory actuation mechanism.

FIG. 19AD illustrates an example of a flow switch utilizing a gear-driven toothed gate mechanism.

FIG. 19AE depicts an example of a flow switch employing a screw-driven actuation mechanism housed within a housing chamber.

FIG. 19AF shows an example of a flow switch comprising a thermopneumatic actuation mechanism.

FIG. 19AG illustrates a device comprising resistive stripes and a piston position monitoring module, according to one or more embodiments.

FIG. 19AH illustrates a device comprising an LED reflection, measured by a photodiode, converting reflected light into an electrical signal for piston position monitoring, according to one or more embodiments.

FIG. 19AI illustrates a device comprising an inner circumference of the drug suspension chamber, comprising a pattern of equally spaced plurality of rings in which the piston shorts a circuit when coming into contact with a set of the rings to communicate the piston position (drug dispensed), according to one or more embodiments.

FIG. 19AJ shows the relationship between conductivity G and salt concentration C.

FIG. 19AK illustrates a device comprising electrodes for measurement of salt conductance and a corresponding circuit diagram, according to one or more embodiments.

FIG. 19AL shows a circuit diagram to measure conductance of the salt solution, via the device, according to one or more embodiments.

FIG. 19AM shows a drug delivery device comprising an osmotic pressure-based piston displacement monitoring module, according to one or more embodiments.

FIG. 19AN shows an exploded view of the end cap of the device tube, which comprises a semi-permeable membrane and an osmotic pressure-based piston displacement monitoring module.

FIG. 19AO shows a drug delivery device comprising a pressure sensor and a piston position monitoring module, according to one or more embodiments.

FIG. 19AP and FIG. 19AQ depict the simulation and experimental results obtained from the device incorporating a conductivity sensor.

FIG. 19AR shows a drug delivery device comprising a resistive touch membrane lining circuit diagram and a piston position monitoring module, according to one or more embodiments.

FIG. 19AS and FIG. 19AT show a schematic of an implantable drug delivery device comprising a relief valve in OFF state and ON state, according to one or more embodiments.

FIG. 19AU shows a schematic of ON/OFF mechanism of flow via relief valve, according to one or more embodiments.

FIG. 19AV shows schematic of ON/OFF mechanism of a super stroke solenoid for controlled drug release, according to one or more embodiments.

FIG. 19AW shows a device comprising a check valve and a stepper motor actuator in an ON state, according to one or more embodiments.

FIG. 19AX shows an implantable device comprising a check valve and vibration motor actuator in an ON state, according to one or more embodiments.

FIG. 19AY and FIG. 19AZ show a schematic of an implantable drug delivery device comprising a spring-loaded check valve in OFF state and ON state, according to one or more embodiments.

FIG. 19BA shows a schematic of ON/OFF mechanism of flow via the spring-loaded check valve, according to one or more embodiments.

FIG. 19BB is an axial sectional view of a check valve in accordance with the present invention illustrated in combination with a fluid conduit in which the valve has been mounted.

FIG. 19BC is a fragmentary side view of a selected portion of the check valve of FIG. 19BB; and

FIG. 19BD is a fragmentary cross-sectional view of the check valve and fluid conduit taken along the line 19bc03-19bc03 of FIG. 19BC.

FIG. 19BE shows a proof-of-concept demonstration of a device, according to one or more embodiments.

FIG. 19BF illustrates a valve assembly designed to regulate fluid flow in the device, according to one or more embodiments.

FIG. 19BG illustrates an implantable device for single dose delivery, according to one or more embodiments.

FIG. 19BH illustrates an implantable device comprising a pressurized first chamber comprising water and CO₂, according to one or more embodiments.

FIG. 19BI illustrates an implantable device comprising a pressurized first chamber comprising a compressed spring, according to one or more embodiments.

FIG. 20A shows the Shape-Memory Alloy (SMA) based valve according to an embodiment, where the valve is in closed position.

FIG. 20B shows the SMA based valve according to an embodiment, where the valve is in open position due to SMA element being activated.

FIG. 20C shows the SMA based valve according to an embodiment, where the valve is in closed position.

FIG. 20D shows the SMA based valve according to an embodiment, where the valve is in open position due to SMA element being activated.

FIG. 20E and FIG. 20F depict a diagrammatic side view of an example, non-limiting embodiment of a piezoelectric valve.

FIG. 20G illustrates an exemplary configuration for actuating the diaphragm valve.

FIG. 20H illustrates the diaphragm valve of FIG. 20G actuated to an open position.

FIG. 20I and FIG. 20J show electromagnetic microvalve integrated on the microfluidic chip, according to one or more embodiments.

FIG. 20K shows the energization of electromagnet under the action of the magnetic field.

FIG. 20L shows fluid passage at a completely open state of electromagnetic valve.

FIG. 20M shows a component of solenoid valve according to one or more embodiments.

FIG. 20N is a front diagram showing an outline of a driving mechanism in an extended state, the driving mechanism using a shape memory alloy wire that drives each of a micropump and a microvalve in an embodiment of the present disclosure.

FIG. 20O is a front diagram showing an outline of the driving mechanism in a contracted state, the driving mechanism using the shape memory alloy wire that drives each of the micropump and the microvalve in an embodiment of the present disclosure.

FIG. 20P and FIG. 20Q are diagrams showing an electrostatic microvalve according to one embodiment.

FIG. 20R shows a state in which no voltage is applied to the electrodes of the pressurized tank and a sample can flow through the sample flow path.

FIG. 20S shows a state in which a voltage is applied from the power supply device.

FIG. 20T is a sectioned side view of another example embodiment of a device comprising a pump comprising active valves and a timer for controlling duration of valve operation.

FIG. 20U is a sectioned side view of another example embodiment of the device comprising active valves and a timer for controlling duration of valve operation.

FIG. 20V is a sectioned side view of another example embodiment of the device comprising active valves and a timer for controlling duration of valve operation with outlet valve perpendicular to inlet valve.

FIG. 20W is a schematic structural view of a preferred embodiment of the single side opening and closing of a single fluid microchannel of the phase change microvalve device of the present invention.

FIG. 20X is a schematic diagram of a preferred structure of the double-sided opening and closing of a single micro fluid channel of the phase change microvalve device of the present invention; and

FIG. 20Y is a schematic structural view of an embodiment of the simultaneous on/off regulation of a multi-fluid micro-channel of the phase change micro-valve device of the present invention.

FIG. 20Z shows embodiments of the invention of an ejector operating according to a valve principle.

FIG. 20AA shows an embodiment according to the invention operating as a fluid displacement ejector.

FIG. 20BB, A-C provide a schematic diagram illustrating various stages of a micro-fluidic channeling device according to an embodiment of the present invention.

FIG. 21A depicts various possible scenarios where an implantable device communicates with the outside world.

FIG. 21B shows communication between an implantable device and an external device using backscattering, according to one or more embodiments.

FIG. 21C depicts a simplistic implementation of a mesh network of multiple implanted devices where the implanted devices can communicate among themselves without needing the external network connection.

FIG. 21D illustrates implanted device communicating with a Smartphone via Bluetooth/RF communication.

FIG. 21E is an illustration of a system where a specially designed device acts as an information relay between the implanted device and remote monitoring clinic system.

FIG. 21F is a simplistic block diagram of the Smart Link Device Hardware Architecture.

FIG. 21G illustrates the high-level architecture of the Mobile Application provided on the patient's smartphone.

FIG. 22A depicts (a) an arrangement for power supply to the implantable device through inductive coupling and (b) a functional block diagram of an example of a wireless power transfer system.

FIG. 22B shows a diagram illustrating an example embodiment of a biofuel cell powered electronic circuit in accordance with the present technology.

FIG. 23A illustrates an exterior view of a multiple drug delivery implantable device, according to an embodiment.

FIG. 23B illustrates a multi-drug multi-valve device, according to one or more embodiments.

FIG. 24A shows a schematic of electronic components of the implantable device, according to one or more embodiments.

FIG. 24B shows a schematic of control module of the implantable device, according to one or more embodiments.

FIG. 24C is a diagram of a piezoelectric actuator stack in accordance with one embodiment.

FIG. 25A illustrates a representative implant system that comprises tapped coil antenna.

FIG. 25B shows schematic of an implant adapted to receive wireless power from an external transceiver via an ultrasound signal.

FIG. 25C depicts a galvanic Electro-Quasistatic—Human Body Communication system utilizing body tissue as the medium of communication.

FIG. 25D shows a diagram illustrating an example embodiment of a biofuel cell powered electronic circuit in accordance with the present technology.

FIG. 26A shows an implantable device comprising biosensors for bioactivity measurement, according to one or more embodiments.

FIG. 26B shows a system comprising biosensors for bioactivity measurement and personalized drug dose delivery, according to one or more embodiments.

FIG. 27 shows a system for drug precision dosing of a drug, according to one or more embodiments.

FIG. 28A shows a structure of the neural network/machine learning model with feedback loop.

FIG. 28B shows a structure of the neural network/machine learning model with reinforcement learning.

FIG. 28C shows an example block diagram for detecting one or more prognosticators, indicators, and risk factors of postoperative performance of the implanted device using a machine learning model, according to one or more embodiments.

FIG. 28D shows a data flow in a system comprising an implantable device according to one or more embodiments.

FIG. 28E is a block diagram of an example device that may be used externally of a patient's body, and that may communicate with an implantable medical device, in accordance with one or more implementations.

FIG. 29A shows an example flow chart for detecting device anomalies using a machine learning model, according to one or more embodiments.

FIG. 29B illustrates the AI architecture for pre-implantation training of the implantable device, following one or more embodiments.

FIG. 29C illustrates the AI architecture for post-implantation training of the implantable device, following one or more embodiments.

FIG. 29D illustrates the AI Architecture for implantable monitoring, according to one or more embodiments.

FIG. 30 shows an example flow chart of a calibration process according to some embodiments or methods of the present disclosure.

FIG. 31 shows a block diagram of the cyber security module in view of the system and server.

FIG. 32A shows an embodiment of the cyber security module.

FIG. 32B shows another embodiment of the cyber security module.

### DETAILED DESCRIPTION

For simplicity and clarity of illustration, the figures illustrate the general manner of construction. The description and figures may omit the descriptions and details of well-known features and techniques to avoid unnecessarily obscuring the present disclosure. The figures exaggerate the dimensions of some of the elements relative to other elements to help improve understanding of embodiments of the present disclosure. The same reference numeral in different figures denotes the same element.

Although the detailed description herein contains many specifics for the purpose of illustration, a person of ordinary skill in the art will appreciate that many variations and alterations to the details are considered to be included herein.

Accordingly, the embodiments herein are without any loss of generality to, and without imposing limitations upon, any claims set forth. The terminology used herein is for the purpose of describing particular embodiments only and is not limiting. Unless defined otherwise, all technical and scientific terms used herein have the same meaning as commonly understood by one with ordinary skill in the art to which this disclosure belongs.

As used herein, the articles “a” and “an” used herein refer to one or to more than one (i.e., to at least one) of the grammatical object of the article. By way of example, “an element” means one element or more than one element. Moreover, usage of articles “a” and “an” in the subject specification and annexed drawings construe to mean “one or more” unless specified otherwise or clear from context to mean a singular form.

As used herein, the terms “example” and/or “exemplary” mean serving as an example, instance, or illustration. For the avoidance of doubt, such examples do not limit the herein described subject matter. In addition, any aspect or design described herein as an “example” and/or “exemplary” is not necessarily preferred or advantageous over other aspects or designs, nor does it preclude equivalent exemplary structures and techniques known to those of ordinary skill in the art.

As used herein, the terms “first,” “second,” “third,” and the like in the description and in the claims, if any, distinguish between similar elements and do not necessarily describe a particular sequence or chronological order. The terms are interchangeable under appropriate circumstances such that the embodiments herein are, for example, capable of operation in sequences other than those illustrated or otherwise described herein. Furthermore, the terms “include,” “have,” and any variations thereof, cover a non-exclusive inclusion such that a process, method, system, article, device, or apparatus that comprises a list of elements is not necessarily limiting to those elements, but may include other elements not expressly listed or inherent to such process, method, system, article, device, or apparatus.

As used herein, the terms “left,” “right,” “front,” “back,” “top,” “bottom,” “over,” “under” and the like in the description and in the claims, if any, are for descriptive purposes and not necessarily for describing permanent relative positions. The terms so used are interchangeable under appropriate circumstances such that the embodiments of the apparatus, methods, and/or articles of manufacture described herein are, for example, capable of operation in other orientations than those illustrated or otherwise described herein.

No element act, or instruction used herein is critical or essential unless explicitly described as such. Furthermore, the term “set” includes items (e.g., related items, unrelated items, a combination of related items and unrelated items, etc.) and may be interchangeable with “one or more”. Where only one item is intended, the term “one” or similar language is used. Also, the terms “has,” “have,” “having,” or the like are open-ended terms. Further, the phrase “based on” means “based, at least in part, on” unless explicitly stated otherwise.

As used herein, the terms “system,” “device,” “unit,” and/or “module” refer to a different component, component portion, or component of the various levels of the order. However, other expressions that achieve the same purpose may replace the terms.

As used herein, the terms “couple,” “coupled,” “couples,” “coupling,” and the like refer to connecting two or more elements mechanically, electrically, and/or otherwise. Two or more electrical elements may be electrically coupled together, but not mechanically or otherwise coupled together. Coupling may be for any length of time, e.g., permanent, or semi-permanent or only for an instant. “Electrical coupling” includes electrical coupling of all types. The absence of the word “removably,” “removable,” and the like, near the word “coupled” and the like does not mean that the coupling, etc. in question is or is not removable.

As used herein, the term “or” means an inclusive “or” rather than an exclusive “or.” That is, unless specified otherwise, or clear from context, “X employs A or B” means any of the natural inclusive permutations. That is, if X employs A; X employs B; or X employs both A and B, then “X employs A or B” is satisfied under any of the foregoing instances.

As used herein, two or more elements or modules are “integral” or “integrated” if they operate functionally together. Two or more elements are “non-integral” if each element can operate functionally independently.

As used herein, the term “real-time” refers to operations conducted as soon as practically possible upon occurrence of a triggering event. A triggering event can include receipt of data necessary to execute a task or to otherwise process information. Because of delays inherent in transmission and/or in computing speeds, the term “real-time” encompasses operations that occur in “near” real-time or somewhat delayed from a triggering event. In a number of embodiments, “real-time” can mean real-time less a time delay for processing (e.g., determining) and/or transmitting data. The particular time delay can vary depending on the type and/or amount of the data, the processing speeds of the hardware, the transmission capability of the communication hardware, the transmission distance, etc. However, in many embodiments, the time delay can be less than approximately one second, two seconds, five seconds, or ten seconds.

As used herein, the term “approximately” can mean within a specified or unspecified range of the specified or unspecified stated value. In some embodiments, “approximately” can mean within plus or minus ten percent of the stated value. In other embodiments, “approximately” can mean within plus or minus five percent of the stated value. In further embodiments, “approximately” can mean within plus or minus three percent of the stated value. In yet other embodiments, “approximately” can mean within plus or minus one percent of the stated value.

As used herein, the term “substantially” or the word “roughly” or the word “about” is used to qualify (in particular) numerical quantities to indicate that small variations from the specified value are envisaged.

Other specific forms may embody the present disclosure without departing from its spirit or characteristics. The described embodiments are in all respects illustrative and not restrictive.

Therefore, the appended claims rather than the description herein indicate the scope of the disclosure. All variations which come within the meaning and range of equivalency of the claims are within their scope.

The actual specialized control hardware or software code used to implement these systems and/or methods is not limiting to the implementations. Thus, any software and any hardware can implement the systems and/or methods based on the description herein without reference to specific software code.

A computer program (also known as a program, software, software application, script, or code) is written in any appropriate form of programming language, including compiled or interpreted languages. Any appropriate form, including a standalone program or a module, component, subroutine, or other unit suitable for use in a computing environment may deploy it. A computer program does not necessarily correspond to a file in a file system. A program may be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program may execute on one computer or on multiple computers that are located at one site or distributed across multiple sites and interconnected by a communication network.

One or more programmable processors, executing one or more computer programs to perform functions by operating on input data and generating output, perform the processes and logic flows described in this specification. The processes and logic flows may also be performed by, and apparatus may also be implemented as, special purpose logic circuitry, for example, without limitation, a Field Programmable Gate Array (FPGA), an Application Specific Integrated Circuit (ASIC), Application Specific Standard Products (ASSPs), System-On-a-Chip (SOC) systems, Complex Programmable Logic Devices (CPLDs), etc.

Processors suitable for the execution of a computer program include, by way of example, both general and special purpose microprocessors, and any one or more processors of any appropriate kind of digital computer. A processor will receive instructions and data from a read-only memory or a random-access memory or both. Elements of a computer can include a processor for performing instructions and one or more memory devices for storing instructions and data. A computer will also include, or is operatively coupled to receive data, transfer data or both, to/from one or more mass storage devices for storing data e.g., magnetic disks, magneto optical disks, optical disks, or solid-state disks. However, a computer need not have such devices. Moreover, another device, e.g., a mobile telephone, a personal digital assistant (PDA), a mobile audio player, a Global Positioning System (GPS) receiver, etc. may embed a computer. Computer-readable media suitable for storing computer program instructions and data include all forms of non-volatile memory, media and memory devices, including, by way of example, semiconductor memory devices (e.g., Erasable Programmable Read-Only Memory (EPROM), Electronically Erasable Programmable Read-Only Memory (EEPROM), and flash memory devices), magnetic disks (e.g., internal hard disks or removable disks), magneto optical disks (e.g. Compact Disc Read-Only Memory (CD ROM) disks, Digital Versatile Disk-Read-Only Memory (DVD-ROM) disks) and solid-state disks. Special purpose logic circuitry may supplement or incorporate the processor and the memory.

To provide for interaction with a user, a computer may have a display device, e.g., a Cathode Ray Tube (CRT) or Liquid Crystal Display (LCD) monitor, for displaying information to the user, and a keyboard and a pointing device, e.g., a mouse or a trackball, by which the user may provide input to the computer. Other kinds of devices provide for interaction with a user as well. For example, feedback to the user may be any appropriate form of sensory feedback, e.g., visual feedback, auditory feedback, or tactile feedback; and a computer may receive input from the user in any appropriate form, including acoustic, speech, or tactile input.

A computing system that includes a back-end component, e.g., a data server, or that includes a middleware component, e.g., an application server, or that includes a front-end component, e.g., a client computer having a graphical user interface or a Web browser through which a user may interact with an implementation, or any appropriate combination of one or more such back-end, middleware, or front-end components, may realize implementations described herein. Any appropriate form or medium of digital data communication, e.g., a communication network may interconnect the components of the system. Examples of communication networks include a Local Area Network (LAN) and a Wide Area Network (WAN), e.g., Intranet and Internet.

The computing system may include clients and servers. A client and server are remote from each other and typically interact through a communication network. The relationship of the client and server arises by virtue of computer programs running on the respective computers and having a client-server relationship with each other.

Embodiments of the present disclosure may comprise or utilize a special purpose or general-purpose computer including computer hardware. Embodiments within the scope of the present disclosure may also include physical and other computer-readable media for carrying or storing computer-executable instructions and/or data structures. Such computer-readable media can be any media accessible by a general purpose or special purpose computer system. Computer-readable media that store computer-executable instructions are physical storage media. Computer-readable media that carry computer-executable instructions are transmission media. Thus, by way of example and not limitation, embodiments can comprise at least two distinct kinds of computer-readable media: physical computer-readable storage media and transmission computer-readable media.

Although the present embodiments described herein are with reference to specific example embodiments it will be evident that various modifications and changes may be made to these embodiments without departing from the broader spirit and scope of the various embodiments. For example, hardware circuitry (e.g., Complementary Metal Oxide Semiconductor (CMOS) based logic circuitry), firmware, software (e.g., embodied in a non-transitory machine-readable medium), or any combination of hardware, firmware, and software may enable and operate the various devices, units, and modules described herein. For example, transistors, logic gates, and electrical circuits (e.g., Application Specific Integrated Circuit (ASIC) and/or Digital Signal Processor (DSP) circuit) may embody the various electrical structures and methods.

In addition, a non-transitory machine-readable medium and/or a system may embody the various operations, processes, and methods disclosed herein. Accordingly, the specification and drawings are illustrative rather than restrictive.

Physical computer-readable storage media includes RAM, ROM, EEPROM, CD-ROM or other optical disk storage (such as CDs, DVDs, etc.), magnetic disk storage or other magnetic storage devices, solid-state disks or any other medium. They store desired program code in the form of computer-executable instructions or data structures which can be accessed by a general purpose or special purpose computer.

As used herein, the term “network” refers to one or more data links that enable the transport of electronic data between computer systems and/or modules and/or other electronic devices. When a network or another communications connection (either hardwired, wireless, or a combination of hardwired or wireless) transfers or provides information to a computer, the computer properly views the connection as a transmission medium. A general purpose or special purpose computer access transmission media that can include a network and/or data links which carry desired program code in the form of computer-executable instructions or data structures. The scope of computer-readable media includes combinations of the above, that enable the transport of electronic data between computer systems and/or modules and/or other electronic devices.

Further, upon reaching various computer system components, program code in the form of computer-executable instructions or data structures can be transferred automatically from transmission computer-readable media to physical computer-readable storage media (or vice versa). For example, computer-executable instructions or data structures received over a network or data link can be buffered in RAM within a Network Interface Module (NIC), and then eventually transferred to computer system RAM and/or to less volatile computer-readable physical storage media at a computer system. Thus, computer system components that also (or even primarily) utilize transmission media may include computer-readable physical storage media.

Computer-executable instructions comprise, for example, instructions and data which cause a general-purpose computer, special purpose computer, or special purpose processing device to perform a certain function or group of functions. The computer-executable instructions may be, for example, binary, intermediate format instructions such as assembly language, or even source code. Although the subject matter herein described is in a language specific to structural features and/or methodological acts, the described features or acts described do not limit the subject matter defined in the claims. Rather, the herein described features and acts are example forms of implementing the claims.

While this specification contains many specifics, these do not construe as limitations on the scope of the disclosure or of the claims, but as descriptions of features specific to particular implementations. A single implementation may implement certain features described in this specification in the context of separate implementations. Conversely, multiple implementations separately or in any suitable sub-combination may implement various features described herein in the context of a single implementation. Moreover, although features described herein as acting in certain combinations and even initially claimed as such, one or more features from a claimed combination may in some cases be excised from the combination, and the claimed combination may be directed to a sub-combination or variation of a sub-combination.

Similarly, while operations depicted herein in the drawings in a particular order to achieve desired results, this should not be understood as requiring that such operations be performed in the particular order shown or in sequential order or that all illustrated operations be performed, to achieve desirable results. In certain circumstances, multitasking and parallel processing may be advantageous. Moreover, the separation of various system components in the implementations should not be understood as requiring such separation in all implementations, and it should be understood that the described program components and systems may be integrated together in a single software product or packaged into multiple software products.

Even though particular combinations of features are recited in the claims and/or disclosed in the specification, these combinations are not intended to limit the disclosure of possible implementations. Other implementations are within the scope of the claims. For example, the actions recited in the claims may be performed in a different order and still achieve desirable results. In fact, many of these features may be combined in ways not specifically recited in the claims and/or disclosed in the specification. Although each dependent claim may directly depend on only one claim, the disclosure of possible implementations includes each dependent claim in combination with every other claim in the claim set.

Further, a computer system including one or more processors and computer-readable media such as computer memory may practice the methods. In particular, one or more processors execute computer-executable instructions, stored in the computer memory, to perform various functions such as the acts recited in the embodiments.

Those skilled in the art will appreciate that the invention may be practiced in network computing environments with many types of computer system configurations including personal computers, desktop computers, laptop computers, message processors, hand-held devices, multi-processor systems, microprocessor-based or programmable consumer electronics, network PCs, minicomputers, mainframe computers, mobile telephones, PDAs, pagers, routers, switches, etc. Distributed system environments where local and remote computer systems, which are linked (either by hardwired data links, wireless data links, or by a combination of hardwired and wireless data links) through a network, both perform tasks may also practice the invention. In a distributed system environment, program modules may be located in both local and remote memory storage devices.

### A. Definitions

The following terms and phrases, unless otherwise indicated, shall be understood to have the following meanings:

As used herein, a “Sensor” is a device that measures physical input from its environment and converts it into data that is interpretable by either a human or a machine. Most sensors are electronic, which presents electronic data, but some are simpler, such as a glass thermometer, which presents visual data.

As used herein, “Primary sensors” are the sensors attached to the implantable device.

As used herein, “Secondary sensors” are the sensors not attached to the implantable device but used by the system predictions and recommendation.

Present disclosure provides a device for controlled delivery of drugs.

“Drug” in context of the present disclosure may include any therapeutic active agent and/or a biologically active agent (i.e., an active ingredient in a pharmaceutical composition that is biologically active, such as a vaccine), irrespective of the molecular weight of such agents. The terms “drug”, “active agent”, “therapeutic agent”, “beneficial agent” or “pharmaceutical fluid” are used interchangeably. The term “drug” or “pharmaceutical fluid” as used herein refers to a single drug or multiple types of drugs.

The phrase “single-phase” as used herein refers to a solid, semisolid, or liquid homogeneous system that is physically and chemically uniform throughout.

The term “dispersed” as used herein refers to dissolving, dispersing, suspending, or otherwise distributing a compound, for example, a peptide, in a suspension vehicle.

A “homogeneous suspension” typically refers to a particle that is insoluble in a suspension vehicle and is distributed uniformly in a suspension vehicle.

The term “substantially zero-order kinetics” means that over a medically acceptable percentage of the dose of a therapeutic agent provided in a drug delivery device, the rate of release of the agent is approximately constant such that the variability in flow is within plus or minus 15% by volume flow.

The term “pseudo zero order flux” means to maintain the rate of release of a beneficial agent almost constant such that the variability in flow is within plus minus 15% by volume flow of the solvent across the semipermeable membrane inside the osmotic pump of the implantable device.

In one embodiment, the present disclosure provides a device for controlled delivery of drugs, comprising micro or nano beads that contains a drug, wherein the beads are embedded in a polymer.

“Micro or nano beads” in context of the present disclosure have an ability to release a drug in a controlled manner. The beads may be spherical or substantially spherical in shape, having their largest transverse dimension equivalent to the diameter of the bead. Alternatively, the bead may be non-spherical, for example, ellipsoidal or tetrahedral in shape having its largest transverse dimension equivalent to the greatest distance within the bead from one bead surface to another, e.g., the major axis length for an ellipsoidal bead or the length of the longest side for a tetrahedral bead.

The term “Chamber” or “compartment” as used herein refers to a closed space or compartmentalized space within a device.

The term “Unit” as used herein may refer to a single thing/component or a group of things/components acting as one (unit) and part of something larger. A chamber may contain more than one unit. A unit may contain more than one component. According to an embodiment, the unit may comprise multiple components designed to move in unison. For example, multiple components of a displacement unit may move together under solenoid force as a single unit. In an embodiment, a chamber is a fixed length enclosure while a unit can have a variable structure. In an embodiment, the unit is a movable structure. In another embodiment, the unit is an expandable structure. In another embodiment, it is a compressible structure.

The term “fit” as used herein refers to whether the implantable device is correctly placed inside the body of the subject, or it is improperly placed and/or penetrating into a tissue. In an embodiment, a fit may include prediction of suitability of the implantable device.

The term “Sonicator” refers to a device that is used to apply sound energy to agitate particles in a sample. It also refers to a device for treatment with ultrasound.

The term “Insonated fluid” refers to a fluid exposed to or treated with ultrasound.

The term, “Penetration leakage” means leakage or flow of fluid through the seal in the width direction of a seal.

The term, “Dimples” are dents having opening portions surrounded by the flat sliding face S (land portion) and having bottom portions recessed more than the sliding face S, and the shape of the dimples is not particularly limited. For example, the shape of the opening portions of the dents includes a circle, a triangle, an ellipse, an oval, or a rectangle. The sectional shape of the dents also includes various shapes such as a cone, a truncated cone, a semi-circle, a bowl shape, or a square. The dimples are arranged so as not to overlie each other.

As used herein “Machine learning” refers to algorithms that give a computer the ability to learn without explicit programming, including algorithms that learn from and make predictions about data. Machine learning algorithms include, but are not limited to, decision tree learning, artificial neural networks (ANN) (also referred to herein as a “neural net”), deep learning neural network, support vector machines, rules-based machine learning, random forest, etc. For the purposes of clarity, part of a machine learning process can use algorithms such as linear regression or logistic regression. However, using linear regression or another algorithm as part of a machine learning process is distinct from performing a statistical analysis such as regression with a spreadsheet program. The machine learning process can continually learn and adjust the classifier as new data becomes available and does not rely on explicit or rules-based programming. The ANN may be featured with a feedback loop to adjust the system output dynamically as it learns from the new data as it becomes available. In machine learning, backpropagation and feedback loops are used to train the AI/ML model improving the model's accuracy and performance over time.

Statistical modeling relies on finding relationships between variables (e.g., mathematical equations) to predict an outcome.

As used herein, the term “Data mining” is a process used to turn raw data into useful information.

As used herein, the term “Data set” (or “Dataset”) is a collection of data. In the case of tabular data, a data set corresponds to one or more database tables, where every column of a table represents a particular variable, and each row corresponds to a given record of the data set in question. The data set lists values for each of the variables, such as height and weight of an object, for each member of the data set. Each value is known as a datum. Data sets can also consist of a collection of documents or files.

“Foreign body response” as used herein, refers to the immunological response of biological tissue to the presence of any foreign material in the tissue which can include protein adsorption, infiltration by immune cells or fibrosis.

An embodiment relates to implantable device systems for controlled delivery of drugs, especially for managing therapies for chronic patients. Therapy delivery to treat chronic ailments requires multiple administrations of a single or a multiple drug cocktail over a long period of time. Sustained delivery is highly desirable for delivery of bioactive agents particularly biologicals like peptides, antibodies, and nucleic acid analogs. This kind of delivery would provide optimum therapeutic efficacy with minimum side effects and thereby improve patient compliance.

Preferably, the implantable device therapeutic systems are built from bio-absorbable material. These therapeutic systems deliver the drug to an in vivo patient site and can occupy that site for extended periods of time without being harmful to the host.

The device of the present disclosure is advantageous over transdermal patches. The transdermal drug delivery system has several limitations since skin forms a very effective barrier and thus the system is only suitable for medications that have a small enough size to penetrate the skin, such as molecules having molecular weight less than 500. Further, molecules with sufficient aqueous and lipid solubility, having an octanol/water partition coefficient (log P) between 1 and 3 is required to permeate to transverse subcutaneous and underlying aqueous layers. Patches are known to have side effects like erythema, itching, local edema; and an allergic reaction can be caused by the drug, the adhesive, or other excipients in the patch formulation. Also, dose dumping is one of the serious implications of a patch. In one embodiment, the device of the present disclosure overcomes these limitations of a transdermal patch.

### B. Suitable Drugs

In one embodiment, the present disclosure relates to a device for controlled delivery of drugs, wherein the device is suitable for delivery of lidocaine, diclofenec, clonidine, estradiol, estradiol/norethindrone acetate, estradiol/levonorgestrel, fentanyl, methylphenidate, nicotine, norelgestromin/ethinyl estradiol, nitroglycerin, oxybutynin, scopolamine, selegiline, testosterone, rivastigmine, rotogotine.

### 1. Analgesic Agent

In an embodiment, drug is an “analgesic agent”. It refers to an agent or compound that can reduce, relieve, or eliminate pain. Examples of analgesic agents include but are not limited to acetaminophen, a local anesthetic, such as for example, lidocaine, bupivicaine, ropivacaine, opioid analgesics such as buprenorphine, butorphanol, dextromoramide, dezocine, dextropropoxyphene, diamorphine, fentanyl, alfentanil, sufentanil, hydrocodone, hydromorphone, ketobemidone, levomethadyl, levorphanol, mepiridine, methadone, morphine, nalbuphine, opium, oxycodone, papaveretum, pentazocine, pethidine, phenoperidine, piritramide, dextropropoxyphene, remifentanil, sufentanil, tilidine, tramadol, codeine, dihydrocodeine, meptazinol, dezocine, eptazocine, flupirtine or a combination thereof.

### 2. Anti-Inflammatory Agent

In an embodiment, drug is an “anti-inflammatory agent” refers to an agent or compound that has anti-inflammatory effects. These agents may remedy pain by reducing inflammation. Examples of anti-inflammatory agents include, but are not limited to, a statin, sulindac, sulfasalazine, naroxyn, diclofenac, indomethacin, ibuprofen, flurbiprofen, ketoprofen, aclofenac, aloxiprin, aproxen, aspirin, diflunisal, fenoprofen, mefenamic acid, naproxen, phenylbutazone, piroxicam, meloxicam, salicylamide, salicylic acid, desoxysulindac, tenoxicam, ketoralac, clonidine, flufenisal, salsalate, triethanolamine salicylate, aminopyrine, antipyrine, oxyphenbutazone, apazone, cintazone, flufenamic acid, clonixeril, clonixin, meclofenamic acid, flunixin, colchicine, demecolcine, allopurinal, oxypurinol, benzydamine hydrochloride, dimefadane, indoxole, intrazole, mimbane hydrochloride, paranylene hydrochloride, tetrydamine, benzindopyrine hydrochloride, fluprofen, ibufenac, naproxol, fenbufen, cinchophen, diflumidone sodium, fenamole, flutiazin, metazamide, letimide hydrochloride, nexeridine hydrochloride, octazamide, molinazole, neocinchophen, nimazole, proxazole citrate, tesicam, tesimide, tolmetin, triflumidate, fenamates (mefenamic acid, meclofenamic acid), nabumetone, celecoxib, etodolac, nimesulide, apazone, gold, tepoxalin; dithiocarbamate, or a combination thereof. Anti-inflammatory agents also include other compounds such as steroids, such as for example, fluocinolone, cortisol, cortisone, hydrocortisone, fludrocortisone, prednisone, prednisolone, methylprednisolone, triamcinolone, betamethasone, dexamethasone, beclomethasone, fluticasone interleukin-1 receptor antagonists, thalidomide (a TNF-α release inhibitor), thalidomide analogues (which reduce TNF-α production by macrophages), bone morphogenetic protein (BMP) type 2 or BMP-4 (inhibitors of caspase 8, a TNF-α activator), quinapril (an inhibitor of angiotensin II, which upregulates TNF-α), interferons such as IL-11 (which modulate TNF-α receptor expression), and aurin-tricarboxylic acid (which inhibits TNF-α), guanidinoethyldisulfide, or a combination thereof.

### 3. Steroid

In an embodiment, drug is a “steroid” include but not limited to 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, dexamethasone 21-acetate, dexamethasone 21-phosphate di-Na salt, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide or a combination thereof.

### 4. Antiviral Agent

In an embodiment, drug is an “antiviral agent”, a 5-substituted 2′-deoxyuridine analogue, a nucleoside analogue, a pyrophosphate analogue, a nucleoside reverse transcriptase inhibitor, a non-nucleoside reverse transcriptase inhibitor, a protease inhibitor, an integrase inhibitor, an entry inhibitor, an acyclic guanosine analogue, an acyclic nucleoside phosphonate analogue, a HCV NS5A/NS5B inhibitor, an influenza virus inhibitor, an interferon, an immunostimulatory agent, an agent for treatment of RSV, an agent for treatment of picornavirus, an agent for treatment of malaria, an agent for treatment of coronavirus, an agent for treatment of ebola virus, an agent for treatment of HCV, a NS5A inhibitor, an anti-HBV agent, an agent for treatment of HIV, a KRAS inhibitor, a proteasome inhibitor, a vaccine, an antibody, a polymerase inhibitor.

In an embodiment, drug is to treat HIV, HIV nucleoside reverse transcriptase translocation inhibitors, HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, capsid inhibitors, immune-based therapies, PI3K inhibitors, HIV antibodies, and bispecific antibodies, and “antibody-like” therapeutic proteins, and combinations thereof. In some embodiments, the one or more (e.g., one, two, three, or four; or one or two; or one to three; or one to four) additional therapeutic agents are selected from immunomodulators, immunotherapeutic agents, antibody-drug conjugates, gene modifiers, gene editors (such as CRISPR/Cas9, zinc finger nucleases, homing nucleases, synthetic nucleases, TALENs), and cell therapies such as chimeric antigen receptor T-cell, CAR-T (e.g., YESCARTA® (axicabtagene ciloleucel)), engineered T cell receptors, TCR-T, and combinations thereof.

In an embodiment, drug is a HIV protease inhibitor, for example but not limited to darunavir, atazanavir or similar.

### 5. Cytotoxic Agent

In an embodiment, drug is a cytotoxic drug used during chemotherapy that can be broken down into several main categories including alkylating agents, antimetabolites, anti-tumor antibiotics, topoisomerase inhibitors, and mitotic inhibitors. Cytotoxic anti-cancer drugs typically cause cell division to cease and thus affect healthy tissue as well as cancerous tissue. Alkylating agents stop cancer cell division by damaging the DNA of the cancer cell. Some common alkylating agents used to treat cancer are nitrogen mustards (e.g. cyclophosphamide (Cytoxan®; Cytoxan is a registered trademark of Baxter International), nitrosoureas, alkyl sulfonates, triazeines, and ethylenimines). Platinum drugs, such as cisplatin and carboplatin, work similarly to alkylating agents. Antimetabolites stop cancer cell division by inhibiting DNA and RNA synthesis. Some common antimetabolites used to treat cancer are 6-mercaptopurine, gemcitabine (Gemzar®; Gemzar is a registered trademark of Eli Lilly and Company), methotrexate and pemetrexed (Alimta®; Alimta is a registered trademark of Eli Lilly and Company). Topoisomerase inhibitors stop cancer cell division by inhibiting topoisomerase enzymes from separating the DNA for replication. Some common topoisomerase inhibitors are topotecan, irinotecan, etoposide, and teniposide. Mitotic inhibitors stop cancer cell division by inhibiting key cell division enzymes. Some common mitotic inhibitors are taxanes (e.g. paclitaxel (Taxol®; Taxol is a registered trademark of Bristol-Myers Squibb Company) and docetaxel (Taxotere®; Taxotere is a registered trademark of Aventis Pharma SA), epothilones, and vinca alkaloids.

### 6. Anti-Diabetic Agent

In an embodiment, the anti-diabetic agent is a stimulator of insulin release from pancreas such as but not limited to a sulfonylurea or a meglitinide. The sulfonylurea may be acetohexamide (DYMELOR), chlorpropamide (DIABINESE), tolbutamide (ORINASE, RASTINON), glipizide (GLUCOTROL, GLUCOTROL XL), glyburide (DIABETA; MICRONASE; GLYNASE), glimepiride (AMARYL), glisoxepid (PRO-DIABAN), glibenclamide (AZUGLUCON), glibomuride (GLUBORID), tolazamide, carbutamide, gliquidone (GLURENORM), glyhexamide, phenbutamide, tolcyclamide or gliclazide (DIAMICRON). The meglitinide may be Repaglinide (PRANDIN) or nateglinide (STARLIX).

In a further embodiment, the anti-diabetic agent is a glucosidase inhibitor such as but not limited to acarbose (PRECOSE, GLUCOBAY), miglitol (GLYSET, DIASTABOL) or voglibose.

In yet another embodiment, the anti-diabetic agent is an incretin or incretin analogue. The incretin or incretin analogue may be GLP-1, GIP, EXENATIDE or EXENATIDE LAR.

In still another embodiment, the anti-diabetic agent is a DPP-IV inhibitor selected from the group consisting of alanyl pyrrolidine, isoleucyl thiazolidine, and O-benzoyl hydroxylamine.

The agent is a prodrug of Glu-boroPro. For example, the agent may be a cyclic version of Glu-boroPro, an ester of Glu-boroPro, a boroxine molecule, or an alcohol precursor of Glu-boroPro.

In an embodiment, drug could be nerve growth factors, NAD+ precursors nicotinamide mononucleotide (NMN) and nicotinamide riboside (NR), agents that increase the level of NAD+ for use in preventing cramps, tremors, spasms, paralysis, neuromuscular paralysis, hearing loss, vision impairment, taste loss, improving or preventing decline in skills, gait, and coordination. In an embodiment, agents are the substances that regulate the functioning of brain and the wellbeing of nervous system of the body.

In an embodiment, drug can be, for example, insulin, luteinizing hormone-releasing hormone (LH-RH), somatostatin, or growth hormone releasing factor (GRF), and biologically active analogs thereof. The drug also can be a cytostatic compound, an analgesic compound, a hormone, or a vaccine.

In an embodiment, drug could be insulin, GLP-1 and at least a third compound. The third compound being selected to produce a synergistic effect in one or more of blood glucose reduction, blood glucose regulation, appetite control/suppression or other therapeutic effect of the insulin and GLP-1. Such synergistic effects in turn provide treatment for various conditions associated with diabetes (or other glucose regulating disorder) such as hyperglycemia, insulin resistance or hyperlipidemia. The glucose regulating compounds may include those which lower blood glucose, also known as hypoglycemic compounds, and those which raise blood glucose. They also include those which affect (e.g., reduce) glucose directly, and/or indirectly e.g., by causing the secretion of hormone which subsequently lowers glucose levels, agonize, or enhance the effect of glucose reducing hormones (as is the case for DDP4 inhibitors with incretins), reduce appetite (as is the case with, peptide YY) or all of the above. According to some embodiments, the other glucose regulating compound can be selected from the group consisting of glucagon, peptide YY, GIP, metformin, peptide YY, Dipeptidyl peptidase-4 (DPP4), DPP4 inhibitors, sodium/glucose co-transporter 2 (SGLT2) inhibitors along with their analogues and derivatives.

In some embodiments, drugs can be a class of type 2 diabetes drug that can improve blood sugar control and lead to weight loss. The examples of drugs that lead to weight loss and improved sugar control comprises GLP-1 receptor analogs, canagliflozin, ertugliflozin, dapagliflozin and empagliflozin. Examples of GLP-1 receptor agonists of this invention are exenatide, exenatide LAR, Albiglutide, Dulaglutide, Tirzepatide, Taspoglutide, Efpeglenatide, Semaglutide, Liraglutide, and Lixisenatide.

### 7. Antidepressant Agent

In an embodiment, drug treats depression. In an embodiment, drug is selected from (SSRIs), such as citalopram (Celexa), escitalopram oxalate (Lexapro), fluoxetine (Prozac), fluvoxamine (Luvox), paroxetine HCl (Paxil), and sertraline (Zoloft), (SNRIs), desvenlafaxine (Khedezla), desvenlafaxine succinate (Pristiq), duloxetine (Cymbalta), levomilnacipran (Fetzima), venlafaxine (Effexor), vortioxetine (Trentellix—formerly called Brintellix) or vilazodone (Viibryd), amitriptyline (Elavil), imipramine (Tofranil), nortriptyline (Pamelor), and doxepin (Sinequan).

In an embodiment, drugs are thought to affect mainly dopamine and norepinephrine such as bupropion (Wellbutrin). Monoamine oxidase inhibitors (MAOIs), such as isocarboxazid (Marplan), phenelzine (Nardil), selegiline (EMSAM), and tranylcypromine (Parnate).

In an embodiment, drugs are tetracyclic antidepressants that are noradrenergic and specific serotonergic antidepressants (NaSSAs), such as mirtazapine (Remeron). L-methylfolate (Deplin) has proven successful in treating depression. Other anti-anxiety medications include benzodiazepines, such as alprazolam (Xanax), clonazepam (Klonopin), diazepam (Valium), and lorazepam (Ativan). The drug buspirone (Buspar) is a unique serotonergic drug that is non-habit-forming and often used to treat generalized anxiety disorder. Some antiseizure medicines, such as gabapentin (Neurontin) or pregabalin (Lyrica) are sometimes used “off label” (without an official United States Food and Drug Administration (US FDA) indication) to treat certain forms of anxiety.

In an embodiment, drug is Aripiprazole (Abilify), Asenapine (Saphris), Cariprazine (Vraylar), Clozapine (Clozaril), Lurasidone (Latuda), Olanzapine (Zyprexa), Olanzapine/samidorphan (Lybalvi), Questiapine (Seroquel), Risperidone (Risperdal), Ziprasidone (Geodon) (Adderall, Adderall XR), methylphenidate (Daytrana) patch, dextroamphetamine (Dexedrine), lisdexamfetamine (Vyvanse), and methylphenidate (Concerta, Quillivant XR, Ritalin), dextroamphetamine-amphetamine (Mydayis).

### 8. Nonstimulant Agent

In an embodiment, drug is an alpha agonist, are nonstimulant medicines that are also sometimes used to treat ADHD. Examples include but not limited to clonidine (Catapres), guanfacine (Intuniv), Atomoxetine (Strattera) and buproprion (Wellbutrin).

In an embodiment, drugs are “statin” to lower cholesterol level. For example, but not limited to Lipitor, Livalo, Mevacor or Altocor, Zocor, Pravachol, Lescol and Crestor.

In an embodiment, drug delivered is Hydromorphine, Lidocaine, Buprenorphine, Isoniazid, Pyrazinamide, Risperidone, Pilocarpine, Alginic acid, Fluocinolone, Ganciclovir, Goserelin, Leuprolide, Carmustine (BCNU), Paclitaxel, Histrelin, Levonorgestrel, Estradiol, Etonogestrel, Ethinyl estradiol, Etonogestrel and Gemcitabine.

### 9. Anti Arthritis and Anti-Rheumatic Agent

In an embodiment, drug may be to treat arthritis that may include disease-modifying anti-rheumatic drugs (DMARDs), Biologic response modifiers (a type of DMARD), Glucocorticoids, Nonsteroidal anti-inflammatory medications (NSAIDs), Analgesics (painkillers). Few examples of DMARD are but not limited to hydroxychloroquine sulfate, leflunomide, methotrexate, tofacitinib, baricitinib, Upadacitinib. Few examples of biologic response modifiers are but not limited to abatacept, adalimumab, adalimumab-atto, anakinra, etanercept, etanercept-szzs, infliximab, Infliximab-abda, infliximab-dyyb, rituximab, rituximab-abbs, rituximab-pvvr, infliximab-dyyb, golimumab, certolizumab pegol, tocilizumab, sarilumab.

### 10. Blood Pressure Related Agent

In an embodiment, drug may treat blood pressure, which may include Angiotensin-converting enzyme (ACE) inhibitors angiotensin II receptor blockers (ARBs), Diuretics, Beta-blockers, Calcium channel blockers, Alpha-blockers, Alpha-agonists, and Renin inhibitors.

U.S. Pat. No. 9,265,733B2, U.S. Ser. No. 11/439,709B2, U.S. Pat. Nos. 5,616,123A, 9,402,807B2 and US20060063719A1 are incorporated by reference in their entirety.

### 11. Autoimmune Disease-Related Drugs

In an embodiment, drug may be to treat autoimmune diseases and allergy or to inhibit anti-drug antibody production or to induce antigen specific immune tolerance by applying the combination of antigen and immunosuppressive agent/drug. The term “immunosuppressant” commonly refers to a drug for suppressing an immune response in a living body. Examples of immunosuppressants may include calcineurin inhibitors including glucocorticoid, cyclosporine, tacrolimus (FK506), pimecrolimus, and ISA(TX)247, rapamycin, a Type IV PDE inhibitor, mycophenolate mofetil, dexamethasone, and the like, but the present invention is not limited thereto. For example, all kinds of known immunosuppressants may be used herein.

Also, one immunosuppressant may be used alone, or two or more immunosuppressants may be used in combination. Preferably, at least one selected from the group consisting of cyclosporine, tacrolimus, dexamethasone, and pimecrolimus may be used as the immunosuppressant. The immunosuppressive agent/drug (immunosuppressants) suitable for the current application include but are not limited to, statins; mTOR inhibitors, such as rapamycin or a rapamycin analog; TGF-β signaling agents; TGF-β receptor agonists; TLR (toll like receptor) inhibitors; Pattern recognition receptor inhibitors; NOD-like receptors (NLR) inhibitors; RIG-I-like receptors inhibitors; NOD2 inhibitors; histone deacetylase inhibitors, such as Trichostatin A; corticosteroids; inhibitors of mitochondrial function, such as rotenone; P38 inhibitors; NF-κβ inhibitors, such as 6Bio, Dexamethasone, TCPA-1, IKK VII; adenosine receptor agonists; prostaglandin E2 agonists (PGE2), such as Misoprostol; phosphodiesterase inhibitors, such as phosphodiesterase 4 inhibitor (PDE4), such as Rolipram; proteasome inhibitors; kinase inhibitors; G-protein coupled receptor agonists; G-protein coupled receptor antagonists; glucocorticoids; retinoids; cytokine inhibitors; cytokine receptor inhibitors; cytokine receptor activators; peroxisome proliferator-activated receptor antagonists; peroxisome proliferator-activated receptor agonists; histone deacetylase inhibitors; calcineurin inhibitors; phosphatase inhibitors; PI3 KB inhibitors, such as TGX-221; autophagy inhibitors, such as 3-Methyladenine; aryl hydrocarbon receptor inhibitors; proteasome inhibitor I (PSI); and oxidized ATPs, such as P2X receptor blockers. Immunosuppressants also include IDO, vitamin D3, cyclosporins, such as cyclosporine A, aryl hydrocarbon receptor inhibitors, resveratrol, azathiopurine (Aza), 6-mercaptopurine (6-MP), 6-thioguanine (6-TG), FK506, sanglifehrin A, salmeterol, mycophenolate mofetil (MMF), aspirin and other COX inhibitors, niflumic acid, estriol and triptolide, siglec ligand such as sialic acid and its derivative including poly sialic acid sialic acid-lipid conjugate. In embodiments, the immunosuppressant may comprise any of the agents provided herein. The immunosuppressant can be a compound that directly provides the immunosuppressive (e.g., tolerogenic) effect on APCs or it can be a compound that provides the immunosuppressive (e.g., tolerogenic) effect indirectly (i.e., after being processed in some way after administration). Immunosuppressants, therefore, include prodrug forms of any of the compounds provided herein.

Pharmaceutical agents that are used for long-term as medication may be used via the devices described in this application. Examples of pharmaceutical agents comprise one or more of the following: a antihypertensive agent, analgesic, antidepressant, opioid agonist, anesthetic, antiarrhythmic, antiarthritic, antispasmodic, ACE inhibitor, decongestant, antibiotic, antihistamine, anti-anginal, diuretic, anti-hypotensive agent, anti-Parkinson agent, bronchodilator, oxytocic agent, anti-diuretic, anti-hyperglycemic, antineoplastic and/or immunosuppresent agent, antiemetic, anti-infective, antifungal, antiviral, antimuscarinic, antidiabetic agent, antiallergy agent, anxiolytic, sedative, antipsychotic, bone modulating agent, cardiovascular agent, cholesterol lowering drug, antimalarial, antiepileptic, antihelminthic, agent for smoking cessation, cough suppressant, expectorant, mucolytic, nasal decongestant, dopaminergic, gastrointestinal agent, muscle relaxant, neuromuscular blocker, parasympathomimetic, prostaglandin, stimulant, anorectic, thyroid or antithyroid agent, hormone, antimigrane agent, antiobesity, and/or non-steroidal anti-inflammatory agent. Further, the pharmaceutical agent may be one or more of the following: dihydroergotamine, fentanyl, sufentanil, lidocaine, alfentanil, lofentanil, carfentanil, pentobarbital, buspirone, ergotamine, bisphosphonate, alendronic acid, nalbuphine, bupropion, metformin, diethylcarbamazine, tramadol, heparin or a heparin derivative, amoxicillin, gabapentin, econazole, aspirin, prostaglandin, methylsergide, ergonovine, endorphins, enkephalins, oxytocin, opiates, heparin and its derivatives, clorazepic acid, barbiturate, albuterol, atropine, scopolamine, selegiline, timolol, nicotine, cocaine, novocaine, amphetamines, caffeine, methylphenidate, chlorpromazine, ketamine, epinephrine, estropipate, naloxone, naltrexone, furosemide, labetalol, metoprolol, nadolol, isoproterenol, terbutaline, sumatriptan, bupivacaine, prilocalne, loratadine, chloropheniramine, clonidine, or tetracaine. In one example, the pharmaceutical agent is nicotine.

### 12. Weight Loss/Obesity Loss Related Drugs

In an embodiment, drug can reduce excess body weight that consequentially help in controlling obesity. In an embodiment, the drug may cause average body weight reduction by 2%, 3%, 5%, 6%, 7%, 8%, 9%, 10%, 12%, 15%, 20%, 30%, 40%, 50% or more. Few examples of drugs, without any limitation are orlistat®, liraglutide®, Ozempic®, Wegovy®, Ozempic®, and Rybelsus®, tirzepatide®, semaglutide, or etc.

In an embodiment, semaglutide is a glucagon-like peptide-1 (GLP-1) analog. It could be used for the treatment of type 2 diabetes (oral semaglutide and subcutaneous semaglutide) and also for reducing the risk of cardiovascular events in people with type 2 diabetes and cardiovascular disease.

In an embodiment, semaglutide may be present in a formulation having a disodium phosphate dihydrate buffer and propylene glycol, wherein said propylene glycol is present in said formulation in a final concentration of from about 1 mg/ml to about 100 mg/ml and wherein said formulation has a pH of from about 7.0 to about 10.0 (as disclosed in U.S. Pat. No. 8,114,833B2, which is incorporated by reference in its entirety).

In an embodiment, GLP-1 analog may have modifications such as but not limited to: wherein at least one amino acid residue of the parent peptide has a lipophilic substituent attached; wherein GLP-1(7-35) and GLP-1(7-36) derivatives which have a lipophilic substituent attached to the C-terminal amino acid residue, acylated GLP-1 analogs (as disclosed in U.S. Pat. No. 8,536,122B2, which is incorporated by reference in its entirety), double acylated GLP-1 derivates such as acylated at K27 and at another K residue of the peptide (as disclosed in U.S. Pat. No. 9,266,940B2, which is incorporated by reference in its entirety) a non-proteogenic amino acid residue in positions 7 and/or 8 relative to the sequence GLP-1(7-37), which is acylated with a moiety to the lysine residue in position 26, and where said moiety comprises at least two acidic groups, wherein one acidic group is attached terminally (as disclosed in U.S. Pat. No. 8,129,343B2, which is incorporated by reference in its entirety).

An embodiment provides a GLP-1 analog according to the embodiment above, wherein said GLP-1 analog comprises imidazopropionyl7, α-hydroxy-histidine7 or N-methyl-histidine7, D-histidine7, desamino-histidine7, 2-amino-histidine7, β-hydroxy-histidine7, homohistidine7, acetyl-histidine7, α-fluoromethyl-histidine7, α-methyl-histidine7, 3-pyridylalanine7, 2-pyridylalanine7 or 4-pyridylalanine7.

An embodiment provides a GLP-1 analog according to any one of the embodiments above, wherein said GLP-1 analog comprises a substitution of the L-alanine in position 8 of the GLP-1 (7-37) sequence for another amino acid residue.

An embodiment provides a GLP-1 analog according to the embodiment above, wherein said GLP-1 analog comprises Aib8, Gly8, Val8, Ile8, Leu8, Ser8, Thr8, (1-aminocyclopropyl) carboxylic acid, (1-aminocyclobutyl) carboxylic acid, (1-aminocyclopentyl) carboxylic acid, (1-aminocyclohexyl) carboxylic acid, (1-aminocycloheptyl) carboxylic acid, or (1-aminocyclooctyl) carboxylic acid.

In one embodiment, the GLP-1 peptide is a DPPIV protected GLP-1 peptide. In one embodiment the GLP-1 peptide is DPPIV stabilized (as disclosed in U.S. Ser. No. 10/335,462B2, which is incorporated by reference in its entirety.)

In an embodiment, drug may agonize receptors for both human glucose-dependent insulinotropic polypeptide (GIP) and glucagon-like peptide-1 (GLP-1) and may be useful for treating type 2 diabetes mellitus (T2D) (as disclosed in U.S. Pat. No. 9,474,780B2, which is incorporated by reference in its entirety.)

In an embodiment, semaglutide may be provided along with vitamins such as vitamin B12 or other hormones.

In an embodiment, drug combination may be (1) a GLP-1R agonist and (2) an ACC inhibitor or a DGAT2 inhibitor, or a KHK inhibitor or FXR agonist (as described in US20220387402A1, which is incorporated by reference in its entirety.)

In one embodiment, the drug comprises a high potency drug. Some examples of high potency drugs include Fentanyl (an opioid used for pain management), Carfentanil (an opioid used as an anesthetic for large animals), Lysergic acid diethylamide (a hallucinogenic drug), Methamphetamine (a potent stimulant drug), Dimethyltryptamine (a hallucinogenic drug), Heroin (an opioid drug derived from morphine), Cocaine (a stimulant derived from the coca plant), 3,4-methylenedioxymethamphetamine (a psychoactive drug), and Potent antineoplastic drugs (used in chemotherapy for cancer treatment).

In another embodiment, the drug is dosed in milligrams per day. Some examples of drugs dosed in milligrams per day are Aspirin (Acetylsalicylic Acid), Ibuprofen, Paracetamol (Acetaminophen), Metformin, Ciprofloxacin, Lisinopril, Atorvastatin, Sertraline, and Lorazepam.

In yet another embodiment, the drug is dosed at about 1 milligram per day. Some examples of drugs dosed at about 1 milligram per day are Risperidone, Finasteride, Dutasteride, Estradiol (or Estrogen), and Anastrozole.

In yet another embodiment, the drug is dosed in less than 1 milligram per day. Some examples of drugs dosed at less than 1 milligram per day are Levothyroxine, Warfarin, Alprazolam, Clonazepam, Buprenorphine, and Donepezil.

In yet another embodiment, the drug is dosed in less than 1 milligram per week. An example of a drug dosed at about less than 1 milligram per week is Methotrexate.

In yet another embodiment, the drug is dosed at 2 milligrams per week.

In yet another embodiment, the drugs are injectables. Some examples of injectable drugs are Insulin, Epinephrine, Vaccines, Heparin, Methotrexate, Cyanocobalamin, Morphine, Enoxaparin, and Adalimumab.

In yet another embodiment, the drugs are dosed once weekly by injection. Examples of such drugs are Methotrexate, Prolia (Denosumab), Bydureon (Exenatide), Ozempic (Semaglutide), and Enbrel (Etanercept).

In yet another embodiment, the drugs are dosed 1-3 milligrams via injection. Some examples of such drugs are Paliperidone palmitate (Invega Sustenna/Xeplion), Aripiprazole lauroxil (Aristada), Risperidone microspheres (Perseris), Liraglutide (Victoza, Saxenda), Leuprolide acetate (Lupron), Naltrexone/bupropion (Contrave). Lanreotide (Somatuline Depot), and Degarelix (Firmagon).

In yet another embodiment, the drugs are dosed 1-3 milligrams via injection. Some examples of such drugs are Epoetin alfa (Epogen, Procrit), Pegfilgrastim (Neulasta), Ibandronate (Boniva), Palonosetron (Aloxi), and Octreotide (Sandostatin).

### C. Drug Formulations to be Used in the Implantable Device

### 1. Suspension Formulation

In an embodiment, the drug is provided in a suspension formulation comprising: (a) a particle formulation comprising said drug; and (b) a vehicle formulation, wherein the particle formulation is dispersed in the vehicle.

In an embodiment, particle formulation typically further includes one or more additional components, for example, one or more stabilizers (e.g., carbohydrates, antioxidants, amino acids, and buffers). Such concentrated particle formulations can be combined with a suspension vehicle to form suspension formulations. The suspension formulation comprises (i) a non-aqueous, single-phase vehicle, comprising one or more polymers and one or more solvent, wherein the vehicle exhibits viscous fluid characteristics, and (ii) a highly concentrated drug particle formulation.

In an embodiment, the suspension formulation may further comprise a particle formulation comprising a drug and one or more stabilizers selected from the group consisting of carbohydrates, antioxidants, amino acids, buffers, and inorganic compounds. The suspension formulation further comprises a non-aqueous, single-phase suspension vehicle comprising one or more polymers and one or more solvents. The suspension vehicle typically exhibits viscous fluid characteristics, and the particle formulation is dispersed in the vehicle. The particle formulation is uniformly dispersed in the vehicle.

### 2. Particle Formulation

In another embodiment, the suspension formulation comprises a particle formulation comprising a drug, a disaccharide (e.g., sucrose), methionine, and a buffer (e.g., citrate), and a non-aqueous, single-phase suspension vehicle comprising one or more pyrrolidone polymer (e.g., polyvinylpyrollidone) and one or more solvent, e.g., lauryl lactate, lauryl alcohol, benzyl benzoate, or mixtures thereof. The ratio of the drug to sucrose+methionine is typically about 1/20, about 1/10, about 1/5, about 1/2, about 2/1, about 5/1, about 10/1, or about 20/1, preferably between about 1/5 to 5/1, more preferably between about 1/3 to 3/1. The particle formulation is preferably a particle formulation prepared by spray drying and has a low moisture content, preferably less than or equal to about 10 wt %, more preferably less or equal to about 5 wt %. Alternatively, the particle formulation can be lyophilized.

### 2.1 Buffer

In an embodiment, the particle formulations of the present invention may further comprise a buffer, for example, selected from the group consisting of citrate, histidine, succinate, and mixtures thereof.

### 2.2 Inorganic Compound

In an embodiment, the particle formulations of the present invention may further comprise an inorganic compound, for example, selected from the group consisting of NaCl, Na₂SO₄, NaHCO₃, KCl, KH₂PO₄, CaCl₂, and MgCl₂.

### 2.3 Stabilizer

In an embodiment, one or more stabilizers in the particle formulations may comprise, for example, a carbohydrate selected from the group consisting of lactose, sucrose, trehalose, mannitol, cellobiose, and mixtures thereof. The one or more stabilizers in the particle formulations may comprise, for example, an antioxidant selected from the group consisting of methionine, ascorbic acid, sodium thiosulfate, ethylenediaminetetraacetic acid (EDTA), citric acid, cysteins, thioglycerol, thioglycolic acid, thiosorbitol, butylated hydroxanisol, butylated hydroxyltoluene, and propyl gallate, and mixtures thereof. The one or more stabilizers in the particle formulations may comprise an amino acid.

### 2.4 Solvent

In one embodiment, the solvent of the suspension vehicle of the present invention is selected from the group consisting of lauryl lactate, lauryl alcohol, benzyl benzoate, and mixtures thereof. An example of a polymer that can be used to formulate the suspension vehicle is a pyrrolidone (e.g., polyvinylpyrrolidone). In a preferred embodiment, the polymer is a pyrrolidone and the solvent is benzyl benzoate.

### 2.5 Polymer to Solvent

In an embodiment, proportions of polymer to solvent in the suspension vehicle may be varied, for example, the suspension vehicle may comprise about 40 wt % to about 80 wt % polymer(s) and about 20 wt % to about 60 wt % solvent(s). Preferred embodiments of a suspension vehicle include vehicles formed of polymer(s) and solvent(s) combined at the following ratios: about 25 wt % solvent and about 75 wt % polymer; about 50 wt % solvent and about 50 wt % polymer; and about 75 wt % solvent and about 25 wt % polymer.

In an embodiment, the suspension formulation typically has an overall moisture content less than about 10 wt % and in a preferred embodiment less than about 5 wt %.

In an embodiment, particle formulations are preferably chemically and physically stable for at least one month, preferably at least three months, more preferably at least six months, more preferably at least 12 months at delivery temperature. The delivery temperature is typically a normal human body temperature, for example, about 37° C., or slightly higher, for example, about 40° C. Further, particle formulations are preferably chemically and physically stable for at least three months, preferably at least six months, more preferably at least 12 months, at storage temperature. Examples of storage temperatures include refrigeration temperature, for example, about 5° C., or room temperature, for example, about 25° C.

In a preferred embodiment, when the particles are incorporated in a suspension vehicle, they do not settle in less than about three months at delivery temperature. Generally speaking, smaller particles tend to have a lower settling rate in viscous suspension vehicles than larger particles. Accordingly, micron- to nano-sized particles are typically desirable. In an embodiment of the particle formulation for use in an implantable osmotic delivery device, wherein the delivery orifice diameter of the implant is in a range of, for example, about 0.1 to about 0.5 mm, particle sizes may be preferably less than about 50 microns, more preferably less than about 10 microns, more preferably in a range from about 3 to about 7 microns.

### 2.6 Excipient

In an embodiment, excipients with higher Tg may be included in the formulation to improve stability, for example, sucrose (Tg=75° C.) and trehalose (Tg=110° C.). Preferably, particle formulations are formable into particles using processes such as spray drying, lyophilization, desiccation, milling, granulation, ultrasonic drop creation, crystallization, precipitation, or other techniques available in the art for forming particles from a mixture of components. The particles are preferably substantially uniform in shape and size. Uniform shape and size of the particles typically helps to provide a consistent and uniform rate of release from such a delivery device; however, a particle preparation having a non-normal particle size distribution profile may also be used. In one embodiment, the particles are sized such that they can be delivered via an implantable drug delivery device. The uniform shape and size of the particles typically helps to provide a consistent and uniform rate of release from such a delivery device; however, a particle preparation having a non-normal particle size distribution profile may also be used. For example, in a typical implantable osmotic delivery device having a delivery orifice, the size of the particles is less than about 30%, preferably is less than about 20%, more preferably is less than about 10% of the diameter of the delivery orifice. In an embodiment of the particle formulation for use with an osmotic delivery device, wherein the delivery orifice diameter of the implant is in a range of, for example, about 0.1 to about 0.5 mm, particle sizes may be preferably less than about 50 microns, more preferably less than about 10 microns, more preferably in a range from about 3 to about 7 microns. In one embodiment, the orifice is about 0.25 mm (250 microns), and the particle size is approximately 3-5 microns.

The suspension vehicle of the present invention is a viscous fluid or gel-like material. As it is used herein, the term “viscous fluid” refers to a flowable fluid, gel or gel-like material having a viscosity within a range of about 500 to 1,000,000 poise as measured by a parallel plate rheometer at a shear rate of 10-4/sec and 37° C. The term “viscous gel” includes Newtonian and non-Newtonian materials. Preferred are gels with a viscosity of about 1,000 to 30,000 poise as measured by a parallel plate rheometer at a shear rate of 10-4/sec and 37° C. Viscous suspension vehicles allow the creation of beneficial agent suspensions capable of delivering beneficial agent at a substantially uniform rate over prolonged periods of time as the suspension is expelled from an implantable delivery device at a controlled rate. In an embodiment, suspension vehicle typically has a viscosity, at 33° C., between about 5,000 to about 30,000 poise, preferably between about 8,000 to about 25,000 poise, more preferably between about 10,000 to about 20,000 poise. In one embodiment, the suspension vehicle has a viscosity of about 15,000 poise, plus or minus about 3,000 poise, at 33° C.

In an embodiment, suspension vehicle of the present invention may include an amount of other excipients or adjuvants, such as surfactants, antioxidants, stabilizers, and viscosity modifiers.

In an embodiment, a particle formulation may be considered chemically stable if less than about 25%, preferably less than about 20%, more preferably less than about 15%, more preferably less than about 10%, and more preferably less than about 5%, breakdown products of the peptide particles are formed after about three months, preferably after about six months, preferably after about 12 months, at delivery temperature and after about six months, after about 12 months, and preferably after about 24 months, at storage temperature.

In an embodiment, a particle formulation may be considered physically stable if less than about 10%, preferably less than about 5%, more preferably less than about 3%, more preferably less than 1%, aggregates of the peptide particles are formed after about three months, preferably after about six months, at delivery temperature and about 6 months, preferably about 12 months, at storage temperature.

### D. Device for Controlled Delivery Comprising Micro or Nano Beads

In one embodiment, the present disclosure relates to a device for controlled delivery of drugs, comprising a micro bead or nano beads that contains a drug, wherein the beads are embedded in a flowable mixture such as a liquid, an emulsion or polymer that are approved by the Food and Drug Administration (FDA) for injection into a human body, e.g., a biodegradable sol-gel or biodegradable thermoplastic polymer, including a biodegradable foam that can be squeezed out of the implant.

In one embodiment, the present disclosure relates to a device for controlled delivery of drugs, comprising a micro bead or nano beads that contain a drug, wherein the device can be implanted into specific organs or underneath the skin for an effective local or systemic delivery of drug.

In one embodiment, the present disclosure relates to a device for controlled delivery of drugs, comprising a micro or nano beads that contains drug, wherein the device provides sustained drug delivery for a prolonged period of time. Preferably, the time period of ranges is from about 1 week to about 5 years. Preferably, the time period of ranges are from about 1 month to about 3 years. In one aspect of this embodiment, the device is pre-loaded in a needle supplied with a disposable applicator.

In one embodiment, the present disclosure relates to a device for controlled delivery of drugs, comprising a micro or nano beads that contains drug, wherein the device is suitable for delivery of small molecules having molecular weight less than 500 as well as large biologics entities like peptides, antibodies, and nucleic acid analogs such as modified RNA, small interfering RNA, anti-sense DNA or fragments thereof.

In another embodiment, the present disclosure relates to a device for controlled delivery of drugs, comprising a micro or nano beads that contains drug, wherein a top coating can be applied on the beads to delay release of the active agent. In another embodiment, a top coating can be used for the delivery of a second active agent. A layered coating, comprising respective layers of fast- and slow-hydrolyzing polymer, can be used to stage release of the active agent or to control release of different active agents placed in the different layers. Polymer blends may also be used to control the release rate of different active agents or to provide a desirable balance of coating characteristics (e.g., elasticity, toughness) and drug delivery characteristics (e.g., release profile). Polymers with differing solvent solubilities can be used to build-up different polymer layers that may be used to deliver different active agents or to control the release profile of active agents.

The amount of an active agent present depends upon the particular active agent employed, and medical condition being treated. In one embodiment, the active agent is present in an effective amount. In another embodiment, the amount of the active agent represents from about 0.01% to about 60% of the coating by weight. In another embodiment, the amount of the active agent represents from about 0.01% to about 40% of the coating by weight. In another embodiment, the amount of the active agent represents from about 0.1% to about 20% of the coating by weight.

Another embodiment relates to a device comprising micro or nano beads having a shell comprising a first material and a second material, wherein the second material comprises a biodegradable material; a core comprising a pharmaceutically effective composition, the core being enclosed by the shell; wherein the first material is distributed in the biodegradable material; wherein the first material is configured to create holes in the shell; wherein the holes allow the pharmaceutically effective composition to be released to the exterior of the shell through the holes. In one embodiment, the shell could be made by polymerizing a silica-functionalized monomer to form a silica-containing biodegradable polymer shell.

Preferably, the first material comprises a metal-containing material that can be heated to form the holes or a biodegradable material that degrades over time. Preferably, the metal-containing material is configured to be heated under radiation, before or after implanting or attaching the device in or on a body of a subject, to form the holes. Preferably, the metal-containing material comprises metallic particles. Preferably, the metallic component comprises an iron-containing material or an iron-containing polymer. Preferably, the metallic particles comprise iron-containing particles or an iron-containing polymer. Preferably, the first material comprises a biodegradable material. Preferably, the first and second materials comprise polymers. Preferably, the first material comprises polylactic acid (PLA) or an iron-containing polymer and the second biodegradable material comprises poly ε-caprolactone (PCL). Preferably, the core comprises an emulsion or beads of the pharmaceutically effective composition and a polymer.

Preferably, the pharmaceutically effective composition comprises a targeting material or targeting molecule that binds to a certain organ, object, or a specific site within a body of a subject.

The implantable device of the embodiments herein can be implanted into specific organs, such as vagina, or underneath the skin for an effective local or systemic delivery of the pharmaceutical agent. In one embodiment, the present disclosure provides a device for controlled delivery of drugs, comprising a micro or nano beads that contain drug.

The beads used in the implantable device of the embodiments herein can be made by microfluidics. Microfluidics-based technology enables precise control and manipulation of fluids constrained to micron-sized capillaries. Advantages of microfluidics include reduced sample size and reagent consumption, short processing times, enhanced sensitivity, real-time analysis, and automation. More specifically, drop-based microfluidics allow for the creation of micron-sized emulsions that can hold discrete picoliter volumes, with drop-making frequencies of greater than 2,000 drops per second (2 kHz).

Soft lithography techniques could be employed to fabricate microfluidic devices for bead fabrication. For example, in one embodiment, AutoCAD software was used to generate a UV photomask containing micron-sized capillaries of desired structure and dimension. A silicon wafer was coated with UV photoresist, on which the photomask was placed. After UV exposure, the silicon wafer was further developed with propylene glycol monomethyl ether acetate (PGMEA) to generate a positive resist with the desired channels exposed. Polydimethylsiloxane (PDMS) was poured atop the positive resist and incubated at 65° C. overnight. After removing the PDMS (now a negative resist with the desired channels) from the silicon wafer, the inlets were punched and the PDMS was bonded to glass via plasma-activated bonding. The devices were treated with hydrophobic Aquapel to prevent the wetting of channels during drop formation. The device for droplet formation is disclosed in U.S. Patent Publication 20120222748, titled “Droplet creation techniques,” which is incorporated herein, in relevant parts for the purposes of written description, by reference in its entirety.

Additional U.S. Patents and Publications related to droplet formation are incorporated herein, in relevant parts for the purposes of written description, by reference in their entirety are:

U.S. Pat. No. 7,776,927 B2—This is a patent that broadly describes methods of droplet generation and describes some potential uses in drug delivery.

US20120141589 A1—This patent describes some compounds (such as CaCO₃) with which the microfluidic emulsions could be made depending on the drug encapsulated in the emulsion, droplets, and beads.

US20130202657—This publication describes a microfoam for drug delivery. Such a microfoam could be incorporated as the foam or mesh in the implantable device of the embodiments herein.

U.S. Pat. No. 6,858,220 B2—This patent discloses an implantable biocompatible microfluidic drug delivery system using only channels, but not microbeads containing a drug.

US20130035574, US20130035660—These publications describe the actual chip/patch rather than the microbeads. However, the publications use microfluidics as well as scaffolding for drug delivery.

U.S. Pat. No. 7,560,036 B2—This patent describes in detail the fabrication of the surface substrate and uses microneedles for drug delivery.

The drug containing beads could be made from droplets, for example, formed in accordance with the droplet creation techniques disclosed in U.S. Patent Publication US20120222748, for the implantable device of the embodiments by crosslinking biodegradable polymer of the shell of the drug containing beads. The crosslinking density of the biodegradable polymer of the shell could be varied such that even for the same shell thickness, the drug containing beads with low crosslinking density would rupture before the drug containing beads with high crosslinking density when the drug containing beads are exposed to blood serum or any other bodily fluid, for example.

The drug containing beads in the embodiments herein can have an inner core, which could be hollow or solid or porous, containing an active pharmaceutical ingredient, an optional intermediate coating substantially surrounding the inner core, and an outer coating substantially surrounding the optional intermediate coating comprising a pH independent polymer such as that disclosed in U.S. Patent Publication 20080187579, titled “Extended-release dosage form,” which is incorporated herein, in relevant parts for the purposes of written description, in its entirety. The implantable device of the embodiments herein could have two or more bead populations wherein each of the bead populations has a different drug release profile. The method of preparing an extended release dosage composition comprising one or more bead populations is disclosed in U.S. Patent Publication 20080187579, with an additional requirement that the beads are made of biodegradable material such as a biodegradable polymer.

In one embodiment, the present disclosure relates to a device for controlled delivery of drugs, comprising a micro or nano beads that contains drug, wherein the beads comprise a biocompatible, cross-linked, biodegradable material, collagen, fibronectin, elastin, hyaluronic acid, or a mixture thereof.

In one embodiment the biodegradable polymer material for the bead and foam and/or any other material of the device may include polyglycolic acid (“PGA”), polylactic acid (“PLA”), polycaprolactic acid (“PCL”), poly-p-dioxanone (“PDO”), PGA/PLA copolymers, PGA/PCL copolymers, PGA/PDO copolymers, PLA/PCL copolymers, PLA/PDO copolymers, PCL/PDO copolymers, or combinations thereof.

In another embodiment, the biodegradable polymer material may include polycarbonate polyurethanes, polycarbonate urea-urethanes, polyether polyurethanes, poly(carbonate-co-ether) urea-urethanes, polysiloxanes, and the like.

The implantable device could include a device such as a monitor/transmitter with the ability to detect blood glucose levels, sense hormonal levels, and/or detect body temperature. The implantable device could include a device such as a monitor/transmitter with an ability to communicate to the sensors/detectors that are connected to a smartphone, an ability to transmit data to iCloud, and/or an ability to sense appetite sensing hormones.

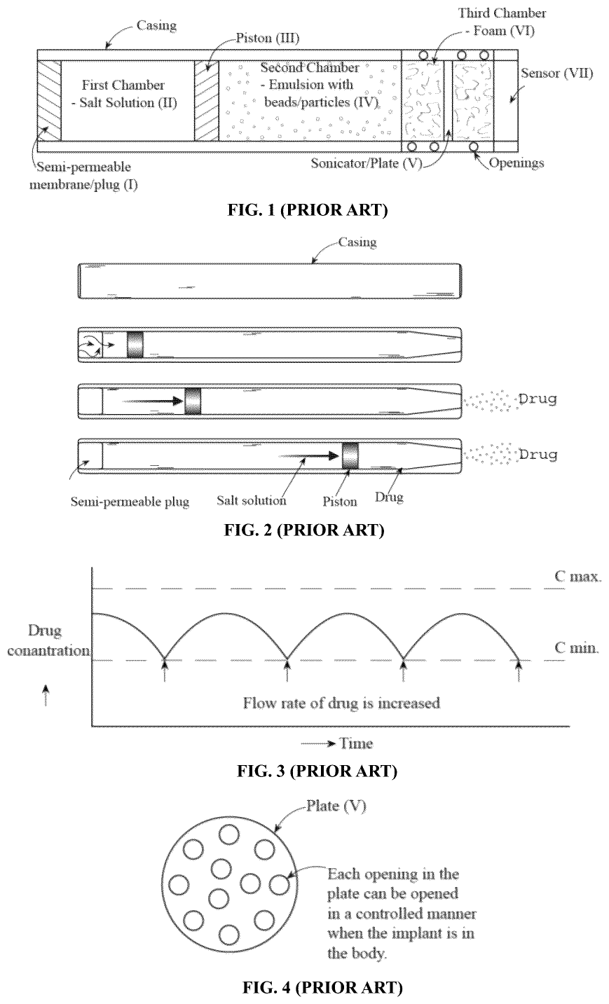

FIG. 1 is a schematic diagram showing the implantable device based on an osmotic pump delivery system, with several features explained below. An osmotic pump delivery is disclosed in some of the following US patents and applications of Intarcia Therapeutics, Inc., which are incorporated herein, in relevant parts for the purposes of written description, by reference in their entirety:

| Title | Application | Publication | Patent |
|---|---|---|---|
| DEVICES, FORMULATIONS, AND METHODS | 12/378,341 | 20090202608 | 8,343,140 |
| FOR DELIVERY OF MULTIPLE BENEFICIAL | Feb. 12, 2009 | Aug. 13, 2009 | Jan. 1, 2013 |
| AGENTS | | | |
| TWO-PIECE, INTERNAL-CHANNEL OSMOTIC | 13/601,939 | 20120330282 | 8,367,095 |
| DELIVERY SYSTEM FLOW MODULATOR | Aug. 31, 2012 | Dec. 27, 2012 | Feb. 5, 2013 |
| SUSTAINED DELIVERY OF AN ACTIVE | 13/645,124 | 20130035669 | 8,535,701 |
| AGENT USING AN IMPLANTABLE SYSTEM | Oct. 4, 2012 | Feb. 7, 2013 | Sep. 17, 2013 |
| RAPID ESTABLISHMENT AND/OR | 13/645,422 | 20130030417 | |
| TERMINATION OF SUBSTANTIAL STEADY- | Oct. 4, 2012 | Jan. 31, 2013 | |
| STATE DRUG DELIVERY | | | |
| OSMOTIC DELIVERY SYSTEMS AND PISTON | 12/930,950 | 20110166554 | 8,801,700 |
| ASSEMBLIES FOR USE THEREIN | Jan. 19, 2011 | Jul. 7, 2011 | Aug. 12, 2014 |
| SELF ADJUSTABLE EXIT PORT | 09/045,944 | | 5,997,527 |
| | Mar. 23, 1998 | | Dec. 7, 1999 |
| OSMOTIC DELIVERY SYSTEM AND METHOD | 8/970,530 | | 6,132,420 |
| FOR ENHANCING START-UP & | Nov. 14, 1997 | | Oct. 17, 2000 |
| PERFORMANCE OF OSMOTIC DELIVERY | | | |
| SYSTEMS | | | |
| IMPLANTER DEVICE FOR SUBCUTANEOUS | 09/217,824 | | 6,190,350 |
| IMPLANTS | Dec. 22, 1998 | | Feb. 20, 2001 |
| OSMOTIC DELIVERY SYSTEM | 09/121,878 | | 6,287,295 |
| SEMIPERMEABLE BODY ASSEMBLY | Jul. 24, 1998 | | Sep. 11, 2001 |
| RATE CONTROLLING MEMBRANES FOR | 09/213,213 | | 6,375,978 |
| CONTROLLED IMPLANTS | Dec. 17, 1998 | | Apr. 23, 2002 |
| VALVE FOR OSMOTIC DEVICES | 09/748,099 | | 6,508,808 |
| | Dec. 21, 2000 | | Jan. 21, 2003 |
| OSMOTIC DELIVERY SYSTEM FLOW | 09/122,073 | | 6,524,305 |
| MODULATOR APPARATUS AND METHOD | Jul. 24, 1998 | | Feb. 25, 2003 |
| OSMOTIC DELIVERY SYSTEM HAVING | 09/472,600 | | 6,544,252 |
| SPACE EFFICIENT PISTON | Dec. 27, 1999 | | Apr. 8, 2003 |
| OSMOTIC DELIVERY SYSTEM HAVING | 10/354,142 | 20030139732 | 6,872,201 |
| SPACE EFFICIENT PISTON | Jan. 30, 2003 | Jul. 24, 2003 | Mar. 29, 2005 |
| MINIMALLY COMPLIANT, VOLUME- | 10/606,407 | 20040019345 | 6,939,556 |
| EFFICIENT PISTON FOR OSMOTIC DRUG | Jun. 25, 2003 | Jan. 29, 2004 | Sep. 6, 2005 |
| DELIVERY SYSTEMS | | | |
| OSMOTIC DELIVERY DEVICE HAVING A | 10/302,104 | 20040102762 | 7,014,636 |
| TWO-WAY VALVE AND A DYNAMICALLY | Nov. 21, 2002 | May 27, 2004 | Mar. 21, 2006 |
| SELF-ADJUSTING FLOW CHANNEL | | | |
| SUSTAINED DELIVERY OF AN ACTIVE | 10/645,293 | 20040039376 | 7,655,257 |
| AGENT USING AN IMPLANTABLE SYSTEM | Aug. 20, 2003 | Feb. 26, 2004 | Feb. 2, 2010 |
| OSMOTIC DELIVERY SYSTEMS AND PISTON | 12/658,570 | 20100185184 | 7,879,028 |
| ASSEMBLIES FOR USE THEREIN | Feb. 9, 2010 | Jul. 22, 2010 | Feb. 1, 2011 |

Intarcia's platform technology provides a subcutaneous delivery device. The device is a small, matchstick-sized osmotic pump that delivers medication subcutaneously. Each device contains enough medication to treat a patient for a predetermined period of time. The device is activated when subcutaneous tissue fluid passes through the device inlet, expanding the osmotic engine. The osmotic engine drives the piston at a constant rate, delivering consistent drug levels through the device outlet. The device can be inserted in a subcutaneous space in various locations on the arms and abdomen during an in-office procedure, taking as little as five minutes. Delivering drugs via the device avoids peak drug levels and sub-therapeutic troughs, and the unique formulations maintain stability of proteins and peptides at human body temperature for extended periods of time.

ITCA 650 was a clinical stage candidate by Intarcia for treating type 2 diabetes. It uses the DUROS delivery system to deliver exenatide, an approved incretin mimetic. The DUROS system is a small device made of a cylindrical titanium alloy reservoir that is inserted under the skin. Water from the extracellular fluid diffuses through a semipermeable membrane into a salt osmotic engine that drives a piston at a controlled rate, resulting in slow and consistent release of the drug formulation.

A diagram showing how DUROS' implantable device works is shown in FIG. 2 (prior art).

In the implantable device shown in FIG. 1, there are three specific chambers and therefore four barriers separating these chambers from one another. Let us start by describing the device from the left side and work our way to the right. On the left end of the device there is a semipermeable plug, or more specifically, an osmotic membrane (I). This membrane allows for fluid from the body to flow into the device. The diffusion mechanism is osmosis because in the first chamber (II), there is a highly concentrated salt solution (>1000 nM as the physiological ionic concentration in the human body is about 150 nM) that will result in liquid being drawn into the implant. As liquid is drawn into the chamber, its volume increases, and this pressure pushes the piston (III), which could be made of a magnetic material, between the first and second chambers to the right. The cylinder surrounding the piston (III) is preferably made of a non-magnetic material, such as a non-magnetic metal or a non-magnetic ceramic material. The non-magnetic metal could be titanium, stainless steel, cobalt-chromium, tungsten, tantalum, or a host of metals that are used in cardiovascular, orthopedic, and many other medical device fields, so long as these metals are non-magnetic.

As the piston is pushed to the right by osmotic pressure, it decreases the volume of the middle chamber (IV). This chamber contains the drug in beads in a flowable mixture, which could be an emulsion, and is interchangeably referred to as “emulsions.” The beads could be 10-100 μm in diameter, and have shells made of a material that is a stimuli-responsive polymer, preferably a stimuli-responsive biodegradable polymer, such as those disclosed in U.S. Patent Publication 20060127925, titled “Stimuli-responsive polymer conjugates and related methods,” U.S. Patent Publication 20160263221, titled “MULTI-RESPONSIVE TARGETING DRUG DELIVERY SYSTEMS FOR CONTROLLED-RELEASE PHARMACEUTICAL FORMULATION,” U.S. Patent Publication 20170119785, titled “SOL-GEL POLYMER COMPOSITES AND USES THEREOF,” U.S. Patent Publication 20170165201, titled “PH-RESPONSIVE MUCOADHESIVE POLYMERIC ENCAPSULATED MICROORGANISMS,” U.S. Patent Publication 20170135953, titled “METHODS FOR LOCALIZED DRUG DELIVERY,” U.S. Patent Publication 20170073311, titled “SUPRAMETALLOGELS AND USES THEREOF,” and U.S. Patent Publication 20170065721, titled “SYNTHESIS AND USE OF THERAPEUTIC METAL ION CONTAINING POLYMERIC PARTICLES” which are incorporated, in relevant parts for the purposes of written description, herein, by reference in their entirety. The stimuli-responsive polymer could be a temperature-sensitive polymer, a pH-sensitive polymer, an electrical or magnetic field-sensitive polymer, or a light-sensitive polymer. An example of a stimuli-responsive polymer is poly(N-isopropylacrylamide). A pH-sensitive polymer could dissociate or dissolve at the blood pH of between 7.35 and 7.45. For example, the shell could be made of a polymer held together by a polymer cross linker. The polymer could be a pH-sensitive or a temperature-sensitive polymer that dissociates or dissolves at the blood pH of about 7.4 or the normal human body temperature of 36.5-37.5° C. (97.7-99.5° F.). Thus, the pH of this chamber within the implantable device would be lower than that of blood (below, 6.5, preferably below 5, e.g., between 1-3) or higher than that of blood (above 8, preferably above 10, e.g., between 11-14), preventing any of the drug molecules from exiting through the beads in the acidic or basic flowable mixture (e.g., emulsion) within the implant. Once the beads reach the bloodstream, however, the drug would begin coming out of the beads as the beads dissociate or dissolve in blood. The shell of the bead could also contain an antibody for tumor targeting in the case of cancer as well as gold particles to enhance radiation therapy. These are discussed in more detail in a later section.

As the piston gets pushed to the right, some of the bead-containing emulsion in the second middle chamber flows to the final chamber (VI). This third chamber would contain a foam, e.g., a biodegradable foam, that works as the flow control mechanism. The emulsions would pass through the porous foam before reaching the 100-500 μm openings bordering the circumference of chamber VI. These openings release the bead-containing emulsion from the implantable device to the bloodstream. The foam would act to prevent blood from entering the device and back-filling it. In addition to just acting as an obstruction between the device and the blood, the foam can act to control the flow rate of the emulsions from the device to the body. More specifically, the foam will have the property that it can be made more porous when sonicated. Thus, when the flow rate of the drug into the bloodstream is smaller than desired, sonication can be used to create more porosity in the foam. Another way of creating greater porosity through the foam would be through a controlled explosion within the foam. Thereby, as the foam disintegrates, it is easier for the emulsions to traverse from the middle chamber out into the body.

The open cell biodegradable foam could be reticulated foam formed after thermal reticulation, such as that disclosed in U.S. Pat. No. 8,801,801, titled “AT LEAST PARTIALLY RESORBABLE RETICULATED ELASTOMERIC MATRIX ELEMENTS AND METHODS OF MAKING SAME,” which is incorporated herein, in relevant parts for the purposes of written description, in its entirety. In the biodegradable reticulated foam, the boundary skin layer formed during the foaming process was trimmed and removed prior to subjecting the as-made foam to thermal reticulation. The open cell biodegradable foam in the embodiments herein is generally resilient to crushing when implanted within the body of a subject; thereby the open cell biodegradable foam substantially maintains its original shape before implantation even after implantation within the body of the subject.

It is preferable to be able to control the flow rate of drug release and to have a multi-functional implant. More specifically—assuming that we want a constant output of the pharmaceutical product—as liquids from the body enter the first chamber on the left due to osmosis, the pressure on the piston will diminish over time. Thus, the rate at which drug exits the implantable device will also decrease over time. To solve this problem, the implantable device will have a sensor on or in the rightmost side wall (VII), e.g., an impermeable plug, and the sensor will measure the concentration of drug in the blood at that spot. When the concentration is below the desired threshold, the sensor will indicate to the on-implant sonicator (V) to turn it on for a set number of seconds. This sonicator is the barrier between the second and third chambers or within the third chamber as shown in FIG. 1, and as it sonicates, the foam will degrade, making easier access for the drug-containing emulsions to reach the openings bordering the bloodstream. In this manner, the drug is released with a substantially constant concentration if the concentration of the drug is within limits of Cₘₐₓ and Cₘᵢₙ as shown in FIG. 3.

The limits of Cₘᵢₙ and Cₘₐₓ are 80% to 125% of a desired concentration (which would be between the limits of Cₘᵢₙ and Cₘₐₓ depicted in FIG. 3) of the drug in the body of the subject. These limits of Cₘᵢₙ and Cₘₐₓ are selected in the context of this disclosure because the United States FDA considers two products bioequivalent if the 90% confidence interval of the peak concentration of a drug in the blood serum of a test sample (e.g. generic formulation) to reference (e.g. innovator brand formulation) is within 80% to 125% of a desired concentration of a drug in the blood serum.

The desired drug concentration could be substantially constant with time. The desired concentration could be substantially constant for a first period of time and substantially zero for a second period of time, or vice versa, or any combinations thereof. The desired concentration could be increasing or decreasing with time, or any combinations thereof.

In addition to dissolving or degrading the foam to increase the porosity of the foam and thereby increase the flow rate of the device, it may be necessary to lessen or even completely stop the drug release in certain circumstances. This can be done by applying an external magnet, such as an electromagnet, atop the implantable device to prevent the piston, which could be made of magnetic material, from moving despite osmotic pressure. The strength of the magnetic force would be greater than that of the fluid pressure, thereby stalling the piston. Since the implantable device will be inserted either in the arm or near the stomach, such a magnet could be applied on an armband or belt surrounding the device. Such a capability would allow a patient to temporarily stop drug release completely. Also, in case it is difficult to sonicate the biodegradable foam internally, a sonicator can be applied externally on such an armband or belt. In this case, the biosensor for drug concentration could send a signal to the patient (perhaps a text message to a phone or an email to a computer) indicating that the patient must apply the sonicator.

Further, instead of or in addition to having a foam for controlled release of the emulsions, the implantable device could encompass a plate in lieu of or in addition to the sonicator (both are shown with numeral V in FIG. 1), as shown in FIG. 4, containing holes, some or all of which are filled with phase-change material (PCM). Similar to the physical (sonification) dependence of the state of the foam and the chemical (pH) dependence of the state of the emulsions, the PCM is temperature dependent. Thus, when the sensor indicates that there is not enough drug in the bloodstream, the patient could add heat to the implantable device via the external belt or armband in order to melt the PCM and unclog the holes. As these holes are opened up, the bead-containing emulsion from the middle unit will flow more easily into the third chamber and eventually leave the device. Hence, this would be an alternative rate control mechanism.

In another embodiment, the first chamber containing the salt solution and the second chamber containing the bead-containing emulsion can be recharged, for example, using a syringe containing the salt solution or the bead-containing emulsion, even when the implantable device is implanted in the body of the subject without removing the implantable device from the body. This could be done through one or more hermetically sealed valves in the casing, with the valves located in positions above the first and second chambers. The hermetically sealed valve could be a pneumatically sealed valve or a slit valve such as those disclosed in U. S. Patent Publication 20140142556, titled “Implantable Drug Delivery Devices” which is incorporated, in relevant parts for the purposes of written description, herein by reference in its entirety.

One way to refresh and/or increase the osmotic pressure of the salt solution in the first chamber after implantation of the implantable device in the body would be to extract the used salt solution from the first chamber and replace it with a fresh salt solution. Similarly, one way to refresh and/or increase the concentration of the drug in the second chamber after implantation of the implantable device in the body would be to extract the used emulsion containing the drug-containing beads from the second chamber and replace it with a fresh emulsion containing the drug-containing beads or add a fresh emulsion containing the drug-containing beads without removing the existing emulsion from the second chamber.

In another embodiment, the emulsion containing the drug-containing beads could contain multiple drugs (a cocktail of drugs). In this case, different drugs could be encapsulated in different types of beads, with the different types of beads having a certain affinity for binding to different organs or tissues of the body.

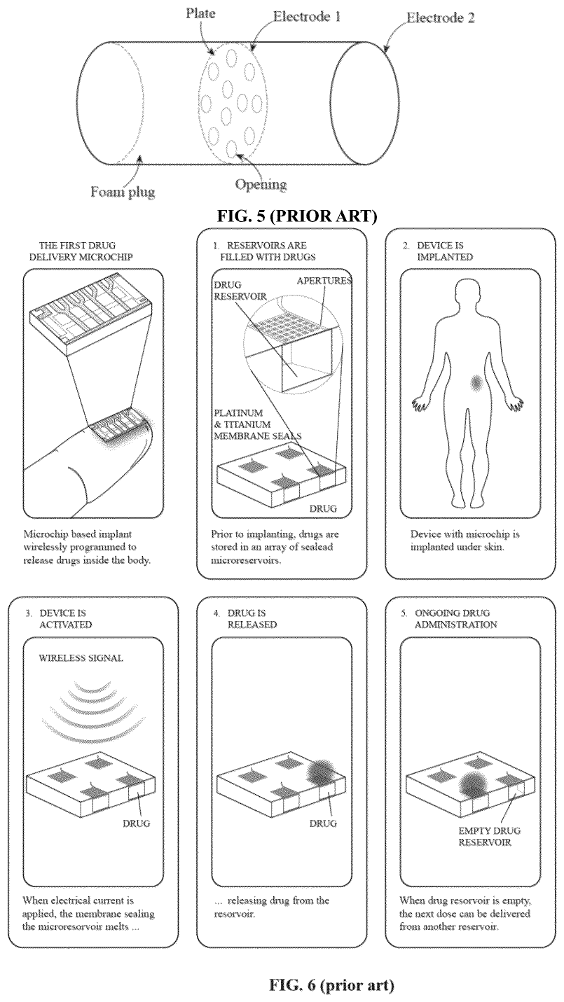

FIG. 5 shows a foam plug with a plate, rotated view of plate in FIG. 4, located within the plug. The plate could also function as an electrode (electrode 1) with a counter electrode (electrode 2) located around one end of the foam plug. The combination of electrodes 1 and electrode 2 could be used for creating holes in the plate of FIG. 4 or to enhance or slow down the flow rate by electrophoresis, which causes the motion of dispersed particles, relative to a fluid under the influence, of a spatially uniform electric field.

Also, instead of applying external heat, the implantable device could internally contain electrodes that heat the PCM plate and dissolve some of the clogged holes. Different-sized holes could be plugged with PCMs of different melting points to ensure greater flexibility in controlling flow rate. MicroCHIPS® technology, which was originally created in the 1990s by MIT researchers Robert Langer and Michael Cima and PhD student John Santini, who later licensed it out to MicroCHIPS®. A diagram showing how MicroCHIPS® wireless implantable device works is shown in FIG. 6 (prior art). The following are the US patents of Langer and Cima, and these US patents are incorporated herein, in relevant parts for the purposes of written description, in their entirety by reference.
U.S. Pat. No. 8,403,907 Full-Text Method for wirelessly monitoring implanted medical device.
U.S. Pat. No. 8,308,707 Full-Text Method and system for drug delivery to the eye.
U.S. Pat. No. 7,918,842 Full-Text Medical device with controlled reservoir opening.
U.S. Pat. No. 7,901,397 Full-Text Method for operating microchip reservoir device.
U.S. Pat. No. 7,892,221 Full-Text Method of controlled drug delivery from implantable device.
U.S. Pat. No. 7,879,019 Full-Text Method of opening reservoir of containment device.
U.S. Pat. No. 7,776,024 Full-Text Method of actuating implanted medical device.
U.S. Pat. No. 7,354,597 Full-Text Microscale lyophilization and drying methods for the stabilization of molecules.
U.S. Pat. No. 7,226,442 Full-Text Microchip reservoir devices using wireless transmission of power and data.
U.S. Pat. No. 7,070,592 Full-Text Medical device with array of electrode-containing reservoirs.
U.S. Pat. No. 7,070,590 Full-Text Microchip implants.
U.S. Pat. No. 6,976,982 Full-Text Flexible microchip devices for ophthalmic and other applications.
U.S. Pat. No. 6,808,522 Full-Text Microchip devices for delivery of molecules and methods of fabrication thereof.
U.S. Pat. No. 6,537,256 Full-Text Microfabricated devices for the delivery of molecules into a carrier fluid.
U.S. Pat. No. 6,491,666 Full-Text Microfabricated devices for the delivery of molecules into a carrier fluid.
U.S. Pat. No. 6,123,861 Full-Text Fabrication of microchip implants.
U.S. Pat. No. 5,797,898 Full-Text Microchip implants.
U.S. Pat. No. 5,514,378 Full-Text Biocompatible polymer membranes and methods of preparation of three dimensional membrane structures.

Similar technology as that disclosed in the patents of Langer and Cima can also be employed to generate controlled explosions of PCMs by generating electrical current and burning or melting the PCM membrane sealing the holes, thereby unplugging the holes. The explosions in the plate with PCM in holes can be done similar to that in the MicroCHIPS® implant, for example, as and when required using a controller and appropriately spaced-apart electrodes, either directly and automatically based on feedback from the sensor or externally by a person using a remote-control device. Also, the explosions can be programmed ahead of time into the implantable device when there is a reliably tested dosage cycle.

Furthermore, some of the openings bordering the circumference of chamber VI of FIG. 1 can also be filled with phase-change material (PCM). These openings could be explosively opened as explained above in the context of opening the holes in the plate V of FIG. 4 wherein the holes are filled with phase-change material (PCM).

Besides the sensor being attached to the implant, there could be a secondary sensor inserted elsewhere in the body, preferably near a target site. The secondary sensor could be a biodegradable sensor, for example, such as that developed by a team from University of Illinois in Urbana and Washington University in St. Louis and published in the Jan. 18, 2016, issue in the journal Nature, and subsequently disclosed on Jan. 26, 2017, in U.S. Patent Publication 20170020402, titled “Implantable And Bioresorbable Sensors,” which is incorporated herein, in relevant parts for the purposes of written description, in its entirety by reference.

Furthermore, the micro or nano beads could be of two or more types—those that rapidly break-up as soon as they leave the implantable device and those that break-up after a longer period (several days) after exiting the implantable device and traveling to the target site. The shell of the rapidly disintegrating beads could contain starch or cellulose.

In one embodiment, it is not necessary to include the first chamber and have an osmosis-driven piston. Instead, the piston could be driven electro-magnetically using electromagnets in a device external to the body in which the implantable device is implanted. In this case, the external device could be manufactured with circuitry that controls the movement of the piston. This system would allow extremely precise flow rates for the emulsions based on feedback from the sensor attached to the implantable device or the secondary sensor inserted elsewhere in the body, preferably near a target site. The sensors could send signals indicating when to push, pull, or stop the piston entirely to allow for pre-programmed drug release for the patient.

The implantable device of the embodiments herein may require an energy source that is biocompatible and produces electricity. The energy source could be a battery, photo-energy source, or a galvanic cell. The batteries in the implantable device of the embodiments could be charged externally by radiofrequency (RF) charging.

In terms of target specificity for the beads, especially in the case of cancer, it is important to lead the drug-encapsulated beads to the tumor site. Thus, each bead will contain an antibody, corresponding to antigens on the site of the tumor, on its surface. The antibody would be joined by a linker to the shell of the bead and would ensure that the drug is attracted toward the tumor as opposed to just free-floating in the blood. If the site of the tumor is known in advance, an additional sensor could also be placed there to measure drug concentration. This sensor can communicate with the sensor on the implantable device and thereby request for greater drug release in case the tumor is not being properly attacked. This interplay between sensors would greatly define the conditions for controlled drug release. In addition, these beads could have gold or platinum (or any other inert metal) attached to the surface of the beads, e.g., composite inorganic organic nanoclusters (COINs), to tremendously help patients undergoing radiotherapy. For example, U.S. Patent Publication 20160129111, titled “Methods For Delivering An Anti-Cancer Agent To A Tumor,” which is incorporated herein, in relevant parts for the purposes of written description, herein by reference in its entirety, discloses methods for delivering an anti-cancer agent to a tumor in a subject. The method involves administering to the subject (i) gold particles and (ii) at least one-anti-cancer agent directly or indirectly bonded to the macromolecule and/or unbound to the macromolecule and exposing the tumor to light for a sufficient time and wavelength in order for the gold particles to achieve surface plasmon resonance and to heat the tumor.

COINs are composed of a metal, preferably an inert metal such as gold or platinum, and at least one organic radiation-active compound. For example, Raman-active COINS are disclosed in U.S. Pat. No. 7,790,286, which is incorporated herein, in relevant parts for the purposes of written description, in its entirety by reference. Interactions between the metal of the clusters and the radiation-active compound(s) enhance the radiation signal obtained from a radiation-active compound when the nanoparticle is excited. Since a large variety of organic radiation-active compounds can be incorporated into the nanoclusters, a set of COINs can be created in which each member of the set has a radiation enhancement unique to the set. Also, COINs can also function as sensitive reporters for highly parallel detection of the beads. Furthermore, treatment specificity can be enhanced by incorporating thousands of gold or platinum particles into a single nanocluster and/or attaching multiple nanoclusters to a single bead.

More specifically, once the beads are attached to the tumor site, the gold or platinum particles amplify the radiation that is presented at the tumor site, preventing the need for exorbitantly high levels of radiation that can oftentimes be dangerous to the individual. Thus, through the combination of a target antibody, a sensor at the tumor site, and gold/platinum particle technology, the implantable device would address the issue of treatment specificity, which is incredibly problematic in oncology care.

In one embodiment, the nano or micro beads could comprise a shell comprising a first material and a second material, wherein the second material comprises a biodegradable material; a core comprising a pharmaceutically effective composition, the core being enclosed by the shell; wherein the first material is distributed in the biodegradable material; wherein the first material is configured to create holes in the shell; wherein the holes allow the pharmaceutically effective composition to be released to the exterior of the shell through the holes. In one embodiment, the shell could be made by polymerizing a silica-functionalized monomer to form a silica-containing biodegradable polymer shell.

In the implant, the pharmaceutically effective composition could comprise a targeting material or molecule that binds to a certain organ, object, or a specific site within a body of a subject. Thus, for example, even if the drug is the same but used for different cancers, a targeting molecule for a particular type of cancer would bind to the cells of that particular type of cancer. On the other hand, if the drug is intended for ovarian cancer, then the target molecule could be specifically one that binds to the cells of ovarian cancer. The targeting material or molecule could be a biomarker.

An ingredient of the pharmaceutically effective composition could be a material that prevents the pharmaceutically effective composition from being taken up by the host defense as white blood and macrophages in the subject's body. Such an ingredient remains in the blood but the body organs cannot take it up. An example of such an ingredient is polyethylene glycol (PEG), e.g., having 200 Dalton molecular weight.

In another embodiment, the biodegradable shell of the nano or micro beads could contain a controlled release ingredient that functions as a control release sensor to control the release of the drug. For example, one would want a particular concentration of the drug at a given site over a given extended period of time. The control release ingredient could be a material that degrades faster than the remaining material of the biodegradable shell of the nano or micro beads or punches holes (openings) in the biodegradable shell of the nano or micro beads.

The holes in the biodegradable shell of the nano or micro beads could be punched by giving external stimuli such as sound, such as ultrasonic sound, radio frequency heating, radiation or microwave to the drug containing beads. The external stimulus heats up the controlled release ingredient, thereby punching one or more holes in the shell surrounding the controlled release ingredient, which could be a metal-containing material to form holes in the shell, before or after implanting or attaching the device in or on a body of a subject, when the shell is exposed to an external stimulus. For example, the controlled release ingredient that can be used for punching holes in the shell could be molecular iron such as a magnetic resonance imaging (MRI) contrast agent. Depending on the concentration of the controlled release ingredient, one can control the number of holes punched in the shell, which in turn controls the amount of drug released from the core to the outside of the shell.

Two types of iron oxide MRI contrast agents exist: superparamagnetic iron oxide (SPIO) and ultrasmall superparamagnetic iron oxide (USPIO). These contrast agents consist of suspended colloids of iron oxide nanoparticles. An FDA approved iron oxide MRI contrast is Lumirem (also known as Gastromark).

Other controlled release ingredients for punching holes in the shell could be superparamagnetic iron platinum particles (SIPPs). SIPPs could also be encapsulated with phospholipids to create multifunctional SIPP stealth immunomicelles that specifically target human prostate cancer cells.

Yet, other controlled release ingredients for punching holes in the shell are Mn-based nanoparticles. Manganese ions (Mn²⁺) are often used as a contrast agent in animal studies, usually referred to as MEMRI (Manganese Enhanced MRI). For example, Mn²⁺ carbon nanostructure complexes of graphene oxide nanoplatelets and graphene oxide nanoribbons could also be used as controlled release ingredients.

In addition to or in lieu of the osmotic pump option of pumping the emulsion from the pump chamber, a shape memory alloy (e.g., nitinol) spring could be connected to the piston and a hook inside the implantable device opposite the piston could be employed. For example, the hook could be attached to the plate V shown in FIG. 4 and discussed above, or to the front end of the implantable device attached to the wall on the right side of the third chamber (IV) in FIG. 1. The shape memory alloy spring could be heated using external heating, e.g., radio frequency heating.

The advantages of the present disclosure will become readily apparent by those skilled in the art from the following detailed description, wherein it is shown and described in the embodiments, simply by way of illustration of the best mode contemplated for carrying out the disclosed methods. As will be realized, the device, system and method disclosed is capable of other and different embodiments, and several details are capable of modifications in various obvious respects, without departing from the disclosure. Accordingly, the description is to be regarded as illustrative in nature and not as restrictive.

### Uses Thereof

Use of such device is for systemic delivery of any active pharmaceutical ingredient in a subject, as a patient convenience to avoid oral, subcutaneous, and intravenous administration on a daily, weekly, or monthly basis.

Use of such device is for systemic delivery of any biologic therapy in a subject as a means of patient convenience to overcome daily, weekly, or monthly oral, subcutaneous, or monthly administration. Biologic molecules can be peptides, antibodies, or fragments thereof, nucleic acid molecules such as modified RNA, small interfering RNA, anti-sense DNA molecules or fragments thereof.

Use of such a device to deliver antigens to elicit vaccine responses in a subject.

Use of such a device to provide local tissue delivery of any therapeutic molecule, be it small molecule or biologic.

Use of such a device to provide long-term sustained release of insulin or analogs thereof, alone or in combination with other therapies, to treat diabetes or other metabolic conditions.

Use of such a device to provide long-term sustained release of GLP-1 or analogs thereof, alone or in combination with other therapies, to treat diabetes or other metabolic conditions.

Deposition of a such device underneath the skin, or in fat tissue or in any specific organ for the purpose of long-term release of any therapeutic molecule, be it either a small molecule or biologic.

Specific use of such a device to provide sustained long-term release of contraceptive hormones, combination of estrogen or progestin or singular delivery of progesterone alone to serve as contraceptive aid for women.

Specific use of such device for the intraocular delivery of any small molecule or biologic therapy as a means to provide the long-term therapeutic benefit for ocular diseases, such as age-related macular degeneration, dry eye, and various others. Deposition of such device into bladder for long-term delivery of any small molecule or biologic therapy to treat urinary bladder complications such as incontinence, yeast infections, bladder cancer and various others.

Deposition of such a device to locally deliver therapeutic molecules to the male reproductive organs as a means to treat medical conditions such as erectile dysfunction, premature ejaculation, testicular cancer, and various others pertaining to male reproductive system.

Deposition of such device and use to locally deliver therapeutic molecules to female reproductive organs, uterus, and ovaries, to treat medical conditions such as endometriosis, uterine fibroids, ovarian cancer, uterine cancer, poly cystic ovarian syndrome, and various other disease pertaining to female reproductive system.

Deposition of such device and use to locally deliver therapeutic molecules to heart conditions such as heart failure, myocardial ischemia, and various other heart diseases.

Deposition of such device and use to locally deliver therapeutic molecules into the adipose tissue to treat conditions such as metabolic syndrome, diabetes, hypercholesterolemia, hypertriglyceridemia, and various others.

In embodiments herein, depending on the dosing requirements of a particular drug, the device can provide a constant drug concentration or even an increasing drug concentration or a decreasing drug concentration over time. The device can also provide a constant concentration for a predefined time, followed by no drug release for some time, and subsequently followed by the same concentration from the beginning. This would be especially useful in the case of birth control medication; for a short period of time when a woman wants to get pregnant, the implantable device can stop the release of the drug, and then continue it again afterwards. Thus, embodiments herein relate to a highly controlled delivery system by which any permutation of drug release profiles could be either programmed ahead of time or even implemented in real-time.

### E. Osmotic Pump-Based Drug Delivery System

The membrane plug functions to seal the interior of the implantable device from the external environment, allowing only specific liquid molecules to permeate through the membrane plug and into the device's interior. The membrane plug effectively prevents items within the implantable device, including the osmotic agent and the drug, from passing through it. The rate at which liquid passes through the membrane plug and enters the device is dependent on factors such as the membrane material type, as well as the size and shape of the membrane plug.

By controlling the rate at which liquid passes through the membrane plug, the expansion of the osmotic agent is also controlled, driving the drug through the valve to the delivery orifice. Therefore, the delivery rate of the drug from the implantable device can be regulated by adjusting either the permeability coefficient or size of the membrane plug.

After the implantable device is introduced into the subject's body, body fluid penetrates the semi-permeable membrane and interacts with the osmotic agent, resulting in its expansion and exertion of osmotic pressure on the drug delivery unit. The osmotic pressure subsequently causes the release of the drug formulation through the delivery orifice once the valve is in an open state.

Theoretically, the liquid permeation rate dV/dt through a semipermeable membrane included in an osmotic pump of the present invention is equal to the liquid permeability coefficient P for the membrane forming material multiplied by the exposed surface area of the membrane A and the osmotic pressure difference Δπ generated between the interior of the reservoir and the internal body part of the subject by the osmotic agent, divided by the thickness of the membrane sheet L.

$$dv / dt = PA Δ π / L$$

When the valve is in an open state, the drug delivery rate dMt/dt is theoretically equal to the liquid permeation rate dV/dt multiplied by the concentration C of the drug.

$$dMt / dt = dv / dt . C = \{ PA Δ π / L \} . C$$

Therefore, even where Δπ and C remain the same, the drug delivery rate provided by the osmotic pump of the present invention can be increased by increasing A (the amount of surface area of the membrane exposed), decreasing L (effective thickness of the membrane), or increasing P (the liquid permeability coefficient of the membrane forming material), or any of those combinations.

In an embodiment, the implantable osmotic pumps have been typically designed to provide substantially zero-order release rates of a desired therapeutic agent. In some embodiments, the semipermeable membrane has a substantially constant permeability wherein the permeability rate is independent of concentration difference across the semipermeable membrane.

In an embodiment, the implantable osmotic pumps have been typically designed to provide pseudo zero-order flux (flow) of the solvent through the semipermeable membrane.

In an embodiment, a constant flux of the solvent in the osmotic unit is maintained by varying the permeability of the semipermeable membrane such as increasing the permeability of the semipermeable membrane with the decrease in the difference of the concentration across the semipermeable membrane.

In an embodiment, the osmotic membrane may contain a metal particle, particles, or metal strip that when exposed to a strong external magnetic field tears or pulls apart the osmotic membrane. In an emergency or other situation where the device dosing needs to be halted, a strong magnetic field may be externally applied to the device (e.g., A strong magnet external to the patient's skin) causing the osmotic membrane to no longer function effectively, thus stopping the dosing.

Materials Suitable for use in formulating the semipermeable material included in an osmotic pump according to the present invention are taught, for example, in U.S. Pat. Nos. 4,874,388, 5,234,693, 5,279,608, 5,336,057, 5,728,396, 5,985,305, 5,997,527, 5,997,902, 6,113,938, 6,132,420, 6,217,906, 6,261,584, 6,270,787, 6,287,295, and 6,375,978, the contents of each of which are incorporated herein in their entirety by reference. Suitable materials include, but are not limited to, polyolefins including polyethylene, polyvinyl acetate and ethylene vinyl acetate copolymers, Hytrel polyester elastomers (DuPont), cellulose esters, cellulose ethers and cellulose ester-ethers, water flux enhanced ethylene-vinyl acetate copolymers, semipermeable membranes made by blending a rigid polymer with water-soluble low molecular weight compounds, and other semipermeable materials well known in the art. High density polyethylene and ethylene vinyl acetate copolymers represent preferred rate controlling membrane materials according to the present invention.

In one embodiment of the osmotic pump of the present invention, the semi permeable membrane itself is designed or formulated to provide a membrane that exhibits a permeability that increases as the osmotic pump functions in an environment of operation. In another embodiment, the osmotic pump of the present invention includes a semi permeable membrane that exhibits a substantially constant permeability but is designed such that the surface area of the semi permeable membrane exposed to the environment of operation increases automatically as the osmotic pump functions. In yet another embodiment, the osmotic pump of the present invention includes a semi permeable membrane designed or formulated to exhibit a permeability that increases and is designed such that the surface area of the semipermeable membrane exposed to the environment of operation increases automatically as the osmotic pump functions. In a further embodiment, the osmotic pump of the present invention includes a semi permeable membrane that exhibits a substantially constant permeability but is designed such that the effective thickness of the semi permeable membrane can be decreased to, in turn, increase the release rate of the osmotic pump. In yet another embodiment, the osmotic pump of the present invention includes a semi permeable membrane designed or formulated to exhibit a permeability that increases and is designed such that the effective thickness of the semi permeable membrane can be decreased to, in turn, increase the release rate of the osmotic pump.

1. Osmotic Pump-Based Drug Delivery System with Non-Fixed Pressure and Controlled Valve

Another embodiment relates to a device comprising a first chamber comprising a drug reservoir unit and an osmotic unit; a second chamber comprising an electronic unit; a third chamber comprising a drug delivery unit; wherein the first chamber and the third chamber are fluidically connected via a valve; and wherein an opening and closing of the valve is controlled via the electronic unit; wherein the device is configured to deliver a drug into a body of a subject; wherein the device is implantable into the body of the subject; and wherein the device is configured to mimic a flow pattern of repeated injections to deliver a constant volume of the drug during each injection of the repeated injections.

Valve may have an orifice or other flow restriction to regulate the flow while the valve is open. With a great enough restriction, the valve may be open for a substantial time to provide the desired dose. This allows the system to provide a near continuous dosing method if desired.

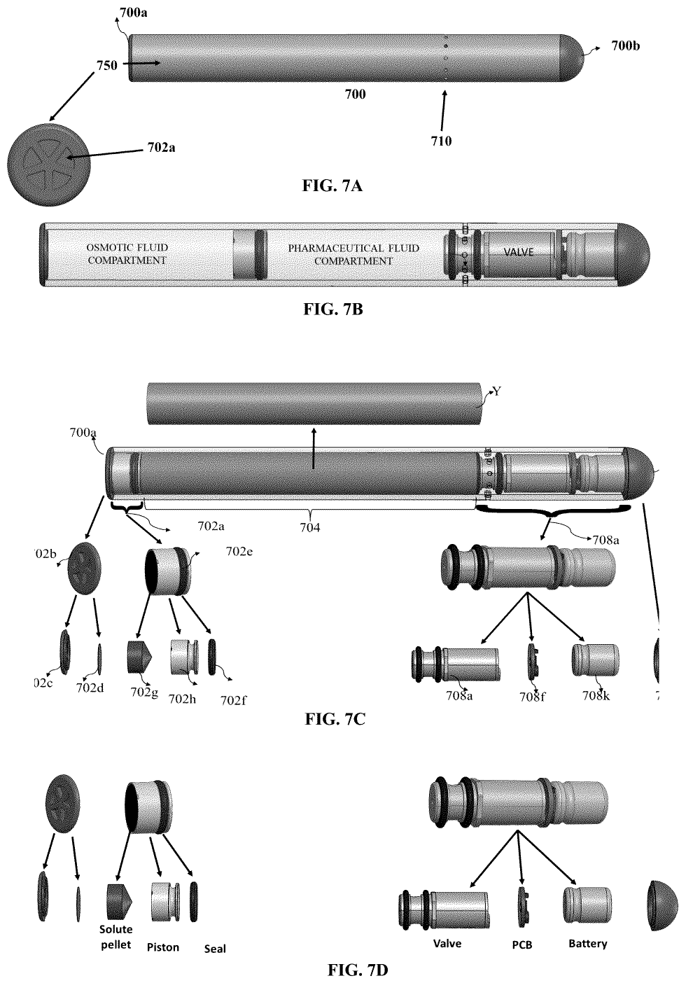

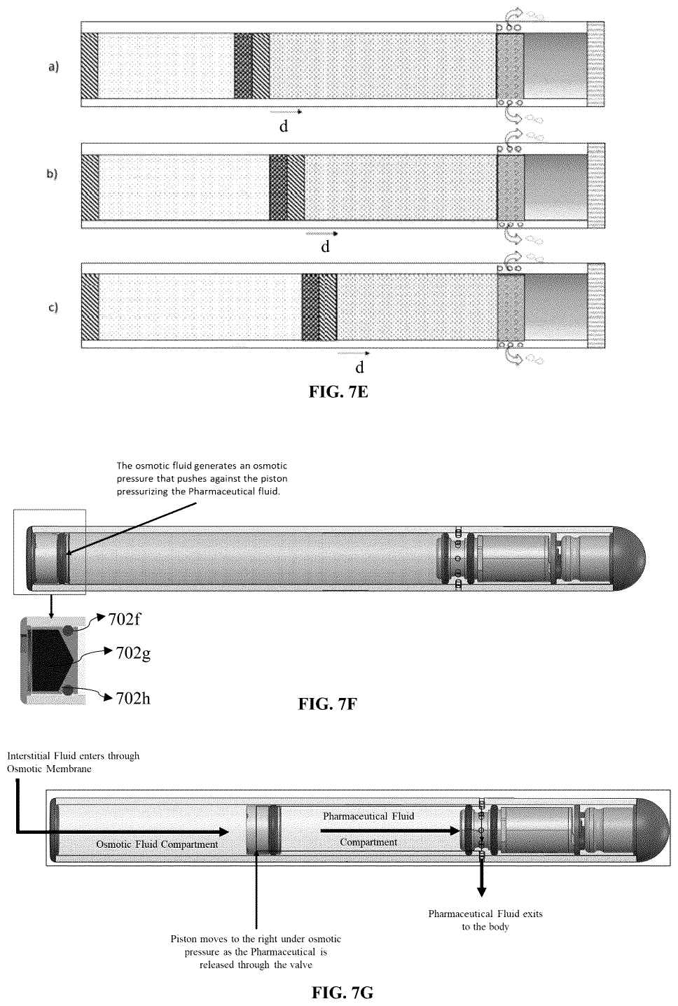

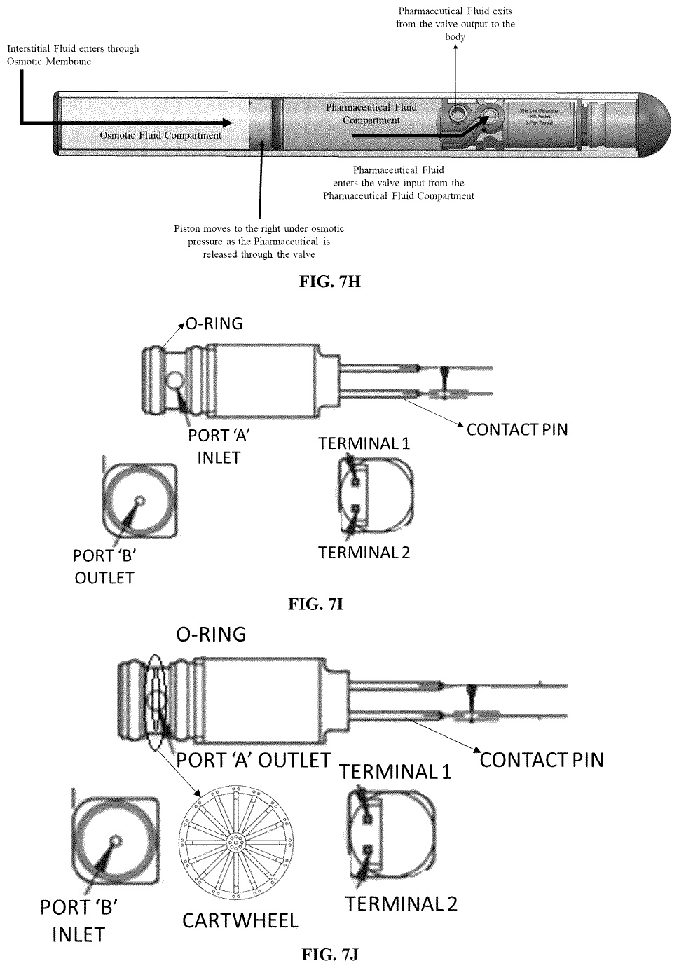

Referring to FIG. 7A, it shows an exterior view of the device, according to one or more embodiments. The device comprises a cylindrical body 700 comprising a first end 700a, a second end 700b and a plurality of orifice 710 radially distributed across the circumference of a drug delivery unit 706 (not shown in this figure) encased in the cylindrical body 700. The first end 700a comprises a membrane plug 702a that allows interstitial body fluid 750 to flow inside the cylindrical body 700. Additionally, the orifices may be one or more slots or other shaped openings.

Referring to FIG. 7B, it shows an exterior view of the device, according to one or more embodiments. The device comprises a membrane cap at one end adjacent to an osmotic fluid compartment and an end cap adjacent to the electronics. The device comprises an osmotic fluid compartment, a piston assembly, a pharmaceutical compartment, a drug delivery compartment, a valve and an electronic assembly compartment. The osmotic fluid compartment comprises an osmotic agent that can exert an osmotic pressure to the piston assembly. The pharmaceutical compartment comprises a drug formulation (described in Section C of this specification). The drug delivery compartment comprises a plurality of holes distributed radially across the circumference of the implantable device. The electronic assembly compartment comprises the electronics of the implantable device. The valve can be actuated and controlled by the electronics and is configured to control a delivery of the drug formulation into the body portion on a subject where it is implanted.

Referring to FIG. 7C, it shows an exploded view of the interior of the device according to one or more embodiments. The first end 700a of the device comprises an osmotic assembly 702a. The osmotic assembly 702a comprises a membrane plug 702b and a piston assembly 702e. The osmotic assembly 702b is not movable but fixed at the first end 700a. The piston assembly 702e is movable and movement is based on the osmotic pressure exerted by an osmotic unit 702 which is created between the membrane plug 702b and the piston assembly 702e due to inflow of the interstitial body fluid into the device via the osmotic assembly 702b. The osmotic assembly 702b comprises a first end cap 702c and an osmotic membrane 702d. The osmotic membrane 702d allows selective inflow of the interstitial body fluid into the device. In some embodiments, the osmotic membrane 702d is permanently attached to the first endcap 702c. In some embodiments, the first endcap's 702c orifice sizes are primarily determined by maximum osmotic pressure needed and a strength of the osmotic membrane 702d. The piston assembly 702e comprises a piston 702h and a first seal 702f. The piston 702f further comprises a solute 702g inside of a piston hollow opposite to direction of movement of the piston assembly 702e.

Before the implantation of the device, the piston 702f is at the first end 700a, up against the osmotic assembly 702b and the size of osmotic unit 702 at this point is zero. The solute 702g is in a liquid, gel, a pellet, or a powder state. The pellet size (and hollow size) is determined by desired osmotic pressure. Shortly after implantation of the device into the body of a subject, there is a selective inflow of the interstitial fluid in the device (for now on referred to as solvent). At this point, the solute 702g (e.g., salt pellet) gets dissolved via the piston hollow 702h with the solvent. Solute type and concentration can be chosen to provide an osmotic pressure from 1 to 28000PSI.

As soon as the device is implanted and the interstitial fluid starts flowing inside the device, the piston starts moving in the piston assembly 702e, the size of osmotic unit 702 starts to increase. Simultaneously, the solute starts dissolving into the fluid inside the device, creating an osmotic pressure to the backside of the piston assembly 702e. The device comprises a drug reservoir unit 704 that further comprises a drug Y.

The device further comprises an electronic unit 708 that further comprises a valve 708a, a printed circuit board (PCB) 708f, and a battery 708k. PCB 708f controls the valve opening and battery 708k provides power to the device. In some embodiments, electronics also comprise an electromechanical assembly.

The second end 700b of the device is sealed using an end cap 708z. The solute 702g dissolves completely into the solute before the piston assembly 702e reaches towards the valve 708a. The position of the piston assembly 702e, which is between the drug reservoir unit 704 and the osmotic unit 702, is determined by how much drug Y has been discharged into the subject's body. The electronic unit 708 can further comprise a microprocessor that can calculate a time-period for opening and/or closing of the valve 708a and a micro controller that controls the opening and closing of the valve 708a. In some embodiments, the microprocessor and/or the microcontroller are sandwiched between the valve 708a and the battery 708k. In some embodiments, the microprocessor and/or the microcontroller are separated from the PCB 708f but connected through wires.

In some embodiments, the microprocessor or the microcontroller can calculate a corresponding pressure O¹ and O² inside the device for a P¹ piston position (near to the first end) and a P² piston position (near to the second end 700 b meaning very little drug is left in the drug reservoir). Where O² is less than a set minimum pressure, the valve 708a is in an open state for a longer time and when the O² is more than a set maximum pressure the valve 708a is in open state for a shorter time.

Referring to FIG. 7D, it shows sub-assemblies of the implantable device according to one or more embodiments. The Osmotic Membrane is permanently attached to the endcap. The Endcap orifice sizes are primarily determined by maximum osmotic pressure and strength of the membrane. The solute pellet (e.g., salt pellet) is dissolved from the piston hollow shortly after injection into the body. Pellet size (and hollow size) is determined by desired osmotic pressure. In some embodiments, the seal is made of PTFE. The Electronics/Electromechanical Assembly remains fixed in the tubing. The PCB controls the valve opening times. Power is provided by the battery. The valve may be any fluid valve including latching solenoid, NC solenoid, rotary shutter, NC piezo electric, diaphragm valves, etc. (example valves described in Section K of this specification). In an embodiment, the electronic unit 708 does not move within the device. The only moving part is the piston assembly that is sealed for outflow of any fluid.

In an embodiment, the valve is a solenoid latching valve. The solenoid valve is “latched” magnetically in the open position to save energy. Similarly, the solenoid valve is kept in the closed position mechanically through the force of a spring, the operation of which will be described herein below. The solenoid latching valve employs an electromagnetic coil having first and second leads. The solenoid latching valve is controlled by a switch engaging the first and second leads with a direct current voltage source. Alternatively, a dual coil latching valve may be used. One coil is activated for latching open the valve. The second coil is activated for latching the valve closed.

Referring to FIG. 7E, it shows a consistent amount of drug delivery per shot by the implantable device, according to one or more embodiments. The osmotic pump, the valve and the microcontroller work in synchrony, delivering consistent drug levels through the drug delivery orifice. In some embodiments, microcontroller is programmed to open and close the valve depending on real-time osmotic pressure, to deliver a prescribed volume of the drug. In some embodiments, a piston position determination module determines piston position. The valve remains in an open state till the piston moves to a distance d and then it closes. This allows only a constant volume of drug to be delivered in each shot.

In an embodiment, variability per shot of a drug by the implantable device preferably could be ±15% by volume. In some embodiments, per shot variability of a drug by the implantable device, more preferably could be ±10% by volume. In some embodiments, variability per shot of a drug by the implantable device most preferably could be ±5% by volume.

Referring to FIG. 7F, it shows movement of a piston driven by osmotic pressure, according to one or more embodiments. The piston has a hollow cavity that contains the solute pellet (dark blue in cross sectional view). Until insertion into the body, the pellet is dry. After insertion, interstitial fluid enters through the Osmotic Membrane and the pellet starts to dissolve creating an osmotic fluid. The osmotic fluid generates an osmotic pressure that pushes against the piston pressurizing the pharmaceutical fluid. When the valve is opened, the pharmaceutical fluid is forced through the valve by the piston and exits into the body through the radial holes. The valve is held open until the desired dose is achieved and the osmotic fluid rebuilds the pressure. As the piston moves further right with each dose, the osmotic pressure slowly reduces as the solute concentration falls. The valve is kept open longer each dose to account for this pressure drop. A piston position sensor may be used to provide feedback as to dosing size.

In another embodiment, the valve is a non-latching valve. In yet another embodiment, the valve is a piezoelectric valve. In yet another embodiment, the valve is a solenoid valve. In yet another embodiment, the valve is a rotary shutters valve. In yet another embodiment, the valve is a diaphragm valve. In yet another embodiment, the valve comprises a hole that allows flow of the drug from the drug reservoir unit to the drug delivery unit when the valve is in an open state. In yet another embodiment, the volume of drug that is delivered to the subject depends on the time period for which the valve is open. In yet another embodiment, the valve does not move within the device. When the pressure inside the device falls below a defined threshold pressure due to opening of the valve, the microprocessor closes the valve, reducing further drop of pressure. Simultaneously, a predefined amount of solute can be released via the chip when the osmotic pressure inside the device reduces beyond a set threshold pressure.

Referring to FIG. 7G, it shows a schematic of fluid flow in the device according to one or more embodiments. The interstitial fluid (solvent) enters through the osmotic membrane from the first end to the osmotic fluid compartment. The osmotic solution created due to dissolving of the solute with the solvent creates an osmotic pressure on the piston and it moves in the opposite direction of solute inflow, towards the drug reservoir unit. This creates a pressure on the drug reservoir unit and when the valve opens, the drug moves via an opening in the valve to a fluid chamber in the valve. As the valve closes, the fluid flows out to the drug delivery unit which is fluidically connected with the body fluids of the subject's body. This allows the drug to get mixed into the body fluid to perform pharmaceutical action.

Referring to FIG. 7H, it shows a schematic of fluid flow through the valve in the device, according to one or more embodiments. In some embodiments, the valve comprises a fluid chamber that is filled with drugs. The valve comprises a small hole towards the drug reservoir chamber that allows the drug to flow to the fluid chamber of the valve. When a displacement is created within the valve the drug squeezes out to the delivery chamber. Depending on the time period of displacement of the piston pump (also referred to as osmotic pump) from a D⁰ position, a displaced state of the valve determines the amount of drug to be delivered in the subject's body.

In an embodiment, the inlet and outlet of the valve are reversible. In an example the valve may comprise one port as an inlet port and many ports as outlet port. This valve can be used in the opposite way where many ports can act as inlet port and one port can act as outlet port. In some embodiments, the inlet port can be either from the front or from the side of the valve.

Referring to FIG. 7I and FIG. 7J, they show a solenoid valve with non-reversed and reversed inlet port and outlet ports. The valve may comprise a cartwheel structure to distribute one port outlet to many outlets on the periphery of the device. The force on the left side of the plunger of the valve due to the initial osmotic pressure may be less than the force on the right side of the plunger. The drug, from the drug reservoir unit, can enter from the side hole by removing a front O-ring of the valve and connecting a tube from the front outlet of the valve to outlets on the wall of the implantable device.

In an embodiment, the fluid connector is a twin wall disk with fluid transport holes through it. There is a small gap between the two walls of the wall disk that coincides with the holes in the wall of the implantable device. There can be a short tube on the right wall of the wall disk that can tightly fit into the outlet in the valve. As the two-wall circular plate is firmly attached to the left side of the valve, this plate will also provide support to the valve in lieu of the left O-ring.

In another embodiment, the tube cross section may be other than circular including square, rectangular, or oval.

In an embodiment, the valve comprises a wheel shaped distributor. The center outlet of the valve could be shaped like a wheel wherein the spokes are tubes and there is channel around the circumference of the wheel. The center of the wheel can be attached to the outlet of the valve using a tube. The outlet holes in the wall of the implantable device should lie within the channel of the wheel. The combination of the valve and wheel shaped distributor at the center outlet of the valve can allow the valve to operate under higher osmotic pressures.

In some embodiments, the piston assembly may also comprise a chip comprising a phase change material and a solute reservoir. The chip is configured to release a pre-defined amount of solute from the solute reservoir, when an osmotic pressure inside the device reduces beyond a set threshold pressure.

### F. Shuttle Embodiment with Ambient Pressure and Pump as Basic Design

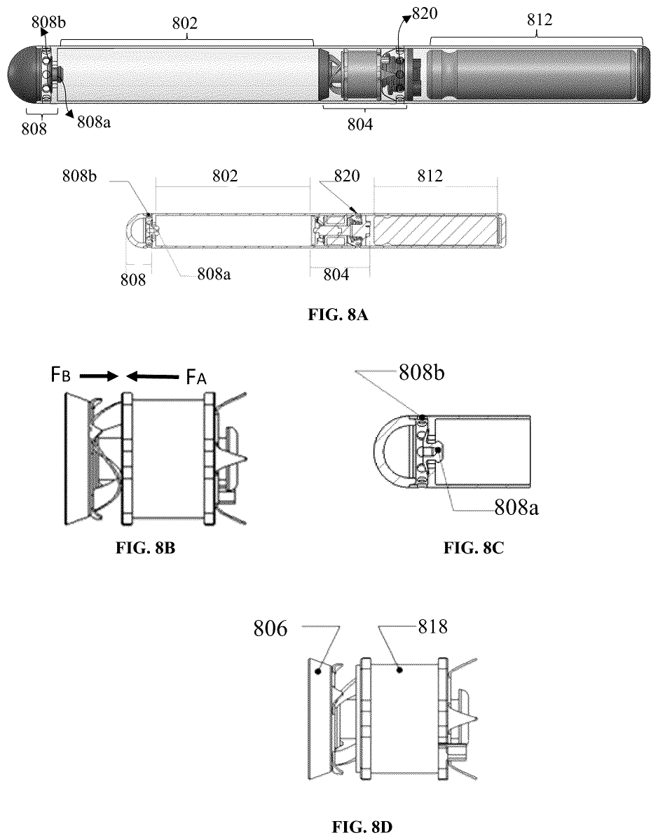

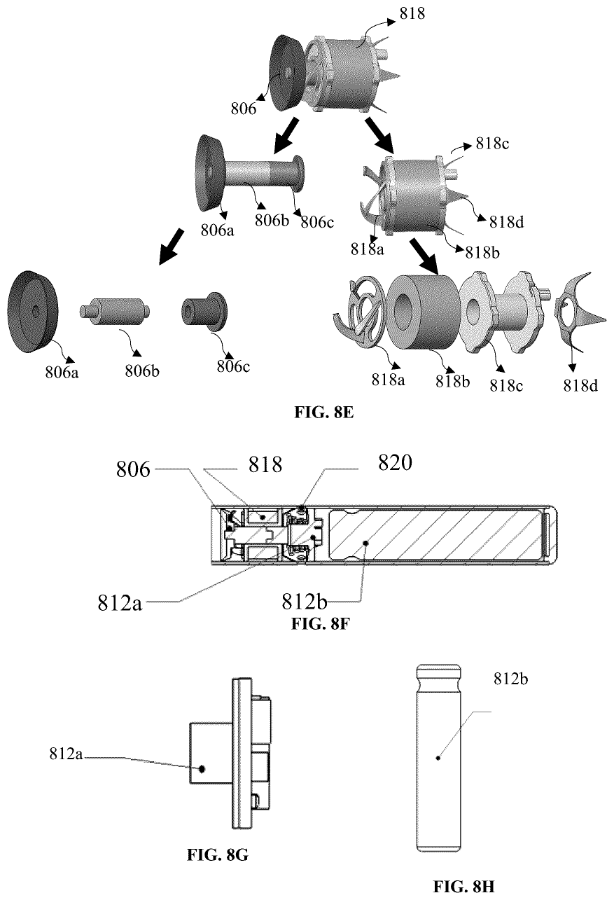

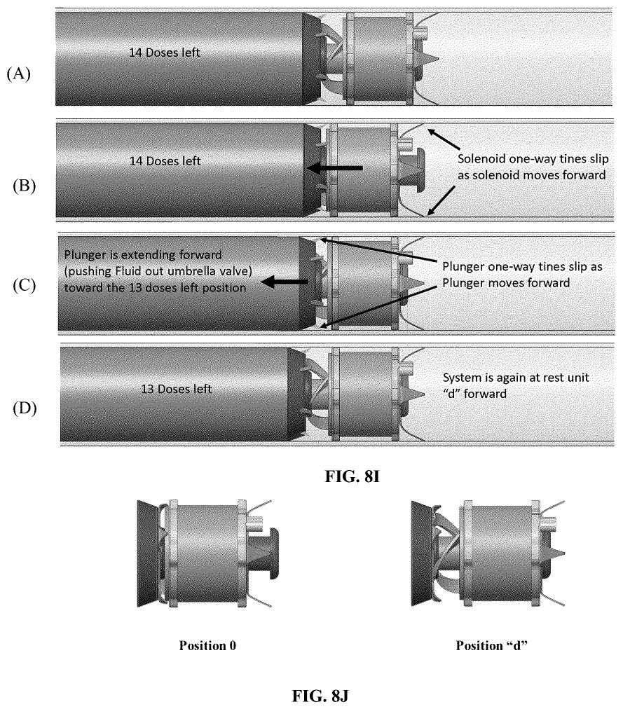

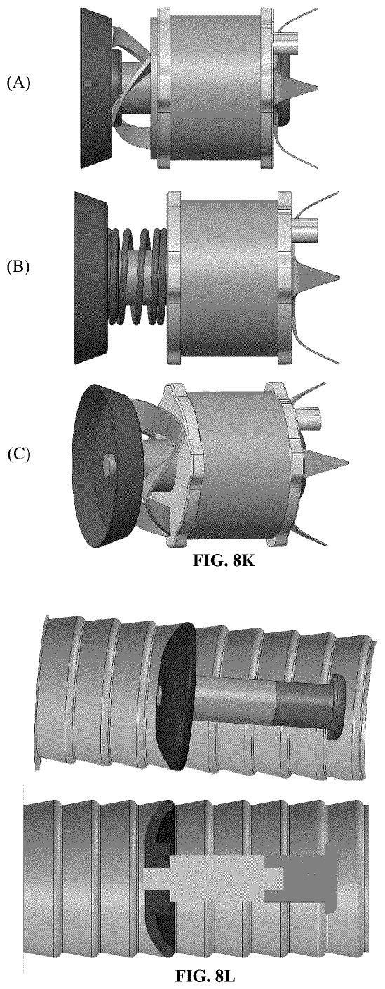

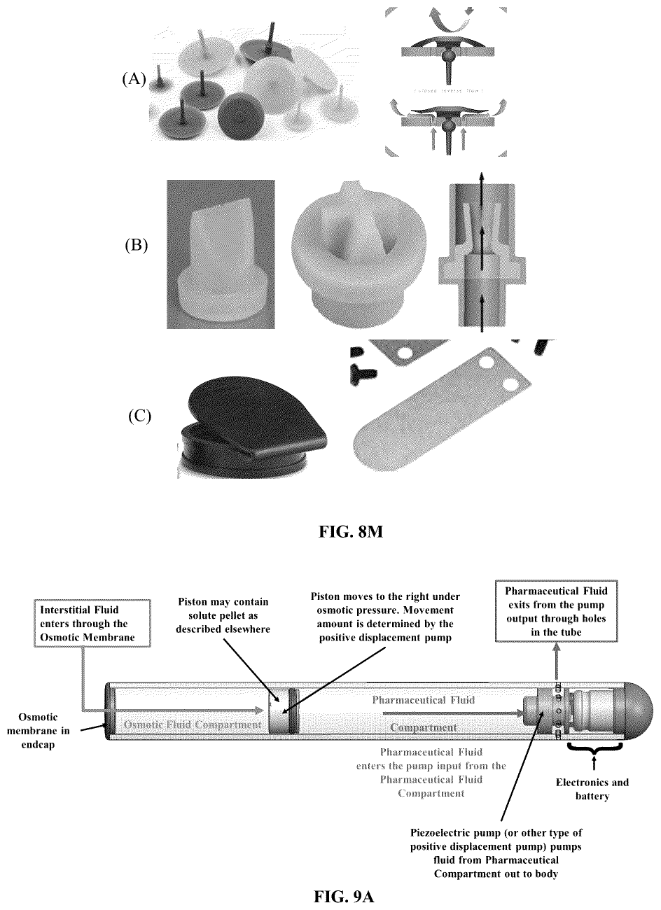

FIG. 8A illustrates a transparent side view of the implantable device where subassemblies of the implantable device are visible, according to one or more embodiments. The implantable device comprises a drug reservoir unit 802, a displacement unit 804, a drug delivery unit 808, and an electronic unit 812. The drug reservoir unit is fluidically connected with the drug delivery unit via a one-way valve. The displacement unit provides a force on the drug fluid that is enough to open a valve 808a present in the drug delivery unit 808 to release a fixed amount of drug via a plurality of fluid exit holes 808b. A plurality of fluid entrance holes 820 distributed radially allows inflow of body fluid into the space created by the displacement unit as it pushes the drug toward the one-way valve. The electronics may be potted, glued, or otherwise mounted within the implantable device, and are separated and sealed from the rest of the subassemblies via a seal. In an example, the electronic unit 112 may be potted to the first component of the implantable device comprising the drug reservoir unit 102, the displacement unit 104, and the drug delivery unit 108, but “behind” the plurality of fluid entrance holes 120 so as not to obstruct bodily fluid entrance as the displacement unit 104 moves forward toward the valve. Potting is a method used to protect circuit boards (referred to in these contexts as the substrate) by filling a PCB enclosure with a liquid material called a potting compound or encapsulation resin. The potting compound fills the device's housing and, in most cases, covers the entire circuit board and its components, although in some cases it can be used to pot individual components. Potting provides excellent abrasion resistance, as well as protection against heat, chemicals, impacts, and other environmental hazards. Typical potting compound materials include epoxy, polyurethane, and silicone compounds. The battery may be potted as well and may be potted together with the PCB. The PCB and batteries may also be isolated via walls or other methods of keeping them separate.

In an embodiment, variability per shot of a drug by the implantable device preferably could be ±15% by volume. In some embodiments, per shot variability of a drug by the implantable device, more preferably could be ±10% by volume. In some embodiments, variability per shot of a drug by the implantable device most preferably could be ±5% by volume.

Referring to FIG. 8B, the figure illustrates as an example forces acting in the implantable device to deliver a consistent volume of drug per shot, according to one or more embodiments. In an example, the forces can be visualized as F_{A} and F_{B} that act in the implanted device to deliver a consistent volume of drug to the animal or the subject's body, at each shot like that of an injection. F_{A} is the solenoid force and F_{B} is the spring force. The device is designed to overcome the frictional forces (including initial friction) and viscosity effects of the fluid (drug) so that the solenoid coil assembly counteracts the spring force. The forces for the entire device are described elsewhere in this document. The solenoid is driven by a Printed Circuit Board (hereafter PCB) through electrical wires. A coil of wire from the PCB to the solenoid may be in a compressed state. The electrical wire coil attached to the solenoid and the PCB stretches out as the shuttle moves away from the PCB toward the drug delivery unit. The PCB remains stationary and is sealed in place. An inert bumper can be used to keep the coil of electrical wire from tangling before the coil starts moving. The inert bumper can alternately be a functional component on the PCB. When power is supplied to the coil of the shuttle solenoid, the shuttle moves towards the drug delivery unit, uncoiling the wire between the shuttle and the PCB as it moves. A capacitor may be charged up by the circuitry and released to the coil in a short, but powerful, burst. This burst can be used to help overcome the initial friction of components including the piston as well as assist in overcoming sticking forces of the one-way valve. This may also allow the system to operate using significantly lower power components and may allow a smaller battery. Alternately, the electronics and battery may be attached to the solenoid and move with the solenoid. In this embodiment, the inlet holes allowing bodily fluids to enter would not only be “behind” the solenoid but may be behind the electronics and battery as well. The inlet holes may be in the endcap as well.

In some embodiments, the inlet holes allowing interstitial fluid to enter the device may be filtered. Additionally, the filters may be made from or coated with a material known to prevent or reduce clotting or buildup of proteins.

FIG. 8C illustrates a close view of the drug delivery unit of the implantable device, according to one or more embodiments. In an example, the drug delivery unit 308 comprises a valve 308a and a drug delivery orifice. When a threshold pressure is exerted by the piston on the valve 308a, the valve 308a releases the pharmaceutical drug which comes out the drug delivery orifice cut into the tube ahead of the valve. In an example, the drug delivery orifice comprises a plurality of fluid exits 308b. In an example, the plurality of fluid exits are radially distributed around the drug delivery unit to reduce clogging during insertion. The drug can disperse radially from the drug delivery unit to the body of the subject having the device implanted. The number of and the shape of the exit holes will depend on the structure of the device, the material of the device, quantity, viscosity, and consistency of a drug to be delivered to the subject, as well as the rate of drug delivery.

FIG. 8D and FIG. 8E illustrate exploded views of the displacement unit of the implantable device, according to one or more embodiments. The displacement unit comprises a solenoid coil. The core of the solenoid is attached to a piston. When the solenoid is activated, the piston and solenoid are pulled together against the spring force. In an example, in FIG. 4B, the shuttle (or the electromagnetic actuator) comprises a piston unit 406 and a solenoid coil 418. The piston unit 406 further comprises a piston 406a, a solenoid piston core 406b, and a piston stop 406c. The solenoid piston core 406b is a ferromagnetic core that is pulled to the magnetic coil center of the solenoid coil 418 when coil 418 is powered. The piston stop 406c prevents the solenoid piston core 406b from travelling too far forward. One-way tines may be added to the piston 406c to prevent the piston 406c from moving backwards as the solenoid is pulled forward. The piston is generally prevented from moving backwards by the closed valve 408a as it prevents fluid from moving backwards in the tube. Tines on the solenoid keep the solenoid from moving backwards in the tube as well. The tines on the coil and/or the piston provide an effective way of preventing the shuttle from moving backwards at any time. The tines may be metal, a strong plastic or other material. Note that the tines on the piston may be either just behind the piston or on the piston stop. Any known means of preventing rearward motion of the shuttle or piston can be used. The solenoid coil comprises a spring 418a, a conductive coil 418b, a bobbin 418c to hold the conductive coil, and one-way tines 418d. The one-way tines 418d may be spring steel flexed outward to touch the inside the tube. The tine length, width and spring constant of the tine material determine the effective movement force and holding force. The displacement unit 404 (shown in FIG. 1) is configured to only move towards the drug delivery unit 102 (shown in FIG. 1). Neither the solenoid, nor the piston can move backwards. Optionally, a latching solenoid may be used to eliminate the need for a spring. This adds complexity to the electronics, and generally increases the energy demand, but may be offset by the removal of the spring.

FIG. 8F, FIG. 8G and FIG. 8H illustrate a close view of the example components of the electronic unit of the implantable device, according to one or more embodiments. The electronic unit 512 (shown in FIG. 8G may comprise a power component 512a (FIG. 5B) and a control component 512b (FIG. 5C). The PCB is connected to battery (FIG. 512b) for power. In an example, the electronics/battery compartment may be potted into the implantable device. Other power sources besides that of a battery may be used as well as described elsewhere in this document.

The primary forces through solenoid retraction and extension are: Solenoid force (Fsld), Spring force (Fspg), Piston static friction (Fplg-s), Piston kinetic friction (Fplg-k), Valve static opening force (Fval-s), Valve opening force (Fval-o), Shuttle Tines static force (Fstin-s), Shuttle Tines kinetic friction (Fstin-k), Piston Tines static force (Fptin-s), Piston Tines kinetic friction (Fptin-k), Fluid Viscosity force (Ffld), and Shuttle momentum (Fsm). Fsld is active in solenoid extension only and varies as the piston extends from 0 to d. Fspg varies as the piston extends from start position (0) to end position at distance d depending on spring type. Fplg-s is due to friction against the tube wall during initial movement. Fplg-k is due to friction against the tube wall while moving. Fval-s is an initial valve “sticking” force. Fval-o is the force on the one-way valve required for fluid flow. Tines may initially stick against tube due to Fstin-s. Fstin-k is due to friction against the tube wall while moving. Tines may initially stick against the tube due to Fptin-s. Fptin-k is due to friction against the tube wall while tines are moving. Ffld is an amount of force required to overcome pharmaceutical viscosity. Fsm is an amount of force required to overcome initial momentum.

FIG. 8I illustrates a process of the pumping action of the implantable device, according to one or more embodiments. (a) of FIG. 8I depicts the shuttle at rest. When the system is at rest, the spring holds the piston, solenoid piston core, and piston stop in the forward position. These components cannot move further forward due to the piston stop. (b) of FIG. 8I depicts the shuttle under electronic force. When the solenoid is powered, the solenoid moves forward compressing against the spring. The solenoid moves forward because the piston cannot move backward due to both the fluid held in place in the drug reservoir unit and due to the closed one-way valve. Optionally, flexible tines on the piston may also prevent backward motion of the piston. Effectively, since the piston is pulled toward the coil and the piston cannot move, the coil moves forward. (c) of FIG. 8I depicts the shuttle when electronic force is removed. When power is removed from the solenoid, the spring pushes the piston forward. The solenoid cannot move backwards due to the solenoid tines. The tines are spring loaded at an angle against the sides of the tube preventing rearward motion. (d) of FIG. 8I depicts the shuttle in release state. Once the spring has moved the piston forward and the fluid is expelled through the valve, the piston stop prevents further movement forward of the piston and the system is again at rest. The cycle repeats as needed to provide the appropriate dose.

Electrical wires protruding from the solenoid coil to the PCB provide power to the solenoid coil. These wires uncoil as the displacement unit moves forward within the tube. The distance between the piston and the solenoid is ‘d’. When the solenoid power releases (is turned off), the spring slowly pushes the piston into the drug reservoir unit, forcing the pharmaceutical out of the implantable device by way of the drug delivery unit. Every time the solenoid coil is powered and unpowered, the piston moves forward by a distance ‘d’ within the tube. The control unit of the electronic unit defines the periodicity of the solenoid coil firing and thus the drug is released via the implantable device at the desired intervals. The tines keep the Shuttle (and piston if tines added to the piston) from moving backwards in the tube. Any other means of preventing rearward motion of the Shuttle or Piston can be used as well.

FIG. 8J illustrates forces acting in the implantable device, according to one or more embodiments. There are two actions that cause forces on the implantable devices of FIG. 8A: solenoid retraction and solenoid release. These actions are shown in FIG. 8I. The forces vary throughout the travel of the piston as it moves from Position 0 (fully retracted) to Position “d” (fully extended) as illustrated in FIG. 8J. Position d can change in the design if a dose change is required. Additionally, the dose may be reduced on a given device by reducing the power to the solenoid so that the piston pushes less than distance “d”. The solenoid must overcome all other forces during solenoid retraction i.e., Fsld should be greater than Fspg+Fplg-s+Fplg-k+Fval-s+Fval-o+Fstin-s+Fstin-k+Fptin-s+Fptin-k+Ffld+Fsm. Since Fplg-s, Fplg-k, Fval-s, Fval-o, Fptin-s, Fptin-k, and Ffld are zero or near zero during solenoid retraction, this roughly equates to Fsld should be greater than Fspg+Fstin-s+Fstin-k+Fsm. The spring must overcome all other forces during extension i.e., Fspg should be greater than Fsld+Fplg-s+Fplg-k+Fval-s+Fval-o+Fstin-s+Fstin-k+Fptin-s+Fptin-k+Ffld+Fsm. Since Fsld, Fstin-s, Fstin-k, and Fsm are zero or near zero during spring extension, this roughly equates to Fspg>Fplg-s+Fplg-k+Fval-s+Fval-o+Fptin-s+Fptin-k+Ffld. As most of the initial (startup) forces happen at the start of solenoid retraction, a capacitor may be incorporated to provide an initial energy pulse to the solenoid coil when countering forces (startup forces) are the greatest. The capacitor would be charged up from the battery before energy is provided to the solenoid coil. Optionally, the charging voltage to the capacitor could be increased to a significantly higher voltage through the use of a voltage doubler, tripler, or other voltage boosting circuit.

Referring to FIG. 8K, the figure illustrates sample spring embodiments for the displacement unit of the implantable device. The non-limiting examples of springs can be any of A) tab spring B) compression spring, and C) a wave disc spring. The piston spring pushes against the solenoid force and can be one of many types of spring. Optionally, the spring may be located on the piston stop side of the solenoid.

In an embodiment, the implantable device comprises a flexible umbrella shaped piston. The flexible (possibly silicone) piston locks in place at each step “d”. The scalloped design of the walls of the tube prevents the piston from moving backwards. No piston tines are needed to stop rearward motion.

FIG. 8L illustrates an umbrella piston of the implantable device, according to one or more embodiments. The umbrella piston pumping action is exactly like the illustration provided in FIG. 8I, except that the umbrella plunger moves forward a discrete amount by locking into the scalloped ridges of the tube. When the solenoid activates, the displacement unit moves forward and when the solenoid is inactivated, the spring force on the plunger moves the plunger forward towards the valve by unit “d” into the next ridge in the tube.

Referring to FIG. 8M, the figure illustrates a sample valve of the implantable device. The non-limiting examples of the valve are A) umbrella valve, B) duckbill valve, and C) flapper valve. In an embodiment, the device may comprise a plurality of seals (for example a seal at the piston and a seal at the valve). The electronics are separated and sealed from the mechanical systems of the implantable device possibly using a seal.

Implantable Drug Delivery System Having a Displacement Pump with or without an Osmotic Pump

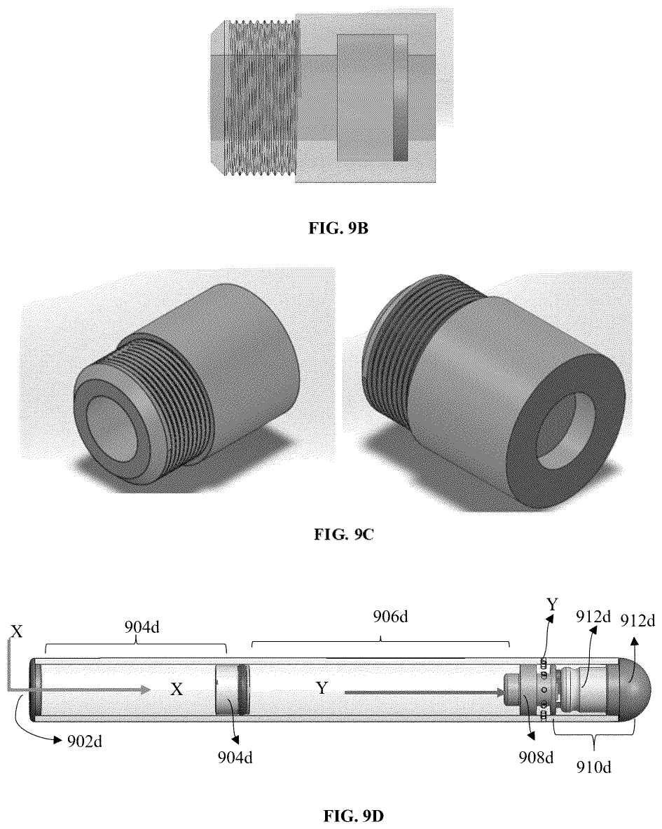

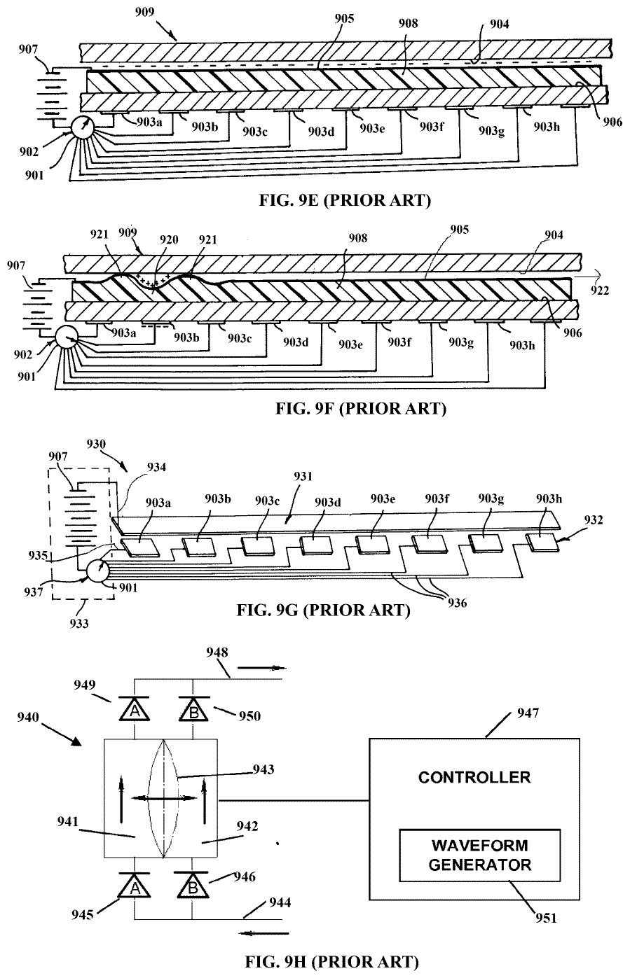

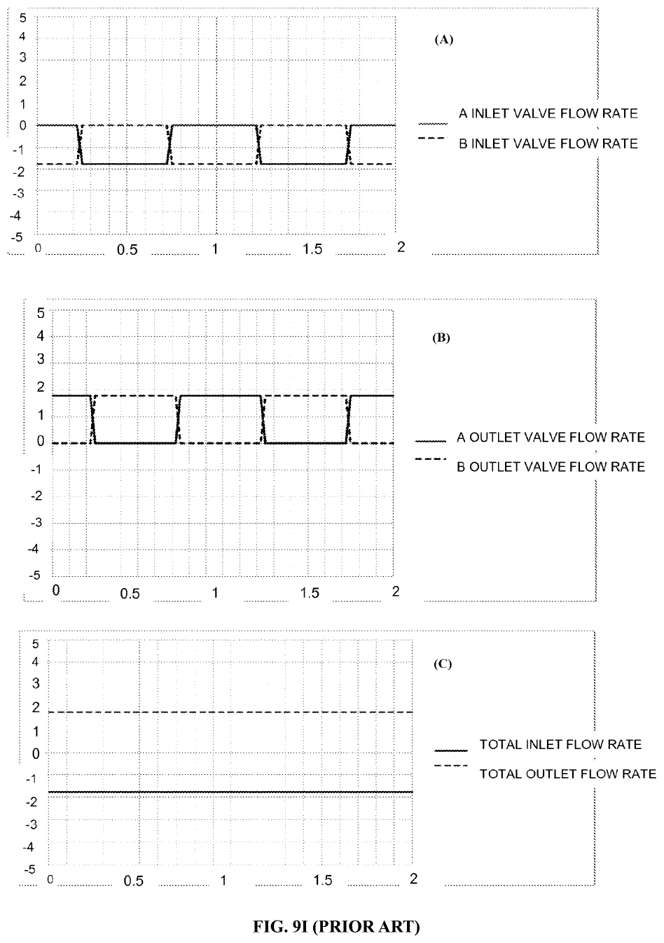

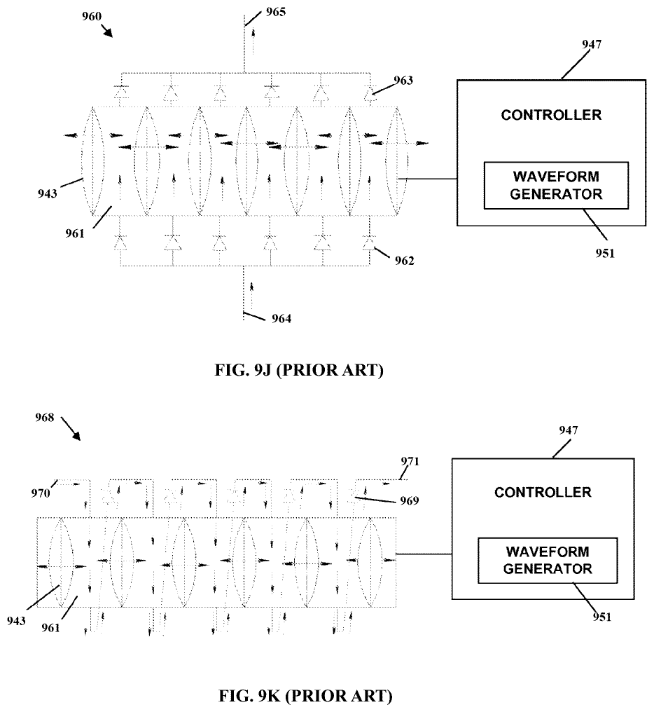

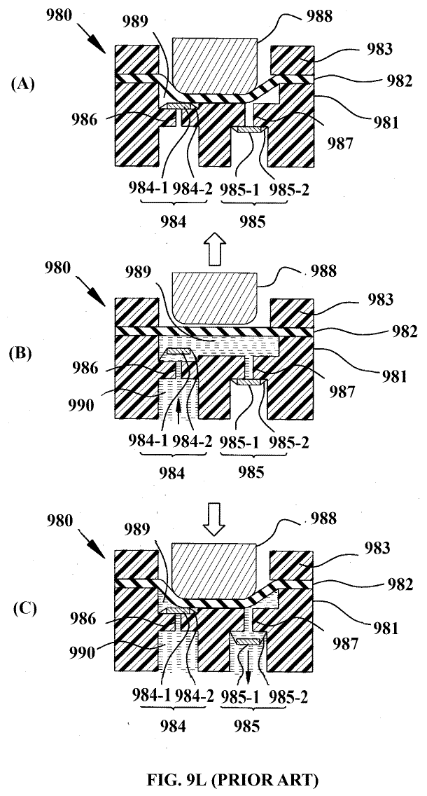

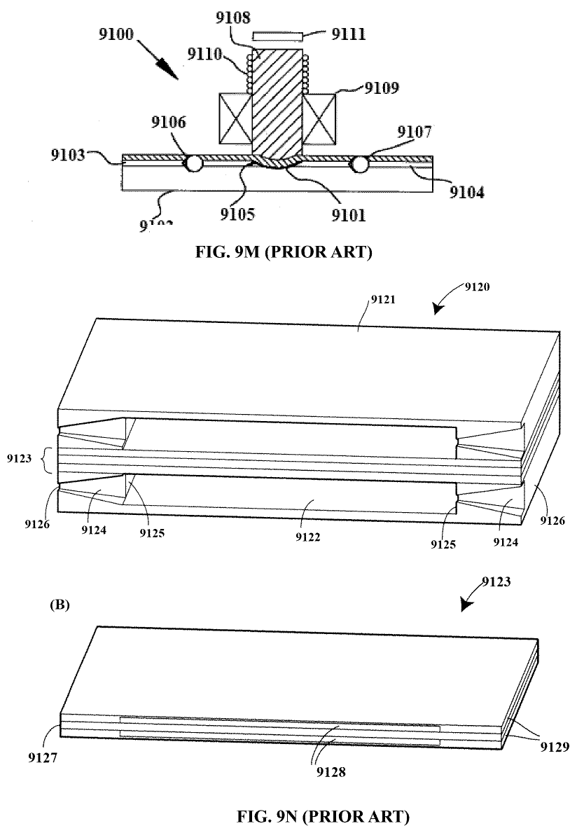

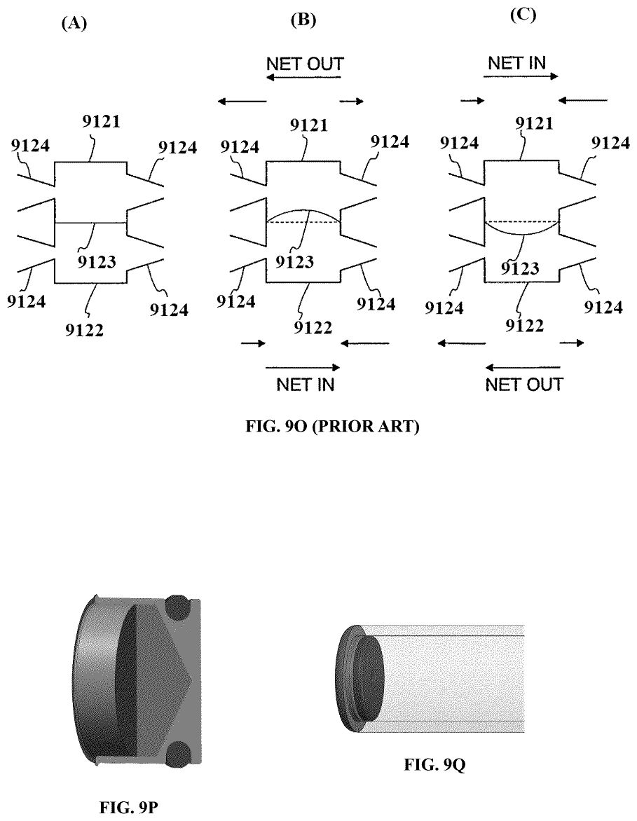

Referring to FIG. 9A, it shows a schematic of an osmotic pump based implantable device comprising a positive displacement pump, according to one or more embodiments. The interstitial fluid enters through the osmotic membrane to the osmotic fluid compartment. The piston may contain osmotic substance that dissolves with the interstitial fluid forming an osmotic solution. This osmotic solution will create an osmotic pressure on the piston due to which the piston will move towards the drug reservoir unit (Pharmaceutical fluid compartment). A positive displacement pump (for example a piezoelectric pump) can be used to dispense the pharmaceutical fluid out of the device into the body of the subject. The pumping of the positive displacement pump and therefore dispensing of a drug can be controlled using electronics of the device. The piston movement amount will be determined by the positive displacement pump.

In another embodiment, the osmotic membrane within the endcap can slide within a chamber. The osmotic membrane may be held within a sliding frame to support the membrane. The chamber volume may be equal to the desired volume of the pharmaceutical dose. In one embodiment, this endcap is threaded so that it can be screwed on and off of the pharmaceutical chamber. During times when the pump is not active, the osmotic pressure will push the slidable osmotic membrane all the way toward the endcap. The sliding motion of the osmotic chamber allows the pump to pull the membrane forward thus enable the pump to pull the entire pharmaceutical dose without having to wait for interstitial fluid flow through the membrane which is typically slow compared to pumping speeds.

Referring to FIG. 9B and FIG. 9C, FIG. 9B shows a transparent sideview of the Sliding Osmotic Membrane Endcap and FIG. 9C orthogonal views of Sliding Osmotic Membrane Endcap.

### Non-Osmotic Pump-Based Drug Delivery Device

In an embodiment, a non-osmotic pump based implantable drug delivery device is provided. In this embodiment, the drug is held at zero pressure in a drug reservoir chamber of the device as compared to the surrounding. The pharmaceutical fluid is pumped by a positive displacement pump from the drug reservoir chamber through the pump, out to the body. A piston is pulled to the right as the pump pulls fluid from the drug reservoir chamber. As the piston gets pulled towards the positive displacement pump, the interstitial fluid enters into the device via one or more small holes to fill the space. This embodiment does not employ osmotic pressure. Any positive displacement pump may be used for the pump including piezoelectric, piston or diaphragm pumps. A piston position sensor may be added as an addition check on dosing amount. In some embodiments, the device may comprise a valve in combination with the pump. The valve that can be used within this device are described elsewhere in this application. In some embodiments, valves can be used within this device as a passive valve, for example internal to the pump.

Referring to FIG. 9D, the device comprises a fluid chamber 804, a drug reservoir chamber 806, an electronics chamber 910, a positive displacement pump 808, and a sliding piston 804 slidable towards the positive displacement pump 808. The interstitial fluid X enters through one or more inlet holes via a first endcap 802. The first endcap 802 may or may not comprise a membrane filter. The drug reservoir chamber 806 comprises a pharmaceutical fluid composition Y. The pharmaceutical fluid Y can enter the input of positive displacement pump 808 from the drug reservoir chamber 806. The positive displacement pump 808 pumps the pharmaceutical fluid Y from the drug reservoir chamber 806 out of the body. The electronics chamber 810 comprises one or more electronics (explained somewhere else in this application) and a battery 812a that controls and powers the pump. The piston 804 moves towards the positive displacement pump 808 as the pump pulls the pharmaceutical fluid Y out of the device. The second end cap 812 secures the electronics into the device.

In an embodiment, variability per shot of a drug by the implantable device preferably could be ±15% by volume. In some embodiments, per shot variability of a drug by the implantable device, more preferably could be ±10% by volume. In some embodiments, variability per shot of a drug by the implantable device most preferably could be ±5% by volume.

Below are described example pumps that can be used in the implantable drug delivery device with or without an osmotic pump:

### 1. Electrostrictive Micro-Pump (Peristaltic Micro-Pump)

In an embodiment, it is an electrostrictive micro-pump is provided for controlling a fluid flow through a cannula or other narrow liquid conduit. The micro-pump includes a pump body having a passageway for conducting a flow of fluid, a pump element formed from a piece of viscoelastic material and disposed in the passageway, and a control assembly coupled to the viscoelastic material for electrostatically inducing a peristaltic wave along the longitudinal axis of the pump element to displace fluid disposed within the pump body. The control assembly includes a pair of electrodes disposed over upper and lower sides of the pump element. The lower electrode is formed from a plurality of uniformly spaced conductive panels, while the upper electrode is a single sheet of conductive material. A switching circuit is provided for actuating the conductive panels of the lower electrode in serial, multiplex fashion to induce a peristaltic pumping action.

In FIG. 9E, the multiplexer 901 of the switching circuit 902 applies no electrical potential to any of the conductive panels 903a-h. Hence there is no pressure applied to any liquid or other fluid present in the space between upper inner wall 904 of the cannula and the flexible layer of conductive material 905 that forms the upper electrode 906. When the micro-pump is actuated, the multiplexer 901 first connects conductive panel 903a to the bottom pole of the DC power source 907. This action generates an electrostatic force between the panel 903a and the portion of the flexible, conductive material 905 immediately opposite it. The pump body 909, which, in this example, is connected to a source of liquid i.e., the drug chamber. In an embodiment, it may be placed inside the drug chamber.

As shown in FIG. 9F, a resulting electrostatic attraction creates a pinched portion 920 in the viscoelastic material forming the pump element 908. As a result of the law of conservation of matter, an enlarged portion 921 is created immediately adjacent to the pinched portion 920. Though for illustration activation of 903b is shown, the multiplexer 902 proceeds from activation 903a. The multiplexer 902 proceeds to disconnect the panel 903b from the DC power source 907 and to subsequently connect the next adjacent conductive panel 903c to the source 907. This action in turn displaces both the pinched portion 920 and enlarged portion 921 of the viscoelastic pump element 908 incrementally to the right. The sequential actuation of the remaining conductive panels 903c-h effectively propagates the enlarged portion 921 toward the right end of the pump element 908. As the peak of the enlarged portion 921 contacts the upper inner wall 904 throughout its rightward propagation, the pump element 908 peristaltically displaces the small volume of liquid disposed between the layer 905 and the upper wall 904 of the pump 909, thereby generating a pressure that causes liquid to be expelled out of the outlet 922.

It should be noted that the displacement of the micro-pump may be adjusted by preselecting the volume in the cannula between the upper layer 905 forming the upper electrode 906 and the upper inner wall 904 of the cannula passageway. The rate of fluid displacement may be controlled by adjusting the frequency of the multiplexer 901. To compensate for the inherently lower amplitude of the enlarged portion 921 in the pump element 908 at higher frequencies, the voltage generated by the DC power source may be increased so that the peak of the resulting enlarged power 921 engages the upper inner wall 904 during its propagation throughout the length of the pump element 908.

One of the advantages of the micro-pump of the invention is that the pumping action may be positively stopped by applying an electrical potential simultaneously to each of the conductive panels 903a-h. In a particular operation of the invention the multiplexer 901 applies a voltage from the DC power source 907 to all the panels 903a-h, multiple static pinched portions 920 are created which in turn create multiple static enlarged portions 921 which engage the upper wall 904 of the cannula passageway. As a result of such operation, the pump element 908 effectively becomes a viscoelastic valve element which positively prevents the flow of further liquid from the vented liquid source through the outlet 922. The capacity of the micro-pump to simultaneously function as a flow restricting valve advantageously obviates the need for the construction and installation of a separate microvalve to control the flow.

While this invention has been described in terms of several preferred embodiments, various modifications, additions, and other changes will become evident to persons of ordinary skill in the art. For example, the micro-pump could also be constructed by mounting two pump elements 908 in opposition on the upper and lower walls of the cannula passageway. Each valve element 908 could have its own separate control assembly, and the operation of the two control assemblies could be coordinated such that complementary peristaltic waves were generated in the two different pump elements. In another embodiment, the pump element 908 may have pre-formed shape to act as a usually closed path, but open on acting of the multiplexer 901. Such a modification would have the advantage of a greater liquid displacement capacity. All such variations, modifications, and additions are intended to be encompassed within the scope of this patent application, which is limited only by the claims appended hereto and their various equivalents.

With reference again to FIG. 9G, the control assembly 930 includes upper and lower electrodes 931 and 932 which cover upper and lower surfaces of the valve element in sandwich-like fashion. Electrodes 931 and 932 are in turn connected to a voltage source 933 of electrical voltage via conductors 934, 935 which may be metallic strips fabricated via CMOS technology. The upper electrode 931 may be formed from a thin layer of a flexible, conductive material applied to the upper surface of the pump element 908 by vapor deposition or other type of CMOS-compatible coating technology. Examples of conductive materials which may be used for the conductive layer on the pump element 908 includes electrically conductive polymers such as polypyrrole, polyanaline, and polythiophene. Alternatively, a relatively non-reactive metal such as gold, silver, or nickel may be used to form the layer layer on the pump element 908. Of course, other conductive metals such as aluminum could also be used but less reactive metal coatings are generally more preferred, since they would be able to interface with a broader range of liquids without degradation due to corrosion. Finally, electrically conductive, diamond-like carbon might also be used. In all cases, the thickness of the layer may be between 0.2 and 1 micron thick. The lower electrode 932 may be formed from the same material as the upper electrode 931. However, as there is no necessity that the lower electrode 932 be flexible, it may be made from thicker or more rigid electrically conductive materials if desired. Lower electrode 932 includes a plurality of conductive panels 903a-h electrically connected in parallel to the electrical voltage source 933 via conductive strips 936 which again may be formed via CMOS technology.

The electrical voltage source 933 includes a DC power source 907. One of the poles of the DC power source is connected to the upper electrode 931 via conductor 934, while the other pole of the source 907 is connected to the lower electrode 932 via conductor 935 and switching circuit 937. Switching circuit 937 includes a multiplexer 901 capable of serially connecting the conductive panels 903a-h of the lower electrode 932 to the DC power source 933 at frequencies up to 12.5 kHz.

### 2. Micro Pump for Microfluidic Channel

Micro Pump for microfluidic channel with actuating element is based on one or more of piezoelectric, thermal, electrostatic or electromagnetic transduction.

A micro pump is formed on a substrate having a common inlet channel and a common outlet channel by a plurality of pumping elements, each pumping element having an inlet coupled to the common inlet channel and an outlet coupled to the common outlet channel, the inlet and outlet connected by a microfluidic channel, the microfluidic channel comprising a valvular conduit having low fluid flow resistance in a direction from the inlet to the outlet and high fluid flow resistance in a direction from the outlet to the inlet, and an actuating element arranged to cause fluid to be pumped through the microfluidic channel from the inlet to the outlet, wherein the actuating element is based on one or more of piezoelectric, thermal, electrostatic or electromagnetic transduction. A controller is coupled to actuate the actuating elements at mutually staggered relative timing so as to produce a substantially continuous steady flow.

FIG. 9H shows a dual-channel pump according to one embodiment of the present invention. FIG. 9H shows a dual-channel pump 940, having a pair of parallel channels 941 and 942 having a common wall 943. An inlet 944 is coupled to each of the channels 941 and 942, via valves 13 and 14, respectively and an outlet 18 is coupled to each of the channels 941 and 942 via valves 945 and 946. In this case, as will be appreciated, when the common wall 943 is flexed in one direction under the control of a controller 947, for example to the left, then the right-hand channel 942 increases in volume and valve 946 allows fluid to pass into the right-hand channel 942 from the inlet 944, while valve 950 isolates the right-hand channel 942 from the outlet 948. At the same time, left-hand channel 941 is reduced in volume, causing fluid to pass therefrom through the valve 949 to the outlet 948, while valve 945 prevents fluid flow therethrough back to the inlet 944. When the common wall 943 is flexed in the opposite direction, i.e. to the right, the opposite happens, so that the left-hand channel 941 increases in volume and valve 945 allows fluid to pass into the left-hand channel 941 from the inlet 944, while valve 949 isolates the left-hand channel 941 from the outlet 948. At the same time, right-hand channel 942 is reduced in volume, causing fluid to pass therefrom through the valve 950 to the outlet 948, while valve 946 prevents fluid flow therethrough back to the inlet 944.

If the common wall 942 is actuated by the controller 947 to flex in a normal, sinusoidal fashion from one side to the other, the inlet flow rates through the two inlet valve 945, 946 will be in opposite phase to each other, as the common wall 942 flexes from one side to the other. The non-return valves may be replaced by fluidic diodes. Fluidic diodes are non-return valves that have no moving parts and are manufactured in silicon using micro machining processes, to form Micro Electrical Mechanical Systems (MEMS). A fluidic diode is a device that allows fluid to flow in one direction but not in the opposite direction, similar to an electronic diode that allows current to flow in one direction but not in the opposite direction. The fluidic diode works by using channels or chambers that are designed to create resistance to fluid flow in one direction, while allowing free flow in the other direction. Fluidic diodes can be created using a variety of methods and materials, such as microfabrication techniques or soft lithography, and can be used in a variety of applications, such as microfluidic systems, drug delivery devices, and fluid control systems. Some fluidic diodes rely on passive structures, while others may use active elements, such as pumps or valves, to control fluid flow. Fluidic diodes are useful where the directional flow of fluids needs to be controlled or where fluid flow needs to be regulated.

Thus, the fluidic diodes 945, 946, 949 and 950 are symbolically represented by an electrical diode symbol, in order to distinguish them from the mechanical non-return valves. They often comprise a plurality of topological micromixers that split, turn, and recombine the fluid arranged in series in the fluidic diode. There are a number of such fluidic diodes available.

Furthermore, the controller 947 includes a waveform generator 951 to enable the controller to control the common wall to be moved according to a different input waveform other than the standard sinusoidal signal. In one embodiment, the waveform generator 951 generates a triangular-shaped waveform. In this case, the inlet flow rates through the two inlet fluidic diodes (A & B) 945 and 946 will again be in opposite phase to each other, as the common wall 942 flexes from one side to the other, as shown in FIG. 9I (A), with FIG. 9I (B) showing the outlet flow rate through the two outlet fluidic diodes (A & B) 949, 950 also in opposite phase to each other, and to their respective inlet fluidic diodes. FIG. 9I (C) shows the total inlet and outlet flow rates as the combination of the flow rates through the inlet fluidic diodes and the outlet fluidic diodes, respectively, showing that, with a triangular-shaped actuation waveform, the input and output flow rates are no longer part-sinusoidal but are substantially constant. The overall static pressure in the external circuit, being a combination of the total inlet and outlet flow rates, is zero.

As will be described further below, triangular-shaped actuation waveforms are not the only waveforms that will produce substantially constant input and output flow rates. For example, trapezoidal and parabolic waveforms will also produce substantially constant input and output flow rates.

FIG. 9I shows a multi-channel pump 960 with a multiplicity of parallel pumping channels 961. In the drawing, six parallel pumping channels are shown, but it will be appreciated that more channels could be utilized as part of a larger array. As shown, each pumping channel 961 is connected to an inlet 964 via a respective inlet fluidic diode 962, and to an outlet 965 via a respective outlet fluidic diode 963. Again, waveform generator 951 generates a control waveform for controller 947 to control walls 943 between adjacent channels 961 in a two-phase mode, such that every second wall 943 is flexed on one direction and alternate walls 943 are flexed in the other direction, so that alternate channels are either compressed or expanded to force fluid out or in, respectively.

FIG. 9K shows a multi-channel pump 968 with a multiplicity of series pumping channels 961. In the drawing, six parallel pumping channels are shown, but it will be appreciated that more channels could be utilized as part of a larger array. As shown, each pumping channel 961 is connected, via a respective fluidic diode 969, to an outlet of the preceding pumping channel 961.

The first pumping channel is connected to an inlet 970 and the final pumping channel 961 is connected to the outlet 971. Again, a waveform generator 951 generates a control waveform for controller 947 to control walls 943 between adjacent channels 961 in a two-phase mode, such that every second wall 943 is flexed on one direction and alternate walls 943 are flexed in the other direction, so that alternate channels are either compressed or expanded to force fluid out or in, respectively.

The electronic drive circuits forming the controller and the waveform generator can be realised using well-known techniques. However, the circuits will be required to take the particular voltage versus time profile definitions and to convert these faithfully to the levels of voltage and current required to cause the volume displacement elements to move as needed.

As used herein, the term “waveform” means the profile of voltage versus time applied by drive electronics forming the controller to piezo-electric or other types of actuators. It exploits the fact that because the piezo actuators behave linearly, wall displacements are proportional to voltages applied. The waveforms will, in general, be periodic in nature and will have the same profile from channel to channel. In a two-phase mode, every other channel will be in phase, whilst the neighbour channels in between will be 180 degrees (or Pi Radians) out of phase. In a three-phase arrangement, every third channel will be in phase, whilst the neighbour channels in between will be 120 degrees and 240 degrees (or 2*Pi/3 and 4*Pi/3 Radians) out of phase. In a four-phase arrangement, every fourth channel will be in phase, whilst the neighbour channels in between will be 90 degrees, 180 degrees and 270 degrees (or Pi/2, Pi and 3*Pi/2 Radians) out of phase.

The waveform profiles are preferably designed to ensure that at any given instant, the total volume displaced from all of the phases combined is zero, or very close to zero. This ensures that the static pressure in the pumped system remains substantially constant. Beneficially, the waveform profiles are designed so that the volumes of the individual chambers change linearly with time, or are kept constant; that is, the waveform profiles are either triangular or trapezoidal. This means that the rates of change of volume are either constant or zero, in turn causing the rates of flow through the respective non-return valves to be constant or zero. This, in turn, means that it is possible for flows from separate elements to be added together at all instants in time to produce an overall constant rate of flow. Triangular waveforms may be arranged such that each actuating element moves from one end of its travel to the other in half a cycle and then back again in half a cycle. Three-phase trapezoidal waveforms are preferably arranged such that each actuating element moves from one end of its travel to the other in a third of a cycle, dwells for a sixth of a cycle, moves back again in a third of a cycle and dwells for a sixth of a cycle. Four-phase trapezoidal waveforms are arranged such that each actuating element moves from one end of its travel to the other in a quarter of a cycle, dwells for a quarter of a cycle, moves back again in a quarter of a cycle and dwells for a quarter of a cycle. Sinusoidal or other regular waveforms may also be used if the application does not demand minimal levels of flow rate or pressure fluctuation.

The non-return valves can perform two related, but different, functions in a micro-pump. Firstly, they can be used to prevent reverse flows if and when all the actuating channels are switched off, for instance in either the planned or unplanned event of power being removed from the whole micro-pump. Secondly, the presence of the non-return valves allows a method of controlling flow-rate from the micro-pump.

One suitable form of actuating element that can be used to cause the fluid to move through the channels is a piezo channel array. Such actuators can easily be integrated with the fluidic diodes described above to cause the fluid to move through the channels. However other actuating elements could alternatively be used. Diaphragms or walls that flex in response to applied voltages via electrostatic actuation can be made from materials including, but not limited to, silicon or similar materials or polymeric sheets so as to displace volumes of fluid periodically. Silicon or similar materials can be made into diaphragms or walls that flex due to Joule heating and differential expansion effects, and that therefore displace volumes of fluid periodically. Electromagnetic actuation can be used to apply forces to diaphragms or walls causing them to flex and displace volumes of fluids periodically, by forming electrically conductive tracks in or on the flexing element and arranging for these to pass through a magnetic field.

### 3. Micro Diaphragm Pump

FIG. 9L (A) illustrates a micro diaphragm according to an embodiment of the present invention. Micro diaphragm pump 980 includes valve seat plate 981, diaphragm 982, diaphragm clamp 983, inlet check valve 984, outlet check valve 985, inlet channel 986, outlet channel 987, and actuator 988. Diaphragm clamp 983 fastens diaphragm 982 to valve seat plate 981, forming pump chamber 989. Inlet check valve 984 includes inlet spring 984-2 and inlet disk 984-1, while outlet check valve 985 includes outlet disk 985-1 and outlet spring 985-2. In FIG. 9L (A), micro diaphragm pump 980 is empty, and actuator 988 is in its down position. In the down position, actuator 988 pushes against diaphragm 982, making direct contact with inlet check valve 984. While actuator 988 and diaphragm 982 are in direct contact with inlet check valve 984, they provide additional sealing force between inlet check valve 984 and inlet channel 986, actively closing check valve 984. This is useful in preventing inadvertent flow through micro diaphragm pump 980 when the pump is off. Returning to FIG. 9L (A), before it has been used, micro diaphragm pump 980 contains no infusion liquid 990, and inlet check valve 984 and outlet check valve 985 are closed. In FIG. 9L (B), a pump cycle has begun. Actuator 988 is in the up position, and diaphragm 982 has moved upward, creating a drop in pressure in pump chamber 989. The drop in pressure in pump chamber 989 creates a pressure differential across inlet channel 986, stretching inlet spring 984-2 and moving inlet disk 984-1 upward. This allows infusion liquid 990 to flow through inlet channel 986, around inlet disk 984-1, and into pump chamber 989. Meanwhile, the drop in pressure in pump chamber 989 causes additional sealing force across outlet channel 987, pushing outlet disk 985-1 against outlet channel 987, and preventing flow of infusion liquid 990 from pump chamber 989 through outlet channel 987. In FIG. 9L (C), actuator 988 returns to a down position, pushing infusion liquid 990 out of pump chamber 989. As actuator 988 moves downward, pressure in pump chamber 989 increases, causing inlet disk 984-1 to seal against inlet channel 986, and pushing outlet disk 985-1 away from outlet channel 987. As outlet disk 985-1 moves away from outlet channel 987, infusion liquid 990 moves from pump chamber 989, around outlet spring 985-2 and outlet disk 985-1, and through outlet channel 987, completing a pump cycle. Actuator 988 and diaphragm 982 displace most of infusion liquid 990 from pump chamber 989. If desired, the steps illustrated in FIG. 9L (A), (B) and (C) can be repeated to deliver additional infusion liquid 990.

In terms of accuracy and delivery volume, micro diaphragm pumps are typically designed to deliver at least ±5% accuracy at both very low flow rates (such as 0.5 microliters/hr) and very high flow rates (such as 100 microliters/min). In embodiments of the present invention, sensors are often used to control and verify delivery volume from micro diaphragm pumps.

Micro diaphragm pumps, according to the present invention, are a type of positive displacement pump. In positive displacement pumps, a pump chamber is filled then emptied by action of the pump. A distinct advantage of micro diaphragm pumps (and positive displacement pumps, in general) is that they can pump gas as well as liquid, if the compression ratio is high enough. The compression ratio is the volume displaced during the actuator down stroke divided by the volume of the pump chamber. Using a micro diaphragm pump is particularly advantageous when priming the pump, since air is expelled from the pump (and its inlet and outline lines) during priming. Micro diaphragm pumps are easy for a user to set up because they can pump air and infusion liquid.

In the present invention, micro diaphragm pumps can be made using low cost, high volume manufacturing methods, including lamination, hot embossing, injection molding, and ultrasonic welding. Many different plastics can be used to achieve desired chemical and mechanical properties. Other materials, such as metal, can be used as well. In some embodiments of the present invention, metal is integrated with plastic components to produce features such as springs and electrical contacts. Thin polymer or metal layers can be laminated with thicker layers to produce moveable diaphragms and valves. In other embodiments of the present invention, components such as check valves, fluid flow channels, and diaphragms combine to form a single structure, allowing for simple manufacturing, reduced dead volume, and improved resolution and accuracy.

FIG. 9M illustrates embodiments of the present invention. Micro diaphragm pump 9100 includes diaphragm 9101, substrate 9102, inlet channel 9103, outlet channel 9104, pump chamber 9105, inlet check valve 9106, outlet check valve 9107, actuator 9108, electromagnetic coil 9109, actuator spring 9110, and sensor 9111. Inlet channel 9103 can be connected to a reservoir, which is not shown, while outlet channel 9104 can be connected to infusion lines and a cannula, which are not shown. The reservoir can be flexible or collapsible, as in the case of a plastic bag or pouch, or can be rigid, as in the case of a syringe or tube. Actuator 9108 moves up and down, making contact with diaphragm 9101, and forcing most of the infusion liquid from pump chamber 9105. As illustrated, actuator 9108 is enclosed by actuator spring 9110 and electromagnetic coil 9109, which imparts up and down motion to actuator 9108. Actuator 9108 can be used with or replaced by other elements, such as a DC motor, a piezoelectric actuator, a thermopneumatic actuator, a shape memory alloy actuator, a bimetallic strip, an ion conductive polymer film, or other components that impart up and down motion to diaphragm 9101. In some embodiments, diaphragm 9101 extends beyond pump chamber 9105 and forms the top layer of micro diaphragm pump 9100. Diaphragm 9101 can include an electrically conductive coating that forms electrical contact or capacitive coupling between diaphragm 9101, substrate 9102, actuator 9108, and/or infusion liquid that flows through the pump chamber 9105. The actuator 9108 is in its normally down position, and there is no infusion liquid in inlet channel 9103, pump chamber 9105, or outlet channel 9104. Inlet channel 9103, pump chamber 9105, and outlet channel 9104 are initially filled with air. When the actuator 9108 is in an upward position, infusion liquid is drawn through inlet channel 9103 into pump chamber 9105, and outlet check valve 9107 is closed. Infusion liquid flows through inlet check valve 9106 because a drop in pressure is created in pump chamber 9105 as actuator 9108 moves up. As a drop in pressure is created in pump chamber 9105, a pressure differential is created across inlet check valve 9106, forcing it to open. When actuator 9108 presses down on diaphragm 9101, increasing the pressure in pump chamber 9105. As pressure increases in pump chamber 9105, inlet check valve 9106 closes, and outlet check valve 9107 opens, allowing flow of infusion liquid from pump chamber 9105 through outlet check valve 9107 and outlet channel 9104. A micro bolus of infusion liquid, equivalent to the volume displaced from pump chamber 9105, is delivered through infusion lines connected to outlet channel 9104. Although most of infusion liquid is displaced from pump chamber 9105, a small amount of infusion liquid is typically left behind. The sequence is repeated, until the desired volume of infusion liquid is delivered. The shot size, or minimum deliverable volume, is approximately equal to the volume of infusion liquid that is displaced from pump chamber 9105 during the down stroke of actuator 9108. Larger volumes are delivered by cycling micro diaphragm pump 9100 multiple times. Various basal rates can be achieved by changing the up and down frequency of actuator 9108.

Actuator spring 9110 biases actuator 9108 to the down position, while activating electromagnetic coil 9109 lifts actuator 9108 to the up position, elongating actuator spring 9110. This “normally closed” configuration prevents infusion liquid from inadvertently migrating from a reservoir through inlet channel 9103 and outlet channel 9104, as can happen in the event of sudden pressure rise in the reservoir or sudden pressure drop at outlet channel 9104. Another safety feature associated with this configuration is the fact that electromagnetic coil 9109 must be pulsed on and off for micro diaphragm pump 9100 to operate. If power is accidentally applied to electromagnetic coil 9109 in a continuous (rather than pulsed) manner, actuator 9108 will remain in an up position, and infusion liquid will not be forced from pump chamber 9105. In embodiments of the present invention, solenoids and DC motors can be used as actuators, and are appealing because they produce large forces, resulting in consistent delivery even under conditions of variable backpressure, which can occur when encountering occlusion or scar tissue at the infusion site. The size of pump chamber 9105 inherently limits the amount of infusion liquid that is delivered in a single cycle, relaxing engineering constraints on the travel distance and force produced by the actuator 9108. In some embodiments of the present invention, sensors 9111 are used to indirectly detect occlusions and siphoning errors, while in other embodiments encoders are used to determine the position of the actuator 9108.

Actuator 9108 can be part of a durable, reusable system, or can be part of a disposable system. A solenoid, DC motor, or piezoelectric based actuator 9108 can be included in a durable system, along with electronics and a flexible membrane that protects durable components from ingress of water and debris, while allowing actuator 9108 to interact with diaphragm 9101. In embodiments of the present invention where a protective membrane is used, electrical contact between the durable and disposable components is optional. In embodiments of the present invention where actuator 9108 is housed with the disposable components, other actuators can be used, such as those based on thermos-pneumatic, shape memory, and piezoelectric components.

In some embodiments of the present invention, sensor 9111 can include a force sensor, contact sensor, or position sensor that works in conjunction with actuator 9108. Sensor 9111 can detect motion of actuator 9108 and confirms that micro diaphragm pump 9100 is operating as expected. If actuator 9108 is not moving when it should, sensor 9111 will detect the problem and an alarm will be activated, alerting the user to the error condition. Encoders and force sensors can be used in conjunction with actuator 9108 to verify motion, to detect bubbles in pump chamber 9105, and to detect occlusions in outlet channel 9104 (or in infusion lines and cannulas). Bubbles in pump chamber 9105 can reduce force at sensor 9111, while occlusions can increase force at sensor 9111. In other embodiments of the present invention, an electrical contact can be included on the surface of diaphragm 9101 and can create an electrical switch when contact is made between actuator 9108 and diaphragm 9101. The electrical switch can be used to verify motion of actuator 9108.

As mentioned previously, a reservoir is typically connected to inlet channel 9103. An error mode can occur if the pressure in the reservoir is suddenly increased to unusually high pressures while actuator 9108 is in the up position. If the pressure in the reservoir is high enough, infusion liquid will overcome the backpressure of inlet check valve 9106 and outlet check valve 9104, causing flow through the pump, even when it is off. To overcome this error, some embodiments of the present invention include an over-pressure check valve. Over-pressure check valve is oriented in an opposite direction to inlet check valve 9106. Over-pressure check valve allows infusion liquid to pass when the reservoir is at normal pressure but closes when the reservoir is at unusually high pressure. The pressure required to close over-pressure check valve is greater than the pressure encountered during normal operation, when actuator 9108 is in the up position and a slight drop in pressure has been created in pump chamber 9105. If the pressure in the reservoir becomes unusually high (from an impact or from a change in airplane cabin pressure, for example), over-pressure check valve will seal, preventing inadvertent flow of infusion liquid. Over-pressure check valve can ensure that there is no delivery of infusion liquid at abnormal reservoir pressures. If over-pressure check valve seals, a drop in pressure may form in pump chamber 9105 when actuator 9108 moves up, and diaphragm 9101 will typically stay in the down position. In embodiments where an electrical contact has been included in diaphragm 9101, the electrical switch between actuator 9108 and diaphragm 9101 will stay open when diaphragm 9101 stays in the down position and actuator 9108 is up, and an alarm can be raised to alert the user. In other embodiments of the present invention, active valves, rather than check valves, are used to prevent flow from an over pressurized reservoir. Active valves rely on direct physical contact with an actuator to close, while check valves rely upon pressure differential across the valve to close. Active valves are typically more complicated than check valves, however, and in some cases require more sophisticated actuation.

Micro diaphragm pumps, according to the present invention, are a type of positive displacement pump. In positive displacement pumps, a pump chamber is filled then emptied by action of the pump. A distinct advantage of micro diaphragm pumps (and positive displacement pumps, in general) is that they can pump gas as well as liquid, if the compression ratio is high enough. The compression ratio is the volume displaced during the actuator down stroke divided by the volume of the pump chamber. Using a micro diaphragm pump is particularly advantageous when priming the pump, since air is expelled from the pump (and its inlet and outline lines) during priming. Micro diaphragm pumps are easy for a user to set up because they can pump air and infusion liquid. Centrifugal pumps, on the other hand, rely upon shear between an impeller and the liquid being pumped. Centrifugal pumps work better with liquid than with air and are more difficult to set up.

As mentioned previously, a variety of methods can be used to fabricate micro diaphragm pumps, according to the present invention. Thin polymer and metal films can be laminated together to form a micro diaphragm pump. Layers of thermally activated adhesives can be used to laminate the films together. Check valves can include springs made from metal or plastic sheets. Check valve springs can be biased to create particular cracking and sealing pressure. Bias can be varied by controlling the relative position of the check valve and the surface against which it seats. Check valve springs can be made by chemically etching metal sheet or foil, or by cutting or injection molding plastics. Pump chamber volume can be established by the thickness of the metal and/or polymer and adhesive films. If necessary, the wetted surfaces of the pump can be coated with a polymer (such as parylene), to improve compatibility with infusion liquids. Ultrasonic welding, or other bonding methods, can be used instead of, or in addition to, thermally activated adhesives. Compatibility with the infusion liquid is a particularly important requirement of micro diaphragm pumps of the present invention. In many embodiments, the infusion liquid is in direct contact with many parts of the pump. Infusion liquid can stick to wetted pump surfaces, and can be modified by chemical and/or physical interaction. In some embodiments of the present invention, wetted pump components are made out of biocompatible materials, such as polypropylene. In other embodiments, wetted pump components are coated with biocompatible materials such as paralyne, PEG, PAA, PVP, and/or polyelectrolyte. Biocompatible materials minimize adsorption of infusion liquid, and its degradation. Alternatively, pump components can be machined or injection molded using biocompatible polymers, such as PMMA, polycarbonate, polycyclic olefin, polystyrene, polyethylene, or polypropylene.

### 4. Valveless Micro Pump

A valveless MEMS micropump capable of improved efficiency and performance is disclosed. The micropump includes two adjoining chambers separated by a piezoelectric actuated pump membrane. The micropump moves fluid through the chambers through diffuser elements characterized by differential directional resistance to fluid flow by piezoelectric actuation of the pump membrane.

Referring now to the drawings, FIG. 9N (A) which show an embodiment of a micropump 9120 according to principles of the present invention. The micropump 9120 includes two chambers, an upper chamber 9121 and a lower chamber 9122, separated by a common pump membrane 9123. Each chamber has at least two diffuser elements 9124 for permitting fluid flow into and out of each chamber. The diffuser elements 9124 each have a chamber end 9125 opening to the interior of upper chamber 9121 or lower chamber 9122 and an exterior end 9126 opening out of the micropump 9120. A variety of geometries can be employed for the diffuser element 9124. Several examples of such geometries, specifically a conical diffuser and two types of flat walled diffusers. While a conical geometry is acceptable, flat walled diffusers are preferred since they provide better performance in a more compact design. Preferably, a four-sided frusto-pyramidal diffuser element is used for ease of manufacturing and enhanced performance. It should be understood that geometries other than specified herein may be used for the diffuser elements 9124. If desired, curved wall sections may also be used for the diffuser, although this has not been found to be necessary. The choice of diffuser geometry may also be dependent on the fabrication process used. The dimensions of the diffuser elements depend on the properties of the fluid to be pumped and on the desired optimum working frequency and force of which the fluid is to be pumped.

The chambers preferably have identical dimensions with a simple geometry. Preferably the chambers are made of a single wafer of silicon to provide additional structural stiffness and eliminate the need for junctions. The chambers can be machined using a variety of physical and chemical etching techniques such as wet etching, dry etching, or deep reactive ion etching.

The flexible membrane 9123 is a layered composite of a number of materials forming a common partition separating the upper chamber 9121 and the lower chamber 9122. In addition, the membrane 9123 acts as a diaphragm under the appropriate stimuli, flexing to increase or decrease the volume within the upper chamber 9121 and the lower chamber 9122. The membrane is designed to minimize stress concentration points in order to permit operation under high stress and at high frequency. Layers can be permanently joined using wafer bonding techniques such as fusion bonding, anodic bonding, and eutectic bonding.

The composition of the pump membrane 9123 in a preferred embodiment actuated by piezoelectricity. As shown in FIG. 9N (B) a passive intermediate layer 9127 is designed to provide structural support for the pump membrane 9123. The material chosen for the intermediate layer 9123 should be stiff enough to support the stresses applied by the fluid being cycled through the micropump 9100 while permitting repeated piezoelectric driven deformation. The material for intermediate layer 9127 should also be chosen so that the stiffness of the intermediate layer is similar to that of the piezoelectric material to ensure a homogenous stress distribution over the intermediate layer 9127 when the piezoelectric material is deformed. Intermediate layer 9127 is preferably composed of PYREX® 7740 material, but it should be understood that suitable replacements can be chosen. The intermediate layer 9127 is disposed between two piezoelectric discs 9128. A piezoelectric disc 9128 is formed by stratifying a layer of piezoelectric material between two layers of conducting material. Piezoelectric material is made with Piezo Material Lead Zirconate Titanate (PZT-5A), although other piezoelectric materials can be used. The conducting material may be composed of an epoxy such as the commercially available EPO-TEK H31 epoxy. The epoxy serves as a glue and a conductor to transmit power to the piezoelectric discs 9128. The piezoelectric discs 9128 are secured to the surface of the intermediate layer 9127, so that when a voltage is applied to the membrane 9100, a moment is formed to cause the membrane 9123 to deform.

The layered pump membrane 9123 further includes a nonconducting cover 9129 covering both faces of the membrane 9123. The covers 9129 are composed of an electrically insulating material such as silicone rubber. The cover 9129 serves to insulate the piezoelectric discs 9128 from the fluid being pumped as well as to create a gasket to seal the chambers 9121 and 9122 from fluid leakage and communication with each other.

The pump membrane 9100 thus comprises piezoelectric, conducting and insulating materials. The choice of materials depends on considerations including the need for increased chemical resistance to the fluid being transported, and the adjustment of electrical resistance and physical properties such as elasticity of the pump membrane. Ideally, the chosen materials are flexible in a range sufficient to permit piezoelectric activity to actuate the pump, are chemically inert to the fluid being transported and are physically resistant to stresses that would occur over the desired life cycle of the micropump.

The operation of the micropump 9100 will now be described with reference to FIG. 9O (A)-(C). At rest, the upper chamber 9121 and the lower chamber 9122 are separated by a diaphragm pump membrane 9123 as shown in FIG. 9O (A). A pair of diffuser elements 9124 are in fluid communication with each chamber. Diffuser elements 9124 are oriented so that the larger cross-sectional area end of one diffuser element is opposite the smaller cross-sectional area end of the diffuser element on the other side of the chamber. This permits a net pumping action across the chamber when the membrane is deformed.

The piezoelectric discs are attached to both the bottom and the top of the membrane. Piezoelectric deformation of the plates is varied by varying the applied voltage so as to excite the membrane with different frequency modes. Piezoelectric deformation of the cooperating plates puts the membrane into motion. Adjustments are made to the applied voltage and, if necessary, the choice of piezoelectric material, so as to optimize the rate of membrane actuation as well as the flow rate. Application of an electrical voltage induces a mechanical stress within the piezoelectric material in the pump membrane 9123 in a known manner. The deformation of the pump membrane 9123 changes the internal volume of upper chamber 9121 and lower chamber 9122 as shown in FIG. 9O (B). As the volume of the upper chamber 9121 decreases, pressure increases in the upper chamber 9121 relative to the rest state. During this contraction mode, the overpressure in the chamber causes fluid to flow out the upper chamber 9121 through diffuser elements 9124 on both sides of the chamber. However, owing to the geometry of the tapered diffuser elements, specifically the smaller cross-sectional area in the chamber end of the left diffuser element relative to the larger cross-sectional area of the right diffuser element, fluid flow out of the left diffuser element is greater than the fluid flow out the right diffuser element. This disparity results in a net pumping of fluid flowing out of the chamber to the left.

At the same time, the volume of the lower chamber 9122 increases with the deformation of the pump member 9123, resulting in an under-pressure in the lower chamber 9122 relative to the rest state. During this expansion mode, fluid enters the lower chamber 9122 from both the left and the right diffuser elements 9124. Again owing to the relative cross-sectional geometry of the tapered diffuser elements, fluid flow into the lower chamber 9122 through the right diffuser element is greater than the fluid drawn into the lower chamber 9122 through the left diffuser element. This results in a net fluid flow through the right diffuser element into the chamber, priming the chamber for the pump cycle.

Deflection of the membrane 9123 in the opposite direction produces the opposite response for each chamber. As shown in FIG. 9O (C), the volume of the upper chamber 9122 is increased. Now in expansion mode, fluid flows into the chamber from both the left and right sides, but the fluid flow from the right diffuser element is greater than the fluid flow from the left diffuser element. This results in a net intake of fluid from the right diffuser element, priming the upper chamber 9122 for the pump cycle. Conversely, the lower chamber 9122 is now in contraction mode, expelling a greater fluid flow from the lower chamber 9122 through the left diffuser element than the right diffuser element. The result is a net fluid flow out of the lower chamber 9122 to the left.

Micropumps are small-scale devices that generate fluid flow at micro- or nanoliter per minute levels, and they find applications in various fields such as drug delivery systems, lab-on-a-chip devices, and microfluidic systems. In an embodiment the micro-pump may be at least one of a piezoelectric micro-pump, an electroosmotic micro-pump, a peristaltic micro-pump, a thermal micro-pump, a shape memory alloy micro-pump, a valveless micro-pump, a syringe micro-pump, an electrochemical micro-pump, a magnetic micro-pump, an electromagnetic micro-pump, a centrifugal micro-pump, a hydraulic micro-pump, a micro-blower micro-pump, and an electrospray micro-pump. Each type of micropump has its unique mechanism for fluid flow generation, and the choice of micropump depends on specific requirements such as flow rate, fluid type, and power consumption. Each type of micropump uses a unique mechanism to generate fluid flow, and their design and selection depend on the specific requirements of the drug delivery. While certain micro-pumps have been described in detail, the applicant contemplates the use of any micro-pump mentioned in this application and those from documents incorporated by reference.

As can be seen from FIG. 9O (A)-(C), one frequency cycle of the membrane 9123 causes the upper chamber 9121 and the lower chamber 9122 to alternately supply and pump fluid in the right to left direction. It will be readily apparent that the two chambers do not need to pump fluid in the same direction. The direction of fluid flow for one chamber can be reversed independently of the other chamber simply by reversing the configuration of the diffuser elements serving the particular chamber of interest.

Performance of the double superimposed chamber micropump is superior to a single chamber micropump. By optimizing geometric characteristics of the chamber and diffuser elements for the mechanical properties of the fluid to be pumped, net flow rates are significantly improved relative to a single chambered micropump with equivalent geometric dimensions in a low frequency field. Moreover, the double chambered micropump operates at a lower or equal membrane displacement and improves the maximum net flow frequency compared to a single chambered micropump.

Micropumps according to principles of the present invention may be operated at a substantially lower maximum flow working frequency. This results in savings in power consumption requirements and improves overall pump efficiency. Micropumps according to principles of the present invention can be constructed using well-known MEMS techniques and materials, providing a further economic advantage.

Micropumps according to principles of the present invention provide a readily available technology for crucial applications, including life support and ongoing critical medical care. Micropumps according to principles of the present invention overcome real world problems, increasing pump efficiency despite fluid leakage losses (i.e. the micropumps exhibit improved volume metric efficiency), frictional losses (i.e. they exhibit improved mechanical efficiency) and losses due to imperfect pump construction (i.e. the micropumps exhibit improved hydraulic efficiency). Further, micropumps according to principles of the present invention can be employed to deliver a wide variety of materials in gaseous, liquid, or mixed phases. By avoiding the presence of movable parts such as check valves, inherent reliability, otherwise compromised by wear and fatigue, is substantially increased. Also, pressure loss and clogging of the working fluid, especially particle-ladened fluids, at one or more check valves is also avoided.

As mentioned, micropumps according to principles of the present invention are suitable for use in critical applications requiring equipment to be highly miniaturized. In one example, a micropump according to principles of the present invention, and of the type illustrated in the Figures, has a chamber side at length of 10 mm, and a chamber height equal to the nozzles/diffuser final width. The nozzles/diffusers have a length of 1.5 mm, an initial width of 150 μm and an opening angle of 5 degrees.

Compared to single chambered designs, micropumps according to principles of the present invention have a maximum flow working frequency that is about 30% lower than the single chambered design, with the same applied force on the membrane and the same geometry and materials. Further, micropumps according to principles of the present invention have a maximum flow rate that is 40% greater than that of comparable single chamber pumps. With the application of lower operating frequencies, micropumps according to principles of the present invention exhibit a 120% improvement in maximum flow rate.

It should be understood that while the operation of the preferred embodiments above has been described for actuating the pump through piezoelectric means, other actuation means such as thermos-pneumatic, electrostatic, pneumatic or other actuation means can be readily substituted.

In an embodiment, the valveless micropump discussed herein to maintain differential pressure created by flow of gas in or out of a pump compartment may also obviate the need for nozzles and diffusers. Such mechanisms may be referenced from US20210363983A1, titled “Micro Pump Systems and Processing Techniques” which is incorporated herein in entirety.

The inlet holes allowing interstitial fluid to enter the device may be filtered. Additionally, the filters may be made from or coated with a material known to prevent or reduce clotting or buildup of proteins.

In some embodiments, the device may comprise a valve. The pharmaceutical fluid exits from the valve output via the drug delivery orifice to the body.

Additionally, if the desire is to increase the initial release of pharmaceutical fluid into the body such as when doses will be relatively large, methods may be employed to include: 1) increasing the solute to increase the osmotic pressure and 2) changing the compressibility of internal components in order to store more fluid energy, or 3) adding a component with high compressibility to the internal components subject to osmotic pressure, for example, an air-filled bladder to the piston or the use of compressible seals to act as a pressure reserve or pressure accumulator.

The pressure accumulator may be any closed cell compressible volume that compresses under pressure and releases the compressed energy as the pressure falls, could be used. This includes a gas filled bladder, a flexible foam filled volume, or any other type of pressure accumulator known to the industry. The pressure accumulator could be placed in any location that is in communication with the osmotic chamber or the pharmaceutical chamber. For example, within the hollow cavity of the piston (FIG. 9P) or as a disk within the osmotic chamber (FIG. 9Q).

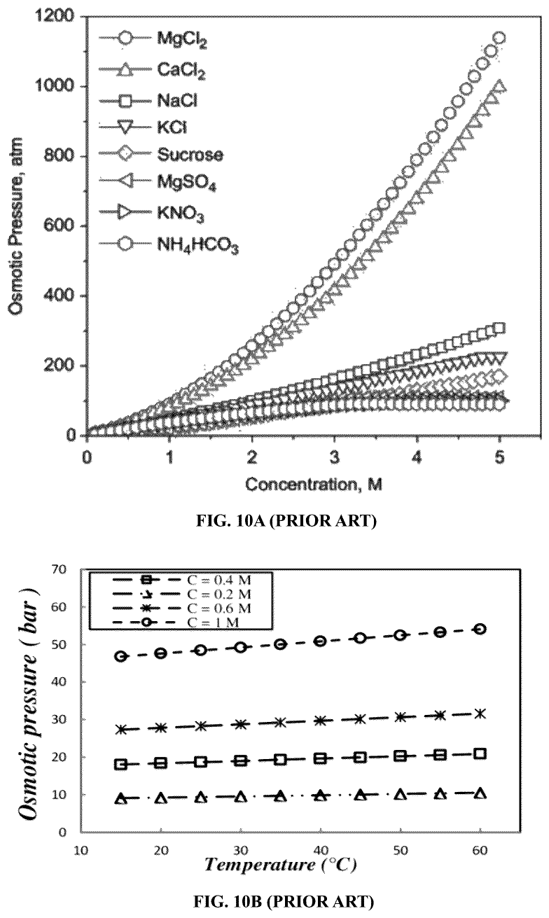

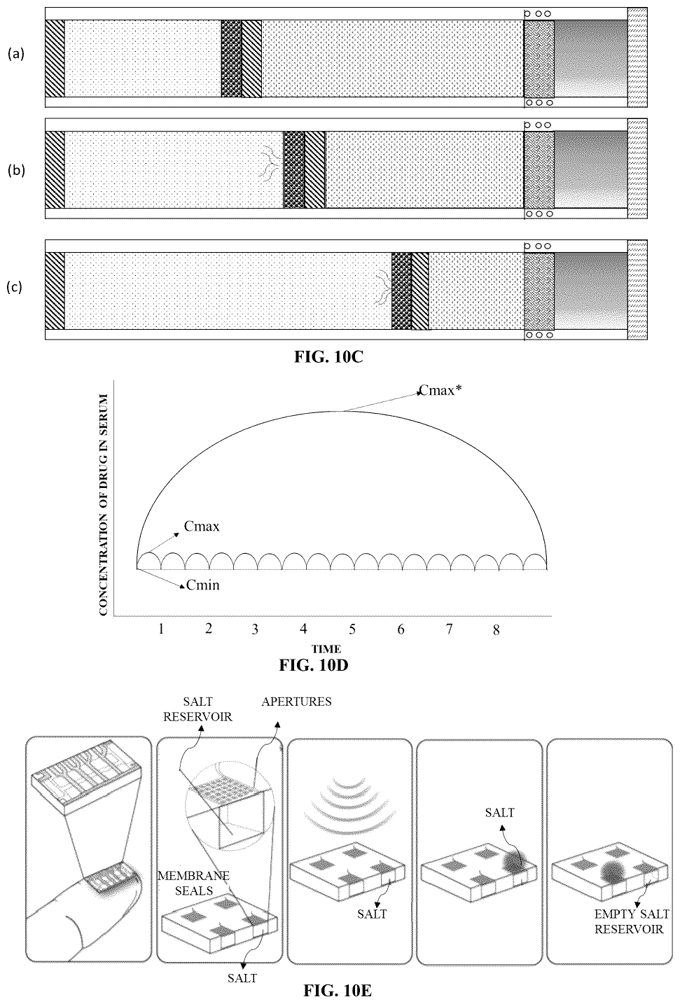

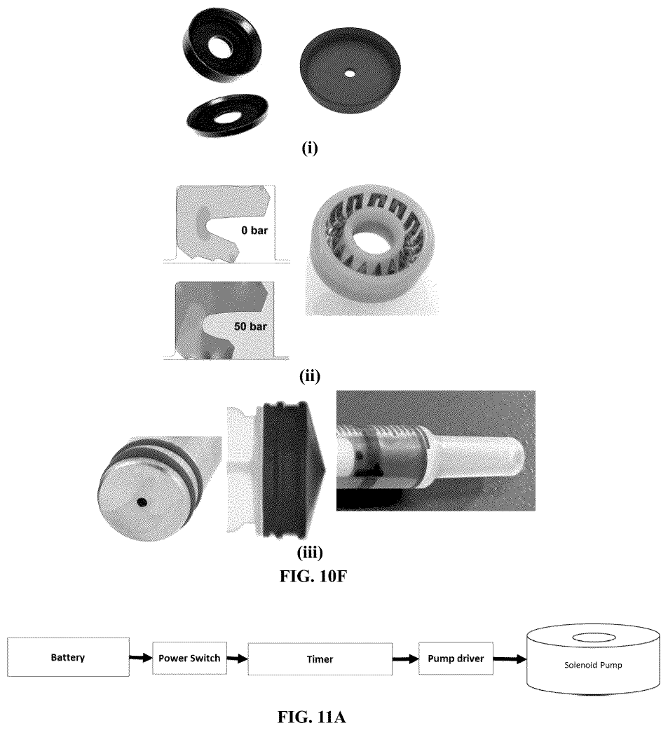

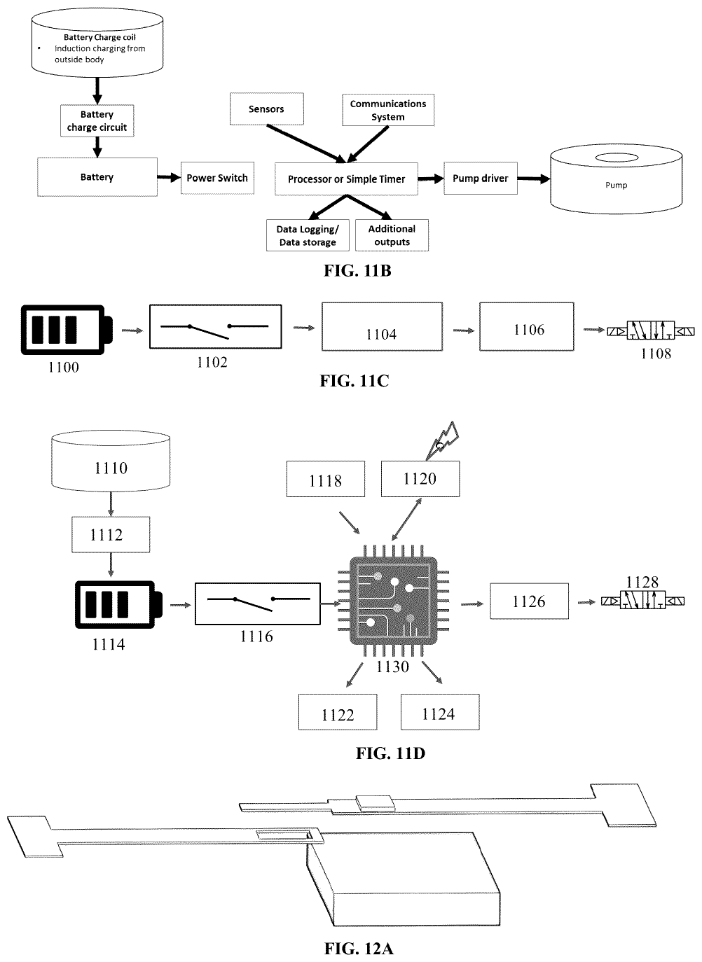

Referring to FIG. 10A, it is a graphical representation of osmotic pressure exerted by various solutes at various concentrations. In some embodiments, the osmotic agent should be capable of generating osmotic pressure between 0 and 5200 psi. In some embodiments, the osmotic agent should be capable of generating osmotic pressure between 0 and 10000 psi. Sodium chloride (NaCl) with appropriate tableting agents (lubricants and binders, e.g., cellulosic and povidone binders) and viscosity modifying agents, such as sodium carboxymethylcellulose or sodium polyacrylate are examples of preferred osmotic agents. Other osmotic agents useful as the water-swellable agent include osmopolymers and osmotic agents and are described, for example, in U.S. Pat. No. 5,413,572. A liquid or gel additive or filler may be added to the chamber containing the osmotic agent formulation to exclude air spaces. Exclusion of air from the devices generally means that delivery rates will be less affected by nominal external pressure changes. The osmotic agent may be manufactured by a variety of techniques, many of which are known in the art (see, e.g., U.S. Pat. Nos. 6,923,800 and 6,287,295). In one such technique, an osmotically active agent is prepared as solid or semi-solid formulations and pressed into pellets or tablets whose dimensions correspond to slightly less than the internal dimensions of the respective chambers that they will occupy in the enclosure interior. Depending on the nature of the materials used, the agent and other solid ingredients, that may be included, may be processed prior to the formation of the pellets by such procedures as ball-milling, calendaring, stirring, or roll-milling to achieve a fine particle size with fairly uniform mixtures of each ingredient. The enclosure for pressing the osmotic agent into tablets or pellets may be formed from a wall-forming material by the use of a mold, with the materials applied either over the mold or inside the mold, depending on the mold configuration.

Referring to FIG. 10B, it is a graphical representation of osmotic pressure exerted by sodium chloride salt at four concentrations at various temperatures. The osmotic pressure exerted by the osmotic solution depends on many parameters such as concentration and temperature. For a selected membrane material, solute, and drug concentration, and thickness L, the liquid permeation rate dV/dt through the membrane is directly proportional to the liquid surface area of the membrane body. The solute is selected such that it provides minimum variability in osmotic pressure due to slight concentration or temperature change.

Referring to FIG. 10C, it shows a movement of a piston assembly driven by osmotic pressure, according to one or more embodiments. When the osmotic pressure exerted by the osmotic unit on the piston assembly is above a predefined minimum pressure, there is no solute diffusion via the chip (depicted in (a) and (c) of FIG. 10C). However, when the pressure falls to the predefined minimum pressure, there is diffusion of solute into the osmotic unit till there is an increase in the osmotic pressure to a predefined maximum pressure (depicted in (b) of FIG. 10C).

FIG. 10D shows a schematic to maintain a substantially constant drug concentration profile below the concentration of the drug at which the risk of side effects is high. Cₘᵢₙ is the minimum concentration of the drug, below which the drug may not be effective at controlling the target condition. C_{max*} is the concentration of drug above which the risk of side effects increases. Cₘₐₓ is a concentration between Cₘᵢₙ and Cₘₐₓ that provides an effective and tolerable therapy. The device is configured to maintain the concentration at Cₘₐₓ. If the concentration is above Cₘₐₓ the drug flow is stopped and if the concentration goes below a Cₘᵢₙ, the drug flow is allowed. The concentration of the drug is maintained between a concentration range of Cₘᵢₙ and Cₘₐₓ in the body of the subject, thereby minimizing adverse effects or toxicity. It also aids in selecting patients for clinical trials, identifying, and excluding those at risk early on, thus enhancing trial integrity and accelerating medical innovation.

An embodiment relates to repurposing failed drugs through the device. This not only speeds up the availability of treatments but also reduces the costs and challenges of bringing new drugs to market. The repurposing process maintains safety and efficacy, ensuring only compounds with proven safety profiles are considered.

Referring to FIG. 10E, it is a schematic showing a chip comprising a solute reservoir assembly, according to one or more embodiments. The device may comprise a chip comprising a solute reservoir assembly, according to one or more embodiments. The chip can be programmed to release the osmotic agent into the osmotic solution when pressure drops below a pre-defined limit. The chip may comprise an array of micro sized solute reservoirs that can be covered by a membrane seal. The membrane seal can be made up of a phase change material made as donut shaped disks or rectangular mats overlaid on the inside surface of the solute reservoir assembly. In an example, when electrical current is applied to the chip, it will get activated and the membrane seal will melt open an aperture in the solute reservoirs. This will allow release of the osmotic agent into the osmotic unit via the aperture. Thus, the solute concentration will increase, thereby increasing the osmotic pressure above the pre-defined limit. When the solute reservoir is empty, the next dose of solute can be released when the osmotic pressure reaches again below the pre-defined limit.

### I. Seals

In an embodiment, seals have three main functions: 1) Sealing elements must conform closely enough to the microscopic irregularities of the mating surfaces (rod to seal groove and/or piston groove to cylinder bore, for example) to prevent pressure fluid penetration or passage; 2) The seal must have sufficient resilience to adjust to changes in the distance between mating surfaces during a cylinder stroke. This clearance gap changes size because of variations in the roundness and diameter of the cylinder parts. The clearance gap also may change size in response to side loads. As the size of the gap changes, the seal must match the size change to maintain compressive sealing force against adjacent mating surfaces; 3) seal must resist shear forces that result from the pressure differential between the pressurized and unpressurized sides of the seal. The seal must have sufficient strength and stiffness to resist becoming deformed into the gap and damaged or destroyed.

### 1.1 Mechanical Seal

In a mechanical seal serving as an example of a sliding part, while maintaining a sealing property, sliding friction during rotation is required to be reduced to the extreme. By variously texturing the sliding faces, a method of reducing friction is realized. In an embodiment, texturing could be the arrangement of dimples on the sliding faces. In order to reduce a friction coefficient of the sliding face, it is desirable to activate in a fluid lubricating state. Due to the dent shape of the dimples, a fluid lubricating operation is obtained, as described in U.S. Ser. No. 11/035,411B2.

In a mechanical seal, the pressure in the chamber may not be instantaneously eliminated at the beginning of the return stroke of the piston, therefore in an embodiment, it may be desirable to maintain a pressure differential during this latter period also.

In an embodiment, a seal assembly may be as described in U.S. Pat. No. 3,544,118A. A seal assembly comprising a series of axially spaced seal cups each having a cavity for retaining a seal ring in contact with a rod passing through the cup; each of the cups being connected by a gas passage of a size and shape to act as an expansion reservoir; each seal ring including gas ports connecting one side of the ring with the other; said expansion reservoirs and gas ports constructed in definite proportions to provide a leakage path with certain desired characteristics as hereinafter set forth. The characteristics of the leakage path are such that a finite pressure drop is achieved across each ring during most of the compression and return stroke of the rod.

### 1.2 Annular Seal

In an embodiment, seals used herein may be annular seal assemblies. More than one type of seal may be used on the same piston. A leakage of fluid across the face of a sliding seals varying degrees of compressive creep or flow. Expanded polytetrafluoroethylene (ePTFE) is of a higher strength than conventional PTFE, has the chemical inertness of conventional PTFE, and has an increased temperature range of up to 315° C.

### 1.3 Seal Material

In an embodiment, a seal could be made of PTFE, filled PTFE®, engineered thermoplastics, high-performance polyurethanes, NBR (Nitrile), FKM, Viton®, HNBR, EPDM, Aflas®, Hytrel®, Silicone, low- and high-temperature, FDA-compliant seal material grades and proprietary specialized Duralast™, Duraloy™, and Permachem™ materials.

In an embodiment, seal material may be comprising a cylindrical core of an elongated polytetrafluoroethylene (PTFE) contained within a tight wrap of high strength film. The core of elongated polytetrafluoroethylene (PTFE) formed into a loop; means to constrain the core from lateral flow when the core is placed under compressive pressure to establish and maintain a fluid seal, as disclosed in U.S. Pat. No. 5,486,010A, incorporated herein by reference in entirety. In an embodiment, seal material may be comprising a cylindrical core of a porous expanded PTFE contained within a tight wrap of high strength film.

In another embodiment, a seal could be made of a rubber with a coating of PTFE tapes to provide it chemical resistance. The PTFE could be uniaxially, biaxially or multiaxially expanded PTFE tapes, or combinations thereof.

### 1.3.1 Expanded PTFE Seal

In an embodiment, Expanded PTFE may be coated to provide properties such as resilience, electrochemical responsiveness, added strength, further reduced creep relaxation, and the like.

Porous ePTFE joint sealants have proven to have excellent seals in many applications. In an embodiment, expanded PTFE core may be wrapped by a tape of porous ePTFE.

### 1.3.2 Filled PTFE

In an embodiment, filled PTFE may contain a particulate filler. The term “particulate” as used herein is meant to include particles of any aspect ratio and thus includes particles, chopped fibers, whiskers, and the like. The particulate filler may be an inorganic filler which includes metals, semi-metals, metal oxides, carbon, graphite, and glass. Alternatively, the particulate filler may be an organic filler, which includes polymeric resins. Suitable resins include, for example, polyether ether ketone (PEEK), fluorinated ethylene propylene (FEP), copolymer of tetrafluoroethylene and perfluoro(propylvinyl ether)(PFA), and other similar high melting polymers. Particulate fillers, when used, are selected to impart or enhance certain properties in the core or wrapping film according to the application in which the composite gasket material of the invention will be used. For example, particulate fillers can also be used to modify compressibility and dimensional stability properties of the composite gasket material.

### 1.4 Biocompatibility of Seal

In some embodiments, the seal is biocompatible material. In some embodiments, the biocompatible material is silicone. In some embodiments, the biocompatible material is PTFE. In some embodiments, the biocompatible material is polyurethane. In some embodiments, the biocompatible material is epoxy resins. In some embodiments, the biocompatible material is hydrogel. In some embodiments, the biocompatible material is olefin.

Referring to FIG. 10F, it illustrates sample seals for the device, according to one or more embodiments. The non-limiting examples of the seal are A) Cone seal, B) Fluid pressure assisted seal, C) Spring assisted seal, and D) O-ring or ridge seals. In an example the cone seal is made of Teflon or Nylon. Fluid pressure assisted seals use the pressure of the fluid during movement to enhance the seal. Spring assisted seal comprises using a spring to enhance the seal. O-ring or ridge seals are elastomer seals that rely on material compression to seal. The seal may be one of many types as shown in FIG. 10F. The most common seal type is the O-ring or ridges, like that used in most syringes. Other types may be chosen to reduce either the sliding (dynamic) forces or to reduce an initial (startup) force. Seal material should be chosen considering both of these forces. Teflon (PTFE) has low coefficients of friction for both static and dynamic sealing.

Seals described in U.S. Pat. Nos. 7,658,387B2, 7,179,525B2, 5,492,336A, 3,544,118A, U.S. Ser. Nos. 11/035,411B2, 10/876,634B2, U.S. Pat. No. 5,486,010A, are incorporated herein by reference in their entirety.

In an embodiment, the drug delivery unit is fluidically connected to the body fluid of the subject. This allows the drug to get mixed into the body fluid to perform pharmaceutical action.

### J. Electronics

In an embodiment, the PCB controls the closing and opening of the valve. In another embodiment, PCB uses a coil present within the valve to control the valve. In another embodiment, the PCB communicates with external devices via an inductive communication method. In an example, radio frequency can be used for communicating with external devices. In another example, infra-red can be used for communicating with external devices.

FIG. 11A illustrates an electronic unit of an implantable device, according to one or more embodiments. The electronic unit is adapted to supply power to switch on or switch off the implantable device. The electronic unit comprises a battery, a power switch, a timer, and a solenoid. The battery may be a rechargeable battery and/or a non-rechargeable battery. The battery may be recharged while the implantable device remains implanted. In an example, the battery may comprise of one of a lithium iodine battery, a lithium manganese dioxide battery, a lithium carbon monofluoride battery, a lithium carbon monofluoride and silver vanadium oxide (Li/CFₓ—SVO) hybrid batteries, a lithium silver vanadium oxide battery, a lithium-ion battery. The power switch is adapted to trigger/actuate the implantable device. The power switch is a switch that maintains its state after being activated. Whenever the power switch is actuated, whichever state the switch is left in, it will persist until the switch is actuated again. For example, the power switch, once actuated, remains in ‘ON’ state permanently. A push-to-make, push-to-break switch may be a latching switch. The power switch may be actuated using the magnetic field. In an embodiment, the power switch is an electromechanical switch operated by an applied magnetic field. The power switch may be a reed switch. In an embodiment, the power switch may be actuated by other means (e.g., mechanical, electrical, etc.). The power switch may also be a latch switch. The timer is configured to actuate the implantable device for a predefined time period. The predefined time period may be set or adjusted as per the requirement. The timer gets activated upon activation by the power switch. The timer actuates the solenoid for the predefined time to infuse medication into a patient's body. The timer is adapted to actuate the implantable device for the predefined amount of time to provide the medication at a predefined dose.

The electronic unit may also comprise a pump driver. The pump driver is used to drive the solenoid. The implantable device may be driven by the pump driver during normal operation but may also operate using the battery. In an embodiment, the pump driver comprises a driving element that is coupled to the solenoid. The driving element comprises a means for generating magnetic fields. The magnetic field may be utilized to actuate the solenoid. In another embodiment, the pump driver may also be located outside a patient's body. The pump driver may drive the solenoid from a location outside the patient body. The pump driver is useful during emergency situations to directly drive the solenoid if the battery fails or if the internal coils fail.

The solenoid consists of a helix of wire wrapped around an iron or steel core. The core becomes magnetized when electrical current passes through the coil. The solenoid, when actuated, moves against a spring to slide a diaphragm of the valve into a discharge position. When electric current is removed, the diaphragm slides back into the suction position. The solenoid is designed so the electromagnet cannot move the diaphragm against a resistant pressure of flowing medium (gas or liquid) (backpressure) that would cause it to fail. The solenoid can pump against a dead head, or infinite backpressure, when discharge is closed off. The dead head is a situation where the discharge of the pump is obstructed to the point that there is zero flow.

FIG. 11B illustrates an electronic control unit of an implantable device, according to one or more embodiments. The electronic control unit comprises a battery charge coil, a battery charge circuit, a battery, a power switch, a processor, a pump driver, and a pump. The battery charging coil comprises a charging coil configured to selectively provide a field to provide power to the battery charging circuit of the implantable device. The battery charging coil works based on induction charging from the outside body. The battery charge circuit receives power from the battery charging coil and charges the battery. The battery may also receive power from alternate power sources. The alternate power sources may include energy harvesting from within the body such as body motion, thermal energy, heartbeat pulse, and body energy and metabolism. The battery may also receive power from induction current power from outside the body.

The processor is configured to perform dosing adjustments based on sensor inputs. The processor is configured to perform treatment adjustments based on sensor inputs. The processor is configured to transmit faults, out of range sensor data, low battery, etc. The processor comprises a watchdog timer to detect and recover from malfunctions and/or faults. The processor enables ultralow power standby between tasks.

The processor is further configured to perform data storage and data logging such as for dosing history, dosing adjustments, fault conditions, sensor data, battery/power condition, medical history, etc. The processor may perform data storage and data logging in a certain frequency (i.e., predefined time period). The processor is further configured to provide other outputs such as electromagnetic fields. The electromagnetic fields may be used for healing, etc. The processor is configured to control additional pumps, electric valves, etc. The pump driver is described above in detail.

The pump may be a solenoid. In an embodiment, the pump may be a diaphragm pump. In another embodiment, the pump may be a piezo pump. The pump is used to infuse the medication into the patient's body. The pump may comprise a coil assembly. The coil assembly may transmit and/or receive data and commands from external sources. The data may comprise units on confirmation, medical data dump, sensor data, etc. The coil assembly may use multiple protocols for data transmission. The coil assembly may also function as the battery charging coil by providing inductive charging power from outside the body. The solenoid is illustrated above in detail.

FIG. 11C illustrates an electronic unit of an implantable device, according to one or more embodiments. The electronic unit is adapted to supply power to switch on or switch off the implantable device. The electronic unit comprises a battery (1100), a power switch (1102), a Timer (1104), and a solenoid driver (1106) and a solenoid (1108). The battery may be a rechargeable battery and/or a non-rechargeable battery. The battery may be recharged while the implantable device remains implanted. In an example, the battery may comprise of one of a lithium iodine battery, a lithium manganese dioxide battery, a lithium carbon monofluoride battery, a lithium carbon monofluoride and silver vanadium oxide (Li/CFₓ—SVO) hybrid batteries, a lithium silver vanadium oxide battery, a lithium-ion battery. The power switch (1102) is adapted to trigger/actuate the implantable device. The power switch is a switch that maintains its state after being activated. Whenever the power switch is actuated, whichever state the switch is left in, it will persist until the switch is actuated again. For example, the power switch, once actuated, remains in ‘ON’ state permanently. A push-to-make, push-to-break switch may be a latching switch. The power switch may be actuated using the magnetic field. In an embodiment, the power switch is an electromechanical switch operated by an applied magnetic field. The power switch may be a reed switch. In an embodiment, the power switch may be actuated by other means (e.g., mechanical, electrical, etc.). The power switch may also be a latch switch.

The timer is configured to actuate the implantable device for a predefined time period. The predefined time period may be set or adjusted as per the requirement. The timer gets activated upon activation by the power switch. The timer activates the valve driver which in turn opens the valve for a certain duration to deliver a drug before closing it again. The timer is adapted to actuate the implantable device for the predefined amount of time to provide the medication at a predefined dose.

The electronic unit may also comprise a solenoid valve driver (1106). The solenoid valve driver is used to drive the solenoid valve coil(s). In an embodiment, the solenoid driver comprises a driving element that is coupled to the solenoid valve. The driving element comprises a means for generating magnetic fields. The magnetic field may be utilized to open the valve. In another embodiment, the solenoid valve driver may also be located outside a patient's body. The solenoid valve driver may drive the solenoid valve from a location outside the patient body. The solenoid valve driver is useful during emergency situations to directly drive the valve if the battery fails or if the internal coils fail.

FIG. 11D illustrates an electronic control unit of an implantable device, according to one or more embodiments. The electronic control unit comprises a battery charge coil (1110), a battery charge circuit (1112), a battery (1114), a power switch (1116), a processor (1130), a solenoid driver (1126), and a solenoid (1128). The battery charging coil (1110) comprises a charging coil configured to selectively provide a field to provide power to the battery charging circuit of the implantable device. The battery charging coil works based on induction charging from outside the body. The battery charge circuit (1112) receives power from the battery charging coil (1110) and charges the battery (1114). The battery may also receive power from alternate power sources. The alternate power sources may include energy harvesting from within the body such as body motion, thermal energy, heartbeat pulse, and body energy and metabolism. The battery may also receive power from induction current power from outside the body.

The power switch is adapted to trigger/actuate the implantable device. The power switch is a switch that maintains its state after being activated. The power switch is shown in FIG. 11C.

The processor is adapted to coordinate and control the functions of the implantable device. The processor comprises a Timer and/or Real Time Clock. The processor is configured to receive signals and/or data from sensors (1118) and communication system (1120). The sensors (1118) comprise at least one of a temperature sensor, a pH sensor, a flow sensor, a pressure sensor, an electrocardiography (ECG) sensor, a valve sensor, and a global positioning system (GPS). The communication systems (1120) may fetch information and/or data via a Bluetooth® module, Wi-Fi® module to the processor. The communication systems (1120) may also fetch information and/or data regarding inductive or magnetic pulses from battery charge coil. The communication systems may also fetch information and/or data regarding light pulses from a light sensor. The communication systems may transmit and/or receive information to and from the processor.

The processor is configured to perform dosing adjustments based on sensor inputs. The processor is configured to perform treatment adjustments based on sensor inputs. The processor is configured to transmit faults, out of range sensor data, low battery, etc. The processor comprises a watchdog timer to detect and recover from malfunctions and/or faults. The processor enables ultralow power standby between tasks.

The processor is further configured to perform data storage and data logging such as for dosing history, dosing adjustments, fault conditions, sensor data, battery/power condition, medical history, etc. The data logging unit (1122) performs the data logging and data storage operations. The processor may perform data storage and data logging in a certain frequency (i.e., predefined time period). The processor is further configured to provide other outputs (1124) such as electromagnetic fields. The electromagnetic fields may be used for healing, etc.

The coil assembly (1110) may transmit and/or receive data and commands from external sources. The data may comprise units on confirmation, medical data dump, sensor data, etc. The coil assembly may use multiple protocols for data transmission. The coil assembly may also function as the battery charging coil by providing inductive charging power from outside the body.

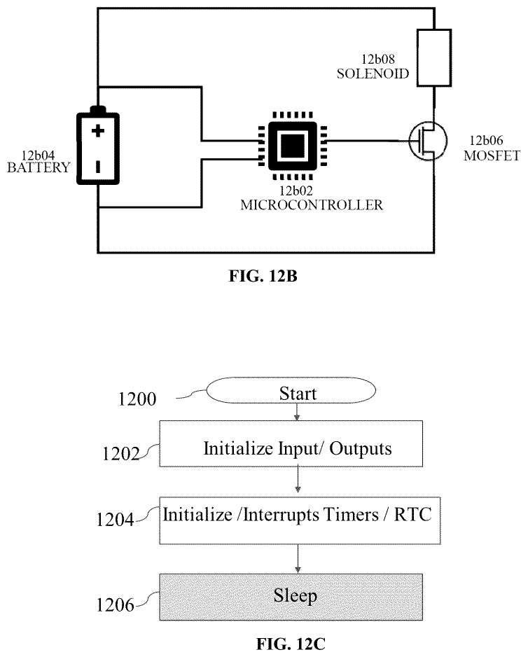

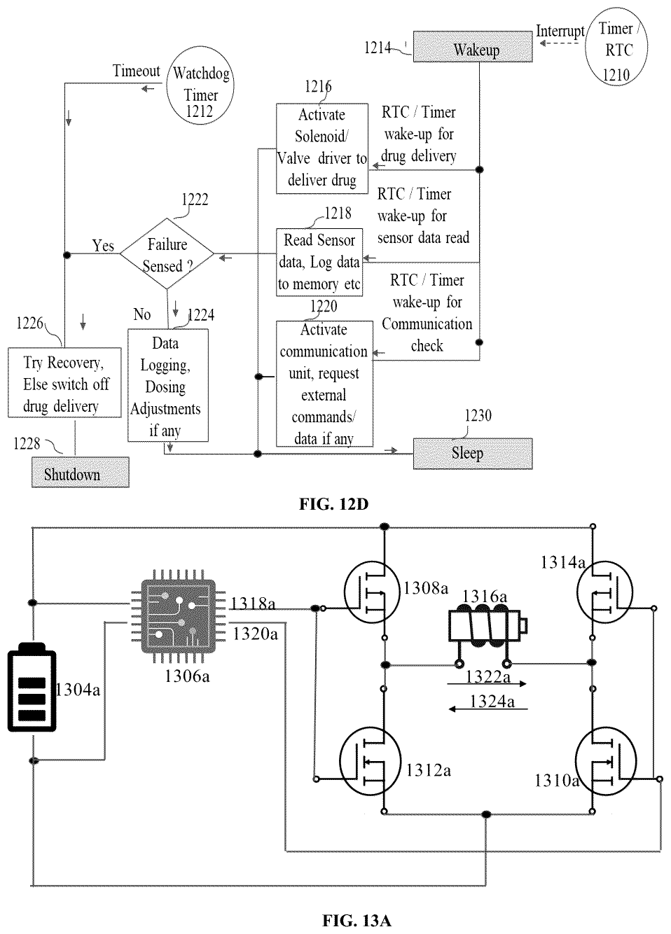

FIG. 12A illustrates a power switch, according to one or more embodiments. The power switch may be a reed switch. The power switch comprises contacts that stick when connected or actuated. The power switch comprises a first contact and a second contact. The first contact and the second contact may be made of copper. The first contact and the second contact form the power switch. The second contact comprises a material (e.g., metal) that responds to a magnet. The material may be steel or hard iron. A magnet may be placed near the power switch to place the switch in a permanent ON position or to maintain its state. When the magnet is placed in a position near the power switch, the second contact is pulled downward, contacting, and bending the first contact. The second contact tine fits into the first contact slot. When the magnet is removed, the tine remains stuck in the first contact slot continuing the electrical contact even with the magnet removed. The power switch thus remains ON permanently.

In an embodiment, the device comprises a wireless communication module that is integrated in the electronic unit of the implantable device. The wireless communication module is also integrated in a handheld telemetry wand in communication with the electronic unit of the implantable device. The wireless communication module facilitates wireless data exchange between the handheld telemetry wand and the implantable device.

In an embodiment, the device may comprise a small size microcontroller in the form of a die form or die on a flexible circuit substrate form which has a low sleep current. The microcontroller may comprise an internal timer.

Referring to FIG. 12B, it shows an embodiment of the electronics and the circuit to control the solenoid. The battery 12b04 provides power to the microcontroller 12b02. The microcontroller 12b02 sleeps for a time duration. The microcontroller 12b02 wakes up at the end of the time duration and sends a trigger to the MOSFET 12b06. The MOSFET 12b06 switches on and powers the solenoid 12b08. The battery 12b04 provides power to the solenoid 1808. In another embodiment the microcontroller 12b02 can provide power to the solenoid 12b08. A person of ordinary skill in the art will appreciate that many variations and alterations to the components and the circuit are possible.

In an embodiment, one end of the electronic unit comprises an insulation plug. In another embodiment, the insulation cap may comprise a sensor that is configured to detect a successful delivery of drug into the body of a subject. In another embodiment, the data obtained using the sensor can be used to generate a closed loop for drug delivery.

In an embodiment, the microcontroller may be programmed using a wired or wireless connection.

In an embodiment, the device comprises a wireless communication module that is integrated in the electronic unit of the implantable device. The wireless communication module is also integrated in a handheld telemetry wand in communication with the electronic unit of the implantable device. The wireless communication module facilitates wireless data exchange between the handheld telemetry wand and the implantable device.

In an embodiment, the device may comprise a small size microcontroller in the form of a die form or die on a flexible circuit substrate form which has a low sleep current. The microcontroller may comprise an internal timer.

In an embodiment the processor (microprocessor or microcontroller) within the device could be programmed to sleep most of the time once the initial initialization of the peripherals and the system Timers and/or Real Time Clock (RTC) is complete. FIG. 12C depicts this flow.

FIG. 12D illustrates the broad level program flow programmed within the processor (microcontroller or microprocessor) of the device. As depicted in the figure, processor is woken up only in two situations, either when timers and/or RTC (1210) wakes up the processor, or the watchdog timer (1212) times out. Timers/RTC within the device is/are programmed to wake up the processor a) When the timer counter reaches the predefined time of the drug delivery, b) Time to read the sensor data, and c). When communication module is to be activated to receive commands/data from the external monitoring device. Note that these three events could be programmed to take place independently and they do not necessarily occur at the same time. As an example, drug delivery could have been programmed to take place once a week, sensor reading maybe done once a day, while opening communication module could happen twice a day. Other than these three events, the device could be programmed to wake-up to perform any other critical tasks as well.

When woken up for the drug delivery, processor opens the valve for a specific duration to be able to deliver the predefined quantity of drug. A valve can be a latching type of solenoid valve which can remain open until an electrical charge of the opposite polarity is applied to close it. A small (milliseconds) pulse of electrical charge is sufficient to open or close the valve. The open state duration could be a predefined value based on the amount of dosage to be delivered. Alternatively, it could be decided by measuring the distance, ‘d’ travelled by the piston pushing the drug inside the drug chamber. Electronics circuit may include the sensor system to measure the distance by which the piston travelled to the right since the valve was opened. As soon as it completes the defined distance the valve would be closed by the Valve driver. The driver circuit is elaborated in the upcoming sections. Processor switches back to sleep mode (step 1230) post this.

When woken up for scanning sensor data (step 1218), processor reads all the connected sensors, analyses the data and stores it for further processing in the device memory. Processor may also take certain actions like adjusting the dose based on data received from the sensors. Once completed, processor will switch back to Sleep state (step 1230). If any of the sensor readings indicate pre fault or fault situation, processor takes step 1226, where attempts will be made to recover from the fault condition. Else/and processor will shut down the entire operation. The driver circuit will be disabled to stop any ongoing drug delivery. If feasible, communication module will be activated and an emergency message will be sent to the external monitoring device.

When woken-up to initiate external communication (step 1220), processor will activate the communication module, and complete any scheduled transmission of the sensor readings. During the same period processor will scan for any commands and messages from the external monitoring device. Once completed, processor will switch back to sleep mode (step 1230).

It should be noted that the predefined Timer intervals for sensor reading, and communication purposes are programmed keeping in mind the battery consumption during these steps. It's a balancing between conserving the battery life, versus the ability to timely detect the critical emergency situation.

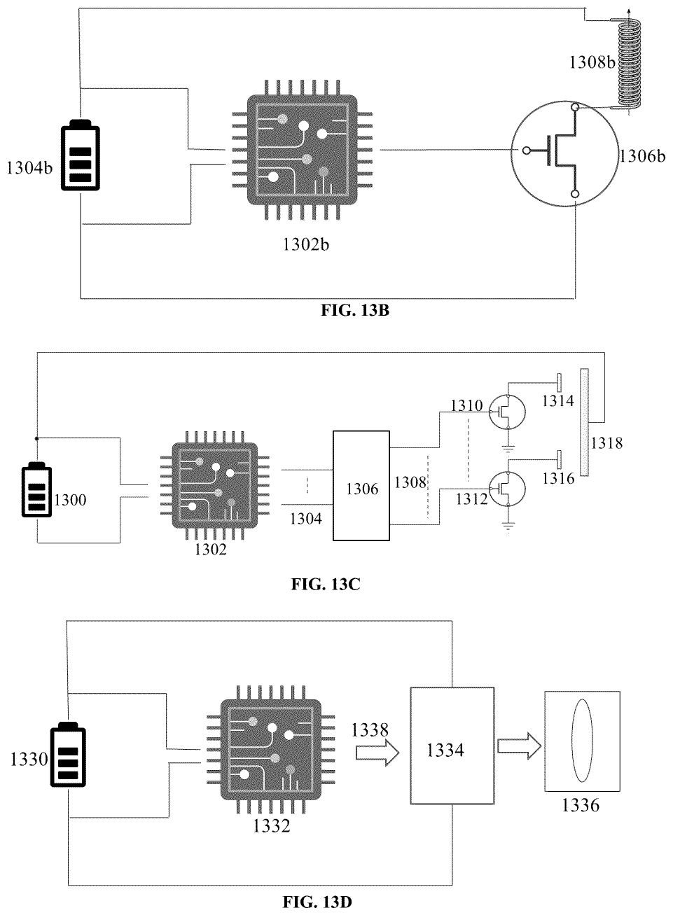

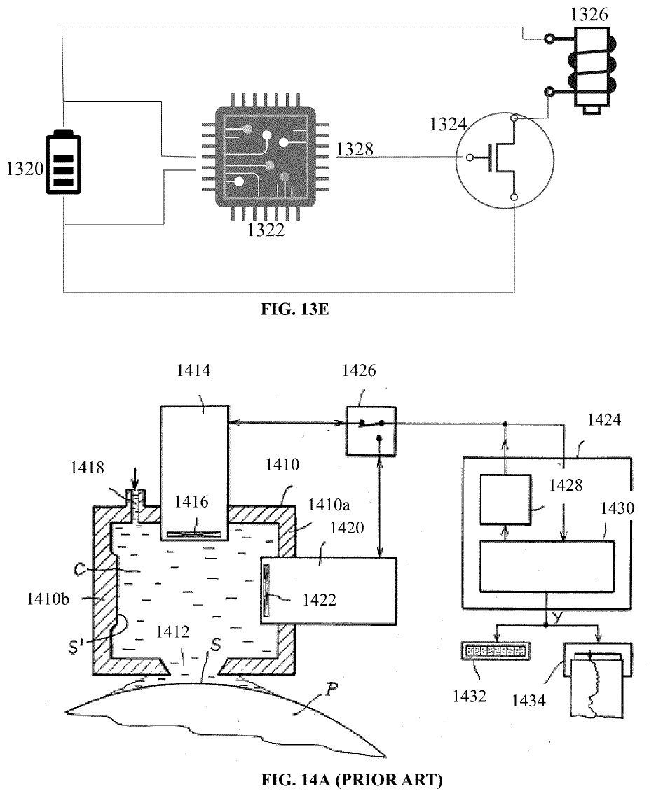

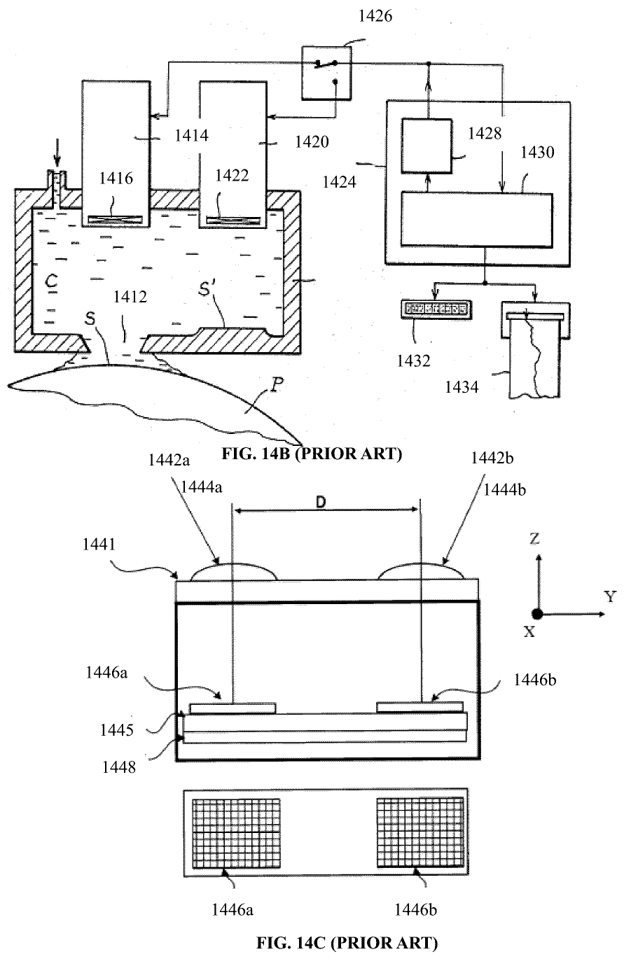

Referring to FIG. 13A, it shows an embodiment of the electronics and the driver circuit to open and close the solenoid valve. The battery 1304a provides power to the processor 1306a. The MOSFETS 1308a, 1310a, 1312a, and 1314a act like electronic switches which are controlled by the voltage level applied at their Gate lead. To open the valve, processor, using the combination of the two output ports 1318a, and 1320a, closes (switches ON) the pair of the switches 1308a and 1310a (and opens or switches off 1312a and 1314a) to establish an electrical path through the valve coil in the direction as shown by 1322a. To close the valve, processor closes (switches ON) the pair of switches 1312a, and 1314a (and opens or switches off 1308a and 1310a), to establish an electrical path through the valve coil in the direction as shown by 1324a. The electrical charge applied in both the cases will be in the form of a small pulse only. In the normal condition all the switches would be off and hence no current flows through the coil. This latching nature of the valve helps keep the battery consumption low. The power is only needed in terms of a small electrical pulse to open or close the valve. A person of ordinary skill in the art will appreciate that many variations and alterations to the components and the circuit are possible.

Referring to FIG. 13B, it shows an embodiment of the electronics and the driver circuit to control the solenoid. The battery 1304b provides power to the processor 1302b. The processor 1302b sleeps for a predefined time duration. The processor 1302b wakes up at the end of the time duration and sends a trigger to the MOSFET 1306b. The MOSFET 1306b switches on and powers the solenoid 1308b. The battery 1304b provides power to the solenoid 1308b. In another embodiment the microcontroller 1302b can provide power to the solenoid 1308b. A person of ordinary skill in the art will appreciate that many variations and alterations to the components and the circuit are possible.

Referring to FIG. 13C, it shows an embodiment of a simplistic representation of the driver circuit to control the Electrostrictive micro-pump (Peristaltic micro-pump). The electronics has been partly described under FIGS. 9E, 9F & 9G. The battery 1300 provides power to the processor 1302. During the drug delivery, the decoder 1306 will begin activating switches (MOSFETS) 1310 thro. 1312 sequentially. The switches will in turn connect the conductive panels (electrodes) 1314 thro. 1316 to the negative pole of the battery (ground) sequentially. At a time only one switch will be activated. The action is like a multiplexer that chooses conductive panels (electrodes) 1310 thro. 1312 one by one to connect to the negative pole of the battery in a sequence. The program running inside the processor (1302), using output pins 1304, sends the digital code to the decoder 1306. The decoder in turn enables the respective switch based on the digital code received on the processor output lines 1304. This multiplexing action continues till the drug delivery is over. Post which all the switches will be closed (switched Off). The frequency of multiplexing will decide the flow rate with which the drug will be released. The multiplexing action will electrostatically induce a peristaltic wave along the longitudinal axis of the pump element to displace fluid disposed within the pump body. The functioning of the Electrostrictive micro pump has been already covered in detail in the other sections. A person of ordinary skill in the art will appreciate that many variations and alterations to the components and the circuit are possible.

Referring to FIG. 13D, it shows the embodiment of a simplistic representation of the control circuit for Micro Pump for microfluidic channel. The functioning of the pump has been already described in detail in the other sections. The battery 1330 provides power to the processor 1332. Based on the processor program, the control circuit 1334 is made to control the shape/displacement of the channel wall (inside the assembly 1336). The processor communicates with the controller using the communication channel (ports) 1338. The controller 1334 contains the waveform generator. The controller circuit 1334 generates and applies the actuation waveforms to change the shape of the channel wall depicted as 1336 in the diagram. The channel wall (diaphragm) displacements are proportional to the voltages applied by the controller and hence the movement/displacement will be in accordance to the type and frequency of the waveform applied. A person of ordinary skill in the art will appreciate that many variations and alterations to the components and the circuit are possible.

Referring to FIG. 13E, it shows the embodiment of a simplistic representation of the control circuit for Micro Diaphragm Pump. The functioning of the pump has been already described in detail in the other sections. The battery 1320 provides power to the processor 1322. Processor 1322, using its output port line 1328 switches on the MOSFET 1324 to actuate the actuator 1326. The switching on of the MOSFET connects the battery power to the actuator 1326. The upward movement of the actuator opens the inlet(s) inside the pump. This allows the infusion liquid to flow through the pump. Switching off the MOSFET (1324) removes the power from the actuator (1326). This makes the actuator return to its default close position, which in turn closes the inlets. This stops the liquid flow. The program residing in the processor decides the duration to which the actuator should be kept in an actuated position based on the dosage quantity.

In an embodiment, one end of the electronic unit comprises an insulation plug. In another embodiment, the insulation cap may comprise a sensor that is configured to detect a successful delivery of drug into the body of a subject. In another embodiment, the data obtained using the sensor can be used to generate a closed loop for drug delivery.

In an embodiment, the microcontroller may be programmed using a wired or wireless connection.

Consistent amount of drug delivery per shot is an important aspect of drug administration that ensures the efficacy and safety of the medication. When a medication is prescribed, the dosage is carefully calculated based on a variety of factors such as the patient's age, weight, medical history, and the severity of the condition being treated. In order to ensure that the patient receives the correct dose of the medication, it is important to deliver a consistent amount of drug with each shot.

In an embodiment, the implantable device is designed to accurately measure and deliver the prescribed dose of medication per shot. In some embodiments, the device is equipped with safety features that help prevent over or under-dosing of the medication per shot.

In an embodiment, the dose of medication per shot is a lowest prescribed dose and can be adjusted over time up to one of a maximum prescribed dose or maintenance prescribed dose. In some embodiments, a caregiver or a patient can make the device change prescribed dose of medication per shot during the life of the implantable device while the device is implanted.

In an embodiment the wireless communication unit within the implanted device may have a printed antenna printed on the outer surface of the implant body.

### K. Device Comprising Piston Position Determination Module

In an embodiment, the pressure transducer could be mounted on the inside wall of the osmotic unit and the cavities as donut shaped discs.

Another embodiment relates to a device comprising a first chamber comprising a drug reservoir unit and an osmotic unit; a second chamber comprising an electronic unit; a third chamber comprising a drug delivery unit; and a piston position determination module; and wherein the device is configured to deliver a drug into a body of a subject; wherein the device is implantable into the body of the subject; and wherein the device is configured to mimic a flow pattern of repeated injections to deliver a constant volume of the drug during each injection of the repeated injections.

In an embodiment, the implantable device may comprise a piston position determination module. The piston position determination module can be based on one of a pressure measurement, capacitance measurement, impedance measurement, radical measurement, image-based measurement, light reflection measurement, laser measurement, SONAR based measurement, ultrasound measurement, and time of flight measurement.

Below are described various embodiments related to piston position determination module.

### 1. Ultrasound Distance Measurement:

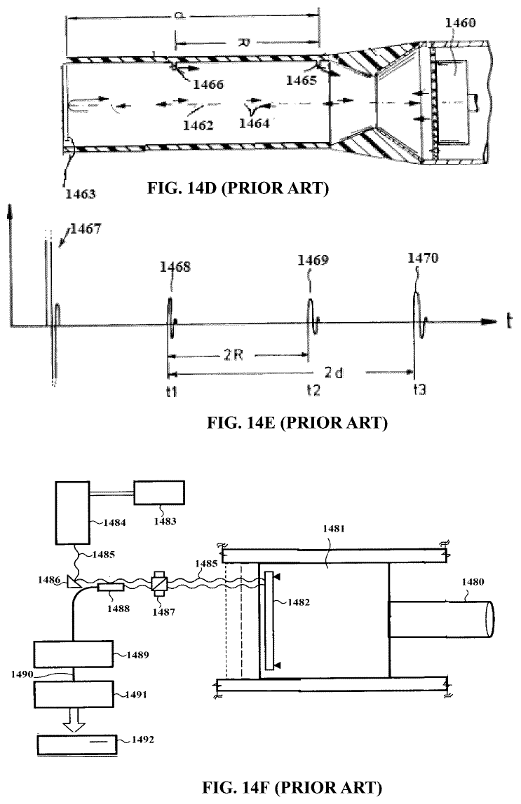

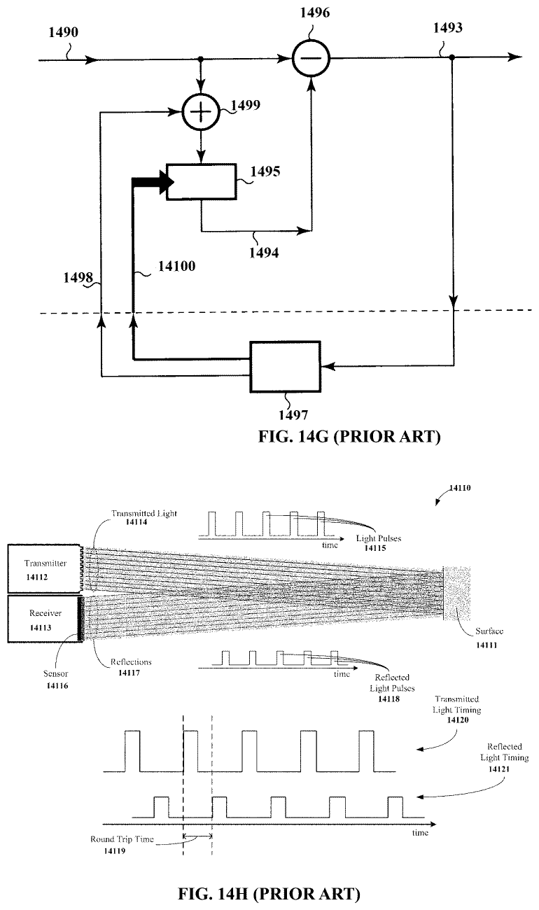

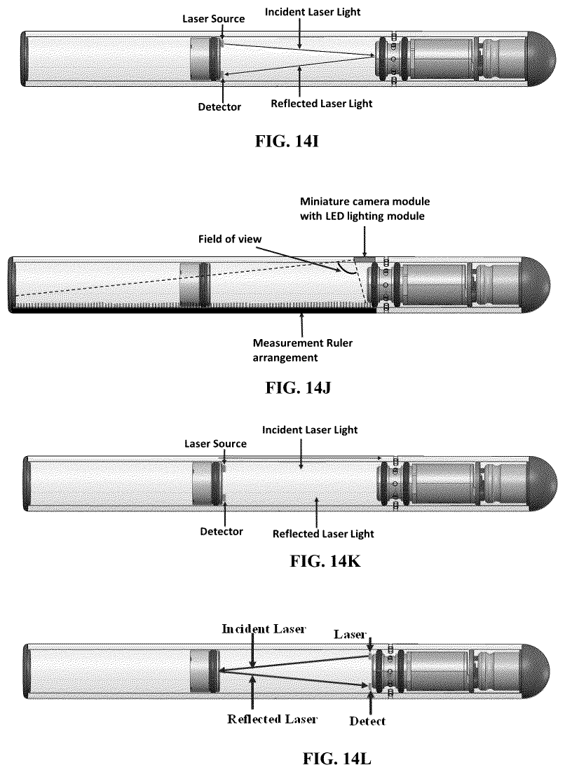

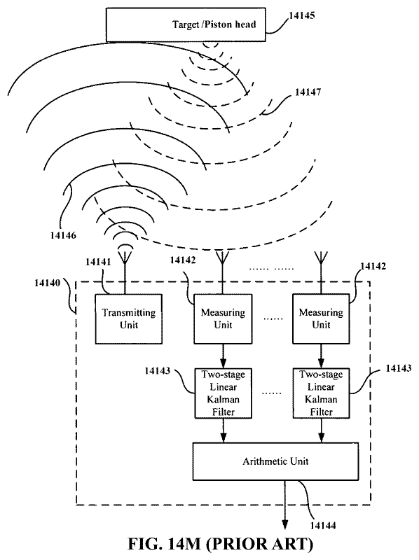

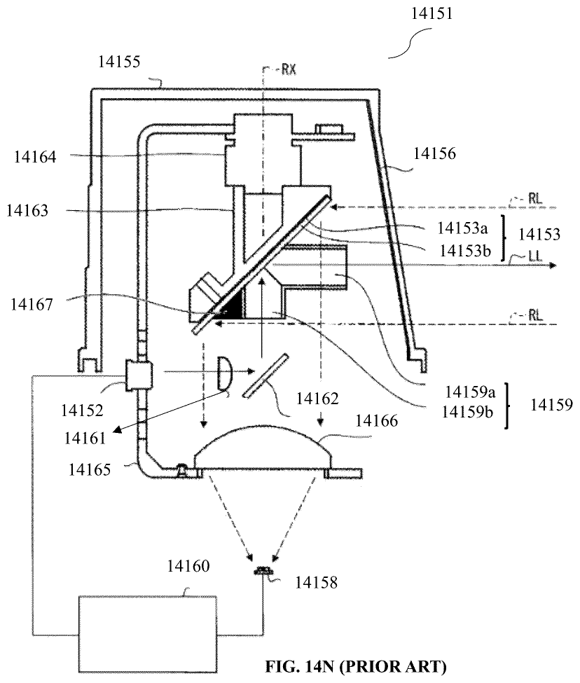

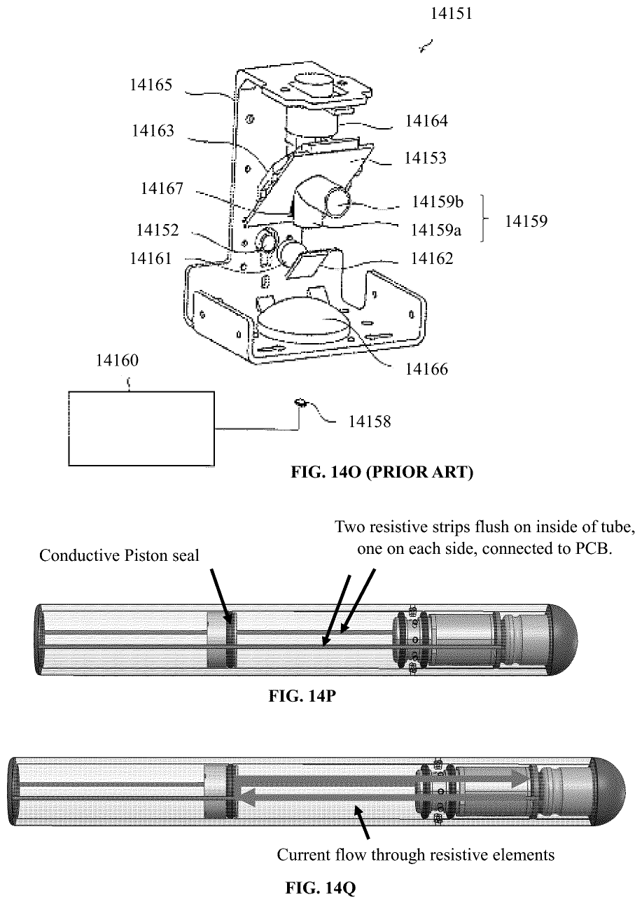

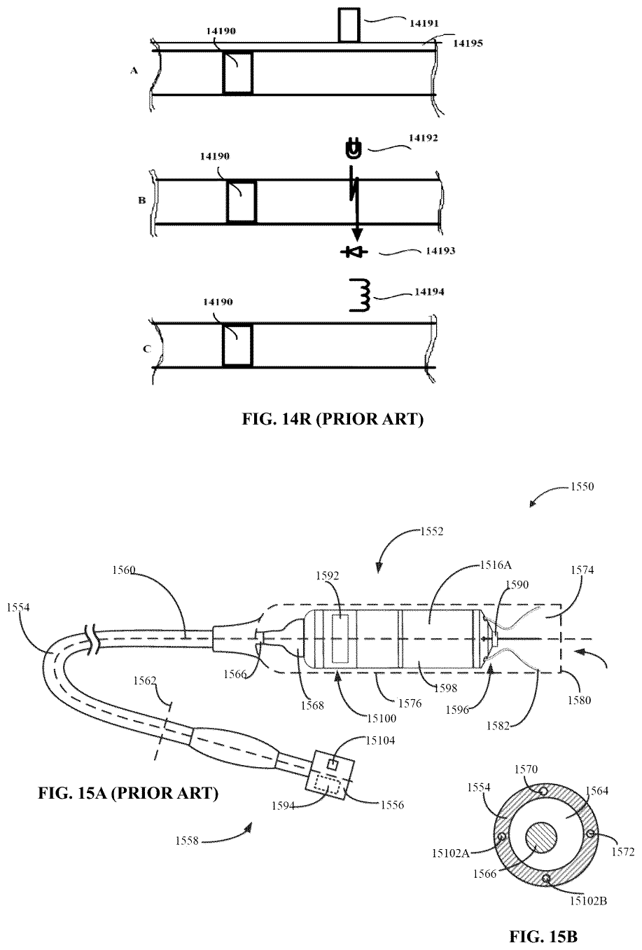

FIG. 14A is a schematic view of an embodiment of the piston position determination module, according to one or more embodiments, and FIG. 14B illustrates an alternative embodiment of the device of the figure.

In FIG. 14A, there is shown very schematically a measuring probe comprising a housing 1410 of generally parallelepipedal shape, the lower wall of which has a circular opening 1412. The probe is placed with the opening 1412 facing a surface S (of a piston) of a product P, and placed close to this surface, in order to measure the distance between this surface S and a fixed origin, for example an origin linked to the probe. The product P can be a movable piston within the implantable device, the distance measurement being used to detect movement of the piston.

A first ultrasonic translator 1414 is fixed through the upper wall of the housing 1410. The active face of translator 1414 consists of a piezoelectric disc 1416 located inside housing 1410 and facing opening 1412. The ultrasonic signal produced by patch 1416 is thus transmitted to surface S, through opening 1412, and is reflected by surface S in the direction of translator 1414 acting as transmitter and receiver.

Translator 1414 and surface S are not in direct contact to avoid abrasion or damage to the piezoelectric patch. Also, as is known per se, a coupling fluid C is interposed between the translator 1414 and the surface S to facilitate the transmission of ultrasound. In the example illustrated, the coupling fluid C is a liquid, such as water, which flows continuously between an inlet 1418 formed in the upper wall of the housing 1410 and the opening 1412. The interior volume of the case as well as the space between the opening 1412 and the surface S are thus permanently occupied by the coupling liquid. In order to avoid the formation of bubbles which would disturb the transmission, an effort is made to achieve a “quiet” flow inside the housing 1410. To this end, the coupling liquid C can be admitted into the casing 1410 after passing through a “tranquilization” chamber (not shown).

A second ultrasonic transducer 1420 is attached through a side wall 1410a of housing 1410. The active face of translator 1420 consists of a piezoelectric disc 1422 located inside housing 1410. The pellet or disc 1422 faces a reference surface S′ formed on the inside of the side wall 1410b of the housing 1410 opposite the wall 1410a, the coupling between the pellet 1422 and the surface S′ being achieved by the coupling liquid C filling box 1410. The surface S′ is, for example, the rectified surface of a projecting part or internal boss of the wall 1410b. In this way, a signal emitted by the translator 1420 is received by the latter after reflection on the surface S′, before any other echo coming from a reflection on another part of the inner face of the wall 1410b.

The translators 1414 and 1420 are connected to a measurement circuit 1424 via an inverter 1426. In one position, the inverter 1426 connects the measurement circuit 1424 to the translator 1414 to control the emission of an ultrasonic pulse and to determine a quantity representing the propagation time T of this pulse between the translator 1414 and the surface S. In the same way, in its other position, the inverter 1426 connects the measuring circuit 1424 to the transducer 1420 to control the emission by the latter of an ultrasonic pulse and to determine a quantity representing the propagation time T ‘of this pulse between the translator 1420 and the reference surface S’.

The measurement circuit 1424 is of an entirely conventional type, so that a detailed description is not necessary here. It comprises a processing and control circuit 1430, for example with a microprocessor, which controls a generator circuit 1426 to apply an electrical voltage pulse to the piezoelectric disc of the transducer 1414 (or 1420) in order to cause the emission of an ultrasonic pulse. The electrical signals produced by the patch in response to the reception of these echoes of each transmitted pulse are received by the circuit for this 3D processing to determine the propagation time (or T′).

The measured propagation time T is, for example separating two successive ultrasonic echoes (or not). The distance Y between the transducer 1414 and the surface S is then Y=V T 2n, n being equal to the number of double paths of the ultrasonic pulse between the two echoes separated by the time T, and V being the speed of propagation of the ultrasounds in the coupling fluid C.

Similarly, the distance X between the transducer 1420 and the surface S′ is of the form X=V T′ 2n′, n′ being equal to the number of double paths of the ultrasonic pulse between the two echoes separated by the time T′.

It will be noted, as a variant, that the propagation times can be determined between the emission of the ultrasonic pulse and the reception of the first echo, or of a predetermined following echo.

The times T and T′ are measured successively, by switching the inverter 1426. The distance Y sought is then calculated by means of the processing device 1430. The calculated value Y is transmitted to a digital display 1432 and/or a graphic recorder 1434.

The distance X has a predetermined fixed value. In order to avoid any variation in this distance during use, the transducer 1420 is sealed to the wall 1410a of the housing, for example, by gluing, and the housing is made of a material having a high dimensional stability with respect to the temperature, for example in “Invar”.

In the example illustrated, the ultrasonic waves produced by the transducers 1414 and 1420 have different directions of propagation.

As a variant, and as illustrated by FIG. 14B, the directions of propagation of the waves produced by the two translators 1414 and 1420 can be parallel. In this case, the translator 1420 is fixed through the upper wall of the casing 1410 with its active face turned towards the lower wall and facing the reference surface S′ formed on a raised part of this lower wall, on the inner side. For the rest, the device of FIG. 14B is identical to that of FIG. 14A, the same reference signs designating the same elements in the two figures.

### 2. Distance Measuring by Image Processing:

In FIG. 14C, the distance measuring device includes a lens array 1441, an image sensor 1445, and a substrate 1448.

The lens array 1441 is formed by integrally forming two distance measuring lenses (lens 1442a, lens 1442b). The lens 1442a and the lens 1442b have the same shape and the same focal length. The optical axis 1444a of the lens 1442a and the optical axis 1444b of the lens 1442b are parallel, and the distance between them is the baseline length D.

In this specification, when indicating the direction of the distance measuring device, the axis along the optical axis 1444a and the optical axis 1444b is defined as the Z axis, and the direction perpendicular to the Z axis and from the optical axis 1444a toward the optical axis 1444b is defined as the Y axis. When the direction orthogonal to both the Z-axis and the Y-axis is the X-axis, the positive direction of the X-axis is upward and the negative direction of the X-axis is downward. The lens 1442a and the lens 1442b are arranged on the XY plane, and the centers of both lenses are arranged on the Y axis. Therefore, a state where the lens 1442a and the lens 1442b are oriented in the Z-axis direction and a state where the lens 1442a and the lens 1442b are on the Y-axis is a normal use state. The state in which the lens 1442a and the lens 1442b are oriented in the Z-axis direction and the state in which the lens 1442a and the lens 1442b are on the X-axis is a state in which the distance measuring device is used by being inclined by 90 degrees. In the normal state, the parallax Δ of the distance measuring device occurs in the Y-axis direction.

The image pickup device 1445 is an image pickup device such as a CMOS or a Charged Coupled Device (CCD) and includes a large number of light receiving elements (pixels) formed on a silicon wafer by a semiconductor process. In this embodiment, a CCD is used for the image sensor 1445. The imaging element 1445 has two light receiving surfaces 1446a and 1446b. An image of an object that has passed through the lens 1442a is formed on the light receiving surface 1446a. An image of an object that has passed through the lens 1442b is formed on the light receiving surface 1446b. The light receiving surface 1446a and the light receiving surface 1446b are arranged with a distance corresponding to the base line length D of the lens 14442a and the lens 1442b.

The light receiving surface 1446a and the light receiving surface 1446b are formed in a rectangular shape having the same size, and the diagonal centers of the light receiving surface 1446a and the light receiving surface 1446b are arranged so that the optical axes of the lenses 1442a and 1442b substantially coincide with each other. Thus, by arranging the light receiving surface 1446a and the light receiving surface 1446b apart from each other, it is not necessary to provide a wall or the like that prevents the incident light beam from leaking to the adjacent light receiving surface.

The substrate 1448 includes a digital signal processor (DSP) that performs image processing on the image signal output from the image sensor 1445. The DSP provided on the substrate 1448 is configured to perform distance measurement by performing processing on the image signal based on the subject image formed on the light receiving surfaces 1446a and 1446b via the lens array 1441.

### 3. Distance Measurement in Liquid (Ultrasonics):

Referring to FIG. 14D, that shows ultrasonic distance measurement, a sensor 1460 transmitting and receiving in the exemplary embodiment shown is subjected to an electrical transmission pulse, which is converted into an acoustic sound pulse. This propagates along a direction of propagation 1462, downwards in FIG. 14D, and reaches an object 1463. There, a part of the sound pulse is reflected in the direction of propagation 1462 and again arrives in sensor 1460. The propagation of the sound is indicated by arrows 1464.

In the direction of propagation 1462 there are a first and a second auxiliary reflector 1465, and 1466 in front of the sensor 1460. They are the same and in one fixed distance R mechanically arranged permanently and precisely from each other. As shown in FIG. 14D, both auxiliary reflectors 1465, 1466 are constructed identically; the distance R is measured from a smooth surface that runs transversely to the direction of propagation 1462 and faces the sensor 1460. Also shown in FIG. 14D is the distance d between the described front surface of the first auxiliary reflector 1465 and the surface of the object 1463. Distance d is the measurement variable.

Part of the ultrasound pulse is reflected on the two auxiliary reflectors 1465 and 1466; this is indicated by corresponding arrows. The reflected portion that reaches sensor 1460 is converted into an electrical signal. The individual signals are explained with reference to FIG. 14E wherein 1467 denotes the actual transmission signal, which, however, does not play a role according to the invention; rather, the invention enables any reference to this transmission signal to be slipped out. In terms of the device, this in turn gives the possibility of completely blocking the reception channel during the time period of the transmission signal and of opening it only after a dead time that is somewhat shorter than the time t¹ still to be discussed.

As a first echo after the transmission of a sound pulse, the sound component reflected at the first auxiliary reflector 1465 reaches the sensor 1460, it leads to an electrical signal 1468 which occurs at time t¹. The sound component reflected by the second auxiliary reflector 1466 arrives next in the sensor 1460, where it leads to an electrical signal 1469 which occurs at time t². After that, the echo from object 1463 arrives, which leads to an electrical signal 1470.

The time period t² minus t¹ is the double path R, that is to say the reference distance, and accordingly this time period is also designated 2R in FIG. 14E. Since the distance R between the two auxiliary reflectors 1465, 1466 is specified as precisely as possible, that is to say mechanically and thermally stable, the speed of sound v can be calculated over the time period t² minus t¹ and the distance 2R. The speed of sound obtained in this way is multiplied by the time period t³ minus t¹ and results in the distance of the object 1463 from the first auxiliary reflector 1465. The measurement is thus completed.

According to the invention, the distance R is thus measured with one and the same sound pulse and the respective speed of sound is calculated from this, and the time period with which the sound travels the distance 2d is determined. Measurement and reference measurement are therefore carried out simultaneously, so that any temperature differences in the running distance are averaged out in the actual measurement result without inertia. Furthermore, only acoustic echoes (and not the transmission pulse) are used for the measurements.

The reference path R is chosen to be as large as possible, the larger it is chosen, the more precise the temperature compensation, and the more precisely the measuring path d is detected. The section 2d-2R should be as small as possible, the section 2R as large as possible.

The design of the auxiliary reflectors 1465, 1466 is itself arbitrary. It has proven to be very advantageous to arrange the auxiliary reflectors in a tube section of an outlet part. The auxiliary reflectors can be locally arranged webs or can run in a ring on the inner wall of the tube, they can be designed as grids, as spokes that extend into the vicinity of the center of the tube or the like. Basically, the geometrical design of the auxiliary reflectors 1465, 1466 is arbitrary, with their construction's only care that must be taken is that they are mechanically and thermally stable.

From the above it follows that the location at which the sensor 1460 is located is of no importance for the measurement. Only the constancy of the distance R of the auxiliary reflectors 1465, 1466 is decisive for the measuring accuracy, because this distance multiplied by the ratio of the running times t³ minus t¹ to t² minus t¹ gives the measuring distance d. A special adjustment of the sensor 1460 within the housing of the measuring head is therefore not necessary.

### 4. Distance Measurement Using Laser Interferometer:

The overall exemplary configuration of a laser interferometer displacement measuring system according to the present invention is shown in FIG. 14F. In the figure shown is an actual example in which laser displacement measurement is employed for measuring a piston head position with accuracy. In an embodiment, the device may be performing feedback control. The piston head is subjected to a driving power source 1480 and detects the distance of a movable piston head 1481 using variations in position of a reflector 1482 on the stage table.

A laser power supply 1483 drives a gas laser light source 1484 to generate laser light 1485, which is in turn reflected on a beam bender 1486 and then introduced into an interferometer 1487. The optical path is divided into two paths inside the interferometer 1487. One of the optical paths is the measurement path in which the light reaches the reflector 1482 on the piston head 1481 and is then reflected thereon to return to the interferometer 1487. The other optical path is the reference path in which the light is reflected inside the interferometer 1487. This example employs a four-fold optical path (in which light travels twice between the interferometer 1487 and the reflector 1482) for laser displacement measurement. The light beams of the measurement path and the reference path are mixed in the interferometer 1487. Mixing the two light beams causes interference to occur. The light having caused the interference is launched from the interferometer 1487 and then detected by a light detector 1488. The light detector 1488 detects the light and converts the detected amount of light into an electrical signal. A measuring board 1489 converts the resulting electrical signal into a coordinate value and then outputs the resulting value as a measurement value 1490. Means for increasing displacement output value accuracy 1491 corrects the measurement value 1490 to output a measurement value with increased accuracy. A personal computer, for collecting data and performing control, captures the value to perform feedback to the position of the stage and a correction mechanism. Incidentally, the measuring board 1489 is generally called a counter board or an axis board but is herein consistently referred to as the measuring board.

The present invention relates particularly to a correction method and means employed in the means 1491 for increasing displacement output value accuracy. In this embodiment, this is hereinafter described as independent processing means. This is because this function is implemented in the form of electrical signal processing and can be implemented by either hardware or software. Thus, it is made possible to implement this function by incorporating the means 1491 as hardware into the measuring board 1489 or by incorporating the means 1491 as software into the processor 1492. It is also possible to use independent hardware to implement an independent configuration.

The laser light source 1484 employed in this embodiment is a He—Ne gas laser for emitting laser light at a wavelength of 633 nm. A high-frequency electromagnetic wave is applied to the gas for excitation. Incidentally, a vacuum chamber can be employed to seal the interferometer 1487 and the subsequent portions (the interferometer 1487, the piston head 1481, and the reflector 1482) therein to provide the measurement path in a vacuum, thereby making it possible to prevent an error in measurement caused by variations in refractivity due to a fluctuation of air or a change in humidity of air. Furthermore, to maintain the accuracy of measurement, a multi-layered coating is applied to the components such as the beam bender 1486, the interferometer 1487, and a transparent window attached to the wall of the vacuum chamber in order to prevent unnecessary multiple reflections.

The laser interferometer displacement measuring system configured as such makes it possible to provide measurement values or coordinate outputs at a resolution of 0.3 nm and a high sampling rate of 10 MHz.

A method for processing a signal to implement the correction is shown in FIG. 14G. FIG. 14G shows an example of the means 1491 for increasing displacement output value accuracy, by which the measurement value 1490 output from the measuring board 1489 is captured to output a measurement value 1493 with increased accuracy. In accordance with the captured measurement value 1490, a correcting value 1494 is read from a memory device 1495 and corrected by an adder/subtractor 1496, then being output as the measurement value 1493 with increased accuracy. Designated as 1497 is a parabolic component extracting filter unit for outputting a phase shifting value 1498.

The correcting value 1494 is generated as described below. First, a phase adder 1499 adds the phase shifting value 1498 to the measurement value 1490 and then outputs the resulting value as a table reference address. In accordance with the table reference address, the memory device 1495 outputs the correcting value 1494 stored therein. The memory device 1495 has data stored therein, which is to be outputted as the correcting value 1494 and which provides a cyclic value corresponding to the wavelength cycle of laser light. The value can be set to a given one presetting value 14100. It is desirable that the table reference address be cyclic in accordance with the cycle of the laser wavelength. In this regard, only the upper bits equal to or greater than the wavelength cycle can be ignored when the measurement value of laser interferometry employs those output digitally with two to the power of N being adopted as the wavelength cycle. More specific values to be stored in the table are the waveforms (or one cycle of the waveform), which may be stored as they are. Besides this, a sinusoidal (sine) waveform having the same cycle can be employed to allow the correction to provide the same effect of increasing accuracy.

### 5. Distance Measurement Using Time of Flight (TOF):

One alternative to a structured light system is a TOF system. FIG. 14H is a depiction of an example TOF system 14110. The TOF system 14110 may be used to generate a depth map (not pictured) of a scene (with surface 14111 in the scene) or may be used for other applications for ranging surface 14111 or other portions of the scene. The TOF system 14110 may include a transmitter 14112 and a receiver 14113. The transmitter 14112 may be referred to as a “transmitter,” “projector,” “emitter,” and so on, and should not be limited to a specific transmission component. Similarly, the receiver 14113 may be referred to as a “detector,” “sensor,” “sensing element,” “photodetector,” and so on, and should not be limited to a specific receiving component.

The transmitter 14112 may be configured to transmit, emit, or project signals (such as a field of light) onto the scene (including surface 14111). While TOF systems are described in the examples as emitting light (which may include near-infrared (NIR)), signals at other frequencies may be used, such as microwaves, radio frequency signals, sound, and so on. The present disclosure should not be limited to a specific range of frequencies for the emitted signals.

The transmitter 14112 transmits light 14114 toward a scene. While the transmitted light is illustrated as being directed to surface 14111, the field of the emission or transmission by the transmitter extends beyond as depicted for the transmitted light 14114. For example, conventional TOF system transmitters have a fixed focal length lens for the emission that defines the field of the transmission from the transmitter. The fixed field of the transmission for a conventional TOF system is larger at a depth from the transmitter than the fixed field of transmission for each point of the spatial distribution for a conventional structured light system. As a result, conventional structured light systems may have longer effective ranges than conventional TOF systems.

The transmitted light 14114 includes light pulses 14115 at known time intervals (such as periodically). The receiver 14113 includes a sensor 14116 to sense the reflections 14117 of the transmitted light 14114. The reflections 14117 include the reflected light pulses 14118, and the TOF system determines a round-trip time 14119 for the light by comparing the timing 14120 of the transmitted light pulses to the timing 14121 of the reflected light pulses. The distance of the surface 14111 from the TOF system may be calculated to be half the round-trip time multiplied by the speed of the emissions (such as the speed of light for light emissions).

The sensor 14116 may include an array of photodiodes to measure or sense the reflections. Alternatively, the sensor 14116 may include a CMOS sensor or other suitable photo-sensitive sensor including a number of pixels or regions for sensing. The TOF system 14110 identifies the reflected light pulses 14118 as sensed by the sensor 14116 when the magnitudes of the pulses are greater than a threshold. For example, the TOF system measures a magnitude of the ambient light and other interference without the signal, and then determines if further measurements are greater than the previous measurement by a threshold. However, the noise or the degradation of the signal before sensing may cause the signal-to-noise ratio (SNR) to be too great for the sensor to accurately sense the reflected light pulses 14118.

To reduce interference, the receiver 14113 may include a bandpass filter before the sensor 14116 to filter some of the incoming light at different wavelengths than the transmitted light 14114. There is still noise sensed by the sensor, though, and the SNR increases as the signal strength of the reflections 14117 decreases (such as the surface 14111 moving further from the TOF system 14110, or the reflectivity of the surface 14111 decreasing). The TOF system 14110 may also increase the power for the transmitter 14112 to increase the intensity of the transmitted light 14114. However, many devices have power constraints (such as smartphones, tablets, or other battery devices), and are limited in increasing the intensity of the emitted light in a fixed field for a TOF system.

In some aspects of the present disclosure, the TOF system is configured to adjust the field of transmission or emission. In decreasing the field of transmission/focusing the light transmissions, the TOF system may extend the effective distance for ranging. In some example implementations, the TOF system may be configured to transmit light with different fields of transmissions, where a first field of transmission at a depth from the transmitter is larger than a second field of transmission at a depth from the transmitter.

Referring to FIG. 14I, a Time of Flight (ToF) device is utilized to precisely measure the distance between the piston and the valve. ToF sensors use a tiny laser to fire out light of a given wavelength where the light produced will bounce off any object and return to the sensor. Based on the time difference between the emission of the light and its return to the sensor that is located at the piston, after being reflected by a surface of the valve, the sensor is able to measure the distance between the valve and the sensor. There are 2 ways that ToF uses travel-time to determine distance and depth which are, (a) using Timed Pulses and (b) using Phase Shift of an Amplitude Modulated Wave.

Timed pulse technique works by illuminating the target with laser light and measuring the reflected light with a scanner where the distance of the object is deduced using the speed of light (c) to calculate the distance (d) traveled accurately. As can be seen in FIG. 14I, the laser light is first emitted by a laser placed at or near the piston and bounced off from the surface of the valve back to the sensor. With the laser return time, ToF device can measure the exact distance in a short time given the speed at which light travels. A timer will start during the exit of the light and when the return light is received by the receiver, the time is returned by the timer. When the two times are subtracted, the “time of flight” (t) of the light is obtained, and the speed of light is constant, so the distance is calculated using the formula (c×t)/2. With this, all points on the object's surface can be determined.

ToF can also use continuous waves to detect the phase shift of the reflected light to determine depth and distance. By modulating the amplitude, it creates a light source of a sinusoidal form with a known frequency which allows the detector/sensor to be able to determine the phase shift of the reflected light by using the formula: c=λ·f, where c is the speed of light (c=3×10 8 m/s), κ is wavelength and f is the frequency, each point on the scene can be easily calculated by the sensor to find out the depth.

In another embodiment, the same time of flight technique is used to measure the distance as described in the previous embodiment, but instead of the laser and the detector located on the piston, they are located on the valve. This arrangement has certain advantages as the PCB assembly containing the processing electronics is located close to the valve. As can be seen in FIG. 14L, the laser light is first emitted by a laser placed at or near the valve assembly and bounced off from the surface of the piston back to the sensor. With the laser return time, ToF device can measure the exact distance in a short time given the speed at which light travels. A timer will start during the exit of the light and when the return light is received by the receiver, the time is returned by the timer. When the two times are subtracted, the “time of flight” (t) of the light is obtained, and the speed of light is constant, so the distance is calculated using the formula (c×t)/2. With this, all points on the object's surface can be determined.

Referring to FIG. 14J, an integrated miniature camera and LED lighting module in conjunction with a measurement ruler arrangement with precise measurement marks on the inner surface of the drug chamber may be used to precisely measure the distance between the piston and the valve to calculate the volume of drug dispersed. The LED-lighting module will be strategically mounted at a location as shown in FIG. 14J, such that it gets the full field of view without any obstruction. Fine measurement lines will be provided on the inner circumference of the drug delivery compartment. Just before the measurement, the LED will be powered up. The camera will take a picture of the piston position and the ruler marking. This picture will be magnified approximately 500× to provide precise position. This image will be used by an image processing algorithm using the microprocessor on the PCB. The value of the exact position will be subtracted from the previous position measurement. The difference in position measurement will be used to calculate the amount of drug that has been delivered.

Referring to FIG. 14K, a modified Time of Flight (ToF) device is utilized to precisely measure the distance between the piston and the valve. In this modification, a waveguide, similar to an optical fiber, is used to couple the light directly from the laser. The light then travels through the waveguide in a controlled manner. The waveguide travels around the circumstance of the valve and returns to the detector. By measuring the total time taken by the light from generation to reaching the detector, the precise distance between the piston and the valve can be measured as summarized earlier. The advantage of this approach is that the light always travels in a controlled environment unlike the previous arrangement where it must travel through the drug compartment that could be prone to scattering and other losses.

In another embodiment a sensor for performing the distance measurement in accordance with the ultrasound echo principle, in particular for determining the distance between the piston and valve in close range with an ultrasound transmitter and receiving converter for emitting the ultrasound signals and for receiving the ultrasound signals reflected by the valve surface, whereby the converter consists of an insulated-type transformer with piezo-ceramic resonator disposed thereon.

In an embodiment, the implantable device comprises a pressure sensor that measures a pressure inside the osmotic unit and/or the drug reservoir unit. The pressure indicates a flow characteristic through the valve. This will allow the microcontroller to calculate the time period for which the valve is to be kept open for consistent drug delivery of a specific dose.

In an embodiment, the piston acts as a capacitor plate. The capacitance changes as the piston moves. The microprocessor calculates the right position of the piston based on the change in capacitance.

6. Distance Measurement using Sound Navigation and Ranging (SONAR)

SONAR stands for Sound Navigation and Ranging, which is a technology used to measure distances using sound waves. It is similar to RADAR (Radio Detection and Ranging), which uses radio waves to measure distances. SONAR works by sending out a sound wave, typically at a frequency above the range of human hearing, and then detecting the echo that is reflected back off an object. The time it takes for the sound wave to travel to the object and back is used to calculate the distance. In SONAR systems, a transmitter sends out a sound pulse, and a receiver detects the echo of the pulse after it bounces off an object. By measuring the time, it takes for the echo to return, the distance to the object can be calculated using the speed of sound in the medium through which the sound wave is traveling (such as water, air, or solid materials).

Referring to FIG. 14M, in which a block diagram of an embodiment of the distance measuring device of the present invention is shown. As shown in FIG. 14M, the device 14140 comprises a transmitting unit 14141, a plurality of measuring units 14142, a plurality of two-stage linear Kalman filters 14143 and an arithmetic unit 14144. Each of the plural two-stage linear Kalman filters 14143 supports one of the plural measuring units 14142, while the arithmetic unit 14144 is connected to the plural two-stage linear Kalman filters 14143. When the distance measuring device 14140 is used to detect a target 14145, the transmitting unit 14141 transmits a detecting pulse 14146 for detecting the target 14145 which then reflects the detecting pulse 14146 to generate a reflected pulse 14147. The above detecting pulse 14146 is frequency modulation continuous wave (FMCW). Subsequently, the plural detection units 14142 receive the reflected pulse 14147 respectively at different positions. The plural measuring units 14142 proceed with a linear frequency modulation (LFM) and a frequency-shift keying (FSK) so as to generate respectively a measured value of distance and a measured value of velocity based on the reflected pulses 14147 received. The plural two-stage linear Kalman filters 14143 proceed operation based on the measured values of distance and the measured values of velocity generated by the measuring units 14142 respectively so as to generate a plurality of distance estimation values, a plurality of velocity estimation values and a plurality of acceleration estimation values. The arithmetic unit 14144 proceeds a triangulation operation based on the plural distance estimation values, the plural velocity estimation values and the plural acceleration estimation values generated from the plural two-stage linear Kalman filters 14143 so as to generate values of distance, values of velocity and values of acceleration. In an embodiment, triangulation operation is performed by the device. Triangulation is a method used to determine the position of a point in space by measuring the angles between it and other known points.

In some embodiments, distance measurements may be taken using distance measurement devices such as LIDAR, camera, laser, sonar, ultrasonic, stereo vision, structured light vision devices or chip-based depth sensors using CMOS or CCD imagers, IR sensors, and such.

7. Distance Measurement using Light Reflection:

As shown in FIG. 14N and FIG. 14O, the distance measuring device 14151 includes a light emitting element 14152 (light emitting unit) that emits a laser beam LL. The light emitting element 14152 is attached to the side surface of the fixing member 14165 made of sheet metal. The laser light LL emitted from the light emitting element 14152 is converted into parallel light by the lens 14161. The laser beam LL converted into parallel light by the lens 14161 is reflected by the mirror 14162.

The distance measuring device 14151 includes a substantially square plate-shaped deflecting member 14153 (reflecting member) that further reflects the laser beam LL reflected by the mirror 14162. The deflecting member 14153 is disposed along a direction inclined about 45 degrees with respect to the traveling direction of the laser light LL reflected by the mirror 14162, and is attached to the attachment member 14163.

The deflection member 14153 is formed from an optical element such as a glass mirror. The deflection member 14153 includes a reflecting surface 14153a and a transparent layer 14153b formed on the surface of the reflecting surface 14153a. The reflecting surface 14153a is formed from, for example, a mirror-finished metal film such as glass, gold, or aluminum. The transparent layer 14153b protects the reflective surface 14153a, and is formed of a dielectric multilayer film in this embodiment. That is, the deflecting member 14153 of the present embodiment is a dielectric multilayer mirror in which the surface of the reflecting surface 14153a is coated with a transparent layer 14153b made of a dielectric difference layer film. Since the dielectric multilayer film has a high reflectance, the light incident on the deflecting member 14153 can be reflected with high efficiency by using the dielectric multilayer film as the transparent layer 14153b.

The attachment member 14163 is fixed to a motor 14164 that rotates around the rotation axis RX. The motor 14164 is fixed to the upper part of the fixing member 14165. The deflection member 14153 can be rotated by the motor 14164 along the rotation axis RX along the incident direction of the laser beam LL.

A laser beam passage member 14159 (optical path member) is provided at the center of the deflection member 14153. The laser beam passage member 14159 is composed of an inverted L-shaped pipe. The laser light passage member 14159 is formed with optical paths 14159a and 14159b having a circular cross section. The optical paths 14159a and 14159b are holes (tubular holes) through which the laser light LL passes. The optical path 14159a causes the laser beam LL emitted from the light emitting element 14152 to enter the deflection member 14153. The optical path 14159b emits the laser beam LL incident on the deflecting member 14153 through the optical path 14159a to the outside. The optical path 14159a allows the laser light LL to pass in the vertical direction so as to block the spreading component of the laser light LL reflected by the mirror 14162. The optical path 14159b passes the laser beam LL in the horizontal direction so as to block the spread component of the laser beam LL reflected by the deflecting member 14153.

As described above, when the laser beam LL reflected by the mirror 14162 enters the laser beam passage member 14159, the laser beam LL passes through the optical path 14159a and is guided to the deflection member 14153. The laser beam LL reflected by the deflecting member 14153 passes through the optical path 14159b and is emitted to the outside.

The laser light passing member 14159 has an opening formed by obliquely cutting a bent portion of the inverted L-shaped pipe (joint portion between the optical path 14159a and the optical path 14159b). The laser light is in contact with the center of the surface of the deflection member 14153. Between the surface of the deflection member 14153 and the outer surface of the optical path 14159a, a light shielding portion 14167 that shields stray light generated by the deflection member 14153 is formed. As will be described later, in an embodiment, the light shielding portion 14167 is coated with an adhesive. Therefore, the deflection member 14153 and the laser beam passage member 14159 (optical path 14159a) are bonded in the region where the light shielding portion 14167 is formed. Details of the light shielding unit 14167 will be described later.

The distance measuring device 14151 is provided with a cover 14155 provided so as to cover the light emitting element 14152, the deflecting member 14153, the laser light passing member 14159, the mounting member 14163, the motor 14164, and the fixing member 14165. In FIG. 14O, the cover 14155 is omitted. The cover is formed with a light transmitting portion 14156 (in FIG. 14N) for transmitting the laser light LL that has passed through the laser light transmitting member 14159.

The laser beam LL that has passed through the optical path 14159b of the laser beam passage member 14159 is transmitted through the light transmitting portion 14156 of the cover 14155 and projected to the external space. If there is an object in the external space, the laser beam LL is reflected by the surface of the object, and the measurement beam RL that is the reflected beam is transmitted again through the light transmitting portion 14156 of the cover 14155 from the opposite direction to the laser beam LL.

As described above, the laser light LL emitted from the light emitting element 14152 and the measurement light RL reflected by the object in the external space travel on the same path, and both pass through the light transmitting portion 14156 of the cover 141515. The transmitted measurement light RL is reflected by the deflecting member 14153, and the reflected light is collected by the lens 14166 fixed to the bottom of the fixing member 14165 and reaches the light receiving element (light receiving unit) 14158.

The distance measuring device 14151 has an arithmetic processing device 14160. Arithmetic processing device 14160 is based on the time difference between the laser pulse of laser beam LL projected from light emitting element 14152 and the laser pulse of measurement light RL reflected by the object and received by light receiving element 14158.

### 8. Distance Measurement Using Resistance

According to an embodiment, two separate resistive traces connected to the PCB run longitudinally along the inside of the tube. The piston seal (or other conductive elements such as brushes) electrically connects the two traces. As the piston moves longitudinally along the tube, the resistance seen by the PCB across these two traces changes. This resistance is correlated to piston position within the tube. Piston position is used to determine pharmaceutical remaining in the pharmaceutical reservoir. Other configurations using resistance may also provide position data.

In an embodiment, the piston position determination module is based on resistance measurement.

Referring to FIG. 14P, the device comprises a plurality of resistive bands leaving from the PCB towards the first end such that the piston seal slides along the resistive bands, still a smooth non frictional edge. The resistive bands may be slightly inserted into the internal walls of the implantable device. These resistive elements are stationary. Conductive elements can be attached to a seal of the piston according to some embodiments. In some embodiments, the piston comprises a conductive element that connects those two resistant bands. As the piston moves closer to the valve (i.e., drug amount is decreasing), the resistance measured by the printed circuit board also decreases.

Referring to FIG. 14Q, the current flows through the resistive bands that are stuck to the sides on the internal walls of the implantable device. As the piston moves towards the valve, the current flows through the resistive band. With continued movement of the piston towards the valve the resistive band size, that the current has to go through, gets shorter. Therefore, as the piston moves, the resistance changes. The microprocessor calculates the right position of the piston based on the change in resistance.

In an embodiment, capacitance, impedance, and a pressure may be used for indicating piston position.

Pressure Based Position Sensing: Knowing pressure (from a sensor) and the flow vs pressure characteristics of the valve provides the processor with the needed information to determine the correct valve pulse timing. Also, the pressure of either fluid may be sensed although the osmotic fluid pressure avoids issues that might occur with variable piston friction.

Capacitance Based Position Sensing: According to an embodiment, a cylindrical plate placed on the piston's cylindrical sides (and connected through a trace back to the PCB), and a tapered cylindrical plate on the tube forms a capacitor that changes value as the piston moves within the tube. This capacitance is measured by the PCB and correlated to piston position.

Other methods: Many other methods exist for position sensing and can be calibrated for size and function well within the body. Technologies include laser range finding, impedance measurement, light reflection angle, sonar, etc.

### 9. Distance Measurement Using Other Means

In FIG. 14R, A-C are shown, in accordance with some embodiments of the present invention, sensors for detecting the movement of an object 14190 of an exemplary fluid flow meter. FIG. 14R, A shows a general schematic view of an object 14190, a piston head, and a sensor 14191. FIG. 14R, B shows an example of the sensor 14191 shown in FIG. 14R, A, wherein the sensor comprises a light source such as a LED 14192 and a light sensor such as a photodiode 14193. When the obstructing object moves in between the LED 14192 and the photodiode 14193, the light emitted from the LED 14192 onto the photodiode 14193 will be obstructed by the obstructing object 14190 and therefore there may be degradation in the amount of light detected by the photodiode indicating that the obstructing object 14190 position. FIG. 14R, C shows an example of the sensor 14191 shown in FIG. 14R, A, wherein the sensor comprises a coil 14194 and the object 14190 is made of metal, a magnet, or a ferromagnetic material. When the object 14190 moves substantially close to the coil 14194, a change in the magnetic field may be detected by the coil 14194 indicating the object 14190 position.

The sensor may be arranged moving along the axis of the piston movement as shown in 14195 according to an embodiment and the piston head may be the receiver to indicate the position. In another embodiment, the sensor may be in the piston and the receiver may be a strip along the body of the tube. In another embodiment, the sensor may be a continuous strip arranged all along the axis of the piston movement along the body of the tube. In another embodiment, the sensor may be moved to a location where the piston must reach for a single dose. In the most basic configuration, the position of the piston is calculated from knowledge of the expected pressure drop as the osmotic chamber increases, combined with how long the valve has been opened. In an embodiment, variation in piston friction and the concentration of solutes in the interstitial fluids entering the osmotic chamber are calculated. Feedback from a position sensor allows knowledge of both fluid remaining and fluid amount, as it is released.

In an embodiment, the implantable device comprises a pressure sensor that measures a pressure inside the osmotic unit and/or the drug reservoir unit. The pressure indicates a flow characteristic through the valve. This will allow the microcontroller to calculate the time period for which the valve is to be kept open for consistent drug delivery of a specific dose.

In an embodiment, the piston acts as a capacitor plate. The capacitance changes as the piston moves. The microprocessor calculates the right position of the piston based on the change in capacitance.

In an embodiment, position sensing of a piston in an implantable device is done using a photo image sensor.

In an embodiment, the implantable device can be implanted into an internal organ or tissue using an implant delivery device. In an example, the implant delivery device comprises an elongated member, a deployment bay that is housing the implantable device, two electrodes, and circuitry that is configured to use impedance of an electrical signal between the two electrodes to determine if one of the deployment bays or implantable devices are defining a fixation configuration.

FIGS. 15A and 15B depict an example implant delivery device 1550 for delivering implantable device 1516A. Implant delivery device may include deployment bay 1552 that is configured to house at least a portion of implantable devices and deploy implantable devices. Deployment bay 1552 may be connected to a distal portion of elongated member 1554 of implant delivery device. Deployment bay 1552 and elongated member 1554 may be configured to navigate an intravascular system of a subject. For example, a clinician may navigate deployment bay 1552 and elongated member 1554 through an intravascular system until deployment bay 1552 is at a target site in a subject in order to deploy implantable devices from deployment bay 1552. A clinician may use hub 1556 of implant delivery device to handle implant delivery device and/or navigate deployment bay 1552 to a target site. Hub 1556 of implant delivery device may be at proximal portion 1558 of implant delivery device. In some examples, hub 1556 may be configured to remain external to subject as deployment bay 1552 is navigated to the target site, enabling a clinician to navigate an intravenous system and deploy implantable devices using one or more mechanisms or ports (not depicted) of hub 1556. Deployment bay 1552 may be located at or near a distal end of implant delivery device, and hub 1556 may be located at or near a proximal end of implant delivery device, with elongated member 1554 extending between hub 1556 and deployment bay 1552.

Elongated member 1554 may be a flexible elongated component that longitudinally extends along implant delivery device. Elongated member 1554 may extend between deployment bay 1552 and hub 1556 of implant delivery device along longitudinal axis 1560 of implant delivery device. Elongated member 1554 may be substantially cylindrical such that elongated member 1554 defines a substantially circular cross-sectional shape. In other examples, elongated member 1554 may define one or more other cross-section shapes, including defining a plurality of cross-sectional shapes along a longitudinal length of elongated member 1554.

Elongated member 1554 may define a number of longitudinal lumens for a variety of purposes. For example, as depicted in conceptual cross-sectional view of FIG. 15B, as taken along cutline 1562, elongated member 1554 may define lumen 1564 that occupies a majority of a cross-sectional width of elongated member 1554. Lumen 1564 may be configured to house deployment mechanism 1566 that can axially slide (e.g., slides along longitudinal axis 1560 of implant delivery device) within lumen 1564 relative to implant delivery device. In this way, a clinician may, e.g., deploy implantable devices from deployment bay 1552 once the clinician navigates deployment bay 1552 to a target site as a result of an action executed using deployment mechanism 1566. For example, deployment mechanism 1566 may include pushing element 1568 at a distal end of deployment mechanism 1566. Pushing element 1568 may be configured to contact and impart a distal force on a proximal face of implantable devices in response to deployment mechanism 1566 being slid distally within lumen 1564. Additionally, or alternatively, deployment mechanism 1566 may include a tether or catch that is used to hold, push, retract, or otherwise move implantable devices relative to deployment bay 1552.

Further, in some examples, holes 1586A, 1586B (collectively, “holes 1586”) may improve a manner in which deployment bay 1552 is withdrawn from subject. For example, as deployment bay 1552 is withdrawn from subject (e.g., following the deployment of implantable device 1516A), distal opening 1598 may create a relatively negative pressure within deployment bay 1552 that pulls some fluid or tissue of subject into deployment bay 1552. Once deployment bay 1552 is fully withdrawn out of subject (e.g., through an introducer sheath or the like) this relatively negative pressure may stabilize, causing any fluid or tissue that has been pulled into deployment bay 1552 to be leaked or ejected from deployment bay 1552. Holes 1586 may improve an ability of pressure within deployment bay 1552 to normalize (e.g., be relatively equal within and outside of deployment bay 1552) as deployment bay 1552 is withdrawn from subject, therein reducing or eliminating a vacuum effect that may pull fluid or tissue into deployment bay 1552. Reducing or eliminating this vacuum effect may reduce or eliminate the likelihood that deployment bay 1552 gathers and then leaks and/or ejects subject fluid or tissue as a result of withdrawing deployment bay 1552 from subject.

In some examples, implant delivery device 1550 may be configured to verify when deployment bay 1552 presses into tissue of a subject with a threshold amount of force.

In some examples, an implant delivery device includes an elongated member configured to navigate an intravascular system of a patient. The delivery system also includes a deployment bay connected to a distal portion of the elongated member and configured to house at least a portion of the implantable device. The deployment bay defines a distal opening 1598 configured for deployment of the implantable device out of the deployment bay at a target site in a patient. The delivery system also includes a first electrode 1590 located inside of the deployment bay as the elongated member navigates the intravascular system. The delivery system also includes a second electrode 1592. The delivery system also includes signal generation circuitry 1594 configured to deliver an electrical signal to a path between the first electrode 1590 and the second electrode 1592 through at least one of a fluid or tissue of the patient. The delivery system also includes processing circuitry 1594 configured to determine an impedance of the path based on the signal and control a user interface 15104 to indicate when an impedance of the path indicates that at least one of the implantable devices or the distal opening 1598 is in a fixation configuration relative to the target site of the patient.

In other examples, an implant delivery device includes an elongated member configured to navigate an intravascular system of a patient. The delivery system also includes a deployment bay connected to a distal portion of the elongated member and configured to house at least a portion of an implantable device. The deployment bay defines a distal opening 1598 configured for deployment of the implantable device out of the deployment bay at a target site in a patient. The delivery system also includes a first electrode 1590 secured to an inner surface of the deployment bay. The delivery system also includes a second electrode 1592 secured to an outer surface of either the deployment bay or the elongated member. The delivery system also includes signal generation circuitry 1594 configured to deliver an electrical signal to a path between the first electrode 1590 and the second electrode 1592 through at least one of a fluid or tissue of the patient. The delivery system also includes processing circuitry 1594 configured to determine an impedance of the path based on the signal and control a user interface 15104 to indicate when an impedance of the path indicates that the distal opening 1598 is in a fixation configuration relative to the target site of the patient.

In other examples, an implant delivery device includes an implantable device configured to be secured to a target site in a patient via a plurality of fixation elements 1582 extending distally from a distal tip 1596 of a housing of the implantable device 1598. The delivery system also includes a first electrode 1590 secured to the distal tip 1596 of the housing 1598. The delivery system also includes a second electrode 1592 secured to an outer surface of a proximal portion 15100 of the housing 1598. The delivery system also includes signal generation circuitry 1594 configured to deliver an electrical signal to a path between the first electrode 1590 and the second electrode 1592 through at least one of a fluid or tissue of the patient. The delivery system also includes processing circuitry 1594 configured to determine an impedance of the path based on the signal and control a user interface 15104 to indicate when an impedance of the path indicates that the implantable device is in a fixation configuration relative to the target site of the patient.

Though implant delivery device is depicted and discussed herein as deploying and verifying fixation configurations of and/or for implantable device, it is to be understood that implant delivery device may be used to deploy and verify fixation configurations for other implantable devices or other implantable devices components (e.g., leads of drug delivery devices 1516B) in other examples.

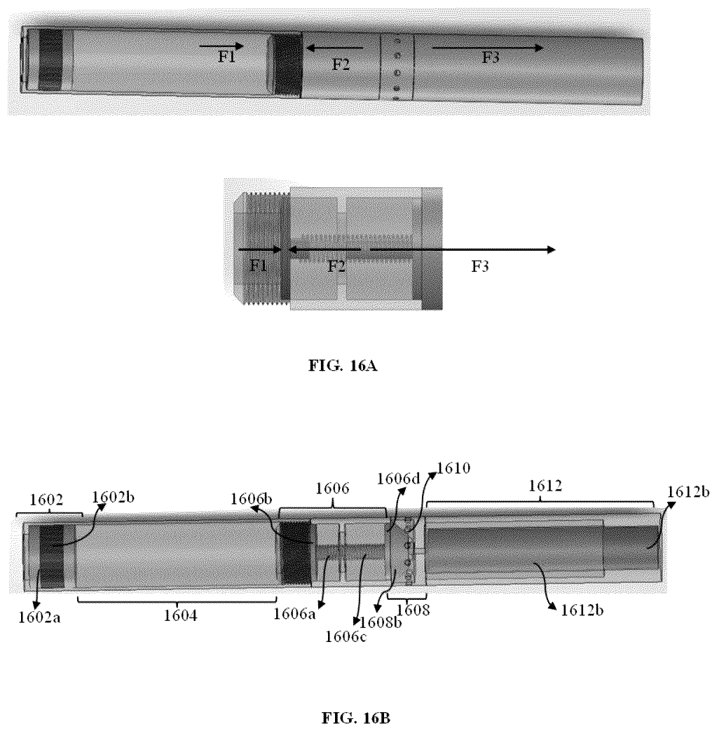

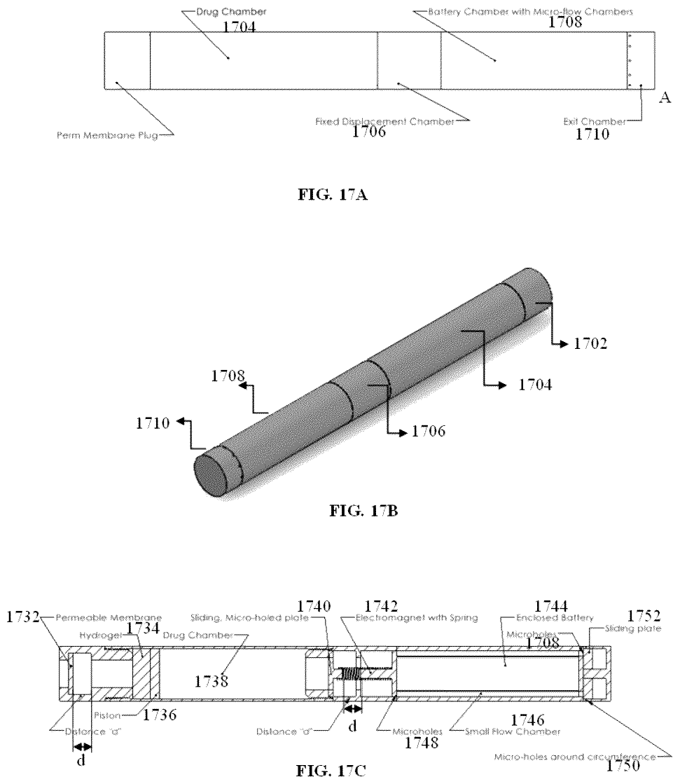

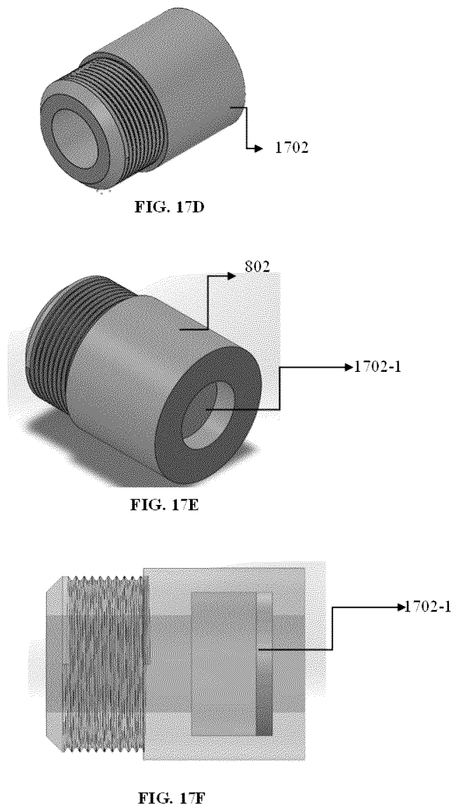

FIG. 16A shows forces in the implantable device to deliver a consistent volume of drug per shot according to one or more embodiments. In an example, the forces can be visualized as F1, F2, and F3 that act in the implanted device to deliver a consistent volume of drug to the animal or the human body, at each shot like that of an injection. F1 is the maximum force exerted on the plate having micro holes for the fluid to flow by hydrostatic pressure within the drug reservoir unit. F2 is the maximum force exerted on the plate by the spring in the fixed-volume chamber. F3 is the maximum force exerted on the solenoid electromagnet by the electronic unit. The force F1, F2, and F3 are such that F1 is less than F2 and F2 is less than F3. F1, F2, F3 may be occurring at different points in time, not necessarily at the same instant.

FIG. 16B shows subassemblies of the implantable device, according to an embodiment. The subassembly of the implantable device comprises an osmotic unit 1602, a drug reservoir unit 1604, a displacement unit 1606, a drug delivery unit 1608, a drug delivery orifice 1610, an electronic unit 1612 comprising an electronic unit 1612a and an insulation cap 1612b. The subassemblies are arranged such that the force F1 is the total force exerted by the drug reservoir unit on the micro holed plate, F2 and F3 are the forces acting from the displacement unit on the micro holed plate. The device works based on osmotic pressure, hydrostatic pressure, and electric pull. In an embodiment, the device size and shape may vary based on the body area where the device is to be implanted. In another embodiment, the size and shape of the implantable device may vary depending on the total dosage or volume of the drug that the device is designed to carry. In an example, the device may comprise one or more pressure sensors that are configured to measure a pressure in one or more chambers of the device.

In an example, the implantable device is a tube-like structure. In another example, the first end of the implantable device comprises an osmotic unit 1602. In another example, the osmotic unit 1602 comprises a semipermeable membrane plug 1602a that allows fluid from the body to flow into the device. Beside the semipermeable membrane, the osmotic unit comprises an osmotic solution 1602b to create pressure and to push the piston forward through the drug unit, letting the drug flow into a displacement unit and a seal plate 1602c to block the flow of osmotic solution to the drug reservoir chamber. The osmotic solution can be one of a hydrogel or a salt solution. In another example, the osmotic solution is a highly concentrated salt solution that results in fluid flow from the body into the implant. As fluid is drawn into the osmotic unit, its volume increases, and this pressure pushes the piston adjacent to the osmotic unit towards the drug reservoir unit. Because of the osmotic pressure exerted on the drug reservoir unit an amount of drug is pushed into the displacement unit 1606 through the micro-holed plate 1606b. In an example, the inner circumference of the drug reservoir unit may comprise a zip tie surface to prevent the movement of the displacement unit 1606 towards the osmotic unit. Whenever a predefined amount of drug is injected into the body of the subject, the piston with the permeable membrane plug will move forward (towards the exit unit) by a certain distance and will not be able to move backwards. The process of non-retractably moving forward continues with each injection till all the drug from the drug reservoir unit is delivered to the subject.

In an example, the implantable device comprises a displacement unit 1606 configured for a fixed displacement. The said fixed displacement is one way to be able to deliver a certain amount of drug in every single injection. The displacement unit comprises a solenoid 1606a and a micro-holed plate 1606b. The micro holes allow flow of the drug when a force is applied from the drug reservoir unit on to the plate. The pumping force acting forward on the microplate by the solenoid enables a constant amount of drug to go into a drug delivery unit 1608. A spring 1606c in the fixed unit will push back the plate comprising micro holes. While the plate comprising micro holes is slowly getting pushed back, the drug from the drug reservoir unit slowly seeps into the displacement unit.

In an example, a drug delivery unit comprises a second plate with micro holes spread across the entire area from where the drug enters the drug delivery unit. The drug delivery unit comprises a valve 1606b and a drug delivery orifice 1610. In an example, the drug delivery orifice 1610 comprises a plurality of fluid exits. In an example, the plurality of fluid exits is radially distributed across the drug delivery unit and the drug can disperse radially from the drug delivery unit to the body of a subject having the device implanted. The number of the exit holes will depend on the structure of the device, the material of the device, quantity of a drug to be delivered, and a rate at which the drug is to be delivered. The valve 1608b is a small check point that allows delivering a drug in the subject's body via a fluid passage 1606d only when a pump pushes on the dose unit comprising the drug. The valve is a one-way valve having low opening force (0.0005-0.5 Newtons), low leakage rates (0-0.01% of rated valve capacity) and provides minimal resistance to flow (0-0.31 psi) in case flow is to be increased. In an example, the one-way check valve is a valve that returns to its initial position after the pressure releases. In an embodiment, the valve generally takes little force to open, approximately 0.00238 Newtons. In some embodiments, the piston (if there is only one) takes much higher at about 0.34 Newtons if using standard rubber seals with direct compression. Indirect compression (bending) takes much less force. In some embodiments, seals such as Teflon may also work, especially since it will only be moving in one direction. A much lower force of around 0.05 might be possible while still maintaining a good seal. A solenoid coil diameter<5 mm provides significant force, but the power demands require changes to the coil wire diameter and turns.

In an example, the one-way check valve is an umbrella valve. The umbrella valve is an elastomeric valve that has a diaphragm shaped sealing disk or an umbrella shape. The umbrella valve allows flow at a predetermined pressure in one way and prevents back flow immediately in the opposite way. When the head pressure on the umbrella valve receives enough force to lift the convex diaphragm from its seat, forward flow occurs. The umbrella valve may be any one-way mechanical fluid valve. The valve should be low leakage. The valve should have a fairly low opening force in order to reduce system power needs.

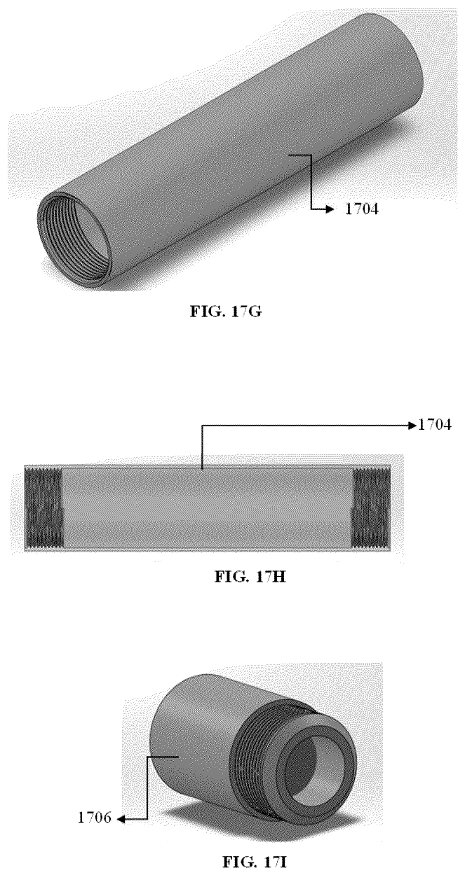

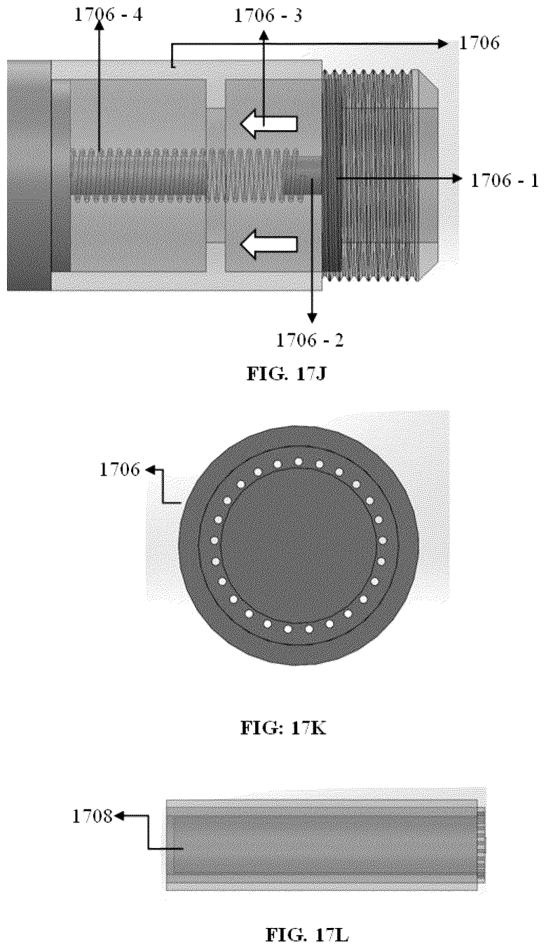

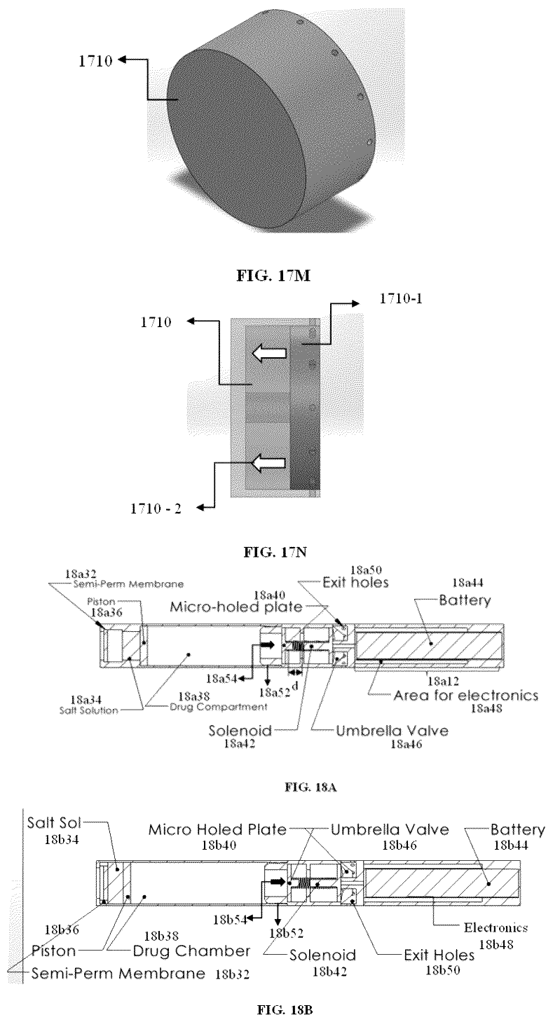

FIG. 17A shows the front view of an implantable device according to an embodiment. The implantable device comprises a semipermeable membrane plug 1702, a drug reservoir unit 1704, a displacement unit 1706, an electronic unit 1708, and a drug reservoir unit 1710. The drug reservoir unit 1704 side is referred to as the front side of the device while the permeable membrane side is referred to as the rear side of the device.

FIG. 17B shows the perspective view of an implantable device according to an embodiment and FIG. 17C shows a cross-sectional view of the implantable device according to an embodiment. The cross section of the device is a longitudinal cross section depicted as line A-A in FIG. 17A. FIG. 17C shows the implantable device or the pump according to an embodiment comprising a permeable membrane plug comprising a permeable membrane 1732, a hydrogel 1734, a piston 1736, a drug reservoir unit 1738; a displacement unit comprising a micro-holed plate 1740 configured for sliding, an electromagnet with a spring 1742; an electronic unit comprising a battery enclosure 1744, a flow unit comprising micro holes around circumference 1750 along the longitudinal direction of the battery connecting the displacement unit and the drug reservoir unit wherein micro-holes 1748 at the displacement unit make the fluid enter the small flow unit 1746 and micro-holes around circumference 1750 of the drug reservoir unit are configured to disperse the fluid into the subject's body in which the implantable device is placed; the drug reservoir unit comprises a sliding plate 1752.

Distance d is the fixed distance that the sliding micro-holed plate must move to displace the drug such that exactly one dosage is administered. Distance d can be calculated based on the requirement for the volume to be dispensed per shot, which is A×d. Volume dispensed per shot is a known volume based on the amount of drug required for one dosage, and A is the surface area of the sliding micro-holed plate. It should be noted that the permeable membrane in the permeable membrane plug is also able to travel this distance d to prevent cavitation.

According to the embodiment, the fixed displacement mechanism is that to make the sliding micro-holed plate 1740 slide forward (in the direction of the drug reservoir unit) an electromagnet with spring 1742 will pull the plate when the electromagnet is powered by a battery. The battery is enclosed near the drug reservoir unit towards the front side of the device. Once the electromagnet with spring 1742 is powered and the drug is administered, the spring will passively move the micro-holed plate 1740 back to its original position. According to an embodiment, the spring is a compression spring, which is an open-coil helical springs wound or constructed to oppose compression along the axis of wind. Meaning, when the electromagnet compresses the spring due to pulling the micro-holed plate 1740 slide forward (in the direction of the drug reservoir unit), the spring will try to move back (in the direction of the permeable membrane) to its initial resting position when the forces from the solenoid are released. When the drug is pushed through the displacement unit, the sliding plate 1752 will move forward, allowing the drug to be dispensed radially through the micro holes around circumference 1750 of the drug reservoir unit. When the spring passively returns to its initial position, then the micro-holed plate moves back to its original position, allowing no backflow to occur whatsoever. The displacement unit 1706 is configured to mimic the process of repeated injections. For example, when the pressure is exerted on the drug reservoir unit 1704 by the semipermeable membrane plug 1702, the drug or fluid is slowly filled into the displacement unit 1706. When the solenoid (or electromagnet with spring) 1742 is activated the drug that is filled in the fixed unit will be pumped into the drug reservoir unit 1704.

FIG. 17D shows a first perspective view of the permeable membrane plug according to an embodiment, FIG. 17E shows a second perspective view of the Permeable Membrane Plug according to an embodiment, and FIG. 17F shows the Transparent side-view of the Permeable Membrane Plug according to an embodiment. As shown in FIGS. 17D, 17E and 17F, the permeable membrane 1702-1 is fixed within the semipermeable membrane plug 1702 and is configured to move distance d. The plug is threaded so that it can be screwed into the back end of the drug unit. The polymeric materials, from which the semipermeable membrane 1702-1 in the plug can be made of, vary based on the pumping rates and device configuration requirements and include, but are not limited to, plasticized cellulosic materials, enhanced polymethylmethacrylate, such as hydroxyethyl methacrylate (HEMA) and elastomeric materials such as polyurethanes and polyamides, polyether-polyamide copolymers, thermoplastic co-polyesters and the like.

FIG. 17G shows the Top view of the 3D model for the Drug reservoir unit according to an embodiment, and FIG. 17H shows the Transparent side-view for the Drug reservoir unit according to an embodiment. The drug reservoir unit 1704, according to an embodiment, has threading on either side. The threading is configured to screw-in the displacement unit from one, and to screw in the permeable membrane plug from the other side. The drug unit, according to an embodiment, is sized to hold a predefined amount of drug. The predefined amount of drug can be configured based on the drug type and the amount of a drug needed for each dosage, number of doses per day, and number of days before a refill.

FIG. 17I shows the perspective view of a displacement unit according to an embodiment, and FIG. 17J shows the transparent side-view of the displacement unit, according to an embodiment. As shown in FIG. 17J, the displacement unit 1706 comprises a micro-holed plate 1706-1 configured to slide in the unit. A drug will pass through the micro holes and fill up the displacement unit 1706. According to an embodiment, the device will be activated by activating the electromagnet 1706-2. The micro-holed sliding plate will move in the direction as shown by the arrows until the two rods touch, preventing any further movement to not overdose. Once the two rods touch, a spring 1706-4 attached to the micro holed plate 1706-1 will passively move the micro-holed sliding plate back to its original position. According to an embodiment, the spring 1706-4 is a compression spring. According to an embodiment, it is a solenoid comprising a coil, piston, and sleeve assembly. In a normally closed valve, a piston return spring holds the piston against the orifice and prevents flow. Once the solenoid coil is energized, the resultant magnetic field raises the piston, enabling flow. When the solenoid coil is energized in a normally open valve, the piston seals off the orifice, which in turn prevents flow. In most flow control applications, it is necessary to start or stop the flow in the circuit to control the fluids in the system. An electronically operated solenoid is usually used for this purpose. Solenoids can be positioned in remote locations and may be conveniently controlled by simple electrical switches. The solenoid is an electromechanical control. A solenoid is an electric coil with a movable ferromagnetic core (piston) in its center. An electric current that runs through the coil then creates a magnetic field. The magnetic field applies a certain force on the piston. Then, the orifice is opened because the piston is drawn towards the center of the coil. When idle, the piston closes off the small orifice. This process controls the flow, allowing the release or cut-off of the fluid flow.

FIG. 17K shows a front view of a micro holed plate of an implantable device, according to an embodiment. The plate comprises micro sized small holes of for a flow of drug from one chamber to another when subjected to a predefined pressure.

FIG. 17L shows a transparent side view of the electronic unit according to an embodiment. The electronic unit 1708 is a unit to hold the battery and electronics. In an embodiment, the electronic unit 1708 around the enclosure of the battery is configured to permit flow of the fluid comprising a drug or a drug combination. The electronic unit ensures that the battery is insulated from contact with any fluid/drug, while also controlling or letting the fluid/drug reach the drug reservoir unit where the drug is radially administered to the patient.

FIG. 17M shows the front view of the electronic unit according to an embodiment, and FIG. 17N shows a transparent side view of the electronic unit according to an embodiment. The drug reservoir unit 1710 is the final chamber in the device. According to an embodiment, the drug reservoir unit 1710 prevents any backflow. When the flow coming from the electronic unit begins, the plate 1710-1 will move along the direction shown by arrows in 1710-2, unblocking the holes placed around the circumference of the drug reservoir unit 1710. According to an embodiment, the number of holes can be 12. The flow will move this plate a distance d until it is not able to move forward anymore, and the drug will be dispensed through these holes. After the drug has fully been administered, a spring, not shown in the figure, will passively return this plate to its original position, once again closing the small holes with the plate 1710-1.

As used herein, “tines” refer to slender pointed projecting extensions of a device which are used as a fixation component. The tines are configured to hold multiple components together during any motion across the device or the movement of the device itself. The tines may include first and second primary tines, having a first length and a second length, respectively, which may be the same as, or different than, one another. The first and second primary tines may be disposed on opposing first curved regions and may be oriented substantially towards each other in the planar configuration. In the planar configuration, the first and second primary tines may at least partially overlap. The tines may also include one or more secondary tines having a length substantially shorter than the first and second lengths of the primary tines. The secondary tines may be disposed on either side of the first and second primary tines.

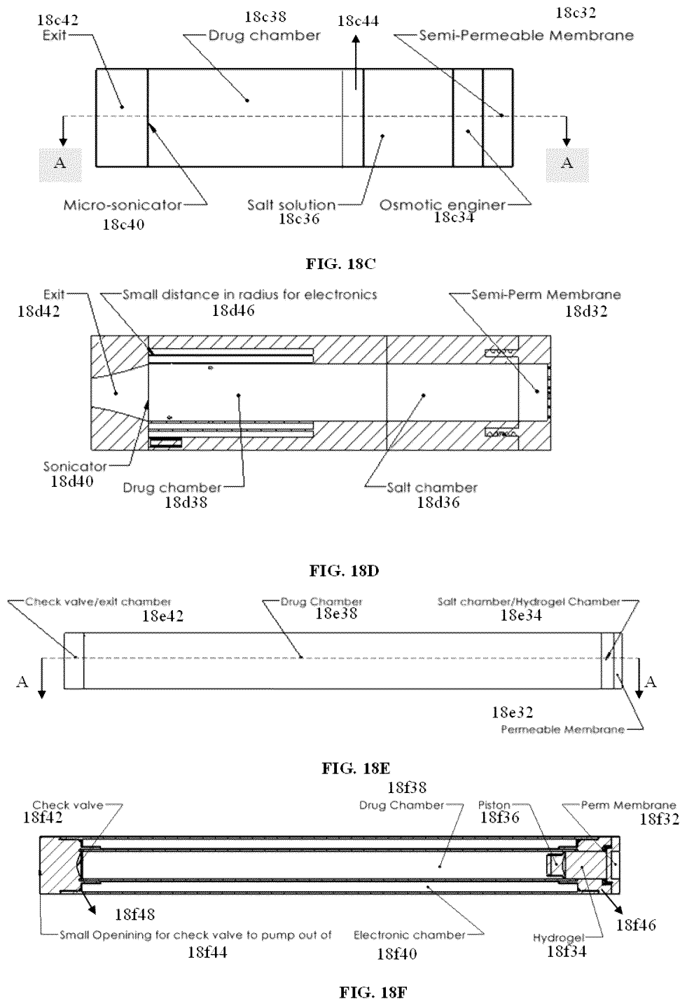

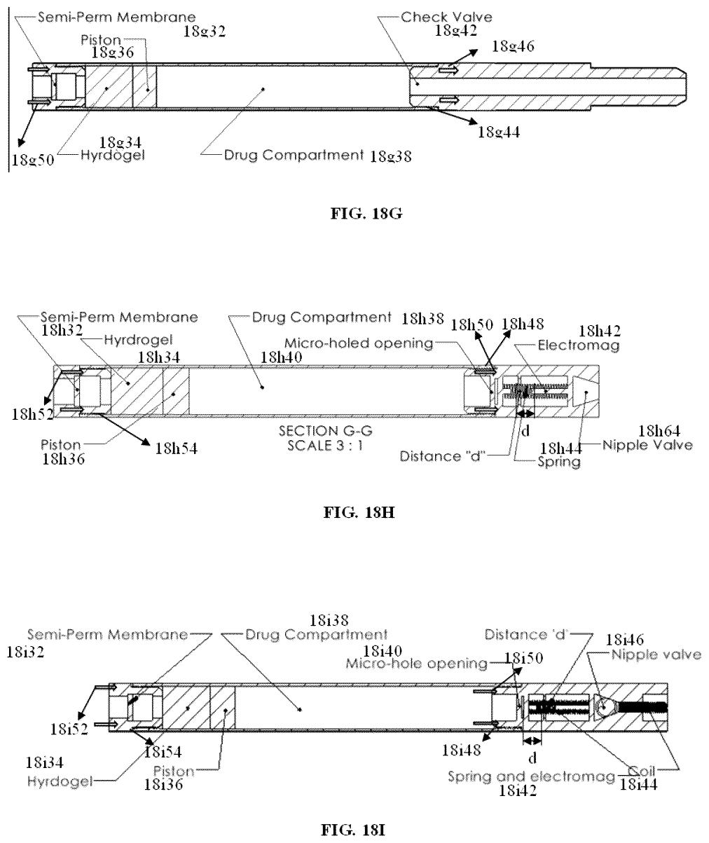

FIG. 18A shows the cross-sectional view of the pump according to an embodiment. The implantable device or the pump according to an embodiment as shown in FIG. 18A comprises a permeable membrane plug comprising a semipermeable membrane 18a32, a hydrogel or a salt solution 18a34, a piston 18a36, a drug reservoir unit 18a38; a displacement unit comprising a micro-holed plate 18a40 configured for sliding, a solenoid 18a42, also referred to as an electromagnet with a spring; an electronic unit 18a12, an umbrella valve 18a46, an area in the electronic unit for electronics 18a48, exit holes 18a50 in the displacement unit. The drug reservoir unit comprises an umbrella valve 18a46 and the exit holes 18a50, wherein the exit holes 18a50 of the drug reservoir unit are configured to disperse the fluid into the subject's body in which the implantable device is placed. In this configuration, the drug reservoir unit, or the unit where the fluid exits, is placed before the battery 18a44 and electronics 18a48. To prevent backflow, the drug reservoir unit houses an umbrella valve 18a46 that is designed to open and close based on a predefined or a pre-set pressure. Once the pressure reaches a predefined pressure level, the umbrella valve 18a46 will open, allowing fluid to go past and out of the exit holes 18a50. Once this pressure falls below the predefined level, the umbrella valve 18a46 will return to its original position, avoiding backflow of the fluid. A zip-tie surface 18a52 around the inner radius of the drug reservoir unit allows the piston to move in the direction shown by the arrow 18a54, but never backwards, i.e., opposite to that of the direction shown by the arrow 18a54.

According to an embodiment, an insulated sphere or tube of radius r around the drug reservoir unit and the electronic unit is configured to allow the electric and electronic connections between the battery and the solenoid. This radius is configured to allow any electrical connections between these two chambers without having any of the electrical connections susceptible to fluid interaction. The functional features or working principle of the implantable device of FIG. 18A are similar to that of FIG. 17C.

FIG. 18B shows the cross-sectional view of the pump according to an embodiment. The implantable device or the pump, according to an embodiment, as shown in FIG. 18B comprises a permeable membrane plug comprising a semipermeable membrane 18b32, a hydrogel or a salt solution 18b34, a piston 18b36, a drug reservoir unit 18b38; a displacement unit comprising a micro-holed plate 18b40 configured for sliding, a solenoid 18b42, also referred to as an electromagnet with a spring; an electronic unit 18b12, an umbrella valve 18b46, an area in the electronic unit for electronics 18b48, exit holes 18b50 in the displacement unit. The drug reservoir unit comprises an umbrella valve 18b46 and the exit holes 18b50, wherein the exit holes 18b50 of the drug reservoir unit are configured to disperse the fluid into the subject's body in which the implantable device is placed. In this configuration, the drug reservoir unit, or the unit where the fluid exits are placed before the battery 18b44 and electronics 18b48. To prevent backflow, the drug reservoir unit houses an umbrella valve 18b46 that is designed to open and close based on a predefined or a pre-set pressure. Once the pressure reaches a predefined pressure level, the umbrella valve 18b46 will open, allowing fluid to go past and out of the exit holes 18b50. Once this pressure falls below the predefined level, the umbrella valve 18b46 will return to its original position, avoiding backflow of the fluid. A zip-tie surface 18b52 around the inner radius of the drug reservoir unit allows the piston to move in the direction shown by the arrow 18b54, but never backwards, i.e., opposite to that of the direction shown by the arrow 18b54.

According to an embodiment, an insulated radius around the drug reservoir unit and the electronic unit is configured to allow the electric and electronic connections between the battery and the solenoid. This small radius is configured to allow any electrical connections between these two chambers without having any of the electrical connections susceptible to fluid interaction. The functional features or working principle of the implantable device of FIG. 18B are similar to those of the FIG. 18A and FIG. 17C.

The device of FIG. 18B is like that of FIG. 18A. The difference is the back end of the device of FIG. 18B, where a permeable membrane plug comprising a semipermeable membrane 18b32, a hydrogel or a salt solution 18b34, a piston 18b36 are coupled together into one piece, i.e., a semipermeable membrane 18b32, a hydrogel or a salt solution 18b34, a piston 18b36 will move forward in tandem, whereas in FIG. 18A where a permeable membrane plug comprising a semipermeable membrane 18a32, a hydrogel or a salt solution 18a34, a piston 18a36 are distinctly separated and hence are configured to move independent of each other. The moving of the semipermeable membrane 18b32, the hydrogel or the salt solution 18b34, the piston 18b36 forward in tandem will provide a constant control over the pressure provided by the salt solution 18b34. The semipermeable membrane 18b32, the hydrogel or the salt solution 18b34, the piston 18b36 will move forward in tandem and then get locked on the zip-tie surface 18b52 around the inner-radius of the drug reservoir unit 18b38.

FIG. 18C shows the front view of the implantable device or pump according to an embodiment, and FIG. 18D shows a transparent side view of the implantable device or pump according to an embodiment. FIG. 18D is the sectional view obtained by taking the longitudinal section at the A-A plane as shown in FIG. 18C. FIG. 18C and FIG. 18D show the implantable device comprises, a semipermeable membrane 18d32, an osmotic engine 18d34, a salt solution unit 18d36, a drug reservoir unit 18d38, a sonicator 18d40, a drug delivery orifice 18d42, a piston 18d44, and an insulated portion around the drug reservoir unit to hold the electronics 18d46. When the pump or implantable device of FIG. 18C is placed under the skin, interstitial fluid moves from the space surrounding the pump through a unidirectional membrane and into an osmotic engine, forcing piston 18c44 movement towards the drug reservoir unit 18c38. In an embodiment, the delivery rate is controlled by the selection of the semipermeable membrane 18c32. In an embodiment, the flow control is configured via the sonicator 18c40. The sonicator 18c40 is powered by the electronics in 18d46.

In an embodiment, ultrasonication can be involved in drug delivery by several mechanisms. The simplest mechanism derives from the oscillatory motion of the insonated fluid, and it can occur in the absence of cavitation. The oscillating fluid increases the effective diffusivity of molecules; thus, the transport of any drug, whether free or bound to a carrier, will be augmented by the oscillatory motion of the fluid. When a strong ultrasonic beam is directed through a partially absorbing liquid, some momentum from the beam is transferred to the fluid, imparting convective motion to the fluid that can also increase the overall rate of drug transport.

FIG. 18E shows the front view of the electronic unit according to an embodiment. FIG. 18F shows a transparent side view of the electronic unit according to an embodiment. FIG. 18F is the sectional view obtained by taking the longitudinal section at the A-A plane as shown in FIG. 18E.

FIG. 18E and FIG. 18F show the implantable device comprises, a semipermeable membrane 18e32/18f32, a hydro gel or a salt solution unit 18e34/18f34, a piston 18f36, a drug reservoir unit 18e38/18f38, an electronic unit 18f40, a check valve 18e42/18f42, and a small opening 18f44 configured for the check valve 18e42/18f42 to pump out the drug into the body of the subjects.

The device comprises inclusion of threading 18f46 in the permeable membrane plug and inclusion of threading 18f48 to promote one way movement of the piston 18f36. Further, the electronic unit 18f40 around the primary flow is provided to hold the electronics needed for the device in an insulated portion. The check valve is not fully depicted in FIG. 18E and FIG. 18F but is configured for the flow control of the drug. A check valve 18f42, also called a one-way valve, is a device that allows the flow of fluids to move only in one direction. The primary purpose of a check valve is to prevent backflow in the system. In an embodiment, the check valve 18f42 can be selected from a spring loaded in-line check valve, a ball check valve, a diaphragm check valve, a swing check valve, a butterfly or wafer check valve, a duckbill check valve.

FIG. 18G shows the cross-sectional view of an implantable device or pump according to an embodiment. The implantable device of FIG. 18G comprises a semipermeable membrane 18g32, a hydro gel or a salt solution unit 18g34, a piston 18g36, a drug reservoir unit 18g38, check valve 18g42. The check valve 18g42 is configured to pump out the drug into the body of the subject. The threaded portion 18g44 will allow the check valve to move in the direction of the arrows 18g46 and not in the direction opposite to the arrows of 18g46. Similarly, the threaded portion 18g44 will allow the piston to move in the direction of the arrows 18g50 and not in the direction opposite to the arrows of 18g50. In this way, cavitation is prevented in the drug reservoir unit 18g38 (or drug compartment) and the salt solution unit 18g34.

FIG. 18H shows the cross-sectional view of an implantable device or pump, according to an embodiment. The implantable device of FIG. 18H comprises, a semipermeable membrane 18h32, a hydro gel or a salt solution unit 18h34, a piston 18h36, a drug reservoir unit 18h38, a micro holed plate 18h40, a solenoid or electromagnet 18h42 with a spring 18h44, and an opening or a nipple valve 18h46 configured to pump out the drug into the body of the subject. The threaded portion 18h48 will allow the check valve to move in the direction of the arrows 18h50 and not in the direction opposite to the arrows of 18h50. Similarly, the threaded portion 18h54 will allow the piston to move in the direction of the arrows 18h52 and not in the direction opposite to the arrows of 18h52. In this way, cavitation is prevented in the drug reservoir unit 18h38 (or drug compartment) and the salt solution unit 18h34. The nipple valve 18h46 is configured for preventing any backflow into the device through the opening of the valve.

FIG. 18I shows the cross-sectional view of an implantable device or pump, according to an embodiment. The implantable device of FIG. 18I comprises, a semipermeable membrane 18i32, a hydro gel or a salt solution unit 18i34, a piston 18i36, a drug reservoir unit 18i38, a micro holed plate 18i40, a solenoid or electromagnet 18i42 with spring, a coil 18i44, and an opening or a nipple valve 18i46 comprising a ball-coil mechanism configured to pump out the drug into the body of the subject. The threaded portion 18i48 will allow the check valve to move in the direction of the arrows 18i50 and not in the direction opposite to the arrows of 18i50. Similarly, the threaded portion 18i54 will allow the piston to move in the direction of the arrows 18i52 and not in the direction opposite to the arrows of 18i52. In this way, cavitation is prevented in the drug reservoir unit 18i38 (or drug compartment) and the salt solution unit 18i34. The nipple valve 18i46 ball-coil mechanism is configured for preventing any backflow into the device through the opening of the valve.

### L. Implantable Device Comprising a Flow Switch

An embodiment relates to an implantable device comprising: a casing that is substantially tubular and has at least a first end and a second end opposite to the first end, a semi-permeable membrane plug at or near the first end, a first chamber, wherein one wall of the first chamber comprises the semi-permeable plug, a second chamber comprising a drug, a piston separating the first chamber and the second chamber, a third chamber comprising a flow switch and an opening for release of the drug from the implantable device into a body of a human or an animal, and a fourth chamber near the second end that comprising electronics.

In an embodiment, the device is configured to deliver medication on-demand, offering improved adherence, and minimizing adverse reactions. The implantable device for drug delivery is configured to address and mitigate the major challenge of non-adherence of diabetic medication. Non-adherence leads to complications and costs billions of dollars in loss to the economy. The implantable device is configured to be implanted within the body of the human or the animal and provide, while implanted within the body of the human or the animal, a constant drug concentration for a certain amount of time, followed by no drug release for some time, and subsequently followed by the constant concentration. Further, the implantable device is configured to produce a desired flow rate of elution of the drug from the implantable device by delivering the drug through the opening via a flow switch. The flow switch is an on-off flow switch.

In an embodiment, the implantable device is implantable without surgery within a human body.

In an embodiment, the device has a dose accuracy of no wider than ±15% of the intended target dose. In another embodiment, the flow discharge accuracy via the flow switch is in the range of ±15%. In another embodiment, the flow discharge accuracy is ±10%. In another embodiment, the flow discharge accuracy is ±5%. In another embodiment, the flow discharge accuracy is ±3%.

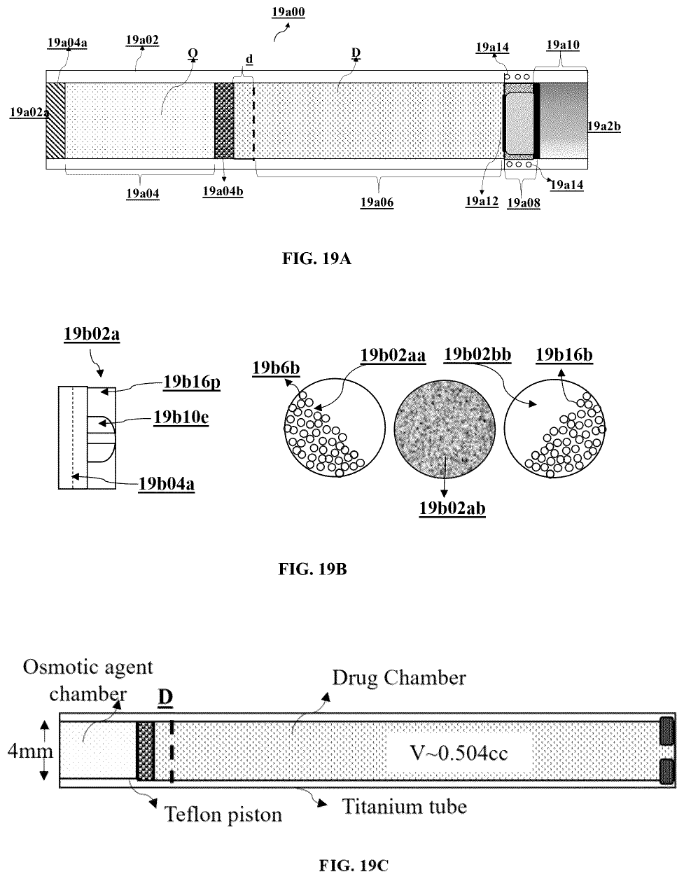

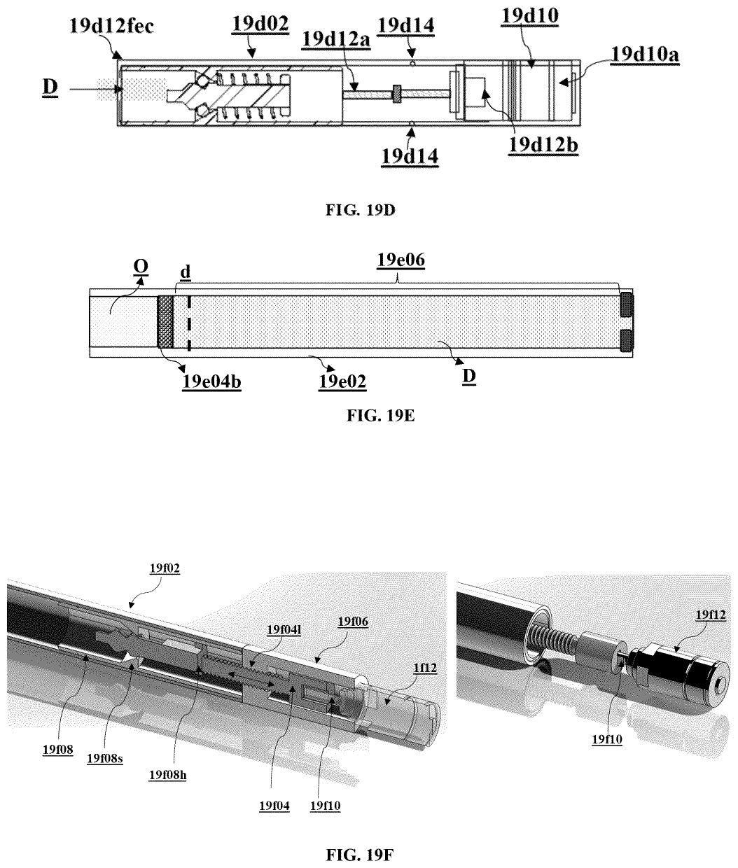

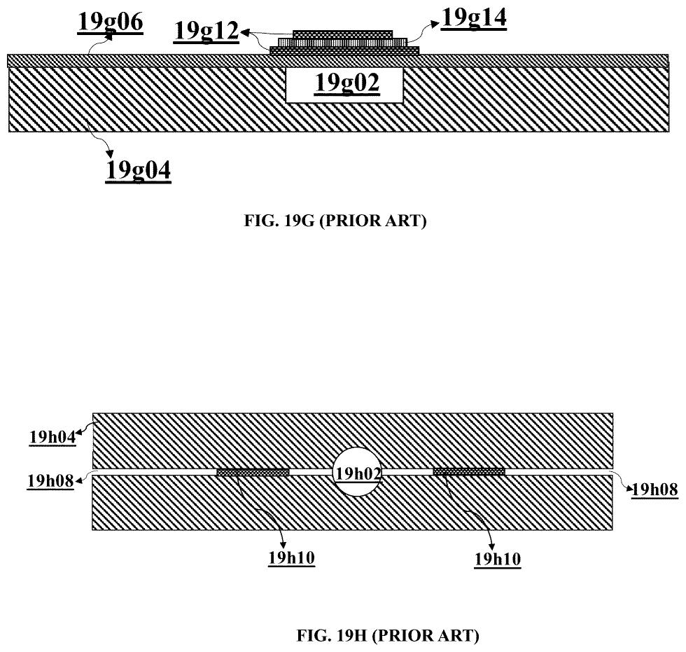

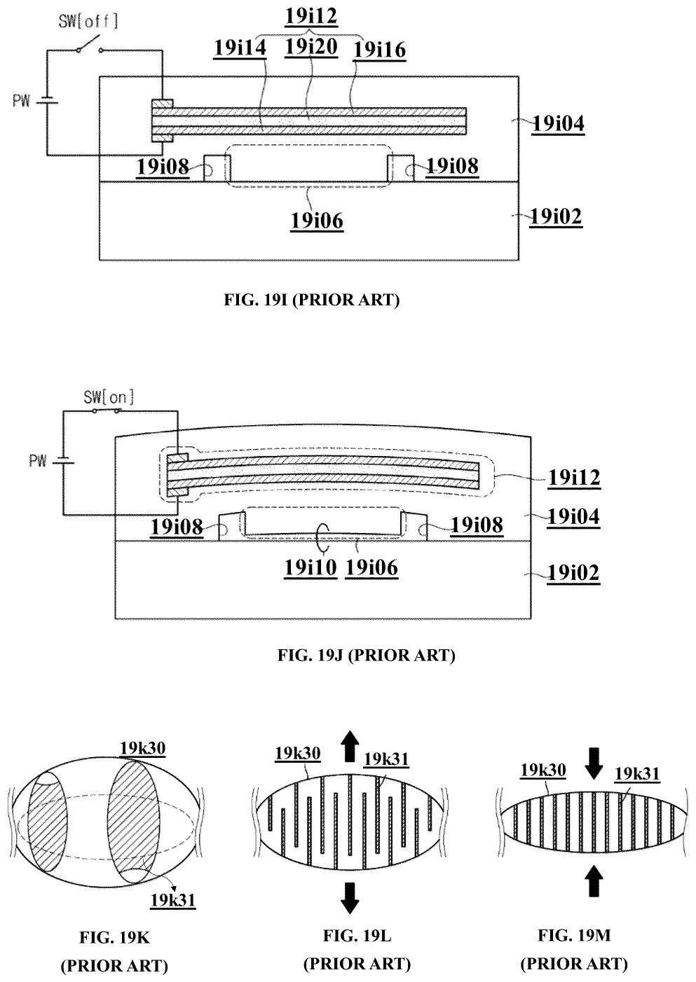

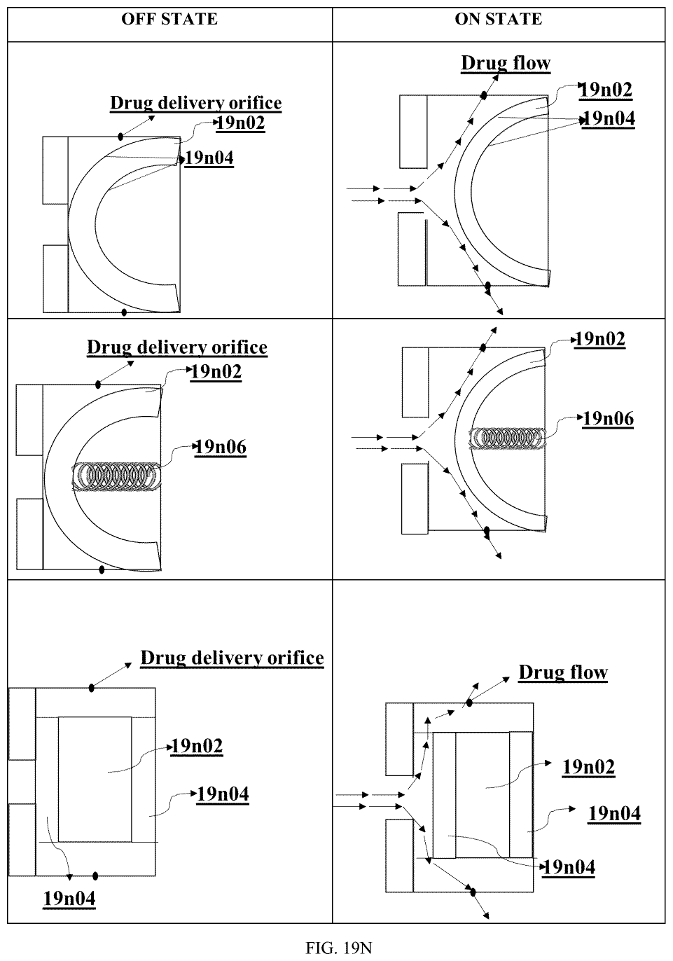

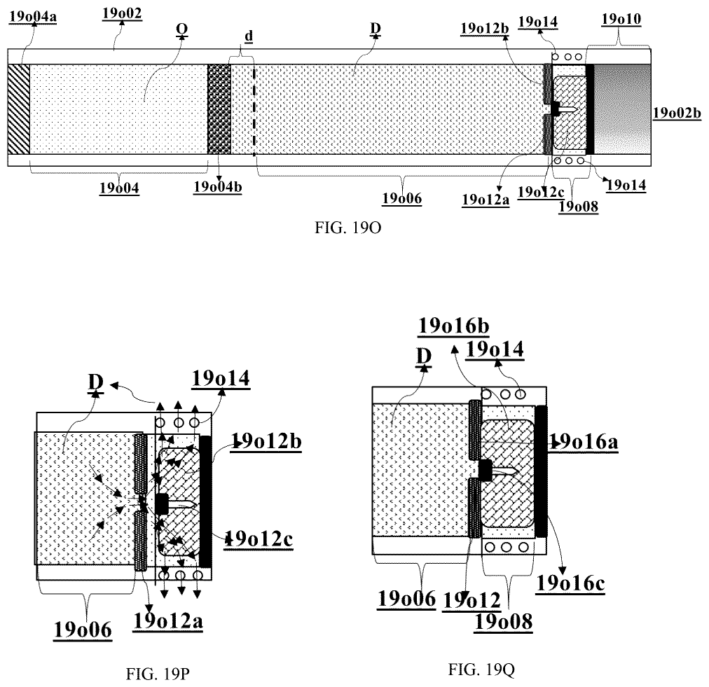

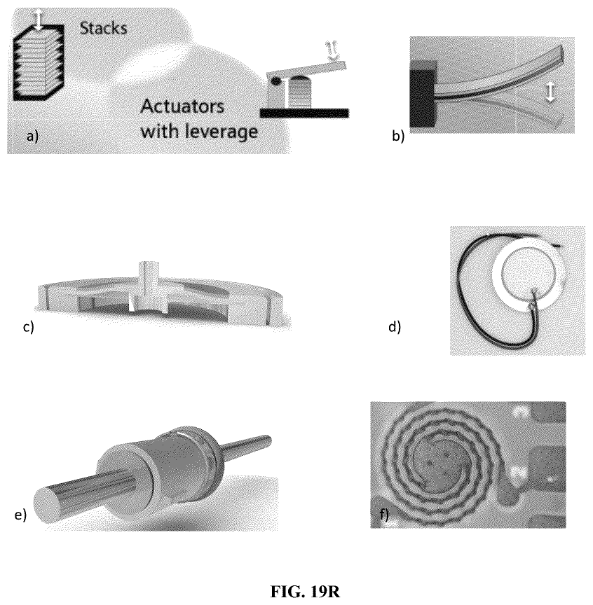

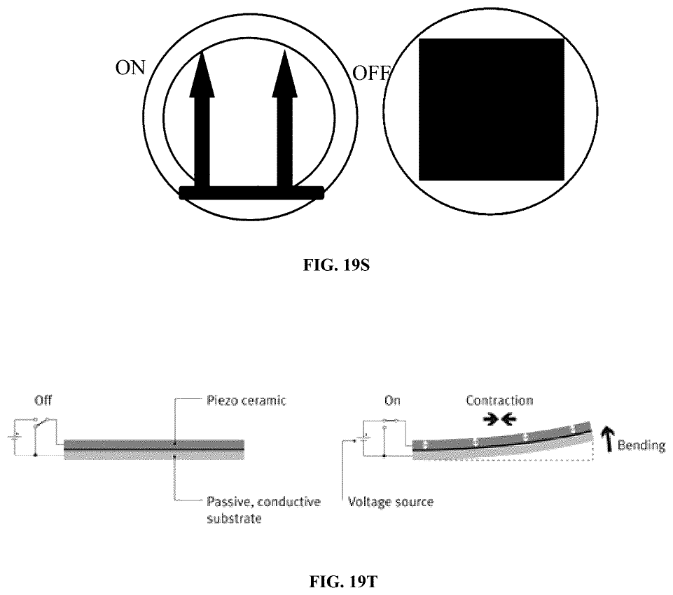

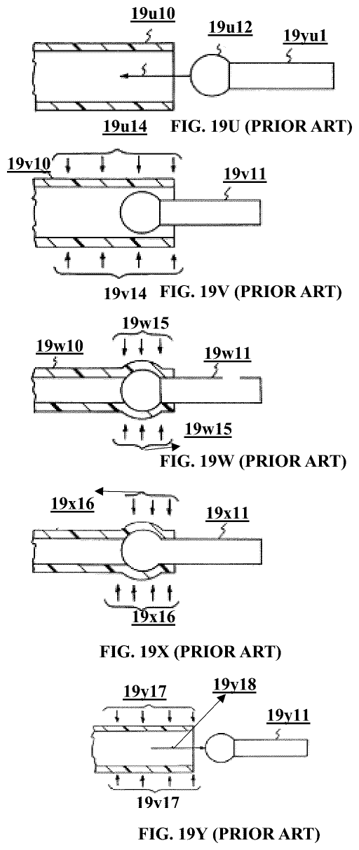

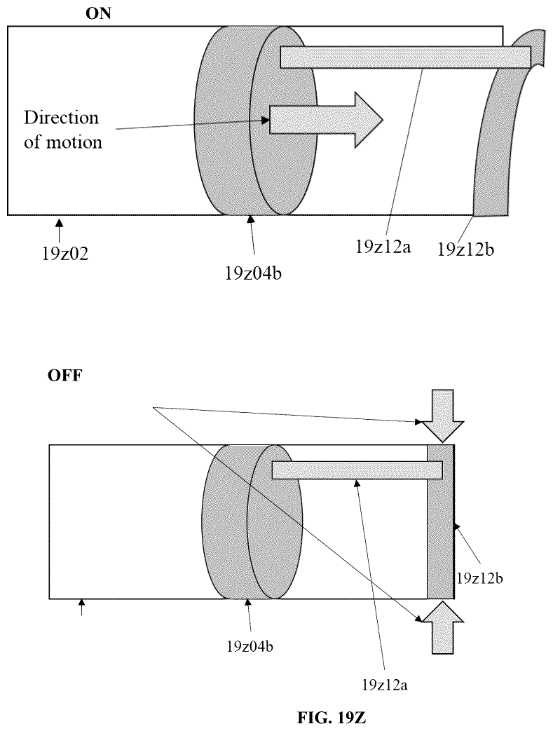

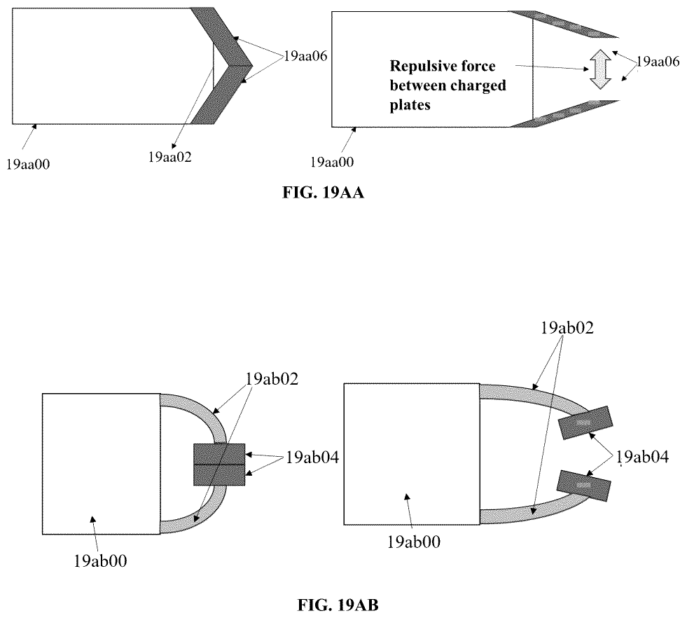

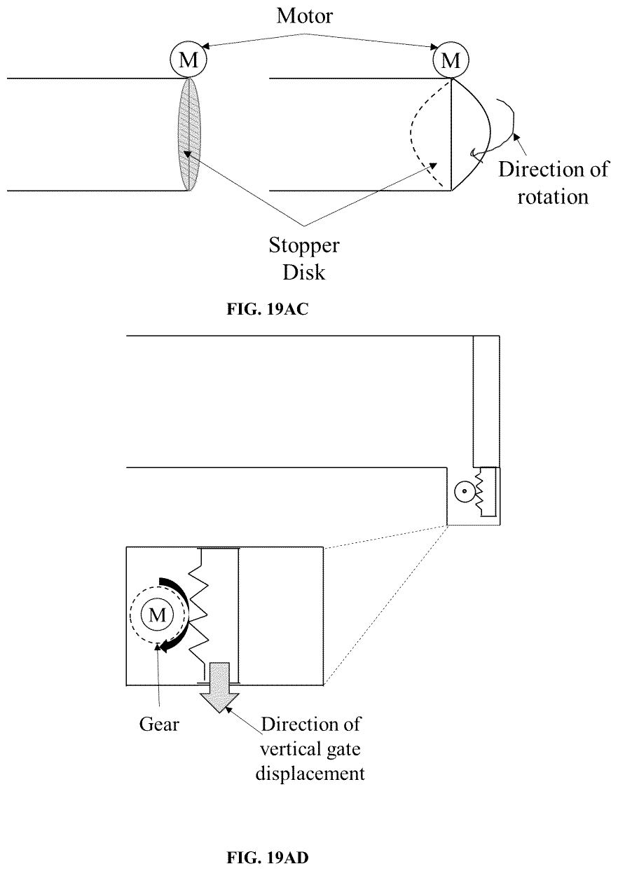

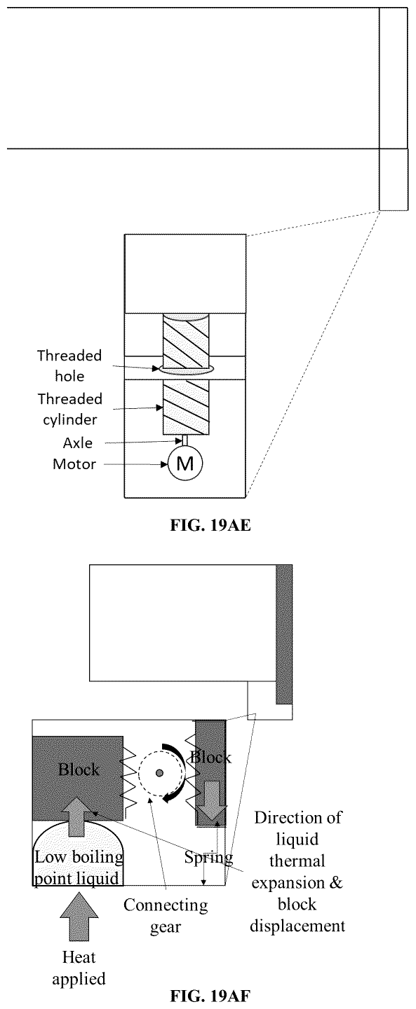

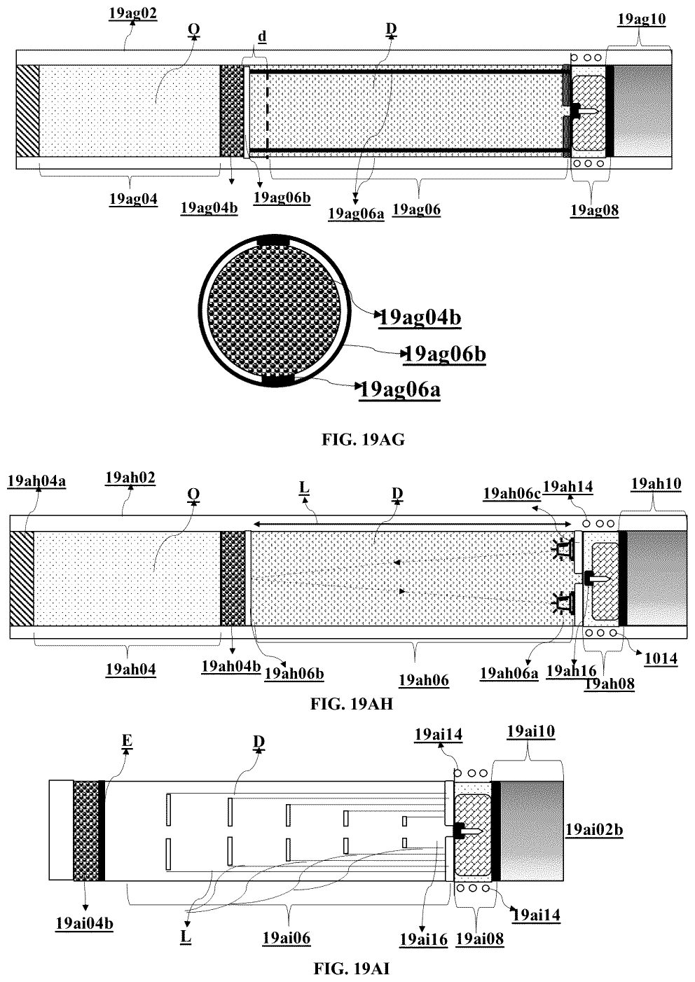

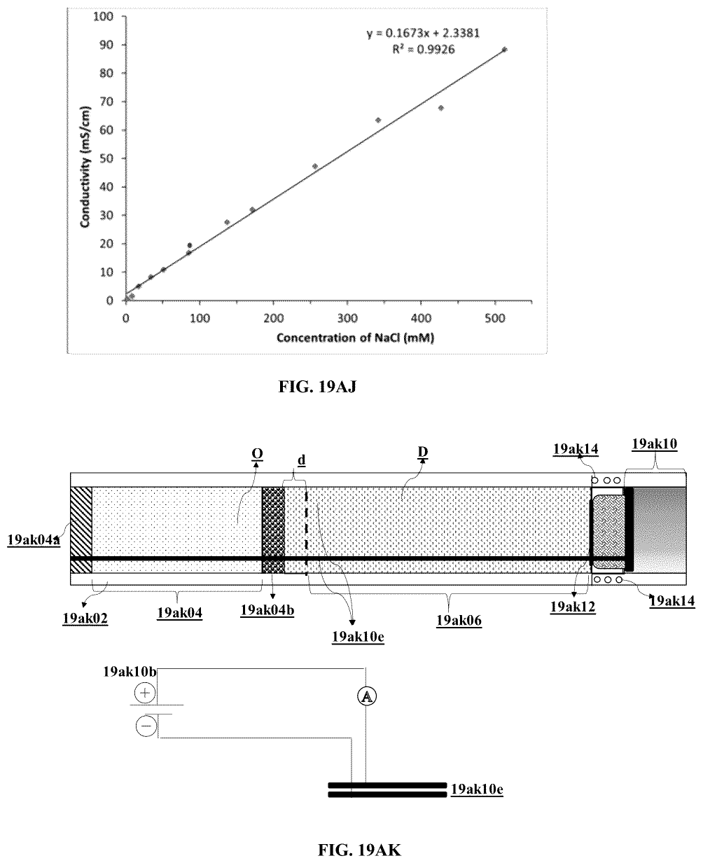

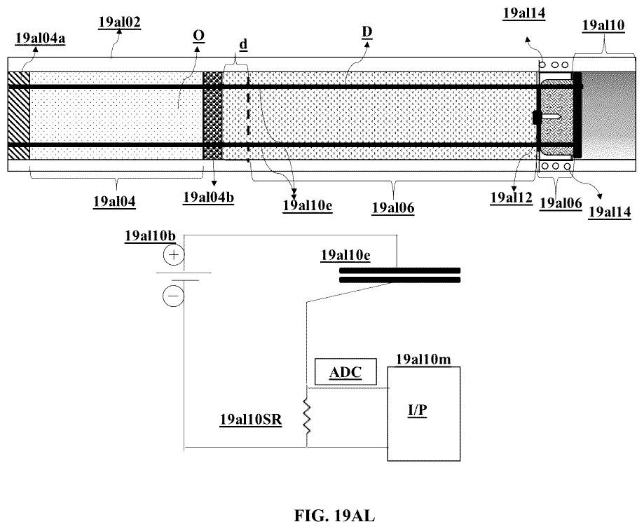

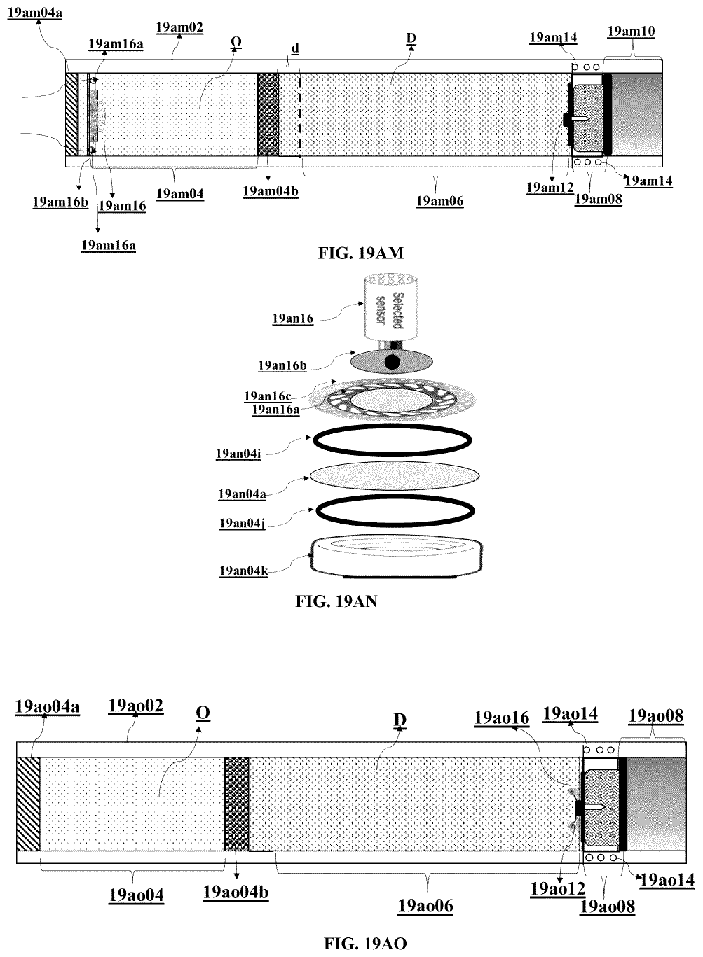

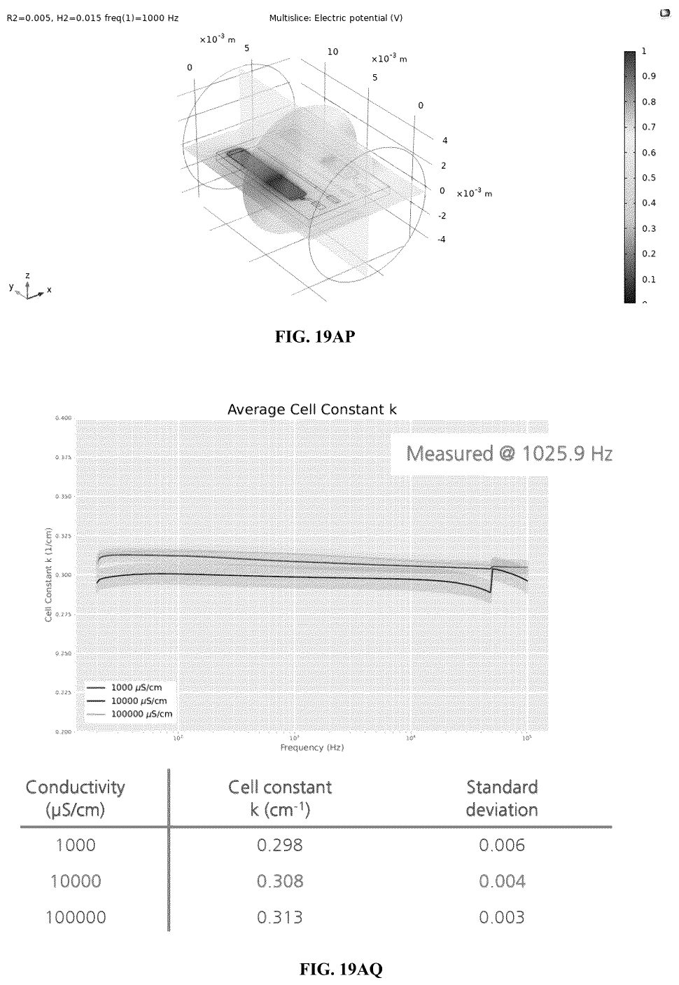

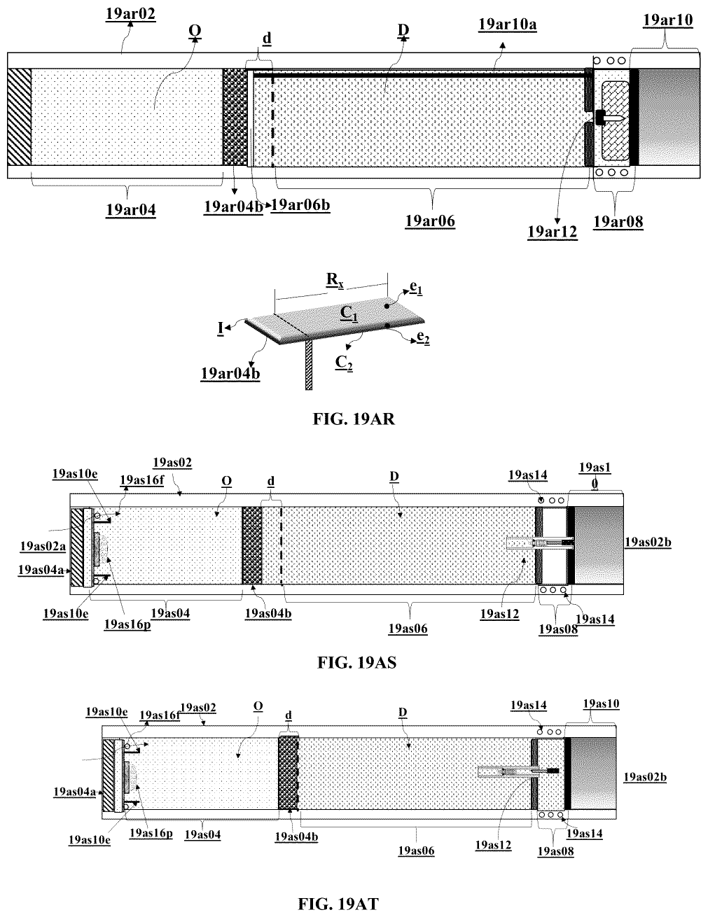

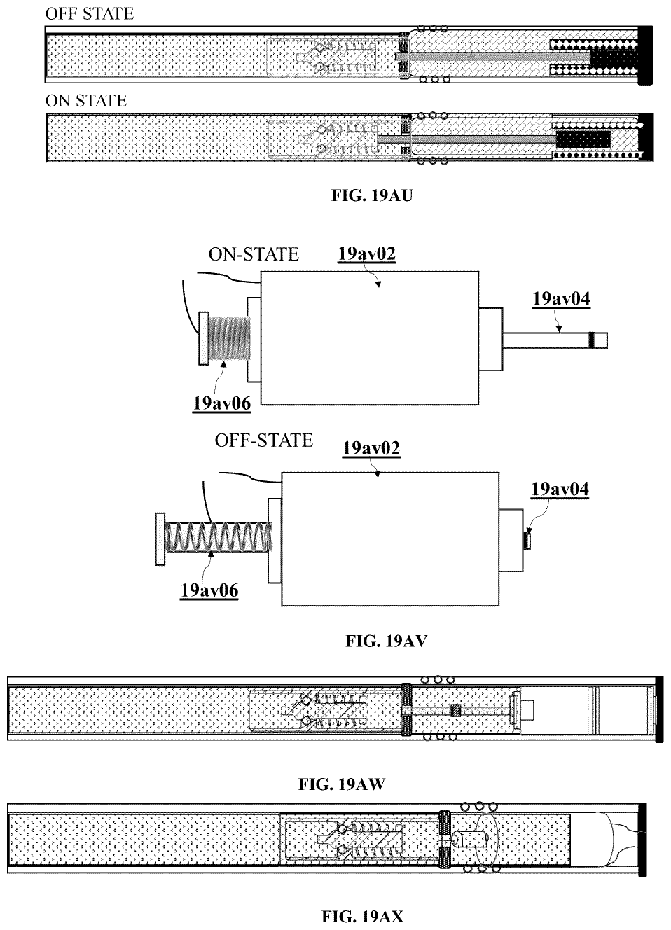

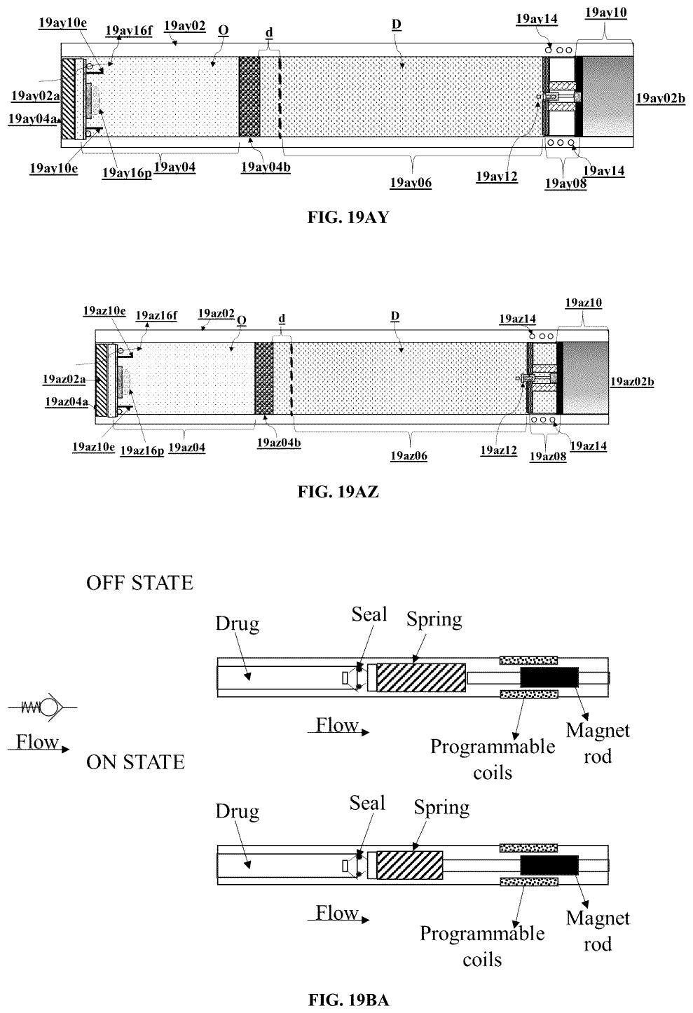

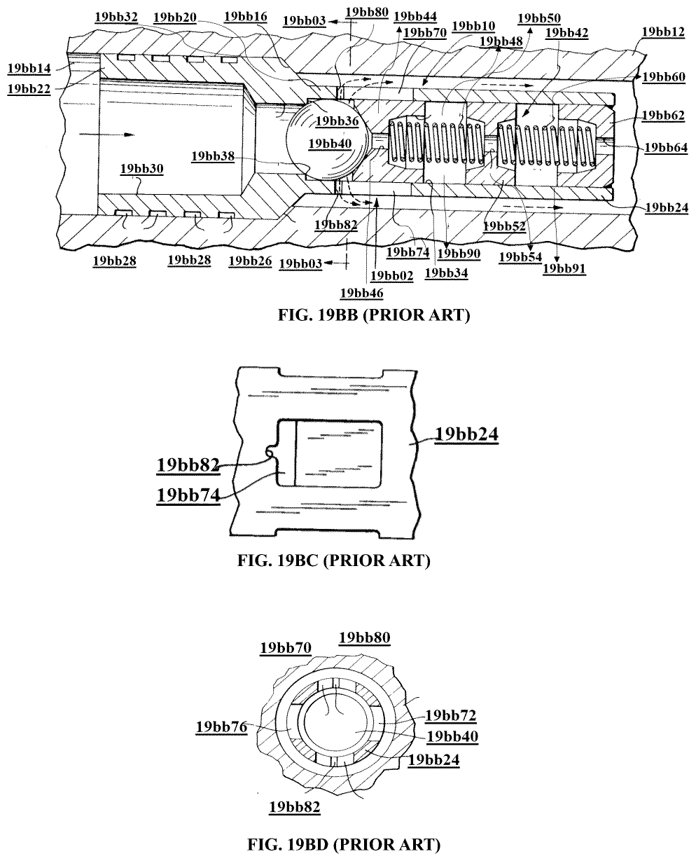

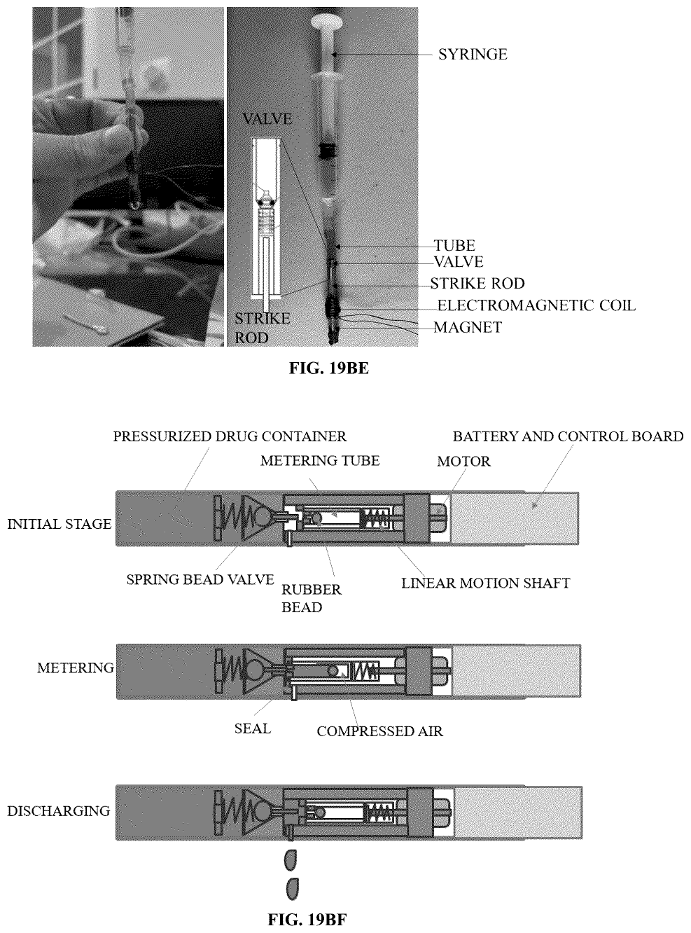

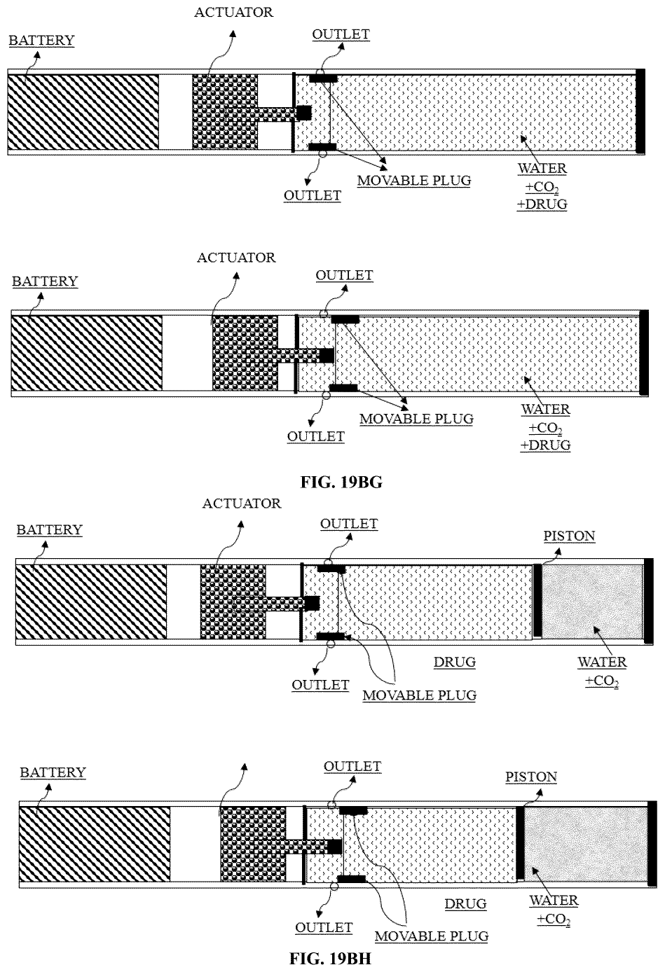

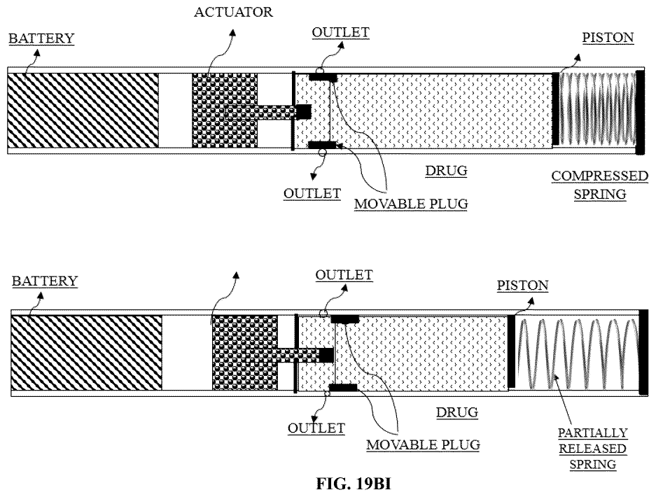

Referring to FIG. 19A, it shows a schematic of the implantable device, according to one or more embodiments. The device 19a00 comprises a casing 19a02 with a first end cap 19a02a comprising a semi permeable membrane plug and a second end cap 19a02 b opposite to the first end cap 19a02a. The device comprises a first chamber 19a04 comprising the semi-permeable plug and a movable piston comprising an osmotic agent O (Salt), a second chamber 19a06 comprising a drug D, a third chamber 19a08 comprising a flow switch and drug delivery orifice and a fourth chamber 19a10 comprising electronics and power supply.

In an embodiment, the first chamber 19a04 is between the semi-permeable membrane plug 19a04a and the piston 19a04b. As liquid (interstitial fluid is drawn into the first chamber 19a04 through the semi-permeable membrane plug 19a04a, the implantable device 19a00 is configured to build an osmotic pressure and push the piston by a distance d). The second chamber 19a06 is between the first chamber 19a04 and the third chamber 19a08. Due to the osmotic pressure exerted via the first chamber 19a04, the piston 19a04b moves towards the second chamber 19a06 and exerts a pressure equivalent to the osmotic pressure. There is an ON/OFF flow switch 19a12 between the second chamber 19a06 and the third chamber 19a08. The ON/OFF switch is operable via the electronics and power supply within the fourth chamber 19a10. When the ON/OFF flow switch is in ON state the drug D gets pushed to the third chamber 19a08 and further to the body of a subject via the drug delivery orifice/s 19a14.

The first end cap serves as the closure for the rear end of the metal tube. It encapsulates the back chamber, which contains the saline solution (or any other fluid) in the implantable device. The first end cap comprises one or more of a threaded design, a snap-fit mechanism, and an O-ring seal. Biocompatible materials such as titanium, stainless steel, or medical-grade plastics can be used as material for the first end cap and the second end cap.

In an embodiment, a front-end cap seals the front end of the metal tube, enclosing the drug suspension chamber. It interfaces with the piston and ensures controlled drug release. Embodiments for the front-end cap comprise one or more of a self-sealing membrane and a pressure relief valve. The front-end cap may be designed with a self-sealing membrane that allows the piston to move while maintaining a barrier between the drug suspension and the external environment. Further, the front-end cap may comprise a pressure relief valve. This prevents excessive pressure buildup within the drug chamber. The front-end cap may further comprise alignment features to ensure proper positioning during assembly.

Osmotic pressure is directly proportional (π) to the salt concentration (C). The final salt concentration (C_{f}) required to facilitate the release of the last dose of the drug from the device will dictate the amount of salt (M) added in the osmotic pump chamber. The maximum operating pressures of the ON/OFF flow switch and the pressure sensor will dictate the maximum allowable osmotic pressure (πₘₓ) in the osmotic pump chamber. The maximum allowable osmotic pressure (πₘₐₓ) will determine the initial salt concentration (Cᵢ) in the osmotic pump chamber. The initial salt concentration (Cᵢ) in the osmotic pump chamber and the amount of salt (M) added in the osmotic pump chamber will determine the initial volume (Vᵢ) of the osmotic pump chamber. Maximum flow rate of the drug suspension is controlled by the maximum ingress rate of the interstitial fluid via the permeable membrane. The maximum ingress rate will occur at the start of operation of the implantable device. The maximum flow rate of the drug suspension will be based on the volume of drug that is needed to be discharged per dose. Assume that the volume of the drug discharged per dose per week is 10 microliters. Then, a membrane having a permeability should be selected such that ingress will take about 10 minutes under the maximum ingress rate and the initial salt concentration (Ci). If the time duration of drug discharge in the subject is at least 10 minutes per dose, then the ON/OFF flow switch can be turned without substantial fluctuations of the flowrate for the drug suspension discharge.

In an embodiment, the ON/OFF flow switch comprises active pumps and valves that are configured for micro dosing of the drug, less power consumption (0 to 20 V, more preferably 0 to 10V, most preferably 0 to 5 volt), and for reinforced durability.

In some embodiments, the device comprises a back-end cap, a cylindrical tube with a piston, a front-end cap and electronics.

In some embodiments, the back-end cap comprises a forward osmosis permeability membrane, at least one of a pressure sensor and a temperature sensor, and a conductivity sensor.

In some embodiments, the front end comprises a stepper motor coupled with a relief valve.

In some embodiments, the electronics comprises a hermetically sealed battery.

Referring to FIG. 19B, the first end cap (or back-end cap) 19b02a contains the permeability membrane 19b02ab sandwiched between two metal plates 19b02aa and 19b02bb with holes (flow channel 19b16b) through one-half of each plate. The ingress flow rate is controlled from zero to maximum by the degree of overlap of the holes in the two plates.

Referring to FIG. 19C, it shows a 4 mm diameter tube with a Teflon piston comprising a drug chamber comprising a drug. The tube is made up of medical grade material approved by United States Food and Drug Administration (FDA or USFDA). In an example the tube is a Titanium tube. The drug in the drug chamber is pushed due to osmotic pressure exerted by the osmotic agent chamber. The piston is operable to move a distance D to deliver a constant volume of the drug via a front-end cap.

Referring to FIG. 19D, a front-end cap 19d12fec comprising a spring-loaded relief valve 19d12rv (like a bicycle tube valve) with a ball and a seat coupled to an actuator 19d12b (stepper motor with helical shaft or vibration motor) with a push rod 19d12a to push the head of the relief valve 19d12rv, thereby creating a slight gap between the ball and the seat of the relief valve. The actuator is powered by a via electronics chamber 19d10 that comprises a battery 19d10a. Adjusting the membrane's permeability or thickness can also impact the flow rate. The arrangement of these holes determines how much liquid can pass through. The liquid ingress can be adjusted by adjusting the alignment of the two metal plates 19d02aa. When the plates are aligned differently, the flow rate changes. When the metal plates 19d02aa and 19d02 bb are aligned such that none of the holes from the first plate overlay the holes in the second plate, the flow rate through the sandwich structure is nearly zero. In this state, the permeable membrane 19d02ab remains effectively blocked, preventing liquid ingress. Conversely, when the plates are aligned such that all the holes in the first plate overlay the holes in the second plate, the flow rate through the sandwich structure is maximum. Liquid can freely pass through the permeable membrane 19d02ab, resulting in unimpeded ingress. By aligning the plates so that partial areas of the holes in the first plate overlay the holes in the second plate, the flow rate falls between nearly zero and the maximum. This intermediate configuration allows fine-tuning of the ingress rate. The pressurized liquid (drug suspension) in the drug chamber is discharged through the gap between the ball and the seat, then through an outlet (holes) in a casing surrounding the front-end cap (the casing is not shown in FIG. 19D) to the exterior of the ID into a human body when the ID is implanted in the human body.

Referring to FIG. 19E, it shows schematic of a tube with a piston, according to one or more embodiments. The diameter of the tube and the volume of the drug chamber may vary based on the subject in which the device is to be implanted, the body part where the device is to be implanted, and the type of the drug that is to be delivered via the device.

Referring to FIG. 19F, the front-end cap assembly comprises an adapter 19f06 accommodating a threaded rod 19f04 and a stepper motor 19f12. The adapter 19f06 may also house the electronics and battery (not shown in FIG. 19F). The threaded rod 19f04 is inserted into the adapter 19f06, securely fastened by mating its threads with those of a precisely engineered threaded hole. Subsequently, the stepper motor 19f12 is inserted into the adapter 19f06, with its rotating arm 19f10 embedded into the slot of the threaded rod. As the stepper motor 19f12 engages, the threaded rod 19f04 rotates in tandem with the motor's rotating arm 19f10. The adapter 19f06 is subsequently joined to the coupler 19f02, which houses the spring-loaded valve 19f08. The valve 19f08 has two pivotal areas, as illustrated in (a) of FIG. 19F. Depending on the rotational direction, the threaded rod 19f04 either extends or retracts into the valve's cavity causing linear movement 19f04m. When the rod extends and applies pressure to the valve head 19f08h, a gap forms between the ball and the valve seat 19f08s. This enables the pressurized liquid (drug suspension) within the drug chamber to discharge through the gap, and subsequently out through outlets on the coupler 19f02 (not depicted). Upon reversing the rotation, the seal to the drug chamber is promptly restored. The front-end cap assembly links to the cylindrical tube and drug chamber via a coupler 19f02. While the connection between the coupler 19f02 and the titanium rod is not depicted, various options exist to finalize this connection as the overall design of the delivery device evolves. Both the coupler 19f02 and adapter 19f06 feature mechanical hard-stops to prevent over-extension of the threaded rod 19f04 in both directions. If viable, consolidating the coupler 19f02 and adapter 19f06 into a single component may be considered.

In an embodiment, the back-end cap of the device is configured to regulate the ingress (inflow) rate of a liquid through the end cap. In some embodiments, the device uses a combination of a mini-osmotic pump with a Teflon piston coupled with an electro-mechanical actuator that controls the release of the material from the reservoir. In some embodiments, the osmotic pump includes a cellulose acetate forward osmosis semipermeable membrane. As the liquids in the device are incompressible, the drug suspension egress rate will be same as the water ingress rate through the permeable membrane.

In some embodiments, the volume of the drug chamber is in the range of 200 μl to 1000 μl. In some embodiments, the volume of the drug chamber is about 500 μl. Assuming 20 μl weekly dose and an ingress/egress rate of 2 μl/sec to 5 μl/sec, then the drug suspension discharge time is 4-10 seconds. If the ingress/egress is 10 times slower, then the drug suspension discharge time is 40-100 seconds. The water ingress rate is controlled by varying the ingress flow area through the back-end cap (FIG. 19B) such that the time required for egress of 20 μl weekly is at least 4 seconds even under the highest initial osmotic pressure. This time duration is sufficient to smoothen out the variations in flow due to the opening and closing of the on-off flow switch. Controlling the ingress flow rate ensures that the rate limiting step for the drug discharge is the ingress rate of water. The on-off flow switch turns on at the appropriate time for dose delivery and turns off when the piston has moved a certain distance. This allows the drug suspension in the drug chamber to flow out of the implantable device through an outlet (holes) in a casing surrounding the front-end cap. The casing surrounding the front-end cap is not shown in FIG. 19D.

To achieve a consistent release of the drug suspension stored in the drug chamber, the osmotic pressure must exceed the friction between the piston and the casing tube.

In an embodiment, the implantable device is made of medical grade materials. The inside and outside walls of the casing tube of the integrated implantable device is coated with an FDA approved epoxy insulation layer. Printed circuits are placed on this epoxy layer by using microprinting technique. The circuit tracks connect sensors and actuators in the drug and osmotic chambers to the electronic chamber. The circuit on the outside surface is coated with an epoxy layer and routed to the electronic chamber through a small hole in the casing tube sealed by epoxy.

In an embodiment, the device comprises a mini-osmotic pump, a piston, and an on-off flow switch. The device uses the mini-osmotic pump with the piston coupled with the “on-off flow switch,” which is a combination of a miniature relief valve and an electro-mechanical actuator, that controls the release of a drug from the device.

In some embodiments, the ON/OFF flow switch is actuated by one of an electroactive polymer actuation, piezoelectric actuation, shape memory alloy actuation, electromagnetic actuation, electrostatic actuation, phase change actuation, Thermopneumatic actuation, magnetic shape memory alloy actuation, rotatory pump, electrolysis-based actuation, and a pump.

### A. Implantable Device Comprising an Electroactive Polymer-Based Actuation ON/OFF Flow Switch

In an embodiment, the device comprises a flow switch comprising electroactive polymer. Electroactive polymers (EAPs) are subjected to dimensional changes when a voltage is applied and are used within the device as a fluid flow restrictor, where the dimensions of a passage for a fluid are changed by applying a voltage across the EAP, such as described in WO13097956 or EP2276163. Alternatively, EAPs are used in a series of linear actuators aligned next to each other to form a peristaltic displacement pumping mechanism, see for example WO08157351. The EAP actuator is a multilayered stack of capacitors comprising a dielectric layer and a conductor wherein the layers are oriented perpendicular to the delivery direction and wherein application of an (actuation) voltage across the multilayered stack of capacitors results in a contraction or elongation along the delivery direction and wherein a voltage modulation, such as ON/OFF switching, sets the driving means in the reciprocating motion with a defined stroke length. The dielectric layer is an elastomer film with a defined and constant film thickness, preferably each layer of the stack has a thickness below 50 micrometer, more preferably below 25 micrometer, even more preferably below 15 micrometer. The dielectric layer is made from a dielectric elastomer such as a silicone, an acrylic elastomer, or a polyurethane. By playing with the number and shape of the stacked layers, the total length and shape of the EAP actuator can be tuned. The conductor layer is typically based on carbon (e.g. carbon black) and is compliant in a plane perpendicular to the delivery direction to accommodate the dimensional changes of the dielectric layers as an actuation voltage is applied. Using the voltage modulation at different frequencies also results in reciprocating movements at different frequencies making the EAP stack a versatile actuator for the drive mechanism. The EAP actuator has end plates at each end of the stack such that a contraction of the EAP stack is effectively transmitted to other parts such as the driving means and retaining means which are preferably directly or indirectly coupled to the end plates. In some embodiments one of the two end plates is operatively coupled to the housing or to the reservoir in the housing. In some embodiments the EAP actuator has an elongated shape with a disc shaped cross section having a central hole enclosing the gear rack along the delivery direction. In some embodiments, the EAP actuator has a C-shaped cross section and at least partially surrounds the gear rack along the delivery direction. The EAP actuators are produced using printing techniques such as disclosed in EP2891194 giving a high degree of freedom in the cross-sectional shape of the EAP actuator.

In an embodiment, the device comprises a flow switch comprising electroactive polymer-based micropump. The micropump described herein features an upwardly open pump chamber introduced into a fluidic substrate made of polymer. This pump chamber is covered by a polymer membrane with an electroactive polymer serving as an actuator. The fluidic substrate is equipped with microchannels for inflow and outflow, displaying nanoscale surface structures that induce direction-dependent flow resistance, resulting in a directed pumping effect. The polymer membrane, forming the upper boundary of the pump chamber, is applied to the fluidic substrate. When a control voltage is applied to the actuator, the electric field generated causes a stretch in the electroactive material, leading to a volume change in the pump chamber. The nanostructured channels further contribute to the valve effect, propelling fluid from inflow to outflow. The polymer membrane, comprising at least one polymer, offers advantages in terms of low material costs and ease of processing. Additionally, the membrane actuator comprises electrodes made of metals, electrically conductive polymers, carbon nanotubes, or their mixtures, ensuring stability within the required temperature ranges. The nanoscale structuring of the microchannels may adopt a sawtooth-shaped pattern with specific amplitude and periodic parameters. The membrane actuator can employ a sandwich arrangement of two electrodes with an electroactive polymer in between. The micropump can be integrated into a microfluidic chip, which, in turn, can be seamlessly incorporated into lab-on-chip systems. This eliminates the need for external pumps, resulting in a compact and miniaturized overall system. The valve effect achieved through nanostructures brings advantages such as the absence of moving valve flaps, reducing wear problems. The nanostructures can be easily introduced into a mold for replication through embossing or injection molding, making the process compatible with standard plastic-based microfluidics. The method for producing the micropump comprises creating a fluidic substrate through embossing, injection molding, or hot stamping in a mold. Microchannels are formed using a mold, and a polymer membrane with electrodes and an electroactive polymer, applied as a sandwich arrangement. The electroactive polymer is applied from the liquid phase. Standard plastics technology processes, digital printing technologies, and heat application methods are employed in this production process. The resulting micropump integrates seamlessly into lab-on-chip systems, marking a significant advantage in terms of compatibility with the overall production process.

Referring to FIG. 19G it shows a cross section through a micropump based ON/OFF switch, according to one or more embodiments. The micro pump has fluid substrate (19g04) that is equipped with a pump chamber (19g02) which is entirely covered by a polymer membrane (19g06) made of polycarbonate. The polymer membrane is actuated by membrane actuators (19g12, 19g14) comprising metals, carbon nanotubes and electrically conductive polymers. The fluid inflow from drug reservoir chamber and outflow via orifice are performed through a micro-channel. Several nanoscale surface structures are provided at specific regions of the micro-channel, such that directed pumping action is achieved by direction-dependent flow resistance.

Referring to FIG. 19H, it shows a top view of a fluidic substrate of the micropump, according to one or more embodiments. The fluidic substrate 19h04, which has microchannels 19h08 with a nanoscale surface structure 19h10. The microchannels 19h08 have an inflow and outflow to the pump chamber 19h02.

In an embodiment, the device comprises a flow switch comprising a micro-valve structure, in which the opening and closing of the valve are directly controlled by a polymer actuator. The micro-valve structure comprises a substrate with a flexible structure, housing a valve portion defining a microchannel. A polymer actuator, featuring a pair of electrodes and an ionic polymer metal composite (e.g., sulfonated tetrafluoroethylene-based fluoropolymer-copolymers), is inserted into the flexible structure. The polymer actuator mechanically controls the microchannel's width by displacing the valve portion. In certain configurations, the microchannel has first and second channels, and the polymer actuator, with a parallelepiped shape, is wider than the sum of the channels and valve portion. The micro-valve structure comprises an inlet, outlet, and a recessed region for the microchannel. The polymer actuator's widest surface can be parallel or perpendicular to the substrate's upper surface. Additionally, the polymer actuator is enclosed by the flexible structure to prevent exposure of electrodes to the external atmosphere or channels. This micro-valve design allows precise control of fluid flow in microfluidic systems.

Referring to FIG. 19I and FIG. 19J, a flexible structure 19j04 is disposed on a substrate 19j02, and a polymer actuator 19j12 is inserted into the flexible structure 19j04. The substrate 19j02 and the flexible structure 19j04 is disposed to define at least one channel 19j08. For example, the channel 19j08 is formed between a bottom surface of the flexible structure 19j04 and an upper surface of the substrate 19j02. More particularly, as shown in FIG. 19I, a sidewall of the channel 19j08 is defined by the flexible structure 19j04. That is, the bottom surface of the flexible structure 19j04 defines the sidewall of the channel 19j08 by recessing upward. However, according to other embodiments, the upper surface of the substrate 19j02 defines the sidewall of the channel 19j08 by recessing downward. The substrate 19j02 is formed of at least one of a polymeric material (example: polyimide, polyethylene, polypropylene, and polydimethylsiloxane), and a biodegradable material (example: polylactic acid and polyglycolic acid polymers) which is an FDA approved material and do not react with a fluid flowing in the channel 19j08 or with materials contained in the fluid. The flexible structure 19j04 is a polymer compound having elasticity. More particularly, the flexible structure 19j04 is a material, which does not react with the fluid flowing in the channel 19j08 or with the materials contained in the fluid among the polymer compounds known as elastomers. For example, the flexible structure 19j04 is formed of polydimethylsiloxane (PDMS). The flexible structure 19j04 having the channel 19j08 is formed using a soft-lithography technology. For example, the channel 19j08 is formed on one surface of the flexible structure 19j04 by using one of micro contact printing (μCP), replica molding (REM), microtransfer molding (μTM), micromolding in capillaries (MIMIC) or solvent-assisted micromolding (SAMIM) technologies. The flexible structure 19j04 is adhered onto the substrate 19j02 through a bonding process like an oxygen plasma treatment.

According to some embodiments, the flexible structure 19j04 comprises a valve portion 19j06 disposed between the channels 19j08, and the sidewalls of the channel 19j08 is defined by the valve portion 19j06. The bottom surface of the valve portion 306 is substantially in contact with the upper surface of the substrate 19j02, but these surfaces may not be adhered to each other. Therefore, as illustrated in FIG. 19J, the distance between the valve portion 19j06 and the substrate 19j02 is controlled by the polymer actuator 19j12. The polymer actuator 19j12 comprises an electrically separated pair of electrodes 19j14 and 19j16 and an electroactive polymer 19j20 disposed between these electrodes 19j14 and 19j16. In some embodiments, the electrodes 19j14 and 19j16 comprise at least one FDA approved metallic material. For example, the electrodes 19j14 and 19j16 are platinum or gold, which is coated on two surfaces of the electroactive polymer 19j20 facing each other. According to some embodiment, the electrodes 19j14 and 19j16 of the polymer actuator 19j12 are not exposed by an external atmosphere or the channels 19j08. For this purpose, a thin protective layer (not shown) is further formed on a surface of the polymer actuator 19j12. In some embodiments, the protective layer has a flexible characteristic. The electroactive polymer 19j20 is a material exhibiting a bending actuation under an applied voltage. For example, the electroactive polymer 19j20 is an ionic polymer metal composite (IPMC). When the ionic polymer metal composite is used as the electroactive polymer 19j20, the potential difference between the electrodes 19j14 and 19j16 generates the foregoing bending actuation and the accompanying displacements of the polymer actuator 19j12 and the valve portion 19j06 by means of ion migration and electrostatic repulsion generated in the ionic polymer metal composite. According to some embodiments, the ionic polymer metal composite is a sulfonated tetrafluoroethylene-based fluoropolymer-copolymer. According to some embodiments, the ionic polymer metal composite further comprises graphene oxide or graphene. The polymer actuator 19j12 is formed adjacent to the valve portion 19j06 in the flexible structure 19j04. In this case, as illustrated in FIG. 19J, when the voltage potential difference between the electrodes 19j14 and 19j16 is generated, the valve portion 19j06 is spaced apart from the substrate 19j02 due to the bending actuation of the polymer actuator 19j12. As a result, a microchannel 19j10 connecting between the channels 19j08 is formed between the valve portion 19j06 and the substrate 19j02.

According to some embodiments, as illustrated in FIG. 19I and FIG. 19J, the valve portion 19i06 is mechanically and directly connected to the polymer actuator 19i12. Accordingly, the valve portion 19i06 is directly actuated by the polymer actuator 19i12, by the voltage potential difference/s between the electrodes 19i14 and 19i16. As a result, the mechanical displacement of the valve portion 19i06 is directly controlled by the polymer actuator 19i12. The foregoing configurations may provide far superior characteristics in terms of a reaction speed and an actuating force as compared to the modified embodiments in which the valve portion 19i06 is spaced apart from the polymer actuator 19i12. According to the foregoing embodiments, the polymer actuator 19i12 is formed to have a width greater than the sum of widths of the pair of the channels 19i08 and the valve portion 19i06.

In one embodiment, the ON/OFF flow switch comprises an electroactive polymer-based micropump (EAP micropump) with a configuration of multiple diaphragms. In accordance with this embodiment, illustrated in FIG. 19K, FIG. 19L, and FIG. 19M, a drug movement passage is established between one end of each of the multiple diaphragms 19k31 and the inner wall of the micropump chamber 19k30. Adjacent passages in the micropump chamber, based on the central axis of 19k30, is alternately oriented in opposite directions.

In FIG. 19K, each diaphragm 19k31 is shaped along the inner circumferential surface of the chamber 19k30. The size of these diaphragms 19k31 may vary based on the internal structure of the chamber 19k30. For instance, if the chamber 19k30 has a rugby ball shape, the diaphragms 19k31 is smaller as they move away from the center of the chamber 19k30. In this scenario, one end of each diaphragm 19k31 is not formed along the inner circumferential surface, creating an empty space that serves as a drug movement passage.

In FIG. 19L, adjacent passages in the chamber 19k30 are alternately oriented in opposite directions along a horizontal axis. This configuration prevents drug leakage through the drug injection passage while maintaining a consistent drug dose inside the chamber 19k30. When the EAP micropump 19k20 is relaxed, the zigzag passage formed by the diaphragms 19k31 opens, allowing the drug to be accommodated in the empty space inside the chamber 19k30 without leaking.

In FIG. 19M, when the EAP micropump contracts and the chamber 19m30 shrinks, the zigzag passage is blocked, causing the drug in the empty space to move in the direction of drug injection. In the contracted state, the drug cannot move to the empty space between the diaphragms 19m31. Upon expansion of the chamber 19m30, as shown in FIG. 19M, the empty space inside is available again. This arrangement ensures a constant drug dosage per contraction-relaxation cycle of the EAP micropump, preventing drug leakage into the regulator or the body from the chamber 19m30 and effectively administering the drug dosage.

In an example, the ON/OFF flow switch is based on the working principles of NEXIPAL® actuator. The NEXIPAL® actuator utilizes the electroactive properties of silicone film laminates to function as a drive system component. Comprising at least one ultrathin silicone film (ELASTOSIL® film) and two electrodes, the NEXIPAL® laminate serves as an actuator when a voltage source is applied. Turning on the voltage source charges one electrode positively and the other negatively, causing the electrodes to attract each other and deform the silicone film. Despite silicone's virtually incompressible nature, the volume remains constant as the film becomes thinner yet wider. When the voltage is turned off, the silicone film returns to its original state, pushing the electrodes apart again. This mechanism allows the NEXIPAL® actuator to convert electrical signals into controlled mechanical movement, making it suitable for various applications in actuation and sensing systems.

Referring to FIG. 19N, shows various illustrations of NEXIPAL® actuator arrangements to be used in flow switch in on and off states, according to one or more embodiments. NEXIPAL® comprises laminated electroactive polymer 19n02 sandwiched between two electrodes 19n04. In some versions, the NEXIPAL® laminate switch further comprises a spring (19n06) to help the electroactive polymers quickly return to their “off” state after being switched “on.” In some embodiments the flow switch comprises a thick, flat electroactive polymer 19n02 sandwiched between two electrodes 19n04. The flow switch turns on and off, opens and closes, because the electroactive polymers shrink when an electric field is applied and then expand back to their original state when the field is removed.

### Implantable Device Comprising Electromagnetic Flow Switch

In an embodiment, the flow switch comprises an electromagnetic (EM) switch in conjunction with a rigid diaphragm that has a hole in the center. This switch separates the second chamber comprising the drug suspension from the third chamber. In its normal state, OFF state, the switch rests firmly on the diaphragm, closing the passage. The controller, under the command of a CPU, energizes the electromagnetic switch, causing it to be pulled back, opening the hole. This action allows the drug suspension to flow out of the device through the holes in the wall. When the controller de-energizes the switch, it moves and rests on the diaphragm, thereby closing the passage.

In an embodiment, the frequency of the signal applied to the EM switch varies around the resonance frequency of the third chamber and the measured back electromagnetic field (BEMF). A maximum BEMF is sensed at the resonant frequency. The length L of the drug chamber can be directly computed as follows:

$$f = \frac{c}{2 L} \qquad Equation 1$$

where f is the resonant frequency of sound in a tube of length L closed at both ends and c is the speed of sound. Nominal speed of sound in air at normal atmospheric pressure and 25 deg C. is about 243 meters/sec. The speed of the sound would be higher in the third chamber comprising the drug. As the piston moves, the frequency will change with the changing L. By measuring the frequency, the length can be determined by the microprocessor.

Referring to FIG. 19O, it shows the device comprising an electromagnetic switch 19o12a. The electromagnetic switch 612a is in conjunction with a rigid diaphragm 619oa and separates the second chamber 19o06 comprising the drug suspension D from the third chamber 19o08. The controller 19o12c controls the ON/OFF state of the switch based on a command received by a microprocessor present in the fourth chamber 19o10. The electromagnetic switch 19o12a is integrated into the fluid path of the drug from the second chamber 19o06 to the third chamber 19o08 (therefore through the drug delivery orifice 19o14) and incorporates a mechanism that can control the flow of the drug suspension D. The electromagnetic switch 19o12a utilizes an actuator and a flow control mechanism, influenced by an electromagnetic coil. The switch is designed to provide instantaneous and reliable transitions between allowing and restricting drug suspension D flow, with no backflow when transitioning to the “off” state.

Referring to FIG. 19P, it shows the electromagnetic switch 19p12a in ON state. An actuator, responsive to electromagnetic forces, is positioned within the switch. This actuator is designed to actuate a valve or a similar flow control mechanism. An electromagnetic coil is placed near the actuator. When energized, the coil generates a magnetic field. In the “on” state, the electromagnetic coil is energized by the controller following a command from the microprocessor.

The generated magnetic field influences the actuator, causing it to allow the flow control mechanism to open, permitting the drug suspension to flow through the electromagnetic switch to a body of the subject's body via the third chamber 19p08 through the drug delivery orifice 19p14. The design ensures that there are no obstructive components in the fluid path when in the “on” state, allowing for smooth fluid flow.

Referring to FIG. 19Q, it shows the electromagnetic switch 19ql2a in OFF state. When the microprocessor sends a command to turn the flow switch to the “off” state, the controller de-energizes the electromagnetic coil. The absence of the magnetic field allows the actuator to respond by closing the flow control mechanism, effectively restricting, or blocking the drug suspension D flow path. In the “off” state, the switch mechanically restricts the flow path, preventing or significantly impeding the movement of the drug suspension D. The design ensures an instantaneous transition between the “on” and “off” states. There are no moving parts obstructing the flow path in the “on” state, minimizing the possibility of backflow when transitioning to the “off” state. The actuator design and the responsiveness of the flow control mechanism contribute to the rapid and reliable switching between states.

### B. Implantable Device Comprising a Piezoelectric ON/OFF Flow Switch

In an embodiment, piezo elements can be used to actuate the ON/OFF switch for controlled drug delivery from the second chamber to the third chamber.

In some examples, piezo-actuated micropumps can be used for microdosing, wherein the pump membrane is actuated by means of a piezo element. For example, if a disc-shaped piezo membrane actuator is applied to a membrane, a lateral contraction takes place by applying a positive electric field, which leads to a downward deflection of the membrane. When applying a negative voltage, however, the membrane actuator curves upwards. Due to piezo physics, in contrast to the positive voltage, only a relatively low negative voltage can be applied before the piezo repolarizes. Thus, in particular in the downward movement direction, the actuator is advantageously used in a technically efficient manner. This is particularly important in the miniaturization of a normally closed microvalve, since piezo membrane actuators only have a vertical stroke of a few micrometers, and a high flow rate is often needed in the open state. In addition, this stroke is reduced by bias pressures or fluid pressures that are present.

In an embodiment, the ON/OFF flow switch comprises a piezoelectric actuator. The piezoelectric actuator is one of a Piezo Walker, Stick Slip Piezo, disc actuator, piezo valve, bimorph bender, flapper or the like. For some embodiments, piezo walker and stick slip piezo have shown immense potential in moving shuttles for the drug delivery device.

Referring to FIG. 19R, it shows examples of piezo elements that can be used in the flow switch of the device for ON/OFF actuation, according to one or more embodiments.

When a current is supplied on stacked piezo elements (a), piezoelectric crystal changes in one of the axis (for example, YZ axis. These crystals, when stacked together, can provide motion. Piezo stack with a rubber fitting can be put on the top of the ON/OFF switch opening towards the drug chamber. When the stack stretches, on current flows, it closes the switch. When the current stops, it relaxes and opens the switch.

When current is provided to piezo bender (b), it flexes. The potential use would be for a switch to close and open. So, when current is provided, motion of the piezo bender opens the flow switch and when current is stopped the flow switch is closed by the benders. It can have a small hole. This normally is in the closed position or down position. When it's off, it plugs the hole. There can be a rubber fitting on the bottom of the bender.

The stick slip piezo (d) comprises a slider, a stator and a moving platform and allows movement of a rod by using the piezo element. At time T=t₀, the slider and the driving foot (a small platform attached to the moving platform) are in contact due to an applied preload force.

When a sawtooth waveform signal is applied to the piezo stack within the moving platform. This causes the stack to elongate and contract rapidly. This rapid movement creates two crucial displacements: Lateral displacement and Longitudinal displacement. The stretching and contracting of the piezo stack push the driving foot sideways, propelling the slider along its track. The piezo stack's elongation also increases the pressure between the slider and the driving foot, ensuring secure contact and efficient power transfer. This cycle of stick (pre-loaded contact) and slip (lateral movement with increased pressure) repeats with each sawtooth cycle, ultimately translating the precise piezo actuation into controlled and reliable movement of the slider.

Thermoelastic spiral bimorph actuator (f), constructed from three spiral arms with stiffeners supporting a central rigid stage, is subjected to a slow square-wave voltage, and performs a piston motion (parallel out-of-plane).

The disc actuator (c) and (d), when flexed concave and convex based on voltage, can effectively serve as both a pump and a valve. The pump function propels pharmaceutical fluids, while the valve function controls the flow, enabling accurate dosage control. Bimorph benders, with their flexing arms, can effectively act as flapper valves. This motion can regulate fluid flow and ensure that medications are delivered with precision.

In an embodiment, the ON/OFF flow switch comprises a piezo bender-based switch. In some embodiments, two piezoelectric ceramic plates are bonded together with a supporting material and subjected to opposing electric fields. The contraction of the ceramic when the operating voltage is applied results in deflection and force on the tip of the bending actuator. Or, if a force is applied to the tip, this generates an electrical charge. This differential actuation induces bending deformation in the composite, similar to a bimetallic strip. This mechanism is applied for the ON/OFF flow switch for controlled delivery of the drug via the device.

In an embodiment, the ON/OFF flow switch comprises a piezoelectric atomizer. The piezoelectric atomizer utilizes the piezoelectric effect to generate high-frequency vibrations, transforming drug suspension into fine mist or droplets without the need for heat or compressed air. The piezoelectric automizer based flow switch comprises a piezoelectric element that is core of the atomizer. The piezoelectric element is a thin piezoelectric plate sandwiched between electrodes. The piezo atomizer operates by applying an alternating electrical current to a piezoelectric transducer. This transducer vibrates at ultrasonic frequencies. The vibration is then transferred to the drug suspension causing it to break up into a mist of tiny droplets. The size of these droplets can be controlled by adjusting the hole sizes on the mesh and varying the frequency and amplitude of the vibrations. Applying an electric field causes the element to vibrate rapidly, typically at ultrasonic frequencies (20 kHz to 10 MHz). When the switch is in on state a predefined dose of the drug flows from the second chamber to a liquid reservoir of the ON/OFF switch. A microfluidic channel connects the reservoir to the nozzle, guiding the liquid towards the vibrating element. The automized drug suspension gets ejected via nozzle to the third chamber, with the aid of the piezoelectric element's vibrations

In some embodiments, the ON/OFF flow switch comprises an actuator comprising electro-thermal actuators and piezoelectric actuators. In some embodiments, the actuators are metal-piezoelectric bilayer membrane actuators. In some embodiments, the plurality of actuators and the second layer comprising the plurality of fluid ports can be formed at one time by micromechanical machining processes. In some embodiments, the microvalve device comprises a fluid source port, a control port and a backflow port, the fluid source port communicates with a fluid source, and the control port is communicated with a mechanism for control of a main valve. In the embodiment, upon the fluid source port and the control port being open and the backflow port being closed, fluid flows from the fluid source port toward the control port; and upon the fluid source port being closed and the control port and the backflow port being open, fluid flows from the control port toward the backflow port.

In one embodiment, a piezoelectric element is integrated into the flow switch, serving as a plug for a microchannel opening. In its natural state (no electric field applied), the piezoelectric element extends, completely occluding the flow path. Upon application of an electric field, the element contracts, creating a gap sufficient for drug passage.

In another embodiment, plurality of piezoelectric elements in sheet form are employed to cover the opening. Precise application of an electric field to the plurality of piezoelectric elements generates a gap between them, enabling drug flow. In specific configurations, the plurality of piezoelectric elements can be arranged to form a diaphragm with a central aperture. Without an electric field, the diaphragm remains closed; however, upon field application, the plurality of piezoelectric elements moves apart, opening the aperture.

In yet another embodiment, piezoelectric elements are positioned at opposing ends of the microchannel opening, converging in the middle to effectively plug the flow path.

FIG. 19S shows ON/OFF mechanism for a flow switch comprising a piezoelectric stack and FIG. 19T shows ON/OFF mechanism for a flow switch comprising a piezoelectric bender.

### Implantable Device Comprising a Shape Memory Alloy Actuator-Based Flow Switch

In an embodiment, implantable device comprising a shape memory alloy actuator-based flow switch is provided. The flow switch comprises a microfabricated unit that uses shape memory polymer (SMP) microtubing, and heating mechanisms. These miniature actuators are of particular interest for use within a small diameter passageway, such as blood vessels or arteries having diameters of about 500-1500 microns. Shape memory polymers distributed by Memory Corporation, can be formed into various configurations and sizes, and thus can be manufactured as small diameter microtubing capable of operating in a 500-1500 micron diameter arteries, blood vessel, or other passageway.

SMP is a polyurethane-based material that undergoes a phase transformation at a manufactured temperature, T_{g}. After the material is polymerized (cross-linked), the material is molded into its memory shape. At a temperature above the T_{g}, the material is soft and can easily be arbitrarily reshaped by applying pressure into another configuration. The elastic constant of the material can change by about 200 times when undergoing this phase transformation. As the temperature is lowered, with the pressure applied to a temperature below the T_{g}, this new shape is fixed and locked in as long as the material stays below the T_{g}. However, if the temperature reheats to above the T_{g}, the material returns to its original memory shape. The SMP material can be heated thermally by heated fluid, resistively, optically, and by external fold (radio frequency (RF) or magnetic induction) heating. The heating is carried out by a laser light source via optical fibers, and with enhanced light absorption due to a coating on the SMP microtubing, an appropriate dye, or a doped polymer.

By inserting into an SMP microtubing, having a specified manufacture temperature, T_{g}, an end of an embolic platinum coil, for example, heating the microtubing to a temperature above the T_{g}, applying pressure to the microtubing causing it to conform to the configuration of the end of the coil, and then cooling to a temperature below the T_{g}, the end of the coil is retained or loaded in the SMP microtubing. By an inverted configuration the end of the SMP microtubing can be inserted into a hollow embolic coil and then heated. The SMP microtubing is then attached to the end of a guide wire or other guidance means and the platinum coil is loaded outside the body of a patient. The guide wire and the loaded coil are then pushed through a catheter in a blood vessel of the body, and at a desired point of use, a brain aneurysm or affected area, for example, the SMP microtubing is heated to a temperature above the T_{g} thereof, such as by injecting warm water through the catheter, resistive heating, external field heating, or optically heating, whereby the SMP microtubing returns to its original memory shape and the end of the coil is released at the desired point of use, whereafter the guide wire and attached SMP microtubing is removed via the catheter. The microtubing can be then cleaned for reuse or disposed of.

The microfabricated SMP release mechanism disclosed above can improve the speed of release of the coil to seconds, compared to the previous 5-30 minutes with the currently used Guglielmi Detachable Coil, and is much more reliable with no known safety hazards to the patient. The release mechanism can also be used in other medical applications requiring the controlled deposition of therapeutic materials, as well as in various non-medical applications. The SMP tubing can be manufactured in various sizes and with different T_{g} temperatures, and thus its use as a release mechanism greatly expands the field of micro-devices for numerous applications.

The following description, with reference to FIGS. 19U-19Y, sets forth an example of the SMP microtubular release mechanism, and loading/release sequence, for use as a release mechanism for therapeutic material, such as an embolic platinum coil or other deposit material. A shape memory polymer (SMP) is manufactured to dimension and shape for an intended target or use, and with a specific phase transformation temperature, T_{g}. FIGS. 19UA-19Y illustrate the loading and release procedure of a straight SMP hollow member or tubing grabbing onto a coil with a ball end. In FIG. 19U the tubing or hollow member 19u10 is in its original size and shape and a coil 19u11 having a ball-end 19u12 can be loaded into the tubing 19u10 as indicated by arrow 19u13. The tubing 19u10 is heated above the T_{g} to soften the SMP material, as indicated at 19u14 in FIG. 19V, and then pressure is applied to the tubing 19v10 in the area of the ball-end 19v12, as indicated at 19v15 in FIG. 19W, whereby the tubing 19w10 is press-fitted over the ball-end 19w12 of coil 19w11. The joined ends of tubing 19w10 and coil 19w11 are then subjected to cooling to temperature below T_{g}, as indicated at 19w16 in FIG. 19X, which stiffens or hardens the SMP material and creates a solid hold of the ball-end 19x12 of coil 19x11 by the end of the SMP tubing 19x10. To release the coil 19x11 from SMP tubing, the joined area of the tubing 19x10 is simply reheated as indicated at 19x17 to above the T_{g}, the tubing 19x10 expands to its original opening and the coil 19x11 is released as indicated by arrow 19x18, as shown in FIG. 19Y. The reheating of tubing 19y10 as indicated in FIG. 19Y can be carried out by injecting warm water, for example, through the tubing. The tubing 19y10 can also be heated and/or reheated by resistive heating, optical heating, or thermal heating.

The amount of heating, pressure, cooling, and reheating is dependent of the diameter and T_{g} of the SMP tubing. For example, with an SMP tubing 19y10 having an internal diameter of 250 μm, an external diameter of 350 μm, and T_{g} of 45° C., with the coil 19y11 having a diameter of 200 μm with a ball-end 19y12 diameter of 250 μm, the SMP tubing is initially heated to a temperature of 48° C., and a pressure of 10 psi is applied to the tubing while maintaining the heat on the tubing to form the press-fit of the tubing around the ball-end of the coil. The SMP tubing is thereafter cooled to a temperature of 37° C., while maintaining the applied pressure, whereby the ball-end of the coil is fixedly retained in the SMP tubing. The coil is released from the SMP tubing by injecting water at a temperature of 48° C. through a surrounding tubing or catheter which causes the tubing temperature to raise above the T_{g} thereof. The SMP tubing can be fabricated with internal diameter of 100 μm to 1000 μm, an external diameter of 150 μm to 1 mm, and with a T_{g} in the range of −30° C. to 100° C.

In some embodiments, when the piston moves towards the egress gate, which is composed of a SMA. The piston has a rod attached to it that pushes into the flexible egress gate, causing it to deform and allowing fluid to exit the tube. When heat is applied directly to the flexible egress gate, it reverts to its original shape, closing the egress site and pushing back against the rod.

In an embodiment, a flexible component made of shape memory alloy (SMA), likely nitinol, forms the valve sealing the drug reservoir. The SMA flow switch acts as a thermally controlled valve, opening when deformed by the piston rod and closing when heated to its original shape. The piston generates continuous force, pushing drug solution towards the egress gate. Attached to the piston is a rod structure, it directly contacts and deforms the SMA gate when the piston advances. A miniature resistive heater or biocompatible heat transfer mechanism is positioned near the egress gate for controlled heating. A control unit regulates heat application to the SMA gate, determining valve opening and closing.

Referring to FIG. 19Z, it shows ON/OFF mechanism for a flow switch comprising an SMA actuator. Initially, the SMA egress gate 19z12b in the flow switch is in its “OFF” configuration, blocking fluid flow. The control unit switches on the flow switch by activating a heat source, warming the SMA egress gate 19z12b. The SMA egress gate 19z12b deforms, bringing the flow switch in ON state and allowing fluid flow. The osmotic pressure driven piston 19z04b advances, pushing drug solution through the open egress gate 19z04b. In some embodiments, the piston 19z04b comprises a piston rod 19z12a comprising a stick slip piezo operable to aid in maintaining the SMA egress gate 19z12b in open configuration. For switching off the flow switch, the control unit stops heating the SMA egress gate 19z12b. The egress gate 19z12b cools and reverts to its original shape, closing the egress site and halting drug flow. The piston rod 19z12a returns to its original position.

### Implantable Device Comprising a Pneumatic Actuation-Based Flow Switch

In an embodiment, a flow switch comprising a miniature pneumatic microvalve is provided. The miniature pneumatic microvalve is controlled by a piezoelectric actuator. The actuator expands or contracts when voltage is applied, manipulating a membrane that opens or closes the flow path between the inlet and outlet channels.

The miniature pneumatic microvalve can be fabricated from biocompatible materials. As described herein, the microvalve features a flexible membrane separating the channels and small ports for controlled pneumatic pressure application. A tiny piezoelectric element integrated within the microvalve structure can convert electrical signals into mechanical movement, influencing the membrane position.

Initially, the piezoelectric actuator remains in its relaxed state, and the membrane in the microvalve blocks the flow path. Similar to other designs, the control unit processes sensor data and commands to deliver specific voltage pulses to the actuator, regulating the membrane deflection and flow state. The control unit sends voltage pulses to the actuator, causing it to expand and push the membrane, opening the flow path and stops sending voltage pulses, allowing the actuator to return to its initial state, causing the membrane to block the flow and complete the cycle.

In some embodiments, the miniaturized pneumatic actuator is hydraulically controlled to regulate flow. The pneumatic pressure opens or closes a small hydraulic valve, controlling the flow of hydraulic fluid that ultimately dictates the drug flow path.

Like the previous embodiment, a tiny pneumatic actuator expands, or contracts based on applied pressure, manipulating the miniature valve for the hydraulic fluid. A hydraulic valve controls the flow of hydraulic fluid, which in turn actuates the main flow control mechanism (e.g., diaphragm). A sealed circuit comprising biocompatible hydraulic fluid connects the hydraulic valve to the main flow control mechanism and allows pressure transfer. A diaphragm directly opens or closes the drug flow path based on the hydraulic pressure received.

At t₀, the micro pneumatic actuator remains relaxed, and the hydraulic valve blocks the fluid flow, keeping the main flow control mechanism closed (drug flow off).

Upon receiving an ON command, the control unit applies pressure to the actuator based on a command received from the microprocessor, the micro pneumatic actuator expands and opens the hydraulic valve. Due to the opening of hydraulic valve, the hydraulic fluid flows through the opened valve, activating the main flow control mechanism to open the drug flow path.

When the control unit receives an OFF command, it releases the pressure, causing the micro pneumatic actuator to relax and the hydraulic valve to close. This stops the hydraulic flow and closes the main flow control mechanism, halting drug delivery.

### Implantable Device Comprising an Electrostatic Actuation-Based Flow Switch

In an embodiment, a flow switch based on electrostatic actuation is provided. The flow switch comprises two micro-machined electrodes (stationery and movable) with a small gap between them. Applying a voltage difference creates an electrostatic force that attracts or repels the movable electrode depending on the polarity. In the “off” state, the movable electrode remains in its default position, blocking the flow path. Applying a specific voltage overcomes the spring force holding the electrode, causing it to move and open the flow channel. Reverse voltage or removing the voltage allows the spring to return the electrode and close the channel. Additional sensors, like pressure or flow sensors, can be integrated to trigger the switch based on specific flow conditions or pressures, offering precise control over drug delivery.

The electrostatic actuator comprises a pair of micro-machined electrodes, an actuation spring, and a control unit. The pair of micro-machined electrodes is fabricated from biocompatible materials like silicon or Parylene. These electrodes are crucial for generating the electrostatic force and defining the flow channel. The actuation spring provides restoring force, ensuring the movable electrode returns to its default position when voltage is removed. The control unit processes input from sensors and external commands to apply the appropriate voltage for switching. The flow switch can be encapsulated in a biocompatible package to ensure safety and sterility within the human body.

Initially, the flow switch remains in the “off” state with the flow channel blocked. Based on a command received by a microprocessor in the device, the control unit applies the required voltage, causing the movable electrode to deflect and open the flow channel. Drug suspension can flow through the opened channel at the desired rate until the pre-programmed conditions are met. After the pre-programmed conditions are met, the voltage can be either automatically removed based on sensor feedback or through an OFF command received by the microprocessor; the spring returns the electrode to its default position, closing the flow channel and stopping drug delivery.

Referring to FIG. 19AA, it shows ON/OFF mechanism for a flow switch comprising an electrostatic actuator, according to one or more embodiments. The flow switch 19aa00 comprises two flexible plate capacitors 19aa06 connected to electrodes 19aa02 within the microchannel. When the flexible plate capacitors 19aa06 are given negative charge, they repel each other, flexing away from each other and opening the microchannel.

Referring to FIG. 19AB, it shows ON/OFF mechanism for a flow switch comprising an electrostatic actuator, according to one or more embodiments. The flow switch 19ab00 comprises two flexible arms comprising electrodes 19ab02 that are connected to plate capacitors 19ab04. The plate capacitors 19ab04 are given negative charge by the control unit, causing them to repel and flex the arms away from each other, opening the microchannel of the flow switch. When the charges are removed, the flexible arms return back to their original state thus blocking the microchannel again.

### Implantable Device Comprising a Rotatory Actuator-Based Flow Switch

In an embodiment, a flow switch based on rotatory actuation is provided. The flow switch incorporates a small, biocompatible rotor positioned within a housing comprising inlet and outlet flow channels. Applying an electric current to the actuator generates torque that rotates the rotor. The rotor's specific geometry acts as a valve, selectively aligning the inlet and outlet channels to allow or block flow. At specific rotation angles, the channels are either perfectly aligned for unrestricted flow (open state) or completely misaligned, blocking the passage (closed state). By controlling the rotation angle and duration, the switch can regulate the volume and duration of drug delivery with high precision.

In an embodiment, the flow switch comprises a rotor, an actuator, a control unit, and a housing. The rotor can be fabricated from biocompatible materials like medical-grade plastics or titanium, the rotor's design defines the flow path configurations at different rotation angles. A compact rotatory actuator, like a micromotor or stepper motor, generates the torque needed to rotate the rotor. Similar to the electrostatic switch, the control unit processes sensor data and external commands to determine the desired rotation angle and duration for precise flow control. The housing encloses the entire mechanism, ensuring sterility and biocompatibility within the implant.

Initially, the rotor rests at a specific angle, blocking the flow path between the inlet and outlet channels. Similar to the electrostatic switch, pre-programmed volume or pressure condition, and/or an external command triggers the drug delivery. The control unit sends signals to the actuator, initiating rotation of the rotor to the designated angle. As the rotor turns, its geometry aligns the inlet and outlet channels, enabling drug solution to flow through at the desired rate. The drug delivery continues until the pre-programmed volume or rotor inlet and outlet channel alignment time duration is reached, or upon deactivation of an external command signal. The control unit then instructs the actuator to reverse the rotation, bringing the rotor back to its initial position and blocking the flow channel.

Referring to FIG. 19AC, it shows an example of a flow switch comprising a rotatory actuation mechanism. The flow path within the microchannel is initially obstructed by a stopper disk. A central axle, aligned with the vertical diameter of the stopper, connects it to an overhead motor. Upon motor activation, the axle rotates, thereby pivoting the stopper disk and opening the microchannel. The cross-sectional outflow area reaches its maximum at a 90-degree rotation angle of the stopper disk.

FIG. 19AD illustrates an example of a flow switch utilizing a gear-driven teethed gate mechanism. A portion of the flow switch housing, extending downward below the microchannel's diameter, serves as a chamber encompassing the lower section of the teethed gate. This exposed gate region meshes with a gear connected to a motor via an axle. When the motor activates, it rotates the gear in a clockwise direction, consequentially pulling the teethed gate downwards. This downward displacement of the teethed gate opens the microchannel's flow path.

FIG. 19AE depicts an example of a flow switch employing a screw-driven actuation mechanism housed within a housing chamber. This chamber comprises a motor, an axle, a threaded cylinder, and a corresponding threaded hole. The motor is axially mobile within the chamber and connects to the threaded cylinder via the axle. Situated atop the threaded cylinder is a flow stop gate. Activating the motor rotates the threaded cylinder, causing its threaded engagement with the hole to translate the motor and cylinder vertically along the axis.

### Implantable Device Comprising a Thermopneumatic Actuation-Based Flow Switch

In an embodiment, a flow switch based on thermopneumatic actuation is provided. The flow switch comprises a small, biocompatible chamber comprising a thermally sensitive element (e.g., shape-memory alloy, polymer gel). Applying a current to heat the element, causing it to expand and deform. The element's expansion pushes against a diaphragm or membrane separating the inlet and outlet flow channels. As the diaphragm deflects, it opens or closes the flow path depending on the desired state. By controlling the heating current intensity and duration, the degree of element expansion and diaphragm deflection can be precisely regulated, enabling accurate control of drug flow volume and duration.

Referring to FIG. 19AF, it shows an example of a flow switch comprising a thermopneumatic actuation mechanism. The flow switch comprises a spring and a microchannel. Part of the microchannel length extends into a chamber below the diameter of the flow switch. This portion of the length has teeth. It is in contact with a gear. On the other side of the gear is a block with the side facing the gear with teeth. This block rests on a flexible chamber comprising a low boiling point liquid. When heat is applied from the wall below the chamber, the liquid expands, flexing the chamber upwards and pushing the left block up. The upwards displacement of the left block turns the gear clockwise, causing downwards displacement of the flow switch block, thereby opening the microchannel for drug suspension flow.

### C. System Comprising Drug Delivery Device and Piston Position Determination Module

An embodiment relates a system comprising an implantable device comprising: a casing that is substantially tubular and has at least a first end, a second end opposite to the first end, a semi-permeable membrane plug at or near the first end, a first chamber, wherein one wall of the first chamber comprises the semi-permeable plug, a second chamber comprising a drug, a piston separating the first chamber and the second chamber, a third chamber comprising a flow switch and an opening for release of the drug from the implantable device into a body of a human or an animal, a fourth chamber near the second end that comprises electronics, and a piston position determination module.

In an embodiment, the piston position determination module can be based on one of a pressure measurement, conductance measurement, resistance measurement, pressure measurement, reflection measurement, capacitance measurement, impedance measurement, radical measurement, image-based measurement, laser measurement, SONAR based measurement, ultrasound measurement, time of flight measurement, and the like, and combination thereof.

Below are described various embodiments related to the piston position determination module.

### Resistance-Based Piston Position Determination

In an embodiment, the piston displacement and therefore the volume of drug dispensed from the device can be determined using resistance measurement. The piston position determination module utilizes principles of electrical resistance, voltage division, and digital signal processing to achieve a contactless and potentially wear-resistant method for tracking linear movements. The core principle relies on the change in electrical resistance as the conductive edge on the piston disk touches different resistive and non-resistive stripes. Resistive stripes function as individual resistors connected in series or parallel, depending on the specific configuration. When the conductive edge touches a non-resistive stripe, the overall resistance remains unchanged. When it touches a resistive stripe, the overall resistance increases proportionally to the stripe's resistance value. The arrangement of stripes and connections can be modeled as a voltage divider circuit. When the piston is centered on a stripe, the voltage drop across the stripe is half the applied voltage. As the piston moves off-center, the voltage drop across the touched stripe and its adjacent stripes changes, providing information about the piston's position. The microprocessor continuously monitors the voltage changes across the stripes. By analyzing the sequence of voltage changes, the microprocessor can determine the specific stripe being touched and calculate the piston's position based on the pre-programmed stripe locations.

In some embodiments, the piston position determination module comprises more complex algorithms to account for factors like non-uniform stripe resistances or temperature variations. The system needs to be calibrated initially to define the exact mapping between voltage changes and piston positions. This calibration might involve measuring the individual resistances of the stripes and their precise locations within the tube.

Referring to FIG. 19AG, the piston 19ag04b is made of polycarbonate or similar non-conductive material and separates the first chamber 19ag04 comprising an osmotic agent O and the second chamber 19ag06 comprising a drug suspension. The piston 19ag04b is well-sealed to prevent leaks and has a precise fit within the tube 19ag02 to ensure consistent resistance readings. The tube 19ag02 is made of a suitable material that can withstand the operating pressure and environment. A plurality of stripes 19ag06a can be placed lengthwise on the inner surface of the tube 19ag02 in the second chamber 19ag06 of the device. The plurality of stripes 19ag06a are made of electrically resistive and corrosion resistant material. In some embodiments, the ends of the plurality of stripes 19ag06a near the middle of the tube 19ag02 are open while the ends at the other end are connected to the electronics. A disk 19ag06b attached to the piston (in the side of piston facing the third chamber 19ag06), features a conductive edge that contacts the resistive stripes 19ag06a within the tube 19ag02. The material of the disk 19ag06b and the conductive edge should be corrosion-resistant and have low electrical resistance. The edge of the piston 19ag04b touches the plurality of stripes 19ag06a connecting the plurality of stripes 19ag06a at a specific point along the second chamber 19ag06. The position of the piston and displacement of the piston d is determined by resistance of the plurality of stripes 19ag06a. A microprocessor continuously monitors the resistance changes across the stripes based on the contact made by the conductive edge. In some embodiments, the microprocessor is present in the fourth chamber 19ag10. In some embodiments, the on-off flow switch is present in the third chamber 19ag08 comprising the microprocessor (not shown in the diagram).

### Reflection-Based Piston Position Determination

In an embodiment, the piston displacement, and therefore the volume of drug dispensed from the device, can be determined using reflection measurement. The piston position determination module utilizes a mechanism that comprises time-of-flight measurement, reflection, light detection, signal processing, and volume calculations to achieve a non-contact and potentially wear-resistant method for tracking piston movement and dispensed volume. The piston position is determined based on the time it takes for light to travel from a light emitting device (LED) to the piston and back to a photodiode. By knowing the speed of light, the microprocessor can calculate the piston's position based on the measured time difference. The piston's reflective surface plays a crucial role in accurately directing the light beam back towards the photodiode. The quality and alignment of the reflective surface directly affect the signal strength and measurement accuracy. The LED and photodiode need to be suitable for the operating environment and the desired volume measurement range. The photodiode converts the reflected light into an electrical signal. The intensity of this signal corresponds to the amount of reflected light, which can be affected by factors like distance, surface reflectivity, and ambient light. The microprocessor analyzes the timing and intensity of the received signal. Based on pre-programmed parameters and calibration data, the microprocessor extracts the distance information from the signal and calculates the piston's position. By correlating the piston's position change with the known chamber volume, the microprocessor can calculate the dispensed volume. In some embodiments, the piston position determination module comprises more complex algorithms to account for factors like signal noise, variations in light intensity, and temperature changes.

If the initial light intensity is Q₀ and the initial length of the drug chamber is L₀, the microprocessor measures the voltage across the photodiode which is proportional to the light intensity. As the piston moves the light intensity and hence the detected voltage, changes proportional to the square of the distance L.

If the change in light intensity is detected Q₁, the length of the drug chamber, after a certain amount of drug has been dispensed, L₁, can be calculated by:

$$\frac{{Q}_{1}}{{Q}_{0}} = {( {L}_{1} \vert {L}_{0} )}^{2} \\ {L}_{1} = {L}_{0} * \sqrt{\frac{{Q}_{0}}{{Q}_{1}}} \qquad Equation 2$$

Referring to FIG. 19AH, piston 19af04b comprises a reflective surface 19ah06b (mirror or highly reflective material) to accurately reflect light beams. A light emitted diode (LED) 19ah06c is positioned within the tube 19ah02, that is operable to generate a focused light beam through the drug chamber 19ah06 (line with arrow) towards the reflective surface 19ah06b. A flow switch 19ah16, mounted in the fluid path from second chamber to the third chamber for drug suspension D, detects the passage of fluid and triggers the piston position determination module for volume calculations. A photodiode 19ah06a mounted opposite the LED 19ah06c, detects the reflected light beam from the reflective surface 19ah06b. In some embodiments, the LED 19ah06c and the photodiode 19ah06a are attached to the flow switch 19ah16 and are connected to the fourth chamber 19ah10 via the flow switch 19ah16. The fourth chamber 19ah10 comprises the microprocessor and control circuitry (not shown in the diagram).

The LED 19ah06a emits a focused light pulse towards the piston's reflective surface 19ah06b. The reflective surface bounces the light back towards the photodiode 19ah06c. The photodiode 19ah06c converts the reflected light into an electrical signal. The microprocessor analyzes the signal's timing and intensity. The time it takes for the reflected light to reach the photodiode is proportional to the distance between the LED and the piston (light travels at a constant speed). By knowing the initial distance between the LED and the piston at its starting position (which can be changed via measurement intervals) or the default position (always measuring piston position from default (consistent) position), the microprocessor can calculate the current piston position based on the time difference. When the flow switch detects fluid passage, the microprocessor correlates the piston's position change with the known chamber volume to calculate the dispensed volume.

### Conductance-Based Piston Position Determination

In an embodiment, the piston displacement and therefore the volume of drug dispensed from the device can be determined using conductance measurement. The inside wall of the implant's drug chamber is covered by a pattern of equally spaced metal rings. These rings are connected to the CPU via individual lines. The piston has a metallic edge that makes a short across a ring as it moves from one end to the other end of the drug chamber. The microprocessor detects which ring is shorted by the piston, thereby determining how much drug has been dispensed. The number of metal rings and their spacing is determined by the number and volume of dosage.

In an embodiment, the second chamber comprising the drug suspension D, further comprises a plurality of metal rings M equally spaced along its inner wall. The plurality of metal rings is arranged in a precise pattern along the second chamber's length, each connected to an individual line that leads to the fourth chamber comprising a microprocessor and a power supply. The piston comprises a cylindrical structure with a metallic edge, capable of sliding smoothly within the second chamber. As the piston moves along the chamber, its metallic edge progressively contacts and short-circuits the corresponding metal ring(s) in sequence. The plurality of metal ring, as they cause a short, causes a change in electrical conductance, detected by a microprocessor. The microprocessor is responsible for monitoring the electrical signals from the metal rings and calculating the piston's position. When a ring is shorted, the conductance of its corresponding line drops significantly. By identifying which ring is currently shorted, the microprocessor can precisely determine the piston's position within the chamber. The number of rings and their spacing are carefully calibrated to correlate with the drug chamber's volume and the desired dosage amounts. This allows the CPU to accurately calculate the dispensed drug volume based on the piston's position.

Referring to FIG. 19AI, the inner circumference of the second chamber 19ai06 comprising the drug suspension D is covered by a pattern of equally spaced rings. The plurality of rings are connected to the microprocessor via plurality of individual lines L. The piston 19ai04b has a metallic edge E that makes a short across the plurality of rings as the piston 19ai04b moves from one end to the other end of the second chamber 19ai06. The microprocessor detects which of the plurality of rings is shorted by the piston 19ai04b, thereby determining how much drug suspension has been dispensed via flow switch 19ai16. The number of the plurality of rings and their spacing is determined by the number and volume of dosage of D.

### Salt Solution Conductance-Based Piston Position Determination

In an embodiment, the osmotic pressure in the salt chamber can be used to measure the piston position. The conductance of a salt solution demonstrates a measurable trend as the salt concentration decreases within a confined volume. Conductance, representing the solution's ability to conduct electrical current, is intimately tied to the concentration of dissolved ions, specifically sodium and chloride in the case of a salt solution. With decreasing salt concentration, the number of ions available to facilitate electrical conductivity diminishes, resulting in a proportional reduction in conductance. As the salt concentration decreases, the solution's capacity to transmit an electric charge decrease. This relationship between conductance and salt concentration can be used to measure the piston position inside the tube.

In an embodiment, the salt concentration can be used to measure the piston position. Conductivity (G) is directly proportional to salt concentration C_{w} (example of salt: NaCl), which is inversely proportional to the volume (V) of the osmotic chamber. V is directly proportional to the length of the osmotic chamber (l). The displacement of the piston is d, the relationship between d and C is:
wherein

$$d = ( {h}_{2} - {h}_{1} ) = \frac{a}{( {G}_{2} - b )} - \frac{a}{( {G}_{1} - b )} \qquad Equation 3$$

a is a constant dependent on the radius of the cylinder and amount of a solute (salt); and
b is a constant, the offset in the relation between G and Cw.

FIG. 19AJ shows the relationship between Conductivity G and salt concentration C. The salt solution is not to be electrolyzed during measurement of conductance and the device measures the conductance in the linear zone of the G to C curve.

FIG. 19AK shows a device comprising electrodes for measurement of solution conductance. FIG. 19AK also shows a circuit diagram to measure conductance of the salt solution O using a current meter. The device comprises two electrodes 19ak10e in the form of an inert conductive wire (for example, gold wires) that are placed diametrically opposite each other on the inner wall of the tube 19ak02. The electrodes, wires, 19ak10e connect from the first end of the device, comprising the semi-permeable membrane 19ak04a, past the piston 19ak04b, thru the drug delivery chamber, past the flow switch which is the third chamber until the fourth chamber 19ak10, where two probes are used to measure the conductance. The two electrodes 19ak10e are in the form of long stripes to get a clean average conductance.

FIG. 19AL shows a circuit diagram to measure conductance of the salt solution, via the device, according to one or more embodiments. Thin gold wires or plates can be used as electrodes 19a110e and are placed diametrically opposite each other on the inner wall of the tube 19a102. The electrodes connect the first end comprising the semi-permeable membrane 19a104a, past the piston to the fourth chamber 19a110 where two probes are used to measure the conductance. In some embodiments, a simple microprocessor control circuit and/or Analog to Digital Converter ADC is used to measure the changes in the current. The circuit is to be kept open and connected only when the conductivity is to be measured (during drug release), else it will drain the battery 19a110b. This can be achieved by the electronic switch operated by a microprocessor. Further, a Wheatstone bridge is used to increase sensitivity.

### Osmotic Pressure-Based Piston Position Determination

In an embodiment, the piston position is inversely proportional to the osmotic pressure exerted by the salt solution. If piston is at a position h1 and time t1 and reached to h2 at time t2, and the salt solution is NaCl, the displacement d can be calculated using the formula:

$$d = ( {h}_{2} - {h}_{1} ) = \frac{2 MRT}{A} ( \frac{1}{{π}_{2}} - \frac{1}{{π}_{1}} ) \qquad Equation 4$$

Wherein:
M is molarity of the solution,
T is the temperature,
A is cross sectional area of drug suspension chamber, and
π₁ and π₂ are osmotic pressure exerted by the salt solution at time t₁ before dispensing of drug and at time t₂ when the drug is dispensed through the flow switch.

Referring to FIG. 19AM, it shows a drug delivery device comprising an osmotic pressure-based piston displacement monitoring module (module not shown), according to one or more embodiments. The device comprises an osmotic pressure sensing unit in the first chamber 1304, which comprises the osmotic agent O. In some embodiments, the osmotic pressure sensing unit is positioned adjacent to the semi-permeable membrane 1304a. The osmotic pressure sensing unit comprises a pressure sensor 1316, a second electronic unit 1316a, and a disc holder with a flow channel 1316b for the interstitial fluid to flow into the first chamber 1304 through the semi-permeable membrane 1304a.

Upon the release of drug from the third chamber 1308 into the subject's body, triggered by the activation of the flow 1310, drug dispensing occurs, causing the piston 1304b to move towards the second chamber 1306. As the piston 1304b moves toward the second chamber 1306 comprising the drug D, more interstitial fluid diffuses through the semi-permeable membrane 1304a into the tube, resulting in a decrease in salt concentration and, consequently, a decrease in osmotic pressure.

The osmotic pressure sensor 1316 records this pressure change and can be used to calculate the displacement of the piston 1304b, determining the volume of drug dispensed from the second chamber 1306 via the flow switch 1312 by a microprocessor. The pressure sensor is configured to measure pressure only after one ON/OFF switch cycle when the osmotic pressure is at equilibrium in the off state.

Referring to FIG. 19AN, it displays an exploded view of the end cap of the device tube, which comprises a semi-permeable membrane and an osmotic pressure-based piston displacement monitoring module. The end cap 19an04a comprises a pressure sensor 19an16 that is affixed to a holder comprising electronics 19an16b, and this assembly is connected to a rigid disc 19an16a comprising flow channels 19an16c for the smooth passage of interstitial fluid entering the tube 19an02 adjacent to the semi-permeable membrane 19an04a. The semi-permeable membrane 19an04a is situated between two spacer discs 19an04i and 19an04j, tightly held in place to facilitate the diffusion of interstitial fluid. The spacer discs 19an04i and 19an04j also functions as a chamber for the diffusion of interstitial fluid through the semi-permeable membrane 19an04.

Referring to FIG. 19AO, it shows a drug delivery device comprising an osmotic pressure-based piston displacement monitoring module juxtaposed/attached to an ON/OFF flow switch, according to one or more embodiments.

In some embodiments, the device is operable for metered drug dosing by: (1) measuring displacement of the piston using conductivity and pressure sensors located in the osmotic chamber, and (2) discharging the drug using an on-off flow switch controlled by a microprocessor based on the desired displacement of the piston.

### Displacement of the Piston as a Function of Conductivity and Osmotic Pressure

The osmotic pressure in the osmotic pump is as follows: π=icRT where π is the osmotic pressure and i is the Van′t Hoff factor and c is the concentration of NaCl.

$$c = \frac{π}{iRT} \frac{1}{c} = \frac{iRT}{π}$$

Assume L₁ is the initial and L₂ the final position of the piston before and after displacement of the piston under osmotic pressure.

The piston displacement as a function of osmotic
pressure π is calculated as follows:
$\begin{matrix}{L}_{2} - {L}_{1} = \frac{M}{A} ( \frac{1}{{C}_{2}} - \frac{1}{{C}_{1}} ) \\ {L}_{2} - {L}_{1} = \frac{MiRT}{A} ( \frac{1}{{π}_{2}} - \frac{1}{{π}_{1}} )\end{matrix}$
i = 2 for NaCl
Piston displacement D = L₂ − L₁
$D = \frac{2 MRT}{A} ( \frac{1}{{π}_{2}} - \frac{1}{{π}_{1}} ) \qquad Equation 5$
The piston displacement as a function of electrical
conductivity σ is calculated as follows:
$\begin{matrix}{V}_{1} = {L}_{1} {Ac}_{1} = \frac{M}{{V}_{1}} \\ {c}_{1} = \frac{M}{{L}_{1} A} {c}_{2} = \frac{M}{{L}_{2} A}\end{matrix}$
${L}_{1} = \frac{M}{{c}_{1} A} {L}_{2} = \frac{M}{{c}_{2} A} and therefore {L}_{2} - {L}_{1} =$
$\frac{M}{A} ( \frac{1}{{c}_{2}} - \frac{1}{{c}_{1}} ) \frac{1}{c} = 5.787 \frac{1}{σ}$
$D = 5.787 \frac{M}{A} ( \frac{1}{{σ}_{2}} - \frac{1}{{σ}_{1}} ) \qquad Equation 6$

As per the above equations, displacement of the piston is determined by measuring electrical conductivity and osmotic pressure in the osmotic pump. To start a dose, the on-off flow switch is turned on. Then, when the piston is displaced by a desired distance D, the on-off flow switch is turned off. As such, the device discharges a precise dose from the drug chamber to the exterior of the device into a human body when the device is implanted in the human body.

In an embodiment, the pressure sensor is an absolute digital output barometer with water-resistant package. The pressure sensor is an ultra-compact piezoresistive absolute pressure sensor designed for digital output barometer applications. It features dual full-scale absolute pressure modes (260˜1260 hPa in Mode 1 and 260˜4060 hPa in Mode 2), with low power consumption down to 1.7 μA. The sensor provides high accuracy (0.5 hPa), low pressure noise (0.32 Pa), and includes embedded temperature compensation for accurate readings over a temperature range of −40° C. to +85° C. It communicates via I²C or MIPI I3CSM interfaces, offers a 24-bit pressure data output, and supports ODR from 1 Hz to 200 Hz. Additionally, it includes features like embedded FIFO, interrupt functions (data-ready, FIFO flags, pressure thresholds), and operates on a supply voltage range of 1.7 to 3.6 V. The sensor is housed in a water-resistant ceramic LGA package with a metal lid, optionally connected to ground or left floating depending on the application and features an easily sealed package with an O-ring for protection.

In some embodiments, a conductivity sensor is used to measure conductance of the osmotic agent in the osmotic chamber of the device. The concentration of osmotic agent will vary from 0 millimolar to 200 millimolar. This solution represents a wide range of salinity levels, and the sensor must accurately measure conductivity under these conditions.

In some embodiments, the conductivity sensor has a conductivity range from 0 μS/cm (low conductivity) to 200 mS/cm (high conductivity). Therefore, it can accurately measure the electrical conductivity of the surrounding medium changing pressure inside the osmotic chamber within this specified range. In some embodiments, the conductivity sensor functions reliably within the temperature range of 25° C. to 40° C. This ensures its stability and accuracy across varying environmental conditions.

In some embodiments, the conductivity sensor comprises an integrated Resistance Temperature Detector (RTD) sensor (For example Pt1000) for precise temperature compensation. The Pt1000 sensor has a base resistance value of 1000 Ohms at 0° C. It is suitable for 2-wire circuit configurations, has less significant impact of lead wire resistance, consumes less power due to higher resistance, and is typically available only with thin-film element constructions.

In some embodiments, the conductivity sensor is designed to fit snugly inside a cylindrical space with specific dimensions (for example within 5 mm*4 mm). This compact design allows for easy integration into various applications. In some embodiments, the pressure sensor's housing material is titanium, which provides durability, corrosion resistance, and compatibility with the fluid it will be immersed in.

In some embodiments, the conductivity sensor is configured to prevent electrolysis. This means it won't undergo chemical reactions that could alter its performance or degrade its materials. Pt (platinum) based electrodes are used to prevent hydrolysis. These electrodes ensure stable performance even in the presence of water.

In some embodiments, the conductivity sensor uses 0.3-1 VP—P (peak-to-peak voltage) for operation. In some embodiments, the pressure sensor 0.7 VP—P (peak-to-peak voltage) for optimal operation.

In some embodiments, the conductivity sensor allows for simulating the cell constant, which relates to its geometry and electrical properties.

A simulation and experiment were conducted with the conductivity sensor using different cylinder dimensions: Cylinder 1 having dimension 5 mm×15 mm and Cylinder 2 of dimension 10 mm×20 mm. Referring to FIG. 19AP and FIG. 19AQ, conductivity of the medium a was 14 mS/cm at frequency, f=1000 Hz and the calculated cell constant was 0.317 cm⁻¹.

### Touch Membrane Piston Position Determination Module

In one embodiment, a touch membrane is used in the device for measuring the piston displacement in the tube, and therefore the amount of drug present in the device. The touch membrane is a resistive touch membrane that is used in the electrostatic capacity type. In another embodiment, the touch membrane is a conductive film is used in the electrostatic valve application. In yet another embodiment, the touch membrane is a hybrid film comprising a resistive film and a conductive film capable of detecting both touch, capacitance and pressure exerted due to piston displacement. In some embodiments, the touch membrane comprises a piezo sensor that can be used to sense piston position. In an embodiment, a piezo resistance sensor can be used to sense piston position. In some embodiments, the piston comprises a metal wire. In some embodiments, piezo resistance sensor is a flat piezo resistance sensor. In some embodiments, piezo resistance sensor is a flexible piezo resistance sensor rolled up inside the body of the device casing in the first and second chambers.

Referring to FIG. 19AR, the device features a resistive touch membrane 19ar10a lining the inner surface of the tube 19ar02 in the second chamber 19ar06. The resistive touch membrane 19ar10a comprises plurality of conducting surfaces (for example, C₁ and C₂) and a soft, non-conducting layer (I). The plurality of conducting surfaces (C₁ and C₂) form the top and bottom layers of the resistive touch membrane, acting as electrodes. The soft, non-conducting layer (I) is sandwiched between the conducting layers. The soft, non-conducting layer (I) acts as a flexible material to enhance sensitivity and ensure smooth piston 19ar04b contact. The resistive touch membrane functions as a narrow, flexible strip running along the second chamber's length. At the initial stage, the piston 19ar04b rests between the first chamber 19ar04 and second chamber 19ar06, not touching the resistive touch membrane 19ar10a. This guarantees a uniform electrical resistance across the entire strip. As the piston 19ar04b advances towards the ON/OFF flow switch 19ar12, it gently presses against the resistive touch membrane 19ar10a, bridging the gap between the two conducting surfaces (C₁ and C₂) at a specific point. This physical contact creates a measurable resistance (Rx), which is continuously monitored by a microprocessor that is placed in the fourth chamber 19ar10 through electrodes (e₁ and e₂).

By analyzing the change in resistance (Rx) and referencing a pre-programmed map of resistance values along the resistive touch membrane strip, the microprocessor can precisely pinpoint the piston's location. Knowing both the initial and current positions, the microprocessor calculates the distance traveled by the piston, directly translating to the dispensed drug volume.

### D. Implantable Device Comprising a Check Valve

In an embodiment, the device comprises a flow switch comprising a check valve. Non limiting examples of check valve are swing check, relief valve, spring loaded check valve, diaphragm check valve, left check valve, stop check valve, dual plate check valve, in line check valve, tilting disc check wall, wafer check valve, and spring check valve. Valves can be actuated using different methods, such as mechanical, pneumatic, electrostatic, electromagnetic, or piezoelectric actuation. In some embodiments, the valve is a miniaturized one-way check valve. The check valve is designed to protect equipment from backflow damage, provide pressure relief for system safety and to prevent contamination from reverse flow.

### Device Comprising Relief Valve

In an embodiment the flow switch comprises a relief valve. The relief valve comprises a ball and electromagnetic actuator. The electromagnetic actuator comprises a permanent magnet and a push rod. A valve body provides the housing for all the internal components. It has an inlet and an outlet through which fluid flows. The ball is a spherical closure element inside the valve. It is positioned in such a way that, in its natural state, it rests against the valve seat to block fluid flow. The ball acts as a flow restrictor for the drug present in the inside of the pressurized drug chamber due to an osmotic pressure exerted by an osmotic chamber. The electromagnetic coil is a wound wire coil connected to a power source. When an electric current passes through this coil, it generates a magnetic field. The rod is a component that extends from the electromagnetic coil to the ball. It is designed to transmit electromagnetic force to the ball. The relief valve is normally closed due to fluid pressure and is designed to block flow through the valve. The electromagnetic actuator is a linear actuator capable of moving and reaching discrete positions based on the amount of current applied. In the off state, the ball blocks a flow channel between the drug reservoir chamber and the drug delivery chamber and prevents the drug from flowing out from the drug reservoir chamber to the drug delivery chamber. In some embodiments, the ball is held by a socket comprising a spring. The ball check valve is designed to use electromagnetic forces to control the position of a ball within the valve, allowing or preventing the flow of fluid. FIG. 19AS and FIG. 19AT show a schematic of an implantable drug delivery device comprising a relief valve in OFF state and ON state, respectively, according to one or more embodiments.

Closed State is the default state of the relief valve. In the closed state, the electromagnetic coil is not energized. As a result, the rod remains in its resting position, and the ball is seated against the valve seat, blocking the flow of fluid. The spring may assist in keeping the ball in the closed position. When the valve needs to be opened, an electric current is applied to the electromagnetic coil. The coil generates a magnetic field, and this magnetic field exerts a force on the rod, pulling it towards the ball. This movement of the rod results in the ball being lifted off the valve seat. With the ball lifted, fluid can now flow through the valve from the inlet to the outlet. The electromagnetic force overcomes the weight of the ball, allowing it to move against the back force of the fluid. When the electric current to the electromagnetic coil is cut off, the magnetic field dissipates. The spring then exerts a force on the rod, causing it to return to its resting position. As the rod moves back, the fluid pressure brings the ball back down to its seated position against the valve seat, closing the valve.

Referring to FIG. 19AU, it shows a schematic of ON/OFF mechanism of flow via relief valve, according to one or more embodiments. In the ON state when a current is applied to the actuator it pushes the rod to displace and pushes the ball towards the drug reservoir chamber allowing fluid or drug to flow out via the flow channel. Releasing the current would allow the ball to close again due to a spring force, blocking any further flow. The amount of current applied could control how far the push rod displaces and the ball opens, regulating the flow rate. This provides a way to precisely control drug delivery by actuating a relief valve.

In an embodiment, the relief valve comprises a push-pull solenoid. The push-pull solenoid valve is a type of solenoid that can either pull or push based on how it is activated. When something is attached to the shaft, it can pull, and when the shaft is rotated the other way around with something in front of it, it can push. These solenoids are convenient because they come with everything needed for operation, including a spring for returning the shaft to its original position and a retainer ring for the spring. This makes them easy to set up and use.

The construction of a push-pull solenoid comprises three primary components a) a coil that is wound around a bobbin, forming the body of the solenoid, b) a plunger positioned within the coil that moves under the influence of the magnetic field and c) a return spring located at the opposite end of the plunger. The return spring pushes the plunger back to its original position when the current is turned off, resetting the solenoid for the next operation. When activated, the solenoid either pushes the drug out of the reservoir or pulls it into a delivery channel. The release rate can be precisely controlled, ensuring consistent drug levels.

In an embodiment, the relief valve comprises a super stroke solenoid. The super stroke solenoid is an electromechanical device designed for controlled fluid flow.

Referring to FIG. 19AV, it shows working of a super stroke solenoid for controlled drug release, according to one or more embodiments. The core 19av02 is a coil of wire (not shown in the diagram) wound around a cylindrical form. When an electric current passes through the coil, it generates a magnetic field. Inside the coil, there's a movable plunger or piston 19av04. This plunger 19av04 is typically made of ferromagnetic material. The plunger is attached to a spring 19av06. When the solenoid is not energized, the spring 19av06 keeps the plunger 19av04 in a retracted position. When an electric current flows through the coil, the magnetic field attracts the plunger 19av04. The plunger 19av04 moves towards the coil, compressing the spring 19av06. As the plunger 19av04 moves, it opens or closes the fluid pathway. When the solenoid is energized, fluid flows through the open pathway. When de-energized, the spring 19av06 pushes the plunger 19av04 back, blocking the flow. In some embodiments, the solenoid has dedicated ports for fluid entry from the drug reservoir chamber and exit to drug delivery chamber.

In some embodiment, the device comprises a stepper motor actuator. The stepper motor actuator is used to actuate the relief valve. FIG. 19AW shows an implantable device comprising a check valve and a stepper motor actuator in an ON state, according to one or more embodiments. The stepper motor actuator converts electrical pulses into controlled mechanical movements. In the ON state the stepper motor directly drives the valve stem or actuator arm, causing the relief valve to open or close. In some embodiments, the stepper motor may drive a gear mechanism or a screw, which, in turn, will operate the relief valve. This indirect method may allow fine adjustments and prevent sudden valve movements. In some embodiments,

In some embodiment, the device comprises a vibration motor actuator for actuating the relief valve. FIG. 19AX shows an implantable device comprising a check valve and a vibration motor actuator in an ON state, according to one or more embodiments. The vibration motor will drive the valve stem or actuator arm, causing the relief valve to open or close.

### Device Comprising Spring Loaded Check Valve

In an embodiment, the flow switch comprises a spring-loaded check valve. The spring-loaded check valve comprises a valve body, a tail structure, an electromagnetic coil, and an armature. The valve body provides the housing for all the internal components and features an inlet and an outlet for fluid flow. The tail structure is a flexible or hinged element attached to the valve body. This tail structure serves as the closure element that blocks the flow when the valve is in its default or closed state. The electromagnetic coil is wound wire connected to a power source. When an electric current passes through this coil, it generates a magnetic field. The armature or attraction plate is a component positioned near the tail structure and is attracted by the magnetic field generated by the electromagnetic coil. In some embodiments the armature comprises a rod structure to connect the tail structure to an attraction plate. In some embodiments, a spring is utilized to provide a restoring force to the tail structure. When the electromagnetic force is deactivated, the spring assists in returning the tail to its original position. FIG. 19AY and FIG. 19AZ show a schematic of an implantable drug delivery device comprising a spring-loaded check valve in OFF state and ON state, according to one or more embodiments.

For the spring-loaded check valve embodiment, the actuation mechanism works as follows: The spring-loaded check valve is placed inside the drug chamber with the ball inside and spring outside, opposite of its normal configuration. With this inverted setup, any pressure built up inside the chamber cannot force the ball to open, blocking drug flow. An electromagnetic actuator has a permanent magnet and a push rod. Applying current to the actuator causes the push rod to displace and pushes the ball inside the spring-loaded check valve up against the spring force. This partial opening of the ball allows a controlled amount of drug to flow out of the chamber through the gap. Releasing the current allows the spring to push the ball back into the closed position, stopping further drug release. The amount of current controls the displacement of the push rod and how open the ball is, regulating the drug flow rate.

FIG. 19BA shows a schematic of ON/OFF mechanism of flow via the spring-loaded check valve. The tail structure, being in its default position, blocks the fluid flow path, preventing the flow of fluid through the valve. When the valve needs to be opened, an electric current is applied to the electromagnetic coil. The coil generates a magnetic field, attracting the armature or attraction plate towards it. The attracted armature or plate exerts force on the tail structure, causing it to move away from its default position. The flexible or hinged design of the tail allows it to swing or pivot away from the flow path. As the tail structure moves, it uncovers the flow path, allowing fluid to pass through the valve from the inlet to the outlet. The electromagnetic force overcomes the spring force and the weight of the tail, allowing it to move against gravity. When the electric current to the electromagnetic coil is cut off, the magnetic field dissipates. The spring then exerts a force on the tail structure, causing it to return to its default position. As the tail moves back, it covers the flow path, closing the valve.

### Device Comprising Spring Type Check Valve

In an embodiment the flow switch comprises a spring type check valve. The spring type check valve comprises a generally tubular body including a cylindrical base and a guide sleeve of reduced diameter which axially extends from the base. The body forms an interior valve seat which surrounds an axially extending fluid passageway. The guide sleeve has a plurality of angularly spaced discharge openings and a pair of pilot orifices which are axially spaced from the valve seat. A valve ball is displaced from the valve seat to form a fluid flow path which extends axially through the valve seat and into the guide sleeve and thereafter radially through the orifices and openings for flow exteriorly of the guide sleeve. The ball is biased by a spring assembly which comprises a pair of axially aligned springs having a relatively low pre-load force and low spring rate. The ball and seat valving configuration, in combination with the tubular ball guide, the discharge window locations and the pilot orifice locations, forces the valve member to open a substantial axial distance at relatively low flow pressures to thereby eliminate or minimize silting or contamination conditions. The valve is also very stable because of the second order damping provided by the multiple spring assembly and damping chambers. The substantially radial flow path presents a relatively unobstructed flow path through the check valve and further enhances the low flow resistance of the check valve.

FIG. 19BB is an axial sectional view of a check valve in accordance with the present invention illustrated in combination with a fluid conduit in which the valve has been mounted; FIG. 19BC is a fragmentary side view of a selected portion of the check valve of FIG. 19BB; and FIG. 19BD is a fragmentary cross-sectional view of the check valve and fluid conduit taken along the line 3-3 of FIG. 19BC. Referring to FIG. 19BB, the miniaturized check valve 19bb10 comprises a tubular insert 19bb20 which comprises a plug 19bb22 of enlarged diameter and an axially extending sleeve 19bb24 which integrally extends from the plug. The exterior downstream end of the plug 19bb22 forms a tapered shoulder 19bb26 which in complementary fashion forcefully seats against the shoulder 19bb16 of the counterbore. The plug is exteriorly dimensioned so that the plug is closely received in the enlarged portion of the counterbore. A plurality of circumferentially extending axially spaced grooves 19bb28 traverse the exterior surface of the plug to form alternating axially spaced sealing lands and grooves. The outside diameter of the sleeve 19bb24 is generally uniform and is less than the diameter of the reduced portion of the conduit bore so that an annular passageway is formed in the reduced bore portion between the sleeve 19bb24 and the wall of the fluid conduit 19bb12.

An enlarged tapered central axially extending bore 19bb30 extends from the upstream end of the plug and communicates with a reduced bore 19bb32 which extends through the opposite end of the plug. The check valve 19bb10 is mounted in the fluid conduit by inserting the insert 19bb20 into counterbore 19bb14 so that the shoulder 19bb26 of the plug axially seats against the counterbore shoulder 19bb16. A tapered pin (not illustrated) is inserted into the tapered bore 19bb30. The pin and the plug are dimensioned so that as the tapered pin is forcefully axially driven into the plug (to the right in FIG. 19BB), the pin forces the plug to radially expand to thereby force the plug to sealingly engage the wall of the fluid conduit 19bb12 in a fashion wherein the edges of the plug bite into the surrounding material of the conduit to form independent seals and retaining rings with the conduit. The expanded tapered bore subsequently functions as a fluid inlet passageway when the check valve is mounted in position as illustrated.

Reduced bore 19bb32 opens into an enlarged uniform bore 19bb34 which axially traverses the length of sleeve 19bb24. The upstream end of bore 19bb34 is defined by an annular shoulder 19bb36 which intersects the end of the wall of bore 19bb32 to form a sharp, well-defined continuous circular edge 19bb38 which functions as a valve seat.

A valve member in the form of a spherical ball 19bb40 is received in bore 19bb34. Ball 19bb40 has a diameter which is only slightly less than the diameter of the bore 19bb34. Ball 19bb40 is axially displaceable for sealing engagement with seat 19bb38 to prevent fluid flow through the check valve. Ball 19bb40 is normally biased to the closed or seated position of FIG. 19BB by a spring assembly designated generally by 19bb42. In the seated position, the ball also intersects the wall of the bore 19bb34 along a circular path axially spaced from seat 19bb38 to form a quasi-secondary valve seat engagement with sleeve 19bb24. Spring assembly 19bb42 includes a follower 19bb44 forming a recess which is contoured to symmetrically engage against ball 19bb40 to urge (to the left in FIG. 19BB) the ball to the closed seated position. The follower 19bb44 has a cylindrical surface with a diameter which is approximately commensurate with the diameter of bore 19bb34. Follower 19bb44 is slidably received in sleeve 19bb24 and axially displaceable therein. A central bore 19bb46 extends axially through the follower to provide a vent passage so that the ball may be firmly engaged against the follower. The downstream end of the follower forms a central axially opening recess 19bb48. The recess 19bb48 defines a retainer for receiving one end of a coil spring 19bb50. The other end of coil spring 19bb50 is captured in a retainer recess of an axially displaceable damping member 19bb52. Damping member 19bb52 contains an opposing, axially spaced retainer recess for capturing an end of a second coil spring 19bb60. The damping member 19bb52 has a generally cylindrical surface which is closely received by sleeve 19bb24 to permit a restricted damped axial movement of the damping member 19bb52. A damping bore 19bb54 extends through the damping member 19bb52 coaxially with the sleeve 19bb24. A generally cup-shaped end cap 19bb62 is received at the downstream end of sleeve 19bb24 and welded in fixed position to the sleeve. End cap 19bb62 forms a retainer recess for seating the opposite downstream end of spring 19bb60. Cap 19bb62 also contains a central damping bore 19bb64 which axially aligns with the corresponding axially spaced damping bore 19bb54. The damping bores 19bb54 and 19bb64 have generally equal diameters and cooperate with bore 19bb34 to form a damping passage through the spring assembly 19bb42. Springs 19bb50 and 19bb60 are relatively short springs which are substantially identical and have a relatively low spring rate and low preload force. An exemplary spring rate is 0.4 lbs. per inch for each spring. Thus, the dual spring configuration has an effective spring rate of 0.2 lbs. per inch. The dual spring configuration adds stability to the check valve while also allowing for sufficient axial displacement of ball 19bb40 as described hereinafter. The damping member 19bb52 functions as a guide member to prevent intermediate spring buckling or deformation that results from employing an equivalent single longer spring having a small pre-load force. The intermediate axially displaceable damping member 19bb52 in cooperation with springs 19bb50 and 19bb60 functions to provide a damping mechanism to thereby minimize or alleviate deleterious oscillations and chatter in the valve mechanism. The reverse flow of fluid created by a reverse pressure differential traverse through the damping bores 19bb64 and 19bb54 facilitates the damping process. Alternately, a series of three or more axially aligned short springs having a relatively low spring rate and low pre-load force with intermediately disposed damping members may be employed rather than the illustrated dual spring assembly.

Four equiangularly spaced, substantially identical discharge windows 19bb70, 19bb72, 19bb74 and 19bb76 are formed in sleeve 19bb24. The foregoing windows have a generally rectangular shape and are axially positioned so that the upstream terminus of the windows is axially spaced downstream from the intersection of the ball 19bb40 and the inner wall of the sleeve 19bb24 when the ball is seated against seat 19bb38. A pair of diametrically opposed notches at an intermediate location of the upstream boundaries of windows 19bb70 and 19bb74 form a pair of opposed pilot orifices 19bb80 and 19bb82. The upstream terminus of the orifices is axially spaced from the intersection of the ball 19bb40 with the sleeve 19bb24 in the seated closed valve position. The pilot orifices are dimensioned to provide both a significantly smaller opening in the sleeve than the windows to increase the discharge velocity through the pilot orifices, and an opening of sufficient size to readily pass silt or contamination particles in the fluid traversing the valve, even at low fluid flow rates. For example, in one embodiment the diameters of the pilot orifices are approximately 0.008 inches, and the discharge windows each have an axial length of approximately 0.080 inches.

In accordance with the invention, a positive fluid pressure differential in the free flow direction of the arrows forces ball 19bb40 to unseat and to be axially displaced (toward the right in FIG. 19BB). The positive pressure differential initially displaces the ball axially until the intersection of the sleeve with the circumference of the ball is axially displaced beyond the pilot orifices 19bb80 and 19bb82. Once the ball uncovers the orifices, the released fluid is initially propelled generally radially through the orifices. As the pressure differential increases, the ball will be further axially displaced to allow a relatively greater volume of fluid flow through the discharge windows 19bb70, 19bb72, 19bb74 and 19bb76. The resulting fluid flow path through the check valve (as illustrated by the broken arrows) extends initially generally axially through the valve seat and then generally radially through the pilot orifices and discharge windows to form a generally unobstructed flow path of annular cross-section between the outer surface of the cylindrical sleeve 19bb24 and the inside wall of the adjacent conduit 19bb12. Consequently, a relatively low resistance to fluid flow can be effectively implemented since the flow path is generally diverted from the region of the spring assembly 19bb42.

In an embodiment, the check valve utilizes two distinct motion variations a) single motion and b) reciprocating motion. The single motion method comprises a single push on the actuator, continuously measuring its displacement until reaching a target value. It is simple and efficient to capture data for one opening/closing cycle. The reciprocating motion method comprises repeatedly pushing and retracting the actuator in a loop, measuring displacement with each cycle, and checking if the desired opening/closing range is achieved. It offers valuable insights into dynamic behavior, wear, and fatigue over time.

Referring to FIG. 19BE, it shows a proof of concept working of device, according to one or more embodiments. The proof-of-concept device was constructed with a microvalve integrated and connected to various components as depicted in FIG. 17BE. The valve operates with a mechanism wherein, in its closed position, an O-ring on one side is pressed against a V-shaped opening by a disk linked to a centered rod, which is pulled towards the opposite side by a compressed spring, with another disk connected to its end. The device can exhibit multiple operational modes: Firstly, when the liquid pressure on either side is lower than the force exerted by the compressed spring, the valve remains closed. Secondly, if the liquid on the O-ring side surpasses the pressure on the spring side, flow through the valve is obstructed. Thirdly, when the liquid pressure on the spring side exceeds both the spring force and the pressure on the O-ring side, the spring compresses, releasing the O-ring seal, enabling liquid flow from the spring side to the O-ring side. Fourthly, when a mechanical force is applied to compress the spring sufficiently, the O-ring seal is released, allowing liquid flow depending on the pressure gradient.

For experimentation, the focus was on fourth operational mode, aiming to exhibit controlled liquid flow through mechanical or electromagnetic forces rather than relying solely on liquid pressures. A microvalve (3.2 mm OD) was positioned within a plastic tube, connecting two tubes through the valve, each having an ID of about 2.5 mm. Inside the tube, on the spring side of the valve, a 2 mm diameter strike rod (15 mm length) was placed, followed by a 2 mm diameter magnet (20 mm length). Adjacent to the junction of the strike rod and magnet, an electromagnetic coil of approximately 10 ohm was positioned outside the tube. When a DC from a 7.5 v battery was supplied to the coil, a magnetic field was generated, aligning with the magnetic field of the magnet to create an S—N attraction, drawing the magnet towards the coil's center. Consequently, the magnet drove the strike rod to compress the spring, opening the valve. Utilizing the pressure from a syringe, liquid is moved from the O-ring side to the spring side.

Considering the effectiveness of momentum during magnet movement when the current was applied compared to stationary magnet attraction pressure, electromagnetic field pulses were employed to generate magnet momentums for valve opening. Thus, controlling the flow rate became achievable by adjusting the pulse rates of the magnet momentums. To minimize power consumption, strategies such as employing a weaker spring or utilizing momentum force rather than conventional force or pressure were suggested.

To evaluate the force necessary for valve operation, two distinct tests were conducted. In test-1, the valve's outlet end was linked to water under 1.15 atmosphere pressure. Simultaneously, the inlet end was connected to a plastic pin, which was affixed to a lever mechanism. By measuring the force required to initiate water flow from the pressurized side, it was determined that approximately 19 grams of force are sufficient to open the valve. In test-2, employing a micro gearbox motor with a 1:700 speed reduction for a helical output shaft, its performance was assessed. The motor can function under a 2.5-kilogram load at 1 volt, drawing 20 μW of power to move a 1-gram weight in alignment with the shaft axis. Based on these tests, it is estimated that <400 μW is necessary to open the 3.2 mm valve. Assuming a 2.5-second duration for both valve opening and closing (totaling 5 seconds per cycle), the energy required for a complete cycle amount to 0.56*10\hat{ }-6 watt-hours (0.56 μWh). To dispense a total volume of 500 μl of liquid by repeatedly opening and closing the valve 25 times, with each cycle delivering 20 μl, the energy demand is 14 μWh. A cell of a voltage range of 3-4.1V and a rechargeable battery boasting of about 2.5 mAh-3 mAh capacity, will generate 8400 μWh of energy at 3V. Remarkably, the charge capacity of the cell exceeds the requirement by 600 times. Alternatively, if the valve remains open for 50 seconds during each cycle (instead of 5 seconds), the charge capacity of the cell still surpasses the need by 60 times.

Referring to FIG. 19BF, it illustrates a valve assembly designed to regulate fluid flow in the device, according to one or more embodiments. The implantable device comprises a valve assembly designed to regulate fluid flow. The valve has three positions: closed, partially open, and fully open. The valve is designed to control the flow of a liquid (presumably medicine) accurately. A spring-loaded valve is located on the left side. The spring provides a slight pressure to keep the valve closed. When pressure is released, the spring pushes the valve shut. A metering tube connects to the valve. A motor generates linear motion, which pushes the metering tube. The metering tube opens the valve, allowing fluid flow. When the valve is fully open, drug flows through the metering tube. A seal prevents leakage between the metering tube and the valve. The motor-driven linear motion controls the valve. By adjusting motor speed, the amount of fluid dispensed can be controlled. The design ensures precise dosing by managing valve opening and closing. The design allows for accurate flow control even under high pressure.

An embodiment relates to a device for a swift, single-dose drug delivery. Referring to FIG. 19BG, the device comprises a drug chamber, a movable plug, an actuator, an outlet for drug releases from the device and an electronic chamber. The drug chamber is filled with a water-soluble drug (for instance, naloxone), water, and carbon dioxide (CO₂) under pressure. The pressurized environment ensures that the drug can be expelled quickly when required. The movable plug serves as a barrier, blocking an outlet hole and keeping the contents of the chamber sealed under pressure. When the delivery of the drug is needed, the plug is retracted. This action unblocks the outlet hole, creating a pathway for the drug solution to be expelled. With the outlet hole unblocked, the pressurized drug solution is released all at once. The high pressure inside the chamber forces the drug solution out rapidly, ensuring immediate delivery. The device is suitable for a swift, single-dose delivery of a water-soluble drug. It ensures that the drug is administered quickly and in a controlled manner, which can be critical in emergency medical situations.

An embodiment relates to a device comprising a pressurized first chamber, a second chamber comprising a drug suspension, a movable plug, an actuator, an outlet for drug releases from the device and an electronic chamber. The pressurized first chamber and the second chamber comprising the drug suspension are separated by a uni directionally movable piston.

In an embodiment, the device uses gas pressure instead of osmotic pressure to push out the drug. Referring to FIG. 19BH, the first chamber is pressurized and contains a mixture of water and CO₂, essential for the subsequent expulsion of the drug. The second chamber holds a suspension of the drug, separated from the first chamber by a piston that maintains the integrity of the two chambers while allowing pressure transmission. The third chamber houses a drug delivery unit equipped with a movable plug, an actuator, responsible for dispensing the drug when activated. The fourth chamber contains an electronic control unit, including a control component to manage the device's operation.

The device operates by using gas pressure to expel the drug, leveraging principles similar to those in asthma inhalers but with enhanced efficiency due to higher pressurization. When activated, the pressurized water and CO₂ in the first chamber force the piston to move. This action pushes the drug from the second chamber into the third chamber and ultimately out through the outlet. The flow of the drug is regulated by the plug that can be adjusted to open or close the outlet, controlling the release of the drug. The sequence of operation involves the pressurized gas forcing the piston to create the necessary force to expel the drug, enhancing the speed and effectiveness of the delivery compared to other methods that rely on osmotic pressure.

An embodiment relates to an implantable device for drug delivery, wherein the pressure is generated via a compressed spring. This device consists of several functional chambers as illustrated in FIG. 19BI. The first chamber contains a compressed spring, which provides the necessary force to expel the drug. The second chamber holds a suspension of the drug, separated from the first chamber by a piston. This piston maintains the separation of the two chambers while allowing the force from the compressed spring to be transmitted. The third chamber houses a drug delivery unit, equipped with a movable plug and an actuator. This unit is responsible for the controlled dispensing of the drug. Additionally, the device includes a fourth chamber containing an electronic control unit, which manages the operation of the device through a control component. The outlet is regulated by a plug that can be opened or closed to control the drug's release. Upon the movable plug opens up, the outlet the drug is released and simultaneously, the energy stored in the compressed spring in the first chamber is released, causing the piston to move. This movement forces the drug from the second chamber into the third chamber and out through the outlet. In this embodiment, the spring's expansion is facilitated only by the presence of compressed gas in the sealed spring chamber. If the chamber contains liquid instead of gas, a small opening in the chamber wall is required to allow liquid to enter, enabling the piston to move. Without such an opening, the piston remains stationary if the chamber is filled with liquid. Alternatively, if the chamber wall features a forward osmosis permeable membrane, the pressure on the piston results from both osmotic pressure and the spring force. Please ensure these details are included in the specification.

### M. Valves for the Device

The various types of valves that can be used for flow control of drug through the implantable device comprise:

### 1. Shape Material Alloy Valve:

A shape-memory alloy (SMA) is an alloy that can be deformed when cold but returns to its pre-deformed (“remembered”) shape when heated. The effect is called “shape memory effect” (SME). SMA material has two phases: a low-temperature phase, which is called martensite; and a high-temperature phase, which is called austenite. It may also be called memory metal, memory alloy, smart metal, smart alloy, or muscle wire.

In an embodiment, shape-memory alloys are at least one of a copper-aluminum-nickel, nickel-titanium (NiTi), and alloys comprising zinc, copper, gold and iron. Although iron-based and copper-based SMAs, such as Fe—Mn—Si, Cu—Zn—Al, and Cu—Al—Ni are commercially available and cheaper than NiTi, NiTi-based SMAs. In an embodiment, the selection of SMA's is based on stability and practicability.

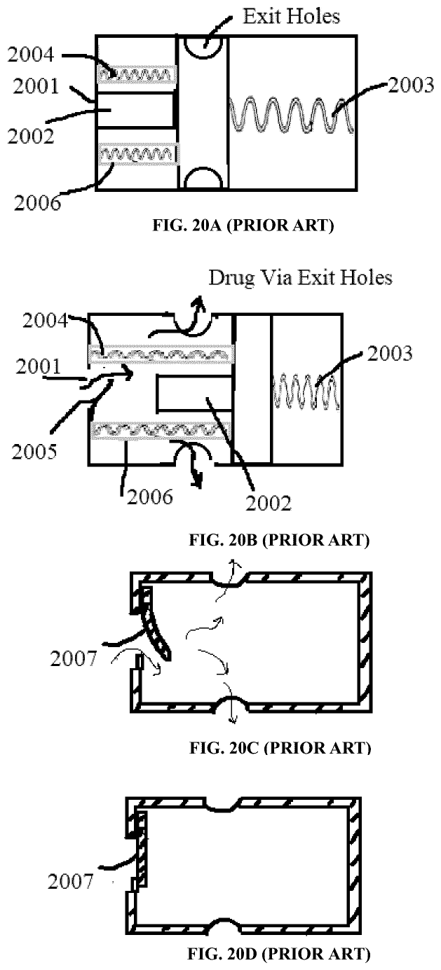

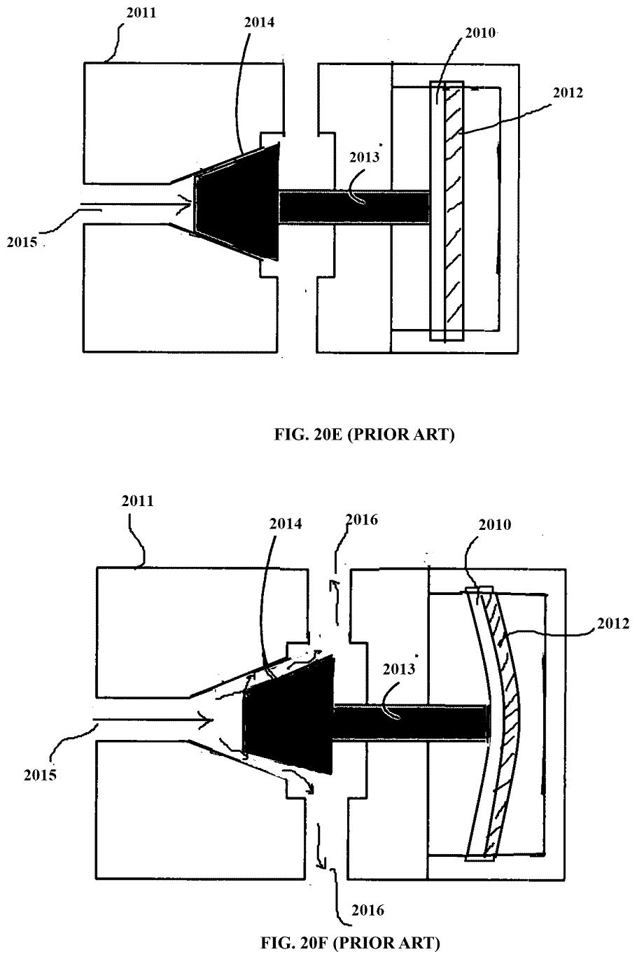

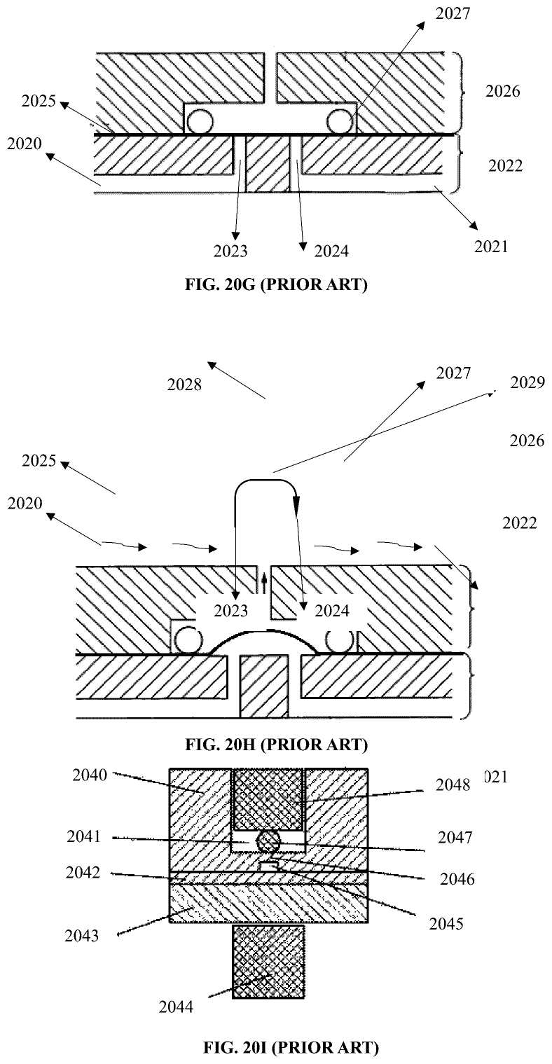

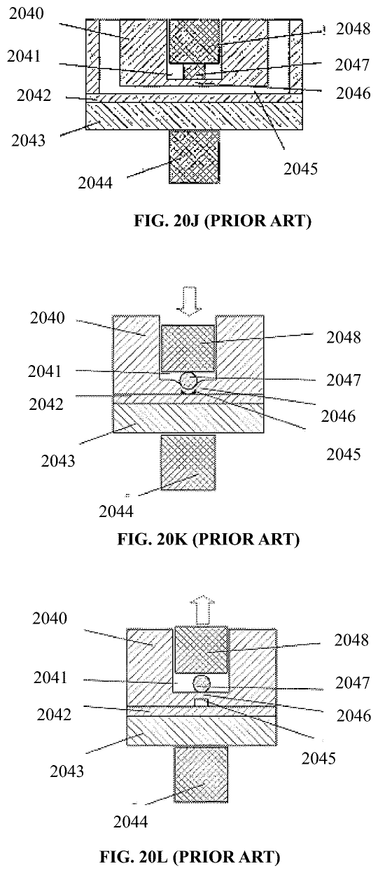

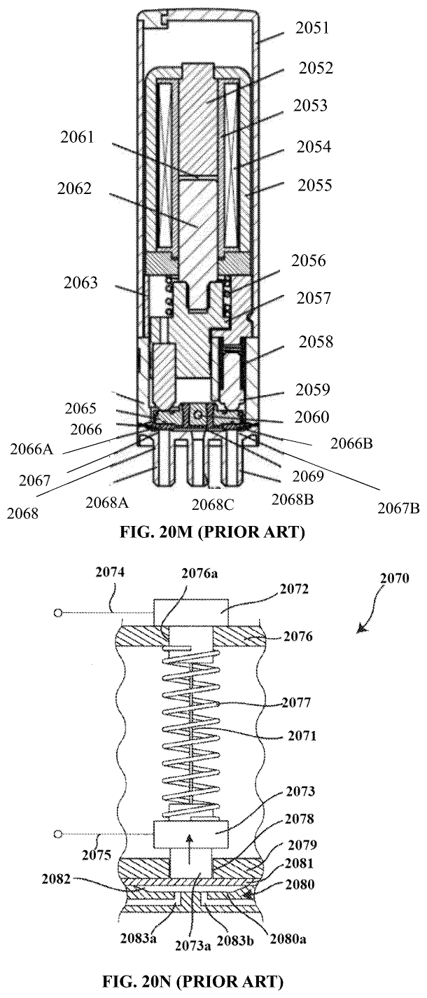

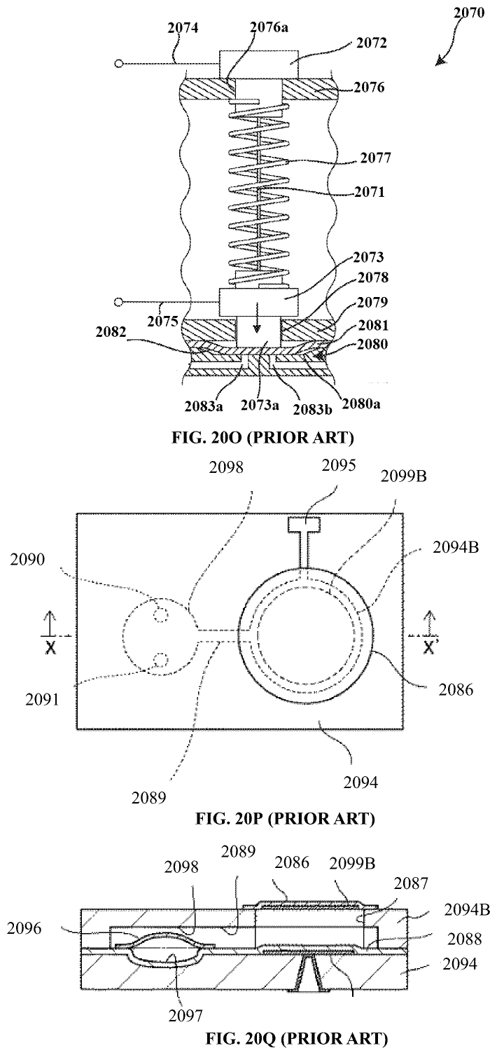

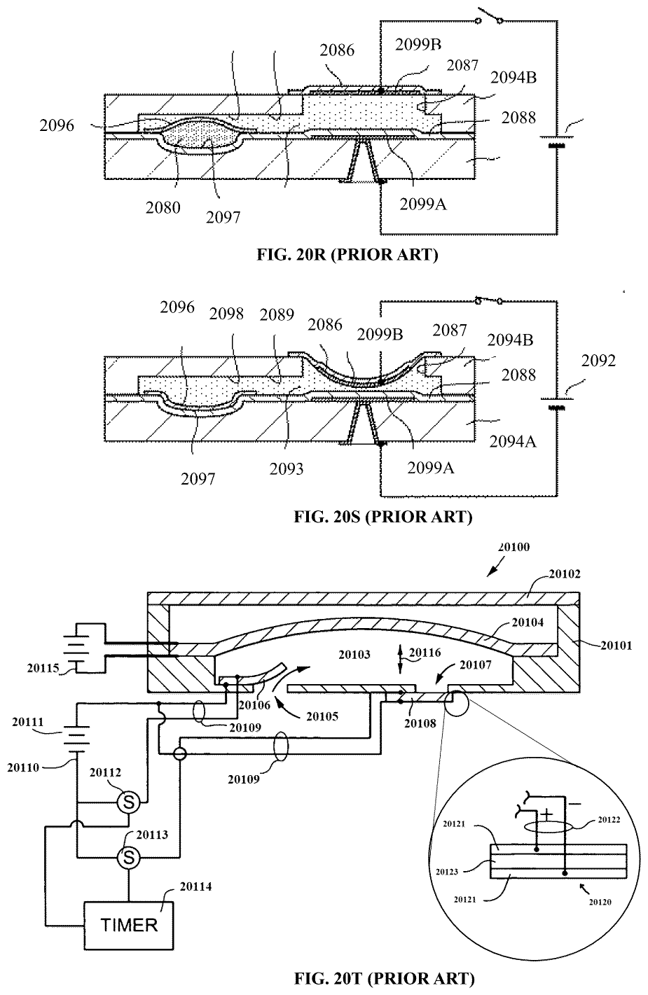

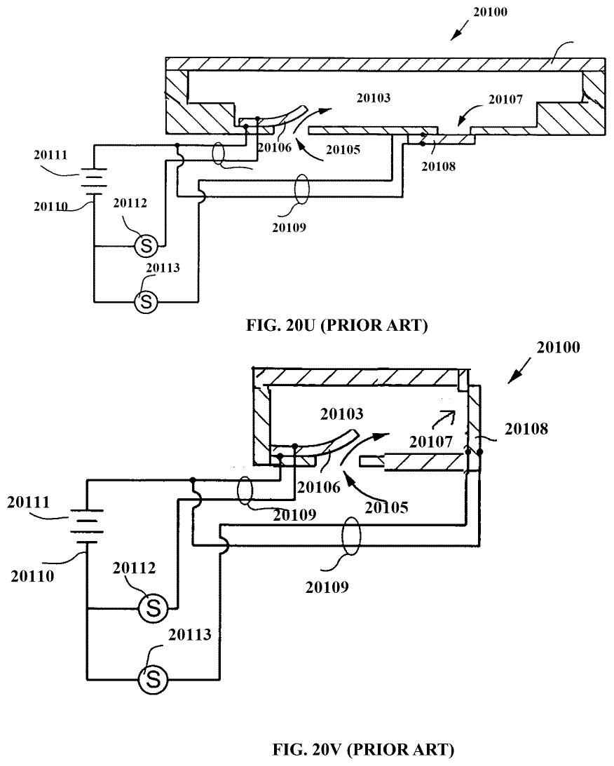

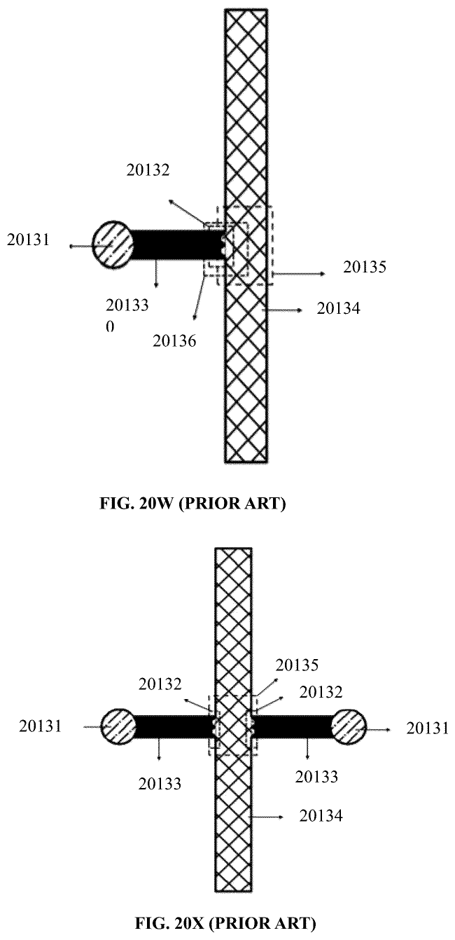

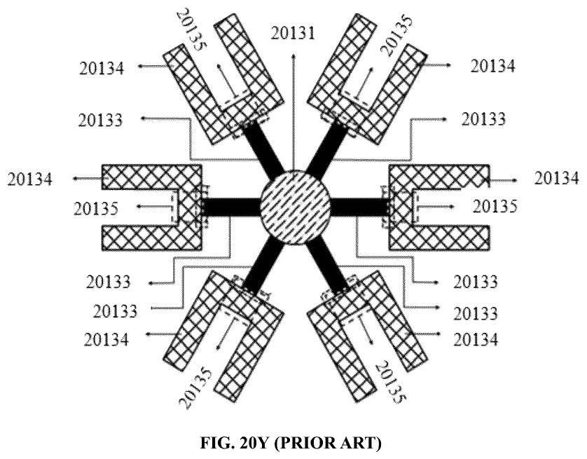

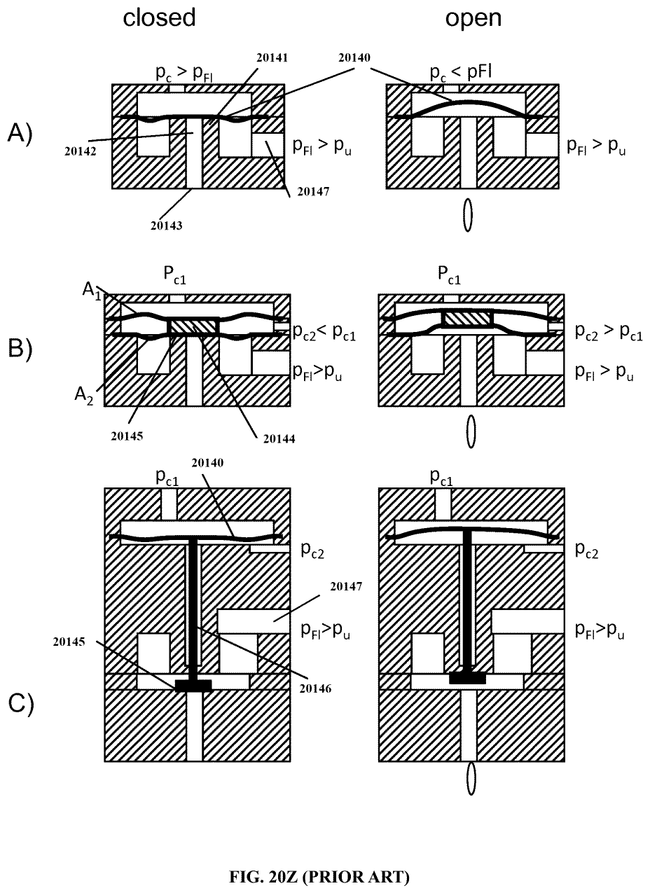

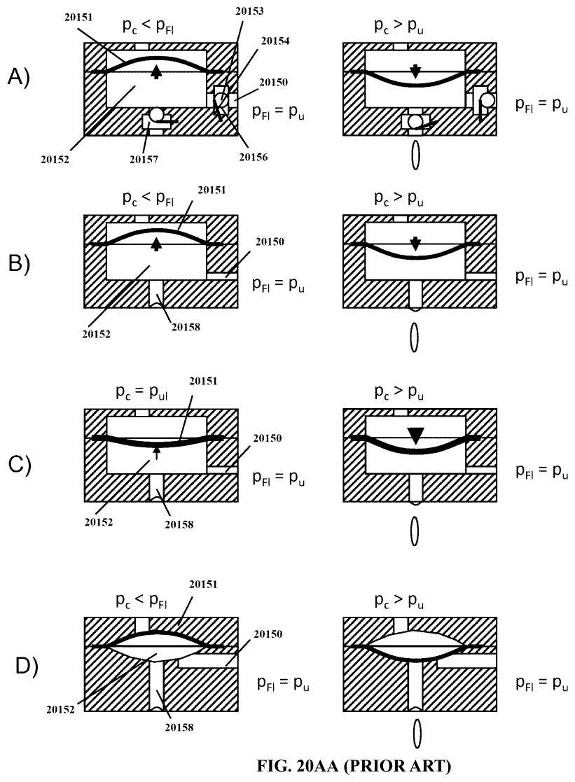

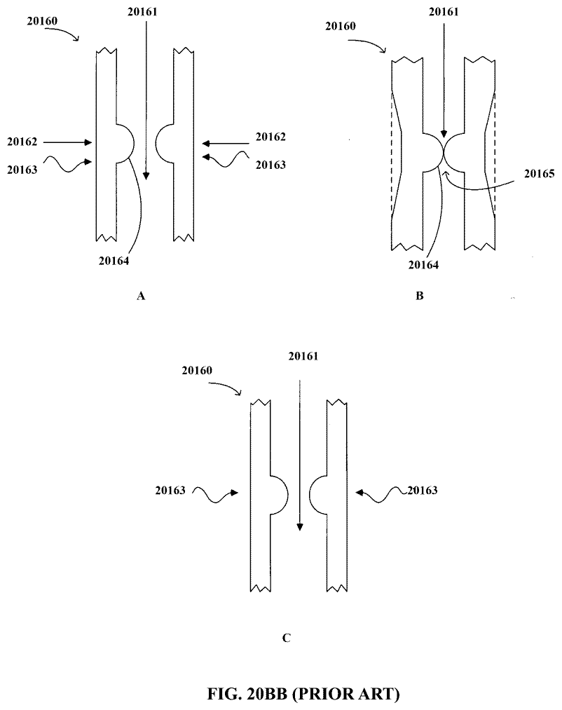

In an embodiment, the valves are designed based on SME using SMAs. In an embodiment, the valve is a normally closed valve. As shown in FIGS. 20A and 20B, the valve comprises a valve seat 2001, a sealing element 2002 that is configured to sit in the valve seat 2001 to seal fluid (drug) within the drug chamber. The valve comprises a spring (a bias spring) 2003, and at least one shape memory alloy element 2004 which is attached to the sealing element 2002 and is movable in response to a temperature increase, and the shape memory effect activated thereby, into a position which opens the valve against an increasing load by the spring 2003. The shape memory alloy element 2004 cooperates with the sealing element and exerts an operating force on the sealing element for closing and opening the valve seat with or against a compression spring force. The valve seat may comprise of a flapper 2005 to avoid fluid flowing back when the spring element 2003 pushes the sealing element 2004 against the valve seat 2001. The SMA element is heated using the battery power from the electronics unit of the implantable device. The SMA element may comprise an enclosure 2006 which is heat resistant and thermally insulative to prevent the heat transfer from the SMA element to the drug. Further, valve seat and the SMA element are designed to be fluid tight.

In another embodiment, the design may comprise of spring and SMA element in concentric manner. Various design aspects and embodiments are envisioned that can be explored based on the SME principle.

FIG. 20C and FIG. 20D show the SMA element 2007 which, when deformed due to SME, opens the valve. Valve seat and the SMA element are designed to be fluid tight.

In an embodiment, the shape memory alloy can be a compression element upon actuation and arranged in a configuration where the valve is in normally closed configuration. In an embodiment, the shape memory alloy can be a Magnetic Shape Memory (MSM) alloy. MSM alloys are ferromagnetic materials that can produce motion and forces under moderate magnetic fields. Typically, MSMAs are alloys of Nickel, Manganese and Gallium (Ni—Mn—Ga).

In an embodiment, the SMA element is selected based on a biocompatible shape memory alloy SMA having a characteristic temperature, that is preferably below body temperature and drug stability temperatures.

In an embodiment, the SMA element can be a compression element and the activation chamber comprising compression SMA element can be on the same side as compression spring 2003 such that there is no heat interaction of SMA element with the drug. Further the activation chamber in such a configuration can be closer to the electronic unit.

### 2. Piezoelectric Valve:

Piezoelectricity is the ability of certain materials to develop an electric charge that is proportional to a direct applied mechanical stress. These materials also can do the opposite, that is they will deform proportionally to an applied electric field. Piezoelectric materials such as crystals and ceramics are nonconductive and non-centrosymmetric. While the piezoelectric effect is natural in certain crystals (quartz, rochelle salts, etc.), it can also be induced in certain ceramics using a process known as poling. In an embodiment, ceramic piezoelectric materials include Lead Zirconium Titanate (PZT) and Barium Titanate.

According to an embodiment, piezoelectric actuator configuration can be a piezoelectric stack, or multilayer actuators manufactured by stacking up piezoelectric disks or plates, the axis of the stack being the axis of linear motion that occurs when a voltage is applied. In another embodiment, piezoelectric actuator may be a tube actuator, which are monolithic devices that contract laterally and longitudinally when a voltage is applied between the inner and outer electrodes. In another embodiment, the piezoelectric actuator is a disk actuator which may be in the shape of a planar disk. In another embodiment, it may be a ring actuator, which are disk actuators with a center bore, making the actuator axis accessible for optical, mechanical, or electrical purposes. Other less common configurations include block, disk, bender, and bimorph styles can also be designed for the purpose. In another embodiment, the actuators are programmable Piezo Shims, which are similar to piezo stack actuators but do not require a continuous voltage source to hold a position once programmed.

According to an embodiment, the piezoelectric actuators can be direct. In another embodiment, the actuators are amplified. The effect of amplification may result in larger displacement.

FIG. 20E and FIG. 20F depict a diagrammatic side view of an example, non-limiting embodiment of a piezoelectric valve.

According to an embodiment, piezoelectric valve comprises valve body. Valve body has an actuator housing section 2010 and a flow restrictor housing section 2011. The actuator housing section 2010 at least partially encloses a piezoelectric actuator 2012 which, in the example implementation, may be a strip comprising piezo ceramic and a passive/conductive substrate. Piezoelectric actuator 2012 is connected to unillustrated valve drive circuitry. It will be appreciated that other structures suitable for the piezoelectric actuator of valve, including stack types linear of piezoelectric actuators, disc type, tube type etc. may be used, and the design can be modified as per the actuator action and position. The piezoelectric actuator 2012 is attached or affixed to a stem 2013 of flow restrictor 2014. Flow restrictor 2014 has a head which, in the illustrated embodiment, takes the form of a truncated cone. Depending on the degree of actuation of piezoelectric actuator 2012, the flow restrictor head may bear against and thereby block a passage 2015 internally formed in flow restrictor housing section 2011, or may be spaced away from passage 2015, as shown in FIG. 20F, in order to permit fluid flow between an input port (flow passage 2015) and an output port 2016 formed in flow restrictor housing section 2011.

The valve may be designed as a flat valve, or a cylindrical valve based on the geometry and space requirements.

### 3. Micro Fluidic Valve:

FIG. 20G illustrates a cut outside view of the diaphragm valve. The first pathway 2020 and the second pathway 2021 are configured within a rigid body 2022. The inlet end 2023 and the outlet end 2024 are closed by the valve membrane, which is part of a membrane cover 2025, which is a cover for the rigid body 2022. The membrane cover 2025 includes at least two layers, a top layer and a bottom layer. The top layer has a higher thermal melting point than the bottom layer. The bottom layer is placed directly above the rigid body 2022. For example, the top layer is polyester, and the bottom layer is polypropylene. In general, the membrane cover 2025 is a flexible material that includes at least an adhering layer and a high melting temperature top layer. The membrane cover 2025 can also include additional layers in between the adhering layer and the high melting temperature layer. A manifold 2026 is coupled to the diaphragm valve such that a bottom surface of the manifold 2026 is in contact with the top surface of the diaphragm valve. The manifold 2026 includes an O-ring 2027 configured to be positioned against the top surface of the membrane 2025. The O-ring 2027 is also configured to have a perimeter greater than distance between the inlet valve and the outlet valve, thereby forming the valve membrane 2025 within the perimeter of the O-ring 2027.

FIG. 20H illustrates the diaphragm valve actuated to an open position. To open the diaphragm valve, a vacuum is applied via the air passage 2028. The vacuum forces the valve membrane 2025 away from the recessed valve area 2029, thereby opening the fluid pathway between the fluid inlet end 2023 and the fluid outlet end 2024. In this case, application of a vacuum is not necessary. Alternatively, a vacuum can be applied to improve the volume of the fluid pathway.

In an embodiment, the valve may be a mechanically-actuated microfluidic valve. The valve comprises a thermal bend actuator as elaborated in U.S. Pat. No. 7,981,386B2, titled, “Mechanically-actuated microfluidic valve”.

In an embodiment, the valve may be a mechanically-actuated microfluidic diaphragm valve. The valve comprises a thermal bend actuator as elaborated in U.S. Pat. No. 8,092,761B2, titled, “Mechanically-actuated microfluidic diaphragm valve”.

### 4. Electromagnetic Valve:

The microvalve is an important part of the microfluidic chip device, which is mainly used to adjust the fluid flow size, the opening and closing of the fluid channel and the switching of the fluid flow direction. In various microfluidic systems, microvalves have a wide range of applications. For example, microchemical systems, bioanalytical systems, micropumps, etc. all require the use of microvalves.

In an embodiment, the microvalve can be a centre lathe type electromagnetic micro valve structure be integrated on micro-fluid chip. The working principle of the electromagnetic microvalve is as follows: the electromagnetic microvalve controls the fluid in a manner of “down pressing” to energize the electromagnet, a magnetic field is generated around the electromagnet, attracting the permanent magnet, and the thimble reacts (is pressured) according to the strength of the magnetic attraction. The suction between the two sides presses the PDMS elastic film on the bottom surface of the valve seat, thereby realizing the function of the electromagnetic microvalve to block the movement of the fluid; when the channel needs to be opened, the current direction is changed, so that the electromagnet generates a reverse magnetic field, and the permanent magnet is in the repulsive force. After moving up, the elastic film moves up to open the channel under the action of elastic force.

The micro-fluidic chip includes a fluid passage and substrate, it is characterized in that: the micro-valve comprises a slice permanent magnet, electromagnet, a thimble and a valve seat. The valve seat is the lower shrinkage pool being machined on the chip and being positioned at above fluid passage. The valve seat bottom surface comprises Polydimethylsiloxane (PDMS) elastic film. The bottom surface of the valve seat is a common wall of the valve seat and fluid passage. The upper end of valve seat is connected with the external world. The thimble is arranged at valve seat bottom surface center position and is positioned directly over described fluid passage. The permanent magnet is in valve seat and positioned directly over thimble. The electromagnet is arranged below the substrate and is positioned below the permanent magnet. The electromagnetic micro valve, described herein, can automatically, fast respond, and has simplicity of design, and precise operation control, is applied widely, can automatically sequence, and realizes the break-make of special modality fluid transport.

As shown in FIG. 20I and FIG. 20J, the electromagnetic microvalve integrated on the microfluidic chip provided by the present invention comprises a permanent magnet 2048, an electromagnet 2044, a thimble 2047 and a valve seat 2041. The microfluidic chip comprises two layers, the upper layer is a PDMS chip 2040 and the lower layer is a substrate 2043; the substrate 2043 is spin-coated with a PDMS coating 2042, the spin coating thickness is 0.5-1 mm; the bottom surface of the valve seat 2041 is a PDMS elastic film 2046, and the bottom surface is a common wall of the valve seat 2046 and the microfluidic chip fluid passage 2045, the elastic film may have a thickness of 500 μm; the upper end of the valve seat is connected to the outside; the thimble 2047 is located at the PDMS elastic. On the film 2046, corresponding to the center position of the valve seat 2041, directly above the fluid passage 2045, the permanent magnet 2048 is placed in the valve seat 2041, directly above the fluid passage 2045, pressed against the ejector pin; the electromagnet 2044 is placed under the substrate 2043, facing the permanent magnet 2048.

The thimble may be in the shape of a cylinder or a sphere, and the material may be selected from a high elastic modulus metal material such as steel or iron; the size thereof depends on the size of the fluid channel of the microfluidic chip, and a preferred example is For the cylindrical thimble, when the fluid passage width is 80 μm and the height is 30 μm, a cylindrical seat thimble having a diameter of 500 μm and a height of 1-2 mm can be selected.

The permanent magnet is located in the valve seat, and a neodymium iron boron permanent magnet is used; the shape of the permanent magnet depends on the shape of the valve seat, generally a cylinder or a rectangular parallelepiped; the size of the bottom surface of the permanent magnet is smaller than the valve seat 0-0.5 Mm, but not 0.

The electromagnetic micro-valve provided by the invention realizes the on-off control of the fluid passage by the magnetic force between the electromagnet 2044 and the permanent magnet 2048. As shown in FIG. 20K, when the electromagnet 2044 is energized, the permanent magnet 2048 acts under the action of the magnetic field. In the lower movement, the permanent magnet presses the thimble 2047 to move the ejector pin downward and transmits the pressure to the PDMS elastic film 2046, causing the elastic film 2046 to undergo corresponding elastic deformation, which causes the elastic film 2046 to be in close contact with the fluid passage 2045, limiting fluid passage as the permanent magnet presses the thimble downward. When the electromagnet 2044 is applied with a reverse voltage, the inner wall causes the fluid passage 2045 to be in a completely open state; as shown in FIG. 20L, the permanent magnet 2048 moves upward under the action of the reverse magnetic field, lifting the ejector pin, the thimble, and the elastic film 2046 is under the action of elasticity. Returning to the initial unstressed state, the fluid passage 2045 is opened, and the fluid passage 2045 is in a full passage state; therefore, the on/off control of the fluid passage can be accurately realized by the magnetic force between the electromagnet and the permanent magnet.

When the electromagnetic micro-valve provided by the present invention realizes the control of the fluid direction, the control of the fluid direction in the microfluidic chip can be realized by selectively controlling the current direction of the electromagnet by providing a plurality of electromagnetic microvalves.

In the above electromagnetic microvalve, the thickness of the PDMS elastic film 2046 can be adjusted in the range of 0-500 μm; by controlling the current in the electromagnet line, the flow velocity of the fluid in the flow channel can also be accurately controlled; The position of the iron and permanent magnets can also be interchanged, as well as the result of controlling the on-off and flow.

The above is only a preferred embodiment of the present invention, but the scope of the present invention is not limited thereto, and any technical person skilled in the art within the technical scope disclosed by the present invention, the technical solution according to the present invention equivalent substitutions or modifications of the inventive concept are intended to be included within the scope of the invention.

### 5. Micro Solenoid Valve:

Shown in FIG. 20M, the micro-electromagnetic valve utilizes the action of electromagnetic force to drive the valve mechanism by the movement of the movable iron core 2062, so as to switch the flow passage of the fluid in the valve body 2064, which includes a valve body 2064, a valve seat 2067, a diaphragm 2066 isolating the valve seat 2067 from the valve body 2064, and a solenoid part and a valve mechanism arranged in the valve body 2064; the solenoid part comprises a fixed iron core 2052, the movable iron core 2062 and the electromagnetic coil 2054, the valve mechanism includes a first pressurizing member 2057, a rotating member 2065 and a second pressurizing member 2059, the first pressurizing member 2057 is connected to the movable core 2062, and finally the multiple ports 2068A, B, and C.

The micro-electromagnetic valve of the present invention is provided with a solenoid part coaxially distributed with the valve body 2064, the valve mechanism and the port 2068, a first pressurizing part 2057 connected with the movable core 2062 and driven by a first pressurizing member 2057. The spring 2056 is pressed to move the first pressurizing member 2057 in the direction of the opening 2068 and the first pressurizing member 2057 drives the second pressurizing member 2059 to rotate relative to the first pressurizing member 2057 by the rotating member 2065 rotating in a circle around the fixed shaft. The pressurizing member 2057 is moved in the opposite direction in parallel to realize the state transition of the one-side port, and the switching of the other port is achieved by reversing both the previous moving direction, that is, the first pressurizing member 2057 and the second pressurizing member 2059. The member 2059 is driven by the rotating member 2065 rotating around the fixed shaft to rotate in a circle and the first pressurizing member 2057 and the second pressurizing member 2059 are moved in parallel in the opposite directions to switch the state of the port 2068. The first pressurizing member 2057 and the second pressurizing member 2059 are interlocked by the rotating member 2065 rotating around the fixed shaft in a rotating manner and are in rigid contact and rigid transmission therebetween. The first pressurizing member 2057 and the second pressurizing member 2059 is relatively synchronized in movement so that the valve mechanism has a short response time.

The basic operation principle of the three-way valve is described below.

As shown in FIG. 20M, the solenoid part includes the following components: a holding device 2055 disposed inside the housing 2051; a fixed iron core 2052 having a cylindrical structure and mounted to the upper surface of the holding device 2055; an electromagnetic coil 2054. The coil 2054 is externally mounted on the fixed iron core 2052 through the bobbin 2053; the movable iron core 2062 is movably disposed in the hole formed by the bobbin 2053, the movable iron core 2062 and the fixed iron core 2052 are axially aligned where a predetermined gap 2061 is arranged.

The valve seat 2067 and the valve body 2064 are fastened by screws, the valve seat 2067 and the valve body 2064 are sealed by the diaphragm 2066, and the cavity 2069 is formed between the diaphragm 2066 and the valve seat 2067.

As shown in FIG. 20M, when no electric power is applied to the electromagnetic coil 2054, the movable core 2062 is separated from the fixed core 2052 by the urging force of the first pressurizing spring 2056 to form a predetermined gap 2061 therebetween. The first pressurizing member 2057 slides downward and the first pressurizing member 2057 exerts a force on the diaphragm sheet 2066 with the first diaphragm sheet expanded portion 2066A thereof placed on the first plane expanded structure 2067A of the valve seat 2067 such that the first port 2068A is closed, and correspondingly, the second port 2068B and the third port 2068C are in a connected state. This state is the initial state of the three-way valve, the initial state of the partial cross-sectional view of an enlarged view.

When a current flows through the electromagnetic coil 2054, the electromagnetic coil 2054 is energized to generate a suction force, so that the movable iron core 2062 overcomes the force of the first pressurizing spring 2056 and is attracted to the fixed iron core 2052 to drive the first pressurizing member 2057 upward away from the valve seat 2067, and accordingly, the second pressurizing member 2059 moves downward so that the rotating member 2065 rotates clockwise and the diaphragm 2066 coupled therewith moves, and the second diaphragm on the diaphragm 2066 expands The portion 2066B is placed on the second planar expansion structure 2067B on the valve seat 2067, the second port 2068B is closed, the first port 2068A and the third port 2068C are in a communication state, so as to complete the sealing of the other side channel, and complete port flow channel switching. Here, the removal of the residual liquid in the cavity 2069 is achieved by switching the state.

### 6. Micro Valve:

FIG. 20N shows an outline of a driving mechanism in an extended state, the driving mechanism uses shape memory alloy wire 2071 that drives microvalve 2070 in an embodiment of the present disclosure. FIG. 20O shows an outline of the driving mechanism in a contracted state, the driving mechanism uses shape memory alloy wire/s that drives the microvalve 2070 in an embodiment of the present disclosure. In the driving mechanism in FIGS. 20N and 20O, a representative example of shape memory alloy wire 2071 will be described.

In FIG. 20N and FIG. 20O, shape memory alloy wire 2071 is held by holding member 2072 at one end, and is held by pressing member 2073 at another end. Wiring 2074 is also held by holding member 2072, and shape memory alloy wire 2071 and wiring 2074 are electrically connected inside holding member 2072. Wiring 2075 is also held by pressing member 2073, and shape memory alloy wire 2071 and wiring 2075 are electrically connected inside pressing member 2073.

Holding member 2072 is joined to an inside of through-hole 2076a of fixed plate 2076 to be fitted and fixed, and is fixed to fixed plate 2076 along a surface of fixed plate 2076 so as not to be displaced (in a right-left direction in FIGS. 20N and 20O). Compression spring 2077 is disposed in a compressed state between fixed plate 2076 and pressing member 2073 with shape memory alloy wire 2071 linearly penetrating a central portion of compression spring 2077. Thus, compression spring 2077 is configured to apply a tension in an extension direction to shape memory alloy wire 2071 by a biasing force of compression spring 2077.

End portion 2073a having a small diameter of pressing member 2073 is fitted and disposed so as to be able to freely perform reciprocation operation inside hole 2078 provided in pressure plate 2079 disposed, for example, parallel to fixed plate 2076. End portion 2073a of pressing member 2073 can reciprocate between a projection position where end portion 2073a projects from hole 2078 of pressure plate 2079, and a withdrawal position where end portion 2073a is withdrawn inside hole 2078 of pressure plate 2079.

Pressure plate 2079 and fixed plate 2076 are restrained lest mutual relative positions should change. Moreover, pressure plate 2079 is fixed such that an outer surface thereof (a lower surface in FIGS. 20N and 20O) pushes flow channel substrate 2080 through resin film 2081, and the biasing force of compression spring 2077 prevents pressure plate 2079 from floating up from resin film 2081.

Thus, resin film 2081 is held cohesively to pressure plate 2079. As one example, resin film 2081 can be made of silicon rubber, acryl resin with a thin portion functioning as a hinge, or the like.

Moreover, in a portion of flow channel substrate 2080 opposed to hole 2078 of pressure plate 2079, depression 2080a to form internal space 2082 is provided, and in the projection position where end portion 2073a of pressing member 2073 projects from hole 2078 of pressure plate 2079, end portion 2073a presses resin film 2081 and enters depression 2080a to cause resin film 2081 to adhere to a bottom surface of depression 2080a of flow channel substrate 2080.

Flow channel substrate 2080 has inlet-side flow channel 2083a and outlet-side flow channel 2083b connected to inlet-side flow channel 2083a through internal space 2082, and an opening of inlet-side flow channel 2083a and an opening of outlet-side flow channel 2083b are exposed to internal space 2082, and both the openings are simultaneously opened and closed by resin film 2081.

That is, when resin film 2081 is caused to adhere to the bottom surface of depression 2080a of flow channel substrate 2080, the opening of inlet-side flow channel 2083a and the opening of outlet-side flow channel 2083b are sealed by resin film 2081 to cut off inlet-side flow channel 2083a and outlet-side flow channel 2083b. On the other hand, when the adhesion of resin film 2081 to the bottom surface of depression 2080a of flow channel substrate 2080 is released, the opening of inlet-side flow channel 2083a and the opening of outlet-side flow channel 2083b are released, so that a fluid can be caused to flow from inlet-side flow channel 2083a to outlet-side flow channel 2083b.

When shape memory alloy wire 2071 is not subjected to energization heating, shape memory alloy wire 2071 enters the extended state as shown in FIG. 20N, and end portion 2080a of pressing member 2080 is pushed to resin film 2081 by the biasing force of compression spring 2077. At this time, resin film 2081 is deformed until the resin film 2081 comes into contact with the bottom surface of depression 2080a of flow channel substrate 2080, so that a volume of internal space 2082 surrounded by resin film 2081 and flow channel substrate 2080 becomes minimal, and the connection between flow channels 2083a 2083b and internal space 2082 is cut off. On the other hand, when shape memory alloy wire 2071 is subjected to energization heating, shape memory alloy wire 2071 enters the contracted state against the biasing force of compression spring 2077, as shown in FIG. 20O, and pressing member 2073 is displaced upward in FIG. 20O by a force of shape memory alloy wire 2071. End portion 2073a of pressing member 2073 is located at the withdrawal position inside hole 2078 of pressure plate 2079 in accordance with a displacement amount of pressing member 2073, and the deformation of resin film 2081 is released, thereby releasing the sealing of flow channels 2083a, 2083b by resin film 2081. Furthermore, the volume of internal space 2082 is increasing, and flow channels 2083a, 2083b and internal space 2082 come to connect.

The microvalve 2070, the state in FIG. 20N is a valve-closed state where flow channel 2083a and flow channel 2083b are cut off, and the state in FIG. 20O is a valve-opened state where flow channel 2083a and flow channel 2083b are connected.

In an embodiment, the micro valve may be made using shape memory alloy with flat geometry and using similar design elements as disclosed in U.S. Ser. No. 10/935,152B2 titled, “Valve having an actuator made of a shape memory alloy, with a flat geometry” and US111136968B2, titled, “Actuator assembly” which are incorporated herein in their entirety.

### 7. Electrostatic Microvalve:

In one embodiment, the microvalve can be an electrostatic microvalve. The electrostatic microvalve is formed on a substrate, a sample flow path for flowing a sample fluid, and formed on the substrate, the sample flow path is separated by an elastic film. A drive tank opens and closes the sample flow path by deforming an elastic film, and a pressurized tank formed on the substrate and connected to the drive tank by a flow path, by changing the volume of the pressurized tank. A driving elastic film that changes the pressurization of the working fluid, and a pair of electrodes including an electrode supported by the driving elastic film and deformable together with the driving elastic film, and an electrode fixed to face the electrode. A pressure vessel for deforming the driving elastic film by electrostatic force between the pair of electrodes.

The micropump utilizes the electrostatic microvalve. In one embodiment, three of the electrostatic microvalves are arranged so that their sample flow paths are connected in series, and the electrostatic microvalve at both ends is used. The opening/closing timing of the microvalve may change, and the liquid supply in the sample flow path is controlled by the opening/closing of the central electrostatic microvalve.

Another aspect of the micropump is such that a plurality of the electrostatic microvalves are arranged so that their sample flow paths are connected in series, and the electrostatic microvalves are sequentially closed along the sample flow paths. By controlling the opening and closing of the electrostatic microvalve, the liquid is sent through the sample flow path.

In one embodiment, In the electrostatic microvalve, at least one of the pair of electrodes of the pressurized tank is not planar, and a part of the pair of electrodes is in contact with each other with an insulating film interposed therebetween in a state where voltage is not applied to both electrodes. Alternatively, the voltage may be applied to close the gap between the electrodes from that portion. In that case, the pressurized tank can be driven with a small, applied voltage. The electrode may be divided into a plurality of parts, and the closed state from a partially closed state to a fully closed state may be controlled by adjusting the number of electrode parts to which a voltage is applied.

In an aspect of electrostatic micropump, the electrode of the central electrostatic microvalve may be divided into a plurality of portions, and the flow rate of the liquid may be controlled by adjusting the number of electrode portions to which a voltage is applied.

FIG. 20P and FIG. 20Q are diagrams showing an electrostatic microvalve according to one embodiment. FIG. 20P is a schematic plan view, and FIG. 20Q is a cross-sectional view taken along the line XX′. The substrate is formed by joining the lower substrate 2094A and the upper substrate 2094B; a synthetic quartz glass substrate, a Pyrex (registered trademark) glass substrate, another glass substrate, a silicon substrate, or the like can be used as a material.

The sample flow path 2097 is formed as a recess on the surface of the lower substrate 2094A, and an inlet 2090 (shown in FIG. 20P) for introducing a sample liquid into the sample flow path and an outlet 2091 for discharging the sample liquid are connected from the flow path 2097 to the back surface of the substrate 2094A.

The drive tank 2098 is formed on the sample flow path 2097 by the upper substrate 2094B, and the drive tank 2098 and the sample flow path 2097 are separated by the elastic film 2096. The driving tank 2098 is constituted by a recess formed in the upper substrate 2094B. When the elastic membrane 2096 is not pressurized by the working fluid, the elastic membrane 2096 is flat and does not obstruct the flow of the sample in the sample flow path 2097, and when the elastic membrane is deformed so as to be pressurized by the working fluid, the sample flow path 2097 is obstructed. The flow of the sample in the sample flow path 2097 is stopped. Thus, the sample channel 2097 is opened and closed by the elastic film 2096.

A pressure vessel 2087 is formed at a position distant from the sample flow path 2097 and the driving vessel 2098 by a concave portion of the upper substrate 2094B and a surface of the lower substrate 2094A. An elastic film 2086 is provided in the pressurizing tank 2087 so that the volume of the pressurizing tank 2087 can be changed, and a thin film electrode 2099b which is deformable together with the elastic film 2086 is provided inside the elastic film 2086. FIG. 20P denotes a terminal 2095 connected to the electrode 2099b, which is connected to an external power supply via the terminal 2095. The other thin-film electrode 2099a is fixed to the wall surface of the pressure vessel 2087 facing the electrode 2099b, that is, the surface of the lower substrate 2094A, and the upper part of the electrode 2099a is the surface inside the pressure vessel 2087 and is insulated. The pressure vessel is covered with the film 2088. The presence of the insulating film 2088 prevents electrical contact between the electrodes 2099a and 2099b even if the elastic film 2086 is deformed and the electrode 2099b is deformed toward the electrode 2099a. Although a terminal for connecting the electrode 2099a to an external power supply device is not shown, it is possible to take out the terminal through, for example, a bonding surface between the substrates 2094A and 2094B or to form a through hole in the substrate 2094A.

As for the elastic films 2096 and 2086, a silicone resin film having a thickness of several tens μm to several hundred m, a fluorine amorphous resin (for example, CYTOP: manufactured by Asahi Glass Co., Ltd.), PDMS (polydimethylsiloxane), or the like can be used. It is desirable that the structural material, other than the elastic films 2096 and 2086, has a small elastic deformation. For example, the structural material may be made of the same material as the elastic films 2096 and 2086 and may be manufactured integrally with the elastic films 2096 and 2086.

As for the electrodes 2099a and 2099b, gold, platinum, or the like can be used as a thin film having a thickness of several tens to several hundreds of nm.

The pressurized tank 2087 is connected to the drive tank 2098 by a flow path 2089, and a working fluid 2093 is filled into the drive tank 2098; the pressurized tank 2087, and the flow path 2089 connecting between them. The working fluid 2093 is preferably an insulating liquid such as silicone oil.

Next, the operation of this embodiment is described. FIG. 20R shows a state in which no voltage is applied to the electrodes 2099a and 2099b of the pressurized tank 2087 and a sample can flow through the sample flow path 2097. FIG. 20S shows a state in which a voltage is applied from the power supply device 2092 to the electrodes 2099a and 2099b of the pressurized tank 2087. By applying a voltage to the electrodes 2099a and 2099b, an electrostatic attractive force acts between the electrodes 2099a and 2099b, the elastic film 2086 is deformed, and the volume of the pressurizing tank 2087 is reduced. As a result, the working fluid 2093 is sent from the pressurized tank 2087 to the drive tank 2098 via the flow path, the volume of the drive tank 2098 expands, and the elastic film 2096 is deformed. As a result, the sample channel 2097 is closed. Since the insulating layer 2088 exists between the electrodes 2099a and 2099b, no short circuit occurs between the electrodes 2099a and 2099b.

### 8. Active Micro Valve:

FIG. 20T, FIG. 20U and FIG. 20V are described generally, and as such are meant to be representative of many different device configurations which can host the inventive advancement described herein. Device 20100 comprises a body which includes a device body base 20101 and a device body lid or cover 20102. For the particular geometry shown in FIG. 20T, the device body, including both its device body base 20100 and a device body cover 20102, are essentially cylindrical (e.g., circular as seen from the top). A pumping chamber 20103 is formed in the device body, and an actuator is provided for drawing fluid into pumping chamber 20103 and pumping fluid out of pumping chamber 20103.

It just so happens that the form of the actuator illustrated in FIG. 20T is a diaphragm 20104. However, it should be understood that, for this and subsequently described embodiments, the actuator need not be a diaphragm but could take other forms such as, for example, a piston-type actuator or even a peristaltic type of actuator. In the particular case that the actuator is actually a diaphragm, the diaphragm 20104 can be clamped, adhered, fastened, or welded, preferably about its periphery, to a seat or other surface of the device body.

The device body 20101 of the example device 20100 of FIG. 20T has an inlet port 20105 which is selectively opened and closed by inlet valve 20106 with which it is aligned. Similarly, device body 20101 has an outlet port 20107 which is selectively opened and closed by outlet valve 20108, the outlet valve 20108 being aligned or situated for opening and closing of outlet port 20107. The inlet valve 20106 admits the fluid into the pumping chamber 20103, whereas the outlet valve 20108 permits fluid to be discharged from the pumping chamber 20103. In the embodiment of FIG. 20T, both of the valves 20106 and 20108 are active valves in that they are actively driven, e.g., by an external signal or circuit, and are not merely passively responsive to phenomena (e.g., fluidic phenomena) occurring in the pumping chamber 20103.

The valves of device 20100 (e.g., either inlet valve 20106 or outlet valve 20108) comprise a deformable or flexible member which is a piezoelectric member (e.g., piezoceramic film). That is, one or both of valves 20106, 20108 comprise a piezoelectric element that preferably constitutes a working portion of the valve. As explained subsequently, the piezoelectric member comprising the valve preferably has electrodes sputtered or otherwise formed on its opposing major surfaces.

In whatever form it takes, application of a voltage to piezoelectric element causes a flexure, stress, or compression in a piezoelectric wafer which comprises piezoelectric element. The flexure, stress, or compression in piezoelectric wafer causes the piezoelectric element to deflect or displace, thereby moving the valve which it comprises, either to a port closing position or to a port opening position. In the particular implementation shown in FIG. 20T, application of a non-zero voltage to the valve causes flexure of the piezoelectric element and thus an opening of the port that otherwise would be covered by the valve, i.e., normally a closed valve.

As illustrated in inset of FIG. 20T, the piezoelectric element 20120 (which can be included in inlet valve 20106 and/or outlet valve 20108) has thin electrodes 20121 sputtered or otherwise is formed on its two opposing major surfaces. The electrodes 20121 can be formed of Nickel or Silver, or other appropriate conductive metal. One of the electrodes 20121 is a positive electrode; the other electrode 20121 is a negative electrode. The positive and negative electrodes 20121 are engaged by respective positive and negative leads 20122.

The piezoelectric element 20120 preferably comprises a multi-layered laminate. The multi-layered laminate can comprise a piezoelectric wafer 20123 which is laminated by an adhesive between an unillustrated metallic substrate layer and an unillustrated outer metal layer. The structure of the multi-layered laminate and a process for fabricating the same are described in one or more of the following (all of which are incorporated herein by reference in their entirety): PCT Patent Application PCT/US01/28947, filed 14 Sep. 2001; U.S. patent application Ser. No. 10/380,547, filed Mar. 17, 2003, titled “Piezoelectric Actuator and Pump Using Same” U.S. patent application Ser. No. 10/380,589, filed Mar. 17, 2003, titled “Piezoelectric Actuator and Pump Using Same”.

The piezoelectric element can be mounted to, affixed to or on, or incorporated into the valve in various ways. In an embodiment, the valve 20106 has a shoulder portion which is proximate a sidewall of device body 20101, and a distal portion which flexibly extends over inlet port 20105. At its shoulder portion, valve 20106 may be secured to the floor of device body 20101 by an adhesive, by spot welding, or by mechanical clamping, for example. Other geometric configurations of the valve and other mounting techniques are also possible. The foregoing discussion of inlet valve 20106 is also applicable, at least in some embodiments, to outlet valve 20108.

The positive and negative leads 20109 are connected to control circuit 20110. The control circuit 20110 includes a power supply 20111 (e.g., battery) or other type of charge storage device (e.g., capacitance). In one example implementation, the control circuit 20110 has a switch 20112 which is selectively closed to provide voltage to the inlet valve 20106, and a switch 20113 which is selectively closed to provide voltage to the outlet valve 20108.

FIG. 20T shows inlet valve 20106 being flexed in response to application of non-zero voltage to the piezoelectric element of inlet valve 20106 for permitting fluid to enter into pumping chamber 20103. In FIG. 20T the outlet valve 20108 remains unflexed for covering outlet port 20107. In another embodiment inlet valve 20106 may remain unflexed for covering inlet port 20105 while outlet valve 20108, in response to application of voltage to the piezoelectric element of outlet valve 20108, permits expulsion of fluid from pumping chamber 20103 through outlet port 20107. In another embodiment, both the inlet valve 20106 and the outlet valve 20108 are open and a pump may be eternal and connected to inlet valve 20106 and the outlet valve 20108 with the chamber 20103 not necessarily being a pumping chamber but acting as a passage between inlet valve 20106 and outlet valve 20108 as shown in FIG. 20U and FIG. 20V.

According to an embodiment, control circuit 20110 includes a timer 20114 which times or controls the duration of opening and/or closure of switch 20112 and/or switch 20113, and thus the opening and closing of inlet valve 20106 and/or outlet valve 20108. The timer 20114 can take any suitable form, from a simple circuit or delay line to a microprocessor, and is operated, sequenced, or programmed in accordance with a desired operation of the device 20100, e.g., to match the desired frequency of operation of device 20100. In an embodiment, the pump operation, valve controls, may be external. The device/system corresponds to the active micro valve as shown in FIG. 20U and FIG. 20V. In one mode of operation, the timer 20114 likely operates the two switches 20112 and 20113 with differing signals and thus differing timings, so that the inlet valve 20106 and outlet valve 20108 are not necessarily open at the same time.

Those skilled in the art will appreciate that the active outlet valve 20108 of the embodiment can be opened or activated by appropriate signal or voltage as discussed in conjunction with active inlet valve 20106. Moreover, the opening and controlling of switches 20112, 20113 can be accomplished via any suitable means, such as (for example) solenoids, Hall Effect devices, relays, or transistors, bearing in mind that switches do not necessarily need to be mechanical but can be partially or entirely electrical.

In an embodiment, one valve may be an active valve and another valve may be a passive valve. In the particular example, the inlet valve may be an active inlet valve, but the outlet valve may be a passive outlet valve. In another implementation, the outlet valve may be active and the inlet valve may be passive.

In one mode of operation, the active inlet valve may be activated to open inlet port while passive outlet valve remains closed. Then, after the pump has been self-primed by sufficient admission of fluid through inlet port, the active inlet valve is kept open (in view of the self-priming) and the passive outlet valve operates (e.g., opens and closes) in accordance with its activation.

As one example way of implementing device of any of the foregoing example embodiments, the actuator 20104 can be a diaphragm and/or include a piezoelectric layer, with the piezoelectric layer causing the displacement of diaphragm 20104 when an electric field is applied to the piezoelectric layer. The electric field is supplied to the piezoelectric layer of diaphragm 20104 by a power supply such as power supply 20115.

Most of the structural features of the device are described above merely to provide an example context for explaining how active valves operate. As such, no particular emphasis or criticality should be assigned to any of the structural elements or position of elements of device 20110. For example, the structure and positioning of the inlet valves and outlet valves are not necessarily germane. The person skilled in the art will appreciate that one or more of the inlet valve and outlet valve can be oriented so that the direction of fluid flow through the valve(s) is parallel to the displacement direction arrow 20116 (e.g., one or more of inlet valve and outlet valve are formed in a bottom wall of device body base 20101).

Alternatively, one or more of the inlet valve and the outlet valve can be oriented so that the direction of fluid flow through the valve(s) is parallel to the displacement direction arrow 20116 (e.g., one or more of inlet valve and outlet valve is formed in a sidewall of device body base 20101) as shown in FIG. 20V.

Moreover, the shape, size, or other configuration of the device body and its device body base 20101 and device body lid 20102 are variable. Variously shaped device bodies, with or without myriad auxiliary or surface features, could be utilized.

Examples of diaphragm type structures which include a piezoelectric layer, and methods of fabricating the such diaphragms and pumps incorporating the same, as well as various example pump configurations with which the present invention is compatible, are illustrated in the following (all of which are incorporated herein by reference in their entirety): PCT Patent Application PCT/US01/28947, filed 14 Sep. 2001; U.S. patent application Ser. No. 10/380,547, filed Mar. 17, 2003, titled “Piezoelectric Actuator and Pump Using Same” U.S. patent application Ser. No. 10/380,589, filed Mar. 17, 2003, titled “Piezoelectric Actuator and Pump Using Same”.

It will be further appreciated that it is possible to control the voltage amplitude applied to the active valves described herein for controlling an opening distance by which the valve displaces relative to the respective port. Thus, the degree of opening effected by the valve is controllable or adjustable, and thus also the flow of fluid through the valve and the pump is adjustable and controllable.

### 9. Phase Change Microvalve:

Referring to FIG. 20W, FIG. 20X, and FIG. 20Y, the present invention provides a preferred embodiment of a phase change microvalve device, which includes a fluid microchannel 20134 and a heat/cold conducting channel 20133, wherein a working medium flows in the fluid microchannel 20134, and the on-off of the fluid microchannel 20134 needs to be controlled to adjust the flow state of the working medium, and the heat/cold conducting channel 20133 conducts heat or cold to adjust the phase state of the working medium in the fluid microchannel 20134, thereby realizing the on-off adjustment of the fluid microchannel 20134.

The first end of the heat/cold conducting flow channel 20133 is attached to the fluid microchannel 20134, and the second end of the heat/cold conducting flow channel 20133 is connected with the heat source system and/or the cold source system. The heat source system and the cold source system transmit heat or cold to the fluid micro-channel 20134 through the heat/cold conducting channel 20133. When the cold source system transmits the cold to the fluid micro-channel 20134, the working medium in the fluid micro-channel 20134 is cooled until the phase state of the working medium is changed to the preferred phase state, for example, the working medium is cooled and changed into a solid phase, hence, the flow of the working medium in the fluid micro-channel 20134 is blocked. When the working medium needs to start flowing in the fluid micro-channel 20134, the cold source system finishes transmitting cold energy to the fluid micro-channel 20134, the heat source system transmits heat to the fluid micro-channel 20134 through the heat/cold conducting channel 20133, so that the phase state of the working medium in the fluid micro-channel 20134 is changed to conduct flow in the fluid micro-channel 20134, the working medium absorbs the heat, the phase state of the working medium is changed from a solid phase to a liquid phase or a gas phase, and the working medium returns to the flowing state.

The fluid micro-channel 20134 and the heat/cold conducting channel 20133, both integrated on the micro-fluidic chip, are coplanar. The heat/cold conducting channel 20133 is provided with the side surface of the fluid micro-channel 20134, and the heat/cold conducting channel 20133 is attached to the side surface of the fluid micro-channel 20134. The second end of the heat/cold conducting channel 20133 may also be provided as an inlet for a heat conductor, which is poured into the heat/cold conducting flow channel 20133 through the second end of the heat/cold conducting flow channel 20133.

Referring to FIG. 20W, a microporous structure 20132 is disposed on an end surface of the heat conducting channel 20133 and the fluid microchannel 20134, a heat/cold conducting channel 20133 transmits heat or cold to a working medium in the fluid microchannel 20134 through the microporous structure 20132, the heat/cold conducting channel and the working medium directly contact with each other through the microporous structure 20132 to transmit the heat or cold, but the microporous structure 20132 can prevent the heat/cold conducting channel from flowing into the fluid microchannel 20134 and the working medium from flowing into the heat/cold conducing channel 2013, the microporous structure 20132 not only has a function of transmitting the heat or cold, but also can separate the heat/cold conducting channel and the working medium, preventing the flow in the heat/cold conducting channel from mixing with the working medium, ensuring the independence and cleanliness of the heat/cold conducting channel and the working medium.

The area in the fluid microchannel 20134, corresponding to the end face, where the heat/cold conducting channel 20133 and the fluid microchannel 20134 are attached to each other, forms a phase change area 20135, the range of the phase change area 20135 can be expanded or reduced in the length direction of the fluid microchannel 20134 by the position of the end face where the heat/cold conducting channel 20133 and the fluid microchannel 20134 are attached to each other, and the range of the phase change area 20135 is related to the heat source system and the cold source system providing heat and cold to the heat/cold conducting channel 20133.

The heat source fluid or the cold source fluid can rapidly change the temperature of the working medium in the phase change region 20135 around the microporous structure 20132, so that the phase change of the working medium can caused the fluid micro-channel 20134 to be opened or closed.

The cold source system or the heat source system transmits cold or heat to the heat/cold conducting channel 20133, the heat/cold conducting channel 20133 transmits the cold or heat to the working medium in the fluid micro-channel 20134, the heat source or the cold source in the heat/cold conducting channel 20133 directly transmits the cold or heat to the working medium through the microporous structure 20132, the heat/cold conducting channel and the working medium are kept separated, and the working medium is prevented from being polluted by fluid mixing.

The heat/cold conducting flow channel 20133 is filled with heat by a heat/cold conductor, the heat/cold conductor plays a role in cold or heat transmission. The heat/cold conductor is in direct contact with a cold source fluid of a cold source system and a heat source fluid of a heat source system in the accommodating structure 20131, the heat/cold conductor is in direct contact with the cold source fluid and the heat source fluid, heat transmission efficiency is improved, and the fluid micro flow channel 20134 is adjusted by being opened and closed quickly. The inlet of the heat/cold conducting channel 20133 is an open structure, and the cold source fluid or the heat source fluid in the containing structure 20131 directly contacts with the heat/cold conductor through the inlet of the heat/cold conducting flow channel 20133.

Specifically, the heat/cold conducting flow channel 20133 is filled with a low-melting-point metal material, the heat/cold conductor comprises the low-melting-point metal material, the low-melting-point metal material belongs to a good heat/cold conductor, the heat or cold conducting effect is good, the performance is stable, and the microporous structure 20132 cannot be penetrated due to large molecular tension. Preferably, the low melting point metal material includes a metal simple substance and a metal alloy, and preferably metallic bismuth or a bismuth alloy is used.

The material of the micro-fluidic chip belongs to a poor heat/cold conductor, Polydimethylsiloxane (PDMS), glass or quartz is preferably adopted to prevent heat loss, and the low-melting-point metal material has better heat-conducting properties than the material of the micro-fluidic chip, so that heat conduction is ensured.

After the low-melting-point metal material is heated by the heat source fluid to be liquid, the microporous structure 20132 can prevent the liquid low-melting-point metal material from entering the fluid microchannel under the action of surface tension. Meanwhile, the working medium (solid or liquid) is blocked by the low-melting-point metal material and cannot enter the heat/cold conducting flow channel 20133. The temperature in the heat/cold conducting flow channel 20133 is reduced when the temperature is restored to the solidification point of the low-melting-point metal material, the low-melting-point metal material is cooled and solidified into a solid in the heat/cold conducting flow channel 20133. Generally, the low-melting-point metal material is in a solid state at room temperature, and therefore, when the working medium flows through the fluid micro flow channel 20134 under the driving of pressure at room temperature, the low-melting-point metal material does not flow out of the heat/cold conducting flow channel 20133, and the low-melting-point metal material is not washed away.

At room temperature, a small amount of solid low-melting-point metal material can be left to be directly contacted with the cold source fluid or the heat source fluid. The micro-fluidic chip material belongs to a poor conductor of heat, and the low-melting-point metal material belongs to a good conductor of heat. Therefore, after the cold source fluid or the heat source fluid contacts the low-melting-point metal material, the heat can be rapidly transferred to the fluid in the phase change region 20135 through the low-melting-point metal material without being diffused to the microfluidic chip material around the heat/cold conducting channel 20133. The size of the range of the phase change region 20135 can be varied by controlling the amount of cold provided by the cold source fluid. Therefore, the phase change micro-valve device has the advantages of simple operation, good controllability, wide compatibility and the like.

The cold source fluid of the cold source system comprises low-temperature liquid fluid, the temperature range of the low-temperature liquid fluid is between −200° C. and −100° C., and the cold source fluid preferably adopts liquid nitrogen, or liquid oxygen, or liquid argon, liquid air and the like; the heat source fluid comprises liquid or gaseous high-temperature fluid, the temperature of the high-temperature fluid is in the range of 40-60° C., and hot air and hot water are preferably selected.

At least one containing structure 20131 is connected to a second end of the heat/cold conducting flow channel 20133, and the containing structure 20131 is used for containing a cold source fluid and/or a hot source fluid.

The second end of the heat/cold conducting flow channel 20133 can be connected with an accommodating structure 20131, the cold source system and the cold source fluid in the cold source system and the heat source fluid in the heat source system share one accommodating structure 20131, and when the fluid micro flow channel 20134 needs to be closed, the cold source system supplies the cold source fluid to the accommodating structure 20131 until the working medium in the fluid micro flow channel 20134 is converted into a solid phase, so that the flow of the working medium is blocked; when the fluid micro-channel 20134 needs to be opened, the cold source fluid of the cold source system is led out of the accommodating structure 20131, the heat source system provides the heat source fluid into the accommodating structure 20131, the working medium absorbs the heat of the heat source fluid to change into a fluid state, the fluid micro-channel 20134 is opened, and the working medium flows stably.

In addition, the second end of the heat/cold conducting flow channel 20133 can be further connected with two accommodating structures 20131, cold source fluid of the cold source system and heat source fluid of the heat source system are respectively introduced into one accommodating structure 20131, when the fluid micro flow channel 20134 needs to be closed, the cold source system introduces the cold source fluid into the accommodating structure 20131 communicated with the cold source system, when the fluid micro flow channel 20134 needs to be opened, the cold source fluid in the cold source system is led out of the accommodating structure 20131, and the heat source system introduces the heat source fluid into the accommodating structure 20131 communicated with the heat source system.

Furthermore, the second end of the heat/cold conducting flow channel 20133 may be further connected to a plurality of accommodating structures 20131, the cold source system and the heat source system may share the plurality of accommodating structures 20131, when the fluid micro flow channel 20134 needs to be closed, the cold source fluid is introduced into all the accommodating structures 20131, and when the fluid micro flow channel 20134 needs to be opened, the cold source fluid is guided out of the accommodating structures 20131 and introduced into the heat source fluid in all the accommodating structures 20131. A plurality of containment structures 20131 may also be distributed to the cold source system and the heat source system as required, for example, the containment structure 20131 includes five structures, two of which are communicated with the cold source system and three of which are communicated with the heat source system.

The receiving structure 20131 may be configured as a receiving groove, a receiving pipe, a receiving ball, etc.

Further, the microporous structures 20132 are uniformly distributed on the end faces, which are attached to the heat conduction flow channel 20133 and the fluid micro flow channel 20134, and the contact area between the heat conductor and the working medium is increased by uniformly distributing the microporous structures 20132.

The microporous structure 20132 is preferably a microwell or an array of microwells. The shape of the micropores is preferably circular, rectangular, or trapezoidal. The distance between the micropores is preferably between 5 and 20 microns.

The processing method of the microporous structure 20132 comprises photoetching processing, and a boss is formed between every two adjacent microporous structures 20132. The processing method of the microporous structure 20132 may be any processing method capable of processing the microporous structure 20132 having an appropriate size, such as laser processing.

In the phase-change micro-valve device, a heat/cold conducting flow channel 20133 and a fluid micro-flow channel 20134 are integrated on a micro-fluidic chip and synchronously manufactured by adopting a micro-processing manufacturing process. Preferably, the micromachining process uses a conventional soft etching technique and uses the same mask to etch the heat/cold conducting channel 20133 and the fluid micro-channel 20134 with equal height and coplanarity on the microfluidic chip material, and the microporous structure 20132 at the intersection of the heat/cold conducting channel 20133 and the fluid micro-channel 20134 can also be synchronously manufactured. Specifically, a hole puncher is used for manufacturing a micropore at the second end of the heat/cold conducting flow channel 20133 to serve as a cold source fluid groove or a heat source fluid containing structure 20131, the diameter of the containing structure 20131 is preferably 5-2 micrometres, and the containing structure 20131 and the heat/cold conducting flow channel 20133 are equal in height. Preferably, the microfluidic chip uses PDMS as a base material. Therefore, the phase change micro valve with micron scale size can be manufactured by the micro-processing manufacturing process, and the manufacturing is simple and the cost is low. And the phase change micro valve device is very convenient to integrate a plurality of phase change micro valve devices or integrate the phase change micro valve devices and other components in a microfluidic system.

In some embodiments, as shown in FIG. 20W, the fluidic micro-channel 20134 has an inlet and an outlet, and the heat/cold conducting channel 20133 is disposed on only one side of the fluidic micro-channel 20134. The working principle of the phase change micro-valve device is as follows: when the fluid micro-channel 20134 needs to be closed, the accommodating structure 20131 connected with the second end of the heat/cold conducting channel 20133 is an accommodating groove for accommodating cold source fluid, a proper amount of cold source fluid is regularly put into the accommodating groove, the temperature of the low-melting-point metal material in the heat/col conducting channel 20133 is rapidly reduced, the liquid working medium in the phase change region 20135 is cooled and solidified into a solid, the solid state can be maintained for a long time, and the phase change is controlled to only occur in the phase change region 20135. The size of the phase change region 20135 changes with the amount of the source fluid placed in the holding tank; when the fluid micro-channel 20134 needs to be opened, the accommodating structure 20131 connected with the second end of the heat/cold conducting channel 20133 is an accommodating tank for accommodating heat source fluid, and after the heat source fluid is placed into the accommodating tank, the temperature of the low-melting-point metal material in the heat/cold conducting channel 20133 is rapidly increased, so that the solid working medium in the phase-change region 20135 is heated and melted into liquid, and the conduction of the fluid micro-channel 20134 is realized.

In some technical solutions, as shown in FIG. 20X, two heat/cold conducting flow channels 20133 are symmetrically arranged on two sides of a fluid microchannel 20134, each heat/cold conducting flow channel 20133 is connected with a cold source system and a heat source system, and a microporous structure 20132 is arranged at a connection part of each heat/cold conducting flow channel 20133 and the fluid microchannel 20134. Each heat/cold conducting flow channel 20133 is filled with a low melting point metal material and has an inlet.

The fluidic microchannel 20134 comprises one inlet, one outlet, or one inlet, a plurality of outlets, or a plurality of inlets, one outlet, or a plurality of inlets, a plurality of outlets. When the number of the inlets or outlets of the fluid micro-channels 20134 is more than one, the fluid micro-channels 20134 comprise fluid micro-streams, one inlet and one outlet which are communicated with each other form one fluid micro-stream, and each fluid micro-stream is symmetrically provided with two heat/cold conducting channels 20133.

The working principle of the phase change micro-valve device is as follows: when the fluid micro-channel 20134 needs to be closed, the cold source system fills the cold source fluid into the containing structure 20131, and a proper amount of cold source fluid is regularly put into the containing structure 20131 connected with the two heat/cold conducting channels 20133 at the same time, so that the temperature of the low-melting-point metal material is rapidly reduced, the liquid working medium in the phase change region 20135 is cooled and solidified into a solid, the solid state can be maintained for a long time, and the phase change is controlled to only occur in the phase change region 20135. When the fluid micro-channel 20134 needs to be opened, the heat source system fills the heat source fluid into the containing structure 20131, the heat source fluid is simultaneously put into the containing structures 20131 of the two heat/cold conducting channels 20133, the temperature of the low-melting-point metal material is rapidly increased, and then the solid working medium in the phase change region 20135 is heated and melted into liquid.

Compared with the mode that the heat/cold conducting flow channel 20133 is arranged on one side of the fluid micro-flow channel 20134, the heat/cold conducting flow channel 20133 is additionally arranged on the other side of the fluid micro-flow channel 20134, so that the response speed of the phase change micro-valve device can be increased, and the working stability of the phase change micro-valve device can be improved.

In some technical solutions, as shown in FIG. 20Y, one accommodating structure 20131 may provide a cold source fluid and/or a heat source fluid to a plurality of heat/cold conducting flow channels 20133 at the same time, one accommodating structure 20131 is connected to second ends of the plurality of heat/cold conducting flow channels 20133, the plurality of heat/cold conducting flow channels 20133 are integrated on one accommodating structure 20131, a first end of each heat/cold conducting flow channel is connected to a fluid microchannel 20134, and each guiding flow channel provides cold or heat to the fluid microchannel 20134 connected thereto.

Preferably, the heat/cold conducting flow channels 20133 are connected to the side wall of the containing structure 20131 and distributed radially to the periphery of the containing structure 20131, and each heat/cold conducting flow channel 20133 can be connected with one fluid micro-channel 20134 and simultaneously adjusts a plurality of fluid micro-channels 20134, so that the efficiency is improved. In addition, all the heat/cold conducting flow channels 20133 can also be connected to an annular or special-shaped fluid micro-flow channel 20134, and meanwhile, the opening and closing of different parts on one fluid micro-flow channel 20134 are adjusted, so that the connection and disconnection of the fluid micro-flow channel 20134 and other flow channels are adjusted.

In addition, each heat/cold conducting flow channel 20133 can be connected with an additional accommodating structure for supplementing cold or heat. The heat/cold conducting flow channel 20133 is additionally connected with an accommodating structure which is suitable for fluid micro-flow channels 20134 or fluid micro-tributaries with different cold or heat requirements when being used for supplementing cold or heat.

Specifically, there are 20136 fluidic channels 20134, and each fluidic channel 20134 has an inlet and an outlet independently. The number of the heat/cold conducting flow channels 20133 is equal to that of the fluid micro-flow channels 20134, 20136 that are provided, each heat/cold conducting flow channel 20133 is only crossed with one fluid micro-flow channel 20134, and the micropore structure 20132 is arranged at the crossed position. The heat/cold conducting flow channels 20133 are filled with a low-melting-point metal material and share a receiving structure 20131. The number of the phase change regions 20135 is equal to that of the fluid micro-channels 20134, and 20136 that are provided. The number of the fluid microchannels 20134 is not limited to 20136, and may be set according to actual needs.

The working principle of the phase change micro-valve device is as follows: the shared accommodating structure 20131 is connected with a cold source system and/or a heat source system, the heat/cold conducting flow channels 20133 are connected on the accommodating structure 20131, the accommodating structure 20131 simultaneously provides cold or heat for all the heat/cold conducting flow channels 20133, and simultaneously controls the opening and closing of the fluid micro-channels 20134 and the fluid micro-branches; when a plurality of fluid microchannels 20134 or a plurality of fluid tributaries in the fluid microchannels 20134 need to be closed, the accommodating structure 20131 is connected with a cold source system, the cold source system regularly puts a proper amount of cold source fluid into the accommodating structure 20131, the temperature of working media in all the fluid microchannels 20134 or all the fluid tributaries is rapidly reduced, the liquid working media in the phase change regions 20135 of the fluid microchannels 20134 or the fluid tributaries are cooled and solidified into solids, the solids can be kept solid for a long time, and the phase change is controlled to only occur in the phase change regions 20135. When the fluid micro-channel 20134 or the fluid micro-branch needs to be opened, the containing structure 20131 is connected with a heat source system, the heat source system regularly puts a proper amount of heat source fluid into the containing structure 20131, the temperature of the working medium on the fluid micro-branch or the fluid micro-channel 20134 is rapidly raised, and then the solid working medium in the phase change region 20135 is heated and melted into liquid.

The technical scheme shows that all parts can be easily integrated together, the heat/cold conducting flow channels 20133 share one containing structure 20131, the opening and closing states of the fluid micro-flow channels 20134 can be controlled simultaneously, and the integration level of the micro-flow control chip is improved compared with the technical scheme that one heat/cold conducting flow channel 20133 is connected with one independent containing structure 20131.

### 10. Pneumatic Micro Valve

FIG. 20Z shows embodiments of the invention of an ejector operating according to a valve principle and FIG. 20AA shows embodiments according to the invention operating as a fluid displacement ejector.

In an embodiment, a fluid ejector is working according to the valve principle. The valve principle is basically a continuous working principle, which can be adopted to produce stationary fluid jets. A drop on demand operation is achieved by using very short valve-open times (in the order of microseconds to milliseconds), while variable dispense volumes are achieved by using variable valve-open times.

FIG. 20Z, A shows a diaphragm valve as an embodiment of a fluid ejector. An actuation element in the form of a diaphragm 20140, which is pressurized on one side by the control pressure pc, gets into contact with a sealing surface 20141, when the control pressure pc exceeds the fluid pressure pFl, thus a valve opening 20142 is closed, which is connected to the fluid outlet 20143 (FIG. 20Z, A, left). On the other hand, if the control pressure pc is smaller than the fluid pressure, the fluid pressure lifts the diaphragm 20140 from the sealing surface 20141 and fluid flows through the valve opening 20142 and discharges from ejector at the fluid outlet 20143. A diaphragm valve offers a high leak tightness due to the flexibility of the diaphragm, high speed operation due to the very small mass of the diaphragm and the advantage of easy fabrication. Within this invention the use of the term “diaphragm” shall not be limited to the narrow definition as used in the science of the strength of materials, whereupon a diaphragm only is able to transfer tensile forces. Instead, within this document the term shall also be extended to the case of the “plate”, which is able to transmit bending moments, which means, that a diaphragm can be made of a more solid material or can have thicknesses, which usually fit into the definition of a “plate”. Regarding the material of the diaphragm there also shall be no restrictions within this invention, suitable materials are for example, metals, thin glass, silicon, SiN, thermoplastics (such as PTFE, E/TFE, PFA, PVC, ABS, SAN, PP, PA, POM, PPO, PSU, PEBA, PEEK, PEI, Designations according to ISO 1043. 1), thermoplastic elastomers (TPE), elastomers (such as NBR, HNBR, CR, XNBR, ACM, AEM, MO, VMQ, PVMQ, PMQ, FVMQ, FKM, FFKM, AU, EU, ECO, CSM, NR, IR, BR, SBR, EPDM, EPM, IIR, CIIR, BIIR, TPE, description according to ISO 1629), polyimides, rubber and vulcanisate, natural-/synthetical rubber, thermosets (such as UP, PF, UF, UP-GF, description according to ISO 1043. 1), all polymers, including filled or fiber-reinforced polymers.

In a variant of the printhead according to the invention, the ejectors are double diaphragm valves, actuated by the control pressure pc. FIG. 20Z, B shows a double-diaphragm valve as a further embodiment of a fluid ejector. Instead of using a single diaphragm 20140 as shown in FIG. 20Z, A, which combines the functions of valve actuation and sealing, these functions are realized here separately. A first diaphragm 20140 with an area A1 is subjected to a static pressure, which is transmitted via a coupling element 20144 onto a sealing element, which seals the valve. The fluid contacts a second diaphragm 20140, having an area A2 and being connected to a coupling element and a sealing element 20145. The coupling element may be realized loosely as an insert, for example, as an inserted sphere or cylinder, or may be realized as a compound together with the first diaphragm and/or the second diaphragm 20140. As well as the first diaphragm, coupling element, second diaphragm 20140 and sealing element, there can be a single coherent structure, for example, made of an elastomer material. With AZ<A1 sufficient axial force and surface pressure for sealing the valve opening can already be achieved with control pressures pc1<pFl. The double-diaphragm valve, as depicted as an example, has two ports for control-pressures, which can be fed in different combinations, whereupon it makes sense, however, to keep one of the two control pressures pc1 and pc2 constant and to actuate the other by means of the micro-electro-pneumatic circuit.

For example, pc1 is fed by a static pressure pst=pFl, while the control pressure of the micro-electro-pneumatic circuit is connected to pc2. When pc2 increases, the fluid valve opens as a result of the effective fluid pressure pFl even before the pressure pc2 reaches the pressure level pFl of the fluid. Opposite, Pc1 also can be fed with the control pressure provided by the micro-pneumatic circuit, while pc2 is held constant at atmospheric pressure, for example.

FIG. 20Z, C shows a single diaphragm valve comprising a mechanical coupling and sealing device as another embodiment of a diaphragm-actuated fluid ejector. A rod 20146, sealed by a radial sealing, transfers the motion of the diaphragm to a valve seal 20145. Actuation is done using the ports pc1 and pc2 by applying a suitable combination of a static pressure and a control pressure. In the embodiment, the fluid inlet is shown as 20147.

Further embodiments of a pneumatically actuated fluid valve, that are not presented here, can consist of all kinds and forms of mechanical actuation elements, seal elements or translation mechanics such as tipping and leveraging elements, pneumatically deformable bellows, tubes or balloons or pneumatically actuated pistons to achieve a closing and opening effect of the valve opening 20142.

To explain the devices and methods according to the invention in FIG. 20AA, there are different exemplary embodiments of pneumatically actuated fluid ejectors illustrated, which work according to the fluid displacement principle. These are preferably used in free jet dispensing applications. The embodiments are each illustrated for the states of suction and ejection, which are the characterizing states of the fluid displacement principle.

FIG. 20AA, A illustrates the basic working principle of a pneumatically actuated fluid displacer for generating a fluid ejection. Fluid shall be available at the fluid inlet 20150 with a pressure pFl, which is in the magnitude of the ambient pressure pu. During suction phase, the control pressure pc is lower than the pressure of the fluid pfl. In the case of an ideal flexible diaphragm 20151, the control pressure is transferred lossless to the fluid located in the ejector-cavity 20152, so that the ball valve 20153, for example, whose ball is held in position by a valve spring 20154, opens to let the fluid enter the ejector-cavity. By switching the control pressure to pc>pFl, the inlet valve 20156 changes into the closed state, while the fluid outlet valve 20157 opens and fluid is expelled. To obtain a clean fluid output and a clean fluid tear-off, a fast flap of the control pressure between its two states is necessary, which according to the invention is achieved by use of a micro-electro-pneumatic circuit.

Fluid discharge (FIG. 20AA, B) is done according to the invention by a highly transient pressure pulse of control pressure pc. This transmits through the diaphragm 20151 to pressurize the fluid in the ejector cavity 20152. The fluid pressure is then released through an ejection of fluid through the fluid outlet 20158. The pressure pulse of the control pressure is followed by a rapid drop of the control pressure to its lower pressure level, which in the best case is below the fluid pressure pFl (embodiment in FIG. 20AA, B). With the drop of the pressure inside ejector cavity below pFl, fluid is delivered through the inlet opening into the ejector cavity 20152. The under pressure inside ejector cavity 20152 simultaneously acts on the fluid outlet. Only the capillary forces of the fluid meniscus in the orifice of the fluid outlet 20158 allow the buildup of a vacuum inside the ejector cavity and prevent that air is sucked in through the fluid outlet during this phase.

In FIG. 20Z, C, as an example, the following case is illustrated, that a stiffer diaphragm 20140 is used having higher restoring force than the one in the case of FIG. 20AA, B. In this case, the restoring force of the diaphragm is predominantly used to suck the fluid in the ejector cavity 20152 during the suction phase. The lower control pressure level therefore does not need to be as under pressured as in the example of FIG. 20AA B.

Ejectors, according to the invention, working according to the fluid displacement principle, can be actuated advantageously by a configuration of the micro-electro-pneumatic circuit. Using this configuration, due to the fast opening of the normally closed piezoelectric valve, as a result of the electrical control signal, fast control pressure changes in the microsecond range can be realized, which favors a fluid discharge. During the suction phase a slow pressure drop inside the ejector cavity 20152 is desirable to ensure that the pressure does not fall below the capillary pressure in the fluid outlet and, as a consequence, air is sucked through the fluid outlet 20158 into the ejector cavity instead of fluid through the fluid supply 20150. A slow drop of the pressure in the ejector cavity is achieved by designing the micro-electro-pneumatic circuit such that the time constant for the decrease of control pressure is higher than the time constant for the increase of the control pressure.

In FIG. 20AA, D two further advantageous developments of FIG. 20AA, B are outlined, which can also be realized individually. First, the diaphragm 20151 has two positive-fitting stops, so that the expelled fluid volume of the ejector cavity is exactly defined by the geometry. Second the diaphragm 20151 when getting into contact with the lower surface closes the fluid outlet 20158, thus enforces an abrupt tear off of the fluid at the fluid outlet.

It should be noted that the invention is not limited to the illustrated embodiments, which only constitute examples of possible embodiments, because it is not essential in the present invention which nature of the ejector and in particular the nature of the operating element of the ejector is, as long as its structural design throughout exhibits the required pneumatic controlled valve effect with sufficiently small response time and sufficiently high fluid throughput.

### 11. Pinch-Point Valve

FIG. 20BB, A-C provide a schematic diagram illustrating various stages of a micro-fluidic channeling device according to an embodiment of the present invention.

Referring now to A-C of FIG. 20BB, an elastically resilient or memory material can be utilized in forming a micro-fluidic channeling device 20160. The micro-fluidic channeling device can be formed in a cylindrical tube or conduit shape having an initial open configuration (FIG. 20BB A), a heat- and pressure-induced closed configuration (FIG. 20BB, B), and a heat-induced re-opened configuration (FIG. 20BB, C). The micro-fluidic channeling device can be formed, in cross-section perpendicular to the direction of fluid flow 20161, as a cylindrical or circular shape, a triangular shape, an oval shape, or a square or rectangular tubular shape, for example. In this embodiment, the open or closed configuration is determined by the distance between the protrusions, nodes or bulges 20164 that can be present to create the elastically resilient pinch-point 20165. When a micro-fluidic channeling device is in an open configuration, the elastically resilient pinch-point defines a space or gap such that fluid flow is from relatively to completely unimpeded and continuous. If a micro-fluidic channeling device is in a closed configuration, then the nodes are in a more proximate position with respect to each other, thereby forming a pinch-point. The pinch-point acts to reduce, restrict, or completely stop the fluid flow.

To form the pinch-point 20165 shown in FIG. 20BB, B, application of heat 20163 and optionally pressure 20162, to a discrete location at or around the location of the nodes can provide acceptable results. Because of the nodes 20164, pressure may not be required to form the pinch-point, as the material may swell such that the nodes are brought closer together, though it is usually preferred to apply some pressure. As noted above, once the material achieves and/or surpasses the glass transition temperature, it becomes ductile and pliable. The addition of heat and pressure in the discrete location forms the restrictive pinch-point from the micro-fluidic channeling device wall. Sufficient pressure can be applied through any solid or rigid tool which is capable of deforming the ductile elastomeric material from an open configuration to a more restrictive configuration.

Once the micro-channel has been restricted by the sequential application and withdrawal of heat, and optionally pressure, the channel can be re-opened by the application of additional heat 20163 as shown in FIG. 20BB, C. Additionally, negative external pressure may be applied to a closed pinch-point configuration to ease the reformation process of the micro-fluidic device, or alternatively, the fluid pressure from within the channel can also aid in re-opening the pinch-point upon application of heat.

Thus, in one aspect of the invention, a device that is pre-formed in a closed configuration can be loaded with a liquid. The pinch-point 20165 of the device can thus be poised to elastically open upon application of heat, thereby releasing or allowing fluid flow 20161. It should be noted that when forming the pinch point, in an embodiment, complete melting of the material should not occur. This is because if complete melting of the material occurs, the pinch point will lose its stored energy or elasticity, and thus, will not elastically reopen when subsequent heat is applied. Thus, when forming the pinch point, the heat and pressure should be great enough to create the pinch point but should be less than the amount of heat and/or pressure that would permanently reconfigure the pinch point so that it loses its elasticity. This being stated, as shown in FIG. 20BB, C, upon application of heat 20BB, B, the resilient material becomes reconfigured to restore or substantially restore the pinch point and allow fluid flow 20161. In other words, when heat is applied to the pinch point, the pinch point can be reopened, restoring fluid flow.

In an embodiment, the pinch valve can be made of different configurations, such as tube configuration with the force applied in the direction of fluid flow to cause the tube to bulge and restrict the fluid flow. Similarly, piezo electric, shape memory pneumatic, hydraulic principles can be used to create the pinch at the selected locations either by creating an additional structure or integrating it into the tube portion around the pinch points depending on the location where the shape deformation or pinch is required.

### N. Device Configured for Wireless Communication

Medical implants are used to deliver drugs, collect information about the body and also to interact with the body in a variety of situations. All of these medical implant devices provide an advantage if wireless communication is possible with other devices outside or inside the body.

### 1. Wireless Communication Via Medical Implant Communication System

Medical Implant Communication System (or MICS) is a short-range communication technology which is used for transmitting data to medical devices implanted in the body and receiving data from the medical device. It operates at radio frequency (typically from 402 to 405 MHz) in a short distance range (typically 2 mega transfers per second (mts)).

In an embodiment, the implantable device could be equipped with the microcontroller interfaced with the MICS band transceiver. This configuration can enable the implant to bi-directionally communicate with the Base station outside the human body within a short range. The base station will have a similar transceiver and the necessary built-in intelligence to communicate data.

In some embodiments, the base station could be equipped with communication technologies like Wi-Fi to connect with the rest of the world. This configuration will allow the medical implant to exchange data over the internet (via base station) to medical professionals. The end equipment receiving data could be a Mobile phone, a PC or any other internet enabled device.

In some embodiments, the base station could contain the alternate technology like GSM/GPRS using whichever data could be transferred over the cellular network. In a typical scenario, the data read from the implant using this technology could be the data collected by various sensors in the implant's electronics.

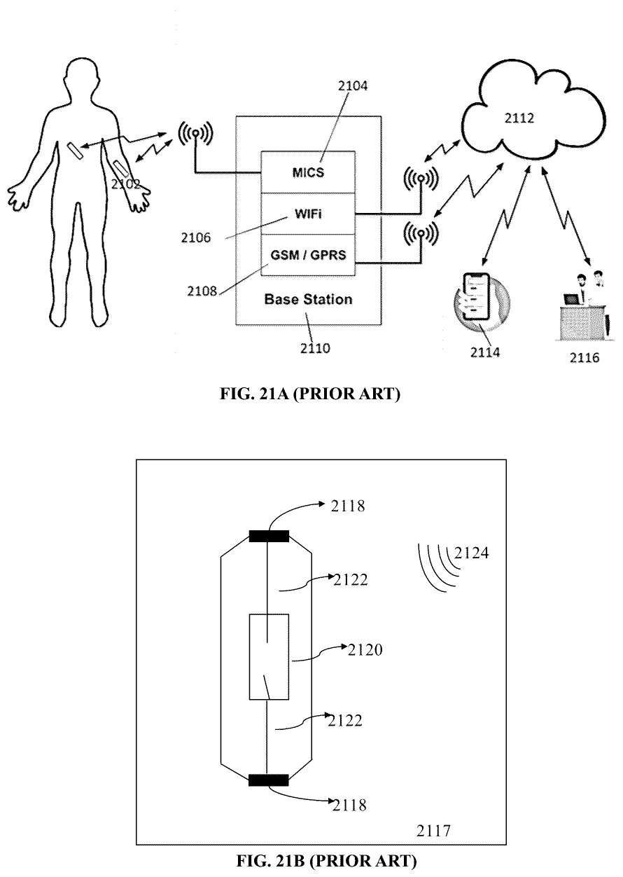

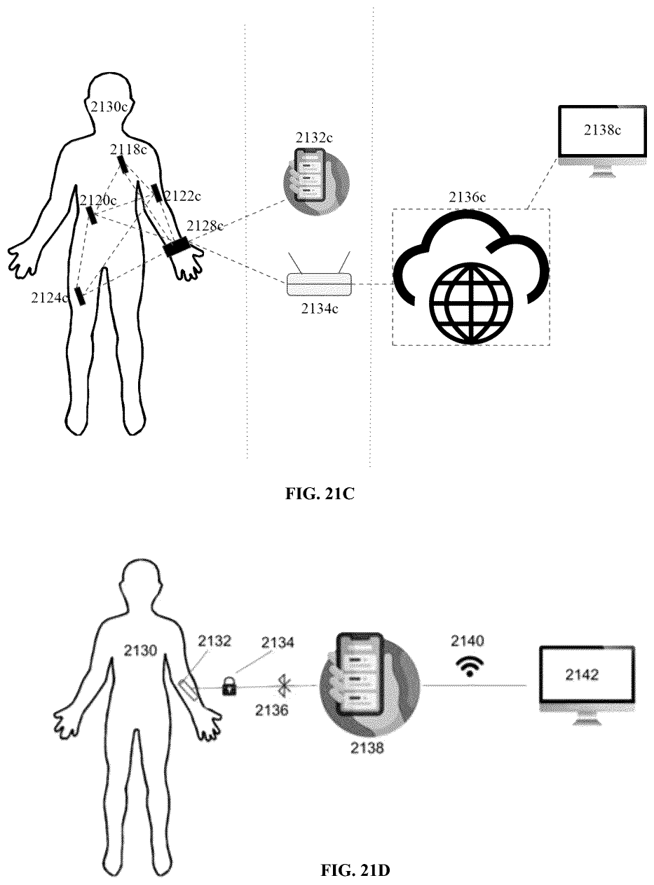

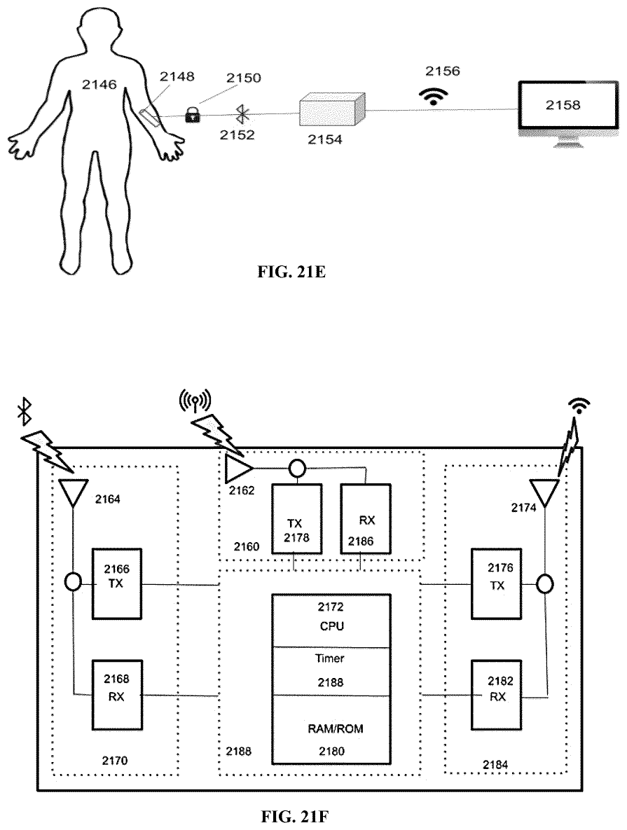

Referring to FIG. 21A, it shows communication between an implantable device and an external device, according to one or more embodiments.

The implanted device 2102 can be equipped with MICS transceiver which will transmit the data over MICS Radio Frequencies to the base station. The communication will involve much more than just the physical layer communication. Communication module has its own data communication protocol, error handling protocol, data security measures etc. The MICS transceiver module 2104 within the base station 2110 communicates with the implanted device 2102 to receive/transmit data. The Wi-Fi module 2106 within the base station 2110 communicates with the outside world over the internet. This module establishes the link between MICS system on one side and the external devices on the internet on the other side. The GSM/GPRS module 2108 within the base station 2110 offers an alternate technology to exchange data over the internet. The base station 2110 on one side communicates with the implanted device over MICS and on the other side, exchanges data with the remote devices like mobile phones or PCs over internet. The base station could be an exclusively designed standalone system based on the microprocessor or microcontroller as the brain, or could be even a PC with the relevant hardware and software installed on it. In an embodiment, an external device for example, a smart phone 2114, near the base station or at a remote location anywhere in the world, can monitor or exchange data with the implant device using a combination of the technologies listed above. In an embodiment, an external device for example, a desktop/laptop 2116, near the base station or at a remote location anywhere in the world, can monitor or exchange data with the implanted device using a combination of the technologies listed above.

### 2. Wireless Communication Via Backscatter

Backscatter is a method that uses an incident radiofrequency (RF) signal to transmit data without a battery or power source. It employs passive reflection and modulation of the incoming RF signal and converts it into tens or hundreds of microwatts of electricity, that can be encoded for data communications.

Implant devices having a wireless communication function and also wireless power transmission have been proposed in the prior art. For example, US 2013/0215979 describes a method for simultaneously transmitting power and data from outside the body to an implant device. Circuitry in the implant device is used to harvest power from the external RF signal and there may be backscatter communication from the implant to the external device. Load modulation is proposed to transmit the data signal back to the external device. Part of the charging RF signal is used for backscatter communication and the rest is used for wireless power transmission.

Referring to FIG. 21B, it shows communication between an implantable device and an external device using backscattering, according to one or more embodiments. The implantable device 2117 comprises a data source; and a non-magnetic resonant antenna for backscatter communication with an external communication system, wherein the non-magnetic resonant antenna comprises at least two electrodes 2118 comprising two conductive patches disposed to be spaced apart when the implant device is in use; the implant device is arranged to control the backscatter characteristics of the non-magnetic resonant antenna to transmit data from the data source to the external communication system; the implant device is arranged so that the backscattering characteristics of the non-magnetic resonant antenna changes the impedance of the non-magnetic resonant antenna by switching between the coupling of at least two electrodes through the body tissue 2124 and the coupling of at least two electrodes through a conductive path 2122. Arranged to be controlled by one or more electrical switch(s) (2120) comprising the electrical switch.

In an embodiment, the antenna can be printed on the surface of the implantable/implanted device. In some embodiments, the antenna is a non-magnetic resonant antenna. In this context, magnetic resonance means that the antenna structure and geometry are designed in a way that provides resonance (as used in the common antenna method). With non-magnetic resonance (e.g., an electrode antenna), the antenna itself has no resonance, but the impedance of the medium around the antenna can provide resonance. Resonance may be unnecessary for the operation of a non-magnetic resonant antenna, instead the operation of the antenna may be based on a change in impedance. In an exemplary embodiment, when a non-magnetic resonant antenna is used in a conductive medium containing biological tissue, a current path may be created between the two electrodes of the antenna in the conductive medium. The current path extends into the biological environment and creates a larger area with a current distribution in the biological medium. The size of this area is much larger than the physical size of the antenna. This results in a large virtual size for the antenna and thus high efficiency is achieved. In the prior art using a self-resonant antenna, the antenna efficiency is limited by the physical size of the antenna geometry, whereas for a non-magnetic resonant antenna as in the first aspect, the antenna efficiency will be increased using a larger virtual size of the antenna.

The backscattering characteristics of the non-magnetic resonant antenna changes the impedance of the non-magnetic resonant antenna by coupling of two electrodes (2118) through a biological medium (e.g., body tissue, 2124) and switching between the coupling of two electrodes through a conductive path (2122). It is controlled by an electronic switch (2120). Thus, in practice, a high impedance is provided due to coupling through the living tissue when the electrical switch (2120) is open, and a low (nominal zero) impedance is provided through the conductive path through the switch when the switch is closed. Large changes in impedance can be identified by receiving antennas placed on the surface of the body or under the skin. High efficiency of reflection is achieved due to the large radar cross section (RCS), the large bandwidth of the device, and the large impedance change. It can produce a large read range at depths of up to 18 cm with low/medium transmitter power. A high data rate of 12 Mbps has been measured and a potential of 70 Mbps is expected to be achieved.

The external communication system may be arranged to transmit electromagnetic waves towards the implant device and to receive backscattered signals from the implant device. This can be done using a single antenna that acts as both a transmitter and a receiver, but preferably the external communication system includes separate transmit and receive antennas. One reason to use multiple antennas is to reduce the coupling between the transmit and receive antennas to avoid receiver saturation. This allows the receiver to operate over its full dynamic range to receive relatively weak backscattered signals. In addition, in the case of multipath propagation, the use of dual static (dual antennas) or multiple statics (multiple antennas) eliminates the possibility of deep signal fading, thus exhibiting better performance than a single static (single antenna) configuration.

### 3. Wireless Communication Among Multiple Implant Devices

An embodiment relates to the communication system where multiple medical implanted devices within the human body or on the body (wearables) need to exchange data among themselves. The implanted devices could be, but not restricted to devices like drug pump, pacemaker, Defibrillator, cardiac event monitor, kidney function monitor, pancreas function monitor etc.

While one to one communication between implanted devices may be feasible using technologies like Bluetooth Low Energy (BLE); one to many or many to many type of communication may require some type of mesh methodology like one provided by Bluetooth Mesh. Nodes in a Bluetooth Mesh network can be accessed directly from a mobile phone or tablet without the need for a gateway.

FIG. 21C depicts a simplistic implementation of a mesh network of multiple implanted devices where the implanted devices can communicate among themselves without needing the external network connection. Each implanted device in the network functions as a network node. The mesh network could consist of nodes with several feature types.

In one embodiment, most of the implanted devices (2120c to 2128c) could be Low power Nodes which spend most of the time sleeping while their friend node is collecting messages on their behalf. The Low Power Node wakes up at a defined intervals and pings its Friend Node to check for any pending messages. During the same wakeup interval, it may also send messages if any to the other nodes. In an emergency situation, it could wake up and switch on its communication module to send the urgent message to the other nodes within the network.

The mesh network may also contain Relay Node(s). The relay feature enables a node to relay messages to the adjacent nodes. The relay feature enables messages to find multiple paths to the destination node. The Relay nodes and Friend nodes need more power as they will have to be active and scanning all the time. In addition to the above types, there could be one or more proxy nodes within the mesh which act as the interface for a smart phone or to any other external Bluetooth low energy supported device.

In one embodiment, the implanted devices 2118c, 2120c, 2122c, 2124c, 2125c and the wearable device 2128c form the Bluetooth mesh network. The network has the capability to enable its node devices to exchange messages among themselves without needing any external network.

In another embodiment, the wearable device 2128c could act as a Friend Node as well as a Proxy Node. As a friend node it could collect messages on behalf of the Low Power Nodes (Implanted devices) and pass them to the respective Low Power Node when it wakes up. As a proxy node it provides interface for a smart phone 2132c. A dedicated App on the smart phone could be used for the provisioning process to add, remove nodes within the mesh network. A smart phone 2132c could also be used for monitoring various sensor parameters received via the proxy.

In another embodiment, an external hardware device 2134c could act as a gateway between the Bluetooth mesh network and the internet. The gateway hardware will be connected with the mesh network through proxy node 2128c. The Gateway device needs to support Bluetooth low energy to be able to establish connection with the proxy 2118c. This arrangement enables the healthcare provider to remotely monitor/control the implanted devices via a monitor device 2138c connected through the internet.

### Remote Monitoring Using Bluetooth Low Energy (BLE) or Similar Technology

Patients getting implantable devices anticipate a more fulfilling life after receiving an implant, with the expectation that there will be little to no restrictions on their daily activities. As a result, it has become essential to grant patients a high level of mobility while their medical condition is being observed and/or treated with the implantable device. The conventional approach of bi-annual or annual check-ups for both the patient and implantable device restricts the frequency of monitoring. Additionally, the patient may feel obliged to remain near the physician's clinic or hospital where check-ups take place, and emergencies may sometimes arise that necessitate the presence of the attending physician in cases of elderly patients.

Recent advancements in technology have enabled the monitoring of implantable devices from almost any location worldwide. This system allows for communication with an implantable device implanted in a mobile patient. The implantable device comprises a telemetry transceiver that can transmit data to an external device, such as a Smartphone or a proprietary Smart Device (hereafter referred as a Smart Link Device) located near the patient. The communicated data is then sent to a remote medical support network.

Implementation and operation of most, if not all, RF communication systems involves a balancing or compromising of certain countervailing considerations. These are associated with such interrelated operational parameters as data transmission rate and transmission range, among numerous others. For example, the adjustment of one operating parameter may permit or require the adjustment of one or more other operating parameters. At the same time, predetermined system performance goals and/or requirements must be met. Moreover, predetermined limitations imposed upon operational parameter adjustment must be adhered to. These conditions result in the trade-off between signal range and signal power.

In the context of implantable devices, developing systems that allow patients to be monitored remotely in the home require critical modular instrument technology as well as communications systems. This technology can help reduce the number of clinic visits and provide more time for the healthcare provider to respond to changes in the patient's condition.

A network compatible Wireless Link for Medical Device Data Management includes an apparatus for establishing a communication link between an implantable device, an external Smart Device (Smartphone or a Smart Link Device) and a remote monitoring station while at the same time, minimizing the device current drain. The minimal current drain is achieved by scheduling periodic interrogation of the data collected by the implantable device for subsequent transmission to the remote location.

Generally, the communication system is implemented to transmit data preferably being initiated by the implantable device to the Smart Device (Smartphone or Smart Link Device). The implant may be equipped with a Bluetooth or RF communication system. The external Smart Device (Smartphone or a Smart Link Device) is preferably similarly equipped with a Bluetooth or RF transceiver. Use of a Smartphone provides the patient mobility while enabling data transmission and interrogation of the implantable device implanted in the patient, while use of a Smart Link Device instead of a Smartphone helps old patients with minimal technology exposure to have the same facility without the need to operate otherwise technically challenging devices like a Smartphone.

In an embodiment, the device settings, modifications, and performance can be viewed using ‘secured’ apps. As used herein, ‘apps’ refers to types of application software designed to run on a mobile device, such as a smartphone or tablet computer. The mobile app could be developed for multiple operating systems like IOS, Android, etc. A system comprising the device and a mobile phone with an app further comprises a cyber security module, a communication module, a server, and a database.

One embodiment implements a method and apparatus for establishing a communications link between an implantable device, a Smartphone (Or Smart Link Device) and a remote monitoring station while, at the same time, minimizing device current drain. Minimal current drain is achieved by scheduling periodic interrogation of the data collected by the implantable device and subsequent retransmission to a remote location. In addition, the implantable device may also transmit unscheduled data when a significant event, such as a critical fault in the drug pump, occurs.

In yet another aspect of the embodiment, a telemetry transceiver within the implantable device is implemented to communicate data to the external world in an encrypted code. The implantable device telemetry transceiver has a range extending outside the patient's body sufficient to reach a patient's Smartphone or the Smart Link Device. The Smartphone/Smart Link Device equipped with wireless technology like Wi-Fi and/or GSM will have the ability to in turn push the data to the remote medical clinic system. Accordingly, the patient may move freely around the home and/or yard and still be in communication with the remote network or medical clinic via this method.

While the smartphone-based solution provides the patient the ability to monitor his/her own data received from the implantable device through a mobile App, the Smart Link Device mentioned here just acts as a link between the implantable device and the remote clinic. The smart device has a transceiver for both the Bluetooth as well as the wireless communication including the Wi-Fi and GSM.

FIG. 21D is also an illustration of the medical device system adapted in the embodiment. The medical device system shown in FIG. 21D includes an implantable device 2132, could be a drug pump for illustration purposes that has been implanted in a patient 2130. Those of ordinary skill in the art, however, will appreciate that the same arrangement could be advantageously practiced with numerous other types of implantable devices. Also depicted in FIG. 21D is a Smartphone, 2138 for non-invasive, wireless communication with implanted device 2132 via Bluetooth/RF communication 2136. The data communicated over this link is encrypted (2134) and hence secure. Associated with the Smartphone is a dedicated proprietary mobile App and a Wi-Fi and/or Cellular/GSM connectivity for facilitating wireless, or cellular communication 2140 between smartphone 2138 and remote network or monitoring clinic system 2142. The mobile App and the related architecture is further detailed in FIG. 21G.

FIG. 21E is an illustration of the medical device system adapted in the embodiment where a specially designed device 2154 (referred here as Smart Link Device) acts as an information relay between the implant 2148 and remote monitoring clinic system 2158. The medical device system shown in FIG. 21E includes an implantable device 2148, could be a drug pump for illustration purposes-that has been implanted in a patient 2146. Those of ordinary skill in the art, however, will appreciate that the same arrangement could be advantageously practiced with numerous other types of implantable devices. Also depicted in FIG. 21E is a Smart Link Device 2154 for non-invasive, wireless communication with implanted device 2148 via Bluetooth/RF communication 2152. The data communicated over this link is encrypted (2150) and hence secure. Associated with the Smart Link Device is a dedicated proprietary software and a Wi-Fi and/or Cellular/GSM connectivity for facilitating wireless, or cellular communication 2156 between Smart Link Device 2154 and remote network or remote monitoring clinic system 2158.

FIG. 21F is a simplistic block diagram of the Smart Link Device hardware Architecture. 2170 is the Bluetooth/RF transceiver which receives data from the implantable device over Bluetooth link 2164. 2184 is the Wi-Fi transceiver with which the sensor data received over 2164 could be relayed by the processor to the remote server. 2160 is the cellular/GSM transceiver with which the sensor data received over 2164 is relayed by the processor to the remote server. The Smart Link device could choose either the Wi-Fi or Cellular/GSM transceiver depending upon the availability of either of the networks.

All the three transceivers could be interfaced with the processing unit 2172 over serial link, e.g., UART, SPI or I2C. 2172 is the main processing unit which could either be a microprocessor along with the necessary peripherals or a microcontroller. The Timer 2188 is instrumental in scheduling various activities like pushing data to the remote server at regular intervals. RAM/ROM 2180 is where the device's program as well as the transient data is stored. The device connects to the remote clinic servers either through the Wi-Fi equipped transceiver 2184 or Cellular/GSM transceiver 2160 depending on the technical configuration of the device as well as availability of the respective network at that point.

FIG. 21G illustrates the high-level architecture of the Mobile Application provided on the patient's smartphone 2192. The mobile application system could consist of:
The User interface (front end) running on the patient's mobile operating system offers presentation of various vital health parameters received over the Bluetooth/RF link 2190 from the implantable device 21108 of the patient 21106.
A lightweight database on the mobile operating system that temporarily stores the transient data before it's sent to the remote Web Server 21100 over the internet link provided by Wi-Fi or cellular network link 2198.
Part of the backend logic running in the background on the mobile Operating System to receive sensor data sent by the implantable device 21108 over the Bluetooth/RF link 2190.
Part of the backend logic running in the background on the mobile operating system that schedules sending the sensor data received from implantable device to the remote Web Server 21100 over the internet link 2198 (Wi-Fi or Cellular).
Part of the backend logic that runs on the Web server 21100 that handles the data storage, data processing and the remaining major portion of the application logic. The backend services provided by the application logic on the Web Server could be accessed by the patient's mobile App and the remote monitoring Clinic system 21102 by invoking various Web APIs 21104 provided by the application logic.
The Database system 2196 could either be situated physically on the Web Server hardware or anywhere else on the internet cloud.

The Mobile App may present to the patient, the health data (2194), received from the implantable device 21108 as a result of data collected by various sensors connected to the IMPLANTABLE DEVICE 21108. The data (2194) presented may include but not limited to the Body Temperature, Blood Ph value, Blood pressure, Blood Glucose level, etc.

### O. Wireless Power Transfer

US 2014/0055088 describes a method for wireless charging of implant devices. A backscatter from the communication coil is used to indicate the best frequency for efficient charging. Thus, the external transmitter can adjust the frequency for wireless power transmission based on a basic feedback mechanism using backscattered information.

US 2014/0084855 describes wireless power transmission and data transmission to the implant (from the outside to the implant). The backscatter signal is received by an external system and processed to control the implant impedance matching unit or to change the frequency of the external device. In both cases, this is done to obtain maximum wireless power transfer.

### 1. Wireless Power Transfer Using Inductive Coupling.

In some embodiments, a wireless power transfer (WPT) for battery charging using inductive links can be utilized to charge the battery of the implanted device. When the battery has expended (or nearly expended) its capacity, the battery can be recharged via inductive coupling from an external power source temporarily positioned on the surface of the skin. Wireless power transfer (WPT) technology can increase the degree of freedom of devices that have been supplied with power through the wire and can solve both the inconvenience and the safety problem of the wire at the same time.

The energy transfer through the use of inductive coupling involves the placement of two coils positioned in close proximity to each other on opposite sides of the cutaneous boundary. The internal coil, or secondary coil, is part of or otherwise electrically associated with the implanted medical device. The external coil, or primary coil, is associated with the external power source or external charger, or recharger. The primary coil is driven with an alternating current. A current is induced in the secondary coil through inductive coupling. This current can then be used to power the implanted medical device or to charge, or recharge, an internal power source, or a combination of the two.

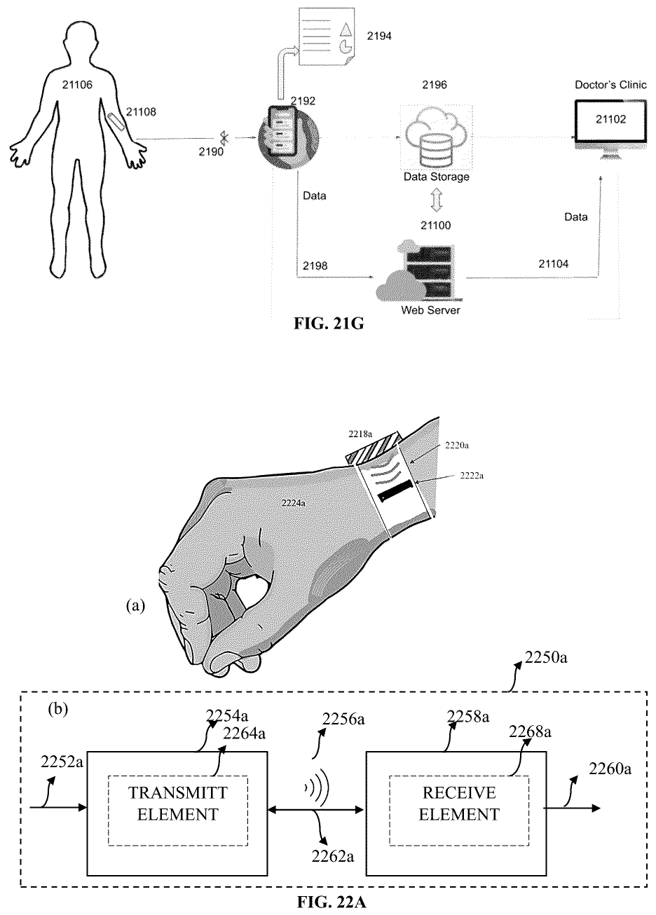

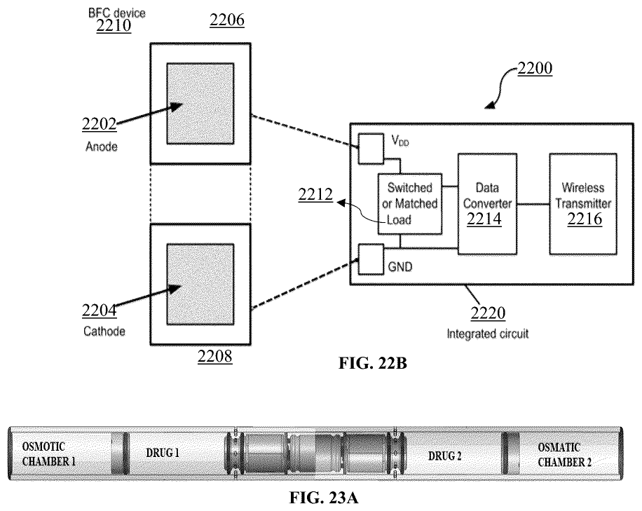

Referring to FIG. 22A, (a) depicts an arrangement for power supply to the implantable device through inductive coupling of coils as described above. 2218a is the watch type wearable device with the primary coil or the power source, 2222a is an implantable device which is equipped with the secondary coil, implanted into a body organ 2224a. 2220a depicts the inductive field.

Referring to FIG. 22A, (b) depicts a functional block diagram of an example of a wireless power transfer system 2250a. Input power 2252a may be provided to a transmitter 2254a from a power source (not shown in this figure) to generate a wireless (e.g., magnetic or electromagnetic) field 2256a for performing energy transfer. A receiver 2258a may couple to the wireless field 2256a and generate output power 2260a for storing or consumption by a device (not shown in this figure) that is coupled to receive the output power 2260a. The transmitter 2254a and the receiver 2258a are separated by a non-zero distance 2262a. The transmitter 2254a includes a power transmitting element 2264a configured to transmit/couple energy to the receiver 2258a. The receiver 2258a includes a power receiving element 2268a configured to receive or capture/couple energy transmitted from the transmitter 2254a.

The transmitter 2254a and the receiver 2258a may be configured according to a mutual resonant relationship. When the resonant frequency of the receiver 2258a and the resonant frequency of the transmitter 2254a are substantially the same, transmission losses between the transmitter 2254a and the receiver 2258a are reduced compared to the resonant frequencies not being substantially the same. As such, wireless power transfer may be provided over larger distances when the resonant frequencies are substantially the same. Resonant inductive coupling techniques allow for improved efficiency and power transfer over various distances and with a variety of inductive power transmitting and receiving element configurations.

Wireless power transfer efficiency from outside a patient to an implanted device inside a patient may be improved, e.g., by using different power transfer techniques for transcutaneous power transfer and patient-internal power transfer. Less power may be transferred transcutaneously to power an implanted device wirelessly than with prior techniques. More diffuse power transfer may be used transcutaneously to power an implanted device wirelessly than with prior techniques. What battery size is used in an implanted device may be dependent upon a depth of the implanted device in an entity (e.g., a patient), and/or distance from a power source for powering the implant. A wired connection between implants may be avoided, and thus risk of breakage of the wired connection, risk of infection, and risk of a wired connection acting as an antenna and heating surrounding tissue (e.g., during an MRI of an entity containing the implants), may be avoided. Multiple implants, including a deep implant (an implant disposed, for example, 5 cm or more from a surface of an entity containing the deep implant), may be inserted into a patient using minimally-invasive surgery. Flexibility for a shallow implant placement may be improved. Advantages of ultrasound power transfer (e.g., lower attenuation, higher permitted intensity than with radio-frequency energy) may be realized without using a gel or requiring physical contact of a power source with a patient. Energy may be better directed toward an implant, e.g., a deep implant. Required alignment of a transmitter and a receiver in a wireless power system may be reduced. Other capabilities may be provided and not every implementation according to the disclosure must provide any, let alone all, of the capabilities discussed. Further, it may be possible for an effect, noted above, to be achieved by means other than that noted, and a noted item/technique may not necessarily yield the noted effect.

### Wireless Power Transfer Using Magnetic Induction

In some embodiments, the implantable device may be recharged by magnetic induction. In an example, the implanted device may be operated on a single rechargeable cell, or a dual power source system, and the rechargeable complement may be recharged by magnetic induction as described in U.S. Pat. No. 5,411,537 included as incorporation by reference in its entirety.

In some embodiments, a current with a sinusoidal waveform is applied to a resonant circuit comprising a primary coil and a capacitor. Current is induced in a secondary coil attached to the implanted medical device. Two solid-state switches are used to generate the sinusoidal waveform by alternately switching on and off input voltage to the resonant circuit. The batteries are charged using a charging protocol that reduces charging current as the charge level in the battery decreases. An alignment indicator is also provided to ensure proper alignment between the energy transmission device and the implanted medical device. This power transfer technique is described in U.S. Pat. No. 5,690,693 and included in this specification as incorporation by reference in its entirety. Similar technologies are described in U.S. Pat. Nos. 6,324,430, 6,505,077, 5,411,537, 5,690,693, 6,324,430 and 6,505,077 and included in this application as incorporation by reference in their entirety.

### P. Device Configured for Self-Powering

In an embodiment, the implantable device is self-powered. The implantable device comprises a subsystem for sensing of metabolites while using the power from a biofuel (e.g., the metabolite) to directly power an analog-to-digital converter and wireless transmitter, without requiring any external power source is disclosed. The subsystem comprises an electronic circuit, including a signal converter and a switched or matched impedance load; an anode, including a first nanocomposite and an enzymatic layer and a cathode, including a second nanocomposite and electrically coupled to a ground voltage terminal of the electronic circuit. The anode is electrically coupled to a power supply voltage terminal of the signal converter, when connected through the switched or matched impedance load of the electronic circuit and is configured to interact with an analyte in a fluid, the analyte including glucose or lactate. The electrical energy is generatable from transformation of the analyte to a derivative substance based on electrochemical reactions across the anode and cathode. The electronic circuit is configured to control and utilize generated power from the electrochemical reactions across the anode and cathode to supply the generated power to components of the electronic circuit. The electronic circuit is operable to translate the electrical energy as transmittable digital data associated with a concentration of the analyte.

Referring to FIG. 22B, the biofuel cell powered electronic circuit 2200 which is self-powered by a biofuel (e.g., glucose or lactate) and modulated at a low voltage to power an integrated circuit connected to the biofuel cell contingent. The BFC-powered electronic circuit 2200 includes one or more integrated circuits 2220, also referred to as an electronic circuit 2220, coupled to a biofuel cell device 2210. The biofuel cell device 2210 includes an anode 2202 including a conductive electrode that is coupled to a first substrate 2206, where an enzymatic layer is formed on the conductive electrode of the anode 2202 and configured to electrochemically interact with the biofuel (e.g., glucose or lactate) in a fluid. The anode 2202 is electrically coupled to a power supply voltage terminal (e.g., V_{DD}) of the electronic circuit 2220. The biofuel cell device 2210 includes a cathode 2204 including a conductive electrode, e.g., which can include a nanocomposite, that is coupled to a second substrate 2208. The cathode 2204 is electrically coupled to a ground voltage terminal (e.g., GND) of the electronic circuit 2220. In some embodiments, the first substrate 2206 and the second substrate 2208 is a single substrate, where the anode 2202 and the cathode 2204 are spaced apart on the single substrate. In an example, the V_{DD} of the electronic circuit 2220 is set to a voltage of 0.25 V to 0.6 V (e.g., 0.3 V to 0.4 V in some implementations), which is near the open circuit voltage of the BFC device 2210. The electronic circuit 2220 includes a data converter 2214 to translate the power generated by the biofuel cell device 2210 to a transmittable data. For example, in some embodiments, the data converter 2214 includes a delta-sigma modulation analog-to-digital (DSM ADC) converter or a ring oscillator, which is able to operate directly from the power generated by the enzymatic layer to use a maximum power point of the enzymatic BFC device 2210 to infer the biofuel concentration in the fluid. In this manner, for example, the electronic circuit 2220 is operable to translate the electrical energy as transmittable digital data that is indicative of a concentration of the analyte (e.g., biofuel) in the fluid. To control the operation of the data converter 2214, for example, the electronic circuit 2220 includes a switched or matched load 2212 coupled between the anode 2202 and the data converter 2214 to control supply of the electrical energy (e.g., electrical current) extracted from the biofuel to the electronic circuit 2220. For example, the switched or matched load 2212 can connect the anode 2202 of the biofuel cell device 2210 to the data converter 2214, in some implementations, to establish a supply voltage at a maximum power point in a range, e.g., of 0.25 V to 0.6 V (in some examples, to the maximum power point that can include a range of 0.3 V to 0.4 V). Also, for example, in some implementations where embodiments of the data converter 2214 include the ring oscillator, the switched or matched load 2212 can connect the anode 2202 to the ring oscillator to establish a supply voltage that is set by a fixed load.

In implementations of the BFC-powered electronic circuit 2200, for example, the electronic circuit 2220 is configured to use power generated while the biofuel (e.g., glucose or lactate) is being decomposed by the enzymatic layer of the anode 2202 while also determining information about the biofuel (e.g., concentration of the biofuel in the fluid), thereby functioning as a self-powered biosensing system and bioelectronic system that can be employed in a variety of bio-related applications. In some embodiments, the electronic circuit 2220 can include a wireless transmitter 2216 in electrical communication to the data converter 2214, which can transmit the converted digital signals as data.

In some example embodiments, the electronic circuit 2220 can include analog signal conditioning circuitry, an analog-to-digital converter, or a wireless transmitter, or a combination of any two or more of the analog signal conditioning circuitries, the analog-to-digital converter, and the wireless transmitter.

### Q. Embodiments for Addressing Foreign Body Response (FBR)

One of the problems faced by the patient after implantation of the device in a patient is that the immune system of the patient attacks the implantable, forming a thick layer of scar tissue that potentially can block the drug release.

The following embodiments are examples to address the issue of foreign body response:

In an embodiment, the implantable is incorporated into a soft robotic device. The soft robotic device is repeatedly inflated and deflated for five minutes every 12 hours or at a redetermined intervals, to create mechanical deflection which can prevent immune cells from accumulating around the device. The mechanical actuation is designed to drive away immune cells called neutrophils. These cells initiate the process leading to scar tissue formation.

In an embodiment, the device may be a two-chambered device made of polyurethane, a plastic that has similar elasticity to the extracellular matrix that surrounds tissues. One of the chambers is configured to act as a drug reservoir, and the other chamber is configured to act as a soft, inflatable actuator. Using a controller, either internal or external, the actuator is stimulated to inflate and deflate on a specific schedule. In an embodiment, the actuation every 12 hours, for five minutes at a time. This mechanical actuation is configured to drive away immune cells called neutrophils, the cells that initiate the process that leads to scar tissue formation.

In an embodiment, the device uses biocompatible coatings and hydrogels for reducing foreign body response and fibrosis. Zwitterionic polymers or biocompatible polymers with improved properties for cell encapsulation, coating of devices, or a combination thereof as described in US patent U.S. Ser. No. 10/730,983B2, which is incorporated in its entirety.

In an embodiment, the implantable device is coated with microporous surface layers with macrotopographic features that improve bio-integration at the interface of the implantable devices and the surrounding tissue as described in US patent U.S. Pat. No. 8,647,393B2, which is incorporated in its entirety. In an embodiment, it is an implantable device comprising: a device body; and a textured surface layer overlying the device body, wherein the textured surface layer comprises one or more granules of a microporous biomaterial, the granules forming a surface macrotopography that includes a plurality of peaks and valleys, each peak having a height of between about 100 micrometers and about 2000 micrometers, and wherein each granule comprises a plurality of interconnecting pores having a mean pore diameter of between about 5 and 100 micrometers, and any two adjacent pores are connected by a throat, a mean throat diameter of the throats being between 5 and 50 micrometers, and at least two adjacent peaks define a valley, the valley having a floor from which the heights of the adjacent peaks are measured.

In an embodiment, the system further comprises a site loss mitigating agent comprising rapamycin that inhibits at least one of: coagulation at the single site of infusion, inflammation at the single site of infusion, and encapsulation of the cannula at the single site of infusion. Methods and devices on mitigating site-loss and/or occlusion are included in their entirety from US patent application US20200138852A1, which is incorporated in its entirety.

In an embodiment, the implantable device comprises a biointerface membrane, wherein the membrane is configured to modify an in vivo tissue response by a porous architecture and by incorporation of a bioactive agent in the membrane as described in US patent application US20200375515A1, which is incorporated in its entirety.

In one aspect, the present invention relates to a device comprising an implantable device which has an in vivo functionality, as well as a protective material in close proximity to the surface of the implantable device. The protective material prevents or reduces degradation or interference of the implantable device due to inflammation reactions and/or foreign body response. Further, the protective material can comprise a metal or metal oxide which catalytically decomposes or inactivates in vivo reactive oxygen species or biological oxidizers. In another aspect, the present invention relates to a device comprising an implantable device which has an in vivo functionality as well as a protective coating deposited on the surface of the implantable device. The protective coating prevents or reduces degradation or interference of the implantable device due to inflammation reactions and/or foreign body response. Further, the protective coating can comprise a metal or metal oxide which catalytically decomposes or inactivates in vivo reactive oxygen species or biological oxidizers. Aspects of protective coatings on implantable devices are herein incorporated from the U.S. Pat. No. 9,681,824B2, which is incorporated in its entirety.

In an embodiment, the implantable device comprises, in part, an encapsulation sensor. The encapsulation sensor comprises at least two electrodes and a circuit configured to sense impedance between the electrodes. Cell accumulation and fibrous capsule growth causes an increase in impedance which is used to detect the foreign body response. Functionality of the sensor can be evaluated based at least in part on the sensed impedance. The encapsulation sensor and the method of determining foreign body response according to an embodiment are as described in US application, US20080081965A1, which is incorporated in its entirety.

### R. Drug Delivery Implantable Device Comprising Sensor/s

In some embodiments, a system comprising a drug delivery implantable device and a sensor is described herein. The sensor may be a conductivity/resistivity sensor for measuring ionic concentration of a compound; an optical sensor such as a CCD or CMOS image sensor; an electrical, electrochemical, or chemical sensor for measuring characteristics such as impedance, temperature, pH, enzymatic activity, etc.; a mechanical sensor; a biochemical sensor for measuring the presence and/or levels of analytes; an acoustic sensor such as an ultrasound; a light sensor such as a photodiode; or other sensor within the purview of those skilled in the art for measuring and/or identifying a physiological condition or state, such as tissue perfusion, tissue ischemia and/or reperfusion, pH, bacterial load, temperature, pressure, protein or bioactivity factors, metabolic analytes, and other biomarkers or parameters of interest. The system can be multi-functional and one of a mono-sensing system, bi-sensing system or a multi-sensing. The system can detect one or more biomarkers of one or more disease conditions. The one or more disease conditions comprise at least one of diabetes, cardiac malfunction, liver malfunction, a brain related disorder, a kidney malfunction, cancer, oxygen saturation, drug addiction, pancreas malfunction. In some embodiments, the system can be used for monitoring of surgical and medical treatments of disease and body ailments of a patient, such as necrosis, infection, and cancer. In an example, the system of the present disclosure may be utilized in the detection of infection, metabolic disorder, or other abnormal or non-ideal conditions of wound healing.

### Sensor

In an embodiment, sensors for continuous analyte measurement are provided that could be implanted within the body. For an implantable sensor, small size reducing the likelihood of thrombus formation upon implantation and impingement of the sensor structure on adjacent blood vessels, and thus, maximizing fluid flow to the sensor. One manner of reducing the size or surface area of at least the implantable portion of a sensor is to provide a sensor in which the sensor's electrodes and other sensing components and/or layers are distributed over both sides of the sensor, thereby necessitating a narrow sensor profile. Examples of such double-sided sensors are disclosed in U.S. Pat. No. 6,175,752, U.S. Patent Application Publication No. 2007/0203407, now U.S. Pat. No. 7,826,879, and U.S. Provisional Application No. 61/165,499 filed Mar. 31, 2009, the disclosures of each of which are incorporated herein by reference for all purposes.

In one embodiment, the implantable device features an exterior with a printed pH sensor. This sensor utilizes biocompatible materials and printing technologies on flexible films. Various technologies, including bioresorbable nanostructured sensors, implantable pH microneedle sensors, and electrochemical pH sensors, enable accurate and continuous pH measurements.

### Analyte Measuring Sensor

It would also be highly advantageous to provide continuous analyte monitoring sensors that are substantially impervious to, or at least minimize, spurious low readings due to the in vivo environmental effects of subcutaneous implantation, such as ESA and night-time dropouts. Of particular interest are analyte monitoring devices and systems that are capable of substantially immediate and accurate analyte reporting to the user so that spurious low readings, or frequent calibrations, are minimized or are nonexistent.

Analytes that may be monitored include, but are not limited to, acetyl choline, amylase, bilirubin, cholesterol, chorionic gonadotropin, creatine kinase (e.g., CK-MB), creatine, creatinine, DNA, fructosamine, glucose, glutamine, growth hormones, hormones, ketone bodies, lactate, oxygen, peroxide, prostate-specific antigen, prothrombin, RNA, thyroid stimulating hormone, and troponin. The concentration of drugs, such as, for example, antibiotics (e.g., gentamicin, vancomycin, and the like), digitoxin, digoxin, drugs of abuse, theophylline, and warfarin, may also be monitored. In those embodiments that monitor more than one analyte, the analytes may be monitored at the same or different times.

It is envisioned that the sensor utilized in the embodiments described herein can functionally persist in vivo for a period of years. Although a direct estimate of the duration of the functionality of a specific sensor module depends on the specific embodiment, some embodiments envisioned herein are estimated to provide information regarding analytes for a period of no less than 5 years after the implantation surgery, while some are envisioned to provide information regarding analytes for approximately 5 to approximately 10 years after implantation and initiation of use.

In an embodiment, analyte measuring sensor may have a data processing unit connectable to the sensor, and a primary receiver unit which is configured to communicate with the data processing unit via a communication link. In certain embodiments, the primary receiver unit may be further configured to transmit data to a data processing terminal to evaluate or otherwise process or format data received by the primary receiver unit. The data processing terminal may be configured to receive data directly from the data processing unit via a communication link which may optionally be configured for bi-directional communication. Further, the data processing unit may include a transmitter or a transceiver to transmit and/or receive data to and/or from the primary receiver unit and/or the data processing terminal and/or optionally the secondary receiver unit. U.S. Ser. No. 10/827,954B2 is incorporated herein by reference in its entirety.

In an embodiment, the system may embody the sensors to monitor the proper functioning of various organs not limited to pancreas, heart, kidney, brain, etc. In an embodiment, these sensors could have access to the patients' data through remote monitoring.

It would also be desirable to provide sensors for use in a continuous analyte monitoring system that have negligible variations in sensitivity, including no variations or at least not statistically significant and/or clinically significant variations, from sensor to sensor. Such sensors would have to lend themselves to being highly reproducible and would necessarily involve the use of extremely accurate fabrication processes.

In some embodiments, the implantable sensors function for an operational lifetime, and, at some point, after the operational lifetime, are to be resorbed by the body in such a manner so as to avoid having to remove any part of the sensor. The implantable sensor may comprise a substrate, an electronic device, and a barrier layer wherein the substrate, the electronic device, and the barrier layer have a bioresorption rate during use to provide controlled bioresorption of the implantable and bioresorbable sensor, configured so that the implantable and bioresorbable sensor operates for an operational lifetime and has a bioresorption lifetime that is greater than the operational lifetime, and no detectable portion of the implantable and bioresorbable sensor remains at an implantation site after the bioresorption lifetime.

### S. Sensors for the Device and/or System

In another embodiment, the device/system comprises one or more primary sensors wherein the one or more primary sensors comprise a concentration sensor to measure a drug concentration in the body of the subject.

In another embodiment, the device/system comprises one or more secondary sensors wherein the one or more primary sensors comprise a concentration sensor to measure a drug concentration in the body of the subject.

In another embodiment, the device/system comprises one or more primary sensors wherein the one or more primary sensors comprise a diseases marker sensor.

In another embodiment, the device/system comprises one or more secondary sensors wherein the one or more primary sensors comprise a diseases marker sensor.

The table below provides one or more sensors that can be used with the implantable device/system:

| | | Relevant | Drug | Relevant | |
|---|---|---|---|---|---|
| | Markers of | sensor for | corresponding | sensor for | |
| Diseases | the Diseases | biomarker | to the disease | the drug | Resource |
| Diabetes | Glucose and | Continuous | Metformin; | Electrochemical | US20080161666A1 |
| | Gluconic acid | glucose | Sulfonylureas; | biosensor; | U.S. Pat. No. 11,690,577B2 |
| | concentration | monitoring | Thiazolidinediones; | (CN114994153A) | U.S. Pat. No. 7,766,830B2 |
| | Blood | sensor; | Dipeptidyl | Diffraction | KR100602952B1 |
| | Glucose | Flash | Peptidase-4 | based sensor | KR102315843B1 |
| | Levels | glucose | Sodium-glucose | CN114609123A | US2012197148A1 |
| | HbA1c | monitoring | co-transporter2; | Analyte sensor | KR20230014498A |
| | Ketones | sensor; | GLP-1 Receptor | WO2007136390A1 | U.S. Pat. No. 8,615,282B2 |
| | Insulin | | Agonists; | | US20120059232A1 |
| | Levels | | Insulin | | doi: 10.1109/ |
| | Heart Rate | | | | TBCAS.2009.201684. |
| | Variability | | | | doi: 10.1109/ |
| | (HRV) | | | | TBCAS.2009.201684. |
| | Skin | | | | doi.org/10.3390/ |
| | Temperature | | | | photonics6020071 |
| | | | | | doi: 10.1016/ |
| | | | | | j.bios.2016.03.024. |
| | | | | | doi: 10.1109/ |
| | | | | | TBCAS.2009.201684. |
| Renal | Glomerular | Wearable | Angiotensin- | Optical sensor; | CA2425337C |
| diseases | filtration rate; | devices, | Converting | | |
| | Urine Protein; | such as | Enzyme (ACE) | | |
| | Creatinine; | smartwatches, | Inhibitors; | | |
| | Blood Urea | wristbands, | Angiotensin II | | |
| | Nitrogen; | or patches, | Receptor Blockers | | |
| | Electrolyte | capable of | (ARBs); | | |
| | Imbalances; | analyzing | Diuretics; | | |
| | Renal Blood | sweat or | Immunosuppressive | | |
| | Flow | interstitial | Drugs; | | |
| | | fluid | Antihypertensive | | |
| | | | drugs; | | |
| | | | Erythropoiesis- | | |
| | | | Stimulating | | |
| | | | Agents (ESAs); | | |
| | | | Phosphate binders | | |
| Hypertension | Uric acid | — | — | Dual-mode | https://doi.org/10.1016/ |
| | | | | colorimetric | j.microc.2020.105865 |
| | | | | and | |
| | | | | fluorometric | |
| | | | | nanosensor | |
| Cardiovascular | Blood | — | warfarin | Electrochemical | https://doi.org/10.3390/ |
| diseases (blood | prothrombin | | | sensors based | chemosensors10020044 |
| vessel disease, | time; | | | on ion selective | |
| atrial fibrillation, | International | | | electrode | |
| stroke, pulmonary | Normalized | | | technology | |
| embolism, pulmonary | Ratio (INR) | | | optical sensors, | US20170281771A1 |
| hypertension, | | | | ultrasonic sensors, | |
| and deep vein | | | | pressure sensors | |
| thrombosisenous and | | | | | |
| arterial thromboembolic | | | | | |
| disorder) | | | | | |
| Cancer | proteins, mRNA, | | | Multiplexed | U.S. Pat. No. 10,603,650B2 |
| | cytokines, | | | surface | doi: 10.1039/c5an01861g |
| | | | | enhanced | doi: 10.3390/s21041125. |
| | | | | Raman sensors | |
| HIV | HIV RNA | — | Long acting | Co-encapsulated | doi: 10.1016/ |
| | viral load | | antiretrovirals | antiretrovirals | j.ebiom.2022.104330. |
| | | | | (ARVs) with | |
| | | | | ingestible | |
| | | | | sensor (IS) | |
| Depression | elevated C-reactive | | neurotransmitter, | Aptamer-based | US20190231240A1 |
| | protein in blood | | hormone, | sensor, disposable | doi.org/10.3390/ |
| | | | pharmaceutical | voltammetric | ijms24108480 |
| | | | drug, or toxin, | MIP based sensor | |
| | | | Trazodone (TZD), | | |
| | | | long acting | | |
| | | | anti-psychotics | | |
| Ocular | Intraocular | | | Implantable | EP3659495B1 |
| disease | pressure, | | | intraocular | US20120316461A1 |
| | IL-12p70; | | | physiological | https://doi.org/10.1021/ |
| | Acetyl carnitine | | | measurement | acssensors.1c00370 |
| | (C2); IFN-γ; | | | device, contact | DOI: 10.1039/D1AN01244D |
| | GM-CSF; IL-5; | | | lens sensor | |
| | carbonic anhydrase, | | | | |
| | lipase and | | | | |
| | antioxidants; BDNF, | | | | |
| | TFN-α; alpha-1 | | | | |
| | antichymotrypsin, | | | | |
| | Cystatin SA; | | | | |
| | protein S100-A4; | | | | |
| | Keratin (typeII) | | | | |
| | etc., Lysozyme C; | | | | |
| | lacritin, | | | | |
| | lipophilin A, | | | | |
| | Ig lambda chain; | | | | |
| Acute abdominal | Abnormal intra- | | | Tubular pressure | US20120316461A1 |
| syndrome | abdominal pressure | | | sensing device | |
| | (IAP) | | | | |
| Neurodegenerative | Acetylcholine (Ach), | | Dopamine, | Carbon | https://doi.org/10.1016/ |
| Diseases | dopamine (DA) and | | acetylcholine, | Nanotube-Based | j.biochi.2017.12.015 |
| | norepinephrine (NE) | | levodopa carbidopa | Fluorescent | https://doi.org/10.3390/ |
| | | | | Nanosensors, | bios13050499 |
| | | | | Optode-Based | doi: 10.1146/annurev- |
| | | | | Nanosensors | anchem-061417-125747 |
| Male | Hormone Levels; | Wearable | Phosphodiesterase | | |
| reproductive | Prostate-Specific | biosensors; | type 5 (PDE5) | | |
| organs related | Antigen | Saliva-Based | inhibitors; | | |
| aliments | | sensors; Breath | Alprostadil; | | |
| | | analysis | Alpha-1 blockers; | | |
| | | sensors; | 5-alpha-reductase | | |
| | | | inhibitors; | | |
| | | | leuprolide (Lupron) | | |
| | | | and bicalutamide | | |
| | | | (Casodex); | | |
| | | | docetaxel (Taxotere) | | |
| | | | or cabazitaxel | | |
| | | | (Jevtana); | | |
| | | | Clomiphene citrate; | | |
| Autoimmune | Antigliadin Abs | | | immunosensors | doi: |
| diseases [Celiac | (AGA) and anti- | | | | 10.3390/bios9010038. |
| disease (CD)] | transglutaminase | | | | |
| | Abs (anti-tTG) | | | | |
| Asthma | FeNO (inhaled | | Corticosteroids and | Sensor based | doi: |
| | corticosteroid | | short-acting | inhaler | 10.1542/peds.2020-1330. |
| | (ICS) adherence) | | β-agonist | | |
| | | | medications | | |
| Infectious | tracheal cytotoxin | | | multiplexed | U.S. Pat. No. 10,603,650B2 |
| disease | (TCT), Hemozoin | | | surface enhanced | U.S. Pat. No. 8,774,884B2 |
| | nanoparticles | | | Raman sensors, | https://doi.org/10.3390/ |
| | | | | Electromagnetic | s20143953 |
| | | | | energy sensor, | https://doi.org/10.3390/ |
| | | | | Aptamer based | nano11040840 |
| | | | | sensor | |

### 1.1 Wearable Device to Monitor Oxygen.

In an embodiment, the system could further have an implantable optical probe for continuous monitoring of oxygen saturation in a body. This process can help in early diagnosis of and intervention for anastomotic thrombosis. In an embodiment, the device having an implantable optical probe for continuous monitoring of oxygen saturation in a body including but not limited to Doppler systems, cutaneous O₂-sensing probes, and fluorine magnetic resonance imaging methods. In an embodiment, implantable optical probe is wireless, miniaturized, minimally invasive near-infrared spectroscopic system designed for uninterrupted monitoring of local-tissue oxygenation.

### 1.2 Wearable Device to Monitor Blood Pressure.

In an embodiment, the system includes an implantable continuous blood pressure monitoring system. The implantable continuous blood pressure could be based on piezoelectric transducer with a low power control chip able to transmit pressure measurements continuously for 5-10 s. In an embodiment, the system includes CardioMEMS™ heart failure sensor based on a (MEMS) pressure-sensitive capacitor. In an embodiment, the implantable sensor may be electromagnetically coupled to an external antenna, which could power the devices and subsequently capture its resonant frequency, which is related to the arterial pressure. In an embodiment, an implantable sensor could be coupled with a subcutaneous antenna coil that could be implanted by means of transseptal puncture and interacted by means of radio frequency (RF) excitation transmitted by an external module. In an embodiment, the implantable blood pressure monitor is an intra-arterial pressure measurement. In another embodiment, the implantable blood pressure monitor is an extra-arterial blood pressure monitor.

### 1.3 Wearable Device to Monitor Blood Activity.

Anaemia is very commonly seen in patients. Human RBC has a typical life span of 120 days, and in general the body replaces about 1% of these cells through the process of erythropoiesis daily. The kidneys play a main role in this process via erythropoietin (Epo). This process could be affected during kidney disease. In an embodiment, the implantable device could be configured to monitor function of kidney.

In an embodiment, the system embodies sensors for continuous monitor hemodynamic activities. An implantable device could provide a long-term serial hemodynamic measurement which may provide an insight regarding pathophysiological mechanisms and chronic responses to treatment paradigms. In an embodiment, the sensor may be a dynamic pressor sensor having an integrated circuit, sensor port, optical barrier, red infrared diodes, sensor diaphragm and piezoelectric crystal, or an absolute pressure sensor having transducer capacitor, reference capacitor and integrated circuit and sensor diaphragm, as described in Pacing and clinical electrophysiology, 28(6), 573-584.

In an embodiment, the system may further have a peripheral memory patch apparatus for attachment to a patient's skin including a high-capacity memory for storing physiologic data uplinked from an implantable medical device. A resilient substrate provides support for a memory, microprocessor, receiver, and other electronic components. The substrate flexes in a complimentary manner in response to a patient's body movements. The substrate is affixed to the patient's skin with the use of an adhesive which provides comfort and wearability. The low-profile peripheral patch apparatus is preferably similar in size and shape to a standard bandage, and may be attached to the patient's skin in an inconspicuous location. A status indicator provides for a visual, verbal, or tactile indication of the operational status of the peripheral memory patch. Uplinking of physiologic telemetry data from the internal memory of an implantable medical device to the peripheral memory patch is initiated in response to a transfer signal produced by the peripheral memory patch. The transfer signal may be generated by the implantable medical device or upon actuation of a switch by the patient. Various telemetry techniques including radio frequency, acoustic, and body bus telemetry techniques, may be employed to transfer information between the implantable medical device and the peripheral memory patch. In an embodiment, this provides expanded memory resources for the purpose of storing physiologic and other data acquired or produced by an implantable medical device, providing for the continuous storage of physiologic data acquired by an implantable medical device over an extended period of time, such as on the order of days, increasing the ease by which large amounts of physiologic data may be acquired, providing a comfortable and inconspicuous apparatus for effectively extending the memory capacity of an implantable medical device, eliminating the need for a patient to make repeated visits to a physician's office for the sole purpose of extracting physiologic data stored in an implantable medical device, downloading patch programs which are programmed into IMD random access memory (RAM) to allow functional changes to the implanted device for problem patients, to test and evaluate algorithms, to gather data for research activities, and the like, and allowing all or nearly all of the RAM memory to be used for downloadable patches, with the diagnostic data being stored in the peripheral memory patch. U.S. Pat. No. 6,200,265B1 is incorporated by reference in its entirety.

### 1.4 Wearable Device to Monitor Heart Rate.

In an embodiment, the implantable device can be self-powered by harvesting mechanical energy from the heart. It could continuously monitor multiple cardiac signals such as heart rates, respiratory rates, ECGs, and the estimated BP. In an embodiment, invasive epicardial sensors are included in the implantable device that are also capable of cardiac mapping with a high spatiotemporal resolution. In an embodiment, the device may have a chip capable of continuously monitoring the concentration of a variety of molecules, including glucose and cholesterol, as well as pH levels and body temperature.

### 1.5 Wearable Device to Monitor Nervous System.

In an embodiment, a vagus nerve stimulator delivered with an implanted device to treat rheumatoid arthritis. The vagus nerve runs from the brainstem down to the abdomen through the neck and chest and is a main channel for the communication of signals between the brain and the body. The device is implanted via keyhole surgery into the stomach, targeting several spots on the vagus nerve in the abdomen. In an embodiment, electrical pulses from the implantable device could treat diseases which are being treated with potent and relatively expensive drugs currently.

### T. Smart Implantable Device

In some embodiments, the implantable device comprises an analytical component coupled to the sensor component and a drug delivery component coupled to the analytical component for delivering a drug to a host. The sensor can measure and send signals such as a sensor temperature signal, an impedance signal, an oxygen signal, a pressure signal, or a background signal to the microprocessor and/or microcontroller of the implantable device. The device can monitor one or more parameters comprising internal body temperature, hemodynamic, internal body analytes, and disease biomarkers. In some embodiments, the system comprises a first primary sensor that can detect clinical context information and a secondary primary sensor that can detect a device failure.

### 1. Drug Dosing Based on Real Time Measurement of Glucose Level

In some embodiments, the system comprises an implantable device further comprising a blood glucose monitoring sensor. The blood glucose monitoring sensor can be an enzymatic-based sensor capable of selectively and sensitively monitoring glucose in the subcutaneous tissues under low oxygen tensions as described in US20220079474A1 which is incorporated herein by reference in its entirety. The enzymatic-based sensor includes an oxygen transport region comprising a first reversible oxygen binding protein, an oxygen permeable first surface in communication with an external environment, and an oxygen permeable second surface which is impermeable to the target analyte; a target analyte reaction region in communication with the oxygen transport region at the oxygen permeable second surface, wherein the target analyte reaction region comprises a target analyte oxidase enzyme, and a target analyte-permeable surface; and a sensing region comprising at least one detector probe in communication with the target analyte reaction region. Typically, a target analyte and oxygen impermeable surface is located in the enzymatic-based sensor such that the sensing region is in between the target analyte and oxygen impermeable surface and the oxygen transport region. The target analyte can be, for example, glucose, galactose, lactose, peroxide, cholesterol, amino acids, alcohol, or lactic acid. In certain illustrative examples, the target analyte is glucose and the sensor is a glucose sensor. For example, the glucose sensor can be an implantable glucose sensor such as a transcutaneous glucose sensor.

Accordingly, provided herein is the first primary sensor as a glucose sensor that includes an oxygen transport region, that includes a reversible oxygen binding protein, an oxygen permeable first surface and an optional oxygen permeable surface in communication with an external environment, and an oxygen permeable second surface, sometimes referred to herein as the oxygen injector; a glucose reaction region in mass transport communication with the oxygen transport region at the oxygen permeable second surface, wherein the glucose reaction region includes, the reversible oxygen binding protein, a glucose oxidase enzyme and a glucose permeable surface, sometimes referred to herein as the glucose inlet, and a sensing region in optical or electrical communication with the glucose reaction region, wherein the sensing region includes a probe, such as an oxygen probe, a glucose probe, or a probe that binds to, or is otherwise affected by, a product of the reaction of oxygen and glucose. The sensing region can be a region within the reaction region, or a region sufficiently in contact with the glucose reaction region to represent the spatial and temporal profile of glucose or other target analyte, oxygen, and/or reaction product within the reaction region, that is interrogated by one or more detector probes, which are individual sensing elements within a sensing interface. Furthermore, the device can have an axis of symmetry. The oxygen transport region is an exemplary stabilized oxygen transport matrix of the present invention. Communication between the oxygen transport region and the glucose reaction region typically occurs across a surface where the oxygen transport region and glucose reaction region are in contact. The communication between the oxygen transport region and the glucose reaction region can be any type of communication that involves diffusion and permits the transport of oxygen from the oxygen transport region into the glucose reaction region. The communication can be, for example, the movement of liquid, gas, and/or ions from the oxygen transport region to the glucose reaction region. A sensor for in vivo continuous multiple analyte monitoring is disclosed in US20220079474A1, included in its entirety in this application.

In some embodiments, the system comprises an implantable device further comprising a passive transmitter tag device. The passive transmitter tag device comprises (a) a non-linear impedance element or time-varying element for introducing harmonics or frequency-shifted signal components into a back-emf produced by a sinusoidal carrier signal; (b) an energy harnessing element; (c) sensor/transducer for monitoring a patient condition, wherein said sensor is operably connected to said non-linear impedance element or time-varying element. The sensor/transducer may be a pressure monitor, a pH monitor, a temperature monitor, or a blood glucose monitor. The energy harvesting element may comprise a rectifier and capacitor. The non-linear impedance element may be a bipolar transistor, a field-effect transistor or a voltage-controlled oscillator. The time-varying element may be a switch or a mixer. The mixer may be operably connected to receive (i) an input from a voltage-controlled oscillator, and (ii) an input driven by induced voltage from the carrier signal.

In another embodiment, there is provided a reader device comprising (a) a signal source; (b) a wave signal generator; (c) a wave signal detector; and (d) a signal processing unit. The device may further comprise a filtering circuit, such as a high pass filter, a notch filter or a resonant LC tank. The device may further comprise an analog to digital converter. The signal processing unit may employ a finite impulse response-matched filter, or a frequency domain method such as a short-time Fourier Transform, a windowed fast Fourier Transform, or a spectrogram. The signal processing unit may employ a look-up table. Also provided are systems for transmitting information from a sensor comprising the transmitter tag device and the reader device described above. Still another embodiment is a method for transmitting information from the transmitter tag to the reader in the aforementioned system, the method comprising transmitting a carrier signal from the reader device; wherein the carrier signal (a) is received by the transmitter tag and energizes the tag; (b) is altered by the tag to introduce out of band electromotive force (emf) orthogonal to the carrier signal; (c) is then modified by the non-linear impedance element connected to the sensor that measures a condition in an environment in which the transmitter tag is located; (d) is returned to the reader where the carrier signal is filtered from the out of band emf; and (e) is analyzed by the signal processing device.

### 2. Drug Dosing Based on Real Time Measurement of Cancer Treatment Response

In some embodiments, the system comprises an implantable drug delivery device further comprising a cancer monitoring sensor. The system can provide real time information on response to a treatment and up-to-the minute information about the state (growing/shrinking/metastasized) of tumor. The cancer monitoring sensor can detect and monitor one or more of a tumor biomarker, a chemotherapeutic agent, and a tumor metabolite concentration. In some embodiments, the sensor is configured to respond to Her-2/Neu. In some embodiments, the sensor comprises biosensing nanoparticles (e.g., CLIO-anti-hCG-β95 and CLIO-anti-hCG-β97 nanoparticles).

### 3. Drug Dosing Based on Real Time Measurement of Neural Activity

In some embodiments, the system comprises a nanoparticle probe for detecting neural activity, the nanoparticle probe comprising: a core having a substantially spherical shape; a conductive shell disposed over the core; and an electrochromic polymer coating disposed over the conductive shell. The nanoparticle probe can be configured to convert electrophysiological activity to an optically detectable signal that can be picked up from outside the brain using a second window near-infrared (NIR-II, 1000-1700 nm) light reader. Much like the passive radio frequency identification (RFID) tags, the nanoparticle probe can report the spiking activity of cells by modulating the incoming NIR light coupling and the re-radiated light spectrum that is sent back to the reader using backscattering. The spectrum of the backscattered NIR light can be modulated by the electrochromic loading of the plasmonic (electro-plasmonic) nanoantenna, which shows strong sensitivity to the local electric-field dynamics. Thus, each of the nanoparticle probes can provide a bioelectrical signal detection capability in a single nanoparticle device that includes wireless power, electrophysiological signal detection, and data broadcasting capabilities at nanoscale dimensions. US 63/209,49 and U.S. 63/331,409 disclosing nanoprobe for continuous brain monitoring are incorporated herein in their entirety with this application.

### 4. Drug Delivery Cum Stimulation

In an embodiment, an implantable device is a stimulation device. The stimulation device may comprise two functional modules which are, i) a programmable pulse generator module, and ii) a stimulus-receiver module. The stimulus-receiver module is designed to provide stimulation/blocking pulses with an external stimulator. An external device acts as a programmer and as an external stimulator. The system uses an implantable power source until stable external power is available. A power selects circuitry switches between implanted power source and external power source, when its available. Stimulation/blocking to nerve or muscle tissue may be provided using implantable pulse generator, or via an external stimulator which is inductively coupled to the stimulus-receiver portion of the implanted system. Numerous applications of the system include, spinal cord stimulation to provide therapy for intractable pain and refractory angina; occipital nerve stimulation to provide therapy for occipital neuralgia and transformed migraine; afferent vagus nerve modulation to provide therapy for a host of neurological and neuropsychiatric disorders such as epilepsy, depression, Parkinson's disease, bulemia, anxiety/obsessive compulsive disorders, Alzheimer's disease, autism, and neurogenic pain; efferent vagus nerve stimulation for rate control in atrial fibrillation, and to provide therapy for congestive heart failure; gastric nerves or gastric wall stimulation to provide therapy for obesity; sacral nerve stimulation to provide therapy for urinary urge incontinence; deep brain stimulation to provide therapy for Parkinson's disease, and other neurological and neuropsychiatric disorders; cavernous nerve stimulation to provide therapy for erectile dysfunction. US20060074450A1 is incorporated herein in its entirety.

In an embodiment, an implantable device could be to release drug and work as a stimulator simultaneously.

Another application for the systems and method of the current invention is for applying deep brain stimulation (DBS) to subthalamic nucleus or other deep brain structures. Subthalamic nucleus stimulation by means of permanently implanted brain electrodes, is a very effective therapy for all the cardinal features of Parkinson's disease. Generally, such treatment may involve placing a DBS type lead through a burr hole drilled in the patient's skull. Following that, the lead is placed utilizing functional stereotactic brain surgery for applying appropriate stimulation through the lead. The placement portion of the treatment is very critical. The terminal portion of the lead is tunneled to a subcutaneous pocket where it is connected to the pulse generator, which is implanted in a pocket either subcutaneously or sub muscularly.

Another application for the systems and method of this invention is to provide vagal nerve(s) blocking to provide therapy for obesity. The blocking of vagal nerve tissue may be one of DC anodal block, Wedenski block, or collision block. Because of the high frequency of electrical pulses that may be involved for nerve blocking, this application is very demanding on the energy supply of the implanted pulse generator. Advantageously, the system and method of this invention is ideally suited for this type of application.

In an embodiment, the pulsed electrical stimulation/blocking to vagus nerve(s) may be provided using one of the following stimulation systems, such as: a) an implanted stimulus-receiver with an external stimulator; b) an implanted stimulus-receiver comprising a high value capacitor for storing charge, used in conjunction with an external stimulator; c) a programmer-less implantable pulse generator (IPG) which is operable with a magnet; d) a microstimulator; e) a programmable implantable pulse generator; f) a combination implantable device comprising both a stimulus-receiver and a programmable implantable pulse generator (IPG); and g) an implantable pulse generator (IPG) comprising a rechargeable battery. In one embodiment, the external components such as the programmer or external stimulator may comprise a telemetry circuit for networking. The telemetry circuit therefore allows for interrogation or programming of implanted device, from a remote location over a wide area network. U.S. Pat. No. 7,444,184B2 is incorporated herein in its entirety.

### 5. Drug Delivery Cum Device Failure Detector

In another embodiment, the implantable device comprises a second primary sensor that can detect a device failure. The detection of device failure may include categorizing the fault based on the received signal, a clinical context information, or both. The detection of device failure may include categorizing the fault based on the received signal, the clinical context information, or both, and where categorizing the fault includes categorizing the fault as a sensor environment fault or as a system error/artifact fault. The detection of device failure may include categorizing the fault as a sensor environment fault, and further including subcategorizing the fault as a compression fault or an early wound response fault. The detection of device failure may include determining if the received signal or the received data matches or meets a predetermined criterion. The detection of device failure may include analyzing the signal using a time-based technique, a frequency-based technique, or a wavelet-based technique. The detection of device failure may include raw signal analysis, residualized signal analysis, pattern analysis, and/or slow versus fast sampling. The detection of device failure may include projecting the received signal onto a plurality of templates, each template corresponding to a fault mode. The detection of device failure may include variability analysis or fuzzy logic analysis.

6. Drug Delivery with Watchdog Timer Capability

In an embodiment, the implantable drug delivery device comprises a second primary sensor comprising piston position sensor. In some embodiments, the implantable drug delivery device may comprise one or more of a pressure sensor located in the osmotic or pharmaceutical compartments, a real time clock or other method of maintaining relative time regardless of processor reset or exception. In some embodiments, the implantable drug delivery device may comprise a processor with watchdog timer capability. In some embodiments, the implantable drug delivery device may comprise a temperature sensor on components such as the processor, valve, battery, and valve driver circuitry. In some embodiments, implantable drug delivery device may be configured for measuring battery voltage. Further, the implantable drug delivery device may be configured for communicating with a device or devices outside the body in case of abnormal device function.

In some embodiments, the one or more primary sensors are attached to the implantable drug delivery device by providing a pocket for the sensor in the implantable drug delivery device during the manufacturing process. The pocket can be adapted to hold the sensors by utilizing a clamping mechanism. Additionally, or alternatively, the sensor can be secured to the implantable device by an adhesive. The adhesive in this case also serves as a thermal conductor, although water and tissue would perform this task, if the adhesive were not present. The one or more primary sensors can work in continuous mode or in on-demand monitoring mode.

7. Drug Delivery with Telemetry Capability

In some embodiments, the implantable drug delivery device may also comprise a compact multiple-input-multiple-output (MIMO) antenna. The MIMO can be configured for high-data-rate telemetry as well as sensing the inner conditions of the tissues.

In some embodiments a virtual population model can be used to confirm feasibility of certain aspects (such as the thermal aspects and external communications features) of the invention.

In an embodiment, the implantable drug delivery device may be configured to perform a battery level reading function to determine the battery level of the battery. The implantable drug delivery device may be configured to prioritize a task performed by the implantable device based on the battery level reading function such that a non-priority task is not happening simultaneously at the same time as a period of high current demand on the battery. The implantable drug delivery device may be capable of executing concurrently two or more of the functions, and the implantable drug delivery device may be configured to avoid executing concurrently one or more of: (a) a number of functions greater than a maximum number of functions; (b) functions including one or more high current demand functions; and (c) two or more functions that would have a combined current demand higher than a current demand threshold.

### U. System Comprising the Drug Delivery Device and an External Components

### 1. System Comprising the Drug Delivery Device and a Wearable Sensor

An embodiment relates to a system comprising an implantable drug delivery device and an external housing comprising a wearable sensor. In some embodiments, the wearable sensor can measure body vitals to monitor the biochemical changes and biophysical changes. The wearable sensor can comprise one or more of a piezoresistive sensor, a capacitive sensor, an iontronic sensor, a photoplethysmography sensor, a piezoelectric sensor, an electronic sensor, and an optic sensor. The wearable device can be in the form of a tattoo, a dermal patch, a wearable device, a contact lens, a mouthguard, or a wearable fabric. The wearable sensing platform may include a flexible printed circuit board to enable the wearable sensing platform, or a portion thereof, to conform to a portion of the user's body. The wearable sensor can collect one or more of physical activity data, such as step count, distance traveled, and calories burned, heart rate data, including resting heart rate and heart rate variability, sleep data, such as sleep duration, sleep quality, and the number of times the user wakes up during the night, blood pressure data, including systolic and diastolic readings, oxygen saturation (SpO2) data, which measures the amount of oxygen in the blood, temperature data, such as body temperature and ambient temperature, respiration data, including breathing rate and patterns, electrocardiogram (ECG) data, which measures the electrical activity of the heart, electrodermal activity (EDA) data, which measures changes in the conductivity of the skin in response to emotional or physical stress, blood glucose data, which measures the level of glucose in the blood and is commonly used by people with diabetes.

In some embodiments, the wearable sensor may also indicate the start of a disease. If the wearable sensor monitors the start of a disease, it can send a signal to a remote computing device of patient and/or doctor to remove the implantable device. In an example, the wearable sensor senses aspects of a user's state by analyzing bodily fluids, such as sweat and/or urine, and a user's temperature and indicates start of a kidney related disease. A sensor array senses a plurality of different body fluid analytes, optionally at the same time. A signal conditioner is coupled to the sensor array. The signal conditioner conditions sensor signals. An interface is configured to transmit information corresponding to the conditioned sensor signals to the remote computing device. A system of this kind described in US20180263539A1 is incorporated herein by reference in its entirety.

In some embodiments, the system comprises a wearable EEG sensor configured to monitor electroencephalography signals and an implantable drug delivery device configured for delivering a seizures drug (e.g. benzodiazepines, such as diazepam, lorazepam, or midazolam) wherein the drug delivery is configured for triggering subcutaneous drug release through wireless voltage induction.

### 2. System Comprising the Drug Delivery Device and an Implantable Sensor

An embodiment relates to a system comprising an implantable device and a secondary sensor comprising an implantable sensor. In some embodiments, the implantable sensor can measure body vitals to monitor the start of a disease such as a kidney disease or a heart related disease. The system can calculate an analyte level and an analyte level rate of change using at least measurement information conveyed by the implantable sensor. If the sensor monitors the start of a disease, it can send a signal to the remote computing device of patient and/or doctor to remove the implantable device.

### 2.1 System Comprising the Drug Delivery Device and a Creatinine Sensor

In an example, the system comprises an implantable drug delivery device and an implantable creatinine sensor. Creatinine is an important clinical analyte for monitoring heart failure patients. The system can include an external monitoring device and a chemical sensor in communication with the external monitoring device. The chemical sensor can be configured to detect creatinine concentration in a bodily fluid. The chemical sensor can include a sensing element comprising creatinine deiminase covalently bound to a substrate and a pH-indicating compound in ionic communication with the creatinine deiminase. A system of this kind described in US20090124875A1 is incorporated herein by reference in its entirety.

### 2.2 System Comprising the Drug Delivery Device and a PAP Sensor

In another example, the system comprises an implantable drug delivery device to deliver drugs such as diuretics and an implantable pulmonary artery pressure (PAP) sensor that senses a PAP signal, and the implantable medical device detects hypovolemia from the PAP signal. A system of this kind described in U.S. Pat. No. 8,414,497B2 is incorporated herein by reference in its entirety.

The delivery of diuretics may be modulated or completely stopped based on the information obtained from the PAP signals.

### 3. System Comprising the Drug Delivery Device and a Wireless Charging Component.

An embodiment relates to a system comprising an implantable drug delivery device and an external housing comprising an external charging circuit for wirelessly recharging the battery of the implantable drug delivery device. In an example, the implantable drug delivery device comprises a secondary coil capable of receiving energy from the external charging circuit. A magnetically shielding material is positioned between the secondary coil and a drug delivery unit of the implantable drug delivery device. A primary coil carried in the external housing, the primary coil being capable of inductively energizing the secondary coil when the housing is externally placed in proximity of the secondary coil with a first surface of the housing positioned closest to the secondary coil, the first surface of the housing being a thermally conductive surface. An energy absorptive material (e.g., wax) is carried within the external housing. The energy received for charging does not affect the health of the patient.

An embodiment relates to a system comprising an implantable device and an external housing comprising a transceiver capable of transmitting one or more transmission signals. In some embodiments, the transceiver may communicate with and power the implantable device. The transceiver may receive one or more transmission signals from the implantable device and may calculate one or more information based on the one or more transmission signals. The transceiver may generate a measurement trend, alerts, and/or alarms based on the calculated one or more information's. The system may also include a display device, which may be, for example, a smartphone and may be used to display analyte measurements received from the transceiver. The display device may execute a mobile medical application. The system may include a data management system, which may be web-based. The one or more transmission signal comprises at least power or data.

### 4. System Comprising the Drug Delivery Device and a Transmission Component.

In some embodiments, the drug delivery implantable device is configured to receive the one or more transmission signals from the external housing and the external housing comprises a first external device comprising at least one external antenna configured to transmit a first transmission signal to the implantable drug delivery device, the first transmission signal comprising at least power or data. The transceiver may be configured to (i) receive measurement information from the primary sensor and/or secondary sensor, (ii) establish a connection with a display device while being connected with no other device, (iii) convey the measurement information to the display device while connected with the display device, (iv) establish a connection with a second device while being connected with no other device, and (v) convey or receive second information to or from the second device while connected with the second device. In some embodiments, the transceiver operating according to the clinical mode may (i) generate one or more alerts, alarms, and notifications and (ii) convey some but not all of the generated alerts, alarms, or notifications to the display device. In some embodiments, the generated but not conveyed alerts, alarms, or notifications may include analyte-related alerts, alarms, or notifications. In some embodiments, the conveyed alerts, alarms, or notifications may include analyte-unrelated alerts, alarms, or notifications. In some embodiments, the conveyed alerts, alarms, or notifications may include one or more calibration notifications and/or one or more transceiver battery level notifications. In some embodiments, the transceiver may implement a passive telemetry for communicating with the implantable device via an inductive magnetic link for both power and data transfer. The link may be a co-planar, near field communication telemetry link. The transceiver may include a reflection plate configured to focus flux lines linking the transceiver and the sensor uniformly beneath the transceiver. The transceiver may include an amplifier configured to amplify battery power and provide radio frequency (RF) power to a transceiver antenna. A system of this kind described in US20220126103A1, U.S. Pat. No. 9,867,540B2, U.S. Ser. No. 11/116,402B2, USD853382B1 are incorporated herein by reference in its entirety.

### 5. System Comprising the Drug Delivery Device and a Thermal Regulation Component.

An embodiment relates to a system comprising an implantable device and an external housing comprising a thermoregulator capable of thermo-heating or cooling of the implantable device to maintain the implantable device at the body temperature of around 37-38° C. even if the body temperature fluctuates. The thermoregulator may include a power negotiation protocol. This protocol may be hardware, firmware and/or software, and may regulate charger function, including switching the charger on or off (including altering the duty cycle of the charger). The system of this kind described in U.S. Ser. No. 11/318,250B2 is incorporated herein by reference in its entirety.

### V. Multi-Component System

An embodiment relates to a system comprising an implantable drug delivery device, an implantable sensor and the external housing having one or more of an external charging circuit for wirelessly recharging the battery of the implantable device, a transceiver capable of communication with an implanted sensor and external devices such as a smartphone, a thermoregulator capable of thermo-heating or cooling of the implantable device to maintain the implantable device at the body temperature, and a wearable sensor. The system can be equipped with one or more primary sensors and/or one or more secondary sensors to monitor biochemical and/or biophysical changes. A wireless transceiver of the system can receive instructions through radio waves from an external device and transmit the instruction to the implantable drug delivery device. The system has capability to monitor biological parameters and dispense drugs as per a command either generated by the microprocessor in the device or a command received via an external device.

In an embodiment, the system herein can utilize a closed-loop feedback control method to ensure accurate drug delivery. The implantable drug delivery device may include a closed loop unit that includes the one or more primary sensors (and information/output sensed by them) and a valve actuator configured for drug delivery. In some embodiments, the implantable drug delivery device is guided by the artificial intelligence system for feedback control as described in US20140088554A1 which is incorporated with this specification in its entirety. This allows output from the one or more sensors to be used to directly modulate or control the rate of drug delivery from the drug delivery orifice, including stopping drug delivery altogether. The system is adapted to perform one or more of the following: collect the data from the one or more primary sensors, combine it with other diagnostic data that may have been gathered by a caregiver, perform a deep analysis of the disease state of the patient, arrive at a diagnosis, and decide whether to dynamically alter the drug delivery rate based on this diagnosis. The results of the computerized analysis of the disease state and the subsequent diagnosis can be reviewed by the caregiver and altered, modified, or rejected altogether. In these feedback loops, the caregiver can affect and control the operation of the system either through direct intervention at the point when the trigger to alter the state of the device is about to be reset, or at the point where the analysis of the disease state has been made by one or more computer executable methods that preferably utilize artificial intelligence to complete the analysis.

In an example, the system comprises implantable drug delivery device configured to release an anti-diabetic drug and a blood glucose monitoring sensor. The blood glucose sensor monitors the indicators of disease/symptoms and regulates the release of a drug to help treat the disease.

In an embodiment, the system comprises a soft robotic implant with sensor and actuator arrays. The soft robotic implant may be made from soft, flexible materials that can be designed to conform to the shape of the body. In some embodiments, the device can be powered by small, lightweight motors and controlled by computer algorithms. Because they are made from soft materials, they are less likely to cause irritation or damage to the surrounding tissues than traditional implants made from hard, rigid materials. The implantable device is more adaptable to changes in the body's shape and movement, making them more comfortable for the subject and less likely to become dislodged or malfunction. In some embodiments, the implantable device can be programmed to respond to signals from the subject's nervous system, allowing for more natural and intuitive control of the device.

### W. Multiple Drug Delivery

In an embodiment, the device is configured to deliver more than one drug. The device comprises plurality of osmotic units, plurality of drug reservoir units, plurality of drug delivery unit and one or more electronic unit. The plurality of drug reservoir units comprise plurality of drugs. These drugs can be delivered simultaneously. In some embodiments, the device can be programmed to deliver the plurality of drugs in pre-defined intervals. In some embodiments, the device can deliver one or more of the plurality of drugs based on the physiological conditions sensed by the sensors. In some embodiments, the device can be regulated to deliver the drugs from outside the subject's body via the implantable device inside the body.

### Valve Based Multiple Drug Delivery Implantable Device

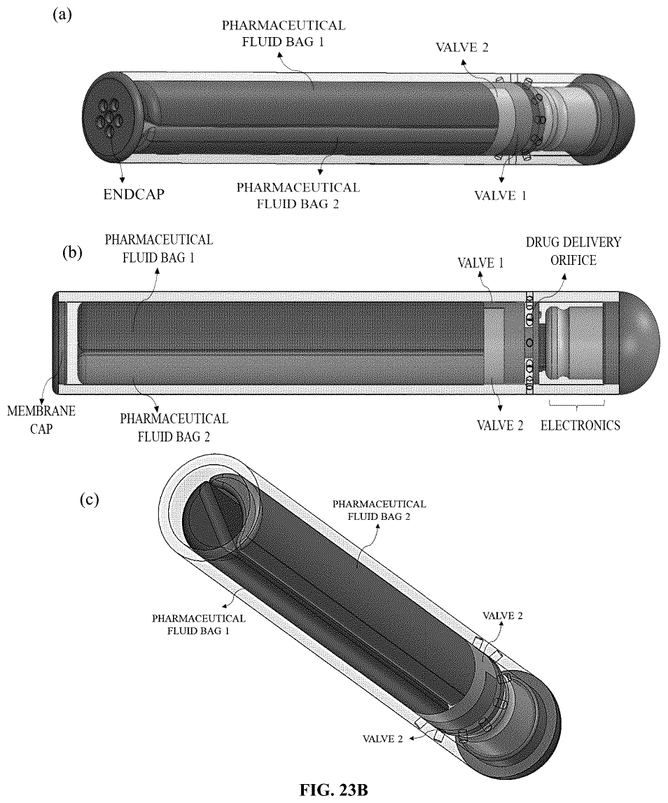

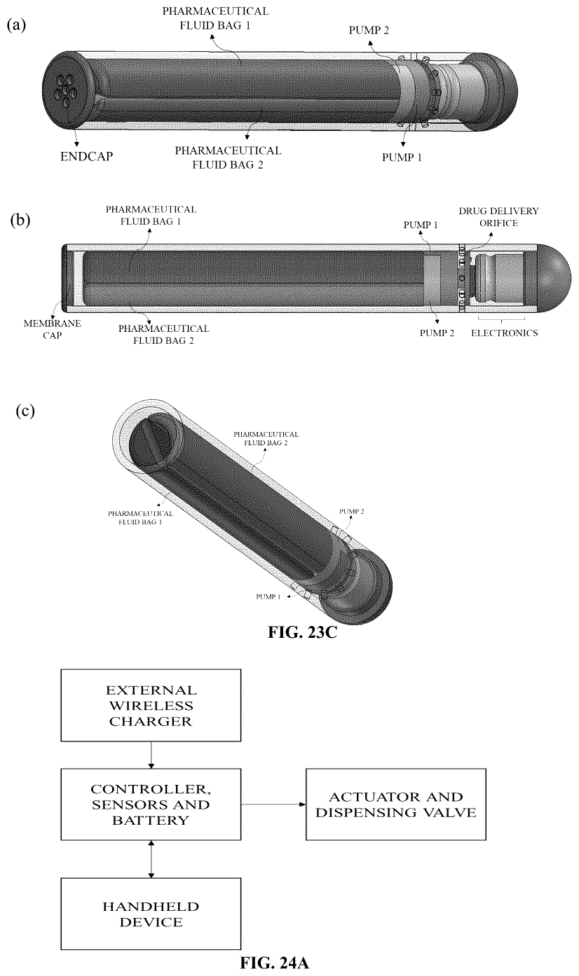

Referring to FIG. 23A, the device comprises plurality of osmotic units, plurality of drug reservoir unit, plurality of drug delivery unit and one or more electronic unit. The plurality of drug reservoir unit comprises a plurality of drugs. In an example, one of the drug reservoir units may have Metformin and the other drug reservoir unit may comprise GLP-1 receptor agonists. In another example, one of the drug reservoir units may have Metformin and the other drug reservoir unit may comprise repaglinide receptor agonists. The examples provided here are non-limiting and many other such drug combinations may be delivered using the device.

In some embodiments, the drug combination is such that one of the drugs reduces the adverse effects of another drug.

In some embodiments the device comprises multiple pharmaceutical bags fluidically connected to multiple valves. The valves are electronically controlled to deliver the drugs either simultaneously or to deliver a first drug D₁ at a time t₁ and a second drug D₂ at a time t₂.

Referring to (a) of FIG. 23B, it shows external view of multi-drug multi-valve device uses osmotic pressure to apply pressure to the internal pharmaceutical filled bags allowing the liquid drug to flow into the body when the respective valve is open. The osmotic solute is located in the endcap either near or on the osmotic membrane. The endcap also comprises a semi permeable membrane. Referring to (b) of FIG. 23B, it shows an internal view of multi-drug multi-valve device, according to one or more embodiments. Two or more long flexible and compressible bags of pharmaceutic fluid are attached to the walls of the tube in the drug reservoir chamber. The pharmaceutical fluid bag 1 connects fluidically to valve 1 and pharmaceutical fluid bag 2 connects fluidically to valve 2. Interstitial Fluid enters via the membrane cap through the osmotic membrane enters into osmotic chamber pressurizing the pharmaceutical bags. Electronics and battery control and power the two valves. The valves for (example a piezoelectric valve or a combination of valves described somewhere else in this application) release the pharmaceutic fluid (drug) from pharmaceutical drug reservoir chamber out to body. Referring to (c) of FIG. 23B, it shows a side-view of internal arrangement in the multi-drug multi-valve device, according to one or more embodiments. Multiple bag/valve sets can be used (1 or more). Each of the pharmaceutical bags can be dosed independently. (c) of FIG. 23B shows 2 independent bag/valve sets. All bag/valve sets may be powered and controlled by a single processor and battery. In (c) of FIG. 23B, dosing of pharmaceutical 1 has not occurred so the bag is full. However, pharmaceutical 2 has been partially dosed through Valve 2, so the bag has partially collapsed against the tube wall. The bags are attached to the wall to prevent drifting.

### Pump Based Multiple Drug Delivery Implantable Device

In some embodiments, the drug reservoir unit may comprise a plurality of drug reservoir bags and/or plurality of pump sets that can be used for delivery of two or more drugs simultaneously or in a pre-defined time period. Each of the drug reservoir bags can be dosed independently. All bag/pump sets may be powered and controlled by a single processor and battery. Each drug reservoir bag is connected to a corresponding pump. Each bag may be partially adhered to the inside walls of the tube to prevent twisting of the bag as the bag is drained. Interstitial fluid enters through holes in the endcap to fill the void left as the bags are drained. Each pump may be activated individually.

In an embodiment, the device is configured to deliver more than one drug. The device comprises plurality of osmotic units, plurality of drug reservoir units, plurality of drug delivery unit and one or more electronic unit. The plurality of drug reservoir units comprise plurality of drugs. These drugs can be delivered simultaneously. In some embodiments, the device can be programmed to deliver the plurality of drugs in pre-defined intervals. In some embodiments, the device can deliver one or more of the plurality of drugs based on the physiological conditions sensed by the sensors. In some embodiments, the device can be regulated to deliver the drugs from outside the subject's body via the implantable device inside the body.

In an example, the device comprises plurality of osmotic units, plurality of drug reservoir unit, plurality of drug delivery unit and one or more electronic unit. The plurality of drug reservoir unit comprises a plurality of drugs. In an example, one of the drug reservoir units may have Metformin and the other drug reservoir unit may comprise GLP-1 receptor agonists. In another example, one of the drug reservoir units may have Metformin and the other drug reservoir unit may comprise repaglinide receptor agonists. The examples provided here are non-limiting and many other such drug combinations may be delivered using the device.

In some embodiments, the drug combination is such that one of the drugs reduces the adverse effects of another drug.

In some embodiments the device comprises multiple pharmaceutical bags fluidically connected to multiple pumps. The pumps are electronically controlled to deliver the drugs either simultaneously or to deliver a first drug D₁ at a time t₁ and a second drug D₂ at a time t₂.

Referring to (a) of FIG. 23C, it shows external view of multi-drug multi-pump device uses osmotic pressure to apply pressure to the internal pharmaceutical filled bags allowing the liquid drug to flow into the body when the respective pump is open. The osmotic solute is located in the endcap either near or on the osmotic membrane. The endcap also comprises a semi permeable membrane. Referring to (b) of FIG. 23C, it shows an internal view of multi-drug multi-pump device, according to one or more embodiments. Two or more long flexible and compressible bags of pharmaceutic fluid are attached to the walls of the tube in the drug reservoir chamber. The pharmaceutical fluid bag 1 connects fluidically to pump 1 and pharmaceutical fluid bag 2 connects fluidically to pump 2. Interstitial Fluid enters via the membrane cap through the osmotic membrane enters into osmotic chamber pressurizing the pharmaceutical bags. Electronics and battery control and power the two pumps. The pumps for (example a piezoelectric pump or a combination of pumps described somewhere else in this application) release the pharmaceutic fluid (drug) from pharmaceutical drug reservoir chamber out to body. Referring to (c) of FIG. 23C, it shows a side-view of internal arrangement in the multi-drug multi-pump device, according to one or more embodiments. Multiple bag/pump sets can be used (1 or more). Each of the pharmaceutical bags can be dosed independently. (c) of FIG. 23C shows 2 independent bag/pump sets. All bag/pump sets may be powered and controlled by a single processor and battery. In (c) of FIG. 23C, dosing of pharmaceutical 1 has not occurred so the bag is full. However, pharmaceutical 2 has been partially dosed through Pump 2, so the bag has partially collapsed against the tube wall. The bags are attached to the wall to prevent drifting.

### X. Electronics and Electronic Systems for the Drug Delivery Device

In an embodiment, the implantable device comprises a miniaturized and highly integrated electronic system. In some embodiments, the electronic system may comprise an electrically coupled processor, memory, a power source, and a communication component. In some embodiments, an electronic system may comprise a wireless controller. In some embodiments, an electronic system may comprise a RF communication component. In some embodiments, an electronic system may comprise blue-tooth technology. A controller may be contained within the unit that is physically connected to a pump (e.g., a catheter) or it may be spaced away and/or operate remotely in some embodiments. A controller may be contained, for example, in a wrist watch and/or a mobile communication device (e.g., a cell phone). In some embodiments, sensor data from one or more sensors in the implantable device is processed by a central microcontroller unit (MCU) which analyzes the information and controls the actuator. This actuator, powered by a reliable and biocompatible battery or energy harvesting source, could be a flow switch for drug delivery, controlled with precise electrical signals. Crucial for data transmission and external interaction is the communication component, often utilizing low-power wireless protocols like Bluetooth or near-field communication (NFC). The entire system is encased in biocompatible materials, ensuring safety and long-term functionality within the body.

In an embodiment, the implantable drug delivery device includes a wireless charger for power transfer without physical connectors, enhancing usability and safety. In some embodiments, a rechargeable battery is integrated, designed to withstand physiological conditions. The energy management system ensures optimal power usage, contributing to sustainability. In some embodiments, the device includes an actuator driver interpreting signals from the microcontroller for controlled drug delivery.

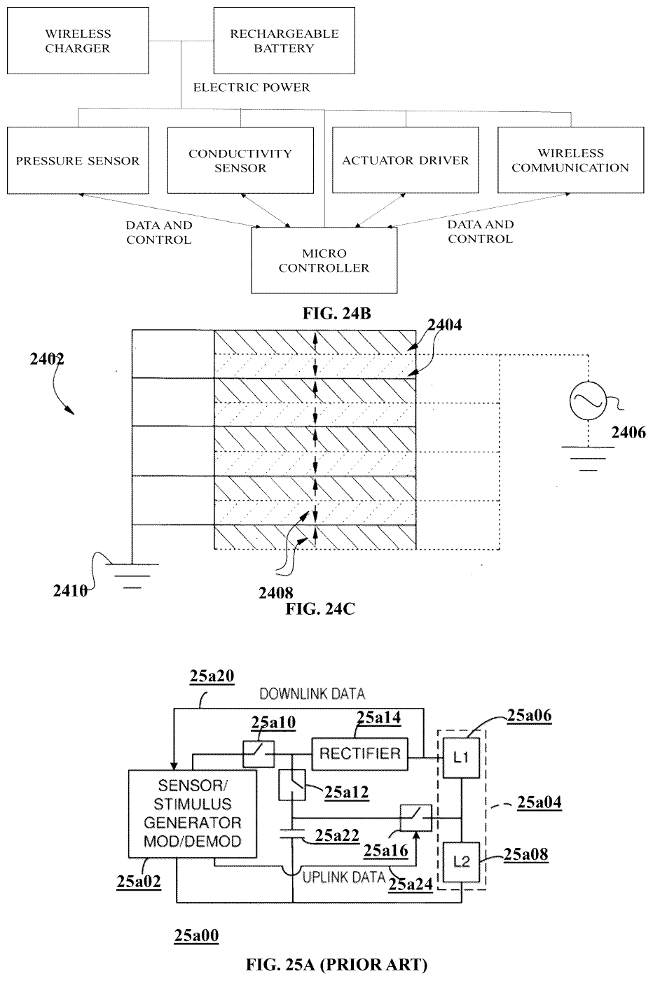

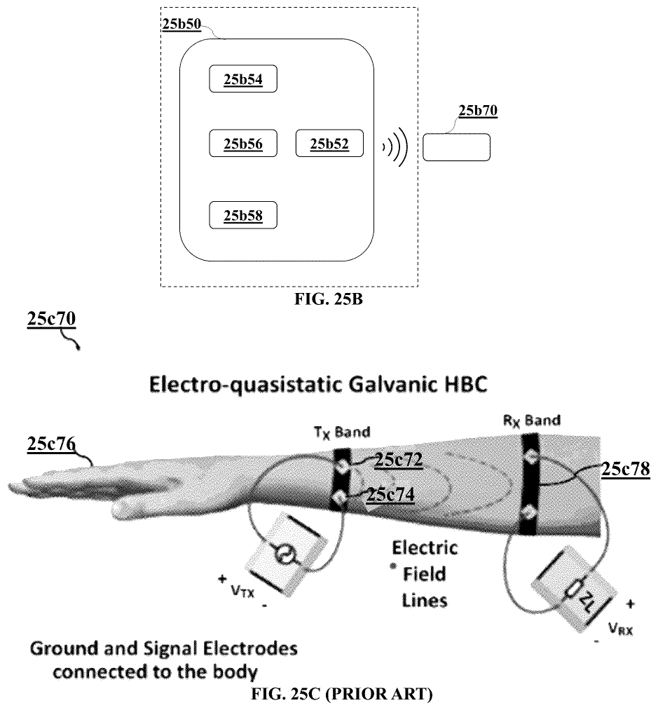

The device electronics provide reliable and precise dosage administration. FIG. 24A shows a schematic of electronic components of the implantable device, according to one or more embodiments.

In an embodiment, the pressure sensor provides real-time feedback to the microcontroller, enabling adaptive drug delivery to optimize dosage administration and minimize complications.

In some embodiments, the device includes a conductivity sensor, monitoring osmotic agent conductance, providing real-time feedback to the microcontroller. This sensor data allows drug delivery precision by responding to variations in conductivity levels, aligning the administered dosage with the patient's needs.

In an embodiment, the implantable drug delivery device is configured for wireless communication with external devices for remote monitoring and control. Healthcare professionals can adjust settings, monitor patient compliance, and receive real-time health data, improving overall device management.

In an embodiment, the control module of the implantable drug delivery device integrates essential components to ensure optimal functionality and adaptability. FIG. 24B shows a schematic of a control module of an implantable device, according to one or more embodiments. The control module's integration of two or more of a wireless charger, rechargeable battery, actuator driver, pressure sensor, conductivity sensor, wireless communication, and microcontroller establishes a comprehensive and advanced platform for precise, adaptable, and patient-specific drug administration within the implantable drug delivery device.

### Y. Alternative Embodiments for Drug Delivery Device

Piezoelectric materials have garnered significant attention in recent years due to their unique ability to convert mechanical energy into electrical energy and vice versa. This property makes them ideal for various applications, and one area where they hold great promise is drug delivery systems. In this article, we will delve into the world of piezoelectric motion and explore its potential applications in drug delivery. We will also discuss alternative embodiments that could revolutionize the way drugs are administered.

Piezoelectric materials generate an electrical charge when subjected to mechanical stress and, conversely, deform in response to an applied electrical field. This unique characteristic opens up a world of possibilities for motion control, particularly in drug delivery systems. Various types of piezoelectric motion, such as stacked piezo elements for linear movement, flexing arms (bimorph benders), and discs, offer different advantages for specific applications.

### Applications of Piezoelectric Motion in Drug Delivery:

Piezo Walkers and Stick Slip Piezos: Piezo walkers and stick slip piezos show immense potential in moving shuttles for drug delivery systems. Their precise control and high responsiveness make them ideal for navigating through intricate pathways within the body, delivering medication to targeted areas.

Disc Actuator for Pump and Valve Functions: The disc actuator, when flexed concave and convex based on voltage, can effectively serve as both a pump and a valve. The pump function propels pharmaceutical fluids, while the valve function controls the flow, enabling accurate dosage control.

Bimorph Benders as Flapper Valves: Bimorph benders, with their flexing arms, can effectively act as flapper valves. This motion can regulate fluid flow and ensure that medications are delivered with precision.

Ovoid or Flying Saucer-Shaped Flexible Pharmaceutical Enclosures: Innovative designs that incorporate flexible circuit boards containing pumps can attach to ovoid or flying saucer-shaped bags. These flexible enclosures can exert pressure on the fluid, allowing the use of valves instead of pumps for drug delivery.

In an embodiment, the osmotic pump can be replaced by other pressurizing mechanism, such as a spring, or a pressurized gas or liquid system, such as carbonated water.

Squat Can with Internal Bladder: A unique embodiment involves a flat rigid disc with an internal bladder, similar to an accumulator. The disc can accordion to generate pressure, or ambient pressure can be utilized alongside a pump for fluid movement.

Spring Instead of Osmotic Pressure: The application of a spring, in place of osmotic pressure, provides an alternative mechanism for drug delivery. This approach can be adapted to various piezoelectric motion designs for more precise control.

Magnetohydrodynamic (MHD) Pumps: While magnetohydrodynamic pumps provide low-pressure, non-positive displacement fluid movement, their small size embodiments pose challenges with fluid flow. However, they have the potential for extremely low energy demands and no moving parts, making them an exciting prospect for future research and development.

Piezoelectric motion holds significant promise for revolutionizing drug delivery systems. Its precise control, energy efficiency, and adaptability make it an ideal candidate for targeted drug administration. The exploration of alternative embodiments adds further depth to this potential, offering innovative solutions that could shape the future of pharmaceutical delivery.

The utilization of piezoelectric motion in drug delivery systems represents a groundbreaking advancement that holds great potential in improving patient outcomes and revolutionizing healthcare. With further research and development, piezoelectric motion can become a cornerstone in the advancement of drug delivery technologies.

Referring to FIG. 24C, it shows a diagram of a piezoelectric actuator stack 2402. In various embodiments, the piezoelectric actuator stack 2402 corresponds to the single crystal piezoelectric actuator stack. The actuator stack 2402 includes layers of a piezoelectric material 2404. Each layer of the piezoelectric material 2404 is made from a single crystal of piezoelectric material. Each layer of piezoelectric material 2404 has a poling direction 2408, a voltage source 2406 and an electrical ground 2410. The layers of piezoelectric materials 2402 are identical or similar sizes and are stacked on top of each other. The layers of piezoelectric materials 2402 are also connected to both voltage source 2404 and electrical ground 2410.

In an embodiment of the valve-based device, comprises a piezoelectric material stack, said piezoelectric material stack comprising one or more layers of piezoelectric single crystals. When a voltage is introduced to the stacked piezo actuator, it induces a strain or displacement aligned with the polarization. The displacement of a piezoelectric element is determined by the voltage magnitude multiplied by the piezoelectric coefficient. The small displacement caused by the stacked piezo actuator serves as a means to generate pressure, which in turn facilitates the opening of a valve and the delivery of a substance within the subject's body.

In an embodiment of the valve-based device, it comprises stacked piezo actuator. These benders are constructed of multiple, co-fired piezoceramic layers, in which the voltage bias across the top half of the layers is controlled independently of the voltage bias across the bottom half of the layers. The length of the layers increases as the voltage applied across them increases. Tip deflection of the bender occurs when the top and bottom sets of layers elongate by different amounts. For example, downward displacement of the bimorph bender occurs when the top layer elongates while the length of the bottom layer remains the same.

In an embodiment, a piezoelectric stack will be equipped with two strain gauges, which will be the photograph of an actuator. When the strain gauges are connected in a half Wheatstone bridge, the deformation of the piezoelectric stack can be monitored, and the block will function as both an actuator and a sensor.

In an embodiment, the device comprises piezoelectric actuator that is configured for aiding in pumping of the drug to the subject. The piezoelectric actuator is based on the piezoelectric effect and converts electrical energy directly into linear motion with virtually unlimited resolution. It provides efficient force generation because no conversion from rotary to linear motion and no gears or mechanical other parts that require maintenance or lubrication are involved. The non limiting examples of basic motions of piezoelectric elements are stacked piezo elements, flexing arm (bimorph benders), piezo disc, piezo walkers, and stick slip type piezo. In an example, piezo walkers and stick slip piezo can be used for moving a shuttle. In another example, a disc actuator may lend itself to a pump and a valve. In yet another example, a bimorph bender may work as a flapper valve. In yet another example, a piezo stack may be useful for opening a valve.

Multiple piezoelectric elements can be stacked on top of one another, forming what is referred to as a stacked piezo actuator. These devices harness the collective expansion of each element to generate a beneficial motion and force. Each individual piezoelectric element in a stacked actuator possesses alternating polarity, and when electrical field is applied parallel to the polarization direction the stacked actuator converts an electrical signal into a precisely controlled physical displacement.

### Z. Power Source/s for the Device

In an embodiment, the power source in the device is a primary battery. In an embodiment, the power source for the device is a primary battery. The primary battery can be a lithium-based battery, zinc-mercury based battery, or bioresorbable zinc primary batteries. Examples of primary lithium-based batteries are lithium iodine batteries, lithium thionyl chloride batteries, lithium silver vanadium oxide batteries, lithium carbon mono fluoride batteries, and lithium manganese oxide batteries.

In another embodiment, the power supply is given by an external source or with rechargeable batteries that can be recharged periodically, using the external source. Different technologies that can be used to implement wireless power transfers: near field resonant inductive coupling, near field capacitive coupling, ultrasound based wireless systems, solar power harvesting electromagnetic midfield-based systems and electromagnetic field-based systems.

US 2014/0055088 describes a method for wireless charging of implant devices. A backscatter from the communication coil is used to indicate the best frequency for efficient charging. Thus, the external transmitter can adjust the frequency for wireless power transmission based on a basic feedback mechanism using backscattered information.

US 2014/0084855 describes wireless power transmission and data transmission to the implant. The backscatter signal is received by an external system and processed to control the implant impedance matching unit or to change the frequency of the external device. In both cases, this is done to obtain maximum wireless power transfer.

The energy transfer using inductive coupling comprises the placement of two coils positioned in close proximity to each other on opposite sides of the cutaneous boundary. The internal coil, or secondary coil, is part of or otherwise electrically associated with the implanted medical device. The external coil, or primary coil, is associated with the external power source or external charger, or recharger. The primary coil is driven with an alternating current. A current is induced in the secondary coil through inductive coupling. This current can then be used to power the implanted medical device or to charge, or recharge, an internal power source in the device, or a combination of the two.

In some embodiments, the implantable device may be recharged by magnetic induction. In an example, the implanted device may be operated on a single rechargeable cell, or a dual power source system, and the rechargeable complement may be recharged by magnetic induction as described in U.S. Pat. No. 5,411,537 included as incorporation by reference in its entirety.

In some embodiments, a current with a sinusoidal waveform is applied to a resonant circuit comprising a primary coil and a capacitor. Current is induced in a secondary coil attached to the implanted medical device. Two solid-state switches are used to generate the sinusoidal waveform by alternately switching on and off input voltage to the resonant circuit. The batteries are charged using a charging protocol that reduces charging current as the charge level in the battery decreases.

An alignment indicator is also provided to ensure proper alignment between the energy transmission device and the implanted medical device. This power transfer technique is described in U.S. Pat. No. 5,690,693, included in this specification as incorporation by reference in its entirety. Similar technologies are described in U.S. Pat. Nos. 6,324,430, 6,505,077, 5,411,537, 5,690,693, 6,324,430, and 6,505,077, included in this application as incorporation by reference in their entirety.

In an embodiment, the implants include a single tapped coil antenna having a first section and a second section for wireless power transfer, data downlink and data uplink. A modulated wireless power transfer signal is received by the tapped coil antenna and used to provide electrical power. The modulation is detected to generate downlink data. A switch is used to charge a section of the tapped coil antenna by establishing a current which is then interrupted by opening the switch. The switching produces a high amplitude pulsed magnetic field (PMF) for use in data uplink over a large distance between the implant and an external transceiver.

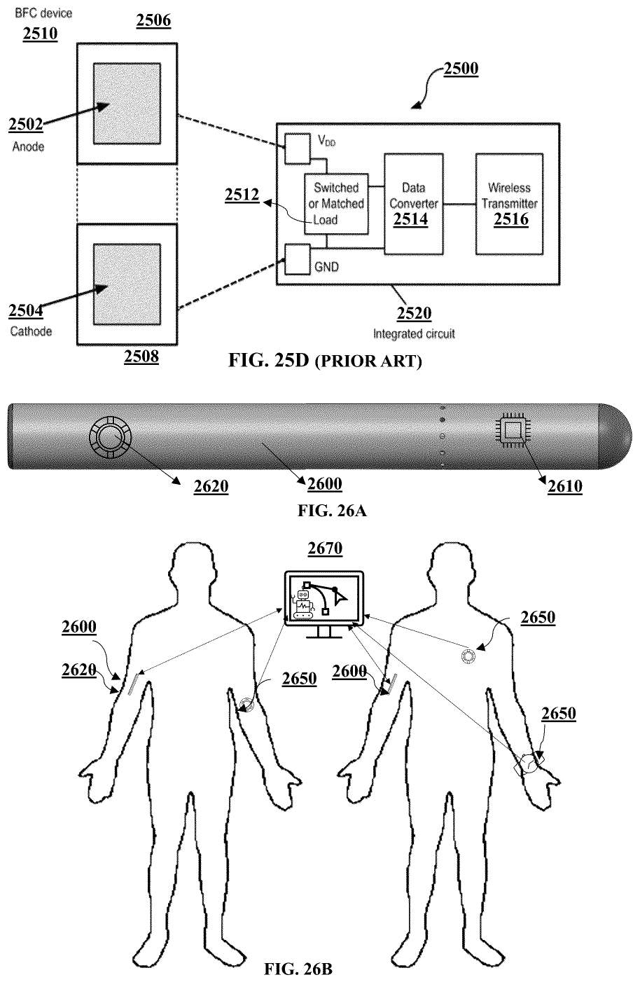

Referring to FIG. 25A, a representative implant system 25a00 comprising an implant device 25a02 that includes electronics for one or more of sensing, stimulus generation, data modulation and demodulation, power management, and other functions. The device 25a02 is coupled to a tapped coil antenna 25a04 that includes a first section 25a06 and a second section 25a08. The device 25a02 is also coupled to switches 25a10, 25a12 that are operable to control wireless power transfer (WPT) based on a received electrical signal from the tapped antenna 25a04 that is produced in response to an RF signal. Downlink data from the tapped coil antenna 25a04 is directed to the implant device 25a02 along a circuit path 25a20 for demodulation. In one example, the downlink data is transmitted as amplitude-shift-keying (ASK) data. During time intervals associated with WPT the switches 25a10, 25a12 can be closed so that the rectified received electrical signal is coupled to charge capacitor 25a22 and power the implant device 25a02. During WPT, downlink data can be obtained from a demodulation of the received electrical signal. For example, an RF signal at a fixed frequency can be used for power transfer and this RF signal can be amplitude modulated to provide downlink data. Thus, a common RF signal can be used for WPT and downlink communication. The switches 25a10, 25a12 can be used to decouple some circuit portions as needed. Uplink data is provided by a modulator associated with the implant device 25a02 to a switch 25a16. Upon receipt of uplink data along path 25a24, the switch 25a16 can be selectively toggled (open to closed or vice versa) to generate a pulsed magnetic field. By controlling the switch 25a16 based on the uplink data, a series of magnetic field pulses is produced that can be transmitted to a remote receiver. The capacitance of the capacitor 25a22 and the inductances of the tapped coil antenna 25a04 are generally selected so that the magnetic field pulses are associated with oscillations at a frequency corresponding to frequencies used for WPT and downlink communication. Signals applied to the second section 25a08 induce larger signals in the tapped coil antenna 25a04 (both sections) so that the pulsed magnetic fields (PMFs) emitted can have larger amplitudes.

In another embodiment, a wireless power transfer system comprises a driver coil array, a hexagonally packed transmitter mat, a receiver coil, and a load coil for powering a medical implant. The magnetically coupled resonance between two isolated parts is established by an array of primary coils and a single small secondary coil to create a transcutaneous power link for implanted devices as moving targets. The primary isolated part comprises a driver coil array magnetically coupled to a mat of hexagonally packed primary coils. Power is injected by the driver coils into the transmitter coils in the transmitter mat to maintain resonance in the presence of losses and power drawn by the receiver coil from the magnetic field. The implanted secondary isolated part comprises a receiver coil magnetically coupled to a load coil. A rectification/filter system is connected to the load coil supplying DC power to the electronic circuits of the implant. Each driver coil-primary coil pair forms a voltage step-up transformer to produce a strong resonance for wireless power delivery to the secondary coils implanted within the human or animal body.

Referring to FIG. 25B, it shows a schematic of an implant adapted to receive wireless power from an external transceiver 25b70 via an ultrasound signal. The implant 25b50 is placed within a patient's body (which could be a human or any animal).

In certain embodiments, an external transceiver 25b70 activates the circuitry of the implant 25b50 by transmitting an ultrasound signal to the ultrasonic transducer 25b50. The ultrasonic waves from the signal are received by the ultrasonic transducer and converted into electrical energy. The transducer can be made of materials like piezoelectric polyvinylidene fluoride (PVDF) flexible thin film, which is operably connected to another circuitry within the implant. A controller 25b54 is programmed such that when it receives ultrasound waves corresponding to a modulated signal, it will close an electrical switch and activate the implant. Conversely, in other embodiments, a specific step function may be used to open an electrical switch and deactivate the implant to conserve power stored in a power storage device 25b56. The controller 25b54 may be programmed to time out the implant after a certain period. For instance, if the ultrasound transducer 25b52 has not sent or received an ultrasound signal during a test period, the controller 25b54 will deactivate the implant. To conserve power, the controller 25b54 can: a) put the implant 25b50 to sleep for a specific duration, b) during the “awake” period, perform tasks like transmitting ultrasound signals, obtain measurements using plurality of sensors 25b58, control an actuator, or communicate with other electronics, c) enter the “asleep” state.

In an embodiment, the actuator coil in a flow switch may serve a dual purpose by not only facilitating the actuation of the spring mechanism for flow control but also providing a solution for charging batteries. When the actuator coil is not actively engaged in the actuation process, it can be repurposed to generate electrical energy through electromagnetic induction. This surplus energy can then be harnessed for charging batteries, offering a sustainable and efficient power source. This integrated approach enhances the overall functionality of the flow switch, transforming it into a versatile component that not only ensures precise flow control but also contributes to energy harvesting for powering associated electronic systems or external devices.

In an embodiment, in idle state, the actuator coil is not energized, and the mechanical spring is relaxed. The flow switch remains in its default position, obstructing the drug flow. In the charging mode, the actuator coil can be connected to a rectifier circuit. The rectifier converts the alternating current (AC) induced in the coil (due to nearby magnetic fields) into direct current (DC). This DC current can be used to charge a battery connected to the circuit.

In some embodiments, as the drug flows out of the device, the actuator coil experiences magnetic induction. The induced AC voltage generates a small current, which is rectified and used to charge the battery. The battery stores this energy for later use.

In an embodiment, the rechargeable battery in the implantable device is continuously charged by a wearable device which has a large capacity rechargeable battery. In another embodiment, the rechargeable battery of the wearable (device) is charged either wirelessly or via a normal charger for wearables (like iWatch). In yet another embodiment, the wearable also gets data from the implanted device (like a log of events, battery capacity, drug remaining, etc.) which can be displayed on the wearable or transferred to another computer for processing. Various examples are provided for wireless power transfer to implant devices.

In an embodiment, the device is configured to utilize galvanic human body communication for wireless communication, and powering, between the implanted device and a wearable device. A system includes a wearable device and an implantable device. This type of system is disclosed in WO2023163840A1. The wearable device includes one or more signal transmitters and is configured to be positioned adjacent to or in contact with a human or animal body. The implantable device includes a signal receiver and is configured to be implanted onto or within the human or animal body. The one or more signal transmitters of the wearable device are configured to transmit the signals through the human or animal body to the signal receiver of the implanted device via galvanic coupling electro-quasistatic signal transmission. The system also includes a capacitive element positioned on an electrical current flow path of the galvanic coupling between at least one of the one or more signal transmitters and the signal receiver.

Referring to FIG. 25C, two electrodes 25c72 and 25c74 of a galvanic Human Body Communication (HBC) 25c70 (e.g., prongs, coils, etc.) of the transmitter are connected to the body 25c76, and the fringe fields passing through the body 25c76 are received by the receiver 25c78 (e.g., prongs, coils, etc.). More particularly, the transmitter can transmit information through the human body over a variable frequency to the receiver 25c78 when the variable resistance of the transmitter coil is tuned to the variable resistance of the receiver coil. This is advantageous in application of an implanted device as the two electrodes 25c72, 25c74 are surrounded by and thus in contact with the body (25c76). The capacitive element is configured to restrict flow of direct current (DC) power between at least one of the one or more signal transmitters and the signal receiver.

### E. Device Configured for Self-Powering

In an embodiment, the power source is one of an energy storage device, wireless power transfer (WPT), and human body energy. The energy storage device is one or more of a biodegradable primary battery, a rechargeable battery, and a supercapacitor. The wireless power transfer may be done through near field radiation, far-field radio frequency, photovoltaic mechanism or through ultrasound.

In some embodiments, the implantable drug delivery device comprises a human body energy harvester to power its electronics. The human body energy harvester utilizes one or more triboelectric nanogenerators, piezoelectric nanogenerator or biofuel cell to gather the body's internal energy and convert it into electrical energy for the implanted device's operation. U.S. Pat. No. 3,563,245 titled Biologically Implantable and Energized Power Supply, issued Feb. 16, 1971 describes a power supply for use with a pacemaker wherein the power generator utilizes fluid pressure derived from the muscular contractions of the heart; U.S. Pat. No. 3,835,864 titled Intra-Cardiac Stimulator, issued Sep. 17, 1974 describes a stimulator for intra-cardiac use that generates electricity by using magnetic induction or by a piezoelectric effect; and U.S. Pat. No. 3,835,864 titled High efficiency vibration energy harvester, issued Jan. 10, 2006 describes an energy harvester system. These publications are hereby incorporated by reference into this disclosure for all purposes.

Referring to FIG. 25D, the biofuel cell powered electronic circuit 2500 is self-powered by a biofuel (e.g., glucose or lactate), and modulated at a low voltage, to power an integrated circuit connected to the biofuel cell contingent. The BioFuel Cell (BFC)-powered electronic circuit 2500 includes one or more integrated circuits 2520, also referred to as an electronic circuit 2520 coupled to a biofuel cell device 2510. The biofuel cell device 2510 includes an anode 2502 including a conductive electrode that is coupled to a first substrate 2506, where an enzymatic layer is formed on the conductive electrode of the anode 2502 and configured to electrochemically interact with the biofuel (e.g., glucose or lactate) in a fluid. The anode 2502 is electrically coupled to a power supply voltage terminal (e.g., VDD) of the electronic circuit 2520. The biofuel cell device 2510 includes a cathode 2504 including a conductive electrode, e.g., which can include a nanocomposite, that is coupled to a second substrate 2508. The cathode 2504 is electrically coupled to a ground voltage terminal (e.g., GND) of the electronic circuit 2520. In some embodiments, the first substrate 2506 and the second substrate 2508 are a single substrate, where the anode 2502 and the cathode 2504 are spaced apart on the single substrate. In an example, the VDD of the electronic circuit 2520 is set to a voltage of 0.25 V to 0.6 V (e.g., 0.3 V to 0.4 V in some implementations), which is near the open circuit voltage of the BFC device 2510. The electronic circuit 2520 includes a data converter 2514 to translate the power generated by the biofuel cell device 2510 to transmittable data. For example, in some embodiments, the data converter 2514 includes a delta-sigma modulation analog-to-digital (DSM ADC) converter or a ring oscillator, which is able to operate directly from the power generated by the enzymatic layer to use a maximum power point of the enzymatic BFC device 2510 to infer the biofuel concentration in the fluid. In this manner, for example, the electronic circuit 2520 is operable to translate the electrical energy as transmittable digital data that is indicative of a concentration of the analyte (e.g., biofuel) in the fluid. To control the operation of the data converter 2514, for example, the electronic circuit 2520 includes a switched or matched load 2512 coupled between the anode 2502 and the data converter 2514 to control the supply of the electrical energy (e.g., electrical current) extracted from the biofuel to the electronic circuit 2520. For example, the switched or matched load 2512 can connect the anode 2502 of the biofuel cell device 2510 to the data converter 2514, in some implementations, to establish a supply voltage at a maximum power point in a range, e.g., of 0.25 V to 0.6 V (in some examples, to the maximum power point that can include a range of 0.3 V to 0.4 V). Also, for example, in some implementations where embodiments of the data converter 2514 include the ring oscillator, the switched or matched load 2512 can connect the anode 2502 to the ring oscillator to establish a supply voltage that is set by a fixed load.

In implementations of the BFC-powered electronic circuit 2500, for example, the electronic circuit 2520 is configured to use power generated while the biofuel (e.g., glucose or lactate) is being decomposed by the enzymatic layer of the anode 2502 while also determining information about the biofuel (e.g., concentration of the biofuel in the fluid), thereby functioning as a self-powered biosensing system and bioelectronic system that can be employed in a variety of bio-related applications. In some embodiments, the electronic circuit 2520 can include a wireless transmitter 2516 in electrical communication with the data converter 2514, which can transmit the converted digital signals as data.

In some example embodiments, the electronic circuit 2520 can include analog signal conditioning circuitry, an analog-to-digital converter, or a wireless transmitter, or a combination of any two or more of the analog signal conditioning circuitries, the analog-to-digital converter, and the wireless transmitter.

### F. System Comprising Drug Delivery Device and Biosensors

An embodiment relates a system comprising an implantable device comprising: a casing that is substantially tubular and has at least a first end and a second end, opposite to the first end, a semi-permeable membrane plug at or near the first end, a first chamber, wherein one wall of the first chamber comprises the semi-permeable plug, a second chamber comprising a drug, a piston separating the first chamber and the second chamber, a third chamber comprising a flow switch and an opening for release of the drug from the implantable device into a body of a human or an animal, a fourth chamber near the second end comprising electronics, and an implantable biosensor.

In an embodiment, the biosensor is an implantable sensor. In some embodiments, the bio-chemical measurement sensor is attached directly to the implantable device to measure a concentration of the drug within the body of the human or the animal. In some embodiments, the bio-chemical measurement sensor is attached directly to the implantable device to measure a side effect biomarker of the drug within the body of the human or the animal.

In an embodiment, the biosensor is a wearable sensor. In some embodiments, the wearable sensor is configured to measure a concentration of the drug within the body of the human or the animal. In some embodiments, the wearable sensor is configured to measure a side effect biomarker of the drug within the body of the human or the animal.

In another embodiment, the biosensor is operable to detect biomakers in the body for diagnosis of diseases or side reactions that could be caused by delivery of the drug from the implantable device.

In an embodiment, the implantable device herein utilizes a closed-loop feedback control system to ensure accurate drug delivery. The implantable device comprises a closed loop unit that comprises the one or more sensors (and information/output sensed by them) and delivery of the one or more drugs and is guided by the artificial intelligence system for feedback control as described in US20140088554A1 which is incorporated with this specification in its entirety. This allows output from the one or more sensors to be used to directly modulate or control the rate of drug delivery from the drug delivery orifice, including stopping drug delivery altogether. The system is adapted to perform one or more of the following: collect the data from the one or more sensors, combine it with other diagnostic data that may have been gathered by a caregiver, perform a deep analysis of the disease state of the patient, arrive at a diagnosis, and decide whether to alter the drug delivery rate based on this diagnosis. The results of the computerized analysis of the disease state and the subsequent diagnosis can be reviewed by the caregiver and altered, modified, or rejected altogether. In these feedback loops, the caregiver can affect and control the operation of the system either through direct intervention at the point when the trigger to alter the state of the device is about to be reset, or at the point where the analysis of the disease state has been made by one or more computer executable methods that preferably utilize artificial intelligence to complete the analysis.

Referring to FIG. 26A, it shows an implantable device configured for personalized drug dose delivery, according to one or more embodiments. The implantable drug delivery device 2600 comprises one or more primary sensors 2620 and a software implemented module 2610. The one or more primary sensors 2620 measures at least one of a drug concentration in the plasma, a bioactivity marker, and a drug side effect marker. The software implemented module 2610 is configured to analyze the data obtained via the one or more primary sensors 2620 and processes the data using prediction and recommendation algorithms for determining personalized drug doses.

Referring to FIG. 26B shows a system comprising an implantable device, primary sensors and/or secondary sensors, and a processor, according to one or more embodiments. The system comprises an implantable drug delivery device 2600 comprising one or more primary sensors 2620, one or more secondary sensors 2650, 2650a, 265pb, and a software-implemented module 2670. The software-implemented module 2660 operates remotely in the cloud. The software-implemented module 2670 is configured to analyze data from the implantable device 2600, one or more primary sensors 2620, and one or more secondary sensors 2650, 2650a, 2652b, (optionally clinically relevant databases and the subject's electronic medical record (EMR)) to process data using prediction and recommendation algorithms for determining personalized drug doses.

The table below provides a list of one or more primary sensors and one or more secondary sensors that can be used with the implantable device/system:

| | | Relevant | Drug | Relevant | |
|---|---|---|---|---|---|
| | Markers of | sensor for | corresponding | sensor for | |
| Diseases | the Diseases | biomarker | to the disease | the drug | Resource |
| Diabetes | Glucose and | Continuous | Metformin; | Electrochemical | US20080161666A1 |
| | Gluconic acid | glucose | Sulfonylureas; | biosensor; | U.S. Pat. No. 11,690,577B2 |
| | concentration | monitoring | Thiazolidinediones; | (CN114994153A) | U.S. Pat. No. 7,766,830B2 |
| | Blood Glucose | sensor; | Dipeptidyl | Diffraction | KR100602952B1 |
| | Levels | Flash glucose | Peptidase-4 | based sensor | KR102315843B1 |
| | HbA1c | monitoring | Sodium-glucose | CN114609123A | US2012197148A1 |
| | Ketones | sensor; | co-transporter2; | Analyte sensor | KR20230014498A |
| | Insulin Levels | | GLP-1 Receptor | WO2007136390A1 | U.S. Pat. No. 8,615,282B2 |
| | Heart Rate | | Agonists; Insulin | | US20120059232A1 |
| | Variability | | | | doi: 10.1109/ |
| | (HRV) | | | | TBCAS.2009.201684. |
| | Skin | | | | doi: 10.1109/ |
| | Temperature | | | | TBCAS.2009.201684. |
| | | | | | doi.org/10.3390/ |
| | | | | | photonics6020071 |
| | | | | | doi: 10.1016/ |
| | | | | | j.bios.2016.03.024. |
| | | | | | doi: 10.1109/ |
| | | | | | TBCAS.2009.201684. |
| Renal | Glomerular | Wearable | Angiotensin- | Optical sensor; | CA2425337C |
| diseases | filtration | devices, | Converting | | |
| | rate; Urine | such as | Enzyme (ACE) | | |
| | Protein; | smartwatches, | Inhibitors; | | |
| | Creatinine; | wristbands, | Angiotensin | | |
| | Blood Urea | or patches, | II Receptor | | |
| | Nitrogen; | capable of | Blockers (ARBs); | | |
| | Electrolyte | analyzing | Diuretics; | | |
| | Imbalances; | sweat or | Immunosuppressive | | |
| | Renal Blood | interstitial | Drugs; | | |
| | Flow | fluid | Antihypertensive | | |
| | | | drugs; | | |
| | | | Erythropoiesis- | | |
| | | | Stimulating | | |
| | | | Agents (ESAs); | | |
| | | | Phosphate binders | | |
| Hypertension | Uric acid | — | — | Dual-mode | https://doi.org/10.1016/ |
| | | | | colorimetric | j.microc.2020.105865 |
| | | | | and | |
| | | | | fluorometric | |
| | | | | nano sensor | |
| Cardiovascular | Blood | — | warfarin | Electrochemical | https://doi.org/10.3390/ |
| diseases | prothrombin | | | sensors based | chemosensors10020044 |
| (blood vessel | time; | | | on ion selective | |
| disease, atrial | International | | | electrode | |
| fibrillation, | Normalized | | | technology | |
| stroke, | Ratio (INR) | | | | |
| pulmonary | | | | optical sensors, | US20170281771A1 |
| embolism, | | | | ultrasonic | |
| pulmonary | | | | sensors, | |
| hypertension, | | | | pressure sensors | |
| and deep vein | | | | | |
| thrombosis | | | | | |
| venous and | | | | | |
| arterial | | | | | |
| thromboembolic | | | | | |
| disorder) | | | | | |
| Cancer | proteins, mRNA, | | | Multiplexed | US20170281771B2 |
| | cytokines, | | | surface enhanced | doi: 10.1039/c5an01861g |
| | | | | Raman sensors | doi: 10.3390/s21041125. |
| HIV | HIV RNA | — | Long acting | Co-encapsulated | doi: 10.1016/ |
| | viral load | | antiretrovirals | antiretrovirals | j.ebiom.2022.104330. |
| | | | | (ARVs) with | |
| | | | | ingestible | |
| | | | | sensor (IS) | |
| Depression | elevated | | neurotransmitter, | Aptamer-based | US20190231240A1 |
| | C-reactive | | hormone, | sensor, | doi.org/10.3390/ |
| | protein in | | pharmaceutical | disposable | ijms24108480 |
| | blood | | drug, or toxin, | voltammetric | |
| | | | Trazodone (TZD), | MIP based | |
| | | | long-acting | sensor | |
| | | | antisychotics | | |
| Ocular | Intraocular | | | Implantable | EP3659495B1 |
| disease | pressure, | | | intraocular | US20120316461A1 |
| | IL-12p70; | | | physiological | https://doi.org/10.1021/ |
| | Acetyl | | | measurement | acssensors.1c00370 |
| | carnitine | | | device, contact | DOI: 10.1039/ |
| | (C2); IFN-γ; | | | lens sensor | D1AN01244D |
| | GM-CSF; IL-5; | | | | |
| | carbonic | | | | |
| | anhydrase, | | | | |
| | lipase, and | | | | |
| | antioxidants; | | | | |
| | BDNF, TFN-α; | | | | |
| | alpha-1 | | | | |
| | antichymotrypsin, | | | | |
| | Cystatin SA; | | | | |
| | protein S100-A4; | | | | |
| | Keratin (type II) | | | | |
| | etc., Lysozyme C; | | | | |
| | lacritin, | | | | |
| | lipophilin A, | | | | |
| | Ig lambda chain; | | | | |
| Acute | Abnormal | | | Tubular pressure | US20120316461A1 |
| abdominal | intra-abdominal | | | sensing device | |
| syndrome | pressure (IAP) | | | | |
| Neurodegenerative | Acetylcholine (Ach), | | Dopamine, | Carbon | https://doi.org/10.1016/ |
| Diseases | dopamine (DA) and | | acetylcholine, | Nanotube-Based | j.biochi.2017.12.015 |
| | norepinephrine (NE) | | levodopa | Fluorescent | https://doi.org/10.3390/ |
| | | | carbidopa | Nanosensors, | bios 13050499 |
| | | | | Optode-Based | doi: 10.1146/annurev- |
| | | | | Nanosensors | anchem-061417-125747 |
| Male | Hormone Levels; | Wearable | Phosphodie | | |
| reproductive | Prostate-Specific | biosensors; | sterase type | | |
| organs related | Antigen | Saliva-Based | 5 (PDE5) | | |
| ailments | | sensors; | inhibitors; | | |
| | | Breath | Alprostadil; | | |
| | | analysis | Alpha-1 blockers; | | |
| | | sensors; | 5-alpha-reductase | | |
| | | | inhibitors; | | |
| | | | leuprolide (Lupron) | | |
| | | | and bicalutamide | | |
| | | | (Casodex); | | |
| | | | docetaxel | | |
| | | | (Taxotere) or | | |
| | | | cabazitaxel | | |
| | | | (Jevtana); | | |
| | | | Clomiphene | | |
| | | | citrate; | | |
| Autoimmune | Antigliadin Abs (AGA) | | | immunosensors | doi: |
| diseases | and anti-transglutamin | | | | 10.3390/bios9010038. |
| [Celiac | ase Abs (anti-(TG) | | | | |
| disease | | | | | |
| (CD)] | | | | | |
| Asthma | FeNO(inhaled | | Corticosteroids | Sensor based | doi: |
| | corticosteroid (ICS) | | and short-acting | inhaler | 10.1542/peds.2020-1330. |
| | adherence) | | β-agonist | | |
| | | | medications | | |
| Infectious | tracheal cytotoxin | | | multiplexed | U.S. Pat. No. 10,603,650B2 |
| disease | (TCT), Hemozoin | | | surface | U.S. Pat. No. 8,774,884B2 |
| | nanoparticles | | | enhanced | https://doi.org/ |
| | | | | Raman sensors, | 10.3390/s20143953 |
| | | | | Electromagnetic | https://doi.org/ |
| | | | | energy sensor, | 10.3390/nano11040840 |
| | | | | Aptamer based | |
| | | | | sensor | |

### G. System for Precision Dosing of a Drug

The AI system analyzes drug delivery device usage and drug delivery data, learning from the patterns to predict clinical outcomes. By understanding these patterns, the AI system can adjust drug dosages to optimize patient outcomes. For example, the AI system processes a diabetic patient's insulin injector usage data and predicts their blood glucose level response based on their adherence to a prescribed insulin delivery schedule.

The AI processes drug delivery device usage and drug delivery data aggregated with clinical outcome data, such as self-reported symptoms and sensed data, to determine the therapeutic benefit of a drug and the occurrence of side effects. By analyzing correlations between specific drugs or drug delivery schedules and certain side effects, the AI can tailor treatment plans to individual patients. This helps to develop new drugs or drug delivery regimens that produce desired clinical outcomes for a patient population.

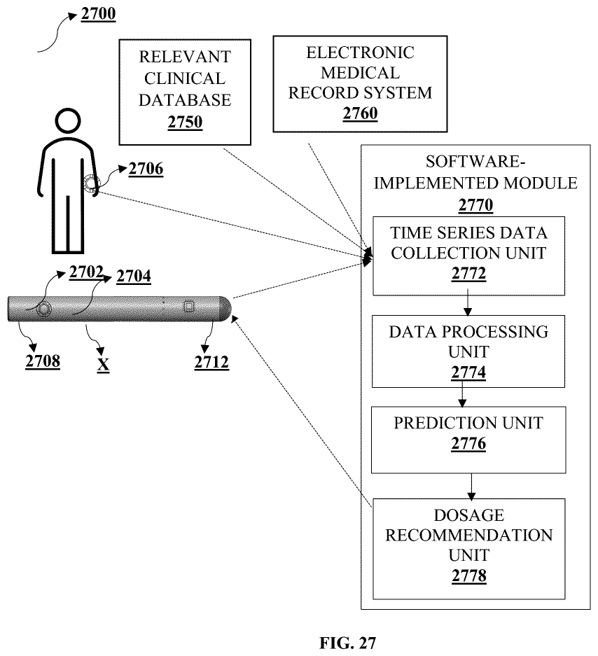

Referring to FIG. 27, it shows a system for precision dosing of a drug, according to one or more embodiments. The system 2700 comprises an implantable drug delivery device 2702 comprising one or more primary sensors 2704, one or more secondary sensors 2706, and a software-implemented module 2770.

The implantable drug delivery device 2702 comprises a reservoir 2708 comprising a body-temperature-stable drug X, microfluidic channels 2710 for controlled drug delivery, microelectronic circuitry 2712 for sensor communication and power management, and one or more primary sensors 2704 to monitor the implantable device's function, as well as physiological parameters such as heart rate, blood pressure, and respiration. The one or more secondary sensors 2708 are configured to measure at least one of the drug concentration and biomarkers in the subject's body. The one or more secondary sensors 2706 further comprise activity trackers or environmental sensors for additional context. The software-implemented module 2770 operates on the drug delivery device 2702 or remotely in the cloud. The software-implemented module 2770 is configured to analyze data from the implantable device 2702, sensor readings, clinical databases 2750, and the subject's electronic medical record (EMR) 2760 to process data using prediction and recommendation algorithms for determining personalized drug doses.

The software-implemented module 2770 comprises a time series data collection unit 2772, a data processing unit 2774, a prediction unit 2776, and a dosage recommendation unit 2778. The time series data collection unit 2772 collects data from the implantable drug delivery device 2702, the one or more primary sensors 2704, one or more secondary sensors 2706, the microelectronic circuitry 2712, clinical databases 2750, and the subject's electronic medical record (EMR) 2760. The data processing unit 2774 preprocesses sensor data and EMR information for analysis, identifying patterns and trends in physiological and clinical data while filtering out noise and irrelevant information. The prediction unit 2776 comprises prediction algorithms and machine learning models trained on large datasets of patient data and is configured to predict the potential effectiveness and side effects of different drug doses for a specific patient and target disease. The machine learning model further analyzes individual variations in metabolism, pharmacodynamics, and disease progression. The recommendation unit, comprising a recommendation algorithm, is configured to determine an effective and tolerable drug dose based on predictions. The algorithm assesses the target disease status, current physiological state, and medication history, dynamically adjusting the dose in response to real-time changes in the subject's condition. The machine learning model comprises a drug prediction model, a complex algorithm trained on extensive data, including patient demographics, medical history, disease-specific biomarkers, genetic information, drug efficacy, side effect profiles, pharmacokinetics, pharmacodynamics of the drug, and environmental and lifestyle factors. The model learns to predict the optimal dose for each individual, balancing therapeutic effectiveness with minimal side effects.

The system is designed to a) provide tailored drug delivery based on individual needs, improving efficacy, and reducing side effects, b) enhance adherence to treatment plans through continuous monitoring and automatic dose adjustments, c) enable early detection of potential complications and proactive interventions through real-time data analysis, and d) lead to cost savings through optimized drug use and improved treatment outcomes.

### H. Method of Manufacturing the Device

An embodiment relates to a method of manufacturing the described device. The manufacturing process starts by acquiring a tube with a built-in stopper. The piston is carefully placed within the tube to rest on the stopper, preventing any further downward movement. The initial configuration is set with a total liquid chamber volume of approximately between 200 μl to 1000 μl, more preferably to 300 μl to 800 μl, and most preferably to 400 μl to 600 μl combining the first and second chambers. A total liquid chamber volume 500 μl is used as one example and is not limiting. A stopper mechanism is integrated to impede the piston's movement toward the fourth chamber. The assembly of the first chamber comprises positioning the tube vertically, allowing the piston to rest on the stopper, and filling the drug chamber to a specified level via the drug delivery orifice. The tube is configured with threading or soldering on one side, incorporating a stopper on the side of the first chamber and a built-in plate on the side of the fourth chamber with a small drug orifice. The tube is then placed horizontally or vertically, enabling the piston to rest on the stops, and the drug chamber is filled with 400 microliters of the drug suspension through the drug delivery orifice. Five micrograms of sodium chloride are added to the salt chamber to generate pressure, and the tube is capped to create a sealed environment.

In some embodiments, the process of tube fabrication comprises starting with the fabrication of a metal tube using common materials such as titanium, stainless steel, or bioinert alloys. Techniques utilized include precision machining (CNC), laser cutting, or electrochemical machining to achieve the desired dimensions. It is crucial to ensure that the tube dimensions match the required volumes for drug and saline. Next, the production of back and front-end caps is carried out separately. For plastics, CNC machining or injection molding is used, while metal end caps are produced through precision turning. The surface finish of these end caps must be smooth and biocompatible. Moving on to the drug chamber assembly, the piston is inserted into the metal tube, and the front-end cap is attached, ensuring proper alignment. The front chamber is then filled with the drug suspension, and the front-end cap is securely sealed. Similarly, for the saline chamber assembly, the first end cap is attached to the tube, the back chamber is filled with osmotic agent, and the first end cap is sealed securely. The overall assembly process comprises joining the front and back assemblies by sliding them onto the metal tube, ensuring proper alignment and secure attachment. This assembly is tested for leaks and functionality before proceeding to sterilization. Finally, the assembled device undergoes sterilization using methods such as gamma radiation or ethylene oxide. It is then packaged for surgical use as an implantable device.

To transmit electrical signals from and to the second chamber, third chamber and/or first chamber to the fourth chamber, where the electronics are located, a method comprises coating the external side of an FDA approved metallic tube (e.g., titanium tube) with a non-conductive ceramic or polymer or epoxy resins. The wires running on or through this non-conductive layer could be vapor-deposited metallic wires, as thin as a few nanometers, forming PCB tracks. These tracks can carry signals from the first, second, and third chambers to the fourth chamber. To isolate the PCB tracks from the external environment, a polymer layer is applied. The device is sealed with an end cap on the second end of the device, and functionality is verified, ensuring that the conductance and pressure sensors transmit signals reliably.

In one embodiment, the device comprises circuits on the top and inside of the tube made using the MicroPen technology. Micro Pen technology refers to a precision writing or printing technique at the microscale, typically involving the deposition of materials with extremely fine resolution. This technology is employed for various applications, including drawing electric circuits on small surfaces. A specialized Micro Pen device is utilized, equipped with a fine-tip pen or nozzle capable of depositing conductive ink or material with high precision. The micro pen dispenses conductive ink or material that can create electrically conductive pathways. This ink is designed to adhere to the inner surface of the tube. The inner surface of the tube diameter below 10 mm should be appropriately prepared to ensure adhesion and compatibility with the conductive ink. Surface treatment or coating is necessary to optimize the substrate for circuit drawing. Micro Pen technology offers exceptional resolution and precision, allowing for the creation of microscale patterns and circuits, crucial when working within the confined space of a tube. After the circuit is drawn, there is a curing or drying process to solidify the conductive material and enhance its stability on the inner surface of the tube. The electric circuits drawn using Micro Pen technology can be integrated with microelectronics, sensors, or other components within the tube, enabling the creation of miniaturized electronic devices. The choice of conductive ink or material is crucial to ensure compatibility with the tube substrate, adherence properties, and electrical conductivity.

In some embodiments, the device further comprises a cylindrical flexible circuit that has a dielectric layer in a cylindrical form, a monomer layer covalently bonded to the dielectric layer, a conductive layer adhered to the monomer layer, a device element, and an open, unfilled lumen. The dielectric layer comprises a thermoplastic, such as Pebax. In one embodiment the dielectric layer and the shaft body are substantially the same material.

A flexible circuit utilizes a flexible substrate, typically made with a thin flexible plastic or metal foil as the substrate. The flexible circuit is advantageously long, thin, and narrow. Often, the flexible substrate utilizes an insulating material or a dielectric. The substrate can be made for example from thermoset or thermoplastic polymers. The substrate can be made from polymers such as liquid-crystal polymers (LCP), polyether ether ketone (PEEK), polyester, Polyethylene terephthalate (PET), polyimide (PI) (e.g., DuPont Pyralux®), polyethylene napthalate (PEN), polyetherimide (PEI), polyefine, Kapton, various fluropolymers (FEP), PTFE, silicone, parylene, reinforced composites and copolymer Polyimide films or a transparent conductive polyester film or other dielectrics. One non-limiting example of the latter is the product Topas® COC (TOPAS Advanced Polymers GmbH, Oberhausen, Germany or Ticona GmbH, Kelsterbach, Germany). In some cases, the flexible substrate comprises a first polymer coated by a second polymer that provides superior adhesion to the next layer, for example, parylene is a second polymer coating. Advantageously the material for the flexible circuit is biologically inert or biocompatible. In some embodiments, the flexible circuit is covered with a layer of biocompatible hydrogel, silicone, PTFE, for example, to reduce the friction and for improved biocompatibility, e.g., to avoid blood coagulation.

In one embodiment the conductive layer comprises a seed layer and a trace layer. The seed layer comprises a metal or metal ion selected from the group consisting of palladium, ruthenium, rhodium, osmium, iridium, platinum, silver, copper, and their ions, or a conductive polymer. When the seed layer is a conductive polymer, the seed layer and the monomer layer can comprise the same monomer, or different monomers copolymerized.

In one embodiment the trace layer comprises a metal or metal ion selected from the group consisting of copper, silver, gold, nickel, titanium, and chromium. The seed layer and the trace layer comprise a combination of the same or different metal or ions thereof.

In another embodiment the device's monomer layer is covalently bonded to the dielectric layer in a desired conductive pattern. The monomer layer has a carboxylic group.

In one embodiment, the device comprises a conductive trace that is between 5 μm and 20 μm wide. The flexible circuit can be between 1 μm and 30 μm thick and can exclude any adhesive layer between the dielectric layer and the conductive trace layer.

In another embodiment, the device comprises an elongated lumen shaft that further comprises a cylindrical flexible circuit that has a dielectric layer in a cylindrical form, a monomer layer covalently bonded to the dielectric layer, a conductive trace layer adhered to the monomer layer, a medical device element, and an open, unfilled lumen.

In some embodiments, a flexible circuit comprises a base layer such as a polyimide film. The base layer is as thick as required for the device, but is preferably relatively thin, in the range of 5 μm to 125 μm, preferably 10-20 μm. Several different materials are suitable for base layers, such as polyester, Polyethylene terephthalate (PET), polyethylene napthalate (PEN), Polyetherimide (PEI), along with various fluoropolymers such as (FEP) and copolymers. While polyester films are the most cost effective, polyimide films are often preferred in a medical device setting due to their blend of electrical, mechanical, chemical, and thermal properties. In some embodiments, the flexible circuit has an adhesive layer, which is typically selected for its ability to bond the base layer to the electrical layer, and for its thermal properties (e.g., ability to retain a bond in the operational temperature range). Many flex circuits use adhesive systems from the different polymer families, including polyimide adhesives, polyester adhesives, acrylics, epoxies, and phenolics. The adhesive is a thermoset or a thermoplastic adhesive. As with the base films, adhesives come in different thicknesses. Thickness selection is typically a function of the application.

In some embodiments, an FDA approved polymer, e.g., epoxy, can be coated on the inside and outside of the metal, e.g., titanium or stainless steel, tube to form insulating layers on the inner and outer surfaces of the metal tube.

In an embodiment, the device comprises thin walled elongated hollow lumen structures comprising at least in part of a cylindrical flexible circuit to create thin walled structures, having simplified manufacturability along with complex functionality. The cylindrical flexible circuit is configured in such a way to carry at least part of the device's structural loads and therefore reduce the medical device total wall thickness. The device has a structure that includes a flexible circuit comprising one or more dielectric layers (such as polyimide, silicone, parylene, LCP, ceramic, reinforced composites, for example), and one or more electrically conductive layers (such as copper, silver, carbon, conductive inks, for example), and possibly one or more mounted electronic components (such as electrodes, thermistors, capacitive micromachined ultrasonic transducers, pressure sensors, for example), which may be mounted in, on or within the elongated open lumen body over part or the whole of its length. Flexible circuits are known in the industry under a variety of names, including flexible printed wire boards (PWB), flexible electronics, flexible printed wiring, flexible printed circuit board (PCB), flexible printed wire assembly (PWA), flexible printed circuit assembly (PCA), or flexible printed circuit board assembly (PCBA).

In an exemplary embodiment of the invention, a flexible circuit overall, or part of, its length is rolled into a tubular, or partial tubular, shape such that the seam or edges run longitudinally down the length of the shaft, covering all or part of the circumference of an open lumen tubular shaft material such as a stainless hypo tube, a polymer catheter shaft, for example. The seam or edges are affixed together by means of an adhesive or a thermoplastic reflow process, for example. Alternatively, a thermoset dielectric may be induced to hold a tubular or partial tubular shape through use of a stress relieving process. The latter may serve to hold the edges of the flexible circuit together over its length, but both the flexible circuit or the open lumen tubular shaft material may comprise the load bearing structure of the invention.

Medical devices with integrated flexible circuit and methods of making the same are described in US20220225940A1, US20190254607A1, and WO2023224521A1, and incorporated herein by reference.

An embodiment relates to an AI-based implantable drug delivery system that works like an autopilot, which continuously a drug level or a biomarker and delivers a drug to keep a disease under control. Utilizing state-of-the-art technologies and interdisciplinary methods can enhance drug delivery, improve patient outcomes, and push medical science boundaries. The AI-based implantable drug delivery system is a major shift in drug delivery, akin to an autopilot within the body, operating with precision to administer drugs based on individual needs. This autopilot-type drug delivery technology comprises four components:

Implantable Mini Pump: Serves as the delivery engine, dispensing drugs subcutaneously in a controlled and on-demand manner.

Body Temperature Stable Drug Formulation: Ensures that the drug does not degrade in the human body at body temperature for at least 6 months (preferably, 1 year, more preferably 2 years, and most preferably 3 years).

Implantable Biosensor: Provides real-time monitoring of a drug level and/or a health-related biomarker, offering valuable insights for personalized treatment for triggering by either the skin touch or the integrated sensor device.

AI Integration: Integrates and analyzes data to optimize drug delivery in real-time, ensuring patient-specific care.

In an embodiment, the implantable device will be an on-demand tubular implantable device of about 3-6 mm diameter and two 3-6 cm long tubes connected via a flexible joint. The first tube contains a pressurized chamber, a piston separating the pressurized chamber and a drug chamber containing a drug such as naloxone, and an on-off flow switch, which is a combination of a relief valve and an electromagnetic actuator such as a stepper motor, to initiate and control the flow of the drug from the implantable device into the human body. The second tube contains the electronics and power supply. The amount of the drug delivered from the first tube can be adjusted for different body weight.

Integrating these pillars, the device not only improves drug delivery efficiency and precision but also shifts towards patient-centered, data-driven treatments

### AA. Use of Artificial Intelligence in the Device

In some embodiments, the implantable drug delivery device can be equipped with sensors to monitor various physiological parameters such as heart rate, blood pressure, and glucose levels. Artificial intelligence algorithms can analyze this data in real-time to optimize drug dosages and timing based on the patient's needs. In some embodiments, the AI algorithms can analyze a patient's medical history, genetic makeup, and other factors to personalize drug dosages for each patient. This can improve treatment efficacy and reduce the risk of adverse side effects. In some embodiments, the AI algorithms can analyze large amounts of patient data to predict which patients are most likely to experience adverse drug reactions or drug interactions. This can help healthcare providers proactively adjust treatment plans to avoid complications. In some embodiments, the AI can enable remote monitoring and control of implantable drug delivery devices. Healthcare providers can monitor patient data in real-time and adjust drug dosages and timing as needed, even from a remote location.

Embodiments are provided for a method for generating a software-implemented module configured to determine a drug dose, for training a machine learning model to generate a software-implemented module and for determining a drug dose.

### Artificial Intelligence Module Comprising Feedback Loop

In some embodiments, the artificial intelligence module can continuously learn and adapt based on feedback about its performance. The feedback loop is a critical component of the AI unit, allowing it to improve its accuracy and effectiveness over time. The feedback loop can be either positive or negative. A positive feedback loop occurs when the system receives feedback that reinforces its behavior, leading to more of the same behavior. A negative feedback loop occurs when the system receives feedback that signals it to change its behavior.

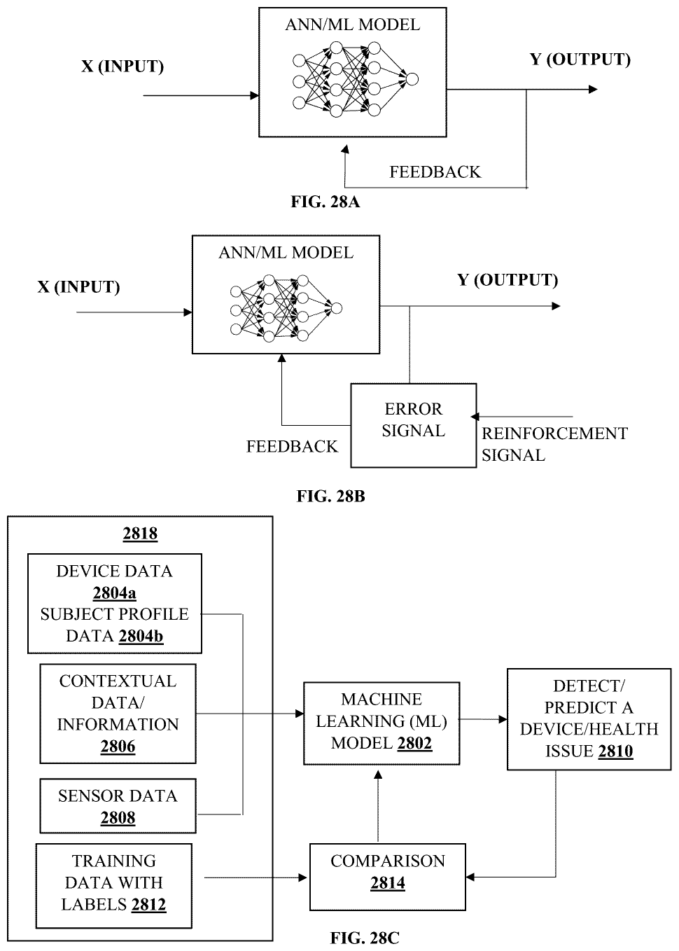

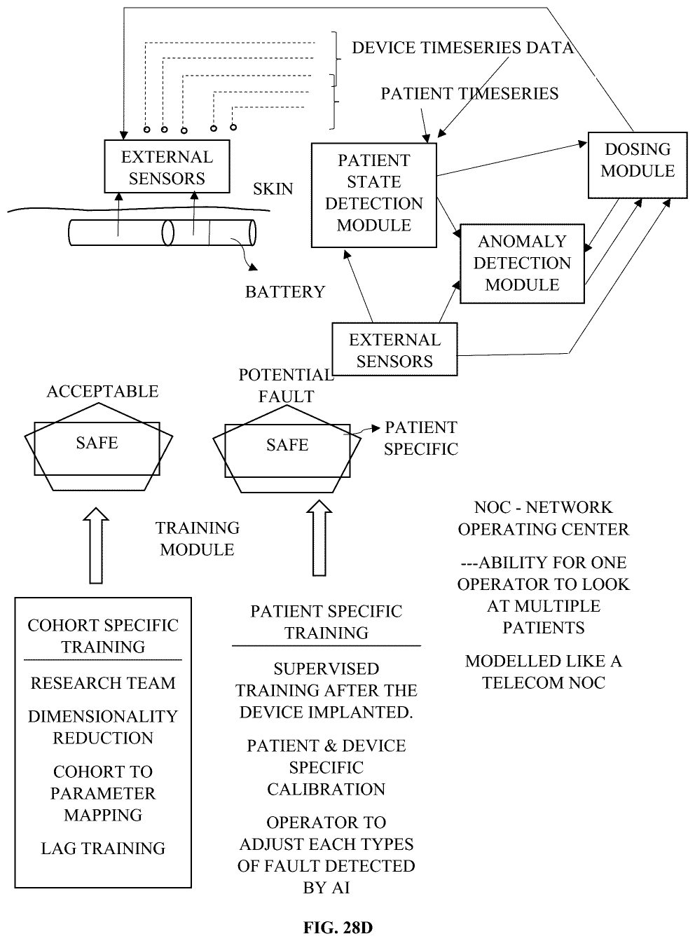

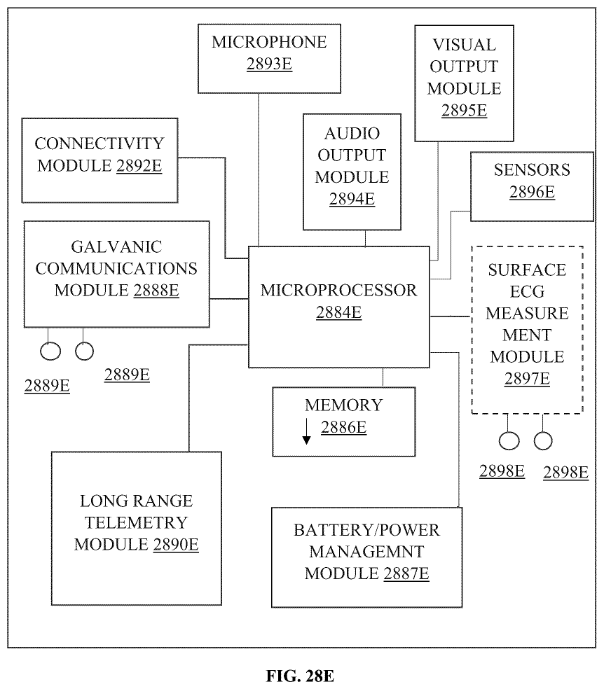

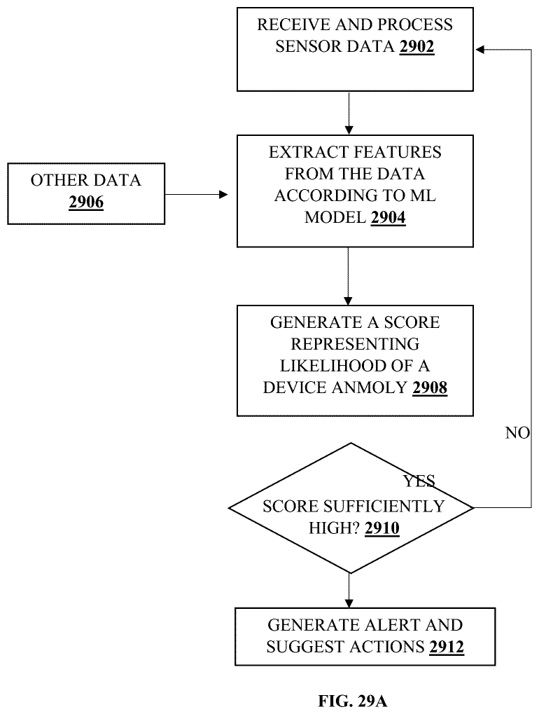

FIG. 28A shows a structure of a neural network/machine learning model with a feedback loop. Artificial neural networks (ANNs) model comprises an input layer, one or more hidden layers, and an output layer. Each node, or artificial neuron, connects to another and has an associated weight and threshold. If the output of any individual node is above the specified threshold value, that node is activated, sending data to the next layer of the network. Otherwise, no data is passed to the next layer of the network. A machine learning model or an ANN model is trained on a set of data to take a request in the form of input data, make a prediction on that input data, and then provide a response. The model may learn from the data. Learning can be supervised learning and/or unsupervised learning and is based on different scenarios and with different datasets. Supervised learning comprises logic using at least one of a decision tree, logistic regression, and support vector machines. Unsupervised learning comprises logic using at least one of a k-means clustering, a hierarchical clustering, a hidden Markov model, and an apriori algorithm. The output layer predicts or detects a health issue and the severity of the health issue based on the input data.

In an embodiment, ANN's is a Deep-Neural Network (DNN), which is a multilayer tandem neural network comprising Artificial Neural Networks (ANN), Convolution Neural Networks (CNN) and Recurrent Neural Networks (RNN) that can recognize features from inputs, do an expert review, and perform actions that require predictions, creative thinking, and analytics. In an embodiment, ANNs is Recurrent Neural Network (RNN), which is a type of Artificial Neural Networks (ANN), which uses sequential data or time series data. Deep learning algorithms are commonly used for ordinal or temporal problems, such as language translation, Natural Language Processing (NLP), speech recognition, and image recognition, etc. Like feedforward and convolutional neural networks (CNNs), recurrent neural networks utilize training data to learn. They are distinguished by their “memory” as they take information from prior input via a feedback loop to influence the current input and output. An output from the output layer in a neural network model is fed back to the model through the feedback loop. The variations of weights in the hidden layer(s) are adjusted to fit the expected outputs better while training the model. This allows the model to provide results with far fewer mistakes.

The neural network is featured with the feedback loop to adjust the system output dynamically as it learns from the new data. In machine learning, backpropagation and feedback loops are used to train an AI model and continuously improve it upon usage. As the incoming data that the model receives increases, there are more opportunities for the model to learn from the data. The feedback loops, or backpropagation algorithms, identify inconsistencies and feed the corrected information back into the model as an input.

Even though the AI/ML model is trained well, with large sets of labeled data and concepts, after a while, the models' performance may decline while adding new, unlabeled input due to many reasons which include, but not limited to, concept drift, recall precision degradation due to drifting away from true positives, and data drift over time. A feedback loop to the model keeps the AI results accurate and ensures that the model maintains its performance and improvement, even when new unlabeled data is assimilated. A feedback loop refers to the process by which an AI model's predicted output is reused to train new versions of the model.

Initially, when the AI/ML model is trained, a few labeled samples comprising both positive and negative examples of the concepts (for e.g., health issues) are used that are meant for the model to learn. Afterward, the model is tested using unlabeled data. By using, for example, deep learning and neural networks, the model can then make predictions on whether the desired concept/s (for e.g., health issues that need to be detected) are in unlabeled images. Each dataset is given a probability score where higher scores represent a higher level of confidence in the models' predictions. Where a model gives an image a high probability score, it is auto labeled with the predicted concept. However, in the cases where the model returns a low probability score, this input is sent to a controller (maybe a human moderator) which verifies and, as necessary, corrects the result. The human moderator is used only in exceptional cases. The feedback loop feeds labeled data, auto-labeled or controller-verified, back to the model dynamically and is used as training data so that the system can improve its predictions in real-time and dynamically.

FIG. 28B shows a structure of the neural network/machine learning model with reinforcement learning. The network receives feedback from authorized networked environments. Though the system is similar to supervised learning, the feedback obtained in this case is evaluative not instructive, which means there is no teacher as in supervised learning. After receiving the feedback, the network performs adjustments of the weights to get better predictions in the future. Machine learning techniques, like deep learning, allow models to take labeled training data and learn to recognize those concepts in subsequent data and images. Data is fed back to the model for testing, hence by feeding the model with data it has already predicted over, the training gets reinforced. If the machine learning model has a feedback loop, a reward for each true positive of the output of the system is given to further reinforce the learning. Feedback loops ensure that AI results do not stagnate. By incorporating a feedback loop, the model's output keeps improving dynamically and over usage/time.

### 1. Use of Artificial Intelligence to Check Postoperative Performance of the Implanted Device

FIG. 28C shows an example block diagram for detecting one or more prognosticators, indicators, or risk factors of postoperative performance of the implanted device using a machine learning model. The machine learning model 2802 may take as input any data associated with the subject and/or the implant and learn to identify features within the data that are predictive of a personalized treatment or a device related anomaly. Predicting a personalized treatment refers to designing a drug dose and a drug delivery rate based on one or more prognosticators and indicators such as current health parameters and historical health parameters. Predicting a device anomaly refers to predicting a future event based on past and present data and most commonly by analysis of trends or data patterns of the device parameters comprising the duration of device implant, delivery rate, the pressures inside each chamber of the device, the performance of electronic components, and data from the sensors of the device, and the biochemical changes around the device in the body of a subject.

Any of the device data 2804a (e.g. the duration of device implant, delivery rate, the pressures inside each chamber of the device, the performance of electronic components, and data from the sensors of the device, etc.) that correlates with device performance, subject profile data 2804b (e.g. subject's age, weight, height, kidney, or liver function, etc.), preconditions from a first database (e.g., an EHR database containing information about the patient, vitals, ethnicity, etc.), contextual information 2806, sensor data 2808, or any other data (e.g. other currently administered medications presently in the patient's tissue, effects of the medication or pre-existing medications, for example, blood pressure, pulse, heart rhythm, or respirations, change in physical activity, travel, change in food habits, etc.) that may correlate with the subject's general health condition and disposition, and such correlation may be automatically learned by the machine learning model 2802. In an embodiment, during training, the machine learning model 2802 may process the training data sample (e.g., device data 2804a, subject profile data 2804b and/or contextual information 2806), and, based on the current parameters of the machine learning model 2802, detect or predict a device issue or a health issue 2810. The detection of a one or more prognosticators, indicators, or risk factors of postoperative performance of the implanted device and prediction of a device issue or a health issue 2810 may depend on the training data with labels 2812 associated with the training data sample 2818. Prediction or predictive analysis employs probability based on the data analyses and processing. For example, if the training data with labels 2812 indicates a biochemical or biophysical change in the surrounding of the device implanted in the subject, the machine learning model 2802 would learn to detect the issue based on input data associated with a given device data and subject profile 2804, contextual information 2806 and/or sensor data 2808. In an embodiment, during training, the device or health issue 2810 and the training data with labels 2812 may be compared at 2814. For example, the comparison 2814 may be based on a loss function that measures a difference between the detected device or health issue 2810 and the training data with labels 2812. Based on the comparison 2814 or the corresponding output of the loss function, a training algorithm may update the parameters of the machine learning model 2802, with the objective of minimizing the differences or losses between subsequent predictions or detections of the device or health issue 2810 and the corresponding training data with labels 2812. By iteratively training in this manner, the machine learning model 2802 may “learn” from the different training data samples and become better at detecting various device and/or health issues 2810 that are similar to the ones represented by the training data with labels 2812. In an embodiment, data which is specific to the drug type, the implant device, the location of implant, and the patient in which the implant is placed, for which the model is used for detecting postoperative performance can be used to train the machine learning model 2802. In an embodiment, data, which is general to the health condition of a subject and device, is used to train the machine learning model, and the updated model can be used for detecting a postoperative performance of the implanted device for any other subject.

### 2. Use of AI for Addressing to a Device Anomaly

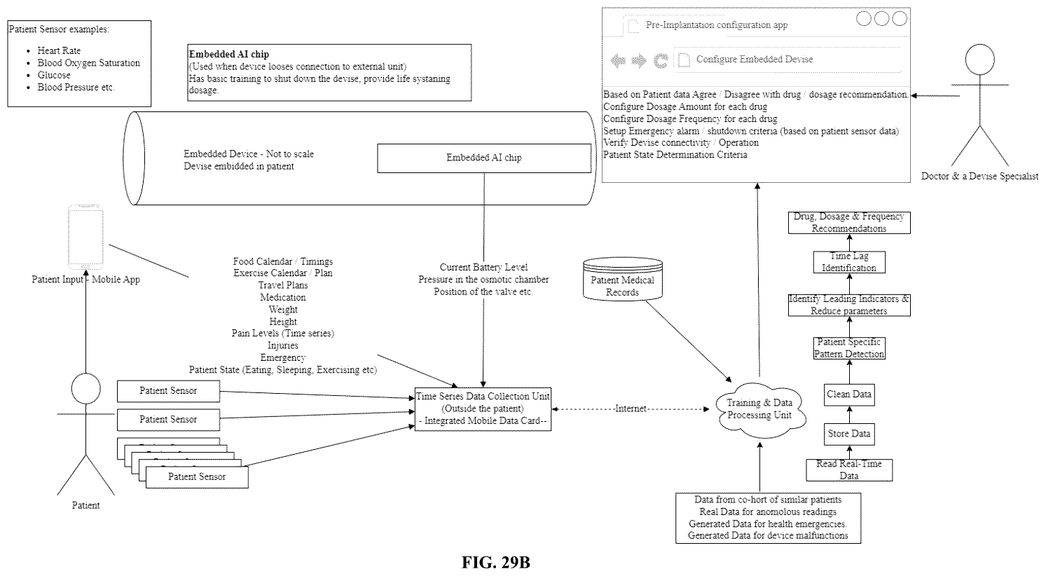

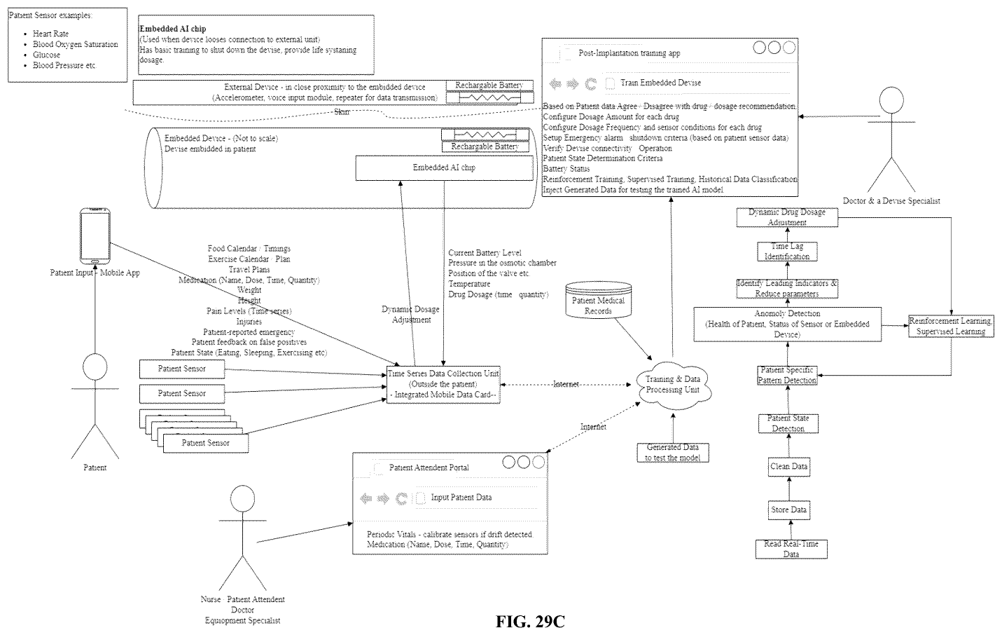

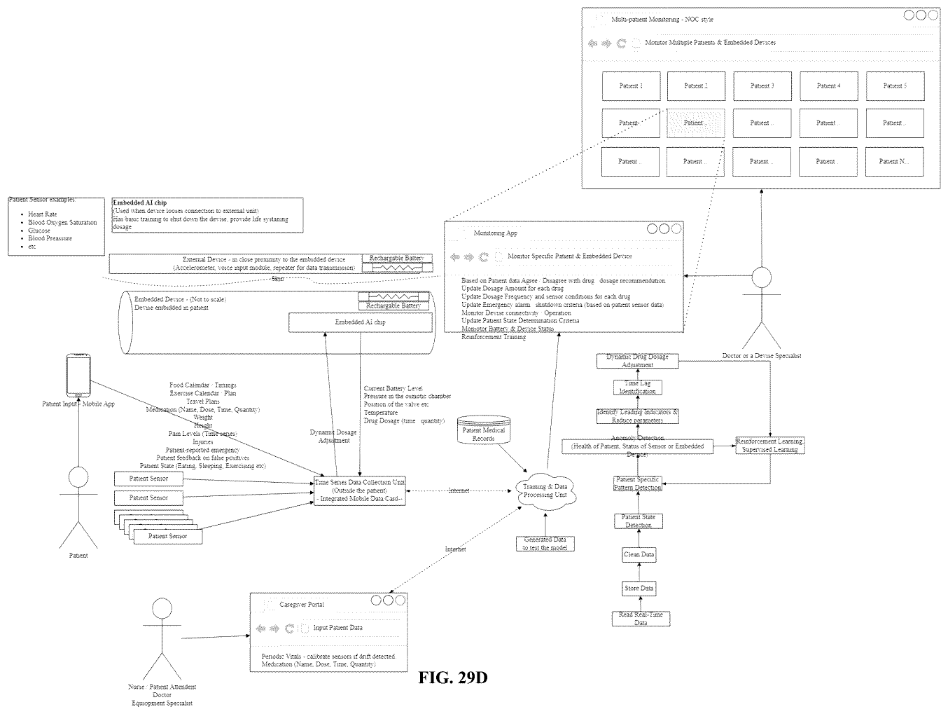

FIG. 29A shows an example flow chart for detecting a device anomaly using a machine learning model. The device anomaly may comprise an implant failure and/or side effects associated with the implantable device. The system may receive data associated with sensor output(s) from one or more sensors 2902 from the implantable device. Data pertaining to the performance of the implantable device can be gathered using any type of a sensor output, for example, pressure measurement, a leakage, infrared measures, temperature measures, or any other information measured or detected by sensors. In an embodiment, a sensor output may be the result of one or more sensors capturing environmental information associated with the subject having the device implanted. The system may receive other data 2906, for example, from wearable devices, external electronic medical record databases, and attachable monitoring devices. The machine learning model 2904 of the system can process the data to generate a score representing likelihood of a device anomaly 2908. If the score is sufficiently high 2910 the system sends an alert and suggests actions 2912 via a user device. The user device may comprise a mobile phone or a laptop.

### Artificial Intelligence Based Implantable Device:

In an embodiment, the implantable drug delivery device is an artificial intelligence based device. The integration of the artificial intelligence engine into the implantable device enables real-time monitoring of patient health data. The integration of the artificial intelligence into the implantable device further allows for adjustments in medicine dosage as needed, and identifying potential faults in the system. The integration of the artificial intelligence into the implantable device enables monitoring of the current health condition of the patient in real-time and in response to the current health condition of the patient dynamically alters the dosage in real-time.

The system comprising the implantable device is centered around an artificial intelligence (AI) and machine learning (ML) system that is designed to work with embedded drug delivery devices. The system aims to optimize drug dosage and detect anomalies in device usage and patient data. The AI and ML system is built upon predictive models and artificial neural networks that can analyze data and predict clinical outcomes. The technical details are explained below:

### Dosage Adjustment:

The AI system analyzes drug delivery device usage and drug delivery data, learning from the patterns to predict clinical outcomes. By understanding these patterns, the AI system can adjust drug dosages to optimize patient outcomes. For example, the AI system may process a diabetic patient's insulin injector usage data and predict their blood glucose level response based on their adherence to a prescribed insulin delivery schedule.

### Anomaly Detection:

The AI system also performs trend analysis on the drug delivery device usage and drug delivery data to identify trends, variations, and anomalies over time. Comparing this data to desired or predetermined patterns of usage, delivery, and clinical outcomes, the AI system determines compliance and expected clinical results. This information enables doctors to monitor and manage a patient's treatment more effectively, potentially leading to adjustments in treatment plans or even replacement with alternative treatments.

The machine learning component of the system can take various forms, including artificial neural networks, which consist of nodes (artificial neurons) organized in layers (input, hidden, and output). The learning process involves updating connection weights between nodes and using activation functions to convert weighted inputs to node outputs.

By continually learning from sensed signatures and adapting to physiological changes in individuals, the AI system can maintain its functionality even when the individual's physiological functions change over time which enables personalized feedback loops, performance enhancement, and better overall patient care.

Moreover, the AI system learns from operators, nurses, patients and doctors and further improve the dosage adjustment recommendations and anomaly detection. The AI system uses this information as input for the enciphered network, tailoring the stimulation patterns to better suit individual needs.

### Data Sources:

To effectively detect anomalies and control pharmaceutical drug dosage, the AI system reads from multiple data sources comprising systems and sub-systems of Table 1. Some of these sources are listed below:

### Device-Related Data:

Battery Voltage
Derived: Average Voltage
Derived: Voltage change during communication and pump operation
Valve State (open, close, position)
Derived: Rate of valve opening or closing
Derived: Valve stuck in open or close position
Derived: Actuator shorted/open (including intermittent issues)
Seal Leakage: valve input to output

### Processor Operations: The Failures/Anomalies in Processor Operations Comprises the Following:

Derived: Failure state and reset
Derived: Watchdog timer reset

### Communication: The Failures/Anomalies in Communication Comprises the Following:

Transmitter or Receiver Failure
Lack of communication for a certain duration

### Pharmaceutical Product Dosage Data: The Failures/Anomalies Herein Comprises the Following:

Dosage administered.

### External Device-Related Data:

Motion sensor data (to detect the patient's state)
Microphone data from the device
Patient biometrics, such as:
Oxygen sensor readings
Insulin levels
Heart rate
Blood pressure
Pupil dilation, etc.

### Patient or Care Provider Input:

Regular feedback from the patient or care provider on the patient's condition, additional medications taken, etc. This can be received through a customized questionnaire on a mobile app or from the microphone attached to the external portion of the device.

The AI system, performing Anomaly detection, involves analyzing individual time-series data sets or combinations of these data sets. Anomalies may also be identified if the predicted outcome after dosage administration does not align with the expected outcome. Implementing an appropriate algorithm to analyze these data sources will help to detect anomalies and adjust the medicine dosage accordingly.

### Data Preparation & Adjustments:

The AI engine performs data preparation, adjustment, and dimensionality reduction for anomaly detection and dosage adjustment recommendation AI.

Data preparation and adjustment are crucial steps in creating an effective AI system for anomaly detection and dosage adjustment recommendation. The data input for the AI can be enhanced by implementing time lag adjustments, parameter mapping for dimensionality reduction, and combining correlated parameters with specific weights. The following improvements can be made to the data preparation and adjustment process:

### Data Cleaning and Preprocessing:

Prior to feeding data into the AI system, statistical methods such as Autoregressive Integrated Moving Average (ARIMA), Exponential Smoothing State Space Model (ETS), and Seasonal Decomposition of Time Series (STL) can be used to preprocess and clean the data.

This step ensures that the AI system receives high-quality input data, which will result in more accurate predictions and recommendations for dosage adjustment and anomaly detection.

By implementing these improvements in data preparation and adjustment, the AI system for anomaly detection and dosage adjustment recommendation becomes more efficient and accurate, ultimately benefiting both patients and healthcare providers.

### Time Lag Calculations and Adjustments:

Identifying the time lag between various time series datasets is critical for both dosage adjustment and anomaly detection. This includes determining the delay between pharmaceutical administration and observed changes in patient biometrics using statistical techniques like cross-correlation analysis and Granger causality test.

Based on the identified time lag, the AI system can predict patient outcomes and inform doctors about the expected reactions to drug administration. A feedback loop can be established to adjust dosage amount, frequency, or timing based on patient states (e.g., asleep, post-meal, post-exercise, awake).

### Dimensionality Reduction and Parameter Mapping:

Each time-series data is a parameter. The data sets received are all time series in nature, because they all keep changing with time. The AI engine performs a mapping between these parameters and groupings of patients by grouping. Certain parameters are important for certain health conditions and for other health conditions, these parameters are not as relevant. The AI prediction models work better when there are fewer parameters. Reduced number of parameters improve model efficiency, reduces model complexity, allows easy creation of training data, and prevents overfitting due to less amount of training data. Training data needs grow exponentially with number of parameters. There are several ways the parameters can be reduced. This can be based on prior research for effect of pharmaceutical drug, reduction of dimensionality by merging parameters using techniques like Principal Component Analysis (PCA) or t-Distributed Stochastic Neighbours Embedding (t-SNE). By independently evaluating each parameter, we can identify which parameters co-relate most strongly with bio-metric data for patient health condition and patient states (like: asleep, eating, awake, walking, running etc). After the most impacted biometrics are identified, they are selected for further dimensionality reduction, by identifying biometrics that co-relate strongly with each other. When two parameters correlate strongly, we typically need only one of them, and can ignore the other one. In such AI will continue to monitor the co-related time-series biometrics, and raise an anomaly alarm if the co-relation breaks. because there is a strong correlation. The more the AI engine drops, the easier the problem becomes, and solution is faster. The AI engine needs parameters like for each demographic of patient, what are the different types of parameters which are relevant to them, or most relevant to them. The AI engine requires one combination of parameters for certain types of patients. For another type of study, the AI engine might need a different combination of parameters. The AI engine is much more precise when the relevancy is determined and mapped. Further in that grouping/mapping, the AI engine further maps for current state e.g., awake, sleeping, exercising etc. The AI engine might also be watching for different parameters, depending on what that patient is doing. The AI engine may perform grouping of different patients based on their medical conditions, health conditions, gender, etc. For a normal patient, the AI engine monitors the similar parameters. For someone else who is pregnant, the AI engine monitors slightly different parameters. Now when someone is pregnant, and is sleeping, the AI engine drops two or three parameters, and focuses on those other parameters. The AI engine drops two or three other parameters specifically when someone is pregnant and exercising. The AI engine can work on a variety of different combinations of parameters, which become more important or less important, depending on what the patient is doing and who the patient is, and the health condition of the patient.

Reducing the number of dimensions in the data can improve computational efficiency and reduce noise in the AI system. Parameter mapping can be employed to achieve dimensionality reduction.

Closely correlated parameters can be combined using specific weights, while monitoring for potential breaks in correlation. A break in correlation will be considered as an anomaly because it will break one of the fundamental assumptions that the model is making.

Not every bio-metric needs to be tracked for every type of patient. We will create a mapping between different patient cohorts and different time series datasets. Based on prior training and subject matter expert input, we will figure out which time-series data co-relates closely with dosage of specific pharmaceutical product. c.

The AI engine takes all of these parameters into account and performs a group class parameter mapping. Then the AI models consider demographic factors like gender, weight, age, race, patient state, time of the day etc. Certain parameters become more important, at certain times of the day for certain people and certain things don't become important and the AI engine gets trained about those parameters and accordingly adjusts the weights.

The implantable device will consist of three components situated within the patient's vicinity and in close proximity to each other:
The implantable device itself, residing within the patient's body.
An external unit located near the device on the patient's body.
The Time Series Data Collection Unit, responsible for gathering information from the embedded device, the external unit, and patient input data from the mobile app.
   This data will be transmitted via the internet to the Training & Data Collection Unit, which houses various AI models.

Referring to FIG. 29B, it illustrates the AI architecture for pre-implantation training of the implantable device, following one or more embodiments. The training and configuration process for the device commences long before the actual implantation. The AI model is designed to be adaptable to various patient types and health conditions. It is fine-tuned to set cut-offs for measurements that indicate different levels of health status for each parameter. The fine-tuning process initially involves data from a group of similar patients observed in the past. These patients share similar health conditions, medication regimens, and demographics. Specific biometric sensor data from the patient is incorporated as well. During this stage, the connectivity to the device is verified, patients' medical records are reviewed, and the patient is trained to use the mobile app to provide feedback.

Referring to FIG. 29C, it illustrates the AI architecture for post-implantation training of the implantable device, following one or more embodiments. In this stage, the generative AI model generates data for emergency situations based on previous data collected, input from the patient's primary care physicians, medical history, and current vital signs. This data is then used to train the Anomaly detection model using Reinforcement Learning. In this approach, the Training & Data Processing Unit predicts future time series. Any discrepancies between the predictions and the actual outcomes are considered anomalies, and the unit adjusts its predictions accordingly. Doctors supervise the AI's learning process by providing feedback on its predictions, and the AI adapts accordingly. Additionally, the patient is provided with an external device to place on the skin near the implantable device. It contains a rechargeable battery to charge the implantable device's battery, as well as an accelerometer and voice detection module to detect falling or pain experienced by the patient. To detect sensor drift, a patient attendant will measure the patient's vitals with specialized equipment and calibrate the patient sensors if necessary.

Referring to FIG. 29D, it illustrates the AI Architecture for implantable monitoring, according to one or more embodiments. In the Final Monitoring Stage, the doctor no longer needs to constantly supervise the patient's implantable device and send new data to the Training & Data Processing Unit. Instead, a trained attendant from a Network Operating Center (NOC) will monitor the patient/device through the NOC-style web user interface if required. The software will enable attendants to monitor multiple patients, and AI will visually alert the agent if any data from the patient or device indicates malfunction or a health emergency that requires attention.

In an embodiment, 1. a web user interface for Doctors and Device specialists to enter data regarding the patient's prescribed drug dosage from the implantable device and criteria to determine health emergencies and the patient's state based on the data coming in from the patient's vital sensors. 2. This data will then be sent to the Training & Data Processing Unit (the “central brain” of the design), consists of multiple Artificial Intelligence (AI) models, including but not limited to: Generative AI to predict what comes next, Anomaly detection, and deep learning networks to process and analyze all the data that is sent to it. See #9 for more details. 3. Medical history, including data regarding the patient's height, weight, gender, race, etc. will also be sent to the Training & Data Processing Unit from the patient's medical records database. 4. Real data from a cohort of patients experiencing similar health conditions and real data from anonymous patients, as well as generated data for patient health emergencies or device malfunctions, will also be sent to the Training & Data Processing Unit through the web user interface. 5. The Patient Sensors measure the patient's vital signs such as body temperature, heart rate, blood pressure, blood glucose level, etc. This time series data will then be sent to the Time Series Data Collection Unit. 6. The Time Series Data Collection Unit is external from the patient's body and collects the time series data from the patient sensors, mobile app, and implantable device. The collected time series data is then sent to the Training & Data Processing Unit. 7. The Implantable Device contains an Embedded AI Chip that constantly collects device time series data, such as the pressure/position of the valve as well as battery level. This data is then sent to the Time series data collection unit. When the implantable device loses connection to the Time Series Data Collection Unit (the “central brain”), then the embedded AI chip will take overusing the limited functionality that it has. 8. The mobile app will take input from the patient on a regular basis, including weight, height, medication, schedule, etc. This data will then be sent to the Time Series Data Collection Unit. 9. As the Time Series Data Collection Unit reads in and stores real-time data, it will clean the data, look for patterns, identify parameters with a strong correlation and eliminate unnecessary parameters, identify time lag between medication and changes in vital signs, and accordingly recommend drug dosage amounts/times/frequency to best suit the patient. 10. After implantation, the doctor/device specialist will have access to new data on the web user interface regarding the device operation status, reinforcement training, supervised training, and historical data classification. 11. The patient will also be given an external device to place on the skin, which contains a rechargeable battery and will charge the implantable device. It also contains an accelerometer and voice input module to detect if the patient is falling or in pain. 12. When the Training & Data Processing Unit makes drug dosage recommendations, they will be sent back to the implantable device to be administered. 13. A patient attendant will monitor the patient's vitals and calibrate the sensors if sensor drift is detected. 14. In the post-implantation training stage, the Reinforcement Training and Supervised Learning will play key roles. After detecting patterns in the patient's and device's data, the Training & Data Processing Unit will make predictions about future time series. If the outcomes don't match up with its predictions, that will be considered an Anomaly, and the Training & Data Processing Unit will adjust its predictions accordingly. 15. In the final monitoring stage, the doctor will no longer be frequently supervising the patient's data through the web user interface. However, help desk attendants at a Network Operating Center will have access to that same data and can make any changes through the web user interface when necessary. 16. The NOC-style web user interface will allow help desk workers to access data for multiple patients and check on one of the patients when needed (for example, if the AI detects a health emergency or device malfunction).

### Anomaly Detection:

The AI engine performs predictive modeling for Anomaly Detection. The AI system can predict clinical outcomes for a specific patient based on hypothetical parameters provided by the patient's doctor or care provider. These input parameters may include drug delivery schedule, drug dosage, drug type, and drug delivery device type. A user interface, such as a web-based application or smartphone app, allows the care provider to input these parameters. The AI's trained prediction model processes the inputs and provides the user with predicted clinical outcomes, side effects, and other behavioural or physiological changes expected for the patient. This allows physicians and care providers to assess various drug delivery schedules and device configurations in a controlled, low-risk manner before administering a new treatment regimen. Furthermore, the model can detect anomalies and raise alarms if predicted outcomes deviate from actual clinical outcomes, thus enhancing patient safety.

In another embodiment, the implantable device having the artificial intelligence performs anomaly detection. The anomaly detection comprises at least one of detecting sensor failure, detecting device failure, detecting severe reaction in the patients, communication failure between the components, battery power drain, etc. The implantable device detects the sensor failure by monitoring the data received from the sensors. The sensors may include the sensors of the implantable device, sensors associated with the external devices, sensors that are affixed to the patient's body, sensors that are in communication with the implantable device, etc. In one embodiment, the implantable device detects that there is a sensor failure, when the implantable device does not receive data from the sensors within a scheduled time period. In another embodiment, the implantable device detects that there is a sensor failure, when the implantable device receives bad data from the sensors within a scheduled time period. The bad data may be the data that comprises unusual readings/irrelevant readings. The artificial intelligence engine may be initially trained with right data sets for each and every sensor and each and every scenario (including diabetes, high blood pressure, low blood pressure, glucose, brain tumours, prostate cancers, etc.). The artificial intelligence learns from the initial data sets and gets trained. The artificial intelligence engine may also be trained in a simulated environment/supervised environment in which case the artificial intelligence engine learns and improves further. Further the artificial intelligence engine may also be trained with respect to readings and/or sensor data from animals. In this case, the artificial intelligence engine correlates with the readings of the animals with the patients and learns and improves further. In one embodiment, the above training fed to the artificial intelligence engine enables the artificial intelligence engine to perform the anomaly detection.

The AI engine detects the device failure such as battery drain, high battery voltage, low battery voltage, transmission failure, reception failure, internet connectivity failure, etc. The AI engine monitors the battery parameters for a predefined time and plots a curve. The AI engine may be pre-trained with the battery parameters. The AI engine then detects for an anomaly in the curve (e.g., sudden rise or fall in the curve) and classifies those as an anomaly. The AI engine may consider the lifespan of the battery while detecting an anomaly. In one embodiments, the AI engine aloe monitors upon an input for a predefined time upon raising a request or an enquiry. The AI detects the anomaly when there is no input for the predefined time period upon raising the request or the enquiry.

### Supervised Learning:

Supervised learning algorithms can be used after historical data has been classified by human operators as normal or anomaly. Example of such algorithms like:
Support Vector Machines (SVM)
k-Nearest Neighbours (k-NN), and
Random Forests

### Recurrent Neural Networks (RNN's):

Recurrent Neural Networks (RNN's) like Long Short-Term Memory (LSTM) and Gated Recurrent Units (GRU) can be used to capture long term dependencies in complex temporal patterns and predict the output in real-time. If the actual output defers from the predicted output more than a specific threshold, then it could be considered an anomaly. Autoencoders are commonly used for this purpose. These neural networks can be trained for all-time series data inputs from the device and patient biometrics in parallel.

Classification-based approach: Human operators identifies acceptable and unacceptable combinations across all-time series data sets. After identifying the lag, the lag is applied to adjust the time-series data sets to line them up. The neural networks is trained to classify data points or sub-sequences as normal or anomalous based on human classified data sets that have been lag adjusted.

Autoencoders and Variational Autoencoders (VAEs) can also be used for unsupervised anomaly or fault detection.

Time-Series Clustering: AI models can use clustering techniques, such as k-means, DBSCAN, or hierarchical clustering, to group similar time series together and identify outliers based on a defined distance metric.

Change Point Detection: AI can identify points in the time series where the statistical properties, such as mean or variance, change significantly. Methods like CUSUM, Bayesian Online Change Point Detection, and window-based approaches can be employed.

AI models can be designed to adapt to new data points continuously, allowing them to detect anomalies in real-time.

### Outcome Based Dosage Adjustment:

### AI System Overview and Therapeutic Benefit Analysis

The present invention relates to an artificial intelligence and machine learning system (AI) that monitors the effectiveness of drug delivery and optimizes treatment for patients. The AI processes drug delivery device usage and drug delivery data aggregated with clinical outcome data, such as self-reported symptoms and sensed data, to determine the therapeutic benefit of a drug and the occurrence of side effects. By analysing correlations between specific drugs or drug delivery schedules and certain side effects, the AI can tailor treatment plans to individual patients. This helps to develop new drugs or drug delivery regimens that produce desired clinical outcomes for a patient population.

### Sensor Data Integration and Surgical Outcome Analysis:

Sensor data from implanted and external devices can be fed to the AI engine for analysis of clinical outcomes (patient biometric data) and control of drug delivery. In some embodiments, this can include post-operative outcome analysis, with the sensor data informing the analysis of drug and surgery effectiveness. The drug delivery to the patient can be controlled before, during, and after surgery, with sensor data informing when to deliver the drug for maximum effectiveness. This integration of sensor data allows for a more comprehensive assessment of the patient's treatment and contributes to an optimized drug delivery strategy.

### Implantable Device and Dynamic Dosage Adjustment:

In one embodiment, an implantable device can dynamically alter drug dosages based on the patient's current health condition. The implantable device may include an artificial intelligence engine integrated within the device or located in proximity to enable communication. In another embodiment, the AI engine may reside remotely, communicating with the implantable device via the cloud. The AI engine can adjust drug delivery dosages based on the patient's demographics, current state (e.g., sleeping, exercising, awake), and health condition, using inputs from sensors (e.g., biosensors), medical records, and patient feedback. Additionally, the AI engine can select a specific drug among multiple drugs within the implantable device based on the patient's current health condition.

### AI Engine Training and Personalized Recommendations:

The AI engine undergoes two aspects of training: training on a variety of different cohorts (e.g., race, ethnicity, medical condition) and training on specific medical conditions. This extensive training process enables the AI engine to make more accurate predictions and recommendations for patients. In one embodiment, the AI engine recommends a specific pharmaceutical product for the patient, and in another embodiment, it fine-tunes the dosage specifically for the patient in a supervised learning process. The AI engine also accurately detects anomalies in implantable devices, sensors, and external devices, ensuring that any issues are promptly addressed and resolved.

### Feedback Loop and Data Monitoring:

A feedback loop is used by the AI engine, receiving input from patients, sensors, and external devices. Time series data from various sources, such as patient data (e.g., oxygen levels, blood pressure, body temperature, motion sensors, insulin levels) and device data (e.g., battery condition, voltage, pump).

### Device Specific Calibration:

The device specific calibration comprises Calibration and Fine-tuning of the Implanted Device for Enhanced Precision and Anomaly Detection.

### Pre-Implantation Calibration and Device Testing:

Before implanting the device beneath a patient's skin, it is essential to perform rigorous testing and calibration to account for minor variations due to manufacturing discrepancies, battery conditions, and changing conditions of the osmotic membrane. The AI engine can learn the manufacturing specifications and make adjustments as needed to optimize the device's performance and minimize false positives.

### AI-Governed Sensor Management:

The AI engine should be capable of handling sensor-related issues, such as sensor failure, sensor drift, and data inconsistencies. By continuously monitoring the data received from sensors, the AI engine can detect minor drifts and adjust the time series data accordingly.

### Real-time Sensor Drift Analysis and Adjustment:

The AI engine should analyze the sensor data in real-time and identify drift patterns that might impact drug delivery accuracy. The system can then make necessary adjustments within a predefined tolerance range to ensure optimal drug dosing. Beyond this range, the AI engine would determine that the sensor has failed, triggering an anomaly detection process.

### Anomaly Detection and Prompt Intervention:

In the event of sensor anomalies or data inconsistencies, the AI engine should have a robust mechanism for detecting and addressing these issues. This ensures that any problems are identified and resolved promptly, safeguarding patient safety and optimizing treatment outcomes. The AI engine fine tunes the Implanted Device for Patient-Specific Drug Delivery and Anomaly Detection.

### AI-Driven Personalized Drug Delivery:

By analyzing individual patient data, the AI engine can customize drug delivery based on the patient's current state, such as their activity level or sleep patterns. The AI engine automatically considers individual patient differences, such as metabolic rates, specific health conditions, and lifestyle factors, further optimizing drug delivery and efficacy tailored to each patient.

### Dynamic Dosage Adjustment and Timing:

The AI engine is capable of adjusting both the timing and dosage of the medication according to the patient's specific needs. By learning the most effective state for drug administration, the AI engine can wait for the ideal moment, such as when the patient is sleeping or after they have eaten, to deliver the medication.

For example, if the patient is exercising and when the dosage happens, the blood moves faster, and the heart pumps faster which goes through the body more rapidly and the patient might have a different reaction compared to when the patient is sleeping and when the dosage happens when sleeping. The AI engine learns when to choose the time for the patient to provide dosage.

For example, the AI engine may learn that the sleeping time is the right time to provide appropriate pharmaceutical drugs. The AI engine then detects the sleeping state of the patient. The AI engine may, with the help of a motion detector built in, determine the sleeping state, and instruct the implantable device to deliver the appropriate drug at the appropriate time.

The AI engine performing the dosage adjustment is not just adjusting the amount of dosage, but also the time the dosing is actually delivered by the implantable device. The AI engine is capable of adjusting both the time and amount of the dosage.

### Anomaly Detection Through Correlation Analysis:

The AI engine analyzes datasets received from the device and user feedback to identify potential anomalies or irregularities. By correlating device data with user-reported symptoms, the AI engine can detect issues early on, allowing for timely intervention and medication adjustments.

### Long-Term Monitoring and Drift Detection:

The AI engine continuously monitors patient metrics over an extended period to ensure that they are progressing in the expected direction. This long-term monitoring enables the detection of any drifts in patient health, allowing for proactive adjustments in medication dosing and timing. This comprehensive approach to anomaly detection ensures optimal patient outcomes and overall treatment efficacy.

In an embodiment, the AI engine analyses the datasets received from the device and the feedback from the user. For instance, if the user starts having some symptoms. The AI engine performs correlation between the datasets/feedback from the device and the datasets/feedback from the user to detect the anomaly. For example, when the AI engine instructs the implantable device to provide dosage to the patient, the problems might not start right away, the problems could start after a week or maybe two weeks, three weeks. The caregiver observes a drift in certain patient metrics. The caregiver may observe those drifts over a period of time. The caregiver detects that the patient is not adjusting the way as expected them to adjust. The implementation of the AI engine in the implantable device detects that there is drifting away in the wrong direction in a first spike as early as possible leading to reschedule or re-adjust the medication as needed. The AI engine also monitors the metrics over a period of time, and determines if they're moving in generally expected direction. The AI engine monitoring the metrics (not just observing severe reaction at the moment, but also observing metrics over a period of time) is important in the anomaly detection.

FIG. 28D shows a system according to one or more embodiments. The system comprises a receiving unit, an implantable device, and an external unit. The external unit comprises the external sensors. The receiving unit may be the unit in the AI engine. The implantable device may have a bunch of sensors. The sensors could determine X number of metrics. The implantable device communicates with the external unit. The external unit comprises other types of sensors (like a motion sensor). FIG. 29D shows the device, the external unit, some sensor data coming from the device, some sensor data coming from the external unit, which is interfacing with the implantable device. The data sets may be going into the implantable device as time series data. The device data is something that we are observing on a regular basis. The data coming from the patient, which is patient data, could be from the external sensor, or it could be from other places integrated with the system as well because the whole system could be integrated. For example, the sensor on the finger measures the oxygen level. The data point (e.g., oxygen being measured) obtained when the patient sleeps could be a problem and that could be another data point that could be entered in. The system comprises a health data anomaly detection system, which allows to have a variety of different time series data, which can be plugged in not just what is coming from the device, or coming from the sensor, or coming from the patient. The system also enables the user to plug in another sensor in addition to the integrated system. The AI engine, depending on how many different time series data, determines the offset of the patient data and the device data. Based on the offset detection, the system the AI engine determines the anomaly. The AI engine is trained for offset.

The AI engine is a pre-trained AI engine on patient demographic like Age, Race, Gender and other health conditions etc. The AI engine takes information in order regarding parameters of the device, and a variety of different data points coming in. But then the learning by the AI engine could depend and could also change based on the offset between the time series data coming from the patient and time series data coming from the device. The AI engine also learns over time, depending on the current state of patient, medical condition, and rest of the time-series data sources (as defined above). The AI engine undergoes on the fly training, like patient specific training, variations in the device. The AI engine then learns more about the patient, their habits and what they do on a regular basis. The AI engine then based on the learnings undergone start triggering anomalies, and alerts. The AI engine also gets feedback from a human being. The AI engine may provide different types of alerts such as a medium level alert, a low level alert and a high level critical alert. For low level alerts, The AI engine notifies operators to come in and observe the data set, and then further train the AI. The observer determines whether the alert is a medium level alert, a lower level alert, or a higher level alert. The operator may provide a customized training, which is happening on the data. The operators may provide the training on a regular basis, not real time. The operators would go with low level and medium level alerts and look at that data, and further customize and clean the datasets for that patient system in order to train the AI engine. In one embodiment, the AI engine customizes the data based on the anomaly detected with the cohort of the patient and learns from the datasets associated with the other patient in the cohort. Every patient might be different and have slightly different metrics. The AI engine makes use of the help desk center (e.g., network operating center) in the telecom space. The help desk center observes the datasets on a regular basis by human beings, and then they are further altered and worked in.

The AI engine can apply clustering algorithms like k-means, DBSCAN (Density-Based Spatial Clustering of Applications with Noise) on, dosage data, patient state and patient bio-metric data to group datasets in a several multi-dimensional clusters space together. Any data points occurring outside these multi-dimensional cluster shapes is considered a potential anomaly. Prior to sending data to the clustering algorithm, the data would be adjusted for lag between cause and effect. These multi-dimensional clusters can be pre-created based on training data from several devices and patients. Subsequently these shapes will be trimmed/adjusted to patient demographics (age, gender, race, medical condition etc.), and then in last step further trimmed and adjusted for the specific patient. What is an anomaly for a regular patient with similar characteristics might not be an anomaly for specific patient depending on their group of medical conditions and medications that they are taking. In k-means algorithm Anomalies can be identified by analyzing the distance between each data point and its corresponding cluster center. For DBSCAN, Points that are not part of any cluster are treated as noise, and in this context, they can be considered anomalies. The AI engine initially may not know how many clusters you need to cluster. The AI engine learns and improves further in real time and/or in a simulated environment to kind of have to experiment with a bunch of different numbers to figure out what is the most optimal number of clusters.

The AI engine may receive the time series data in a multi-dimensional space right. For example, the AI engine may receive the time series data in a representation (for example, in a two or three dimensional space). The datasets received in a multi-dimensional space of data are constantly being read by sensors that are outside the device or inside the device. The datasets read includes the patient data and/or device data. The AI engine learns, in order to detect anomaly, within what range all of these data sets the sphere is created, which is the boundary which could be a sphere. The AI engine also learns, based on the datasets received, what is the shape within which every datasets falls. The AI engine, upon learning the multi-dimensional shape of the cluster, determines an anomaly when a dataset goes outside that shape. The shape drawn is then highlighted for anomaly detection. The AI engine learns the boundary and looks for data within that space and/or the boundary. For example, for a specific patient, this shape could be different. For the patient already having a heart issue, the heart will function differently, and datasets will be different. When the pre-trained AI engine is implemented the accuracy of functioning of the AI engine is not optimized, because the AI engine is initially trained on different devises and different patients (similar in nature, but nevertheless not the same patient). But for that specific patient, who is having a heart issue, the AI engine needs to be retrained and adjusted. The datasets received and the plot made for that specific patient may have a shape that looks slightly different. The shape of the specific patient, in that multi-dimensional space, might mostly fit inside the space of the healthy patient. There could also be certain points coming from the regularly planned shape (e.g., based on the trained person). The AI engine learns the above in a few iterations. Upon learning the above, the AI engine in the case of that patient, certain cases, few datasets normally might not actually be an anomaly. The AI engine may then use that patient as a base patient in that cluster or category. The AI engine creates the cluster properly over time. In one embodiment, the AI engine is trained in such a way before implanting or put into action. The cluster then gets recorded over the network. Once the AI engine creates the clusters properly and settles the datasets, the AI engine accuracy improves and will not provide any more abnormal alerts.

The other issue that the AI engine might be facing is false positives. So, for false positives, the system reduces the number of false positives, because if the device keeps shouting out alerts, every few minutes, when the real one comes in, the patient might ignore it. So false positives are not good. In any such scenario where alerts are coming in, the patient's feedback would be helpful in reducing the false positives. The patient's feedback can be thumbs up or thumbs down which states whether they're feeling okay or not respectively. The data point, which is patient feedback, is not just from devices, but the patient (i.e., the human being) can respond back to the system. The patient feedback trains the AI model further. For instance, the device attached to the elbow, or shoulder has a voice module device. The patient can provide feedback via the device. The device might beep and then ask a question. The device might further beep, and then the patient can tap it to provide the input. The patient may also double tap to stop providing, because they are in a meeting.

The device may ask a series of questions. The patient may then make a single tap for “Yes”, or two taps or “No”. The patient feedback can be provided via the device associated with the user without the need of going back to the system. The voice module is an interesting data feed for the system. The voice module allows us to read that data, they are easy to put in, and then train on. A voice module could be helpful for a lot of these patients, sometimes if elderly living alone. The voice module can detect when the patient shouts. The voice module further detects that could be a stress in the voice itself, and then takes it as a patient input. So patient feedback could be coming as direct from the voice module from surroundings and a variety of ways that data could be collected. The voice module just senses inflection instead of the actual content. The voice module does not require to actually interpret the voice except for us indicators. The voice module figures out if the voice is stressed or determines that someone is shouting for help.

The AI engine may be combined with three axes e.g., inputs from accelerometer sensor, biometric sensors, and voice module. For example, when the patient falls down and they are shouting for help. The AI engine records different inputs, like a sudden fall input from the accelerometer, a voice input from the voice module and sensor inputs like heart rate from biometric sensors. The different inputs from different axes get correlated by the AI engine and provides training to the AI engine.

The AI engine may undergo two types of training. The first is initial training (pre-training), and subsequent (second) re-training to fine tune the AI for the specific device and specific patient. In the pre-training the AI engine learns to understand the group of the patient. The patient group may be categorized based on the demographics. The AI engine is a trained device initially for different categories for example, kids, men, women, boys, girls, older people, etc. The AI engine undergoes different types of training in particular categories based on the current state e.g., sleeping, awake, exercising, eating, etc.

The AI engine first detects the state. The AI engine comprises a state detection module which detects what state the person is in. The state detection module depending on time of the day and various other metrics, figures out the current state such as sleeping, or awake. Based on the current state, the AI engine instructs the implantable device to provide dosage and/or dynamically adjust dosage at appropriate times. The AI engine comprises a group identification module that determines which group or category the patient falls in. The AI engine may also perform lag detection between the data of the device and the patient. The AI engine may also monitor how the patient is performing over a longer period of time. The AI engine is capable of receiving medical conditions from the patient feedback. In one embodiment, the AI engine extracts the medical conditions (e.g., patient undergoing medications) from the previous medical records. There might be different medical conditions and the AI engine learns more about the medical conditions and the corresponding treatments/medications provided. For example, for a heart patient the AI engine defines medical conditions and determines what the cohort for that patient. The AI engine also gets trained regarding even the approvals coming from FDA approvals only for a specific type of patients having specific conditions and things. The AI engine also learns about the datasets based on which have been trained for that specific conditions. The AI engine also gets trained on other types of medical conditions that have enough patients.

The AI engine gets trained in three types: supervised learning, unsupervised learning, deep networks, and large language models. Supervised Learning comprises labelling. As the system comprises a data set, supervised learning is a labor intensive, costly process, where one has to go through the data set, start labeling everything coming from it. The dataset has to be read and marked as anomaly or not an anomaly. The supervised learning happens for a long period of time for a lot of data sets. The AI engine eventually starts learning and figuring out the labeling. The second one is unsupervised learning. Unsupervised Learning is nothing but clustering which is another option. And the third option is deep networks. The AI engine starts using deep networks. And the fourth one, within that deep network classification coming in is large language models, which are becoming popular, (for example: ChatGPT®). The AI engine uses the large language models in detecting the background noise. The large language model can understand the voice data from a microphone, and transcribe it to understand content and tone of the voice. The model can also detect unusual noise, calls for help, and sense anxiety, stress or distress in the voice of patient. The voice inputs may be received from the microphone. The Large Language Model can classify background noise with voice inputs. start classifying the background noise and extracts the voice input.

The AI engine after dosing, monitors some other parameters based on recent case study or recent medical scenario. The AI engine upon monitoring certain other parameters, which become more important based on the recent medical scenarios. The AI engine keeps monitoring the regulations and/or recent updates in the medical field may monitor for other parameters dynamically after the dosage is done. The AI engine may communicate with an excellent medical database that keeps track of the recent updates in the field. The AI engine may comprise a natural language processing module that interprets the context and provides recommendation to the AI engine. The mapping also might become fairly important for us down the line. The AI engine tracks and updates the mapping dynamically to keep the changes up to date.

The AI engine comprises a system that allows to plug in any kind of time series dataset, and then start training the AI engine quickly. The AI engine enables any kind of time series data to be fed into the system. The AI engine comprises visual interfaces for various data points. The visual interface is one where the different data points can be plugged in. The visual interface is also adapted to read all of these datasets, and constantly train the AI engine on a large number of patients. The AI engine can then be specifically customized for a specific patient. The AI engine also works and functions when few data points are removed from that patient. For example, the patient has a sensor on their arm, and while sleeping the oxygen sensor falls off in your sleep. The patient may not want an alarm to go off and notify that the oxygen sensors are off. The AI engine works even in such a scenario when some of these time series datasets flatten out.

The AI engine also functions and provides anomaly detection and/or dosage adjustment when the device flats out or certain device sensors stop functioning. The AI engine functions in a manner where even if certain data sets are not available, or if additional data sets are available, the AI can adjust and continue to make decisions. It can replace one parameter with another strongly co-related parameter/time-series. The AI engine is able to deal with certain critical scenarios. For example, the AI engine has that data point constantly when a patient is on a ventilator. But if there are other sensors plugged into the patient, and if you have that data coming in, the AI engines take in that data as an additional data feed and incorporate that. The system starts training based on that additional data feed, and then starts learning based on that data and then starts detecting based on patient/agent/care provider feedback, or dosage details. The AI engine performs the anomaly detection based on additional data as well. In one embodiment, the additional data can be generalized. In one example, the feedback for the age of the patient may be specific, and it cannot be generalized as much, because feedback for that age has to be specific based on the person. The AI engine regarding the dosage part has to be pre-trained through and customized based on the recommendation of the doctor, and allows the fine tuning of the dosage of the patient to take place remotely. The fine tuning may happen through a telemedicine operation where patient is talking to operators, nurses and doctors and providing their feedback. The doctor may provide instructions via the user interface to the AI engine. The AI engine then observes and sees what's happening to the patient by monitoring the sensors. In one embodiment, the doctors may also manually observe the readings. The AI model could be an incremental model where via the user interface the dosage can be adjusted dynamically during these particular events. In one embodiment, the AI engine itself upon monitoring steps up the dosage. For example, the AI engine steps up 10%, your dosage, if these events are detected.

The AI engine might work under different conditions. The AI engine is enabled to automatically choose the dosage. The AI engine until it learns the dosage the patient/the caregiver/the doctor could start off with medications, like antibiotics by providing instructions via the interface. The AI engine also learns that certain antibiotics have stepped dosage requirements based on recent updates in the external medical database through NLP. The AI engine may also receive approval when stepping up the dosage requirements from the operator. The AI engine might also incrementally increase the dosage and observe if the patient is reacting or not reacting to the dosage.

The conventional device does not have the capability of dynamically monitoring and testing current dosage and dynamically monitoring the current state and the health condition of the patient which leads to the illness like kidney failures. The present device with the AI intelligence having the capability of dynamically monitoring the current health condition, the current state in response to dosage enables to dynamically alter the dosage as and when needed which provides accuracy in treating the patient. The present system constantly monitors the patient and if something is going wrong, the AI detects what is going wrong before the person's kidney fades, or before that person's heart fails. The present system is a zero failure system where nobody has a bad reaction, like a kidney failure or any failure because the AI engine detects before that can even happen.

The implantable device can be useful for the patient where infusions are happening or for a period of time, not just treatment. The device implanted within the patient can provide notifications regarding the health condition dynamically and dynamically alter the drugs, dosage, and medication timing accordingly to provide a healthier and longer life. The implantable device having the AI engine constantly checks the body and using artificial intelligence makes the decision based on real parameters obtained from that particular individual. The implantable device provides an extreme personalized medication.

The AI engine comprises a lot of intelligence which may require communication between the AI engine and the implantable device and other external devices and sensors through network connection, internet connection or other technology. When the connection fails or other technology fails (e.g., internet connectivity failure, Bluetooth® connectivity failure etc.), the communication gets interrupted, it is too late for the patient to get the dosage or change in the dosage or timing. The present system uses embedded Artificial Intelligence to overcome the above drawback. The AI is embedded on the sensor sitting on the top of the sensor.

The embedded AI enables data reduction, which essentially means that not all of the data is being sent to the cloud. The embedded AI enables to perform a lot of analysis happening locally, as much as possible. The embedded AI enables reduction of the amount of data that we are sending out, and only relevant data is sent out. The embedded AI also protects personal privacy enabling cybersecurity. The embedded AI also reduces the number of data extracted from the patient. In an embodiment, the system still be able to make the same decisions that we are able to make on this AI. But it should be configurable from the cloud. The operator analyses the data, changes it, configures and deploys it for the implantable device. The AI engine then changes the way it was thinking where it is residing or acting. The functioning of the AI engine, in this instance, happens from the cloud. The configuration has to be available in hands of a trained technician or trained operator who is closely monitoring the patient.

FIG. 28E is a block diagram of an example device 2870E that may be used externally of a patient's body, and that may communicate with an implantable medical device, in accordance with one or more implementations. In some examples, device 2870E may correspond the external device 2880E. Device 2870E may be used to receive data (e.g., physiological signal data) from an implantable medical device, where the data may be communicated from the implantable medical device to the external device 2870E over a Galvanic communication link, and device 2870E may then either analyze the received data locally at device 2870E or may transmit the data received from the implantable medical device to another device external of the patient, such as a cloud connected device.

The external device 2870E includes a processor 2884E, which may include processing power sufficient to provide machine-learning algorithms, artificial intelligence algorithms, or both machine-learning (“ML”) algorithms and artificial intelligence (“AI”) algorithms to process data received from the implantable medical device (i.e., such that those algorithms may run locally on the external device 2870E). For example, the external device can include ML and AI algorithms implemented locally in the processor 2884E. The ML or AI algorithms may, in some implementations, learn patient-specific characteristics over time, and may use the learned characteristics to optimize detection and prediction of events. In some implementations, the ML or AI algorithms may use patient activity data (eating activity, sleeping activity, exercise activity, and the like) and related information (e.g., time of day that the activity occurs) to optimize detection and prediction of events. In some implementations, the ML or AI algorithms may use patient input of occurrence of clinical events (for example, syncope, myocardial infarction, cardiac arrhythmias, stroke, or acute heart failure) to optimize detection and prediction of events. In some examples, the local ML and AI algorithms at the external device 2870E may be revised or updated from time to time based on ML or AI algorithms performed on one or more other computing devices, such as cloud connected computing devices, where the one or more other computing devices may provide the revision or update to the external device 2870E.

The external device includes memory 2886E (SRAM, flash, DRAM, or other types of memory) where data and instructions can be stored, for example. The external device 2870E includes a battery/power management module 2887E, which includes one or more batteries and circuitry to monitor power management for the external device 2870E. In some examples the power management module can provide a battery status to the user. The one or more batteries may be rechargeable, and in various implementations may be recharged by a base station, by a power cord, or by other recharging techniques. In implementations where the implantable medical device includes a rechargeable battery, the battery/power management module 2887E may be used to recharge the battery of the implantable medical device, according to some implementations.

The external device 2870E includes a Galvanic communications module 2888E can be used to communicate via a Galvanic communication link with another device, such as an implanted medical device. For example, physiological signal data stored at the implanted medical device may be uploaded via a Galvanic communication link to the external device 2870E, where the data may be analyzed at the external device 2870E, or in some cases transmitted to another device, such as a cloud-connected device, for analysis. In some examples, the external device can use the Galvanic communications module 2888E to send commands, markers, updates, or revisions to the implant. The Galvanic communications module 2888E can include a transceiver (e.g., a transmitter and a receiver) capable of Galvanic communications, and two or more communications electrodes 2889E, which may be on a bottom outside surface of the external device for contact with the skin of the patient. As described above, more than two communications electrodes 2889E are possible.

The external device 2870E includes a long-range telemetry module 2890E with an antenna 2891E (e.g., an RF antenna) that can be used to communicate with other devices. The long-range telemetry module 2888E includes one or more transceivers that can communicate using one or more RF telemetry communications protocols, such as BLE or MICS. For implementations of the implanted medical device that include a long-range telemetry module at the implantable device, the external device 2870E and the implantable device may communicate via RF telemetry (e.g., via BLE or MICS). In various implementations, the external device 2870E may also communicate with other devices, such as a smartphone, a wearable smart device, a base station, or other devices via the long-range telemetry module 2888E. Long-range telemetry communications can be useful for sending smaller data files and alerts that can be received real-time, rather than waiting for a Galvanic data exchange, which may be scheduled to occur at predetermined periods (e.g., daily, weekly, monthly, or another predetermined period). Alerts may be sent to the patient or to the physician, according to some implementations.

A connectivity module 2892E may be used to communicably connect the external device 2870E1 to one or more other devices via Wi-Fi, wired internet, mobile data, or other connectivity techniques. For example, the external device 2870E may communicate or communicably connect with one or more cloud-connected devices using the connectivity module. In some implementations, the external mobile device may upload to a cloud-storage location data previously uploaded from the implantable medical device to the external device 2870E1. In some examples, the ML or AI algorithms executing locally at the external device 2870E to process patient data can provide data or results that can be sent directly to the patient (e.g., to the patient's smartphone or computer) or to a clinician via the connectivity module 2892E.

As will be described in more detail below, one or more cloud-connected devices, such as one or more super-computers, may process patient-specific data, including data captured by the implantable medical device, and may perform ML and/or AI algorithms in analyzing the data. The data may include very large sets of data captured over long periods of time, such as data captured continuously by the implanted device over a long period of time. In some examples, one or more such devices may process data provided from the implantable medical device (or other data), whether received directly from the implantable medical device or alternatively from the external device 2870E, and may provide feedback to the external device 2870E to optimize performance of local ML or AI processing at the external device 2870E.

The external device 2870E includes various I/O devices that can be used to receive information from a user or provide information to a user. A microphone 2893E may be used to receive voice commands, voice inputs, or sound inputs at the external device 2870E. In some examples, voice commands, sound environment recordings, breathing sounds, heart valve sounds, and other respiratory sounds such as cough, wheezing, labored breathing, pneumonia, and the like, may be recorded. In various implementations, voice commands can be processed and or stored by the implantable medical device or by the external device 2870E. In some examples, the implantable device can communicate a voice command to the external device (or vice versa) by long-distance telemetry (e.g., via MICS or BLE), and the external device 2870E may process the voice commands, or in some cases communicate the voice commands to a cloud connected device (or other device) via the connectivity module 2892E.

An audio output module 2894E can include one or more of a speaker or buzzer, for example, and can provide one or more of beeps or voice commands, according to various implementations. In some examples, the audio output module can provide features such as range finding when establishing a Galvanic communication link (e.g., by providing audible feedback so the user is aided in optimally positioning or aligning the external device to communicate with the implantable medical device.

A visual output module 2895E can include one or more light-emitting diodes (“LED's”) that can provide status information. The one or more LED's can provide a status indication of, for example, external device battery life, implantable medical device battery life, galvanic communication link, faults, or status indicators, such as patient health status (e.g., presence of AF, arrhythmias, or no health concerns detected), to list just a few examples. The visual output module 2895E may also include a graphical user interface (“GUI”) with an LCD screen (e.g., graphic text, black/white, or full color LCD), according to some implementations. The LCD screen may be a touch screen receptive to touch inputs in some implementations or may provide soft keys for patient input or clinician input. The GUI can provide feedback and information to the patient on their health status, instructions from the physician, and cloud-based automated directions that may be prescribed by the clinician, according to various implementations. In some examples, patient input may be received via the GUI, including, for example input related to observed health conditions, such as a syncope event, dizziness, or not feeling well, or for entering information such as a daily activity log relating to activities such as eating, sleeping, exercising, or compliance with medication requirements.

The external device 2870E can include one or more sensors 2896E, such as an accelerometer sensor or piezo element, temperature sensor, a weight measurement sensor, or a snoring sensor. The one or more sensors 2896E may be used to detect and record the patient's body position, motion, or alignment of the external device 2870E relative to the implanted device, for example, or to record the patient's temperature, weight, or indications of snoring.

The external device can optionally include a surface ECG measurement module 2897E, and auxiliary ECG electrodes 2898E to record a surface ECG signal. In other implementations one or more of the electrodes 2898E may be included on the bottom external surface of the external device 2870E.

In various implementations, the external device 2870E may record, using the surface ECG measurement module 2897E and auxiliary ECG electrodes 2898 a patient's ECG signal and may compare it to the ECG signal recorded by the implantable medical device. In various implementations, the external device 2870E may use such comparisons to detect issues with the implantable medical device, such as with the ECG measurement circuit of the implantable device. In some examples, the surface ECG measurements obtained by the external device 2870E may have multiple vectors, which may provide greater diagnostic ECG data. In some examples, more than two auxiliary ECG electrodes 2898 can be used. For example, three electrodes 2898 (or more) can be used in some implementations to provide two or more vectors. In some examples, a first vector may be orthogonal to a second vector. Auxiliary ECG wires can be used (e.g., by the patient) to record a much wider vector than would be possible for electrodes on the surface of the external device 2870E. A potential benefit of having multiple ECG surface electrode vectors and wider vectors may be that a larger volume of ECG information may be collected for the patient, which may enable deeper levels of analysis in some cases. This may be helpful, for example, in cases where there is concern about the patient's condition, or where more diagnostic data is needed. In some examples, such additional ECG recording may be performed to maintain a patient trend. In some examples, such additional ECG recording may be performed to compare with results from the implantable ECG monitor.

### 3. Use of AI for In-Vivo Device Calibration

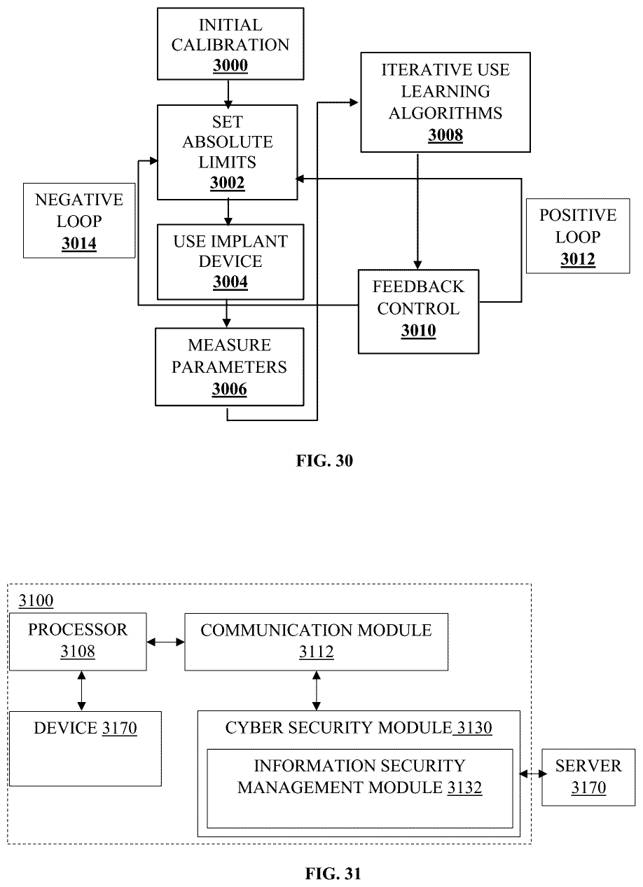

FIG. 30 shows an example flow chart of device calibration processes according to some embodiments or methods of the present disclosure. In stage 3000 the device is calibrated based on the treatment site, type of drug, and an indication to the implantable device to evoke the correct treatment protocol. Furthermore, the subject may set personal data such as comfort level. The limits and parameters defined in stage 3000 are set in stage 3002 and incorporated into the implantable device. In stage 3004, the initial treatment is implemented by a microcontroller and by at least one or more sensors, which may be used to monitor the advancement of the treatment process as the treatment protocol advances. In stage 3006. The measured parameters including, but not limited to, dosage timing, timing of resting period, length of resting period, heat levels, temperature, power range, and the like, are measured and may be altered to bring about the effects relative to the elapsed time and where treatment effects such as biochemical levels or threshold levels of the subject are relative to expected levels based on the predicted treatment effect. In stage 3008, the treatment may be altered according to a learning algorithm including, but not limited to, a PID (proportional-integral-derivative) controller, artificial intelligence mechanism, or the like to adjust or adapt the treatment protocols to be more specific or tailored to the drug delivery needs of the subject. In stage 3010, the altered treatment according to the learning algorithm is tested using feedback control that may be repeated in earlier stage 3002, which may include positive feedback 3012 for certain parameters or negative feedback 3014 for other parameters. Positive and negative controls are used to reset and alter old protocols and may be used to adjust new parameters or treatment protocols for future use in stage 3002. Different treatment protocols may be stored by a database 3004 communicatively coupled with the implantable device, for different situations. The calibration protocol depicted above may be implemented one time for a specific subject only using stages 3000-3004, while all the stages may be used when treatment is implemented once a day, at every drug delivery event, or in a dynamic process, as necessary. For example, the algorithms based on data for example, blood glucose readings, dosage information, user input, etc., can positively control the drug delivery if the average blood glucose concentration continuously remains at higher than a predefined level for a predefined time period. In another example, the algorithms based on data, e.g., blood glucose readings, dosage information, user input, etc., can negatively control the drug delivery if the average blood glucose concentration continuously remains at lower than a predefined level for a predefined time period. U.S. Ser. No. 10/653,836B2, incorporated with this specification in its entirety, describes a method for monitoring and controlling the delivery of medication via a delivery device.

In an embodiment, the implantable device can deliver a drug in multiple delivery modes. The multiple delivery modes can comprise, for example, a closed-loop delivery mode and an open-loop delivery mode. During a closed-loop delivery mode, the implantable device may dispense medicine according to an automated control algorithm that adjusts the medicine dispensation rate in response to sensor feedback (e.g., a blood glucose sensor) or other feedback, whereas in an open-loop delivery mode the implantable device may dispense medicine based, at least in part, on user input of a predetermined dispensation schedule or per manually selected bolus amounts. Multiple delivery modes by an implantable device are described in US20210236731A1 which is incorporated with this specification in its entirety.

In an embodiment, the implantable device herein can utilize a closed-loop feedback control system to ensure accurate drug delivery. The implantable device may include a closed loop unit that includes the one or more sensors (and information/output sensed by them) and delivery of the one or more drugs and is guided by the artificial intelligence system for feedback control as described in US20140088554A1 which is incorporated with this specification in its entirety. This allows output from the one or more sensors to be used to directly modulate or control the rate of drug delivery from the drug delivery orifice, including stopping drug delivery altogether. The system is adapted to perform one or more of the following: collect the data from the one or more sensors, combine it with other diagnostic data that may have been gathered by a caregiver, perform a deep analysis of the disease state of the patient, arrive at a diagnosis, and decide whether to alter the drug delivery rate based on this diagnosis. The results of the computerized analysis of the disease state and the subsequent diagnosis can be reviewed by the caregiver and altered, modified, or rejected altogether. In these feedback loops, the caregiver can affect and control the operation of the system either through direct intervention at the point when the trigger to alter the state of the device is about to be reset, or at the point where the analysis of the disease state has been made by one or more computer executable methods that preferably utilize artificial intelligence to complete the analysis.

### 4. Use of AT for Intelligent Drug Dosing Based on Real Time Data

In an embodiment, the implantable device may determine the current health condition of the patient using a sensor. The implantable device may be used to provide dosage based on the health condition of the patient (e.g., human, animal, etc.). For example, consider the patient who is suffering from a disease. The implantable device may determine that the patient is affected by the disease using the sensor affixed to the body. The implantable device may also get notified about disease via an external device communicatively coupled to the implantable device.

The implantable device, then using artificial intelligence, extracts information regarding medication, dosage, and scheduled timings for medication from external medical database. In one embodiment, the external device may also provide the above extracted details as a command to the implantable device. In another embodiment, the implantable device may also extract prescriptions from an external medical database. The implantable device then may provide medication to the patient as per the scheduled timings at prescribed dosages. In one embodiment, the implantable device may also determine the current health condition of the patient and dynamically alter the medication in response to the current health condition. The implantable device may also be used to treat diseases like smallpox, dog bite, rabies, jaundice, viral fevers, pandemic diseases, etc.

### BB. Cybersecurity:

The IMD plays essential role in healthcare environment. The implantable medical device comprises a tiny computing platform with firmware. The implantable medical device may run on small batteries. The implantable medical device is programmable either internally, externally, or remotely. The implantable medical device is implanted within the body. The implantable medical device is capable of monitoring health status. The implantable medical device is further capable of delivering medical therapy. The implantable medical devices may be wireless implantable medical devices. Wireless access promotes usability and utility; and poses significant security and privacy concerns.

In one embodiment, the implantable medical device holds various data types such as static data, semi-static data, and dynamic data. The static data comprises device make information and model information (e.g., model number, serial number, etc.). The semi-static data comprises physician and health centre identification, patient name, and date of birth (DOB), medical condition, and therapy configuration. The dynamic data comprises patient health status history, therapy and dosage history, and audit logs.

The implantable medical device can be accessed/communicates in the following ways. The programmer device may communicate with the implantable medical device through wireless channels. The programmer device may communicate with the implantable medical device using radio frequency transmission. The personal computer communicates with the implantable medical device through USB-port “dongles” using radio frequencies. The PC may also be connected to Internet. The implantable medical device can be accessed remotely for one of the following scenarios: read data on health status & therapy history; emergency extraction of patient health history; emergency reset of IMD configuration; therapy programming/reprogramming; firmware updates; etc.

### 1. Regulation of Implantable Medical Devices:

In US, IMDs are regulated by Food and Drug Administration (FDA) Centre for Devices and Radiological Health (CDRH). The testing focus may be performed on the implantable medical device for safe and effective functioning, and different environmental conditions. The implantable medical device may be adapted to provide resistance/resilience to cyber-attacks (absence of focus). Are existing implantable device vulnerable?

The exiting devices are engineered without considering threat of a potential hacker. The current methods to prevent unauthorized access to IMDs include use of proprietary protocols; controlled access to “programmers” devices; essentially, security by obscurity!

The threats to the implantable medical device comprise patient data extraction; patient data tampering; device re-programming; repeated access attempts; device shut-off; therapy update; malicious inputs; data flooding. The vulnerability to the implantable medical device (IMD) comprises unsecured communication channels; inadequate authentication mechanisms; inadequate access controls; software vulnerabilities; weak audit mechanisms; meagre storage; insufficient alerts, etc. The risks associated with the implantable medical device (IMD) comprises patient health safety; patient privacy loss; inappropriate medical follow up; and device unavailability. The patient health safety may fail due to firmware malfunction; malicious therapy update; and malicious inputs to device. The patient privacy loss may occur due to data leakage from device. The inappropriate medical follow up may occur due to tampering of patient readings. The device unavailability may be due to battery power depletion and device flooding.

### 2. Risk-Based Mitigation Approach:

The above drawbacks can be overcome by risk-based mitigation approach. The risk-based mitigation approach is utilized to determine security impact for data types; implement adequate security mechanisms; and balance security/privacy with safety/usability. The risk-based mitigation approach comprises developing IMD security impact matrix; developing IMD access requirements matrix; selecting appropriate security mechanisms; and tailoring security mechanisms. The tailoring of security mechanisms comprises accommodating IMD environment constraints; adding compensating mechanisms (as needed).

The development of IMD security impact matrix comprises the following steps: identifying IMD data types (e.g., firmware, device identification, patient identification, provider identification, health condition, therapy configuration, patient readings, audit logs, etc.); identifying health delivering commands (e.g., emergency reset); analyzing impact of compromise i.e., for each data type, estimate impact (loss of confidentiality, integrity and availability) and for each command type, estimate impact (loss of availability); and assign impact as [LOW, MODERATE, HIGH]. The system tabulates the above details in IMD Security Impact Matrix as follows.

| | Emergency | | | Patient |
|---|---|---|---|---|
| Security Function/ | Reset | Patient | Therapy | Health |
| Data, Command | Command | ID Data | Data | Data |
| Confidentiality | N/A | MOD | LOW | MOD |
| Integrity | N/A | MOD | HIGH | HIGH |
| Availability | HIGH | LOW | MOD | MOD |

The determination of IMD access requirements comprises the following steps: developing matrix; adding required access privileges; and tabulating the details as IMD Access Requirements Matrix (IMD-ARM). The matrix development is performed by data type and health delivery command; by role of individual accessing IMD; and by access channels (e.g., wired, wireless). The required access privileges is added as per basic IMD functionality. The required access privileges is added by need for emergency access and by utility and quality of life factors. The system tabulates the above details in IMD access requirement Matrix as follows.

| | | | | Patient |
|---|---|---|---|---|
| ROLE-CHANNEL/ | Emergency | Patient | Therapy | Heath |
| Command, Data | Reset Cmd | ID Data | Data | Data |
| Patient-Wireless | | | | |
| Prescribing | | Read | Read | Read |
| Physician- | | Write | Write | |
| Wired | | | | |
| Maintenance | | Read | Read | Read |
| Physician- | | | | |
| Wireless | | | | |
| Emergency | Invoke | | | |
| Tech-Wireless | | | | |

The approach of selecting needed security mechanisms comprises overlaying IMD-IAM and IMD-ARM. The security mechanisms can be selected to protect IMD Data/Commands. The system selects the channel protection mechanisms from crypto protected channel and proprietary protocols. The system selects the authentication mechanisms as at least one of password; device-to-device handshake; cryptographic authentication. The system selects the audit mechanisms as at least one of auditable events and management of audit space depletion. The system selects the alert/alarm mechanisms as at least one of audible alarms and automatic device reset to safe mode.

The approach of tailoring security mechanism may be based on constraints that the IMD is subjected to. The IMD is subjected to constraints such as device size; cost; power; computational capability; and storage. The system adjusts the security mechanisms to accommodate the constraints. For example, the system adds alarm if authentication can't be strengthened for certain data types.

There are special challenges in securing the IMD such as battery and power limitations; use of cryptographic techniques; and audit mechanisms. The challenges with respect to battery and power limitation include minimization of power usage to extend battery life. Further battery depletion has devastating health consequences which has to take in account. The challenges with respect to use of cryptographic techniques includes highly constrained environment (cost, power, storage); compatible crypto suites/protocols needed (e.g., crypto for sensor networks). The challenges with respect to audit mechanisms comprises limited storage area on device; attacks may generate deluge of audit entries; managing audit space depletion; selective overwriting; and alarms (audible or to remote monitor).

In an embodiment, the system may incorporate one or more cybersecurity techniques. For example, the system may transmit data via a wired or wireless communications network, such as an NFC or cellular network. For instance, NFC can be used for communication with the implantable device and cellular network can be used for communication with the external device (e.g., wearable, smart phone, etc.). The communications network can provide access to the Internet.

In some embodiments, the system may implement various cybersecurity measures. For example, known credentials, PKI, encryption, and other cybersecurity operations may be applied between the implantable device and the external device to determine whether the external device should be allowed to conduct communications with the implantable device. Encryption key data may be exchanged between devices for future communications. Other device identifiers or data may be stored to control future communications. Upon establishment of a trusted relationship between the implantable device and the external device, the external device may be used to conduct remote programming session. The remote programming sessions may also be subjected to cybersecurity methods such as the use of credentials, PKI, encryption, etc.

In an embodiment, the system may incorporate one or more cybersecurity techniques. For example, the system may transmit data via a wired or wireless communications network, such as a Near Field Communication (NFC) or cellular network. For instance, NFC can be used for communication with the implantable device and cellular network can be used for communication with the external device (e.g., wearable, smart phone, etc.). The communications network can provide access to the Internet.

In some embodiments, the system may implement various cybersecurity measures. For example, known credentials, Public key infrastructure (PKI), encryption, and other cybersecurity operations may be applied between the implantable device and the external device to determine whether the external device should be allowed to conduct communications with the implantable device. Encryption key data may be exchanged between devices for future communications. Other device identifiers or data may be stored to control future communications. Upon establishment of a trusted relationship between the implantable device and the external device, the external device may be used to conduct remote programming sessions. The remote programming sessions may also be subjected to cybersecurity methods such as the use of credentials, PKI, encryption, etc.

In an embodiment, the system may comprise a cyber security module.

In an embodiment, the cyber security module further comprises an information security management module providing isolation between the system and the server.

In one aspect, a secure communication management (SCM) computer device for providing secure data connections is provided. The SCM computer device includes a processor in communication with memory. The processor is programmed to receive, from a first device, a first data message. The first data message is in a standardized data format. The processor is also programmed to analyze the first data message for potential cyber security threats. If the determination is that the first data message does not contain a cyber security threat, the processor is further programmed to convert the first data message into a first data format associated with the implantable device capability/environment and transmit the converted first data message to the system using a first communication protocol associated with the system comprising implantable device.

According to an embodiment, secure authentication for data transmissions comprises provisioning a hardware-based security engine (HSE) located in communications system, said HSE having been manufactured in a secure environment and certified in said secure environment as part of an approved network; performing asynchronous authentication, validation and encryption of data using said HSE, storing user permissions data and connection status data in an access control list used to define allowable data communications paths of said approved network, enabling communications of the communications system with other computing system subjects to said access control list, performing asynchronous validation and encryption of data using security engine including identifying a user device (UD) that incorporates credentials embodied in hardware using a hardware-based module provisioned with one or more security aspects for securing the system, wherein security aspects comprising said hardware-based module communicating with a user of said user device and said HSE.

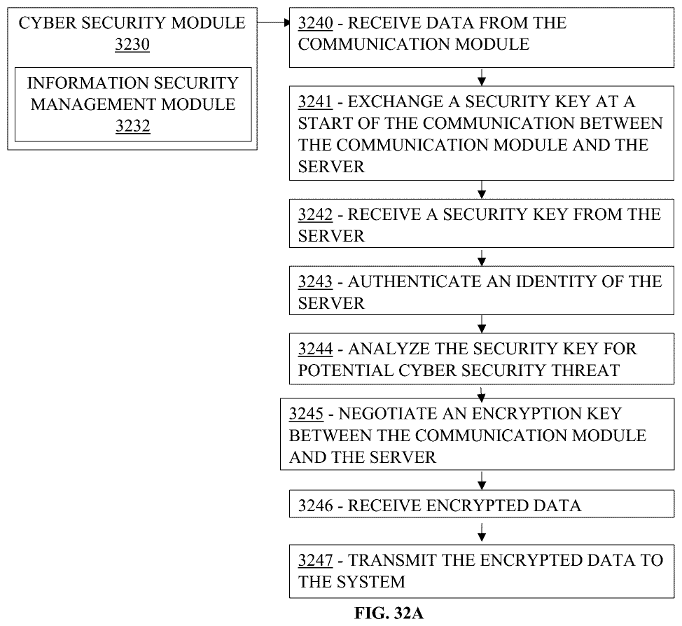

In an embodiment, FIG. 31 shows the block diagram of the cyber security module. The communication of data between the system 3100 and the server 3170 through the communication module 3112 is first verified by the information security management module 3132 before being transmitted from the system to the server or from the server to the system. The information security management module is operable to analyze the data for potential cyber security threats, to encrypt the data when no cyber security threat is detected, and to transmit the data encrypted to the system or the server.

### 3. Securing the Data Through the Cyber Security Module

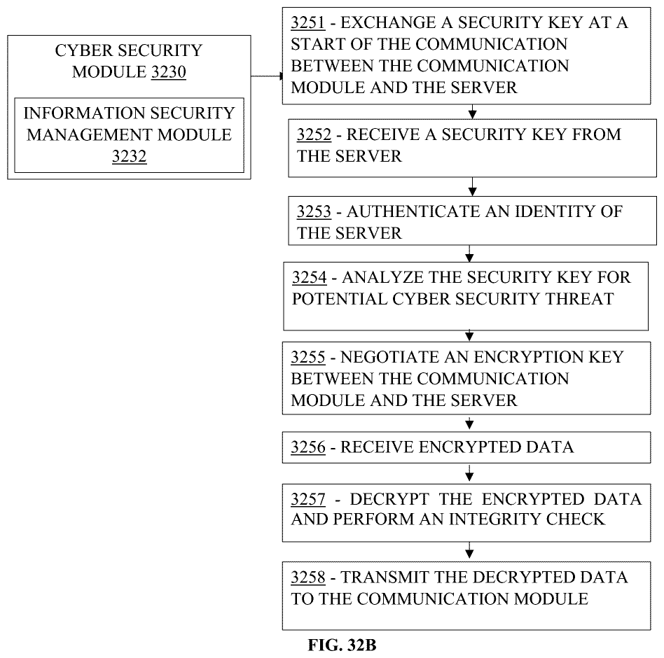

In an embodiment, the cyber security module comprises an information security management module providing isolation between the system and the server. FIG. 32A shows the flowchart of securing the data through the cyber security module 3230. At step 3240, the information security management module is operable to receive data from the communication module. At step 3241, the information security management module exchanges a security key at a start of the communication between the communication module and the server. At step 3242, the information security management module receives a security key from the server. At step 3243, the information security management module authenticates an identity of the server by verifying the security key. At step 3244, the information security management module analyzes the security key for potential cyber security threats. At step 3245, the information security management module negotiates an encryption key between the communication module and the server. At step 3246, the information security management module receives the encrypted data. At step 3247, the information security management module transmits the encrypted data to the server when no cyber security threat is detected.

In an embodiment, FIG. 32B shows the flowchart of securing the data through the cyber security module 3230. At step 3251, the information security management module is operable to: exchange a security key at a start of the communication between the communication module and the server. At step 3252, the information security management module receives a security key from the server. At step 3253, the information security management module authenticates an identity of the server by verifying the security key. At step 3254, the information security management module analyzes the security key for potential cyber security threats. At step 3255, the information security management module negotiates an encryption key between the communication module and the server. At step 3256, the information security management module receives encrypted data. At step 3257, the information security management module decrypts the encrypted data, and performs an integrity check of the decrypted data. At step 3258, the information security management module transmits the decrypted data to the communication module when no cyber security threat is detected.

In an embodiment, the integrity check is a hash-signature verification using a Secure Hash Algorithm 256 (SHA256) or a similar method.

In an embodiment, the information security management module is configured to perform asynchronous authentication and validation of the communication between the communication module and the server.

In an embodiment, the information security management module is configured to raise an alarm if a cyber security threat is detected. In an embodiment, the information security management module is configured to discard the encrypted data received if the integrity check of the encrypted data fails.

In an embodiment, the information security management module is configured to check the integrity of the decrypted data by checking accuracy, consistency, and any possible data loss during the communication through the communication module.

In an embodiment, the server is physically isolated from the system through the information security management module. When the system communicates with the server as shown in FIG. 31, identity authentication is first carried out on the system and the server. The system is responsible for communicating/exchanging a public key of the system and a signature of the public key with the server. The public key of the system and the signature of the public key are sent to the information security management module. The information security management module decrypts the signature and verifies whether the decrypted public key is consistent with the received original public key or not. If the decrypted public key is verified, the identity authentication is passed. Similarly, the system and the server carry out identity authentication on the information security management module. After the identity authentication is passed on to the information security management module, the two communication parties, the system, and the server, negotiate an encryption key and an integrity check key for data communication of the two communication parties through the authenticated asymmetric key. A session ID number is transmitted in the identity authentication process, so that the key needs to be bound with the session ID number; when the system sends data to the outside, the information security gateway receives the data through the communication module, performs integrity authentication on the data, then encrypts the data through a negotiated secret key, and finally transmits the data to the server through the communication module. When the information security management module receives data through the communication module, the data is decrypted first, integrity verification is carried out on the data after decryption, and if verification is passed, the data is sent out through the communication module; otherwise, the data is discarded.

In an embodiment, the identity authentication is realized by adopting an asymmetric key with a signature.

In an embodiment, the signature is realized by a pair of asymmetric keys which are trusted by the information security management module and the system, wherein the private key is used for signing the identities of the two communication parties, and the public key is used for verifying that the identities of the two communication parties are signed. Signing identity comprises a public and a private key pair. In other words, signing identity is referred to as the common name of the certificates which are installed in the user's machine (e.g., clinician's device).

In an embodiment, both communication parties need to authenticate their own identities through a pair of asymmetric keys, and a task in charge of communication with the information security management module of the system is identified by a unique pair of asymmetric keys.

In an embodiment, the dynamic negotiation key is encrypted by adopting an Rivest-Shamir-Adleman (RSA) encryption algorithm. RSA is a public-key cryptosystem that is widely used for secure data transmission. The negotiated keys include a data encryption key and a data integrity check key.

In an embodiment, the data encryption method is a Triple Data Encryption Algorithm (3DES). The integrity check algorithm is a Hash-based Message Authentication Code (HMAC-MD5-128) algorithm. When data is output, the integrity check calculation is carried out on the data, the calculated Message Authentication Code (MAC) value is added with the header of the value data message, then the data (including the MAC of the header) is encrypted by using a 3DES algorithm, the header information of a security layer is added after the data is encrypted, and then the data is sent to the next layer for processing. In an embodiment the next layer refers to a transport layer in the Transmission Control Protocol/Internet Protocol (TCP/IP) model.

The information security management module ensures the safety, reliability, and confidentiality of the communication between the system and the server through the identity authentication when the communication between the two communication parties starts the data encryption and the data integrity authentication. The method is particularly suitable for an embedded platform which has less resources and is not connected with a Public Key Infrastructure (PKI) system and can ensure that the safety of the data on the server cannot be compromised by a hacker attack under the condition of the Internet by ensuring the safety and reliability of the communication between the system and the server.

### 4. Cybersecurity in Close Range Communication:

The wireless communication between the implantable device and the external devices are prone to cyber attacks. The close range communication tag (e.g., RFID tag) may be affixed to the implantable device and an RFID reader may be used to read the information stored in the RFID tag. The tag may be secured so that the tag do not affect the patient. The tag may also be secured in order to get sealed from the liquid/drug within the implantable device.

The communication between the implantable device and the database should be secured over the three communication pathways: the communication pathway between the tag and the reader and the communication pathway between the reader and the database over the Internet. The communication pathway between the tag and the reader may be secured using cryptography or randomized hash lock algorithm. The communication pathway between the tag and the reader may also be secured using a hash function whenever the tag is read/scanned by the reader.

In an embodiment, the near-field communication region is small (e.g., 1 meter around the transmitting device in one implementation) and moveable along with the patient/user. The near-field communication region thus provides a well-defined cybersecurity attack region that is easily manageable, visually inspectable, and moveable with the user to make cybersecurity attack difficult. While transmissions within the near-field communication normally carry a high expectation of secure transfer, in some implementations, a further layer of security can be provided prior to communicating “secure data” within the near-field communication to provide another layer of protection in the event an eavesdropper device came within the near-field communication bubble.

In another embodiment, the body-coupled communication is used to fight against cyber-attacks. The body-coupled communication is one in which the human body is used as a transmission medium and communication is only possible near the patient.

In another embodiment, the system uses a proximity-based access control technique that uses ultrasonic distance bounding and only grants access to devices that are in its proximity.

### 5. Cybersecurity Using Cryptography:

In some implementations, cybersecurity secrets (such as public or private keys, digital signatures, and certificates, etc.) can be exchanged between devices to authenticate a new device to be paired, and to establish an encrypted channel that is within the near-field communication bubble. For example, an exchange of cybersecurity secrets (or equivalent) could be required to provide another hurdle for a potential eavesdropper. An exchange of cybersecurity secrets can include things such as a security key exchange protocol that allows an encrypted channel to be established, and other non-encryption or keys related secrets such as certificates, digital signatures, etc. depending on the specific implementation of the cybersecurity protocol.

For instance, the transmitting device and receiving devices would be required to successfully complete a security key exchange protocol before the transmitting device encrypts and transmits the data at each communication session. All of the devices that are intended to be part of the network could share a pre-established shared public and/or private keys known only by those devices (that is used to encrypt data), then even if an eavesdropper device was within the near-field communication bubble, the eavesdropper device could not process any data that is intercepted because they lack knowledge of the pre-established shared keys. Thus, once the cybersecurity secrets are exchanged, secure data (e.g., patient biometrics) can be encrypted by one device and then transmitted to the other and decrypted at another device. Furthermore, once a secure communication channel is established, the two communicating devices (e.g., infusion device and phone) can then be separated further, inside or outside the near-field (NF) bubble, and exchange encryption data over the air securely. Two types of encryption might be used, symmetric-key cryptography, in which the transmitting device and receiving device share a secret key, and public-key cryptography, in which two keys are used, one is public and the second is a secret.

In one embodiment, the implantable device is configured to monitor and control incoming and outgoing communication data with regard to the bidirectional communication connection. This means that the implantable device provides a cyber-secure “firewall” between the implantable device and its external connectivity system(s), allowing processes typically done only in-office to be done effectively and safely in a remote setting, where the patient has no other means to communicate with clinical staff than via a smartphone (i.e., on-demand device interrogation, on-demand device reprogramming).

In an embodiment, any client software running on PC, tablet, phone, etc., is essentially inherently prone to reverse engineering, cloning, and tampering. This means that software accessed by the user cannot be the end-all of cybersecurity tasks (though it does include many mitigations to make tampering with the system significantly harder). Instead, critical authentication activities should be done on remote hardware—ideally the implant itself. However, in the case of the implant described above, the implant's considerably limited resources and lack of an internet connection can prevent everything from being processed on the implant. Instead, according to the present disclosure, each cybersecurity task can be carefully considered and placed at the lowest priority level possible as resources permit (described further below). In the system described herein, some cybersecurity mitigations can occur on the implant, some can occur on the energizer or charger, some can occur in the user or software, and some cybersecurity mitigations can occur in the cloud.

The cybersecurity module of the implantable device restricts the unwanted access as well as unauthorized access.

In one embodiment, one or more steps in the sequence of establishing the first, second or third communication connections may require a “handshake” process that may: 1.) ensure cybersecurity and 2.) provide automatic data persistence, communication persistence and communication repetition to ensure that the communication between each component is completed and secured.

Network security is, in many embodiments, an important factor, and may impact a priority of transmission. Communications with highly sensitive information may be prioritized highly when network security is evaluated as being high, such as when highly secure devices are introduced to the system to create particularly secure communication links. Communications with relatively low sensitivity may be prioritized at a low level when security is high. Conversely, when highly secure devices are not available to the system, low-sensitivity information may be prioritized highly while high-sensitivity information may be prioritized at a low level.

6. Cybersecurity in Communication with External Devices:

Communications to and from hubs may take a variety of different forms. Common formats such as electronic mail, electronic text, audio, and video may be utilized in manners known in the art. Within such formats, particular messages may be included. In an embodiment, communications include alert messages. Such alert messages may be rated as highly importance. Such alert messages may be designed for particular destination nodes. In an embodiment, an alert message may be formatted for reception by a destination node associated with a medical professional, such as a clinic or hospital. In an embodiment, an alert message may be formatted for reception by a destination node associated with a caregiver of a patient, such as a home or office of an acquaintance or relative of the patient. The alert messages may contain information pertinent to the recipient, such as the identity of the sender, the location of the sender and relevant information which caused the alert to be sent.

The system is configured so that communication links and hubs are configured to provide communications back to a medical device from a destination node. In particular, the destination node may transmit a confirmation message to acknowledge receipt of a transmission. In various embodiments, destination nodes or a device, within a system may be utilized to transmit programming instructions to medical devices to reprogram medical devices or to command the transmission of more or different data. In particular, such a programming instruction or command may include stopping a false alert message, stopping an improper therapy delivery and configuring medical device into a safe operation mode to protect, at least in part, medical device from an environmental condition, such as a powerful electromagnetic field or abnormal temperatures.

As an addition and alternative to the security systems noted above, a proprietary data exchange format/interface that is kept secret may be used in communications between the implantable device and other devices (e.g., wearables, smart phone, etc.). However, even with secure dedicated lines or a secret data format, digital signatures will preferably be used to detect corruption of data. Additional implementations of security systems may also be utilized in accordance with the subject invention, including token-based and biometric security apparatus and methods to detect inalterable physical characteristics of persons attempting to access the data (e.g., dosage, patient health information, drug data, dosage schedule data, etc.) in order to authenticate the would-be user of the system and to ensure that the instrument being queried or otherwise used is proper. This is particularly important in distributed systems and systems permitting access to multiple implantable devices, or other components, or systems having multiple categories of users with different authorizations. Moreover, such systems as contemplated herein afford classification of users into categories where specialists would be allowed to use the validated instruments to interrogate and program the implantable devices, whereas non-specialists would only be able to interrogate such implantable devices or related system components or data bases. This use of IMD and instrument-related authentication facilitates systemic efficiencies and patient compliance in manners not previously experienced or fully contemplated. Indeed, the user interface is also enhanced by implementation of an option-rich authentication system. For example, one embodiment of the identification would be to automatically adjust system parameters to an authorized user's preference for screen displays, data emphasis, filtering, taskings, workflow schemes, feedback or reporting algorithms, and the like. Measures such as the foregoing may be used to authenticate the implantable devices and the network interfaces of the implantable devices, as well as other system architecture or components, and for persons accessing patient data, including a host patient, field personnel, technical service representatives, data harvesters, data managers, medical providers, and other authorized users. Other examples of use include user authentication of a programmer, extender, or other sensitive or controlled aspects of a medical device data system, including reports and system configurations.

In addition to the above security implementations, a preferred embodiment of the subject invention incorporates firewalls and/or proxy server technology. Such security measures not only protect patient data stored in data storage elements from access by unauthorized persons, but also protect the implantable devices and the network interfaces of the implantable devices from improper snooping and/or improper instruction from negligent or unscrupulous persons that may have access to the data network.

### 7. Biometric Access:

The system may use Biometrics to access the Implantable device. The biometrics may include voice, gesture, fingerprint, iris, etc. The system may use the dual level access control technique, in which first level uses patient biometrics and the second level uses the specific biometric verification (e.g., iris verification, fingerprint, etc.). In an embodiment, the caretaker may use the patients biometrics to access the data, which does not require much attention/effort from the patient. In another embodiment, the biometrics may be verified and authenticated when they are in proximity range. In yet another embodiment, the biometrics may not need to be in direct line of sight with the biometric reader for verification and authentication.

### 8. Battery Constraint Mitigation:

The cybersecurity attack may include battery draining. The battery draining attacks pose a serious problem to the implantable device, since battery replacement cannot be done usually without surgery. Other than reducing battery consumption by using lightweight encryption and external devices, other approaches can be used, such as relaxing the battery constrains by making the implant device wirelessly rechargeable, or harnessing kinetic energy from the human body. The implantable device may also recharge wirelessly using heat energy from the body. The security measures are discussed below to prevent the battery draining attack.

First, power-saving devices may be used so that the wake state can be extended by a data frame, and they periodically switch between wake mode and doze mode to perform power saving. These devices are used so that the wake mode is maintained only by a specific component (e.g., beacon frame). Direct battery draining attacks are difficult when the power saving devices are installed. Cyber-attacks like (battery draining attacks) are difficult to perform because they must be performed through the AP (access point), or rogue AP attacks must be performed.

Second, the system extends the wake mode only when a shared secret key in the received frame is checked and found matched. The system does not extend the wake mode when a frame arrives or a certain field value has been set. If the shared secret key does not match, the system changes from power-saving mode to sleep mode. If Cyber-attacks like (battery draining attacks) are frequent, the implantable device enters a deep sleep and does not respond to triggering signals for a long time.

### CC. Device Failure Mitigation

A partial list of potential implant failure modes is listed by system and subsystem in Table 1A to Table 1I provided below. Various embodiments may require more or fewer failure modes depending on the chosen components and subsystems of the embodiment.

TABLE 1A
SYSTEM: BATTERY

| | IMMEDIATE | | | |
|---|---|---|---|---|
| | OPERATIONAL | | PROCESSOR | PROCESSOR |
| FAILURE | IMPACT | RISK | INDICATION | RESPONSE |
| Battery voltage | None | Potential | Battery Voltage | Report. Continue |
| lower than | | premature battery | sensor shows | operations until |
| expected | | failure or | lower battery | external response |
| | | unknown power | voltage than | |
| | | draw | expected | |
| Battery voltage | None | Potential | Battery Voltage | Report, continue |
| higher than | | premature battery | sensor shows | operations until |
| expected | | failure | higher battery | external response |
| | | | voltage than | |
| | | | expected | |
| Voltage dip | None | Potential | Battery Voltage | Report and |
| during | | premature battery | Sensor readings | follow test in, |
| communication | | failure or | | report again. |
| greater than | | communications | | Continue until |
| expected | | circuit faulty | | external |
| | | | | response. |
| Voltage dip | None | Potential | Battery Voltage | Report and |
| during valve/ | | premature battery | Sensor readings | follow test in, |
| shuttle/ pump | | failure or valve/ | | report again. |
| operation | | shuttle/ pump | | Continue until |
| greater than | | circuit faulty | | external |
| expected | | | | response. |
| Voltage dip | None | Possible | Battery Voltage | Report. External |
| during a | | communications | Sensor readings | resources may |
| successful | | circuit problem. | | measure signal |
| communication | | Check for | | strength and |
| less than | | successful | | provide direction |
| expected | | communications | | (shutdown, continue |
| | | and signal | | operations, etc.) |
| | | strength. | | |
| Voltage dip | None | Possible valve/ | Battery Voltage | Report, check for |
| during a | | shuttle /pump | Sensor readings | proper valve/shuttle/ |
| successful | | operation | | pump operation |
| valve/ shuttle/ | | problem. Check | | (next dose), Report |
| Pump operation | | for proper | | again. Continue |
| less than | | valve/shuttle/ | | operations until |
| expected | | pump operation | | external response. |
| Battery charge | None | Potential battery | Battery voltage | Report, continue |
| profile out of | | or charging | sensor and | operations until |
| range (temp, | | circuit failure | Battery charge | external response |
| voltage, current, | | | circuit | |
| etc.) | | | indicators | |

TABLE 1B
SYSTEM: VALVE/VALVE SEALS

| | | | | | |
|---|---|---|---|---|---|
| | IMMEDIATE | | | | |
| | OPERATIONAL | | PROCESSOR- | PROCESSOR | DESIGN RISK- |
| FAILURE | IMPACT | RISK | INDICATION | RESPONSE | REDUCTION |

Sub-system: Valve (mechanical)

| | | | | | |
|---|---|---|---|---|---|
| Valve stuck in | Critical Risk | Unintended | Piston Position | Report | Flow |
| open position | | dosing | Sensor readings | until | restrictor |
| | | | | response. | |
| | | | | Start | |
| | | | | continuous | |
| | | | | attempt to | |
| | | | | close valve | |
| Valve closes | Unit is Non- | Cessation of | Piston Position | Report, | Flow |
| slower/later | Operational | dosing | Sensor readings | Cease | restrictor |
| than expected | | | | dosing. | |
| | | | | Wait for | |
| | | | | external | |
| | | | | response. | |
| Valve sticks | Critical Risk | Unintended | Piston Position | Report | Flow |
| partially open | | dosing | Sensor readings | until | restrictor |
| | | | | response. | |
| | | | | Start | |
| | | | | continuous | |
| | | | | attempt to | |
| | | | | close valve | |
| Valve stuck in | Implant is | Cessation of | Piston Position | Report, | |
| closed position | Non- | dosing | Sensor readings | Cease | |
| | Operational | | | dosing. | |
| | | | | Wait for | |
| | | | | external | |
| | | | | response. | |

Sub-system: Valve (electrical)

| | | | | |
|---|---|---|---|---|
| Actuator shorted | Implant is | Cessation of | Piston Position | Report |
| (includes | Non- | dosing | Sensor (no | |
| intermittent) | Operational | | movement), Battery | |
| | | | voltage Sensor | |
| | | | (significant voltage | |
| | | | dip) | |
| Actuator open | Implant is | Cessation of | Piston Position | Report |
| circuit (includes | Non- | dosing | Sensor (no | |
| intermittent) | Operational | | movement), Battery | |
| | | | voltage Sensor (No | |
| | | | voltage dip) | |

Sub-system: Valve (seals)

| | | | | |
|---|---|---|---|---|
| Seal leakage - | Critical Risk | Unintended | Piston Position | Report until |
| valve input | | dosing | Sensor | response. |
| to output | | | | Start |
| | | | | continuous |
| | | | | attempt to |
| | | | | close valve |
| Seal leakage - | None | Minimal or | None | None |
| Valve output to | | no leakage | | |
| electronics | | possible as | | |
| chamber | | battery, | | |
| | | PCB and | | |
| | | valve body | | |
| | | are potted | | |
| | | within tube. | | |
| Seal leakage - | None | Minimal or | None | None |
| Valve input to | | no leakage | | |
| electronics | | possible as | | |
| chamber | | battery, | | |
| | | PCB and | | |
| | | valve body | | |
| | | are potted | | |
| | | within tube. | | |

Sub-system: Valve Electronics (Processor)

| | | | | | |
|---|---|---|---|---|---|
| Processor failure | Non- | Cessation of | None | Lost | |
| (dead) while | Operational | dosing | | communications | |
| valve closed | | | | indication at | |
| | | | | Hub. Immediate | |
| | | | | valve closure | |
| | | | | command and | |
| | | | | confirmation | |
| | | | | from Piston | |
| | | | | Position | |
| | | | | Sensor. | |
| Processor failure | Non- | Cessation of | None | Lost | |
| (dead) while | Operational | dosing | | communications | |
| valve open (non | | (valve | | indication at | |
| latching valve) | | closes with | | Hub. Immediate | |
| | | loss of | | valve closure | |
| | | processor | | command and | |
| | | signal) | | confirmation | |
| | | | | from Piston | |
| | | | | Position | |
| | | | | Sensor. | |
| Processor failure | Critical Risk | Unintended | None | Lost | |
| (dead) while valve | | dosing | | communications | |
| open (latching | | | | indication at | |
| valve) | | | | Hub. Immediate | |
| | | | | valve closure | |
| | | | | command and | |
| | | | | confirmation | |
| | | | | from Piston | |
| | | | | Position | |
| | | | | Sensor. | |
| Processor reset (from | None | Repeat of | Reset exception | Report, pull | external clock |
| external influences | | error | | current time / | or NVRAM |
| e.g. MRI, CT scan, | | | | settings values | storage of |
| etc.) | | | | from NVRAM, | reset time / |
| | | | | continue | settings |
| | | | | operation | restores |
| | | | | until external | current |
| | | | | response. | settings |
| Watchdog timer reset | None | Repeat of | NVRAM | Report, pull | external clock |
| | | error | | current time / | or NVRAM |
| | | | | settings values | storage of |
| | | | | from NVRAM, | reset time / |
| | | | | continue | settings |
| | | | | operation | restores |
| | | | | until external | current |
| | | | | response. | settings |
| Processor fault | None | Repeat of | Processor fault | Report, pull | external clock |
| exception (code | | error | exception detected | current time / | or NVRAM |
| error or external | | | | settings values | storage of |
| influences) | | | | from NVRAM, | reset time / |
| | | | | continue | settings |
| | | | | operation | restores |
| | | | | until external | current |
| | | | | response. | settings |

Sub-system: Valve Electronics (Valve Drivers)

| | | | | | |
|---|---|---|---|---|---|
| Driver circuit | Non- | Cessation of | | Report, Cease | |
| failure causes | Operational | dosing | | dosing. Wait | |
| valve closure | | | | for external | |
| (non-latching | | | | response. | |
| valve) | | | | | |
| Driver circuit | Non- | Cessation of | | Report, Cease | |
| failure such that | Operational | dosing | | dosing. Wait | |
| valve is closed | | | | or external | |
| (latching valve) | | | | response. | |
| Driver circuit | Critical Risk | Cessation of | Piston Position | Report until | Time out |
| failure such that | (see Design | dosing | Sensor readings | response. Start | circuit that |
| valve remains open | Risk | | | continuous | gives valve |
| (latching valve) | Reduction) | | | attempt to | close command |
| | | | | close valve | after a certain |
| | | | | | period. |
| | | | | | Processor |
| | | | | | must regularly |
| | | | | | provide valve |
| | | | | | open command if |
| | | | | | valve is to |
| | | | | | stay open. |
| Driver circuit | Critical Risk | Unintended | Piston Position | Report until | Time out |
| failure such that | (see Design | dosing | Sensor and battery | response. Start | circuit that |
| valve is electrically | Risk | | Voltage readings | continuous | releases driver |
| held open (non- | Reduction) | | (constant drain) | attempt to | output. |
| latching valve) | | | | close valve | Processor |
| | | | | | must reset line |
| | | | | | if valve is to |
| | | | | | stay open. |

Sub-system: Valve Electronics (Communications)

| | | | | | |
|---|---|---|---|---|---|
| Communications | No | unable to | No | Continue | Expected |
| receiver failure | immediate | receive | commands/ | to report. | periodic |
| | risk | commands/ | queries | Continuation | communications |
| | | queries | received within | of dosing | |
| | | | expected period | | |
| No | No | No | No | Continue | Expected |
| Communications | immediate | commands/ | commands/ | to report. | periodic |
| received (No | risk | queries | queries | Continuation | communications |
| communications | | received | received within | of dosing | |
| from external | | within | expected period | | |
| devices within | | expected | | | |
| expected period) | | period | | | |
| Communications | No | unable to | Battery voltage | Continuation | External device |
| transmitter failure | immediate | send critical | (no drop on TX) | of dosing | responsible |
| | risk | reports | | | for action if |
| | | | | | no transmissions |
| | | | | | received |

Sub-system: Valve Electronics (Position sensor)

| | | | | |
|---|---|---|---|---|
| Faulty reading / | Non- | Cessation of | Position sensor does | Report, Cease |
| unexpected value | Operational | dosing | not indicate | dosing. Wait |
| | | | expected value | for external |
| | | | | response. |
| Greater than maximum | Non- | Cessation of | Position sensor data | Report, Cease |
| value | Operational | dosing | | dosing. Wait |
| | | | | for external |
| | | | | response. |
| Less than minimum | Non- | Cessation of | Position sensor data | Report, Cease |
| value | Operational | dosing | | dosing. Wait |
| | | | | for external |
| | | | | response. |
| Maximum value (when | Non- | Cessation of | Position sensor data | Report, Cease |
| unexpected) | Operational | dosing | | dosing. Wait |
| | | | | for external |
| | | | | response. |
| Minimum value (when | Non- | Cessation of | Position sensor data | Report, Cease |
| unexpected) | Operational | dosing | | dosing. Wait |
| | | | | for external |
| | | | | response. |

TABLE 1C
SYSTEM: PISTON

| | | | | |
|---|---|---|---|---|
| | IMMEDIATE | | | |
| | OPERATIONAL | | PROCESSOR | PROCESSOR |
| FAILURE | IMPACT | RISK | INDICATION | RESPONSE |

Sub-system: Piston Seal

| | | | | |
|---|---|---|---|---|
| Piston seal | Non-Operational | Cessation | Piston Position | Report, Cease |
| stuck (piston | | of dosing | Sensor | dosing. Wait for |
| does not move | | | | external response. |
| when valve | | | | |
| open) | | | | |
| Piston Seal | Diluted dosing | Diluted | Piston Position | Report, Continue |
| leakage | | dose, | Sensor, Pressure | dosing (may be |
| | | reduction in | sensor readings | diluted). Wait for |
| | | osmotic | | external response. |
| | | pressure | | |

Sub-system: Piston Position

| | | | | |
|---|---|---|---|---|
| Piston | None | Potential | Piston Position | Report, Continue |
| movement | | for eventual | Sensor Readings | dosing. |
| less/slower than | | stuck Piston | | |
| expected | | seal | | |
| Piston | None | Minimal as | Piston Position | Report, Continue |
| movement | | pressure | Sensor readings | dosing unless |
| greater/faster | | will lessen | | increased piston |
| than expected | | per dose | | speed too high for |
| | | | | valve control |

TABLE 1D
SYSTEM: SHUTTLE / SHUTTLE VALVES

| | | | | |
|---|---|---|---|---|
| | | | | PROCESSOR |
| | IMMEDIATE | | | RESPONSE |
| | OPERATIONAL | | PROCESSOR | DESIGN RISK |
| FAILURE | IMPACT | RISK | INDICATION | REDUCTION |

Sub-system: Shuttle (mechanical)

| | | | | |
|---|---|---|---|---|
| Shuttle stuck | Unit is Non- | Cessation of | Piston Position | Report until |
| in position | Operational | dosing | Sensor readings, | response. Start |
| | | | Battery voltage | continuous |
| | | | readings | attempt to |
| | | | | unstick shuttle |
| Shuttle tines | Unit is Non- | Cessation of | Piston Position | Report until |
| slipping | Operational | dosing | Sensor readings | response. Start |
| | | | and Battery | continuous |
| | | | Voltage readings | attempt to |
| | | | | unstick shuttle |
| shuttle actuator | Unit is Non- | Cessation of | Piston Position | Report until |
| stuck | Operational | dosing | Sensor readings | response. Start |
| | | | and Battery | continuous |
| | | | Voltage readings | attempt to |
| | | | | unstick shuttle |
| shuttle actuator | None | Potential | Piston Position | Report, Continue |
| operation | | cessation of | Sensor readings | dosing. Wait for |
| intermittent | | dosing | | external |
| | | | | response. |

Sub system: Shuttle (electrical)

| | IMMEDIATE | | | |
|---|---|---|---|---|
| | OPERATIONAL | | PROCESSOR | PROCESSOR |
| FAILURE | IMPACT | RISK | INDICATION | RESPONSE |
| Actuator | Implant is Non- | Cessation of | Piston Position | Report, cessation |
| shorted | Operational | dosing | Sensor (no | of dosing |
| (includes | | | movement), | |
| intermittent) | | | Battery voltage | |
| | | | Sensor | |
| | | | (significant | |
| | | | voltage dip) | |
| Actuator open | Implant is Non- | Cessation of | Piston Position | Report, cessation |
| circuit | Operational | dosing | Sensor (no | of dosing |
| (includes | | | movement), | |
| intermittent) | | | Battery voltage | |
| | | | Sensor (No | |
| | | | voltage dip) | |

Sub system: Electronics - Processor

| | IMMEDIATE | | | | |
|---|---|---|---|---|---|
| | OPERATIONAL | | PROCESSOR | PROCESSOR | DESIGN RISK |
| FAILURE | IMPACT | RISK | INDICATION | RESPONSE | REDUCTION |
| Processor | Non- | Cessation of | None | Backup | |
| failure (dead) | Operational | dosing | | processor | |
| | | | | used if | |
| | | | | available | |
| Processor | None | Repeat of | Reset | Report, pull | external |
| reset (from | | error causing | exception | current time / | clock or |
| external | | reset | | settings | NVRAM |
| influences eg. | | | | values from | storage of |
| MRI, CT | | | | NVRAM, | reset time / |
| scan, etc.) | | | | continue | settings |
| | | | | operation | restores |
| | | | | until external | current |
| | | | | response. | settings |
| Watchdog | None | Repeat of | NVRAM | Report, pull | external |
| timer reset | | error | | current time / | clock or |
| | | | | settings | NVRAM |
| | | | | values from | storage of |
| | | | | NVRAM, | reset time / |
| | | | | continue | settings |
| | | | | operation | restores |
| | | | | until external | current |
| | | | | response. | settings |
| Processor | None | Repeat of | Processor | Report, pull | external |
| fault | | error | fault | current time / | clock or |
| exception | | | exception | settings | NVRAM |
| (code error or | | | detected | values from | storage of |
| external | | | | NVRAM, | reset time / |
| influences) | | | | continue | settings |
| | | | | operation | restores |
| | | | | until external | current |
| | | | | response. | settings |

Sub system: Electronics - shuttle driver

| | IMMEDIATE | | | |
|---|---|---|---|---|
| | OPERATIONAL | | PROCESSOR | PROCESSOR |
| FAILURE | IMPACT | RISK | INDICATION | RESPONSE |
| Driver circuit | Non- | Cessation of | | Report, Cease |
| failure - shuttle | Operational | dosing | | dosing. Attempt |
| not driven | | | | system restart |
| | | | | and retest. Wait |
| | | | | for external |
| | | | | response. |

Sub system: Electronics - Communications

| | IMMEDIATE | | | | |
|---|---|---|---|---|---|
| | OPERATIONAL | | PROCESSOR | PROCESSOR | DESIGN RISK |
| FAILURE | IMPACT | RISK | INDICATION | RESPONSE | REDUCTION |
| Communications | No | unable to | No | Continue to | Expected |
| receiver | immediate | receive | commands/ | report. | periodic |
| failure | risk | commands/ | queries | continuation | communications |
| | | queries | received | of dosing | |
| | | | within | | |
| | | | expected | | |
| | | | period | | |
| No | No | No | No | Continue to | Expected |
| Communications | immediate | commands/ | commands/ | report. | periodic |
| received | risk | queries | queries | continuation | communications |
| No | | received | received | of dosing | |
| communications | | within | within | | |
| from | | expected | expected | | |
| external | | period | period | | |
| devices | | | | | |
| within | | | | | |
| expected | | | | | |
| period) | | | | | |
| Communications | No | unable to | Battery | Continuation | External |
| transmitter | immediate | send critical | voltage (no | of dosing | device |
| failure | risk | reports | drop on TX) | | responsible |
| | | | | | for action if |
| | | | | | no |
| | | | | | transmissions |
| | | | | | received |

| | | | | |
|---|---|---|---|---|
| | IMMEDIATE | | | |
| | OPERATIONAL | | PROCESSOR | PROCESSOR |
| FAILURE | IMPACT | RISK | INDICATION | RESPONSE |

Sub system: Electronics - Position sensor

| | | | | |
|---|---|---|---|---|
| Faulty reading/ | Non-Operational | Cessation of | Position sensor | Report, Cease |
| unexpected | | dosing | does not | dosing. Wait for |
| value | | | indicated | external |
| | | | expected value | response. |
| Greater than | Non-Operational | Cessation of | Position sensor | Report, Cease |
| maximum | | dosing | data | dosing. Wait for |
| value | | | | external |
| | | | | response. |
| Less than | Non-Operational | Cessation of | Position sensor | Report, Cease |
| minimum | | dosing | data | dosing. Wait for |
| value | | | | external |
| | | | | response. |
| Maximum | Non-Operational | Cessation of | Position sensor | Report, Cease |
| value (when | | dosing | data | dosing. Wait for |
| unexpected) | | | | external |
| | | | | response. |
| Minimum | Non-Operational | Cessation of | Position sensor | Report, Cease |
| value (when | | dosing | data | dosing. Wait for |
| unexpected) | | | | external |
| | | | | response. |
| Faulty reading/ | Non-Operational | Cessation of | Position sensor | Report, Cease |
| unexpected | | dosing | does not | dosing. Wait for |
| value | | | indicated | external |
| | | | expected value | response. |

Sub system: Shuttle seals / Shuttle valves seals

| | | | | |
|---|---|---|---|---|
| Piston seal | Non-Operational | Cessation of | Piston Position | Report until |
| stuck (piston | | dosing | Sensor | response. Start |
| does not move | | | | continuous |
| when | | | | attempt to |
| operating | | | | unstick shuttle |
| shuttle) | | | | |
| Piston Seal | Diluted dosing | Diluted dose, | Piston Position | Report, Continue |
| leakage | | reduction in | Sensor, Pressure | dosing (may be |
| | | osmotic pressure | sensor readings | diluted). Wait |
| | | | | for external |
| | | | | response. |

Sub system: Shuttle position

| | | | | |
|---|---|---|---|---|
| Piston | None | Potential for | Piston Position | Report, Continue |
| movement less | | eventual stuck | Sensor Readings | dosing. |
| than expected | | Piston seal | | |
| Piston | None | Minimal as | Piston Position | Report, Continue |
| movement | | pressure will | Sensor readings | dosing unless |
| greater than | | lessen per dose | | increased piston |
| expected | | | | speed too high |
| | | | | for shuttle |
| | | | | control |

TABLE 1E
SYSTEM: PUMP / PUMP VALVES

| | | | | |
|---|---|---|---|---|
| | IMMEDIATE | | | |
| | OPERATIONAL | | PROCESSOR | PROCESSOR |
| FAILURE | IMPACT | RISK | INDICATION | RESPONSE |

Pump (mechanical)

| | | | | |
|---|---|---|---|---|
| Pump Valve | Unit is Non- | Cessation of | Piston | Report, Cease dosing. Wait |
| (input or | Operational | dosing | Position | for external response. |
| output) stuck | | | Sensor | |
| in open/ | | | readings, | |
| partially | | | Battery | |
| open | | | voltage | |
| position, | | | readings | |
| includes | | | | |
| valve seal | | | | |
| leakage | | | | |
| Pump Valve | Unit is Non- | Cessation of | Piston | Report until response. Start |
| (input or | Operational | dosing | Position | continuous attempt to |
| output) stuck | | | Sensor | unstick pump |
| in closed | | | readings and | |
| position | | | Battery | |
| | | | Voltage | |
| | | | readings | |
| Pump | Unit is Non- | Cessation of | Piston | Report until response. Start |
| actuator | Operational | dosing | Position | continuous attempt to |
| stuck | | | Sensor | unstick pump |
| | | | readings and | |
| | | | Battery | |
| | | | Voltage | |
| | | | readings | |
| Pump | None | Potential | Piston | Report, Continue dosing. |
| actuator | | cessation of | Position | Wait for external response. |
| operation | | dosing | Sensor | |
| intermittent | | | readings | |

Sub system: Pump (electrical)

| | | | | |
|---|---|---|---|---|
| Actuator | Implant is | Cessation of | Piston | Report, cessation of dosing |
| shorted | Non- | dosing | Position | |
| (includes | Operational | | Sensor (no | |
| intermittent) | | | movement), | |
| | | | Battery | |
| | | | voltage Sensor | |
| | | | (significant | |
| | | | voltage dip) | |
| Actuator | Implant is | Cessation of | Piston | Report, cessation of dosing |
| open circuit | Non- | dosing | Position | |
| (includes | Operational | | Sensor (no | |
| intermittent) | | | movement), | |
| | | | Battery | |
| | | | voltage Sensor | |
| | | | (No voltage | |
| | | | dip) | |

| | | | | | |
|---|---|---|---|---|---|
| | IMMEDIATE | | | | |
| | OPERATIONAL | | PROCESSOR | PROCESSOR | DESIGN RISK |
| FAILURE | IMPACT | RISK | INDICATION | RESPONSE | REDUCTION |

Sub system: Electronics - Processor

| | | | | | |
|---|---|---|---|---|---|
| Processor | Non- | Cessation of | None | Backup | |
| failure (dead) | Operational | dosing | | processor | |
| | | | | used if | |
| | | | | available | |
| Processor | None | Repeat of | Reset | Report, pull | external |
| reset (from | | error causing | exception | current time/ | clock or |
| external | | reset | | settings | NVRAM |
| influences | | | | values from | storage of |
| eg. MRI, CT | | | | NVRAM, | reset time / |
| scan, etc.) | | | | continue | settings |
| | | | | operation | restores |
| | | | | until | current |
| | | | | external | settings |
| | | | | response. | |
| Watchdog | None | Repeat of | NVRAM | Report, pull | external |
| timer reset | | error | | current time/ | clock or |
| | | | | settings | NVRAM |
| | | | | values from | storage of |
| | | | | NVRAM, | reset time / |
| | | | | continue | settings |
| | | | | operation | restores |
| | | | | until | current |
| | | | | external | settings |
| | | | | response. | |
| Processor | None | Repeat of | Processor fault | Report, pull | external |
| fault | | error | exception | current time/ | clock or |
| exception | | | detected | settings | NVRAM |
| (code error | | | | values from | storage of |
| or external | | | | NVRAM, | reset time / |
| influences) | | | | continue | settings |
| | | | | operation | restores |
| | | | | until | current |
| | | | | external | settings |
| | | | | response. | |

Sub system: Electronics - Pump driver

| | | | |
|---|---|---|---|
| Driver circuit | Non- | Cessation of | Report, |
| failure - | Operational | dosing | Cease |
| pump not | | | dosing. |
| driven | | | Attempt |
| | | | system |
| | | | restart and |
| | | | retest. Wait |
| | | | for external |
| | | | response. |

Sub system: Electronics - Communications

| | | | | | |
|---|---|---|---|---|---|
| Communications | No immediate | unable to | No | Continue to | Expected |
| receiver | risk | receive | commands/ | report. | periodic |
| failure | | commands/ | queries | continuation | communications |
| | | queries | received | of dosing | |
| | | | within | | |
| | | | expected | | |
| | | | period | | |
| No | No immediate | No | No | Continue to | Expected |
| Communications | risk | commands/ | commands/ | report. | periodic |
| received | | queries | queries | continuation | communications |
| (No | | received | received | of dosing | |
| communications | | within | within | | |
| from | | expected | expected | | |
| external | | period | period | | |
| devices | | | | | |
| within | | | | | |
| expected | | | | | |
| period) | | | | | |
| Communications | No immediate | unable to send | Battery | Continuation | External |
| transmitter | risk | critical reports | voltage (no | of dosing | device |
| failure | | | drop on TX) | | responsible |
| | | | | | for action if |
| | | | | | no transmissions |
| | | | | | received |

| | | | | |
|---|---|---|---|---|
| | IMMEDIATE | | | |
| | OPERATIONAL | | PROCESSOR | PROCESSOR |
| FAILURE | IMPACT | RISK | INDICATION | RESPONSE |

Sub system: Electronics - Position sensor

| | | | | |
|---|---|---|---|---|
| Faulty | Non- | Cessation of | Position | Report, Cease dosing. Wait |
| reading / | Operational | dosing | sensor does | for external response. |
| unexpected | | | not indicated | |
| value | | | expected value | |
| Greater than | Non- | Cessation of | Position | Report, Cease dosing. Wait |
| maximum | Operational | dosing | sensor data | for external response. |
| value | | | | |
| Less than | Non- | Cessation of | Position | Report, Cease dosing. Wait |
| minimum | Operational | dosing | sensor data | for external response. |
| value | | | | |
| Maximum | Non- | Cessation of | Position | Report, Cease dosing. Wait |
| value (when | Operational | dosing | sensor data | for external response. |
| unexpected) | | | | |
| Minimum | Non- | Cessation of | Position | Report, Cease dosing. Wait |
| value (when | Operational | dosing | sensor data | for external response. |
| unexpected) | | | | |

Sub system: Seal

| | | | | |
|---|---|---|---|---|
| Piston seal | Non- | Cessation of | Piston | Report, Cease dosing. Wait |
| stuck (piston | Operational | dosing | Position | for external response. |
| does not | | | Sensor | |
| move when | | | | |
| pumping) | | | | |
| Piston Seal | Diluted dosing | Diluted dose, | Piston | Report, Continue dosing |
| leakage | | reduction in | Position | (may be diluted). Wait for |
| | | osmotic | Sensor, | external response. |
| | | pressure | Pressure | |
| | | | sensor | |
| | | | readings | |

Sub system: Pump Position

| | | | | |
|---|---|---|---|---|
| Piston | None | Potential for | Piston | Report, Continue dosing. |
| movement | | eventual stuck | Position | |
| less/slower | | Piston seal | Sensor | |
| than | | | Readings | |
| expected | | | | |
| Piston | None | Minimal as | Piston | Report, Continue dosing |
| movement | | pressure will | Position | unless increased piston |
| greater/ | | lessen per | Sensor | speed too high for pump |
| faster | | dose | readings | control |
| than | | | | |
| expected | | | | |

TABLE 1F
SYSTEM: DEVICE COMPONENT EXPOSED TO SUBJECT'S BODY

| | | | | | |
|---|---|---|---|---|---|
| | IMMEDIATE | | | | |
| | OPERATIONAL | | PROCESSOR | PROCESSOR | DESIGN RISK |
| FAILURE | IMPACT | RISK | INDICATION | RESPONSE | REDUCTION |

Sub system: Tube

| | | | | | |
|---|---|---|---|---|---|
| Tube vents | Non-Operational | Potential | Piston | Report, | Methods as |
| blocked | (See Design | cessation | Position | Continue | described |
| (scabbing, | Risk Reduction) | of dosing | Sensor | dosing if | elsewhere in this |
| immune | | | Reading (no | possible | application. |
| response, | | | or slow | | |
| etc.) | | | movement) | | |

Sub system: Endcap (battery end)

| | | | | | |
|---|---|---|---|---|---|
| Leakage | None | Minimal | None | Continue | Battery/PCB/Pu mp |
| past endcap | | or no | | operations | Body are potted |
| of | | leakage | | | |
| Interstitial | | possible | | | |
| fluid into | | as | | | |
| battery, | | battery, | | | |
| PCB or pump | | PCB | | | |
| compartment | | and | | | |
| | | pump | | | |
| | | body are | | | |
| | | potted | | | |
| | | within | | | |
| | | tube | | | |

Sub system: Endcap (membrane end)

| | | | | |
|---|---|---|---|---|
| Leakage | None | Cessation | Piston | Report, |
| (osmotic | | of dosing | Position | Continue |
| fluid to | | (eventual | Sensor | dosing if |
| interstitial | | loss of | (slower or no | possible |
| fluid) | | osmotic | movement), | |
| | | pressure) | Pressure | |
| | | | sensor | |

TABLE 1G
SYSTEM: OSMOTIC UNIT

| | | | | | |
|---|---|---|---|---|---|
| | IMMEDIATE | | | | |
| | OPERATIONAL | | PROCESSOR | PROCESSOR | DESIGN RISK |
| FAILURE | IMPACT | RISK | INDICATION | RESPONSE | REDUCTION |

Sub system: Osmotic membrane support

| | | | | | |
|---|---|---|---|---|---|
| Leakage | None | Cessation | Piston Position | Report, | Strong |
| around | | of dosing | Sensor (slower | Continue | adhesive |
| membrane | | (eventual | or no | dosing if | should prevent |
| (osmotic | | loss of | movement), | possible | this. |
| fluid to | | osmotic | Pressure | | |
| interstitial | | pressure) | sensor | | |
| fluid) | | | | | |

Sub system: Osmotic membrane

| | | | | | |
|---|---|---|---|---|---|
| Membrane | Non-Operational | Cessation | Piston Position | Report, Cease | Design |
| blockage | (see Design Risk | of dosing | Sensor (no | dosing. Wait | enhancements |
| | Reduction) | | movement), | for external | and other |
| | | | Pressure | response. | methods as |
| | | | Sensor (no | | described |
| | | | pressure) | | elsewhere in |
| | | | | | this |
| | | | | | application. |
| Membrane | None | Potential | Piston Position | Report, | Design |
| partial | | cessation | Sensor (slower | Continue | enhancements |
| blockage | | of dosing | piston | dosing if | and other |
| | | | movement | possible | methods as |
| | | | than expected), | | described |
| | | | | | elsewhere in |
| | | | | | this |
| | | | | | application. |
| Membrane | Non-Operational | Cessation | Piston Position | Report, Cease | Ceramic |
| integrity | (see Design Risk | of dosing | Sensor (no | dosing. Wait | membrane for |
| failure | Reduction) | | movement), | for external | strength |
| (tear, hole, | | | Pressure | response. | |
| etc.) | | | Sensor (no | | |
| | | | pressure) | | |

TABLE 1H
SYSTEM: PHARMACEUTICAL

| | IMMEDIATE | | | | |
|---|---|---|---|---|---|
| | OPERATIONAL | | PROCESSOR | PROCESSOR | DESIGN RISK |
| FAILURE | IMPACT | RISK | INDICATION | RESPONSE | REDUCTION |
| Aggregation, | Critical Risk | Cessation/ | Piston | Report, | US FDA |
| settling, or | (see Design | reduction of | Position | Continue | approved |
| flocculation | Risk | dosing and/or | Sensor | dosing if | pharmaceuticals |
| (pharmaceutical | Reduction) | diluted dose | (slower | possible | for implantable |
| dependent) | | | or no | | dosing. |
| | | | movement) | | |

TABLE 1I
SYSTEM: EXTERNAL CONDITIONS

| | | | | | |
|---|---|---|---|---|---|
| | IMMEDIATE | | | | |
| | OPERATIONAL | | PROCESSOR | PROCESSOR | DESIGN RISK |
| FAILURE | IMPACT | RISK | INDICATION | RESPONSE | REDUCTION |

Sub system: Patient trauma

| | | | | | |
|---|---|---|---|---|---|
| Tube bent | Non- | Potential | Piston | Report, | |
| due to | Operational | cessation of | Position | Cease | |
| Patient | | dosing/ seal | Sensor (no | dosing. Wait | |
| traumatic | | or endcap | movement), | for external | |
| experience | | leakage. | Pressure | response. | |
| | | Pharmaceutical | Sensor | | |
| | | leak | (significant | | |
| | | | pressure | | |
| | | | change) | | |
| Tube | Critical Risk | Unintended | Piston | Report until | |
| leakage due | | dosing | Position | response. | |
| to Patient | | | Sensor (no | | |
| traumatic | | | movement), | | |
| experience | | | Pressure | | |
| | | | Sensor (no | | |
| | | | pressure) | | |
| Movement | None | Loss of | Loss of | Continual | |
| under skin | | communications | communications | attempt to | |
| | | due to | with | report. | |
| | | unknown | external | Continue | |
| | | location | devices | Operations. | |
| Fever | None | None. | Sensed | Report, | Pharmaceutical |
| | | Heightened | Temperature | Continue | Fluid designed |
| | | temperature | rise | operations | for fever grade |
| | | no threat to | | | temperatures |
| | | Pharmaceutical | | | |
| | | or device | | | |

Sub system: External influences

| | | | | | |
|---|---|---|---|---|---|
| MRI - | Potential Non- | Processor | Processor | Report, See | Remove or |
| During an | operational | failure, local | exceptions or | Processor | inactivate |
| MRI scan, the | | heating | other failures. | failure | implantable |
| radiofrequency | | | Temperature | response | device |
| field from | | | sensor(s) | above. | before MRI |
| the scanner's | | | increase | | |
| transmit | | | | | |
| coil, but also | | | | | |
| the switched | | | | | |
| gradient | | | | | |
| fields, induce | | | | | |
| currents in any | | | | | |
| conductive | | | | | |
| object in the | | | | | |
| bore. This | | | | | |
| makes any | | | | | |
| metallic | | | | | |
| medical | | | | | |
| implant an | | | | | |
| additional | | | | | |
| risk for an | | | | | |
| MRI patient, | | | | | |
| because those | | | | | |
| currents can | | | | | |
| heat up the | | | | | |
| surrounding | | | | | |
| tissues to | | | | | |
| dangerous | | | | | |
| levels. | | | | | |
| CT Scan - | Potential Non- | Potential | Processor | Report, See | Remove or |
| Significant | operational | Processor | exceptions or | Processor | inactivate |
| electrical | | errors | other failures. | failure | implantable |
| current | | | Temperature | response | device |
| (current | | | sensor(s) | above. | before X-ray |
| through | | | increase | | or CT scan. |
| body) could | | | | | |
| cause | | | | | |
| processor | | | | | |
| issues. | | | | | |

The IMMEDIATE OPERATIONAL IMPACT and RISK columns describe the severity of the failure and the risk associated with that failure. All the failure modes in the Table 1A to Table 1I generate a report. A report may cause an immediate data communication with external resources, or the report may be stored until the next external communication or until the device is removed from the Patient and data can be downloaded. Data transmitted or stored may comprise timestamp, requested dose size and period, actual dosing size, dosing periods, piston position, component temperatures, battery voltage, current draw, and piston position over time. This data may be collected before, during and after an action such as valve actuation, communications, and waking up or going into standby.

Table 1A to Table 1I generally assumes the implant has a piston position sensor and a method of measuring battery voltage. The implant may also comprise a pressure sensor located in the osmotic or pharmaceutical compartments; a real time clock or other method of maintaining relative time regardless of processor reset or exception; a method of communicating with a device or devices outside the body; a processor with watchdog timer capability; a method of recharging the implant's power source and appropriate methods of monitoring the recharging; and a temperature sensor on components such as the processor, valve, battery, and valve driver circuitry.

In some embodiments, electronics are potted/encased against moisture and valve coil(s) are potted. In some embodiments, minimal or no volume is available for fluid ingress. In some embodiments, various pulse patterns may be applied to unstick the valve. This pulse pattern may be structured to create an oscillation at the resonant frequency of the valve. This may be repeated up until battery life for communications are compromised. Once communications are established and failure information transferred, attempts to unstick the valve continue until battery life is over. In some embodiments, a flow restrictor at the valve prevents excessive flow in a valve stuck open scenario. Flow may be restricted to the point that a fully stuck open valve will only provide the maximum system dose. In some embodiments, an expected periodic communication (2 or 3 component system) provides an indication to the implanted device as well as external devices of a communications or systems failure. In some embodiments, the endcap Leakage may be due to damage sustained before insertion or from trauma to the Patient while the device is implanted. In some embodiments, the effects of a bent tube depend on the severity of the bend and range from no effect to cessation of dosing due to impeded piston travel and piston or valve seal leakage. In some embodiments, a likely faulty reading includes indications of reverse piston travel, constantly changing values (while valve closed), etc. The processor keeps track of piston position. If not within the range of possible values (speed, direction, total range, etc.), this is a potential indicator of a faulty position sensor. Other factors such as valve status will be taken into account. Upon this fault, the piston position will be recorded regularly for diagnostic purposes.

In some embodiments, the device or a system comprising the device is configured to test to see if next valve operation causes a higher-than-expected voltage dip. If yes, it is likely a faulty battery. If no, likely faulty communications circuit. Note: This test request may be generated externally or by the internal processor. In some embodiments, the device or a system comprising the device is configured to test to see if the next communication operation causes a higher-than-expected voltage dip. If yes, it is likely faulty battery. If no, it is likely faulty valve circuit. Note: This test request may be generated externally or by the internal processor.

In some embodiments, the device or a system comprising the device is configured to check if a battery voltage is measured when under minimal load or no communications or valve operations are occurring. In that case an expected voltage changes over battery lifetime will be accounted for. The expected voltage dip changes over battery lifetime.

In some embodiments, one or more failure modes can be listed by the system as Critical Risk. In situations of critical risk, the device or the system may immediately generate a report for immediate transmission. Any device within the communications range (as described elsewhere in this application) will generate an immediate call to emergency services with a description of the issue.

As referred herein, the term “external response” refers to communications with external devices as described elsewhere in this application.

In an embodiment, the device comprises a method of detection for potential failure modes and an action plan after the detection of failure. In some embodiments, the implantable device may comprise one or more of: a) A piston position sensor, B) A method of measuring battery voltage, C) A pressure sensor located in the osmotic or pharmaceutical compartments, D) A real time clock or other method of maintaining relative time regardless of processor reset or exception, E) A method of communicating with a device or devices outside the body, D) A processor with watchdog timer capability, E) A method of recharging the implant's power source and appropriate methods of monitoring the recharging, and F) Temperature sensor on components such as the processor, valve, battery, and valve driver circuitry. Various embodiments may require more or fewer failure modes depending on the chosen components and subsystems of the embodiment. In an embodiment, failure related data is transmitted or stored when a failure is detected. A report may cause an immediate data communication with external resources, or the report may be stored until the next external communication or until the device is removed from the Patient and data can be downloaded. Data transmitted or stored may comprise timestamp, requested dose size and period, actual dosing size, dosing periods, piston position, component temperatures, battery voltage, current draw, and piston position over time. This data may be collected before, during and after an action such as valve actuation, communications, and waking up or going into standby. In some embodiments, the device may respond to certain failures, such as applying pulse patterns to unstick a valve or restricting flow in a stuck-open valve scenario. The severity and risks associated with each failure mode are described in the software program associated with the device, and some failure modes generate an immediate report that triggers a call to emergency services.

Accurate numerical simulations are crucial for implant safety and must treat complex implants realistically. Phantom measurements validate the simulation methodology but are not predictive for in vivo cases. Safety assessments must be specific to the subject, implant, and scan conditions. Lower field strengths do not necessarily reduce risk, as safety depends on individual factors. The implanted devices can cause safety issues when exposed to strong magnetic fields and radio waves. Healthcare providers should carefully evaluate the risks and benefits of MRI for each patient, work closely with the device manufacturer and radiologist, and take appropriate safety measures during the scan. In some cases, alternative imaging modalities may be necessary.

### DD. Possible Uses of Device

### 1. Device Used for Long-Term Sustained Release of Insulin or Analogs Thereof

In one embodiment, the present disclosure relates to a device for controlled delivery of drugs, comprising a micro or nano beads that contains drug, wherein the device is suitable for delivery of long-term sustained release of insulin or analogs thereof, GLP-1 or analogs thereof, alone or in combination with other therapies, to treat diabetes or other metabolic conditions. The examples of GLP-1 agonists drugs that can be used via the device comprise Dulaglutide, Exenatide, Exenatide, Semaglutide, Liraglutide and Lixisenatide. In an embodiment, the device stores a semaglutide drug and the medication dose per shot ranges between 0.2 mg per week to 1 mg per week.

### 2. Device Used for Long-Term Release of Contraceptive Hormones

In one aspect of this embodiment, the device is suitable for delivery of long-term release of contraceptive hormones, combination of estrogen or progestin, or singular delivery of progesterone alone, and serves as contraceptive aid for women.

### 3. Device Used for Long-Term Release of Ocular Diseases Related Therapeutic

In one aspect of this embodiment, the device is suitable for delivery of long-term therapeutic benefits for ocular diseases, such as age-related macular degeneration, dry eye, and various others.

### 4. Device Used for Long-Term Release of Drugs to Treat Urinary Bladder Complications

In one aspect of this embodiment, the device is suitable for delivery of any small molecule or biologic therapy to treat urinary bladder complications such as incontinence, yeast infections, bladder cancer and various others.

### 5. Device Used for Delivery of Drugs for Treatment Related to Male Reproductive Organs

In one aspect of this embodiment, the device is suitable for delivery of therapeutic molecules to the male reproductive organs as a means to treat medical conditions such as erectile dysfunction, premature ejaculation, testicular cancer, and various others pertaining to male reproductive system.

### 6. Device Used for Delivery of Drugs for Treatment Related to Female Reproductive Organs

In one aspect of this embodiment the device is suitable for delivery of therapeutic molecules to female reproductive organs, uterus, and ovaries, to treat medical conditions such as endometriosis, uterine fibroids, ovarian cancer, uterine cancer, poly cystic ovarian syndrome, and various other diseases pertaining to female reproductive system.

### 7. Device Used for Delivery of Drugs for Treatment Related to Heart Conditions

In one aspect of this embodiment, the device is suitable for delivery of therapeutic molecules to heart conditions such as heart failure, myocardial ischemia, and various other heart diseases.

### 8. Device Used for Delivery of Drugs for Treatment Via Adipose Tissue

In one aspect of this embodiment, the device is suitable for delivery of therapeutic molecules into the adipose tissue to treat conditions such as metabolic syndrome, diabetes, hypercholesterolemia, hypertriglyceridemia, and various others.

### 9. Device Used for Delivery of Drugs for HIV

In some embodiments, the drug combination is a combination of HIV medicines. The combination HIV medicines contain two or more HIV medicines from one or more drug classes. The combination HIV drug can be one of a combination of Abacavir and lamivudine; abacavir, dolutegravir, and lamivudine; abacavir, lamivudine, and zidovudine; atazanavir and cobicistat; bictegravir, emtricitabine, and tenofovir alafenamide; cabotegravir and rilpivirine; darunavir and cobicistat; darunavir, cobicistat, emtricitabine, and tenofovir alafenamide; dolutegravir and lamivudine; dolutegravir and rilpivirine; doravirine, lamivudine, and tenofovir disoproxil fumarate; efavirenz, emtricitabine, and tenofovir disoproxil fumarate; efavirenz, lamivudine, and tenofovir disoproxil fumarate; efavirenz, lamivudine, and tenofovir disoproxil fumarate; elvitegravir, cobicistat, emtricitabine, and tenofovir alafenamide; elvitegravir, cobicistat, emtricitabine, and tenofovir disoproxil fumarate; emtricitabine, rilpivirine, and tenofovir alafenamide; emtricitabine, rilpivirine, and tenofovir disoproxil fumarate; emtricitabine and tenofovir alafenamide; emtricitabine and tenofovir disoproxil fumarate; lamivudine and tenofovir disoproxil fumarate; lamivudine and zidovudine; and lopinavir and ritonavir.

### 10. Device Used for Renal Disease Treatment

In an embodiment, implantable drug delivery devices can be used for the treatment of renal diseases. The implantable drug delivery devices can be used in the treatment of renal diseases such as chronic kidney disease (CKD), diabetic nephropathy, and polycystic kidney disease.

In an embodiment, the implantable drug delivery devices are designed to deliver drugs directly to the site of action, which can help to minimize side effects and increase the effectiveness of treatment. In the case of renal diseases, implantable drug delivery devices can be used to deliver drugs directly to the kidneys or other parts of the urinary tract.

For example, implantable drug delivery devices can be used to deliver drugs that help to slow the progression of CKD or treat the symptoms of diabetic nephropathy. They can also be used to deliver drugs that help to shrink cysts in the kidneys in the case of polycystic kidney disease.

### 11. Device Used for Cancer Treatment

In an embodiment, implantable drug delivery devices can be used for the treatment of cancer. These devices can be used to deliver chemotherapy drugs directly to the site of the tumor, which can help to increase the effectiveness of treatment while reducing side effects.

In an embodiment, the implantable drug delivery device can be surgically implanted under the skin and comprises a programmable drug delivery component such as a pump. The implantable drug delivery device can be programmed to deliver drugs over a period of time, which can help to maintain a constant level of the drug in the body.

In the case of cancer, implantable drug delivery devices can be used to deliver chemotherapy drugs directly to the site of the tumor. This can help to reduce the amount of drug that is needed to achieve an effective dose, which can reduce the risk of side effects associated with systemic chemotherapy. Implantable drug delivery devices can also be used to deliver drugs directly to the site of a cancer recurrence or metastasis.

The implantable drug delivery device could be designed to deliver a variety of chemotherapy drugs, including paclitaxel, doxorubicin, and gemcitabine. The device could be programmed to deliver a continuous infusion of the drug over a period of time, which can help to maintain a consistent level of the drug in the body.

The device can be reloaded with additional drug as needed, which can help to extend the duration of treatment without requiring additional surgeries. The device can also be programmed to deliver different drugs at different times, which can help to optimize treatment based on the specific characteristics of the tumor.

### INCORPORATION BY REFERENCE

All publications, patents, and patent applications cited in this Specification are hereby incorporated by reference in their entirety, including:
U.S. Publication 20120222748 titled “Droplet creation techniques”
U.S. Pat. No. 7,776,927B2 titled “Emulsions and techniques for formation”
U.S. Publication 20120141589A1 titled “Particles for drug delivery and other applications”
U.S. Publication 20130202657A1 titled “Foams or particles for applications such as drug delivery”
U.S. Pat. No. 6,858,220B2 titled “Implantable microfluidic delivery system using ultra-nanocrystalline diamond coating”
U.S. Publication 20130035574A1 titled “Microfluidic drug delivery devices with venturi effect”
U.S. Publication 20130035660A1 titled “Multidirectional microfluidic drug delivery devices with conformable balloons”
U.S. Pat. No. 7,560,036B2 titled “System and method for drug delivery and microfluidic applications using microneedles”
U.S. Publication 20080187579A1 titled “Extended-release dosage form”
U.S. Pat. No. 8,343,140B1 titled “Devices, formulations, and methods for delivery of multiple beneficial agents”
U.S. Patent Publication No. 20170119854 titled “Highly concentrated drug particles, formulations, suspensions and uses thereof”
U.S. Patent Publication 20080260840 titled “Suspension formulations of insulinotropic peptides and uses thereof”
U.S. patent Ser. No. 10/159,714B2 titled “Compositions, devices and methods of use thereof for the treatment of cancers”
U.S. Pat. No. 8,367,095B2 titled “Two-piece, internal-channel osmotic delivery system flow modulator”
U.S. Publication 2022/0370774 A1 titled “Adjustable rate drug delivery implantable device”
U.S. patent Ser. No. 11/426,567 titled “Adjustable rate drug delivery implantable device”
U.S. patent Ser. No. 10/406,336 titled “Adjustable rate drug delivery implantable device”
U.S. Pat. No. 8,535,701B2 titled “Sustained delivery of an active agent using an implantable system”
U.S. patent Ser. No. 10/231,923B2 titled “Rapid establishment and/or termination of substantial steady-state drug delivery”
U.S. Pat. No. 8,801,700B2 titled “Osmotic delivery systems and piston assemblies for use therein”
U.S. Pat. No. 5,997,527A titled “Self adjustable exit port”
U.S. Pat. No. 6,132,420A titled “Osmotic delivery system and method for enhancing start-up & performance of osmotic delivery systems”
U.S. Pat. No. 6,190,350B1 titled “Implanter device for subcutaneous implants”
U.S. Pat. No. 6,287,295B1 titled “Osmotic delivery system semipermeable body assembly”
U.S. Pat. No. 6,375,978B1 titled “Rate controlling membranes for controlled implants”
U.S. Pat. No. 6,508,808B1 titled “Valve for osmotic devices”
U.S. Pat. No. 6,524,305B1 titled “Osmotic delivery system flow modulator apparatus and method”
U.S. Pat. No. 6,544,252B1 titled “Osmotic delivery system having space efficient piston”
U.S. Pat. No. 6,872,201B2 titled “Osmotic delivery system having space efficient piston”
U.S. Pat. No. 6,939,556B2 titled “Minimally compliant, volume-efficient piston for osmotic drug delivery systems”
U.S. Pat. No. 7,014,636B2 titled “Osmotic delivery device having a two-way valve and a dynamically self-adjusting flow channel”
U.S. Pat. No. 7,655,257B2 titled “Sustained delivery of an active agent using an implantable system”
U.S. Pat. No. 7,879,028B2 titled “Osmotic delivery systems and piston assemblies for use therein”
U.S. Publication 20060127925A1 titled “Stimuli-responsive polymer conjugates and related methods,”
U.S. Publication 20160263221A1 titled “Multi-responsive targeting drug delivery systems for controlled-release pharmaceutical formulation”
U.S. Publication 20170119785A1 titled “Sol-gel polymer composites and uses thereof”
U.S. Publication 20170165201A1 titled “PH-responsive mucoadhesive polymeric encapsulated microorganisms”
U.S. Publication 20170135953A1 titled “Methods for localized drug delivery,”
U.S. Publication 20170073311 titled “Suprametallogels and uses thereof,”
U.S. Publication 20170065721A1 titled “Synthesis and use of therapeutic metal ion containing polymeric particles”
U.S. Pat. No. 8,801,801B2 titled “At least partially resorbable reticulated elastomeric matrix elements and methods of making same”
U.S. Publication 20140142556A1 titled “Implantable drug delivery devices”
U.S. Pat. No. 8,403,907 titled “Method for wirelessly monitoring implanted medical device”
U.S. Pat. No. 8,308,707 titled “Method and system for drug delivery to the eye”
U.S. Pat. No. 7,918,842 titled “Medical device with controlled reservoir opening”
U.S. Pat. No. 7,901,397 titled “Method for operating microchip reservoir device”
U.S. Pat. No. 7,892,221 titled “Method of controlled drug delivery from implantable device”
U.S. Pat. No. 7,879,019 titled “Method of opening reservoir of containment device”
U.S. Pat. No. 7,776,024 titled “Method of actuating implanted medical device”
U.S. Pat. No. 7,354,597 titled “Microscale lyophilization and drying methods for the stabilization of molecules”
U.S. Pat. No. 7,226,442 titled “Microchip reservoir devices using wireless transmission of power and data”
U.S. Pat. No. 7,070,592 titled “Medical device with array of electrode-containing reservoirs”
U.S. Pat. No. 7,070,590 titled “Microchip implants”
U.S. Pat. No. 6,976,982 titled “Flexible microchip devices for ophthalmic and other applications”
U.S. Pat. No. 6,808,522 titled “Microchip devices for delivery of molecules and methods of fabrication thereof”
U.S. Pat. No. 6,537,256 titled “Microfabricated devices for the delivery of molecules into a carrier fluid”
U.S. Pat. No. 6,491,666 titled “Microfabricated devices for the delivery of molecules into a carrier fluid”
U.S. Pat. No. 6,123,861 titled “Fabrication of microchip implants”
U.S. Pat. No. 5,797,898 titled “Microchip implants”
U.S. Pat. No. 5,514,378 titled “Biocompatible polymer membranes and methods of preparation of three dimensional membrane structures”
U.S. Publication 20170020402A1 titled “Implantable and bioresorbable sensors”
U.S. Publication 20160129111A1 titled “Methods for delivering an anti-cancer agent to a tumor”
U.S. Publication 20130184640A1 titled “Accurate flow control in drug pump devices”
U.S. patent Ser. No. 10/653,836B2 titled “System for monitoring safety in medication delivery for diabetes management”
U.S. Pat. No. 8,700,181B2 titled “Single-chamber leadless intra-cardiac medical device with dual-chamber functionality and shaped stabilization intra-cardiac extension”
U.S. patent Ser. No. 10/252,063B2 titled “Leadless intra-cardiac medical device with built-in telemetry system”
U.S. patent Ser. No. 10/449,354B2 titled “Intracardiac medical device”
U.S. patent Ser. No. 11/219,760B2 titled “Compact implantable medical device and delivery device”
U.S. Publication 20210236731A1 titled “Operating multi-modal medicine delivery systems”
U.S. Publication 20140088554A1 titled “Drug-delivery pump with intelligent control”
U.S. Pat. No. 9,203,861 titled “Methods and systems for determining hardening strategies”
U.S. Pat. No. 9,436,822 titled “Virtual browsing environment”
U.S. Pat. No. 10,956,184 titled “Malware detector”
U.S. Pat. No. 9,846,588 titled “on demand disposable virtual work system”
U.S. Pat. No. 8,082,452 titled “Protecting sensitive data associations”
U.S. Publication 20100054481 titled “Scalable distributed data structure with recoverable encryption”
U.S. Pat. No. 8,566,269 titled “Interactive analysis of attack graphs using relational queries”
U.S. Publication 20100058456 titled “IDS sensor placement using attack graphs”
U.S. patent Ser. No. 11/207,504 titled “Shuttle apparatus and associated systems and methods”
U.S. Publication 20200352522 titled “Personalization of artificial intelligence models for analysis of cardiac rhythms”
U.S. Publication 20200108260 titled “Multi-tier prediction of cardiac tachyarrythmia”
U.S. patent Ser. No. 11/219,760 titled “Compact implantable medical device and delivery device”
U.S. patent Ser. No. 11/056,267 titled “Receive coil configurations for implantable medical device”
U.S. patent Ser. No. 11/045,658 titled “Receive coil configurations for implantable medical device”
U.S. patent Ser. No. 10/821,292 titled “Multi-axis coil for implantable medical device”
U.S. patent Ser. No. 11/152,819 titled “Recharge of implanted medical devices”
U.S. patent Ser. No. 10/707,692 titled “Recharge of implanted medical devices”
U.S. patent Ser. No. 11/443,852 titled “Reduced power machine learning system for arrhythmia detection”
U.S. patent Ser. No. 11/355,244 titled “Machine learning based depolarization identification and arrhythmia localization visualization”
U.S. Publication 20210338138 titled “Visualization of arrhythmia detection by machine learning”
U.S. Publication 20210338134 titled “Arrhythmia detection with feature delineation and machine learning”
U.S. patent Ser. No. 11/071,500 titled “Identification of false asystole detection”
U.S. Publication 20200155845 titled “Vfa delivery systems and methods”
U.S. patent Ser. No. 10/856,951 titled “Device retention mechanism and method”
U.S. patent Ser. No. 11/437,649 titled “Composite separator and electrolyte for solid state batteries”
U.S. Publication 20210369394 titled “Intelligent Assistance (IA) Ecosystem”
U.S. Publication 20210106837 titled “Cardiac pacing device with mechanical mode switching”
U.S. patent Ser. No. 11/475,998 titled “Data preparation for artificial intelligence-based cardiac arrhythmia detection”
U.S. Publication 20200353271 titled “Selection of probability thresholds for generating cardiac arrhythmia notifications”
U.S. patent Ser. No. 10/737,099 titled “Antenna for implantable medical devices”
U.S. patent Ser. No. 10/881,862 titled “Estimating RV-timings from left ventricular (LV) sensing times for adaptive cardiac resynchronization therapy using DDD/VDD LV pacing without a right ventricular (RV) lead”
U.S. Publication 20220398470 titled “Adjudication algorithm bypass conditions”
U.S. Publication 20220384014 titled “Cardiac episode classification”
U.S. Publication 20220314006 titled “Assessing pacemaker dependency”
U.S. Publication 20220369937 titled “Acute health event monitoring”
PCT Publication WO2022173674 titled “Health event prediction”
PCT Publication WO2022173675 titled “Medical survey trigger and presentation”
U.S. Publication 20220257283 titled “Echogenic delivery system for leadless pacemaker”
U.S. Publication 20220257184 titled “Short-term variability sensing to anticipate tachyarrhythmias”
U.S. Publication 20220211331 titled “Detection of infection in a patient”
U.S. Publication 20220160250 titled “Detection and mitigation of inaccurate sensing by an implanted sensor of a medical system”
U.S. Publication 20220088390 titled “Conduction system pacing with adaptive timing to maintain av and interventricular synchrony”
U.S. Publication 20220062645 titled “Implantable medical device with pacing capture classification”
U.S. patent Ser. No. 10/974,036B2 titled “Implantable device for long-term delivery of drugs”
U.S. Publication 20220023626 titled “Tiered detection of treatable events”
U.S. Publication 20210358606 titled “System and method for core-device monitoring”
U.S. patent Ser. No. 11/400,298 titled “Power source longevity”
U.S. Publication 20210338168 titled “Classification of pause-triggered episodes”
U.S. Publication 20210307669 titled “Cardiac signal qt interval detection”
U.S. Publication 20210212586 titled “Rechargeable cardiac monitor device”
U.S. Publication 20210128005 titled “Triggering storage of electrocardiographs for detected premature ventricular contractions (pvcs)”
U.S. Publication 20210060348 titled “Cardiac resynchronization therapy mode switching using mechanical activity”
U.S. Publication 20200383597 titled “Premature ventricular contraction (pvc) detection”
U.S. Publication 20200352521 titled “Category-based review and reporting of episode data”
U.S. Publication 20200129773 titled “Facilitating acceleration of advertising rates for medical devices”
U.S. patent Ser. No. 10/874,850 titled “Impedance-based verification for delivery of implantable medical devices”
U.S. patent Ser. No. 10/772,142 titled “Wireless connection to peripheral device based on advertisement cadence”
U.S. Publication 20220361758 titled “Drug administration devices that communicate with external systems and/or other devices”
U.S. patent Ser. No. 11/406,832 titled “Patient-intermediated therapy management”
U.S. patent Ser. No. 11/331,497 titled “Cardiac signal T-wave detection”
U.S. Publication 20220032068 titled “Medical system for therapy adjustment”
U.S. patent Ser. No. 10/925,728 titled “Prosthetic heart valve delivery systems and methods”
U.S. patent Ser. No. 11/278,727 titled “Efficient delivery of multi-site pacing”
U.S. patent Ser. No. 10/751,542 titled “Power management for implantable medical device systems”
U.S. patent Ser. No. 11/311,734 titled “Leadless pacing device for His bundle and bundle branch pacing”
U.S. patent Ser. No. 11/511,119 titled “Impedance sensing”
U.S. Publication 20190058214 titled “Polymer solution electrolytes”
U.S. patent Ser. No. 10/987,517 titled “Detection of noise signals in cardiac signals”
U.S. patent Ser. No. 10/532,213 titled “Criteria for determination of local tissue latency near pacing electrode”
U.S. patent Ser. No. 10/272,248 titled “Electrogram-based control of cardiac resynchronization therapy”
U.S. patent Ser. No. 10/786,302 titled “Method for closure and ablation of atrial appendage”
U.S. patent Ser. No. 10/898,707 titled “Securing an implantable medical device in position while reducing perforations”
U.S. patent Ser. No. 10/646,717 titled “Efficient delivery of multi-site pacing”
U.S. patent Ser. No. 11/197,996 titled “Delivery device for delivery of implantable or insertable medical devices”
U.S. Publication 20220361954 titled “Extended Intelligence for Cardiac Implantable Electronic Device (CIED) Placement Procedures”
U.S. patent Ser. No. 10/433,927 titled “Hinged long sealed tray and method”
U.S. Pat. No. 9,267,617B2, titled, “Drive unit for a micro valve comprising a shape memory alloy, and micro valve”
U.S. patent Ser. No. 10/935,152B2 titled: Valve having an actuator made of a shape memory alloy, with a flat geometry;
U.S. Publication 20070075286A1, titled “Piezoelectric valves drive”
U.S. Pat. No. 7,322,376B2, titled “Piezoelectric valve”
U.S. Publication 20090242813A1, titled “Piezoelectric Valve”
U.S. Publication 20040003786A1, titled, “Piezoelectric valve actuation”
U.S. Pat. No. 7,607,641B1, tilted, “Microfluidic valve mechanism”
U.S. Pat. No. 7,981,386B2, titled, “Mechanically-actuated microfluidic valve”
U.S. Pat. No. 8,092,761B2, titled, “Mechanically-actuated microfluidic diaphragm valve”
U.S. patent Ser. No. 10/072,640B2, titled “Driving device of micropump and microvalve, and microfludic device using same”
U.S. patent Ser. No. 10/935,152B2. Titled, “Valve having an actuator made of a shape memory alloy, with a flat geometry”
U.S. patent Ser. No. 11/136,968B2, titled, “Actuator assembly”
U.S. Publication 20060147329A1, titled, “Active valve and active valving for pump”
U.S. Publication 20130068325A1, titled, “Valve, layer structure comprising a first and a second valve, micropump and method of producing a valve”
U.S. Pat. No. 9,897,233B2, titled, “Micro valve device and valve body assembly”
U.S. Pat. No. 8,556,373B2, tilted “Multichannel-printhead or dosing head”
U.S. Pat. No. 9,487,008B2, titled, “Multichannel multinozzle printhead”
U.S. Publication 20060243934A1, titled, “Methods and devices for modulating fluid flow in a micro-fluidic channel”
U.S. Pat. No. 7,118,086B1, titled, “Pinch valve with inlet and outlet ports”
U.S. patent Ser. No. 10/145,476B2, titled, “Pinch valve systems and methods”
U.S. Pat. No. 7,775,501B2, titled, “Pinch Valve”
U.S. patent Ser. No. 11/491,274 titled “Liquid medicament reservoir empty detection sensor and occlusion sensor for medicament delivery device”
U.S. Publication 20040127844 titled “Flow condition sensor assembly for patient infusion device”.
U.S. Publication 20220258935 titled “System and methods for the production of personalized drug products” PCT Publication 2000066201 titled “Implantable drug delivery system”
U.S. Pat. No. 8,536,122 titled “Acylated GLP-1 compounds”
U.S. Pat. No. 9,266,940 titled “Double-acylated GLP-1 derivatives”
U.S. Pat. No. 8,129,343 titled “Acylated GLP-1 compounds”
U.S. patent Ser. No. 10/335,462 titled “Use of long-acting GLP-1 peptides”
U.S. Pat. No. 9,474,780 titled “GIP and GLP-1 co-agonist compounds”
U.S. Publication 20220387402 titled “Combinations comprising benzodioxol as glp-lr agonists for use in the treatment of nash/nafld and related diseases”
U.S. Pat. No. 8,486,352 titled “Micro-valve structure including polymer actuator and lab-on-a-chip module”
U.S. Publication 6059815 titled “Microfabricated therapeutic actuators and release mechanisms therefor”
U.S. patent Ser. No. 10/427,159 titled “Microfluidic device”
U.S. Pat. No. 6,773,566 titled “Electrostatic actuators for microfluidics and methods for using same”
U.S. Publication 20190015841A1 titled “Method and apparatus for sorting particles”
U.S. patent Ser. No. 11/229,910 titled “Microfluidic devices and systems for cell culture and/or assay”
U.S. Pat. No. 9,265,733 titled “Drug depots having different release profiles for reducing, preventing or treating pain and inflammation”
U.S. patent Ser. No. 11/439,709 titled “Phospholipid ether analogs as cancer-targeting drug vehicles”
U.S. Publication 5616123 titled “Delivery of solid drug compositions”
U.S. Pat. No. 9,402,807 titled “Therapeutic agent preparations for delivery into a lumen of the intestinal tract using a swallowable drug delivery device” PCT Publication 2020060023 titled “User-customized drug infusion system”
U.S. Publication 20060063719 titled “Methods for treating diabetes”
U.S. Pat. No. 8,414,497 titled “Detection of hypovolemia using implantable medical device”
U.S. Publication 20080161666 titled “Analyte devices and methods”
U.S. patent Ser. No. 11/690,577 titled “Systems and methods for dynamically and intelligently monitoring a host's glycemic condition after an alert is triggered”
U.S. Pat. No. 7,766,830 titled “System for monitoring physiological characteristics”
U.S. Publication 2012197148 titled “Heart rate variability sensor”
U.S. Pat. No. 8,615,282 titled “Analyte sensor”
U.S. Publication 20120059232 titled “Implantable optical glucose sensing”
U.S. Publication 20170281771 titled “Methods of using adipose tissue-derived cells in the treatment of cardiovascular conditions”
U.S. patent Ser. No. 10/603,650 titled “Multiplexed surface enhanced Raman sensors for early disease detection and in-situ bacterial monitoring”
U.S. Publication 20190231240 titled “System for Maintaining or Improving Wellness”
U.S. Publication 20120316461 titled “In situ pressure monitor and associated methods”
U.S. Pat. No. 8,774,884 titled “Systems, devices, and methods including a dark-field reflected-illumination apparatus”
U.S. Publication 20190254607 titled “Low profile medical device with bonded base for electrical components”
U.S. Publication 20220079474 titled “Implantable biosensor and methods of use thereof”
U.S. Publication 2023029054 titled “Non-invasive systems and methods for in-situ photobiomodulation”
U.S. 63/331,409 titled “An integrated wireless sensor for physiological monitoring”
U.S. Publication 2018085057 titled “Health monitor system, sensor, and method”
U.S. Publication 2020205895 titled “Non-invasive skin contact sensor”
U.S. patent Ser. No. 10/231,667 titled “Non-invasive dehydration monitoring”
U.S. patent Ser. No. 10/849,545 titled “Acute kidney injury detection system and methods”
U.S. Publication 2022002803 titled “Acute kidney injury-specific biomarker, acute kidney injury diagnosis method, acute kidney injury test kit, animal treatment method and acute kidney injury medication”.
U.S. Pat. No. 9,022,055 titled “Assembling a check valve having a seal”
U.S. Publication 4706705 titled “Check valve”
U.S. Publication 20220225940 titled “Low profile medical device with integrated flexible circuit and methods of making the same”
U.S. Publication 2023224521 titled “Tube-in-tube merger”

1.-39. (canceled)

40. A system comprising an implantable device having from 3 mm to 6 mm diameter tubular form, wherein the implantable device is inserted subcutaneously and non-surgically by an injector device in a human body via an incision; the implantable device comprising a casing comprising a pump, a piston, a drug chamber comprising a drug, and an opening for release of the drug from the implantable device into a body of a subject; wherein the implantable device is configured to be located in a subcutaneous region within the body of the subject during delivery of the drug into the body of the subject; wherein the implantable device is configured to deliver multiple doses of the drug within the body of the subject with a dose-to-dose variation of ±25% or less by volume; and wherein the implantable device further comprises power supply and electronics; and wherein the implantable device is configured to notify the subject in case of failure of the implantable device.

41. The system of claim 40, wherein the implantable device comprises:
A. a casin that is substantially tubular and has at least a first end and a second end opposite to the first end,
B. a semi-permeable membrane plug at or near the first end,
C. a first chamber comprising an osmotic agent, wherein one wall of the first chamber comprises the semi-permeable plug,
D. a second chamber comprising a drug,
E. a third chamber comprising a flow switch and at least one drug delivery orifice,
F. a piston separating the first chamber and the second chamber,
G. a fourth chamber comprising an electronic control unit and a source of energy,
wherein an osmotic pressure is configured to be built on ingress of a liquid into the first chamber through the semi-permeable membrane plug that displaces the piston towards the second chamber; and
wherein the flow switch and the osmotic pressure together regulate a release of the drug from the at least one drug orifice, such that the flow switch in its ON state allow the drug to be released from the at least one drug orifice and in its OFF state stops release of the drug from at least one drug orifice, wherein the implantable device further comprises an electro-mechanical actuator to control ON/OFF state of the flow switch based on a displacement of the piston.

42. The system of claim 41, wherein the electro-mechanical actuator comprises one of an electroactive polymer and a piezo element.

43. The system of claim 41, wherein the fourth chamber further comprises a piston position determination module, wherein the piston position determination module is configured to determine a real-time positioning of the piston and wherein the piston determination module is based on pressure measurement, conductance measurement, resistance measurement, pressure measurement, reflection measurement, capacitance measurement, impedance measurement, radical measurement, image-based measurement, laser measurement, SONAR based measurement, ultrasound measurement, time of flight measurement or combinations thereof.

44. The system of claim 43, wherein the piston position determination module interacts with one or more electro-mechanical actuator to control an ingress flow of the liquid inside the implantable device and an egress flow of the drug outside the implantable device.

45. The system of claim 41, wherein the implantable device further comprises one or more biosensors wherein one or more biosensors is configured to either detect a biomarker present in a human or an animal body, a concentration of the drug release from the implantable device and/or a bio-chemical parameter of the human or an animal body.

46. The system of claim 41, wherein a pressure sensor and/or a conductivity sensor present in the implantable device is configured to measure a displacement of the piston.

47. The system of claim 46, wherein an electric circuit connects a sensor and the one or more electro-mechanical actuator to electronic control unit in the fourth chamber to control the flow of the drug from the implantable device.

48. The system of claim 41, wherein an egress rate of the drug release is substantially same as an ingress rate of the liquid and wherein the ingress rate of the liquid in the implantable is in a range of about 0.5 μl/min to 2 μl/min.

49. The system of claim 40, further comprising:
a body temperature stable drug formulation that ensures that the drug does not degrade within the body of the subject at a body temperature of the subject for at least 6 months;
an implantable biosensor that provides real-time monitoring of a drug level and/or a health-related biomarker; and
an artificial intelligence system that integrates and analyzes data to optimize drug delivery in real-time;
wherein the system is configured to be an AI-based implantable drug delivery system.

50. A system comprising a software implemented module and an implantable device comprising:
A. a casin that is substantially tubular and has at least a first end and a second end opposite to the first end,
B. a semi-permeable membrane plug at or near the first end,
C. a first chamber comprising an osmotic agent, wherein one wall of the first chamber comprises the semi-permeable plug,
D. a second chamber comprising a drug,
E. a third chamber comprising a flow switch and at least one drug delivery orifice,
F. a piston separating the first chamber and the second chamber,
G. a fourth chamber comprising an electronic control unit and a source of energy, and
H. one or more sensors to detect amount of the drug released from the implantable device;
wherein an osmotic pressure is configured to be built on ingress of a liquid into the first chamber through the semi-permeable membrane plug that displaces the piston towards the second chamber, which further pushes the drug present in the second chamber to be release from the at least one drug orifice, and
wherein one or more sensors interact with the software implemented module to control the flow of the drug released from the implantable device.

51. The system of claim 50, wherein the system further comprises one or more biosensors configured to detect a biomarker and/or a biochemical parameter of a human or an animal body.

52. The system of claim 50, wherein the software implemented module is configured to use an artificial intelligence algorithm to analyze data provided by the one or more sensors and/or biosensors and regulate a drug release pattern from the implantable device.

53. A method comprising: receiving data from one or more sensors present on an implantable device, transmitting the data to one or more software implemented module, analyzing the data and providing an output, wherein the output regulates ingress of a liquid inside the implantable device through a semi-permeable plug present at or near first end, and egress of a drug stored in the implantable device through one or more drug orifices into a body of a human and an animal.

54. The method of claim 53, wherein the one or more software implemented module comprises at least one of a data collection unit, a data processing unit, a prediction unit, and/or a dosage recommendation unit.

55. The method of claim 53, wherein the one or more sensors collects data on a biochemical parameter of the body of the human and the animal in which the implantable device is implanted.

56. The method of claim 55, wherein the data collection unit is configured to collect the data from the one or more sensors and send the data further to the data processing unit.

57. The method of claim 54, wherein the data processing unit is configured to analyze a physiological condition of the body of the human and the animal in which the implantable device is implanted.

58. The method of claim 53, wherein the prediction unit is configured to utilize a machine learning algorithm to provide a drug dosing pattern according to the physiological condition of the body of the human and the animal.

59. The method of claim 53, wherein the method is configured to provide a personalized drug dosing pattern according to a physiological condition of the body of the human and the animal in which the implantable device is implanted.
